(19) United States
(12) Patent Application Publication
(10) Pub. No.: US 2026/0263495 A1
(43) Pub. Date: Sep. 10, 2026

(54) METHODS AND BIOAVAILABLE HIGHLY PERMEABLE COMPOUNDS FOR DISEASES TREATMENT

(71) Applicant: Kirill Didenko, Cancun (MX)

(72) Inventor: Kirill Didenko, Cancun (MX)

(21) Appl. No.: 19/165,608

(22) PCT Filed: Mar. 18, 2024
(86) PCT No.: PCT/IB2024/052611

§ 371 Date: Sep. 16, 2025

(51) Int. Cl. A61K 31/7048; A61K 9/10; A61K 31/365; A61K 31/4184; A61K 31/426; A61K 47/02; A61K 47/38; A61K 47/46; A61K 47/58; A61P 35/00

(52) U.S. Cl. CPC A61K 31/7048; A61K 9/10; A61K 31/365; A61K 31/4184; A61K 31/426; A61K 47/02; A61K 47/38; A61K 47/46; A61K 47/58; A61P 35/00

(57) ABSTRACT

Methods and compositions are provided for treating diseases by administering bioavailable, highly permeable API-based non-covalent complexes, solid dispersions or conjugates. The compounds, compositions and methods are particularly useful for treating diseases.

### FIELD OF THE INVENTION

The invention relates generally to methods and compounds for treating diseases, particularly methods and compounds based on APIs for treating diseases.

### BACKGROUND OF THE INVENTION

Infectious diseases pose a significant threat to public health worldwide, causing substantial damage both in terms of human suffering and economic burden. These diseases, ranging from common colds to more severe conditions like HIV/AIDS and Ebola, have the potential to wreak havoc on communities and healthcare systems. The damage inflicted by infectious diseases extends beyond the direct effects on individual health, impacting productivity, socioeconomic stability, and healthcare infrastructure.

One of the primary ways infectious diseases cause harm is through their ability to spread rapidly within populations, leading to outbreaks and pandemics. Such events can overwhelm healthcare systems, strain resources, and result in significant morbidity and mortality. Additionally, infectious diseases can have long-term consequences, including chronic health conditions and disabilities, further exacerbating the burden on individuals and society as a whole.

Moreover, the economic toll of infectious diseases is profound, encompassing direct healthcare costs, lost productivity, and investments in prevention and control measures. In regions with limited resources and inadequate healthcare infrastructure, the impact of infectious diseases can be particularly devastating, perpetuating cycles of poverty and inequality. Addressing the damage caused by infectious diseases requires a comprehensive approach that encompasses prevention, surveillance, early detection, and access to effective treatments and vaccines.

Metabolic diseases, such as diabetes, obesity, and metabolic syndrome, inflict significant damage on both individual health and society at large. These conditions disrupt the body's normal metabolic processes, leading to complications that affect various organs and systems. For instance, diabetes can result in cardiovascular diseases, kidney failure, blindness, and nerve damage, significantly reducing quality of life and life expectancy for those affected.

Furthermore, the economic burden of metabolic diseases is staggering, encompassing direct healthcare costs, loss of productivity, and expenses related to managing complications. The prevalence of these conditions is on the rise globally, driven by factors such as sedentary lifestyles, unhealthy diets, and aging populations. Addressing the damage caused by metabolic diseases requires a multifaceted approach, including public health interventions to promote healthier lifestyles, improved access to healthcare services, and research into new treatments and preventive strategies.

Neurodegenerative diseases, such as Alzheimer's, Parkinson's, and Huntington's, inflict profound damage on individuals and society, both in terms of human suffering and economic burden. These diseases gradually impair cognitive function, motor skills, and overall neurological health, leading to progressive disability and dependency. Beyond the personal toll on affected individuals and their families, neurodegenerative diseases strain healthcare systems, require extensive caregiving resources, and diminish productivity. Moreover, as populations age worldwide, the prevalence of these conditions is expected to increase, highlighting the urgent need for innovative treatments, better support services, and enhanced public awareness and education initiatives.

Cardiovascular diseases exact a heavy toll on both individuals and society, manifesting in a range of conditions such as coronary artery disease, stroke, and hypertension. These diseases not only contribute to significant morbidity and mortality but also impose substantial economic burdens through healthcare costs and lost productivity. The damage caused by cardiovascular diseases extends beyond the physical realm, impacting emotional well-being, social dynamics, and overall quality of life for affected individuals and their families. Effective prevention strategies, early detection, and comprehensive management approaches are crucial in mitigating the devastating effects of cardiovascular diseases and reducing their societal impact.

Diseases inflict damage not only on individuals but also on society as a whole, posing significant challenges to healthcare systems, economies, and overall well-being. Whether they are infectious, metabolic, neurodegenerative, or cardiovascular in nature, diseases disrupt normal physiological functions, impairing physical health, mental health, or both. Beyond the immediate health consequences, they often lead to financial strain, decreased productivity, and social disruption. Highly effective medications play a pivotal role in the treatment of diseases by providing targeted and potent therapeutic interventions that can significantly improve patient outcomes and quality of life. These medications are often the result of rigorous scientific research and development processes, leveraging advancements in pharmacology, biotechnology, and medical innovation. By targeting specific disease mechanisms, reducing symptoms, and preventing complications, highly effective drugs not only alleviate suffering but also contribute to the overall reduction of disease burden on individuals and society. Furthermore, they enable healthcare providers to offer personalized treatment approaches, tailored to individual patient needs, leading to more precise and efficient healthcare delivery. Overall, the availability and utilization of high-efficacy medications represent a cornerstone in modern medicine's ability to combat various diseases and enhance patient care.

### SUMMARY OF THE INVENTION

### Technical Problem

The transition from in vitro studies to clinical trials often reveals a significant efficacy gap in drug delivery systems. While in vitro studies provide valuable insights into the mechanisms and potential efficacy of a drug, translating these results into clinical settings presents considerable challenges. Factors such as physiological barriers, systemic clearance, and biodistribution dynamics can drastically alter the performance of a drug in vivo compared to the controlled conditions of a laboratory environment.

Addressing the efficacy gap between in vitro studies and clinical trials necessitates the development of sophisticated drug delivery systems. These systems aim to bridge the disparity by ensuring that the drug reaches the targeted sites in the body at levels comparable to those observed in vitro. Various approaches, including nanoparticles, liposomes, and microparticles, have been explored to enhance drug delivery efficiency and efficacy.

Furthermore, the design of drug delivery systems must consider factors such as stability, biocompatibility, and controlled release kinetics to optimize therapeutic outcomes. By employing innovative technologies and leveraging our understanding of biological systems, researchers strive to narrow the efficacy gap and facilitate the successful translation of promising drug candidates from the laboratory bench to the patient bedside.

Therefore, there is an efficacy gap between in vitro studies and clinical trials requires a drug delivery system with which to achieve the in vitro levels.

### Solution to Problem

The present invention proposes to destroy the crystallinity of the phase, the phase itself and the phase boundaries of API and to form chemical bonds (including hydrogen bonding, van der Waals bonding, donor-acceptor bonding, π-π bonding, Ion-dipole bonding and ion-induced dipole bonding, ionic bonding, hydrophobic bonding and others) between API molecules and the carrier and thereby change the properties of API by forming new compounds from it. In this invention, it is found that during solvation, the carrier molecules (as part of non-covalent or inclusion or supramolecular complex with API) can in some cases modify the biological/cell membrane by adhesion or by insertion into the lipid membrane or using other transport mechanism and promote the transport (permeability) of API or change in hydrophilicity and hydrophobicity, due to which changes in the paracellular transport. That changes in permeability can occur due to:
1) as a consequence of blocking/reducing the activity of CYP3A4 and/or P-glycoproteins, blocking/reducing the activity of other transmembrane carrier proteins, transporters, enzymes, etc., by membrane interacting with carrier substance molecules;
2) due to blocking/reduction of CYP3A4 and/or P-glycoprotein activity, blocking/reduction of activity of other transmembrane protein transporters, transporters, enzymes, etc., by carrier substance molecules interacting with the membrane due to adhesion of carrier substance molecules to the cellular layer. Such adhesion can lead to interaction with transporters domains located on the outer part of the cell membrane and block the action of the transmembrane transporter protein;
3) due to a change in the structure of the cell membrane after the incorporation of carrier substance molecules into this membrane, rather than due to adhesion;
4) due to the direct effect of carrier substance molecules on the intestinal epithelium cell membrane: it may consist in alteration of lipid bilayer properties, density of intercellular contacts or inhibition of trans-membrane proteins of multidrug resistance;
5) due to changes in hydrophilicity and hydrophobicity due to which changes in the paracellular transport of the non-covalent complex may occur
6) other changes in the structure and properties of the membrane, through other mechanisms that contribute to an increase in its permeability;
7) due to the use of various transmembrane transport mechanisms.

A number of methods to destroy the crystallinity of the phase are generally referred to as “Molecular/Solid Dispersion Methods” and subdivided into a number of sub-methods, all of which involve molecular mixing and disordered phase processes of the components at the molecular level and create a homogeneous/highly dispersed phase with many chemical interactions between the API and the carrier.

Molecular/Solid dispersions of the present inventions includes inclusion complexes and supramolecular complexes (that can present in solid phase) that can present in form of solid solution in which molecules of one compound (API, carrier) occupy places in the crystal lattice of another compound (API, carrier) or in which one compound (carrier) spatially encloses another (API). Thus in solid dispersions of the invention not only mixing at the molecular level but also complexation can take place.

Molecular/Solid dispersion methods such as the solvent evaporation, cryogenic methods, melting methods and hot-stage extrusion methods are used to create compounds with the described properties. In contrast to melting methods in the method of milling with high-energy input/stress, the required processes take place locally in places of excess energy created by mechanical forces (e.g. at the moment of collision of balls there are shear, compression and impact). The processes of milling with high energy input/stress are cumulative processes:
a) destruction of the structure of the initial phases;
b) solid-state formation of chemical bonds between invermectin and the carrier;
c) solid-state phase transformations including solid-phase fusion with formation of fusion phases (including nonequilibrium);
d) accumulation of structural defects;
e) dispersion, disarrangement and homogenization;
and the result is a gradual synthesis of the necessary compounds.

It should be understood that milling in high-energy conditions is not done for grinding, but for energy impact, which provides the formation of new melting phases, and the size of the particles can increase, not decrease. During high-energy milling the powder particles are repeatedly flattened, cold welded, fractured and rewelded. Whenever two steel balls collide, some powder is trapped between them. Typically, around 1000 particles with an aggregate weight of about 0.2 mg are trapped during each collision. The force of the impact plastically deforms the powder particles, leading to work hardening and fracture. The new surfaces thus created enable the particles to weld together; this leads to an increase in particle size. Since in the early stages of milling, the particles are soft (if using either ductile-ductile or ductile-brittle material combination), their tendency to weld together and form large particles is high. A broad range of particle sizes develops, with some as large as three times larger than the starting particles.

The present invention discloses that the molecular/solid dispersion treatment of API acquires new molecular properties that are not identical to the pharmaceutical composition. When combined into a pharmaceutical composition, each of the substances retains its molecular properties in the same form as they were before being combined into the pharmaceutical composition and does not change its molecular composition. At the same time, obtaining a solid dispersion produces a qualitatively different molecular substance that permanently changes its basic molecular properties due to the formation of chemical bonds between the molecules of the carrier and API. Because API and the carrier together form a new single structure, the properties of the API solid dispersion method are different from and not equivalent to a mixture of API with any other substance. The use of solid dispersion method involves difficulties in selecting the carrier, solvent and processing conditions, it is necessary to achieve processing parameters in which the substances do not disintegrate or degrade, it is necessary to control the toxicity of the resulting compounds, since in some cases using some carriers, the toxicity of the resulting compounds may increase. In general, using the solid dispersion method is a complex process with many factors.

When treated by the solid dispersion method, API acquires new molecular properties that are not identical to the pharmaceutical composition, namely modified pharmacokinetic parameters, different system bioavailability, in vivo permeability, reduced by 3-5 times the toxicity, the treatment time of the infection is reduced to 1-2 days at a single dose, the new compounds have different spectra from the simple mixture. Compounds obtained by solid dispersion methods can have any size (like alloys), can be dissolved in solvents, can be made into various compositions and formulations, can be combined with other preparations, i.e. have all the properties of a separate substance per se.

Provided are methods and compounds for the disease treatment. Provided are methods (co-treating methods/solid dispersion techniques) for modifying the properties of a compound (API) by creating a molecular/solid dispersion or inclusion complex (compound of Formula II, using as precursors API, host substance and, if necessary, other compounds) with properties of increased bioavailability and permeability and using compounds obtained by this methods (or aqueous solutions or suspensions thereof) to manufacture a pharmaceutical dosage form to treat cancer in a human in need thereof (by administering a therapeutically effective amount) or to treat a cancer
wherein the host substance is selected from:
a) polymers and oligomers, predominantly organic polymers and oligomers, even more predominantly polysaccharides and oligosaccharides, hemicelluloses, storage polysaccharides, sulfated polysaccharides and oligosaccharides, pectins, gums, mucilages, or mixtures thereof; b) substances that may contain in significant amounts polysaccharides and oligosaccharides, hemicelluloses, storage polysaccharides, sulfated polysaccharides and oligosaccharides, pectins, gums, mucilages, which may be (but are not limited to) plants or algae or animal or fungi, parts of plants or algae or animal or fungi, processed plants or algae or animal or fungi, processed parts of plants or algae or animal or fungi containing in significant amounts the substances specified in paragraph a), or mixtures thereof; c) syntetic polymers, predominantly water-soluble polymers or mixtures thereof; d) polyols or mixtures thereof; e) saccharides or mixtures thereof; f) surfactants or mixtures thereof; g) acids or mixtures thereof; h) oxides and salts based on these oxides or mixtures thereof; i) copolymers of the polymers listed in paragraph a) and c); j) methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in a)-i), and their esters, salts, and any other chemical derivatives, and mixtures thereof; k) any combination of substances specified in paragraph a)-j);
wherein co-treating method is selected from:
a) grinding/milling with high energy stress method; b) grinding/milling with high energy stress and solvent method; c) media milling method; d) kneading method; e) hot-melt method/melting method/fusion method; f) hot-melt extrusion/hot-stage extrusion method; g) meltrex method; h) melt agglomeration method; i) high-pressure homogenization method; j) solvent evaporation method; k) spin-coated films method; l) spray-drying method; m) supercritical fluid (SCF) process method; n) cryogenic techniques method; o) lyophilization/freeze-drying technique method; p) spray freezing onto cryogenic fluids method; q) spray freezing into cryogenic liquids (SFL) method; r) spray freezing into vapor over liquid (SFV/L) method; s) ultra-rapid freezing method; t) precipitation/co-precipitation method; u) microwave irradiation method; v) energy input method; w) heat/shear energy input method; x) combined method.

### Advantageous Effects of Invention

When treated by the solid dispersion method, API acquires new molecular properties that are not identical to the pharmaceutical composition, namely modified pharmacokinetic parameters, increased bioavailability, permeability, reduced by 3-5 times the toxicity.

### BRIEF DESCRIPTION OF DRAWINGS

FIG. 1 Polymer repeat units.

FIG. 2 Polymer repeat units.

FIG. 3 Polymer repeat units.

FIG. 4 Polymer repeat units.

FIG. 5 Polymer repeat units.

FIG. 6 Monosaccharides.

FIG. 7 Monosaccharides.

FIG. 8 Radicals.

FIG. 9 NCC examples.

FIG. 10 NCC examples.

FIG. 11 NCC examples.

FIG. 12-15 APIs used for NCC, SD, conjugates.

### DETAILED DESCRIPTION OF THE INVENTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the claims.

### I. DEFINITIONS OF THE PRESENT INVENTION

The term “Diseases of the blood and blood-forming organs and certain disorders involving the immune mechanism”, as used herein, means any disease of the blood and blood-forming organs and certain disorders involving the immune mechanism listed in The International Statistical Classification of Diseases and Related Health Problems (including ICD-9, ICD-10 (Chapter III Diseases of the blood and blood-forming organs and certain disorders involving the immune mechanism (D50-D89)), ICD-10-CM, ICD-11 (03 Diseases of the blood or blood-forming organs and 04 Diseases of the immune system), ICD-11-CM, and others);
The term “Endocrine, nutritional and metabolic diseases”, as used herein, means any endocrine, nutritional and metabolic disease listed in The International Statistical Classification of Diseases and Related Health Problems (including ICD-9, ICD-10 (Chapter IV Endocrine, nutritional and metabolic diseases (E00-E90)), ICD-10-CM, ICD-11 (05 Endocrine, nutritional or metabolic diseases), ICD-11-CM, and others);
The term “Mental and behavioural disorders”, as used herein, means any mental and behavioural disorders listed in The International Statistical Classification of Diseases and Related Health Problems (including ICD-9, ICD-10 (Chapter V Mental and behavioural disorders (F00-F99)), ICD-10-CM, ICD-11 (06 Mental, behavioural or neurodevelopmental disorders and 07 Sleep-wake disorders), ICD-11-CM, and others) or any disorder or condition listed in The Diagnostic and Statistical Manual of Mental Disorders, Fifth or IV Edition (DSM-5, DSM-5-TR, DSM-IV-TR);
The term “Diseases of the nervous system”, as used herein, means any disease of the nervous system listed in The International Statistical Classification of Diseases and Related Health Problems (including ICD-9, ICD-10 (Chapter VI Diseases of the nervous system (G00-G99)), ICD-10-CM, ICD-11 (08 Diseases of the nervous system), ICD-11-CM, and others);
The term “Diseases of the eye and adnexa”, as used herein, means any disease of the eye and adnexa listed in The International Statistical Classification of Diseases and Related Health Problems (including ICD-9, ICD-10 (Chapter VII Diseases of the eye and adnexa (H00-H59)), ICD-10-CM, ICD-11 (09 Diseases of the visual system), ICD-11-CM, and others);
The term “Diseases of the ear and mastoid process”, as used herein, means any diseases of the ear and mastoid process listed in The International Statistical Classification of Diseases and Related Health Problems (including ICD-9, ICD-10 (Chapter VIII Diseases of the ear and mastoid process (H60-H95)), ICD-10-CM, ICD-11, ICD-11-CM, and others);
The term “Diseases of the circulatory system”, as used herein, means any disease of the circulatory system listed in The International Statistical Classification of Diseases and Related Health Problems (including ICD-9, ICD-10 (Chapter IX Diseases of the circulatory system (I00-I99)), ICD-10-CM, ICD-11 (11 Diseases of the circulatory system), ICD-li-CM, and others);
The term “Diseases of the respiratory system”, as used herein, means any disease of the respiratory system listed in The International Statistical Classification of Diseases and Related Health Problems (including ICD-9, ICD-10 (Chapter X Diseases of the respiratory system (J00-J99)), ICD-10-CM, ICD-11, ICD-11-CM, and others);
The term “Diseases of the digestive system”, as used herein, means any disease of the digestive system listed in The International Statistical Classification of Diseases and Related Health Problems (including ICD-9, ICD-10 (Chapter XI Diseases of the digestive system (K00-K93)), ICD-10-CM, ICD-11, ICD-11-CM, and others);
The term “Diseases of the skin and subcutaneous tissue”, as used herein, means any disease of the skin and subcutaneous tissue listed in The International Statistical Classification of Diseases and Related Health Problems (including ICD-9, ICD-10 (Chapter XII Diseases of the skin and subcutaneous tissue (L00-L99)), ICD-10-CM, ICD-11, ICD-11-CM, and others);
The term “Diseases of the musculoskeletal system and connective tissue”, as used herein, means any disease of the musculoskeletal system and connective tissue listed in The International Statistical Classification of Diseases and Related Health Problems (including ICD-9, ICD-10 (Chapter XIII Diseases of the musculoskeletal system and connective tissue (M00-M99)), ICD-10-CM, ICD-11, ICD-11-CM, and others);
The term “Diseases of the genitourinary system”, as used herein, means any disease of the genitourinary system listed in The International Statistical Classification of Diseases and Related Health Problems (including ICD-9, ICD-10 (Chapter XIV Diseases of the genitourinary system (N00-N99)), ICD-10-CM, ICD-11, ICD-11-CM, and others);

The term “parasitic diseases”, as used herein, means any protozoa, unicellular, apicomplexa, algae parasites, macroparasites (worms, helminths, flukes, etc), ectoparasites and arthropods parasitic diseases listed in The International Statistical Classification of Diseases and Related Health Problems (including ICD-9, ICD-10 (Chapter I Certain infectious and parasitic diseases (A00-B99)), ICD-10-CM, ICD-11 (01 Certain infectious or parasitic diseases), ICD-11-CM, and others) and includes case of Disease vector control. Non-limiting examples: Onchocerciasis (due to Onchocerca volvulus), Lymphatic filariasis (due to Wuchereriabancrofti), Strongyloidiasis (due to Strongyloidesstercoralis), Scabies (due to Sarcoptesscabiei), Crusted scabies (Norwegian Scabies), Pediculosis (due to Pediculuscapitis, Pediculuscorporis, Pediculus pubis), Demodicosis (due to Demodexfolliculorum and Demodexbrevis), Demodicosis (due to Demodexfolliculorum and Demodexbrevis), Filariasis (due to Mansonellaozzardi), Filariasis (due to Mansonellastreptocerca), Gnathostomiasis (due to Gnathostomaspinigerum), Cutaneous larva migrans (due to Ancylostomabraziliense), Trichuriasis (due to Trichuristrichiura), Ascariasis (due to Ascarislumbricoides, Enterobiasis (due to Enterobiusvermicularis), Myiasis, Trichinosis, Schistosomiasis, Bedbugs, Rosacea, Buruli ulcer, and for Disease vector control (non limiting example): Malaria, Dengue, Leishmaniasis, African trypanosomiasis (sleeping sickness), American trypanosomiasis (Chagas disease).

The term “fungal diseases”, as used herein, means any fungal medical conditions or diseases or disorders or syndromes or infections caused by fungi, including listed in The International Statistical Classification of Diseases and Related Health Problems (including ICD-9, ICD-10 (Chapter I Certain infectious and parasitic diseases (A00-B99)), ICD-10-CM, ICD-11 (01 Certain infectious or parasitic diseases), ICD-11-CM, and others). Non-limiting examples: Aspergillosis; Basidiobolomycosis; Blastomycosis; Candidosis; Chromoblastomycosis; Coccidioidomycosis; Conidiobolomycosis; Cryptococcosis; Dermatophytosis; Eumycetoma; Histoplasmosis; Lobomycosis; Mucormycosis; Non-dermatophyte superficial dermatomycoses; Paracoccidioidomycosis; Phaeohyphomycosis; Pneumocystosis; Scedosporiosis; Sporotrichosis; Talaromycosis; Emmonsiosis;

And those fungal disease caused by fungi belonging to the order Mucorales or genus Acremonium; Aspergillus; Aspergillus; Candida; Cladophialophora; Coccidioides; Cryptococcus; Cryptococcus; Curvularia; Cylindrocarpon; Exophiala; Falciformispora; Fusarium; Glenospora; Histoplasma; Leptosphaeria; Lomentospora; Madurella; Microsporum; Nakaseomyces; Neotestudina; Paracoccidioides; Phaeoacremonium; Phialophora; Pichia; Pneumocystis; Pyrenochaeta; Scedosporium; Talaromyces; Trichophyton; Zopfia. And those fungal disease caused by fungi belonging to the species (non-limiting example): Cryptococcus neoformans; Candida auris; Aspergillus fumigatus; Candida albicans; Nakaseomyces glabrata (formerly Candida glabrata); Histoplasma capsulatum; Histoplasma capsulatum var. capsulatum; Histoplasma capsulatum var. duboisii; Histoplasma capsulatum var. farciminosum; Histoplasma duboisii; Histoplasma muris; Acremonium falciform; Acremonium kiliense; Acremonium recifei; Aspergillus flavus; Aspergillus nidulans; Cladophialophora bantiana; Cladophialophora mycetomatis; Curvularia geniculata; Curvularia lunata; Cylindrocarpon cyanescens; Exophiala jeanselmei; Falciformispora senegalensi; Fusarium moniliforme; Fusarium solani; Glenospora clapieri; Leptosphaeria senegalensis; Leptosphaeria tompkinsii; Madurella grisea; Madurella mycetomatis; Microsporum audouinii; Microsporum canis; Neotestudina rosatii; Phaeoacremonium parasiticum; Phialophora cyanescens; Phialophora verrucosa; Scedosporium (ex. Pseudoalleschia) boydii; Pyrenochaeta mackinonii; Pyrenochaeta romeroi; Trichophyton rubrum; Zopfia rosatii; Candida tropicalis; Candida parapsilosis; Scedosporium americanum; Scedosporium apiospermum; Scedosporium aurantiacum; Scedosporium boydii; Scedosporium cereisporum; Scedosporium deficiens; Scedosporium dehoogii; Scedosporium desertorum; Scedosporium magalhaesii; Scedosporium minutisporum; Scedosporium prolificans; Scedosporium rarisporum; Scedosporium sanyaense; Coccidioides immitis; Coccidioides posadasii; Lomentospora prolificans; Pichia kudriavzeveii (formerly Candida krusei); Cryptococcus gattii; Talaromyces marneffei; Pneumocystis jirovecii; Paracoccidioides americana; Paracoccidioides brasiliensis; Paracoccidioides cerebriformis; Paracoccidioides ceti; Paracoccidioides lutzii; Paracoccidioides restrepoana; Paracoccidioides venezuelensis.

The term “bacterial diseases”, as used herein, means any bacterial medical conditions or diseases or disorders or syndromes or infections caused by bacteria, including those caused by bacteria belonging to the genus: Abiotrophia; Acetobacter; Acholeplasma; Achromobacter; Acidaminococcus; Acidomonas; Acidovorax; Acinetobacter; Actinobacillus; Actinobaculum; Actinomadura; Actinomyces; Actinotignum; Advenella; Aerococcus; Aeromonas; Afipia; Aggregatibacter; Agrobacterium; Alcaligenes; Alistipes; Alkanindiges; Alloiococcus; Alloprevotella; Alloscardovia; Amycolatopsis; Anaerobiospirillum; Anaerococcus; Anaeroglobus; Anaerostipes; Anaplasma; Aquimonas; Arcanobacterium; Arcobacter; Arthrobacter; Asaia; Atlantibacter; Atopobium; Atopobium; Aureimonas; Auritidibacter; Bacillus; Bacteroides; Balneatrix; Bartonella; Bergeyella; Bifidobacterium; Bilophila; Bisgaardia; Bordetella; Borrelia; Brachybacterium; Brachyspira; Branchiibius; Brevibacillus; Brevibacterium; Brevundimonas; Brucella; Budvicia; Bulleidia; Burkholderia; Buttiauxella; Butyricimonas; Campylobacter; Canibacter; Capnocytophaga; Cardiobacterium; Catabacter; Catonella; Cedecea; Cellulomonas; Cellulosimicrobium; Centipeda; Chitinophaga; Chlamydia; Chlamydophila; Chromobacterium; Chryseobacterium; Citrobacter; Clostridioides; Clostridium; Collinsella; Comamonas; Coprobacillus; Corynebacterium; Coxiella; Cronobacter; Cruoricaptor; Cryptobacterium; Cupriavidus; Curtobacterium; Cutibacterium; Delftia; Dermabacter; Dermacoccus; Dermatophilus; Desmospora; Desulfomicrobium; Desulfovibrio; Dialister; Dichelobacter; Dietzia; Diplorickettsia; Dokdonella; Dolosigranulum; Dyella; Dysgonomonas; Edwardsiella; Effusibacillus; Eggerthella; Eggerthia; Ehrlichia; Eikenella; Eisenbergiella; Elizabethkingia; Empedobacter; Enterobacter; Enterococcus; Erwinia; Erysipelatoclostridium; Erysipelothrix; Escherichia; Eubacterium; Ewingella; Exiguobacterium; Facklamia; Faecalicatena contorta; Fastidiosipila; Fenollaria; Fibrobacter; Filifactor; Finegoldia; Flavobacterium; Fluoribacter; Francisella; Frederiksenia; Fretibacterium; Fusobacterium; Gallibacterium; Gardnerella; Gemella; Globicatella; Gordonia; Gordonibacter; Granulibacter; Granulicatella; Haematobacter; Haematomicrobium; Haematospirillum; Haemophilus; Hafnia; Halomonas; Hathewaya; Hazenella; Helcobacillus; Helcococcus; Helicobacter; Herbaspirillum; Hungatella; Ignatzschineria; Ignavigranum; Inquilinus; Janibacter; Jonquetella; Kerstersia; Kingella; Klebsiella; Kluyvera; Kocuria; Kosakonia; Kytococcus; Lachnoanaerobaculum; Lactobacillus; Lactococcus; Laribacter; Lawsonella; Leclercia; Legionella; Leifsonia; Lelliottia; Leminorella; Leptospira; Leptotrichia; Leuconostoc; Listeria; Luteibacter; Luteococcus; Lysinibacillus; Malacobacter; Mannheimia; Massilia; Megamonas; Megasphaera; Metamycoplasma; Methylobacterium; Microbacterium; Micrococcus; Microvirgula; Mobiluncus; Moellerella; Mogibacterium; Moraxella; Morganella; Moryella; Murdochiella; Mycobacterium; Mycoplasma; Mycoplasmoides; Mycoplasmopsis; Myroides; Necropsobacter; Negativicoccus; Neisseria; Neisseria; Neoehrlichia; Neorickettsia; Nocardia; Nocardiopsis; Oblitimonas; Ochrobactrum; Odoribacter; Oerskovia; Oligella; Oribacterium; Orientia; Orientia; Oscillibacter; Paenalcaligenes; Paenibacillus; Pandoraea; Pantoea; Parabacteroides; Paraclostridium; Paracoccus; Paraeggerthella; Parapseudoflavitalea; Parvimonas; Pasteurella; Peptococcus; Peptoniphilus; Peptostreptococcus; Phocaeicola; Photobacterium; Photorhabdus; Plesiomonas; Pluralibacter; Pontibacter; Porphyromonas; Prescottia; Prevotella; Propionibacterium; Proteus; Providencia; Pseudoclavibacter; Pseudomonas; Pseudonocardia; Pseudopropionibacterium; Pseudoramibacter; Pseudoxanthomonas; Psychrobacter; Pyramidobacter; Rahnella; Ralstonia; Raoultella; Rhizobium; Rhodococcus; Rickettsia; Robinsoniella; Rodentibacter; Roseomonas; Rothia; Ruminococcus; Saccharomonospora; Saccharopolyspora; Saezia; Salmonella; Scandinavium; Scardovia; Sciscionella; Sebaldella; Sedimentibacter; Segniliparus; Selenomonas; Serratia; Shewanella; Shigella; Shuttleworthia; Simkania; Slackia; Sneathia; Sodalis; Solobacterium; Sphingobacterium; Sphingomonas; Spiroplasma; Staphylococcus; Stenotrophomonas; Stomatobaculum; Streptobacillus; Streptococcus; Streptomyces; Sutterella; Suttonella; Tannerella; Tatlockia; Tatumella; Tepidimonas; Terrisporobacter; Tissierella; Treponema; Tropheryma; Trueperella; Tsukamurella; Turicella; Turicibacter; Ureaplasma; Vagococcus; Varibaculum; Veillonella; Vibrio; Waddlia; Wautersiella; Weeksella; Weissella; Williamsia; Wohlfahrtiimonas; Wolinella; Xenophilus; Yersinia; Yokenella;
a comprehensive list of bacterial pathogens species can be found in “A comprehensive list of bacterial pathogens infecting humans”, Bartlett et al., Microbiology 2022; 168:001269, DOI 10.1099/MIC.0.001269, DOI: 10.5281/ZENODO.7337535, but not excluding other bacteria;
and are selected from those listed in The International Statistical Classification of Diseases and Related Health Problems (including ICD-9, ICD-10 (Chapter I Certain infectious and parasitic diseases (A00-B99)), ICD-10-CM, ICD-11 (01 Certain infectious or parasitic diseases), ICD-11-CM, and others);
and/or wherein a person's medical conditions/diseases/disorders/syndromes are selected from those listed in The Diagnostic and Statistical Manual of Mental Disorders, including DSM-IV, DSM-5, DSM-5-TR, and others;
and/or wherein a person's medical conditions/diseases/disorders/syndromes are selected from those listed in the Medical Subject Headings and beginning with MeSH codes C01-C25;
and/or wherein a person's medical conditions/diseases/disorders/syndromes are selected from conditions that are published in the Genetic and Rare Diseases (GARD) Information Center;

The term “viral diseases”, as used herein, means any viral medical conditions or diseases or disorders or syndromes or infections caused by viruses, including those caused by RNA viruses, DNA viruses and reverse transcribing viruses; including those caused by viruses belonging to the realm Duplodnaviria, Monodnaviria, Adnaviria, Ribozyviria, Riboviria, and Varidnaviria; including those caused by dsDNA viruses, ssDNA viruses, dsRNA viruses, (+)ssRNA viruses, (−)ssRNA viruses, ssRNA-RT viruses, dsDNA-RT viruses; including viral infections or viral diseases caused by viruses included in the families Coronaviridae, Flaviviridae, Herpesviridae, Orthomyxoviridae, Paramyxoviridae, Polyomaviridae, Retroviridae, Togaviridae, but not excluding other viruses families; including viral infections or viral diseases caused by Human SARS coronavirus, SARS coronavirus 2, Dengue virus, West Nile virus, Yellow fever virus, Zika virus, Bovine alphaherpesvirus 1, Influenza A virus, Hendra virus, BK polyomavirus, Human immunodeficiency virus type 1, Chikungunya virus, Semliki forest virus, Sindbis virus, Venezuelan equine encephalitis virus, Human adenovirus, Junin arenavirus, Lassa virus, Lymphocytic choriomeningitis virus, Machupo virus, Pichinde virus, Porcine reproductive and respiratory syndrome virus (PRRSV)/Betaarterivirus suid 1, Human astrovirus, Hantaan virus, Crimean-Congo hemorrhagic fever virus, Dugbe virus, Bunyamwera virus, Bunyavirus La Crosse, Bunyavirus snowshoe hare, Punta toro phlebovirus, Rift valley fever virus, Sandfly fever Naples phlebovirus (Toscana virus), Sandfly fever sicilian virus, Uukuniemi virus, Norwalk virus, Southampton virus, Sapporo virus, porcine circovirus 2, Human coronavirus, MERS coronavirus, Human torovirus, Ebola virus, Lake Victoria marburg virus, Japanese encephalitis virus, Langat virus, Louping ill virus, St. louis encephalitis virus, Tick-borne powassan virus, TBE virus, Hepatitis C virus, GB virus C/Hepatitis G virus, Hepatitis B virus, Hepatitis E virus, Human cytomegalovirus, Cercopithecine herpesvirus, Epstein-Barr virus, Human herpesvirus 8, Human herpesvirus 6, Human herpesvirus 7, Human herpesvirus 1, Human herpesvirus 2, Varicella-zoster virus, Bovine alphaherpesvirus 1, Equine herpesvirus type 1 (EHV-1), Pseudorabies virus, Rubella virus, Influenza B virus, Influenza C virus, Influenza A virus, Dhori virus, Human papillomavirus (16,18), Human papillomavirus 2, Human papillomavirus 1, Nipah virus, Measles virus, Human parainfluenza, Mammalian orthorubulavirus 5 (Simian virus 5), Mumps virus, Newcastle, Adeno-associated virus, Human parvovirus B19, Encephalomyocarditis virus, Cosavirus A, Coxsackievirus, Echovirus, Human enterovirus (68, 70), Poliovirus, Hepatitis A virus, Aichi virus, Rosavirus A, Rhinovirus, Salivirus A, Human respiratory syncytial virus, JC polyomavirus, KI Polyomavirus, Merkel cell polyomavirus, WU polyomavirus, BK polyomavirus, Molluscum contagiosum virus, Cowpox virus, Horsepox virus, Vaccinia virus, Variola virus, Yaba monkey tumor virus, Yaba-like disease virus, Smallpox, Orf virus, Rotavirus A, Rotavirus B, Orbivirus, Coltivirus, Rotavirus C, Banna virus, Human T-lymphotropic virus, Human immunodeficiency virus, Eastern chimpanzee simian foamy virus, Simian foamy virus, Human immunodeficiency virus type 1/2, Australian bat lyssavirus, Duvenhage virus, Lagos bat virus, Mokola virus, Rabies virus, Chandipura virus, Isfahan virus, Vesicular stomatitis virus, European bat lyssavirus, Barmah forest virus, Eastern equine encephalitis virus, Mayaro virus, O'nyong-nyong virus, Ross river virus, Sagiyama virus, Western equine encephalitis virus, Rubella virus, Hepatitis delta virus, Hepatitis E virus, Torque teno virus, Human rhinovirus, Murray valley encephalitis virus, Oropouche virus, Monkeypox virus, New York virus, Puumala virus, Seoul virus, but not excluding other viruses;
and are selected from those listed in The International Statistical Classification of Diseases and Related Health Problems (including ICD-9, ICD-10 (Chapter I Certain infectious and parasitic diseases (A00-B99)), ICD-10-CM, ICD-11 (01 Certain infectious or parasitic diseases), ICD-11-CM, and others);
and/or wherein a person's medical conditions/diseases/disorders/syndromes are selected from those listed in The Diagnostic and Statistical Manual of Mental Disorders, including DSM-IV, DSM-5, DSM-5-TR, and others;
and/or wherein a person's medical conditions/diseases/disorders/syndromes are selected from those listed in the Medical Subject Headings and beginning with MeSH codes C01-C25;
and/or wherein a person's medical conditions/diseases/disorders/syndromes are selected from conditions that are published in the Genetic and Rare Diseases (GARD) Information Center;

The term “cancer”, as used herein, means any cancer (tumor, neoplasm), Bone and muscle sarcoma cancers; Chondrosarcoma; Ewing's sarcoma; Malignant fibrous histiocytoma of bone/osteosarcoma; Osteosarcoma; Rhabdomyosarcoma; Leiomyosarcoma; Myxosarcoma; Fibrocartilaginous mesenchymoma of bone; Brain and nervous system cancers; Astrocytoma; Brainstem glioma; Pilocytic astrocytoma; Ependymoma; Primitive neuroectodermal tumor; Cerebellar astrocytoma; Cerebral astrocytoma; Glioblastoma; Glioma; Medulloblastoma; Neuroblastoma; Oligodendroglioma; Pineal astrocytoma; Pituitary adenoma; Visual pathway and hypothalamic glioma; Breast cancers; Breast cancer; Inflammatory breast cancer; Invasive lobular carcinoma; Tubular carcinoma; Invasive cribriform carcinoma of the breast (also termed invasive cribriform carcinoma); Medullary carcinoma; Male breast cancer; Phyllodes tumor; Mammary secretory carcinoma; Papillary carcinomas of the breast; Endocrine system cancers; Adrenocortical carcinoma; Islet cell carcinoma (endocrine pancreas); Multiple endocrine neoplasia syndrome; Pancreatic Cancer; Parathyroid cancer; Pheochromocytoma; Thyroid cancer; Merkel cell carcinoma; Eye cancers; Uveal melanoma; Retinoblastoma; Optic nerve glioma; Gastrointestinal cancers; Anal cancer; Appendix cancer; Cholangiocarcinoma; Carcinoid tumor, gastrointestinal; Colon cancer; Duodenal cancer; Extrahepatic bile duct cancer; Gallbladder cancer; Gastric (stomach) cancer; Gastrointestinal carcinoid tumor; Gastrointestinal stromal tumor (GIST); Hepatocellular cancer; Pancreatic cancer, islet cell; Rectal cancer; Small intestine cancer; Genitourinary and gynecologic cancers; Bladder cancer; Cervical cancer; Endometrial cancer; Extragonadal germ cell tumor; Ovarian cancer; Ovarian epithelial cancer (surface epithelial-stromal tumor); Ovarian germ cell tumor; Penile cancer; Kidney cancer; Renal cell carcinoma; Renal pelvis and ureter, transitional cell cancer; Prostate cancer; Testicular cancer; Gestational trophoblastic tumor; Ureter and renal pelvis; transitional cell cancer(urothelial carcinoma); Urethral cancer; Uterine sarcoma; Vaginal cancer; Vulvar cancer; Wilms tumor (nephroblastoma); Head and neck cancers; Esophageal cancer; Head and neck cancer; Nasopharyngeal carcinoma; Oral cancer; Oropharyngeal cancer; Paranasal sinus and nasal cavity cancer; Pharyngeal cancer; Salivary gland cancer; Hypopharyngeal cancer; Hematopoietic cancers; Acute biphenotypic leukemia; Acute eosinophilic leukemia; Acute lymphoblastic leukemia; Acute myeloid leukemia; Acute myeloid dendritic cell leukemia; AIDS-related lymphoma; Anaplastic large cell lymphoma; Angioimmunoblastic T-cell lymphoma; B-cell prolymphocytic leukemia; Burkitt's lymphoma; Chronic lymphocytic leukemia; Chronic myelogenous leukemia; Cutaneous T-cell lymphoma; Diffuse large B-cell lymphoma; Follicular lymphoma; Hairy cell leukemia; Hepatosplenic T-cell lymphoma; Hodgkin's lymphoma; Intravascular large B-cell lymphoma; Large granular lymphocytic leukemia; Lymphoplasmacytic lymphoma; Lymphomatoid granulomatosis; Mantle cell lymphoma; Marginal zone B-cell lymphoma; Mast cell leukemia; Mediastinal large B cell lymphoma; Multiple myeloma/plasma cell neoplasm; Myelodysplastic syndromes; Mucosa-associated lymphoid tissue lymphoma; Mycosis fungoides; Nodal marginal zone B cell lymphoma; Non-Hodgkin lymphoma; Precursor B lymphoblastic leukemia; Primary central nervous system lymphoma; Primary cutaneous follicular lymphoma; Primary cutaneous immunocytoma; Primary effusion lymphoma; Plasmablastic lymphoma; Sézary syndrome; Splenic marginal zone lymphoma; T-cell prolymphocytic leukemia; Skin cancers; Basal cell carcinoma; Squamous cell carcinoma; Squamous cell skin cancer; Skin adnexal tumors (e.g. sebaceous carcinoma); Melanoma; Merkel cell carcinoma; Keratoacanthoma; Sarcomas of primary cutaneous origin (e.g. dermatofibrosarcoma protuberans); Lymphomas of primary cutaneous origin (e.g. mycosis fungoides); Thoracic and respiratory cancers; Adenocarcinoma of the lung; Basaloid squamous cell lung carcinoma; Bronchial adenomas/carcinoids; Small cell lung cancer; Mesothelioma; Non-small cell lung cancer; Non-small cell lung carcinoma; Pleuropulmonary blastoma; Laryngeal cancer; Thymoma and thymic carcinoma; Squamous-cell carcinoma of the lung; HIV/AIDS related cancers; AIDS-related cancers; Kaposi sarcoma; Epithelioid hemangioendothelioma (EHE); Desmoplastic small round cell tumor; Liposarcoma;
and any malignant tumors such as Carcinoma, Sarcoma, Lymphoma, Leukemia, Germ cell tumor, Blastoma (e.g. neuroblastoma, retinoblastoma, nephroblastoma, hepatoblastoma, medulloblastoma, etc.), Melanoma, Endothelioma, Glioma of tissues, organs, parts of organs, parts of the body, skin, muscles, bones, connective tissue, internal organs, organs of vision, speech, hearing, smell, taste:
a. skin;
b. head; eye; ear; nose; mouth; tongue; teeth; lower jaw; face; cheek;
c. neck; throat; Adam's apple; shoulders;
d. hand; elbow; wrist; hand; fingers; thumb;
e. spine; breast; mammary gland; rib cage;
f. abdomen; navel; genitals (penis/scrotum or clitoris/vagina); perineum; anus;
g. leg; pelvis; thigh; buttock; knee; shin; calf; foot—toes;
h. hair, nails;
i. pituitary; duodenum; stomach; gallbladder; intestines; lungs; uterus; bladder; adrenals; parathyroid; liver; pancreas; kidneys; prostate; esophagus; spleen; heart; thymus; worm gland; thyroid; testes; ovaries;
j. brain;
k. anatomical parts included in “Terminologia Anatomica The Second Edition”, Parts I-V, Chapters 1-16, which is the international standard for human anatomical terminology;
and any person's cancer medical conditions or diseases or disorders or syndromes are selected from those listed in The International Statistical Classification of Diseases and Related Health Problems (including ICD-9, ICD-10 (Chapter II Neoplasms (C00-D48)), ICD-10-CM, ICD-11 (02 Neoplasms), ICD-11-CM, and others);
and any person's cancer medical conditions or diseases or disorders or syndromes are selected from those listed in The Diagnostic and Statistical Manual of Mental Disorders, including DSM-IV, DSM-5, DSM-5-TR, and others;
and any person's cancer medical conditions or diseases or disorders or syndromes are selected from those listed in the Medical Subject Headings 2023 and its MeSH TreeNumber code starts with MeSH codes C01-C25;
and any person's cancer medical conditions or diseases or disorders or syndromes are selected from conditions that are published in the Genetic and Rare Diseases (GARD) Information Center.

The term “treatment” as used herein, unless otherwise specified, means the abolition, relief, inhibition of the progression or prevention of the disorder or condition to which the term applies, or one or more symptoms of such disorder or condition. The term “treatment”, as used herein, refers to the action of treatment as “treating” is defined directly above.

The term “therapeutically effective amount”, as used herein, is the amount of a compound of Formula I-V present in a composition described herein that is necessary to provide the desired level of the drug in the secretions and tissues of the respiratory tract and lungs or, alternatively, in the bloodstream of the subject to be treated to produce the expected physiological response or desired biological effect when such composition is administered in the selected route of administration. The exact amount depends on a variety of factors, such as the specific compound of Formula I-V, the specific activity of the composition, the delivery device used, the physical characteristics of the composition, its intended use, and patient considerations such as severity of the disease state, patient cooperation, etc., and can be readily determined by a person skilled in the art based on the information provided herein.

The term “active pharmaceutical ingredient” (API, guest, guest drug, guest drug compound, guest compound, guest extract, guest biologically active extract) is pharmaceutically active and bioactive compound; if the selected guest drug is in the form of a free base—the pharmaceutical salts of that base and the pharmaceutical ester or ether are also included; if the guest drug is in the form of a pharmaceutical salt—the free base of that salt, the pharmaceutical ester or ether, and other pharmaceutical salts of that free base are also included; also include racemate, enantiomer, diastereomer, tautomer, polymorph, pseudopolymorph, hydrate, isotopically labeled forms, prodrugs thereof or mixtures thereof.

The term “additional active pharmaceutical ingredient” (AAPI, guest, guest drug, guest drug compound, guest compound, guest extract, guest biologically active extract) is a pharmaceutically active and biologically active compound that is administered with an API to produce an additional therapeutic effect.

The term “additional active natural pharmaceutical ingredient” (AANPI, guest, guest drug, guest drug compound, guest compound, guest extract, guest biologically active extract) is pharmaceutically active and bioactive compound from natural sources, primary or secondary metabolite that is administered with an API to produce an additional therapeutic effect. AANPI may be in the form of a pure substance isolated from a natural source, in the form of an extract, a dried extract, or in the form of a processed natural source, such as a powder and any other form in which it is contained.

The term “derivative” of active pharmaceutical ingredient (of API, AAPI, AANPI, guest, guest drug, guest drug compound, guest compound) means derivatives such as racemate, enantiomer, diastereomer, tautomer, polymorph, pseudopolymorph, hydrate, isotopically labeled forms, prodrugs, pharmaceutical salts, pharmaceutical ester or ether. If the API in the form of a free base, the derivatives are the pharmaceutical salts of that base and the pharmaceutical ester or ether. If the API is in the form of a pharmaceutical salt—the free base of that salt, the pharmaceutical ester or ether, and other pharmaceutical salts of that free base are also derivatives. Derivatives can also be other obvious derivatives—chemical and physical derivatives (see below).

The term “prodrug” is defined in the pharmaceutical field as a biologically inactive derivative of a drug that, when introduced into the human body, is transformed into a biologically active parent drug according to some chemical or enzymatic pathway.

The term “solvent”, as used herein, means organic and inorganic solvents which may be (but are not limited to): water, hydrocarbons and their halogen derivatives, alcohols, esters and ethers, ketones, nitro compounds: 1,1,1-trichloroethane, 1,1,2-trichloroethene, 1,1-dichloroethene, 1,2-dichloroethane, 1,2-dichloroethene, 1,2-dimethoxyethane, 1,2-propanedioi, 1,4-dioxane, 1-butanol, 1-pentanol, 1-propanol, 2-butanol, 2-butanone, 2-ethoxyethanol, 2-methoxyethanol, 2-methyl-1-propanol, 2-methylpyridine, 2-methyltetrahydrofuran, 2-nitropropane, 2-propanol, 3-methyl-1-butanol, 4-methyl-2-pentanone, acetic acid, acetic anhydride, acetone, acetonitrile, acetophenone, ammonia, aniline, anisole, benzene, benzonitrile, benzylalcohol, bromoethane, bromoform, bromooctane, butanol, butanone, butylacetate, butylbenzene, carbon disulfide, carbontetrachloride, carbontet, cellosolve acetate, cetic acid, chlorobenzene, chloroform, chloroform, chlorohexane, cumene, cyclohexanes cyclohexane, cyclohexanol, cyclohexanone, cyclopentane, cyclopentanes, decalin, decane, decanol, dibromoethane, dibutylether, dichlorobenzene, difluorobenzene, dichloroethane, dichloromethane, diethyleneglycol, diethylether, diglyme (diethylene glycoldimethyl ether), diisopropylether, dimethoxyethane, dimethyl acetate, dimethyl sulfoxide, dimethylacetamide, dimethylformamide, dimethylpyridine, dimethylsulfoxide, dioxane, dioxanes (1.4 dioxane), dodecane, esters, etbe, ethanediol, ethanol, ether(s), ethoxybenzene, ethyl acetate, ethyl acetoacetate, ethyl alcohol, ethylacetate, ethylbenzene, ethylene glycol ethyl ether, ethylene glycol methyl ether, ethylene glycol monobutyl ether, ethyleneglycol, ethylether, ethylformate, fluorobenzene, fluoroctane, formamide, formic acid, freon, furfuraldehyde, glacial acetic acid, glycerin, glycol ethers, halobenzenes, heptane, heptanes, heptanol, hexadecane, hexadecyliodide, hexamethylphosphoramide, hexamethylphosphorous triamide (hmpt), hexane, hexanes, hexanol, i-amyl alcohol, iodobenzene, isobutanol, isobutyl acetate, isooctane, isopropanol, isopropyl acetate, isopropylbenzene, isopropyltoluene, meresol, mesitylene, methanoic acid, methanol, methoxyethanol, methyl acetate, methyl ethyl ketone, methyl t-butyl ether, methyl tert butyl ether, methylbutyl ketone, methylcyclohexane, methylenechloride, methylethyl ketone, methylformamide, methylisobutyl ketone, miscellaneous solvents, monochlorobenzene, morpholine, m-xylene, n,n-dimethylacetamide, n,n-dimethylformamide, n-amyl alcohol, n-butyl acetate, nitrobenzene, nitroethane, nitromethane, n-methyl-2-pyrrolidinone(nmp), n-methylpyrrolidone, n-octanol, nonane, nonanol, octane, octanol, odichlorobenzene, onitrotoluene, o-xylene, pentadecane, pentane, pentanes, pentanol, perchloroethylene, perfluorobenzene, petroleum ether (ligroine), phenol, phenylether, propanol, propoxypropane, propyl acetate, propylene glycol methyl ether, p-xylene, pyridine, secbutanol, secbutylbenzene, sulfolane, t-butanol, t-butyl alcohol, tbutylbenzene, tert-butylmethylether, tetrachloroethane, tetrachloroethylene, tetrachloromethane, tetrahydrofuran, tetrahydrothiophenedioxide, tetralin, toluene, tributylphosphate, trichloroethane, trifluoromethylbenzene, trichloroethylene, triethylamine, trimethylbenzene, trimethylpentane, undecane, xylene, xylenes, 2,2,4-trimethyl pentane, and mixtures thereof. The term “physiologically accepted solvent” typically refers to a solvent that is considered safe for use in biological and physiological systems. In various scientific and medical applications, solvents are used for dissolving or diluting substances, and it's important that these solvents do not cause harm to living organisms. Here are some common examples:
a. Water: Water is the most fundamental and widely used solvent in biological systems. It is essential for various physiological processes and is the basis for many biological fluids.
b. Saline Solution: Saline solution is a mixture of salt (sodium chloride) dissolved in water. It is commonly used as an intravenous fluid to replenish electrolytes and maintain hydration.
c. Ethanol and other alchohols: Ethanol, or ethyl alcohol, is a type of alcohol that is sometimes used in medical and laboratory settings. It is commonly used as a disinfectant and can be used in certain pharmaceutical preparations.
d. Glycerol: Glycerol, also known as glycerin, is a colorless and odorless liquid that is often used as a solvent in pharmaceuticals, cosmetics, and food. It is considered safe for use in various physiological applications.
e. Propylene Glycol: Propylene glycol is a synthetic liquid that is commonly used in the pharmaceutical and food industries. It is used as a solvent in oral, injectable, and topical drug formulations.
f. Dimethyl Sulfoxide (DMSO): DMSO is a solvent that is known for its ability to penetrate biological tissues. It is used in medical and laboratory settings for certain applications, such as cryopreservation of cells.

The term “physically modified” or “physical derivative” means use of physical methods for modification of involves use of mechanical forces, sound, ultrasound, dry heat, microwave technology, UV and gamma radiations. Generally natural carriers on modification using heating method (thermally modified derivative) will leads to change in its physical characteristics like viscosity, density, swelling index, water holding capacity, flow properties etc. Due to changes in these characteristics the improved results were obtained in case of modified natural carriers.

The term “chemically modified” or “chemical derivative” means use of chemical methods for modification. Non-limiting examples:
A. Methoxylated, ethoxylated, esterificated, carboxylated, alkoxylated, acetylated, hydroxylated, hydrated, hydrolyzed, decarboxylated, amide, oxidized, sulfated, aminoacid derivatives, fermented derivatives, acid modified derivatives, and their esters, salts, and the like;
B. Acetyl; Anhydro, D-alanyl; N-acetyl-D-alanyl; N-acetimidoyl; N—(N-methyl-acetimidoyl); N—(N,N-dimethyl-acetimidoyl); formyl; glycolyl; N-acetyl-glutaminyl; N-methyl-5-glutamyl; glycyl; glyceryl; 2,3-di-O-methyl-glyceryl; 4-hydroxybutyryl; 3,4-dihydroxybutyryl; (R)-3-hydroxybutyryl; (S)-3-hydroxybutyryl; lactyl; methyl; amino; N-acetyl; phosphate; pyruvyl; 1-carboxyethylidene; sulfate; tauryl derivatives; Alkyl derivatives: Methyl, ethyl, . . . dodecyl derivatives; Aliphatic carboxylic acids derivatives: Formyl, acetyl, glycoloyl, propionyl; Butyryl, valeryl; Hexanoyl, heptanoyl, octanoyl; Nonanoyl, decanoyl, undecanoyl; Lauroyl, myristoyl, palmitoyl; Stearoyl, eleostearoyl, linoleoyl, arachidonoyl; Glycerol derivatives and its oxidation products: Glycerol, glyceraldehyde, glycerone, glyceric acid; Glycosyl derivatives: Glucose, galactose, fucose, and the like; Gluconic acid, glucuronic acid; Glucosaminef, N-acetylglucosamine; Neuraminic, sialic, muramic acids; N-Acetylneuraminic acid, N-glycoloylneuraminic acid; Other derivatives: Ceramide, choline, ethanolamine; Inositol, serine, other aminoacids; Phosphatidyl, sphingosine, sphingoid, Phosphoric derivatives, and the like;
C. any other chemically modified derivatives which are described in the scientific and patent literature.

Others non-limiting examples:
a) Carboxymethylation/carbomoylethylation in which free —OH groups replacement were done which leads to improved aqueous drug solubility. Carboxymethylation of natural carriers increases their hydrophilicity and makes them more soluble in aqueous systems.
b) Modification, such as the removal of a terminal sialic acid to expose glucose, fucose, mannose, galactose and the like;
c) Starting with an polysaccharide, acid catalyzed, base catalyzed or enzyme catalyzed hydrolysis may be used to produce lower molecular weight polysaccharides;
d) Embodiments of the invention also include derivatives of polymer, such as fragments, dimers, trimers, and polymers of polymer;
e) These derivatives provide a substrate for attaching therapeutic agents through attachment to the amino, carboxyl, sulfhydryl, phosphoryl or other functional groups of the derivative. The carboxyl groups that naturally occur on the terminal glycoside of polymer can be used to attach other molecules through the use of carbodiimides or other agents. Amine derivatives can also be used to attach therapeutic agents by a variety of reactions. Alternatively, dextran or poly-L-lysine can be attached to the polymer carrier to provide an increased number of sites of attachment for the therapeutic agent.

The term “avermectin”, as used herein, means avermectins and their derivatives such as: Avermectins; Avermectin A1a; Avermectin A1b; Avermectin A2a; Avermectin A2b; Avermectin B1a; Avermectin B1b; Avermectin B2a; Avermectin B2b; Ivermectins; Ivermectin B1a; Ivermectin B1b; Ivermectin A1a; Ivermectin A1b; Milbemycins; Milbemycin A3; Milbemycin A4; Milbemycin D; Milbemycin B2; Milbemycin B3; Milbemycin G; Nemadectins; Nemadectin; Meilingmycins; Meilingmycin A; Milbemectins; Milbemectin; Milbemycin oxime; Moxidectins; Moxidectin; Doramectins; Doramectin; Selamectins; Selamectin; Eprinomectins; Eprinomectin B1a; Eprinomectin B1b; Emamectins; Emamectin B1a; Emamectin B1b; Tenvermectins; Tenvermectin B; Tenvermectin A and the like; or derivatives thereof such as racemate, enantiomer, diastereomer, tautomer, polymorph, pseudopolymorph, hydride or solvate thereof; or a pharmaceutically acceptable salt or ester or ether thereof; or prodrug thereof; or methabolite thereof; or aglycone thereof; or derivatives which may be obtained by gene replacement processes in genetically engineered strains, or/and by fermentation, or/and by chemical modifications and the like, which are described in the scientific and patent literature; or mixtures thereof.

The term “disease” is mean any disease a medical conditions or diseases or disorders or syndromes or infections of a human or mammal or animal or bird has at least one of the following characteristics:
A. it is a medical condition in which functions are impaired or that is caused by impaired function at the organism level, including such functions as:
   a. sleep
   b. sexual reproduction
   c. thinking, speech, reading, writing, behavior
   d. observation, hearing, smell, sensory abilities
   e. exchange with the environment, including impaired functions:
      e.1 nutrition
      e.2 respiration
      e.3 excretion
   f. locomotion, operation of limbs and body parts
   g. others
B. it is a medical condition in which processes are disturbed or caused by disturbances in such systems and supra-systems of the body as:
   a. Musculoskeletal system
   b. Circulatory system, Cardiovascular system, Lymphatic system
   c. Nervous system
   d. Integumentary system
   e. Haematopoietic and immune systems
   f. Respiratory system
   g. Digestive system
   h. Urinary system
   i. Reproductive system
   j. Endocrine system
C. it is a medical condition in which processes are disrupted or caused by disruption of processes at the tissue/cell system level including:
   a. cell reproduction, division, differentiation, atypia, signaling, such as:
      a.1 cancer, malignant tumors such as Carcinoma, Sarcoma, Lymphoma, leukemia, Germ cell tumor, Blastoma (e.g. neuroblastoma, retinoblastoma, nephroblastoma, hepatoblastoma, medulloblastoma, etc.), Melanoma, endothelioma of tissues, organs, parts of organs, parts of the body specified in Section G;
   b. metabolism of cells and tissues
      i. cellular uptake of nutrients
      ii. sensitivity to the action of hormones, transmitters, neurotransmitters, etc., such as insulin, cortisol, dopamine, and others;
D. it is a genetic diseases, chromosomal disorder or genetic syndrome;
E. it is a medical condition that is caused or accompanied by morphological changes such as: tumor, metastases, cysts, necrosis, inflammation, accumulation of mineral salts, metabolites and etc.
F. it is a medical condition that is caused by or accompanied by:
   a. lesion or involvement or presence of infectious agents, which may be:
      i. viruses
      ii. bacteria;
      iii. fungi
      iv. parasites;
      v. ectoparasitoses;
      vi. protozoa;
      vii. prions;
   b. by lesion or involvement or presence of toxic agents;
   c. by lesion or involvement or presence of allergic agents, including Systemic allergic diseases;
   d. excessive/insufficient energy and other influences, such as:
      i. kinetic exposures and kinetic injuries;
      ii. thermal injuries;
      iii. radiation injuries;
      iv. barotraumas;
      v. other injuries;
   e. external factors:
      i. war, disasters;
      ii. violence;
      iii. stresses;
      iv. overwork;
      v. mistreatment and upbringing;
      vi. other factors;
G. it is a medical condition that affect body parts, organs, and tissues, such as parts of the body, skin, muscles, bones, connective tissue, internal organs, organs of vision, speech, hearing, smell, taste:
   a. skin;
   b. head—eye—ear—nose—mouth—tongue—teeth—lower jaw—face—cheek;
   C. neck—throat—Adam's apple—shoulders;
   d. hand—elbow—wrist—hand—fingers—thumb;
   e. spine—breast—mammary gland—rib cage;
   f. abdomen—navel—genitals (penis/scrotum or clitoris/vagina)—perineum—anus;
   g. leg—pelvis—thigh—buttock—knee—shin—calf—foot—toes;
   h. hair, nails;
   i. pituitary—duodenum—stomach—gallbladder—intestines—lungs—uterus—bladder—adrenals—parathyroid—liver—pancreas—kidneys—prostate—esophagus—spleen—heart—thymus—worm gland—thyroid—testes—ovaries;
   j. brain;
   k. anatomical parts included in “Terminologia Anatomica The Second Edition”, Parts I-V, Chapters 1-16, which is the international standard for human anatomical terminology;
H. it is a medical condition in which the body is in the state or age of:
   a. embryo
   b. fetus
   C. infantile age
   d. childhood
   e. adulthood
   f. old age
   g. pregnancy and childbirth
   h. disability
   i. coma
   j. anesthesia
   k. sleep
   l. during medical procedures, diagnostics and surgeries;
Terms described in the scientific and patent literature may be used to define the terms of the invention more broadly.

### II. COMPOUNDS OF THE PRESENT INVENTION

Reference will now be made in detail to certain embodiments of the invention, examples of which are shown in the accompanying description, structures, and formulas. Although the invention will be described in connection with the embodiments listed, it will be understood that they are not intended to limit the invention to these embodiments. Rather, the invention is intended to encompass all alternatives, modifications, and equivalents that may be included within the scope of the present invention.

The compounds of the present invention may also exist in the form of physiologically acceptable salts. Examples of physiologically acceptable salts include salts derived from an appropriate base such as an alkali metal or alkaline earth metal (e.g., Na+, K+, Ca+2, Mg+2, Li+), ammonium and NR4+ (where R is defined herein). Physiologically acceptable salts of a nitrogen atom or amino group include (a) acid accession salts formed with inorganic acids, such as hydrochloric acid, hydrobromic acid, sulfuric acid, sulfamic acid, phosphoric acid, nitric acid and the like; (b) salts formed by organic acids, such as, for example, acetic acidtartaric acid, maleic acid, fumaric acid, lysine, arginine, glutamic acid, glycine, serine, threonine, alanine, iso-leucine, leucine and the like, oxalic acid, salicylic acid, gluconic acid, citric acid, malic acid ascorbic acid, benzoic acid, succinic acid, isethionic acid, lactobionic acid, tannic acid, palmitic acid, alginic acid, malonic acid, naphthalenesulfonic acid, methanesulfonic acid, p-toluenesulfonic acid, naphthalenedisulfonic acid, polygalacturonic acid, sulfosalicylic acid, glycolic acid, 2-hydroxy-3-naphthoate, pamoate, stearic acid, phthalic acid, mandelic acid, lactic acid, ethanesulfonic acid; and (c) salts formed from element anions, e.g., chlorine, bromine, and iodine. Physiologically acceptable salts of a hydroxy group compound include the anion of said compound in combination with a suitable cation such as Na+ and NR4+. All such forms are contemplated within the scope of the invention.

Compounds of the invention have the ability to exist as various polymorphs or pseudopolymorphs. As used in this document, crystalline polymorphism refers to the ability of a crystalline compound to exist in different crystal structures. Crystalline polymorphism can result from differences in crystal packing (packing polymorphism) or from differences in packing between different conformers of the same molecule (conformational polymorphism). As used herein, crystalline pseudopolymorphism refers to the ability of a compound hydrate or solvate to exist in different crystal structures. Pseudopolymorphs of the present invention may exist because of differences in crystal packing (packing pseudopolymorphism) or because of differences in packing between different conformers of the same molecule (conformational pseudopolymorphism). All such forms are considered to be within the scope of the invention.

The compounds of the invention can also exist as amorphous solids. As used herein, an amorphous solid is a solid in which there is no long-range ordering of atoms in the solid. This definition also applies when the crystal size is two nanometers or less. Additives, including solvents, can be used to create the amorphous forms of the present invention. All such forms are contemplated within the scope of the invention.

For therapeutic use, the active ingredients of the compounds of the invention will be physiologically acceptable. Ingredients that are not physiologically acceptable, however, may also find use, such as in the preparation or purification of a physiologically acceptable compound.

Lastly, it is to be expected that the compositions presented herein include the compounds of the invention in their non-ionized as well as zwitterionic forms, and combinations with stoichiometric amounts of water, as in hydrates. All such forms are contemplated within the scope of the invention.

It should be noted that all enantiomers, diastereomers and racemic mixtures, tautomers, polymorphs, pseudopolymorphs of compounds are included in the scope of the present invention. All mixtures of such enantiomers and diastereomers are included in the scope of the present invention.

The compounds of the invention can have chiral centers, such as chiral carbon or phosphorus atoms. Thus, the compounds of the invention include racemic mixtures of all stereoisomers, including enantiomers, diastereomers, and atropisomers. Additionally, the compounds of the invention include enriched or resolved optical isomers on any or all asymmetric chiral atoms. Other words, the chiral centers evident from the figures are presented as chiral isomers or racemic mixtures. Racemic and diastereomeric mixtures as well as individual optical isomers isolated or synthesized essentially free from their enantiomeric or diastereomeric partners are within the scope of the invention. Racemic mixtures are divided into their individual, essentially optically pure isomers by well-known methods, such as, for example, separation of diastereomeric salts formed with optically active excipients, such as acids or bases, followed by conversion back to optically active substances. In most cases, the desired optical isomer is synthesized by stereospecific reactions starting from the corresponding stereoisomer of the desired starting substance. All such forms are provided within the scope of the invention.

The compounds of the invention may in some cases exist as tautomeric isomers. While only one delocalized resonance structure may be depicted, all such forms are considered within the scope of the invention.

Any formula or structure herein is also intended to represent unlabeled forms and isotopically labeled forms of compounds. Isotopically labeled compounds have the structures represented in the formulas provided herein except that one or more atoms are replaced with an atom of a selected atomic mass or mass number. Examples of isotopes that may be included in the compounds of the disclosure include isotopes of hydrogen, carbon, nitrogen, oxygen, phosphorus, fluorine and chlorine such as 2H (deuterium, D), 3H (tritium), SS, 13C, 14C, 1SN, 18F, 31P, 32P, 35S, 36C1 and 125I, but without limitation. Different isotopically labeled compounds of the present disclosure, such as those incorporating radioactive isotopes such as 3H, 13C and 14 C. All such forms are provided within the scope of the invention. Such isotopically labeled compounds can be useful in metabolic studies, reaction kinetic studies, detection or imaging techniques such as positron emission tomography (PET) or single photon emission computed tomography (SPECT), including tissue distribution analyses of drugs or substrates, or in the radioactive treatment of patients. The disclosure also includes compounds of Formula I in which 1 to n hydrogens attached to the carbon atom are substituted with deuterium, where n is the number of hydrogens in the molecule. These compounds exhibit increased resistance to metabolism and thus are useful for increasing the half-life of any compound of formula I when administered to a mammal, particularly a human.

The compounds of the present invention may also exist in the form which described in the scientific and patent literature.

### III. KEY METHODS AND CONCEPTS OF THE PRESENT INVENTION

A key part of the invention is to modify the properties of API by creating an API-carrier system (non-exclusive examples of such systems include: solid dispersion of API-carrier, solution of API-carrier, emulsion (microemulsion) of API-carrier (when API and carrier are in co- or different phases), gel of API-carrier, non-covalent complex of API-carrier, inclusion complex of API-carrier, supramolecular complex of API-carrier, conjugate of API-carrier, etc. in which occurs:
a. increasing the solubility of the API by homogenizing and dispersing it in the carrier;
b. creation or strengthening of non-covalent interaction and/or intercalation between API and carrier, including by organizing additional (excessive) non-covalent interaction (including non-equilibrium) or creating supramolecular or inclusion complex bonds;
c. organization of covalent interaction between API and carrier—conjugation of carrier API;
d. increasing the permeability of the API-carrier system due to one of several possible mechanisms;
and associated changes in biophysical, pharmacological, toxicological and therapeutic properties of the API-carrier system compared to API, including due to formation of new non-covalent or conjugated molecular structures between API and carrier.

Carriers plays major role in formulation of solid dispersion. They can be hydrophilic or hydrophobic or water swellable. Depending on their characteristics they can be used as release retardant or release enhancers. Also the dissolution characteristics of drug molecules are depend on nature of carriers. The criteria for selection of best carrier:
a. It should be water soluble or swellable, soluble in variety of solvents.
b. It should be economical, pharmacologically inert, non-toxic.
c. It should be heat stable.
d. Chemically compatible with drug.
e. Water soluble and non-water soluble carriers must prevent the recrystallization trend and help to maintain the supersaturation state after dissolution (spring and parachute effect) via hydrogen bonding interaction
f. It should form a non-viscous solution, as the most commonly used polymers tend to form viscous solutions after dissolution in water, which delays the release of the drug from their matrix. Moreover, these polymers are metabolized in the gastrointestinal tract and release the drug primarily in the colon (Sinh V. R. et al., 2001).

Polymers those are derived from plant origin are used in the current invention because of their diverse pharmaceutical applications and also easy availability, biocompatibility, non-toxic nature, chemically inertness they are preferred over the synthetic ones. Polysaccharide, one of the most abundant industrial raw materials are used because of their sustainability, bio-safety and bio-degradability. The natural gums are metabolic by-products of plants obtained from various parts of plant like seed, fruit, incised trunk (gummy exudate), etc. Natural polysaccharides are biodegradable, biocompatible materials for use in drug delivery systems. However, these materials have certain limitations, like uncontrolled rate of hydration, change in viscosity during shelf life, microbial contamination. Therefore to overcome this problems some modification have done. Modifications can be done in terms of physical modifications and chemical modifications.

### Molecular/Solid Dispersions (SD)

Molecular/Solid dispersion of the invention can be defined as the dispersion of one or more active ingredients in an inert carrier or matrix at solid state prepared by the melting/fusion, solvent, melting-solvent method, mechanochemical treatment and other methods. The drug is hydrophobic in nature whereas matrix is hydrophilic. Solid dispersions of the present invention may be in the form of molecular dispersions, simple eutectic mixtures, solid solutions, glass solutions and glass suspensions, mechanochemically obtained solid dispersions (including solid dispersions in the form of micro-mechano-composites in which solid-phase fusion, mechanosynthesis, mechanoactivation processes have occurred), amorphous precipitation in a crystalline carrier, compound or complex formations. Solid dispersions of the present invention may be in the form of first generation solid dispersions; second generation solid dispersions; third generation solid dispersions; fourth generation solid dispersions. Solid dispersions of the present invention may be in the form of: Class C-C, Class C-A, Class A-C, Class A-A, Class M-C, Class M-A based on physical state and molecular arrangement of both API and carrier. Molecular/Solid dispersions of the present inventions includes inclusion complexes and supramolecular complexes (that can present in solid phase) that can present in form of solid solution in which molecules of one compound (API, carrier) occupy places in the crystal lattice of another compound (API, carrier) or in which one compound (carrier) spatially encloses another (API). Thus in solid dispersions of the invention not only mixing at the molecular level but also complexation can take place.

TABLE 3
Classification According to Physical State
and Molecular Arrangement of API and

| Class | API (active pharmaceutical ingredient) | Carrier |
|---|---|---|
| C-C | Crystalline | Crystalline |
| C-A | Crystalline | Amorphous |
| A-C | Amorphous | Crystalline |
| A-A | Amorphous | Amorphous |
| M-C | Molecularly dispersed | Crystalline |
| M-A | Molecularly dispersed | Amorphous |

Many analytical and instrumental technique are used to characterize solid dispersions. Techniques used for characterization are can be thermal methods, spectroscopic methods, microscopic methods, microthermal analysis, macroscopic techniques, etc. Just a mixture of substances, without treatment of the active pharmaceutical ingredient and carrier, does not lead to the results that solid dispersion leads to. Solid dispersion is not a mixture of synergists that amplify each other's effects. Solid dispersion is not a type of nanoparticle.

The present invention proposes to destroy the crystallinity of the phase, the phase itself and the phase boundaries of API and to form chemical bonds (including hydrogen bonding, van der Waals bonding, donor-acceptor bonding, π-π bonding, Ion-dipole bonding and ion-induced dipole bonding, ionic bonding, hydrophobic bonding and others) between API molecules and the carrier or by creating supramolecular or inclusion complex bonds between API molecules and the carrier and thereby change the properties of API by forming new compounds from it. A number of methods are used for this, generally referred to as “Solid Dispersion Methods” and subdivided into a number of sub-methods, all of which involve molecular mixing and disordered phase processes of the components at the molecular level and create a homogeneous/highly dispersed phase with many chemical interactions between the API and the carrier. All of these processes are described in the literature.

The methods for obtaining molecular/solid dispersions of the present invention include 1) solvent evaporation and cryogenic methods; 2) melting methods and hot-stage extrusion methods; 3) mechanochemical methods (but not excluding other methods). Particle size of solid dispersion may vary and depends on the equipment device (e.g. nozzle size), equipment operation parameters, the degree of grinding of the finished product. The usual particle size is 0.5-1000 microns, but can exceed 1000 microns, and the resulting solid dispersion can also be ground to submicron size after it is obtained.

The solvent evaporation method consists of the solubilization of the drug and carrier in a volatile solvent that is later evaporated. In this method, the thermal decomposition of drugs or carriers can be prevented, since organic solvent evaporation occurs at low temperature. Differences in solvent evaporation processes are related to the solvent evaporation procedure, which usually include vacuum drying, heating of the mixture on a hot plate, slow evaporation of the solvent at low temperature, the use of a rotary evaporator, a stream of nitrogen, spray-drying, freeze-drying, the use of supercritical fluids (SCF) and other types of solvent evaporation.

Melting method consisting of melting the drug within the carrier followed by cooling and pulverization of the obtained product. In the melting process, the molecular mobility of carrier is high enough to change the drug's incorporation. A common adaptation to the melting phase consists of suspending the active drug in a previously melted carrier, instead of using both drug and carrier in the melted state, reducing, therefore, the process temperature. To cool and solidify the melted mixture, several processes such as ice bath agitation, stainless steel thin layer spreading followed by a cold draught, solidification on petri dishes at room temperature inside a dessicator, spreading on plates placed over dry ice, immersion in liquid nitrogen or stored in a dessicator, and other types of cooling were used. After cooling, the mixture must be pulverized regarding its handling. However, the use of high temperatures, and the fact that several drugs can be degraded by the melting process, can be a limitation of this method. The incomplete miscibility between drug and carrier that may occur, because of the high viscosity of a polymeric carrier in the molten state, is another limitation of this process. To avoid the melting method limitations, several modifications, like hot-stage extrusion, Meltrex, melt agglomeration or other were introduced to the original method.

The mechanochemical method of obtaining molecular/solid dispersions consists in intensive mechanical processing (for example, in planetary or other types of mills) under conditions where the processes of solid-phase fusion, mechanosynthesis, mechanoactivation occur between the components, including the formation of microcomposites, in which the processes of mechanochemical solid-phase fusion/melting of the guest substance with the host substance, mechanosynthesis, mechanoactivation have occurred, when the mixing of components results in a reaction in the solid phase, forming products that have high contact surface and extremely high concentration of defects of various kinds. The formation of the interfacial surface, which is necessary for the realization of physic-chemical interaction, takes place. Under these conditions, the characteristic time of mass transfer of reagents to each other for the passage of the physio-chemical reaction decreases. Simultaneously with comminution and formation of mechanochemically obtained solid dispersions, additional structural defects are created in the mixture components. In the macroscopic approximation, the activation barrier of chemical interaction decreases. As a rule, the longer the mechanical processing time, the higher the bioavailability and permeability of the resulting solid dispersions, while the particle size can remain practically unchanged.

At the initial stage of mechanochemical processing the initial collisions of powder particles with balls lead to plastic deformation of particles and their flattening. Continued plastic deformation is accompanied by an increase in the ratio of the surface area of the particles to their volume. In the process of deformation of the particles the pure inner layers of the active pharmaceutical ingredient are opened, which come into close contact with the pure layers of the other component, causing the process of welding. The accompanying plastic deformation hardening reaches a critical value and the formed object is destroyed. The three processes—plastic deformation, welding and fracture of the machined particles—are then repeated many times in parallel. In the process, the material acquires a layered, twisted structure. At the end of the initial fusion stage, a mixed heterophase system called a mechanocomposite is formed. The mechanocomposite has a morphologically metastable structure with a high density of interphase boundaries between the initial components, which provide a developed contact surface and a high concentration of defects due to the large number of atoms on the surfaces and in the near-surface layers. Such a system has a large stored energy, which, together with the extremely large contact surface between the components, provides a high reactivity of the system. At the same time, despite the almost perfect contact surface of the reagents in the matrix, large inclusions of the more brittle component in contact with the plastic reagent can be observed in the volume of the mechanocomposite.

The method of mechanochemical processing leads to the same results as the other methods, but excludes toxic solvents or thermal degradation processes of the active pharmaceutical ingredient.

Solid dispersions of the invention include solid phase inclusion complexes and solid-state supramolecular inclusion complexes in which API molecules are intercalated into the cavity.

Co-treating method of solid dispersion of the present invention can be selected from (but not excluding other methods, which should lead to the formation of the bioavailable solid dispersion):
Method 1 (Mechanochemical Method; Grinding/Milling With High Energy Stress): “guest drug” and the “host substance” are milled in a roller, ball, planetary, vibratory, jet, drum, medium-speed, impact-jet, centrifugal countercurrent, rotary, disintegrator, and other types of mills or other grinders with controlled energy stress. The grinding process may produce particles of a size of mainly 0.5 to 1000 microns, which are powders. Grinding should be carried out to such a state that there are changes in the structure and properties of the ground substance so that it has increased bioavailability and permeability and bioavailable highly permeable molecular/solid dispersion or solid phase non-covalent, inclusion, supramolecular complex is formed. Grinding should cause structural changes such as defects, increased deformation, associates. In the grinding process any of the processes of solid-phase fusion, mechanochemical synthesis, mechanosynthesis, mechanoactivation must occur and including the formation of microcomposites in which the processes of mechanochemical solid-phase fusion of the guest substance with the host substance, mechanosynthesis, mechanoactivation have occurred, when the mixing of components occurs reaction in the solid phase, forming products (including solid phase non-covalent, inclusion, supramolecular complexes) that have high contact surface and extremely high concentration of defects of various kinds. The solvent(s) may be added to the grinding process.
Method 2 (Grinding/Milling With High Energy Stress and Solvent; Mechanochemical Method With Solvent): “guest drug” and the “host substance” are wetted with a solvent, the wetted mass is dried and then ground in the same manner as described in Method 1. The list of solvents used is the same as the solvents listed in Method 1;
Method 3 (Media Milling): The solid dispersion is prepared by using high-shear media mills. The milling chamber charged with milling media, solvent, “guest drug”, and “host substance” is rotated at a very high-shear rate under controlled temperatures. The milling medium is composed of glass, zirconium oxide, highly cross-linked polystyrene resin or other standart milling medium. High energy shear forces are generated as a result of the impaction of the milling media with the “guest drug” and “host substance”. The list of solvents used is the same as the solvents listed in Method 1;
Method 4 (Kneading Method): This method is based on impregnating the “host substance” with a little amount of water or hydroalcoholic solutions to convert it into a paste. The «guest drug» is then added to the above paste and kneaded for a specified time. The kneaded mixture is then dried and passed through a sieve if required. On a laboratory scale, kneading can be achieved by using a mortar and pestle. On large scale, kneading can be done by utilizing extruders and other machines;
Method 5 (Hot-Melt Method, Melting Method, Fusion Method): In this method, the physical mixture of «guest drug» and a “host substance” are heated directly until the two melt. The melted mixture is then cooled and solidified rapidly with rigorous stirring. The final solid mass is then crushed, pulverized, and sieved. The melting method consists of melting the «guest drug» within the “host substance” followed by cooling and pulverization of the obtained product. A common adaptation to the melting phase consists of suspending the «guest drug» in a previously melted “host substance”, instead of using both ((guest drug and “host substance” in the melted state, reducing, therefore, the process temperature. To cool and solidify the melted mixture, several processes such as ice bath agitation, stainless steel thin layer spreading followed by a cold draught, solidification on petri dishes at room temperature inside a desiccator, spreading on plates placed over dry ice, immersion in liquid nitrogen or stored in a desiccators or other cooling method were used. After cooling, the mixture must be pulverized regarding its handling. KinetiSol® dispersing technology also can be used;
Method 6 (Hot-Melt Extrusion, Hot-stage extrusion): Hot-Melt Extrusion is a modification of the Melting Method. Hot-melt extrusion is essentially the same as the melting method except that intense mixing of the components is induced by the extruder. Hot-stage extrusion consists of the extrusion, at high rotational speed, of the «guest drug» and “host substance”, previously mixed, at melting temperature for a small period of time. The resulting product is then collected after cooling at room temperature and milled. A reduction in processing temperature can be achieved by the association of hot-stage extrusion with the use of carbon dioxide as a plasticizer;
Method 7 (Meltrex Method): Meltrex is a modification of the Melting Method. Meltrex is a patented solid dispersion manufacturing process, also based on the melting process. The crucial elements in the Meltrex technology are the use of a special twin screw extruder and the presence of two independent hoppers in which the temperature can vary over a broad temperature range;
Method 8 (Melt agglomeration Method): Melt agglomeration is a modification of the Melting Method. Melt agglomeration allows the preparation of solid dispersions in conventional high shear mixers. It is made by adding the molten “host substance” containing the «guest drug» to the heated excipients, by adding the molten “host substance” to a heated mixture of «guest drug and excipients, or by heating a mixture of the «guest drug, “host substance” and excipients to a temperature within or above the melting range of the “host substance”. It is also possible to produce stable solid dispersions by melt agglomeration in a rotary processor;
Method 9 (High-Pressure Homogenization): In this method, the suspension of an «guest drug and “host substance” is forced under pressure through a micro- or submicro-sized aperture valve of a high-pressure homogenizer;
Method 10 (Solvent Evaporation Method): In this method dissolved both the «guest drug» and the “host substance” in a common solvent and then evaporate the solvent to produce a solid solution. A basic process of preparing solid dispersions of this type consists of dissolving the «guest drug» and the polymeric “host substance” in a common solvent or solvents mixture (the list of solvents used is the same as the solvents listed in Method 1). Normally, the resulting films are pulverized and/or milled. The use of the “host substances” partially suspended, instead of dissolved, was also can be used. Solvent evaporation procedure usually include vacuum drying, heating of the mixture on a hot plate, slow evaporation of the solvent at low temperature, the use of a rotary evaporator, a stream of nitrogen, or other drying procedure;
Method 11 (Spin-coated films Method): Spin-coated films method is a modification of the Solvent Evaporation Method, which consists of dissolving «guest drug» and “host substance” in a common solvent that is dropped onto a clean substrate highly spinned. The solvent is evaporated during spinning;
Method 12 (Spray-drying Method): Spray-drying is a modification of the Solvent Evaporation Method. It consists of dissolving or suspending the «guest drug» and “host substance”, then spraying it into a stream of heated airflow to remove the solvent;
Method 13 (Supercritical Fluid (SCF) Process): Supercritical Fluid Process is a modification of the Solvent Evaporation Method. In this method, the «guest drug» particles are solubilized within the SCF (usually carbon dioxide). Several methods of SCF processing can be used to address individual aspects, such as precipitation with a compressed antisolvent process (PCA), solution enhanced dispersion by SCF (SEDS), supercritical antisolvent processes (SAS), the rapid expansion of supercritical solutions (RESS), gas anti-solvent recrystallization (GAS), and aerosol supercritical extraction system (ASES). In this method modification, the technique consists of dissolving the «guest drug» and the “host substance” in a common solvent that is introduced into a particle formation vessel through a nozzle, simultaneously with CO2. When the solution is sprayed, the solvent is rapidly extracted by the SCF, resulting in the precipitation of solid dispersion particles on the walls and bottom of the vessel;
Method 14 (Cryogenic Techniques): Cryogenic methods are methods in which a solution of «guest drug» and “host substance” in a common solvent is frozen before a low-temperature solvent evaporation procedure. The common solvent can be the same as in the Solvent Evaporation Method. Freezing of the solution can occur after injection. The type of injection device can be a capillary, rotary, pneumatic, ultrasonic nozzle, or other types of the injection device. The location of the nozzle can be above or under the cryogenic liquid level. The composition of cryogenic liquid can be hydrofluoroalkanes, fluorocarbons, N2, Ar, O2, organic solvents, or other types of cryogenic liquid. After cryogenic processing, dry powder can be obtained by various drying processes like spray freeze drying, atmospheric freeze drying, vacuum freeze drying, lyophilization, or other low-temperature drying processes. The list of common solvents used is the same as the solvents listed in Method 1;
Method 15 (Lyophilization/Freeze-Drying Technique): Lyophilization Method is a modification of the Cryogenic Techniques. In this technique, the common solvent from the solution is eliminated through a primary freezing and subsequent drying of the solution containing both «guest drug» and “host substance” at reduced pressure. The basic freeze-drying process consists of dissolving the «guest drug» and “host substance” in a common solvent, which is immersed in cryogenic liquid until it is fully frozen. Then, the frozen solution is further lyophilized;
Method 16 (Spray Freezing onto Cryogenic Fluids): This Method is a modification of the Cryogenic Techniques. In this technique, the «guest drug» and the “host substance” were dissolved in a common solvent and atomized above the surface of a boiling agitated cryogenic liquid refrigerant;
Method 17 (Spray Freezing into Cryogenic Liquids (SFL)): This Method is a modification of the Cryogenic Techniques. It incorporates direct liquid-liquid impingement (by spraying) between the solution and cryogenic liquid to provide intense atomization into microdroplets. The frozen particles are then lyophilized to obtain dry and free-flowing micronized powders.
Method 18 (Spray Freezing into Vapor over Liquid (SFV/L)): This Method is a modification of the Cryogenic Techniques. Freezing of «guest drug» solutions in cryogenic fluid vapors and subsequent removal of frozen solvent. During SFV/L the atomized droplets typically start to freeze in the vapor phase before they contact the cryogenic liquid. As the solvent freezes, the «guest drug» becomes supersaturated in the unfrozen regions of the atomized droplet.
Method 19 (Ultra-Rapid Freezing): This Method is a modification of the Cryogenic Techniques. Application of «guest drug» solution to the solid surface of the cryogenic substrate leads to instantaneous freezing and subsequent lyophilization.
Method 20 (Precipitation Technique, Co-precipitation Method): In the precipitation method the «guest drug» is dissolved in a solvent, which is then added to Non-solvent to precipitate the crystals. Non-solvent is added dropwise to the «guest drug» and “host substance” solution, under constant stirring. In the course of the non-solvent addition, the «guest drug» and “host substance” are co-precipitated to form micro-particles. In the end, the resulted micro-particle suspension is filtered and dried. The list of solvents used is the same as the solvents listed in Method 1.
Method 21 (Microwave Irradiation Method): This method involves the microwave irradiation reaction between «guest drug» and complexing agent using a microwave oven. The «guest drug and “host substance” in a definite molar ratio are dissolved in a mixture of water and/or organic solvent in a specified proportion into a container. The mixture is reacted in the microwave oven. After the reaction completes, an adequate amount of solvent mixture is added to the above reaction mixture to remove the residual uncomplexed free «guest drug and “host substance”. The precipitate so obtained is separated using a filter, and dried. The list of solvents used is the same as the solvents listed in Method 1.
Method 22 (Energy Input Method): Method comprises the step of exposing a host substance and a «guest drug» to an energy input until a bioavailable highly permeable solid dispersion is formed of the «guest drug» as amorphous material entrapped in the host substance.
Method 23 (Heat/Shear Energy Input Method): Method comprises the step of exposing a host substance and a «guest drug» to an energy input, whereby the energy input is heat and/or shear forces, until a bioavailable highly permeable solid dispersion is formed of the «guest drug» as amorphous material entrapped in the host substance.
Method 24: Other methods—electrostatic spinning, electrostatic blowing, electrospraying film casting; gel entrapment technique, KinetiSol dispersing (KSD), drum drying, freeze-drying (FD), rotary evaporation (RE), spray congealing, co-precipitation (CP), co-grinding, spin-coated film (SCF), centrifuge vacuum drying (CVD), melt cooling and the like;
Method 25: A method comprising—contacting an active pharmaceutical ingredient and a matrix “host substance” to form a solid dispersion under conditions sufficient to form a solid dispersion;
Method 26 (Combined method): This method includes any combination of Methods 1-25.
Methods 1-26 should lead to the formation of the bioavailable solid dispersion.

Others methods of obtaining of SD are described in the scientific and patent literature.

### Non-Covalent Complexes (NCC)

Non-covalent complex of the invention are molecular ensembles that, as a whole, have specific properties that differ from the environment and whose internal structure and form are determined by non-covalent interactions of atoms. This distinguishes noncovalent systems from molecular systems in which the structure is determined mainly by covalent interactions. Noncovalent complex are molecular complexes of two or more cationic, anionic or neutral molecules held together in a single system by hydrogen bonding, van der Waals interactions, donor-acceptor interactions, π-π interactions, ionic interactions, hydrophobic interactions. Noncovalent complex have the ability to form different architectures while the chemical composition of the system remains unchanged. Noncovalent complex of the invention consisting of at least two different components, generally a matrix (“host”, “host substance”, “carrier”, etc.) and a therapeutic agent (API, active pharmaceutical ingredient, drug, guest, “guest drug”, “guest drug compound”, “guest substance”, etc.). Many analytical and instrumental technique are used to characterize noncovalent complexes (spectroscopic methods, etc.). Noncovalent complexes play a particularly important role in biological systems, such as the transfer of molecules and ions across biological membranes. The rate of release of active pharmaceutical ingredient molecules from noncovalent complexes or conjugate can play a significant role in increasing bioavailability.

Non-covalent complexes of the invention include inclusion complexes in which API molecules are intercalated into the cavity, host-guest complexes, and supramolecular complexes.

Method of preparation of non-covalent active pharmaceutical ingredient-carrier complex of the present invention is selected from (but not excluding other methods, which should lead to the formation of the bioavailable non-covalent complex and which are described in the scientific and patent literature):
a) Dissolving solid dispersion in solvent (usually water) to form the liquid solubilized noncovalent complex (to form solutions, gels, colloids, sols, suspensions, etc.) under conditions sufficient to form the liquid solubilized noncovalent complex;
b) Liquid-phase method for obtaining a complex of carrier with API: a pre-prepared aqueous solution of the corresponding starting carrier and a solution of API in an organic solvent miscible with water, such as acetone, are mixed under stirring and heating to a temperature not exceeding 70° C., followed by holding under stirring at said temperature until a homogeneous solution is obtained with isolation of the obtained complex, possibly in the form of a crystalline solid suspension, which, if necessary, is further ground to obtain the product;
c) Dissolving host substance and guest drug in solvent (usually ethanol or acetone) to form the liquid solubilized noncovalent complex;
d) Host substance is placed in a flask and completely dissolved in ethanol. Then a guest drug is added to the flask. Water is slowly added to the solution and stirred for several hours;
e) (a-d) when energy is supplied by any other means, such as (but not limited to)electromagnetic radiation, heat, mechanical action, sound, ultrasound, or in any of the ways described in the scientific and patent literature. As a non-limiting example, an example of exposure by mechanical and acoustic influence is given. Or non-covalent complex obtained by the interaction of aqueous or non-aqueous solutions of host substance and guest drug under conditions of mechano-acoustic and precipitation of the resulting complex with ethyl alcohol.

Others methods of obtaining of NCC are described in the scientific and patent literature.

### Conjugates (CON)

Another approach of the invention to improving drug performance and reducing toxicity is the conjugation to a polymeric carrier. Chemical conjugation of API to a water-soluble, biodegradable, and biocompatible polymer could present many advantages over presently available API formulations. Conjugation of an insoluble drug to a water-soluble biodegradable polymer may increase the water solubility of the drug, drug circulation time, and accumulation in the diseased tissue, resulting in an improved therapeutic effect and reduced toxicity.

Polysaccharides have high chemical stability. Because of the stability of polysaccharides, covalent linkages between therapeutic agents and polysaccharides can be achieved in organic solvents. This is a considerable advantage since some therapeutic agents have low water solubility. Working in nonaqueous media, water-labile linkages (e.g., esters) can be created between the therapeutic agent and the polysaccharide. In addition, polysaccharides do not denature at high temperature, and tolerate extremes of pH or in organic solvents.

Methods of preparation of conjugate of active pharmaceutical ingredient and carrier is selected from (but not excluding other methods, which should lead to the formation of the bioavailable CON):
Method 1. API was conjugated to oxidized carrier in borate buffer solution, pH ~11, for 2-48 h at room temperature. Pure water-soluble conjugates were obtained as lyophilized powder. The reduced conjugate where the drug is attached by a stable amine bond can be prepared by reducing the imine bond of the derivative using sodium borohydride. API was conjugated carrier in two steps: first, carrier was oxidized to dialdehyde carrier with potassium periodate in water at 25° C. and purified from the interfering oxidative anions (periodate and iodate) by passage through an ion-exchange column of Dowex-1 (Aldrich, Milwaukee, Wis.) in acetate form. Dowex-1 acetate was obtained by pretreating the commercial anion exchanger with 1 M acetic acid. The purified oxidized carrier solution was dialyzed through a 12,000-molecular-weight cutoff dialysis tubing (Medicell International, London, United Kingdom) against deionized water for 48 h at 4° C. and lyophilized to dryness. In the next step, API was bound to the oxidized carrier by the formation of an imine bond between the primary amine bond of API and the aldehydes of the oxidized carrier. Conjugation occurred in 0.1 M borate buffer solution, pH 11, for 48 h under constant stirring. The imine conjugate was purified by dialysis and lyophilized to dryness. The imine (unreduced) conjugate was reduced to a more stable amine form by the addition of sodium borohydride powder (1.1 M NaBH₄/mol of saccharide units in the polymer) to the imine conjugate solution, with overnight stirring at room temperature. The amine (reduced) conjugate obtained was purified by dialysis and lyophylized as described above for the imine conjugate.
Method 2. This method demonstrates the attachment of a API to a hydrolysis product of carrier. Preparation of the carrier Hydrolysis Product. Carrier (100 g) was dissolved in 37 C water to give a 20% solution. In a separate container, NaOH (200 g) was dissolved in 37 C water (5 L). Sodium borohydride (20 g) was dissolved in the NaOH solution, and the carrier solution was then added to this solution. Another 20 g of sodium borohydride was added to the reaction, and the reaction mixture was stirred at 37 C for 15 min, whereupon the mixture was brought to pH 8.6 by the addition of concentrated cold HCl 4° C.
The solution was extensively dialyzed using 3000 molecular weight cut-off dialysis tubing. The dialysate was filtered through an 0.22 micron filter and lyophylized to give the carrier hydrolysis product as a white crystalline solid.
Preparation of Carboxymethyl Carrier Hydrolysis Product. Fifty grams of the carrier hydrolysis product was dissolved in 200 mL of water. To this solution was added one mole of NaOH, followed by bromoacetic acid (70-275 mmols). The reaction was allowed to run for 90 min at 30 C. At the end of the reaction, the solution was neutralized by the addition of 6N HCl. The product was then purified with extensive ultrafiltration using an appropriate filter, then lyophilized.
Attaching the Therapeutic Agent. The coupling of API to the carboxymethyl hydrolysis product of carrier was performed in a two step process. To a mixture of ethylene diamine (2.5 mL), adenosine5-monophosphate (10 g), and 1-hydroxybenzotriazole (HOBt, 3.7Eg) was added 1-ethyl-3-(3-dimethylaminopropyl) carbodiimide (EDC, 15 g) over 3.5 h at 40 C in 4 portions. The water was removed under vacuum and the product triturated with 50 mL of 2:1 CH3CN/MeOH. The solid was washed with an additional 100 mL of acetonitrile and dried under vacuum at 50 C. The solid was passed through a 2.5 cm×25 cm column containing 100 mL of wet volume Amberlite IR-120 (Na form) ion exchange resin. The fractions containing the product, ethylenediamine-APIwere combined and the water was removed under vacuum. The product was again triturated with 2:1 acetone/MeOH, and washed with 100 mL of acetone. The product was dried under vacuum for 2 h at 50 C.

### Bioavailability and Permeability

In the present invention, bioavailable solid dispersions, bioavailable noncovalent complexes and conjugates are obtained and used. Bioavailable with respect to the solid dispersions, noncovalent complexes, or conjugat means at least one of the following:
1) absolute, relative bioavailability of the solid dispersion, noncovalent complex, or conjugate of the active pharmaceutical ingredient is higher than that of the active pharmaceutical ingredient itself;
2) concentrations, Cmax, AUC and other pharmokinetic parameters of active pharmaceutical ingredient and/or its metabolites in blood, plasma, organs, or tissues for the solid dispersion, noncovalent complex, or conjugate of the active pharmaceutical ingredient is higher than that of the active pharmaceutical ingredient itself.

Highly permeable solid dispersions, noncovalent complexes, or conjugates are obtained and used in the current invention. Highly permeable with respect to the solid dispersions, the noncovalent complexes, or conjugate of the present invention means at least one of them:
1) the transfer of molecules, ions, metabolites of active pharmaceutical ingredient across the biological/cell membrane is improved for the solid dispersion or the noncovalent complex or conjugate of active pharmaceutical ingredient than for the active pharmaceutical ingredient itself is presented;
2) the transfer of molecules, ions, metabolites of active pharmaceutical ingredient across the Caco-2 cell line layer or other cell line layer (the intestinal epithelium cells layer, etc.) is improved for the solid dispersion, the noncovalent complex, or conjugate of active pharmaceutical ingredient than for the active pharmaceutical ingredient itself is presented;
3) intestinal permeability is improved for the solid dispersion, the noncovalent complex, or conjugate of active pharmaceutical ingredient than for the active pharmaceutical ingredient itself is presented;
4) reverse transport of API is impeded;
5) metabolism is blocked and API accumulates in tissues;
6) increase in permeability according to the methods used to determine permeability in The Biopharmaceutics Classification System.

Within the scope of the current invention, an increase in permeability, an increase in absorption, increase a concentration of an API in cells, tissues from solid dispersions, noncovalent complexes, or conjugates is achieved through the following mechanisms (but not excluding other mechanisms for increasing permeability):
1) as a consequence of blocking/reducing the activity of CYP3A4 and/or P-glycoproteins, blocking/reducing the activity of other transmembrane carrier proteins, transporters, ABC transporters, enzymes, etc., by membrane interacting with carrier substance molecules (or carrier molecular part of NCC or conjugate);
2) due to blocking/reduction of CYP3A4 and/or P-glycoprotein activity, blocking/reduction of activity of other transmembrane protein transporters, transporters, enzymes, etc., by carrier substance molecules interacting with the membrane due to adhesion of carrier substance molecules (or carrier molecular part of NCC or conjugate) to the cellular layer. Such adhesion can lead to interaction with transporters domains located on the outer part of the cell membrane and block the action of the transmembrane transporter protein;
3) due to a change in the structure of the cell membrane after the incorporation of carrier substance molecules (or carrier molecular part of NCC or conjugate) into this membrane, rather than due to adhesion;
4) due to the direct effect of carrier substance molecules (or carrier molecular part of NCC or conjugate) on the intestinal epithelium cell membrane: it may consist in alteration of lipid bilayer properties, density of intercellular contacts or inhibition of trans-membrane proteins of multidrug resistance;
5) due to changes in hydrophilicity, hydrophobicity, or other biophysical properties of the non-covalent complex, or conjugate due to which changes in the paracellular transport of the non-covalent complex, or conjugates may occur;
6) due to changes in hydrophilicity, hydrophobicity or other biophysical properties of the non-covalent complex, or conjugate due to which changes in the transmembrane transport of the non-covalent complex, or conjugates may occur (passive, active transport);
7) due to adhesion to mucin. Carrier may form sufficiently strong bonds with mucin, which was demonstrated in an in vitro experiment using mucin from pig stomach. When comparing the adhesion index with mucin of several polysaccharides—hyaluronic acid, polysaccharide from tamarind seeds and AG, it was shown that AG has the greatest mucoadhesive property;
8) other changes in the structure and properties of the membrane or other biophysical properties of the non-covalent complex, or conjugate, through other mechanisms that contribute to an increase in its permeability;
9) other mechanism of increase in permeability that are described in the scientific and patent literature.

### Other Possible Formulations to Increase Bioavailability and Permeability

In alternative embodiments, API, AAPI, AANPI can be formulated in any way for the manufacture of drugs with enhanced bioavailability and permeability, solubility, reduced toxicity. (including drug delivery systems) and can be applied in a variety of forms including nanoparticles, microparticles, nanocomposites, microcomposites, composites, polymeric nanoparticles (PNPs), liposomes, freeze-dried liposomes, micelles, polymeric micelles, niosomes, solid lipid nanoparticles (SLNs), nanostructured lipid carriers (NLCs), nanoemulsions, microemulsions, emulsions, self-nanoemulsifying drug delivery systems (SNEDDS), nanocrystals, co-crystals, mesoporous silica nanoparticles (MSNs), dendrimers, ultradispersions, solid dispersions, solid amorphous dispersions, noncovalent complexes, complexes with polymer, complexes with biopolymer, host-guest complexes, inclusion complexes, solutions, solutions in fat/oil, solutions with cosolvent, solutions with surface active agent/surfactant, solutions with solubilizing agent, polymorphes, lipid-based systems, lipid-based formulations, lipid-based drug delivery systems (LBDDS), gels, colloids, sols, or in any form described in the scientific and patent literature for example
or/and API, AAPI, AANPI can be obtained using methods of
   A) Reduction Particle size
      a) Micronization
Physical b) Nanosuspension,
modification B) Modification of the crystal habit
   C) Solid dispersions
      a) Eutectic mixtures
      b) Solid solutions
      c) Amorphous solid solutions
      d) Glass solutions and glass suspension
      e) Cryogenic techniques.
   A) Change of pH,
Chemical B) Use of buffer,
modification C) Derivatization,
   D) Complexation,
   E) Salt formation.
Miscellaneous A) Supercritical fluid process,
methods B) Use of adjuvant like surfactant, solubilizers, cosolvency, hydrotropy, and novel excipients.
and any of the ways described in the scientific and patent literature.

### IV. Non-Covalent Complexes, Solid Dispersions and Conjugates of the Invention

Provided are the non-covalent active pharmaceutical ingredient-carrier complex (of Formula I)

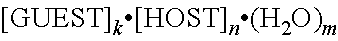

with enhanced permeability and/or bioavailability properties for treating diseases in a human or animal or mammal or bird as well as for manufacturing a pharmaceutical dosage form to treat disease a human or animal or mammal or bird in need thereof (by administering a therapeutically effective amount), at a dosage ranging from 0.0001 ng/kg to 100000 mg/kg (calculated for pure API)
wherein k is 1 to 100; wherein n is 1 to 10000; wherein m is 1 to 100000;
wherein GUEST is
   a)

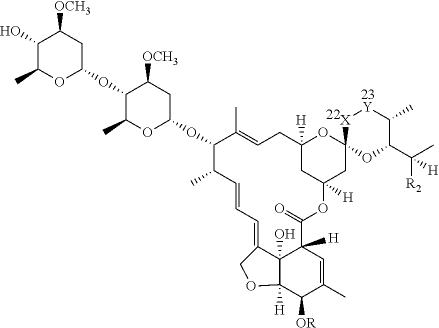

wherein
   1) R₁ is CH₃; R₂ is CH₂CH₃; X—Y is CH═CH; (i.e. GUEST is Avermectin A1a);
   2) R₁ is CH₃; R₂ is CH₃; X—Y is CH═CH; (i.e. GUEST is Avermectin A1b);
   3) R₁ is CH₃; R₂ is CH₂CH₃; X—Y is CH₂—CHOH; (i.e. GUEST is Avermectin A2a);
   4) R₁ is CH₃; R₂ is CH₃; X—Y is CH₂—CHOH; (i.e. GUEST is Avermectin A2b);
   5) R₁ is H; R₂ is CH₂CH₃; X—Y is CH═CH; (i.e. GUEST is Avermectin B1a);
   6) R₁ is H; R₂ is CH₃; X—Y is CH═CH; (i.e. GUEST is Avermectin B1b);
   7) R₁ is H; R₂ is CH₂CH₃; X—Y is CH₂—CHOH; (i.e. GUEST is Avermectin B2a);
   8) R₁ is H; R₂ is CH₃; X—Y is CH₂—CHOH; (i.e. GUEST is Avermectin B2b);
   9) R₁ is H; R₂ is CH₂CH₃; X—Y is CH₂—CH₂; (i.e. GUEST is Ivermectin B1a);
   10) R₁ is H; R₂ is CH₃; X—Y is CH₂—CH₂; (i.e. GUEST is Ivermectin B1b);
   11) R₁ is CH₃; R₂ is CH₂CH₃; X—Y is CH₂—CH₂; (i.e. GUEST is Ivermectin A1a);
   12) R₁ is CH₃; R₂ is CH₃; X—Y is CH2-CH2; (i.e. GUEST is Ivermectin A1b);
b)

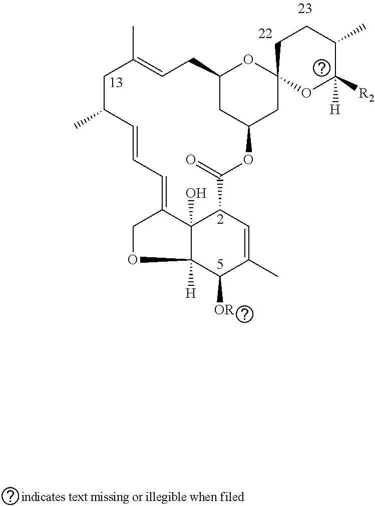

wherein
   1) R₁ is H R₂ is CH₃; (i.e. GUEST is Milbemycin A3);
   2) R₁ is H R₂ is CH₂CH₃; (i.e. GUEST is Milbemycin A4);
   3) R₁ is H R₂ is CH(CH₃)₂; (i.e. GUEST is Milbemycin D);
   4) R₁ is CH₃; R₂ is CH₃; (i.e. GUEST is Milbemycin B2);
   5) R₁ is CH₃; R₂ is CH₂CH₃; (i.e. GUEST is Milbemycin B3);
   6) R₁ is CH₃; R₂ is CH(CH₃)₂; (i.e. GUEST is Milbemycin G);
c)

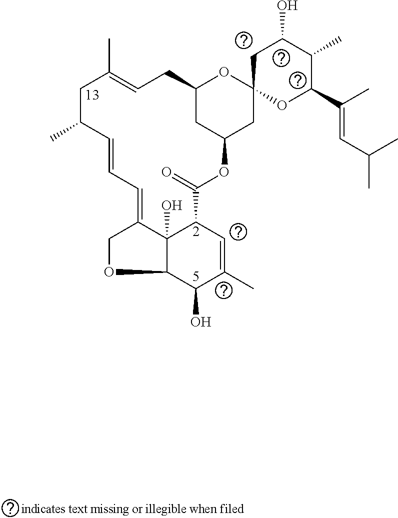 [SMILES: CC1=C[C@H]2C(=O)O[C@H]3C[C@@H](C/C=C(\C)C[C@@H](C)/C=C/C=C4\CO[C@H]([C@@H]1O)[C@@]42O)O[C@]1(C3)C[C@H](O)[C@H](C)[C@@H](/C(C)=C/C(C)C)O1]

(i.e. GUEST is Nemadectin);
d)

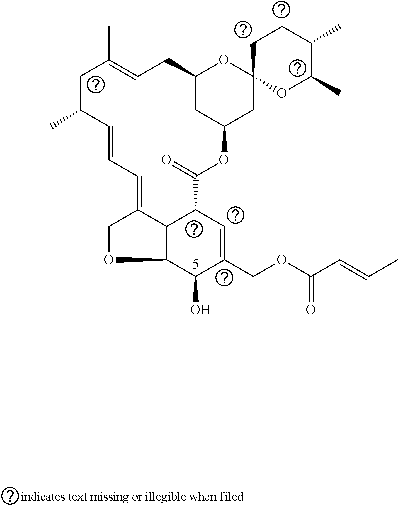

(i.e. GUEST is Meilingmycin A);
e)

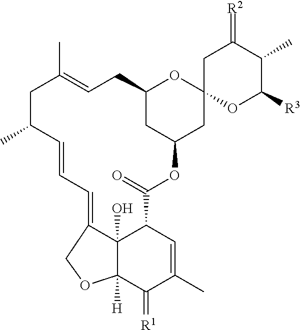

wherein
   1) ═R¹ is —H(β)—OH; ═R₂ is —H, —H; —R³ is selected from —CH₃, —CH₂CH₃; (i.e. GUEST is Milbemectin);
   2) ═R¹ is ═NOH; ═R₂ is —H, —H; —R³ is selected from —CH₃, —CH₂CH₃; (i.e. GUEST is Milbemycin oxime);
   3) ═R¹ is —H, (β)—OH; ═R₂ is ═NOCH₃; —R³ is (Z)—C(CH₃)═CH—CH(CH₃)₂; (i.e. GUEST is Moxidectin);
   4) ═R¹ is —H, (β)—OH; ═R₂ is —H, (α)—OH; —R³ is (Z)—C(CH₃)═CH—CH(CH₃)₂; (i.e. GUEST is Nemadectin);
f)

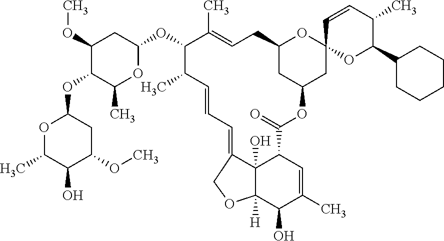 [SMILES: CO[C@H]1C[C@H](O[C@H]2[C@H](C)O[C@@H](O[C@@H]3/C(C)=C/C[C@@H]4C[C@@H](C[C@]5(C=C[C@H](C)[C@@H](C6CCCCC6)O5)O4)OC(=O)[C@@H]4C=C(C)[C@@H](O)[C@H]5OC/C(=C\C=C\[C@@H]3C)[C@]54O)C[C@@H]2OC)O[C@@H](C)[C@@H]1O]

(i.e. GUEST is Doramectin);
g)

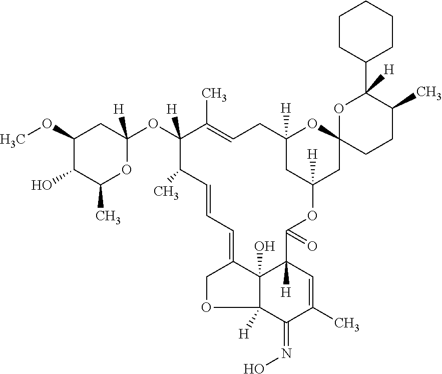

(i.e. GUEST isSelamectin);
h)

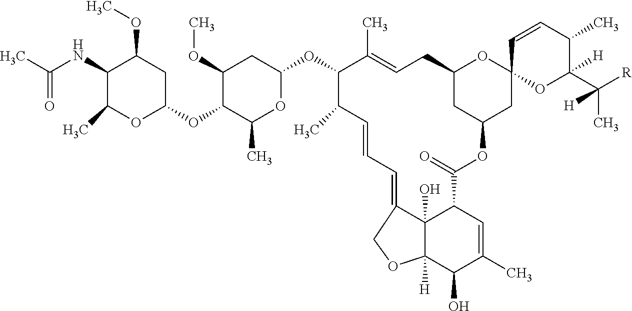

wherein
   1) R is CH2CH3; (i.e. GUEST is Eprinomectin B1a);
   2) R is CH3; (i.e. GUEST is Eprinomectin B1b);
i)

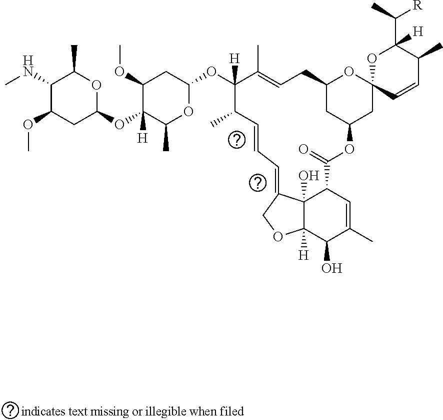

wherein
   1) R is CH2CH3; (i.e. GUEST is Emamectin B1a);
   2) R is CH3; (i.e. GUEST is Emamectin B1b);
j)

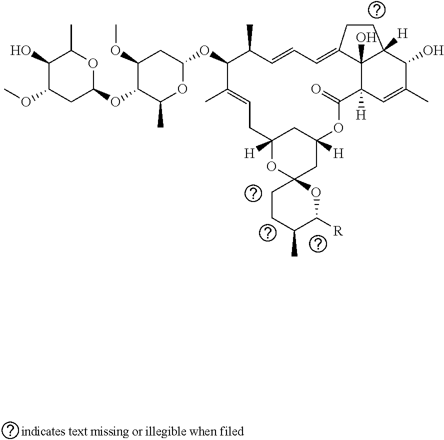

wherein
   1) R is CH2CH3; (i.e. GUEST is TenvermectinB);
   2) R is CH3; (i.e. GUEST is TenvermectinA);
k)

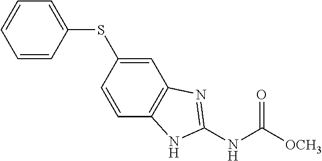 [SMILES: COC(=O)Nc1nc2cc(Sc3ccccc3)ccc2[nH]1]

(i.e. GUEST is Fenbendazole);
l)

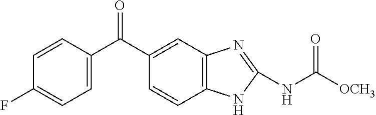 [SMILES: COC(=O)Nc1nc2cc(C(=O)c3ccc(F)cc3)ccc2[nH]1]

(i.e. GUEST is Flubendazole);
m)

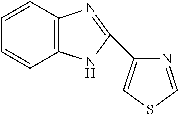 [SMILES: c1ccc2[nH]c(-c3cscn3)nc2c1]

(i e. GUEST is Thiabendazole (Tiabendazole));
n)

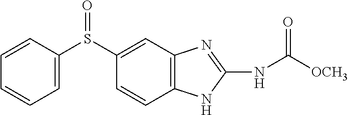 [SMILES: COC(=O)Nc1nc2cc(S(=O)c3ccccc3)ccc2[nH]1]

(i.e. GUEST is Oxfendazole);
o)

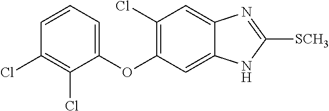 [SMILES: CSc1nc2cc(Cl)c(Oc3cccc(Cl)c3Cl)cc2[nH]1]

(i.e. GUEST is Triclabendazole);
p)

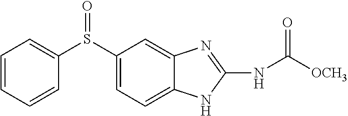 [SMILES: COC(=O)Nc1nc2cc(S(=O)c3ccccc3)ccc2[nH]1]

(i.e. GUEST is Mebendazole);
q)

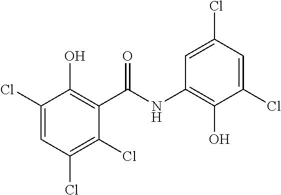 [SMILES: O=C(Nc1cc(Cl)cc(Cl)c1O)c1c(O)c(Cl)cc(Cl)c1Cl]

(i.e. GUEST is Oxyclozanide);
r)

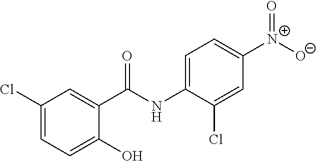 [SMILES: O=C(Nc1ccc([N+](=O)[O-])cc1Cl)c1cc(Cl)ccc1O]

(i.e. GUEST is Niclosamide);
s)

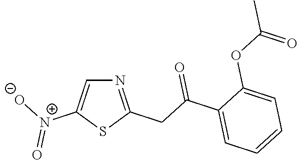 [SMILES: CC(=O)Oc1ccccc1C(=O)Cc1ncc([N+](=O)[O-])s1]

(i.e. GUEST is Nitazoxanide);
t)

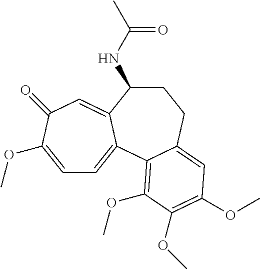

(i.e. GUEST is Colchicine);
u)

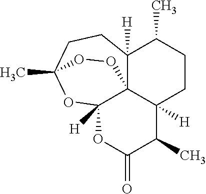

(i.e. GUEST is Artemisinin);
v)

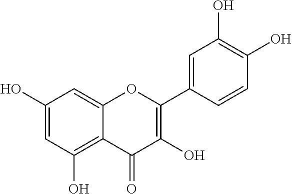 [SMILES: O=c1c(O)c(-c2ccc(O)c(O)c2)oc2cc(O)cc(O)c12]

(i.e. GUEST is Quercetin);
w)

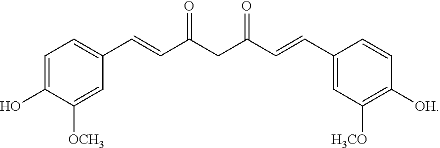 [SMILES: COc1cc(/C=C/C(=O)CC(=O)/C=C/c2ccc(O)c(OC)c2)ccc1O]

(i.e. GUEST is Curcumin);
x)

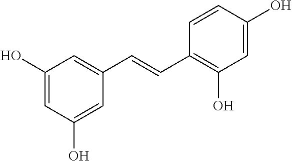 [SMILES: Oc1cc(O)cc(/C=C/c2ccc(O)cc2O)c1]

(i.e. GUEST is Resveratrol);
y)

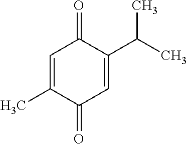

(i.e. GUEST is thymoquinone);
z)

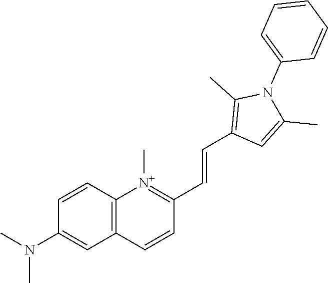 [SMILES: Cc1cc(/C=C/c2ccc3cc(N(C)C)ccc3[n+]2C)c(C)n1-c1ccccc1]

(i.e. GUEST is Pyrvinium);
aa)

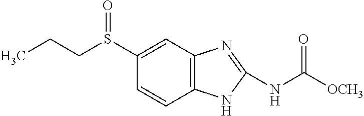 [SMILES: CCCS(=O)c1ccc2[nH]c(NC(=O)OC)nc2c1]

(i.e. GUEST is Albendazole sulfoxide S-oxide));
ab)

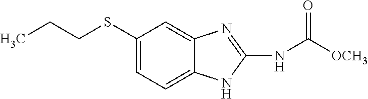 [SMILES: CCCSc1ccc2[nH]c(NC(=O)OC)nc2c1]

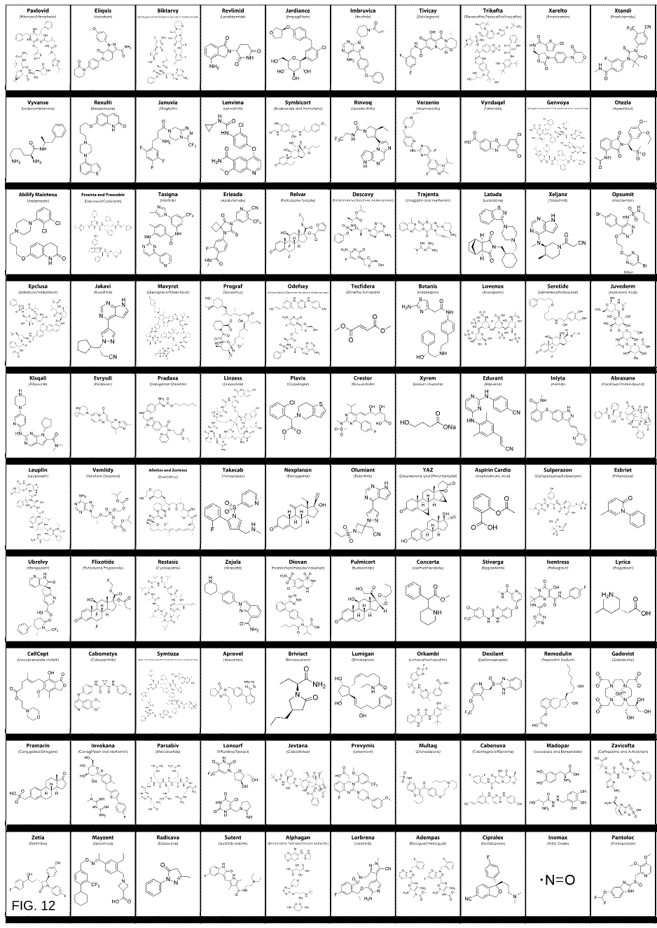

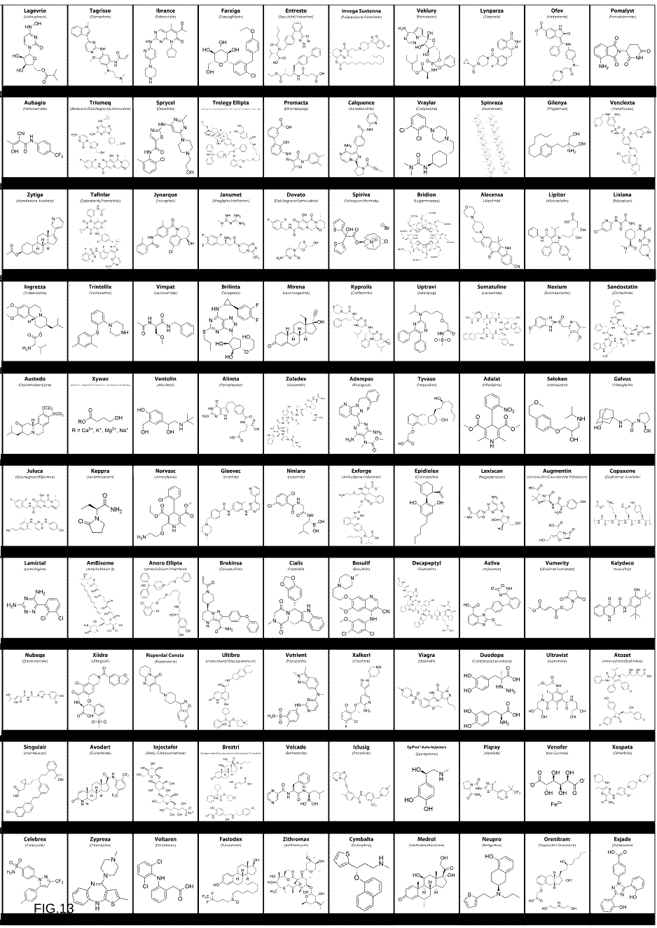

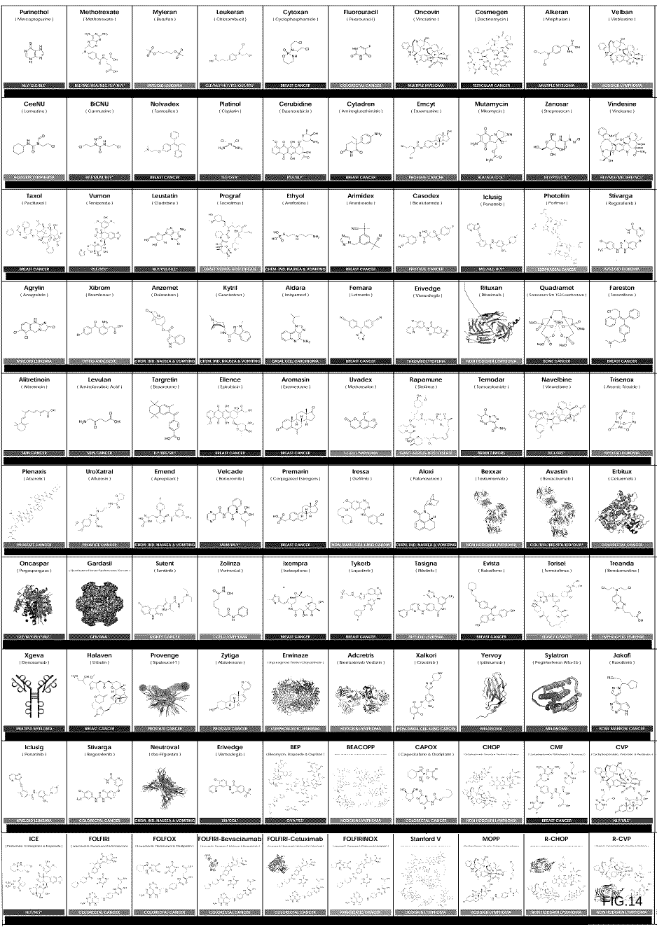

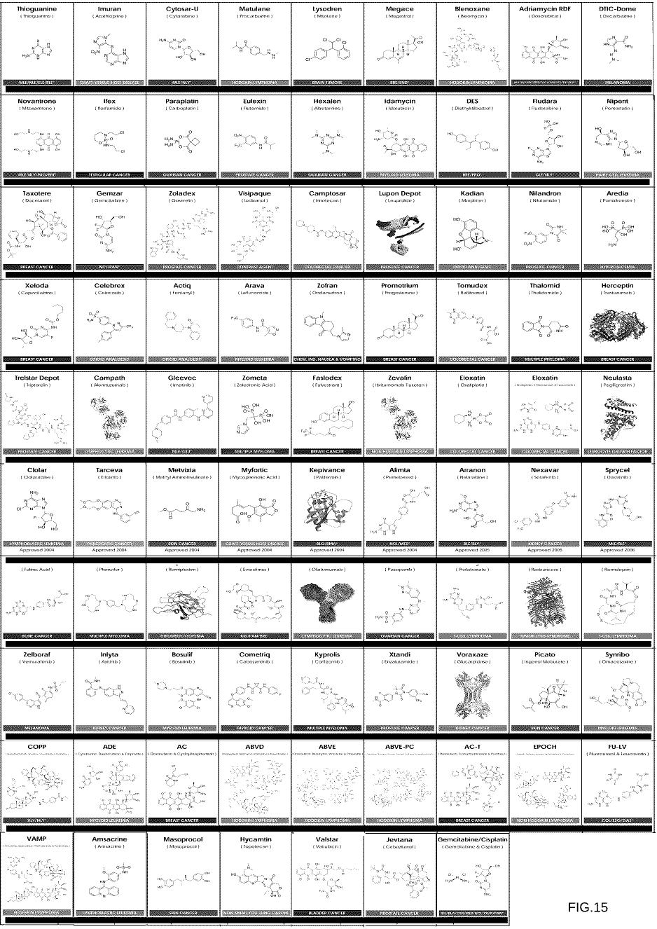

(i.e. GUEST is Albendazole);
ac) GUESTS selected from those listed in from FIG. 12 to FIG. 15.
ad) derivatives of compounds specified in (a)-(ac) such as racemate, enantiomer, diastereomer, tautomer, polymorph, pseudopolymorph, hydride or solvate thereof; or a pharmaceutically acceptable salt or ester or ether thereof; or prodrug thereof; or methabolite thereof; or aglycone thereof; or derivatives thereof (including chemical and physical); or derivatives which may be obtained by gene replacement processes in genetically engineered strains, or/and by fermentation, or/and by chemical modifications and the like, or/and which are described in the scientific and patent literature, for example (non-limited examples) in:

| | |
|---|---|
| methoxime | Li, C., He, H., Wang, J. et al. Characterization of a LAL-type regulator NemR in nemadectin biosynthesis and |
| derivatives | its application for increasing nemadectin production in Streptomyces cyaneogriseus. Sci. China Life Sci. 62, |
| | 394-405 (2019), https//doi.org/10.1007/s11427-018-9442-9 |
| nemadectin | Xang W S., Wung. J D., Wung. M et al. New nemadectin congener from Streptomyces microflavus neau3: |
| congeners | fermentation, isolation, structure elucidation and biological activities. J Antibiot 63, 171-175, (2010) |
| Milbemycin | https//doi.org/10.1038/ja.2010.12 |
| derivatives | Zhang Y., He H., Liu H., Wang H, Wang X, Xiang W. Characterization of a pathway-specific activator of |
| | milbemycin biosynthesis and improved milbemycin production by its overexpression in Streptomyces |
| | bingchenggensis. Microb Cell Fact. 2016 Sep. 7;15(1):152. doi: 10.1186/s12934-016-0552-1. PMID: |
| | 27604457 PMCID: PMC5015266. |
| 5- | Wei K, Wu Y, Li L, Jiang W, Hu J, Lu Y, Chen S. MilR2, a novel TetR family regulator involved in 5- |
| oxomilbemycin | oxomilbemycin A3/A4 biosynthesis in Streptomyces hygroscopicus. Appl Microbiol Biotechnol. 2018 |
| A3/A4 | October; 102(20): 8841-8853. doi: 10.1007/s00253-018-9280-2 Epub. 2018 Aug. 18, PMID: 10121789 |
| derivatives | Satyavan Sharma, Nitya Anand, Chapter 3 - Natural Products, Editor(s): Satyavan Sharma, Nitya Anand, |
| | Pharmacochemistry Library, Elsevier, Volume 25, 1997, Pages 71-123, ISSN 0165-7208, ISBN |
| | 9780444894762, https://doi.org/10.1016/S0165-7208(97)80025-6. |
| | (https://www.sciencedirect.com/science/article/pii/S0165720897800256) |
| tenvermectin | Wan, X., Zhang, Sy., Zhang, H., et al. New tenvermectin analogs obtained by microbial conversion with |
| analogs | Saccharopolyspora erythraea J Antibiot 70, 190-192 (2017), https://doi.org/10.1038/ja.2016.91 |
| tenvermectin | CHEN Hao, ZHANG Shaoyong, HAN Qunqi, ZHANG Hui, WANG Jidong, CHEN Anliang. Synthesis, |
| analogs | insecticidal and acaricidal activities of tenvermectin acylated derivatives[J]. Chinese Journal of Pesticide |
| | Science, 2020, 22(1): 35-47. doi: 10.16801/j.issn.1008-7303.2020.0014 |
| Δ^{2,5}- | Noël, F., Pimenta, P. H. C., dos Santos, A. R. et al. Δ^{2,5}-ivermectin ethyl secoester, a conjugated ivermectin |
| ivermectin | derivative with leishmanicidal activity but without inhibitory effect on mammalian P-type ATPases. Naunyn |
| ethyl | Schmied Arch Pharmacol383. 101-107 (2011), https://doi.org/10.1007/s00210-010-0578-6 |
| secoester | |
| Avermectin | Jun Huang. An-Liang Chen. Hui Zhang. Zhen Yu. Mei-Hong Li. Na Li. Jia-Tan Lin. Hua Bai. Ji-Dong Wang. |
| Derivative | Yu-Guo Zheng. Gene Replacement for the Generation of Designed Novel Avermectin Derivatives with |
| | Enhanced Acaricidal and Nematicidal Activities. DOI: https://doi.org/10.1128/AEM.01025-15 |
| Avermectin | Avermectin Biosynthesis. Haruo Ikeda and Satoshi Omura Chemical Review199797 (7). 2591-2610 DOI: |
| Derivative | 10.1021/cr960023p |
| Avermectin | Awasthi A, Razzak M, Al-Kassas R, Harvey J, Garg S. An overview on chemical derivatization and stability |
| Derivative | aspects of selected avermectin derivatives. Chem Pharm Bull (Tokyo). 2012;60(8):931-44. doi: |
| | 10.1248/cpb.c12-00258. PMID: 22863694. |
| Avermectin | Lili Dong, Jianjun Zhang, Research progress of avermectin: A minireview based on the structural |
| Derivative | derivatization of avermectin, Advanced Agrochem, Volume 1, Issue 2, 2022, Pages 100-112, ISSN 2773- |
| | 2371, https://doi.org/10.1016/j.aac.2022.11.001. |
| Avermectin | Fangfang Huang, Huan Ling, Jiao Li, Sen Lu, Weijie Liu, Qingshan Li, Fengbo Xu, Synthesis, bioactivities |
| Derivative | and 3D-QSAR of novel avermectin B2a aglycon derivatives, Chinese Chemical Letters, Volume 31, Issue 1, |
| | 2020, Pages 141-144, ISSN 1001-8417, https://doi.org/10.1016/j.cclet.2019.03.054. |
| Avermectin | Novel Avermectin derivative and preparation method thereof CN103421065A |
| Derivative | |
| Avermectin | Novel avermectin derivative, method for producing the same and microorganism producing the same. |
| Derivative | JP3100785B2 |
| | and many others |

wherein [HOST]ₙ is

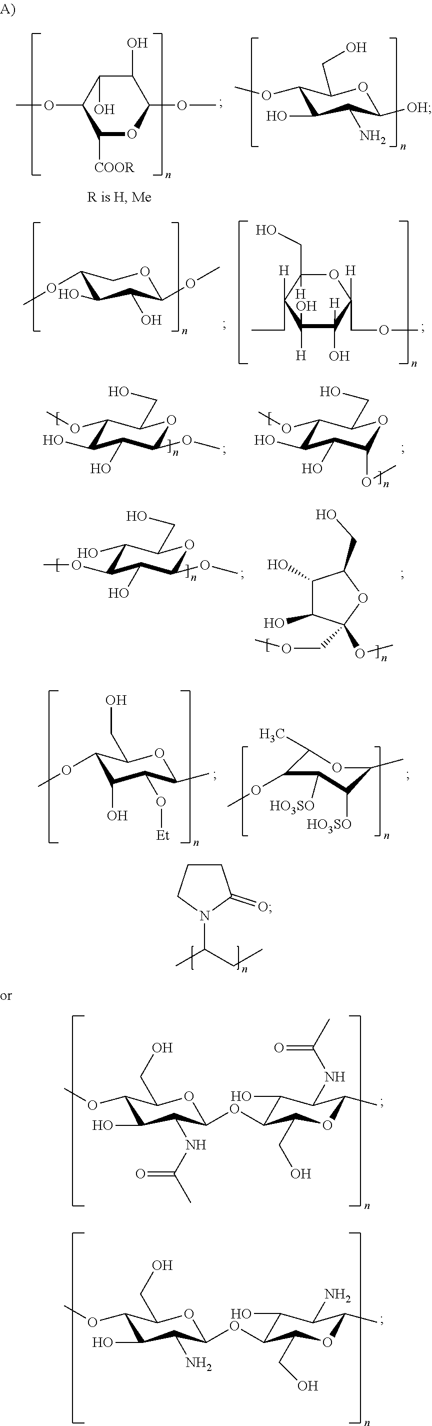

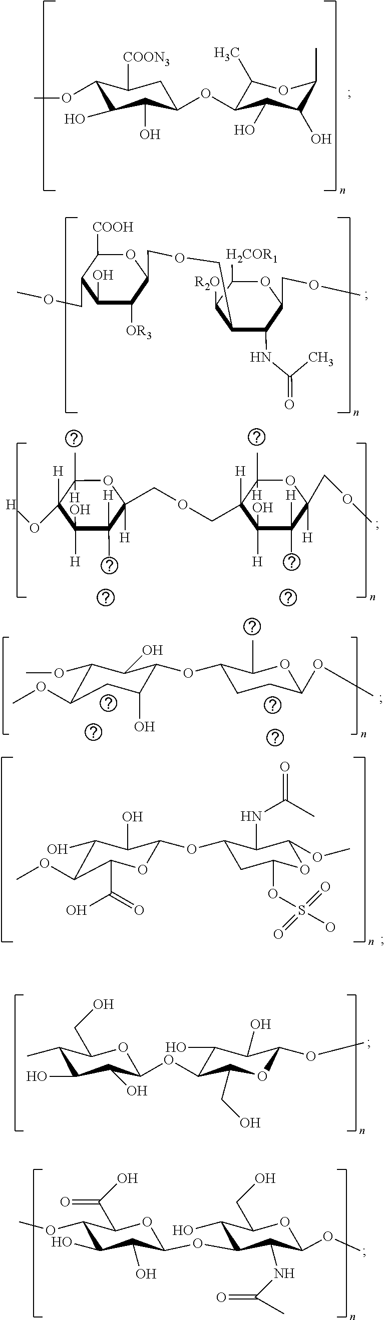

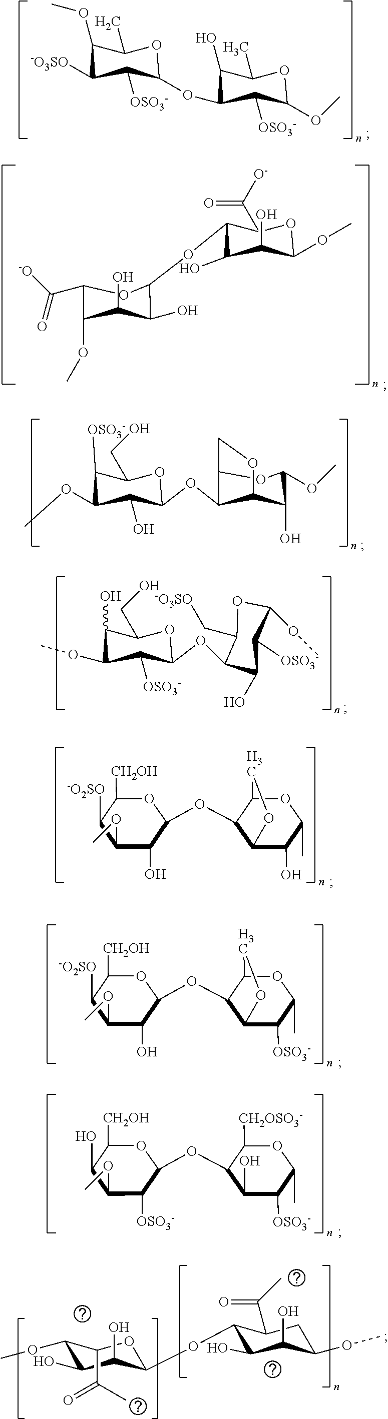

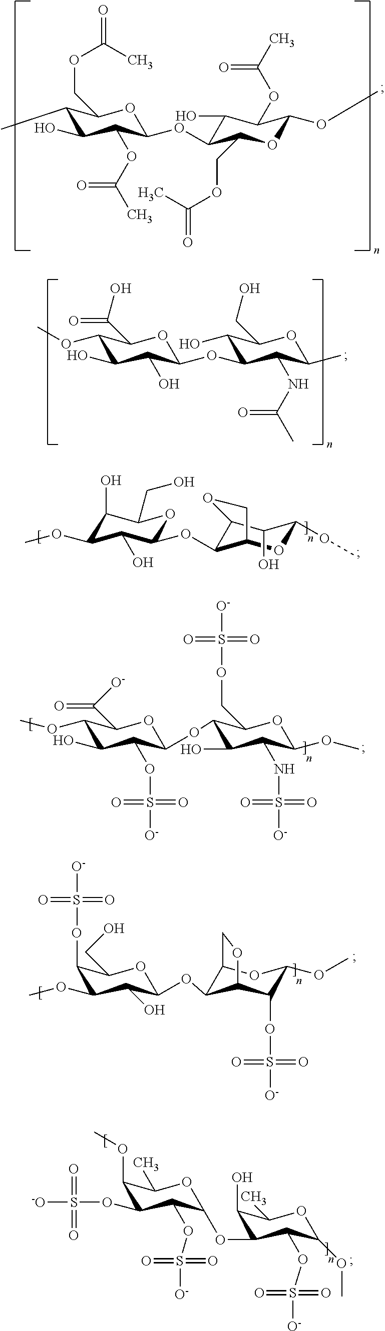

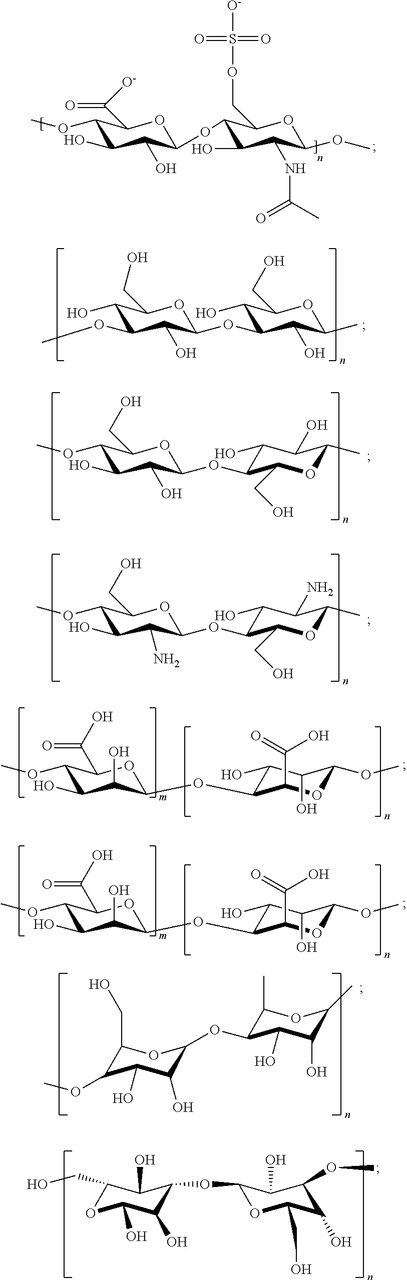

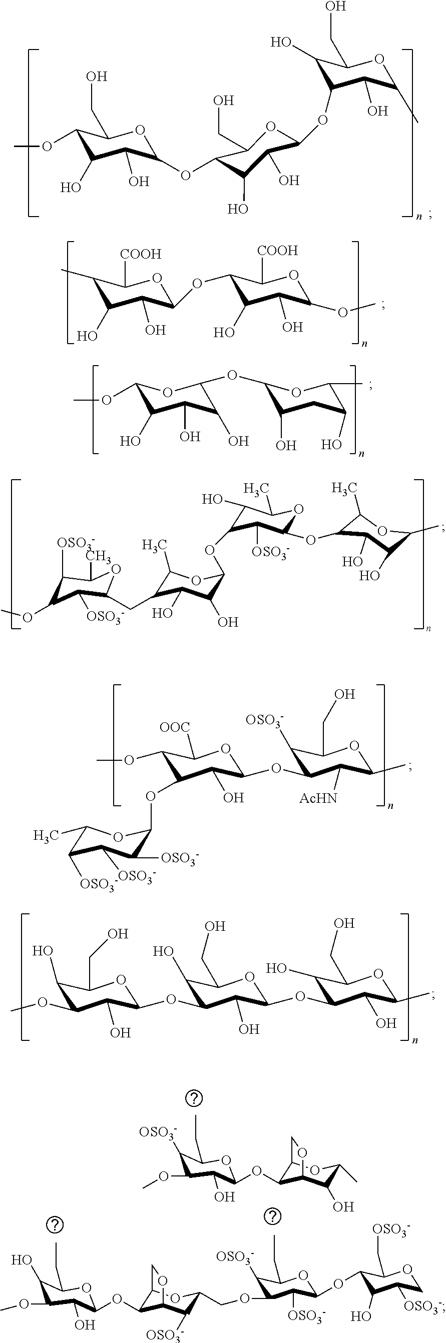

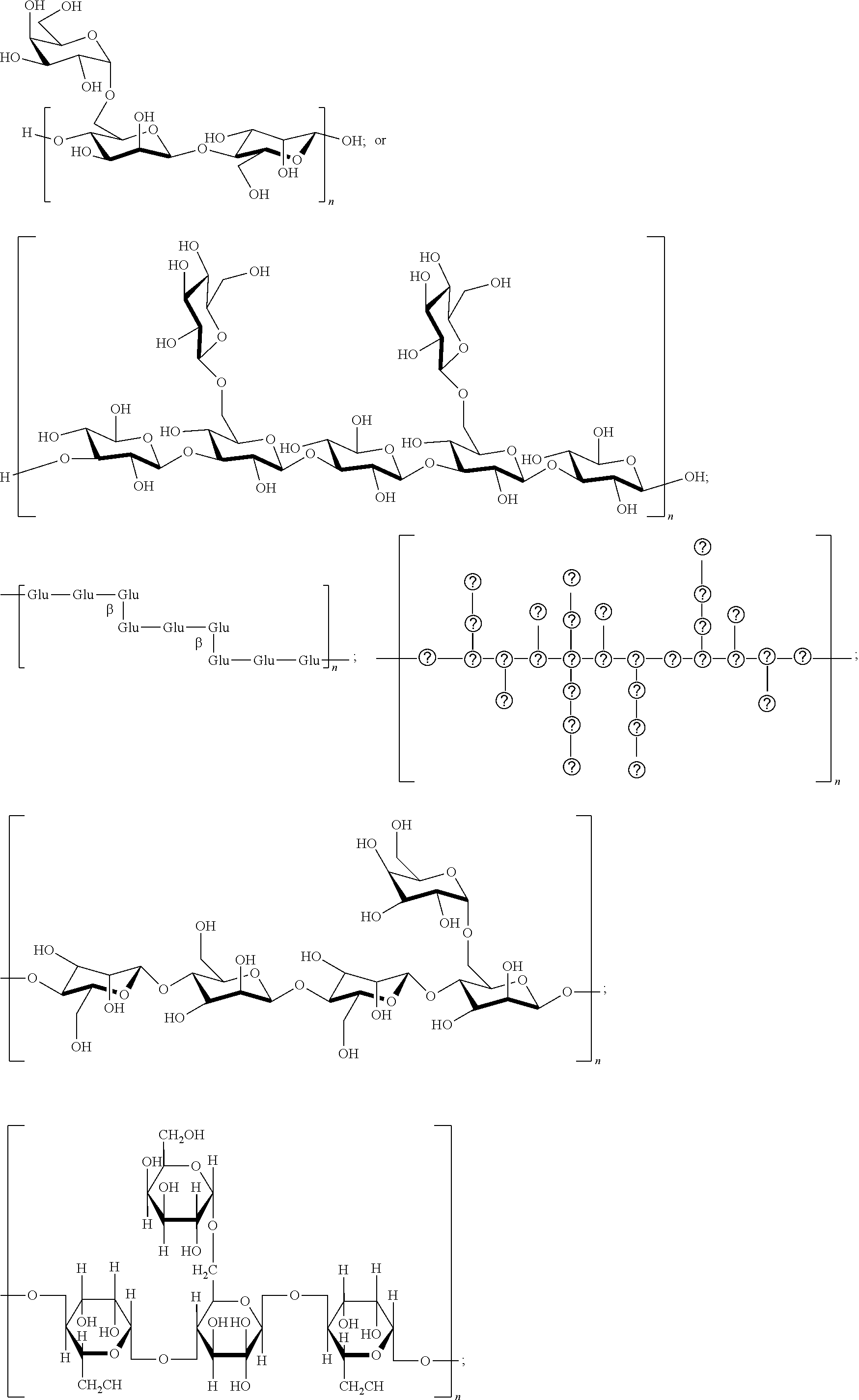

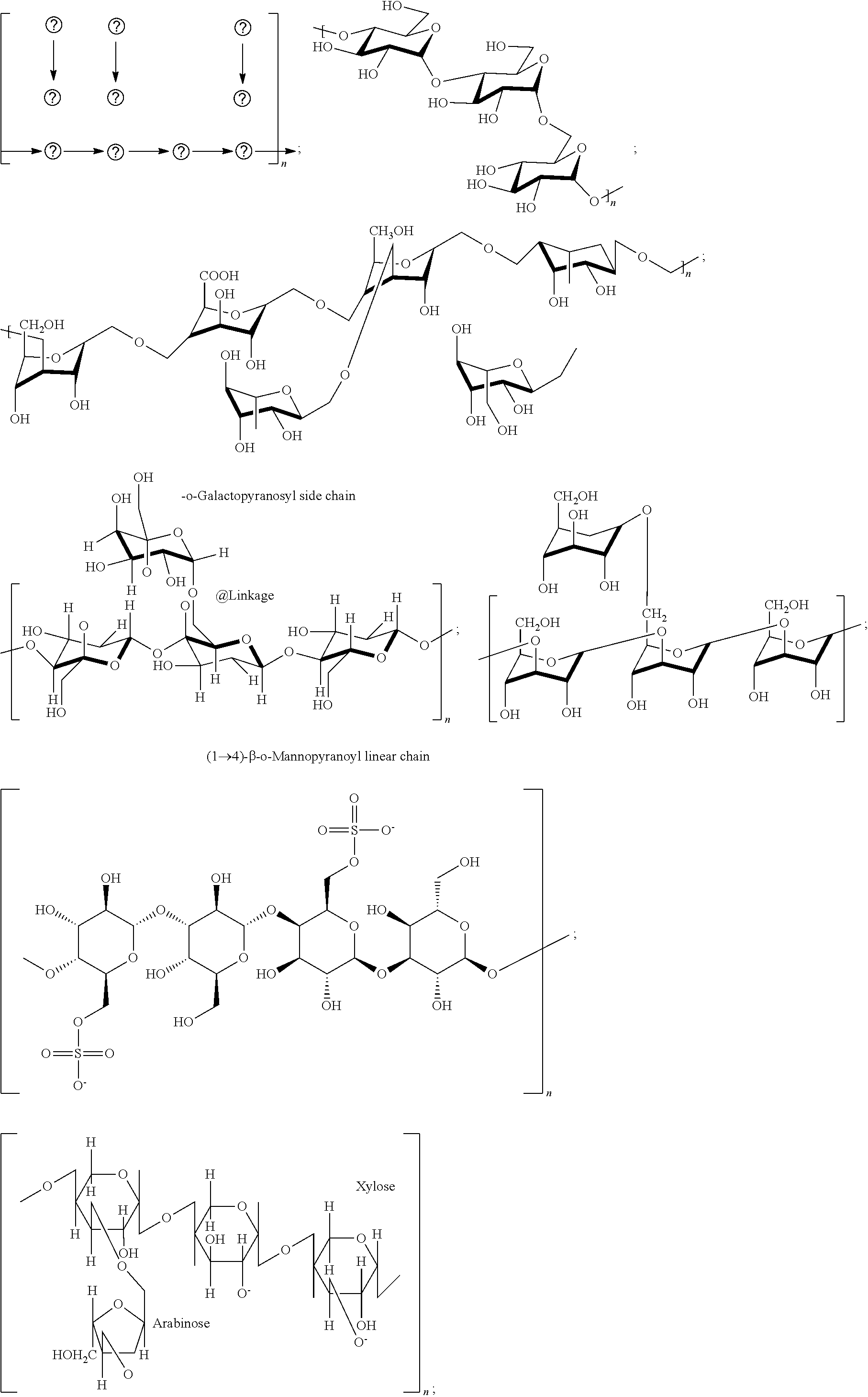

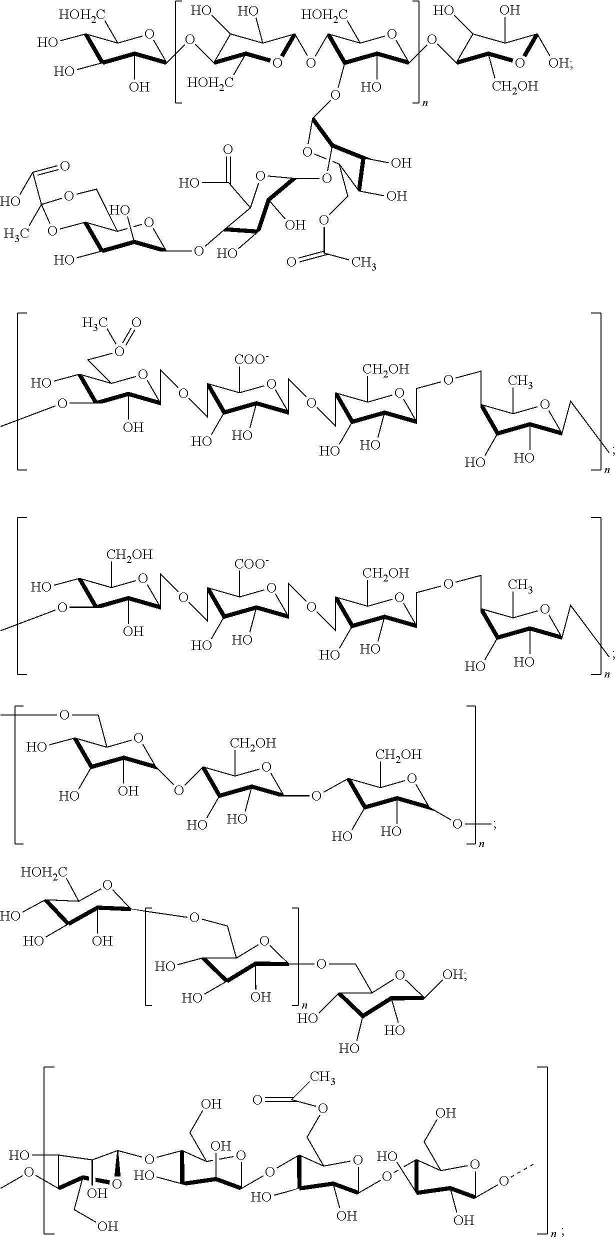

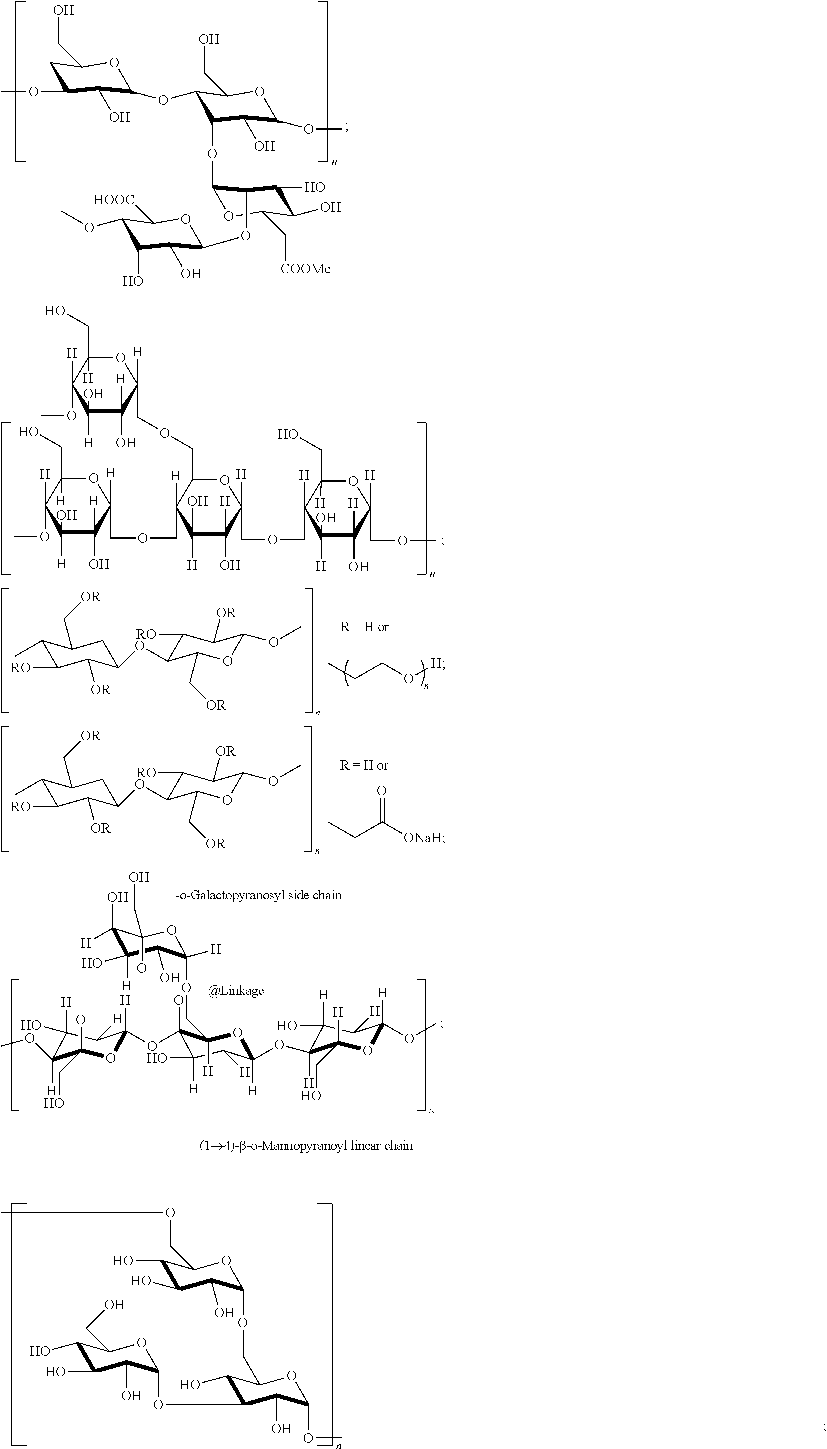

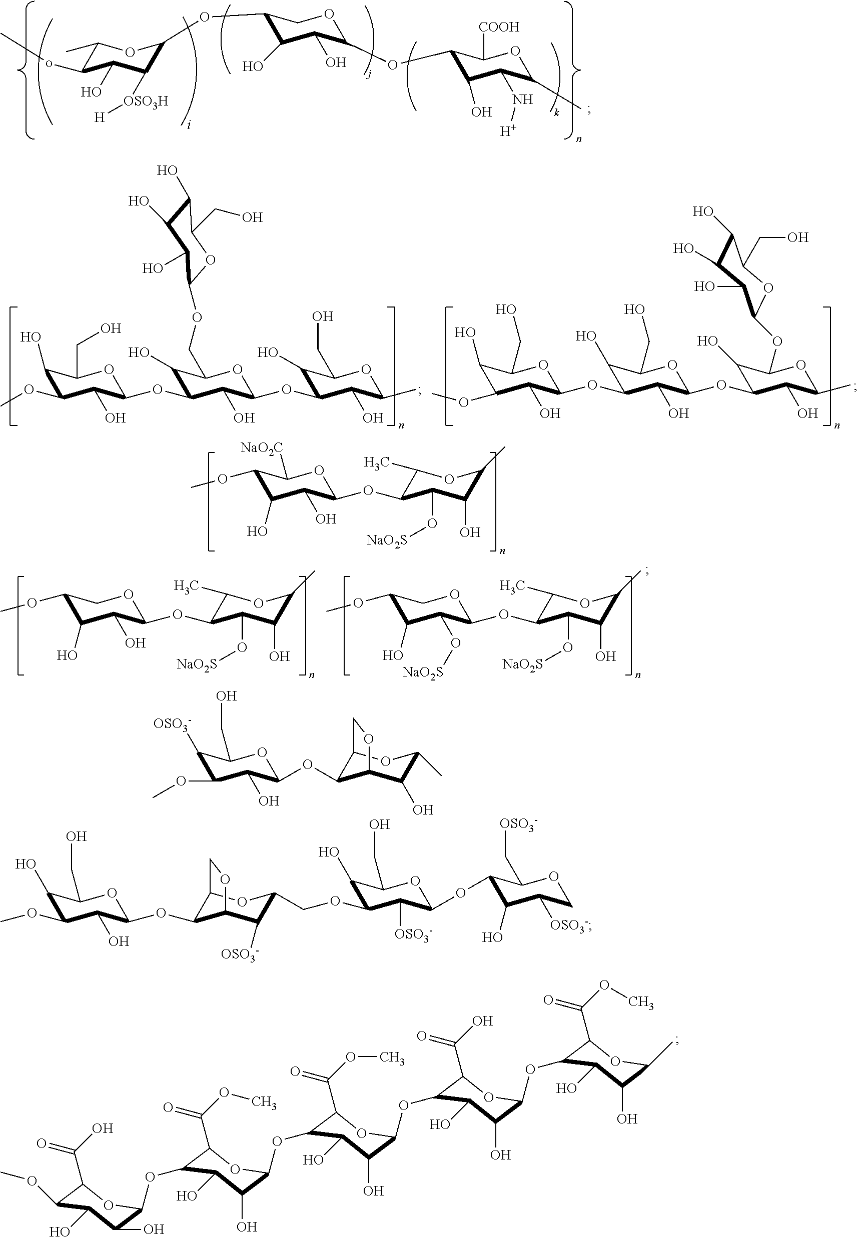

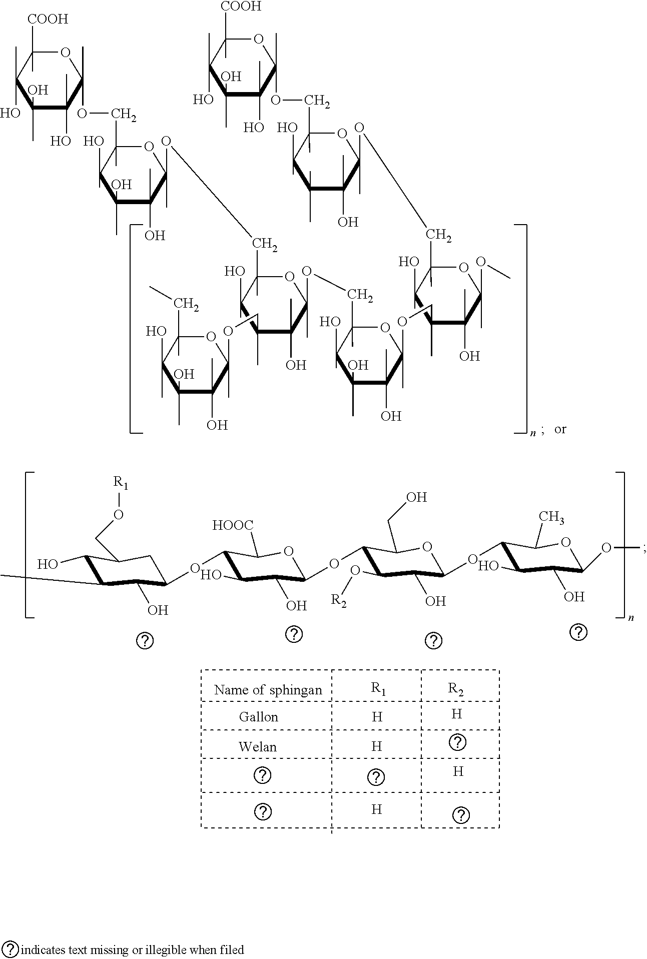

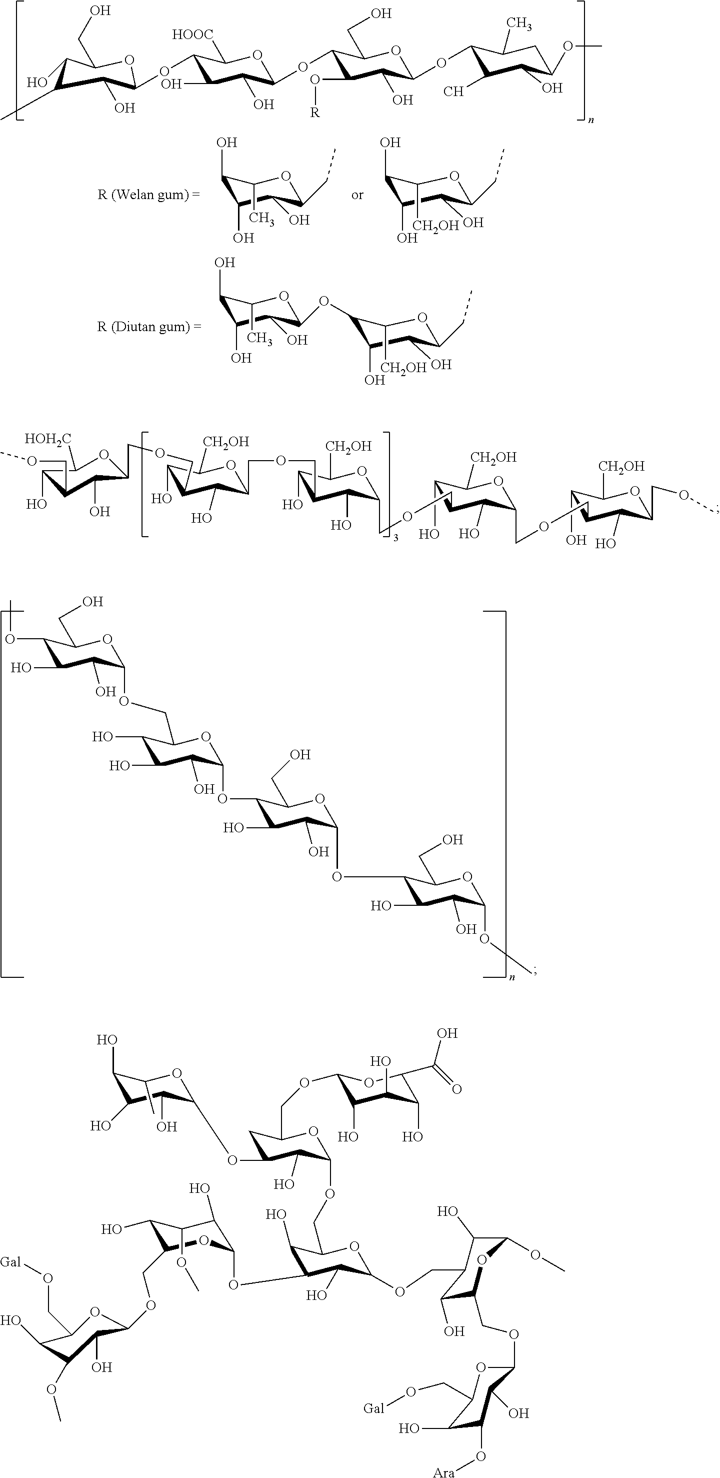

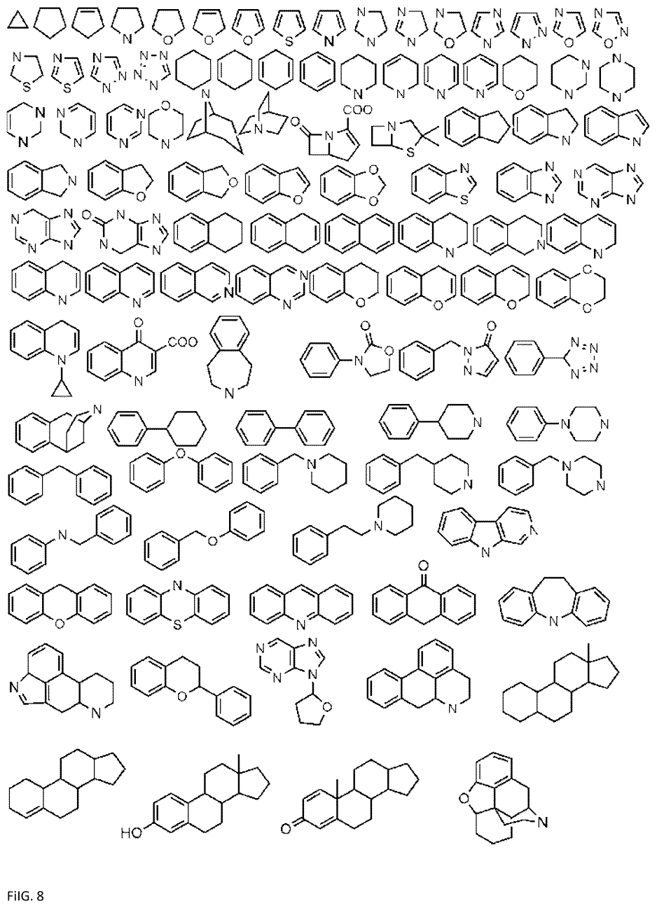

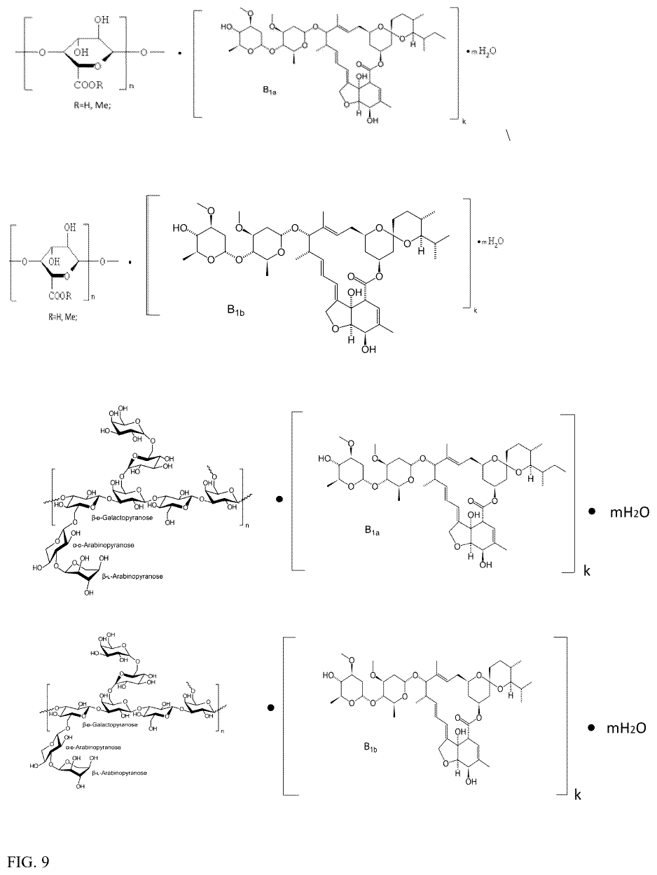

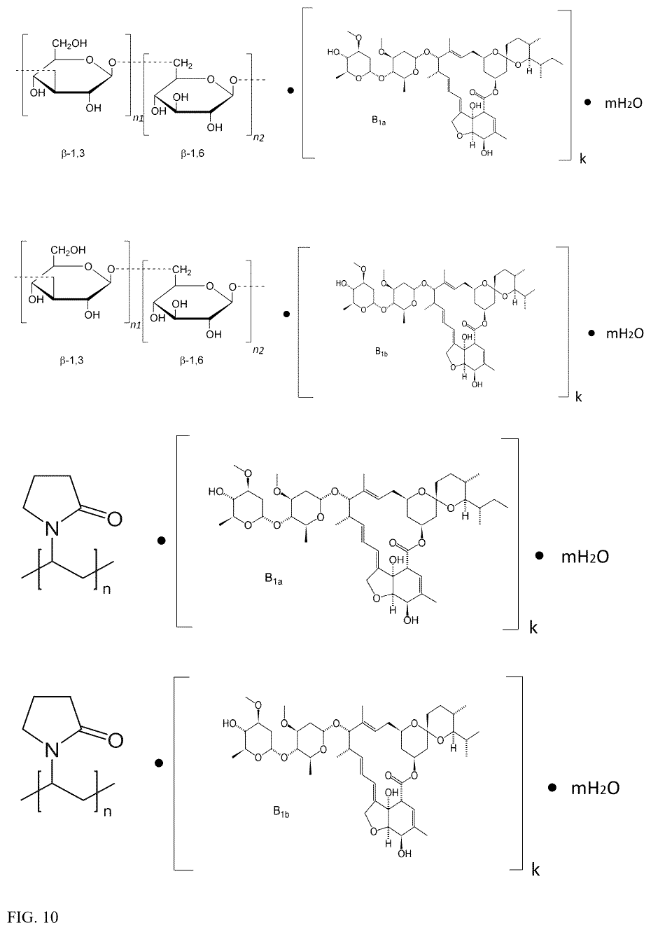

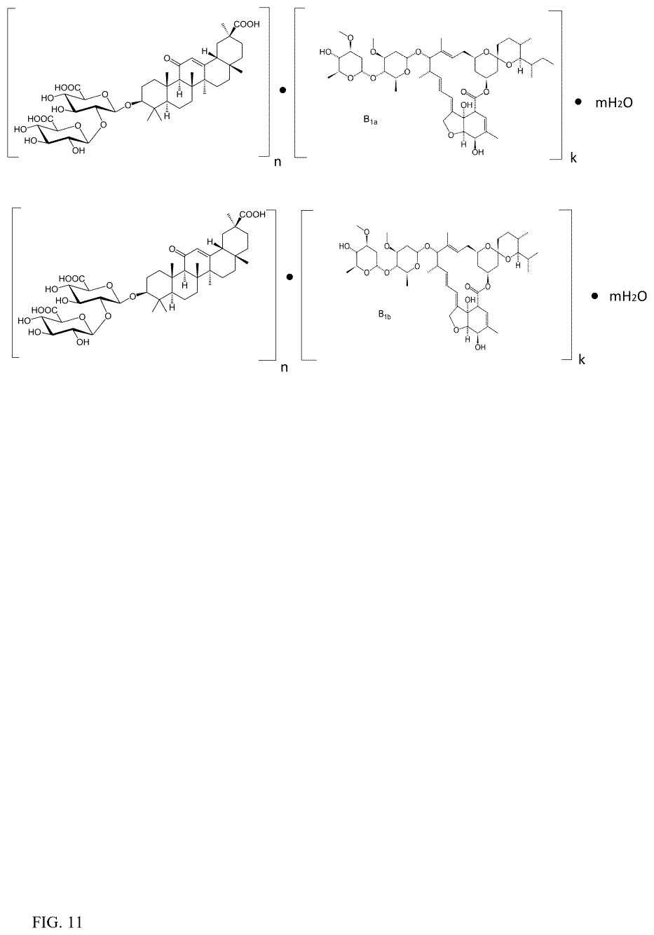

B) Chemically modified derivatives of (A) where any —OH can be replaced by R,
   wherein R is H, halogen, F, Cl, Br, I, OH, Me, Et, Ac, OMe, OEt, OAc, OR^{A}, N(R^{A})₂,N₃, CN, NO₂, S(O)₇R^{A}, (C₁-C₁₅)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, —C(═O)R^{A}, —C(═O)OR^{A}, —C(═O)NR^{A}R^{B}, —C(═O)SR^{A}, —S(O)R^{A}, —S(O)₂R^{A}, —S(O)(OR^{A}), —S(O)₂ (OR^{A}), —SO₂NR^{A}R^{B}, (C₆-C₂₀)aryl(C₁-C₈)alkyl, —PO(OH)₂, —PO(OH)OPO(OH)₂, —PO(OH)OPO(OH)O PO(OH)₂, —PO(OR^{A})₂, —POR^{A}R^{B}, phenyl, 1-naphthyl, 2-naphthyl, benzyl, (C₃-C₆)cycloalkyl, —CH₂—(C₃-C₆)cycloalkyl, —O—(C₁-C₈) alkyl, —O-benzyl, —O—CH₂—(C₃-C₆)cycloalkyl, CF₃, C(halogen)₃, —C(=Q)R^{A}, —C(=Q)OR^{A}, —C(=Q)N(R^{A}), —NR^{A}R^{B}, —⁺N(R^{A})₃, —SR^{A}, —S(O)R^{A}, —S(O)₂R^{A}, —S(O)(OR^{A}), —S(O)₂(OR^{A}), —OC(=Q)R^{A}, —OC(=Q)OR^{A}, —OC(=Q)(N(R^{A})₂), —SC(=Q)R^{A}, —SC(=Q)OR^{A}, —SC(=Q)(N(R^{A})₂), —N(R^{A})C(=Q)R^{A}, —N(R^{A})C(=Q)OR^{A}, —N(R^{A})C(=Q)N(R^{A})₂, —SO₂NR^{A}₂, —CN, —N₃, —NO₂, —OR^{A}, NR^{A}R^{B}, N(R^{A})OR^{B}, NR^{A}NR^{B}, N₃, NO, NO₂, CHO, CN, —CH(═NR^{A}), —CH═NNHR^{A}, —CH═N(OR^{A}), —CH(OR^{A})₂, —C(═O)NR^{B}R^{A}, —C(═S)NR^{B}R^{A}, —C(═O)OR^{A}, (C₆-C₂₀)optionally substituted aryl, optionally substituted heteroaryl, —C(═O)(C₁-C₈)alkyl, —S(O)ₙ(C₁-C₈)alkyl, SR^{A}, or selected from radicals obtained by removal of a hydrogen atom from the compounds shown in FIG. 8
      wherein Q is O, S, NR^{A}, ⁺N(O)(R^{A}), N(OR^{A}), ⁺N(O)(OR^{A}), or N—NR^{A}₂;
      wherein R^{A}, R^{B} is H, halogen, F, Cl, Br, I, OH, Et, Me, Ac, OR^{C}, N(R^{C})₂, N₃, CN, NO₂, S(O)_{z}R^{C}, (C₁-C₁₅)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, —C(═O)R^{C}, —C(═O)OR^{C}, —C(═O)NR^{C}R^{C}, —C(═O)SR^{C}, —S(O)R^{C}, —S(O)₂R^{C}, —S(O)(OR^{C}), —S(O)₂ (OR^{C}), SO₂NR^{C}R^{C}, (C₆-C₂₀)aryl(C₁-C₈)alkyl, —PO(OH)₂, —PO(OH)OPO(OH)₂, —PO(OH)OPO(OH)OPO(OH)₂, —PO(OR^{C})₂, —POR^{C}R^{C}, phenyl, 1-naphthyl, 2-naphthyl, benzyl, (C₃-C₆)cycloalkyl, —CH₂—(C₃-C₆)cycloalkyl, —O—(C₁-C₈) alkyl, —O-benzyl, —O—CH₂—(C₃-C₆)cycloalkyl, CF₃, C(halogen)₃, —C(O) R^{C}, —C(O)OR^{C}, —C(O)N(R^{C}), —NR^{C}R^{C}, —⁺N(R^{C})₃, —SR^{C}, —S(O) R^{C}, —S(O)₂R^{C}, —S(O)(OR^{C}), —S(O)₂(OR^{C}), —OC(O) R^{C}, —OC(O)OR^{C}, —OC(O)(N(R^{C})₂), —SC(=Q)R^{C}, —SC(=Q)OR^{C}, —SC(=Q)(N(R^{C})₂), —N(R^{C})C(=Q)R^{C}, —N(R^{C})C(=Q)OR^{C}, —N(R^{C})C(=Q)N(R^{C})₂, —SO₂NR^{C}₂, —CN, —N₃, —NO₂, —OR^{C}, NR^{C}R^{C}, N(R^{C})OR^{C}, NR^{C}NR^{C}, N₃, NO, NO₂, CHO, CN, —CH(═NR^{C}), —CH═NNHR^{C}, CH═N(OR^{C}), —CH(OR^{C})₂, —C(═O)NR^{C}R^{C}, —C(═S)NR^{C}R^{C}, —C(═O)OR^{C}, (C₆-C₂₀)optionally substituted aryl, optionally substituted heteroaryl, —C(═O)(C₁-C₈)alkyl, —S(O)z(C₁-C₈)alkyl, or SR^{C};
      wherein R^{C} is H, OH, F, Cl, Br, I, Me, Et, Ac, OMe, OEt, or OAc, or selected from radicals obtained by removal of a hydrogen atom from the compounds shown in FIG. 8;
      wherein z is from 1 to 10.
D) wherein [HOST]ₙ is
   D1) -[M1]ₙ-, -[M1ₓ₁-M2ₓ₂]ₙ-, -[M1ₓ₁-M2ₓ₂-M3ₓ₃]ₙ-, -[M1ₓ₁-M2ₓ₂-M3ₓ₃-M4ₓ₄]ₙ-, -[M1ₓ₁-M2ₓ₂-M3ₓ₃-M4ₓ₄-M5ₓ₅]ₙ-, -[M1ₓ₁-M2ₓ₂-M3ₓ₃-M4ₓ₄-M5ₓ₅-M6ₓ₆]ₙ-, [M1]ₙ-∫, [M1ₓ₁-M2ₓ₂]ₙ-, [M1ₓ₁-M2ₓ₂-M3ₓ₃]ₙ-∫, [M1ₓ₁-M2ₓ₂-M3ₓ₃-M4ₓ₄]ₙ-∫,[M1ₓ₁-M2ₓ₂-M3ₓ₃-M4ₓ₄-M5ₓ₅]ₙ∫, [M1ₓ₁-M2ₓ₂-M3ₓ₃-M4ₓ₄-M5ₓ₅-M6ₓ₆]ₙ-∫,

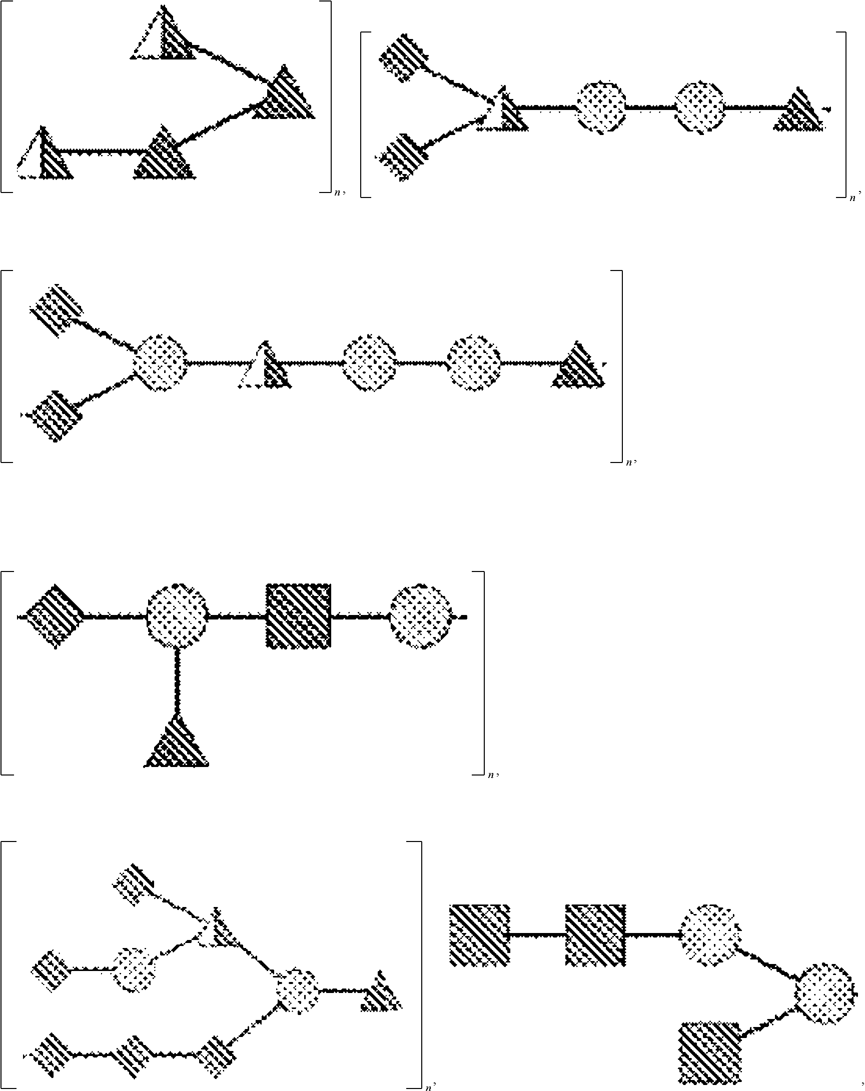

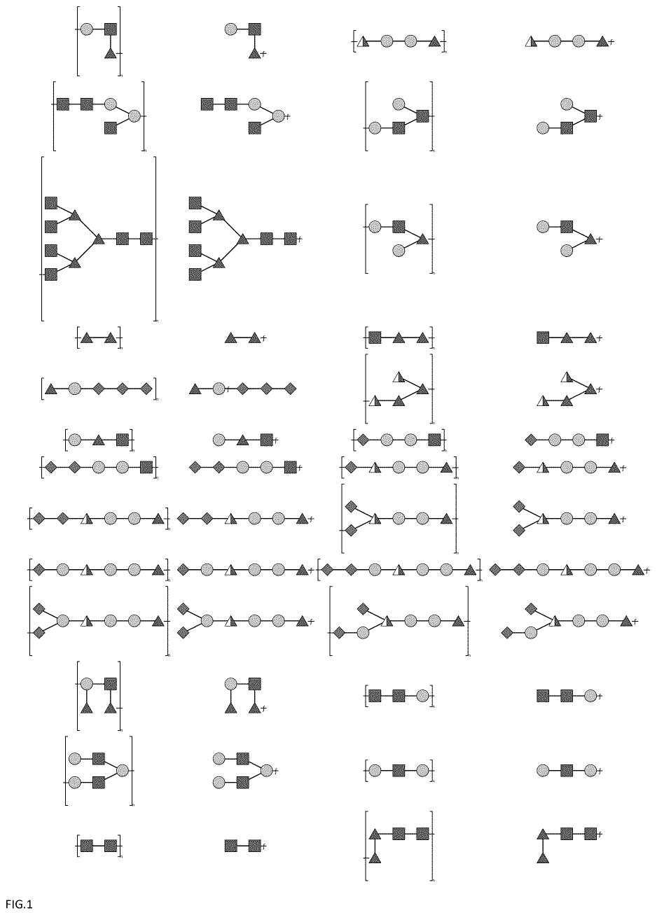

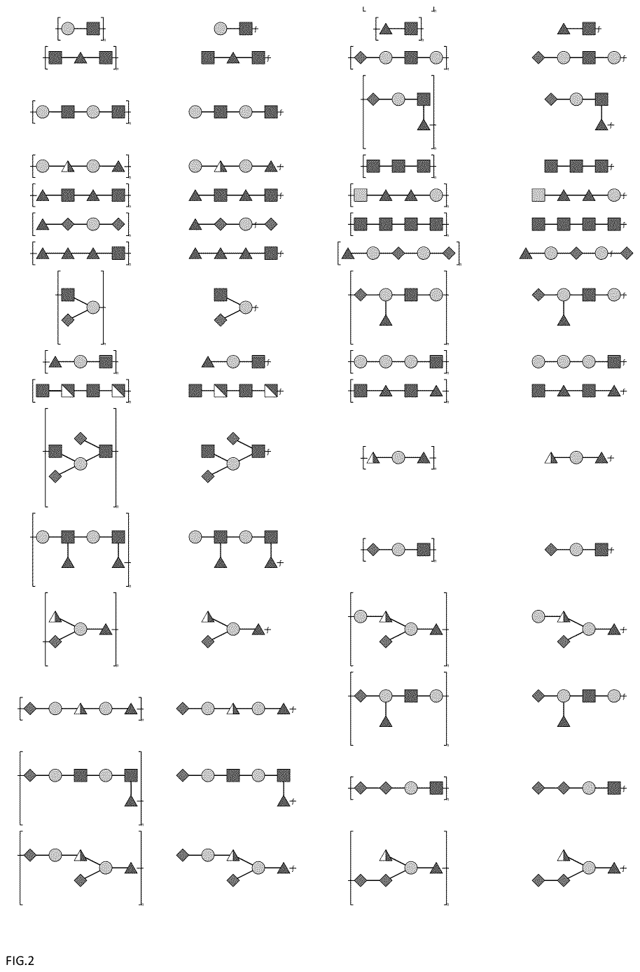

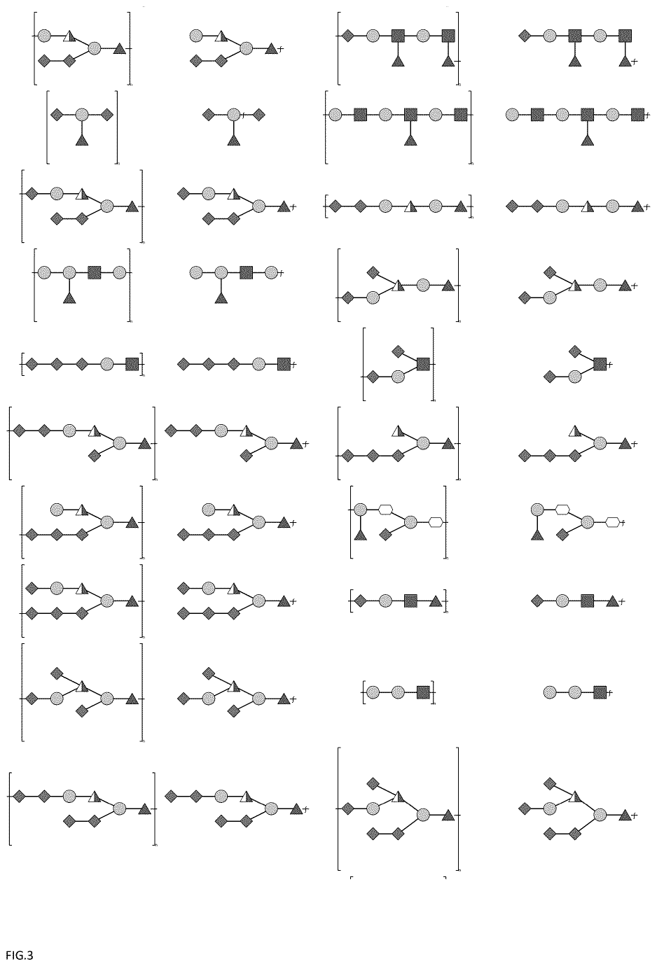

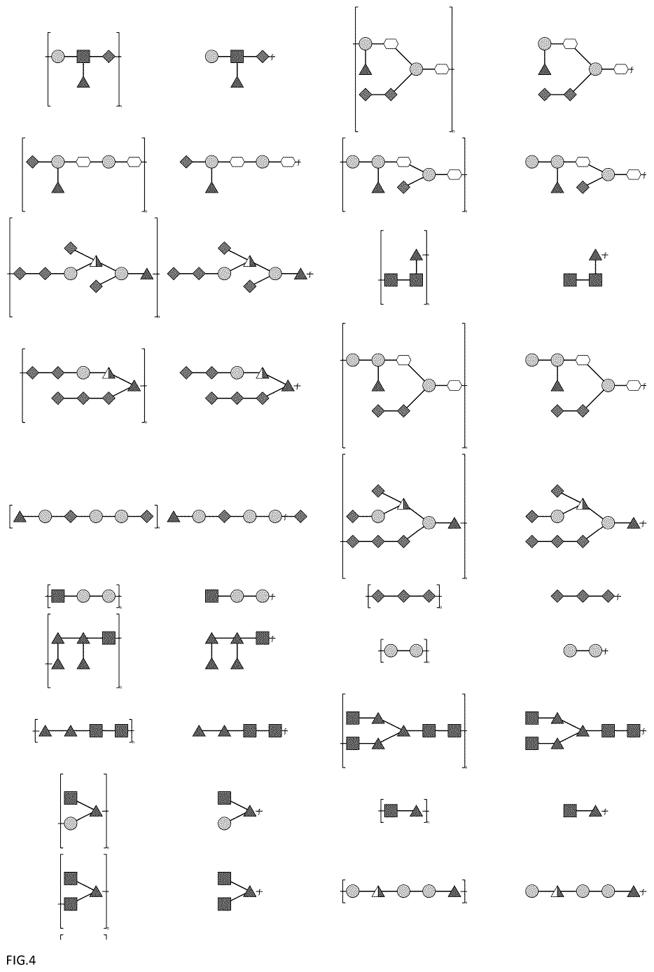

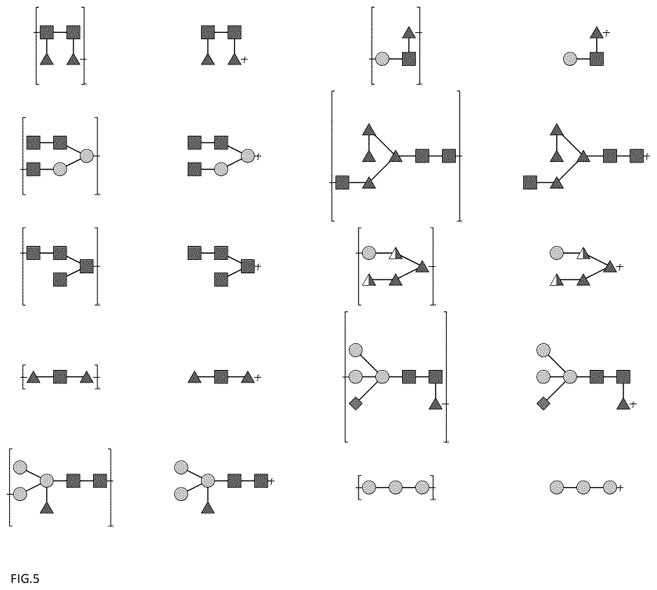

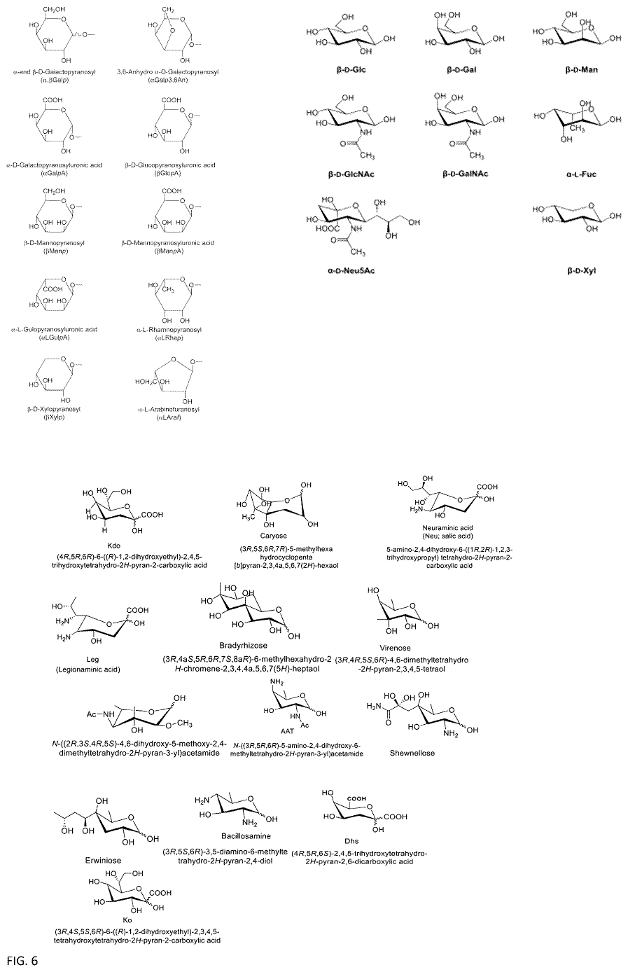

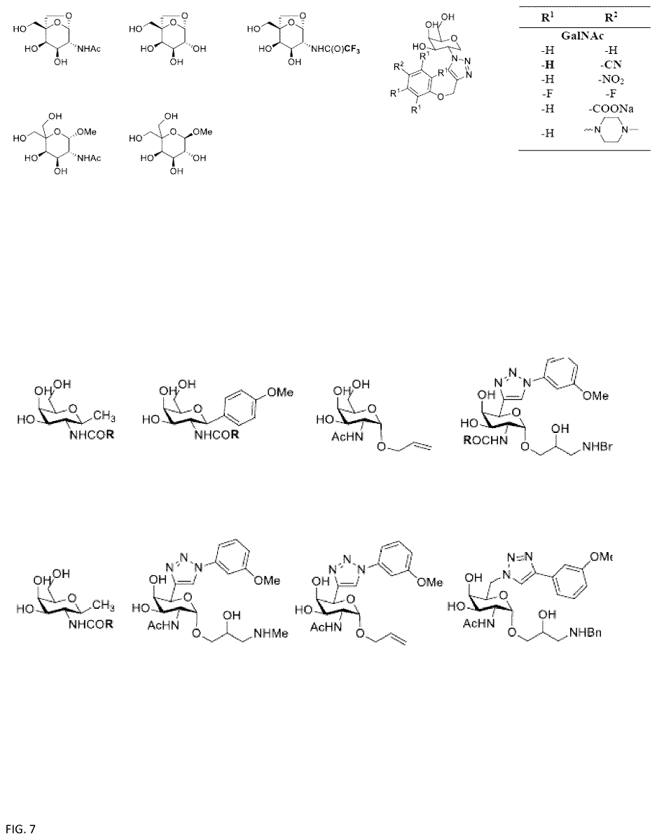

and continue on FIG. 1, FIG. 2, FIG. 3, FIG. 4, FIG. 5, and the like
   wherein x1, x2, x3, x4, x5, x6 is from 1 to 10⁸;
   wherein [symbol]
   is M1; [symbol] is M2; [symbol] is M3; [symbol]
   is M4; [symbol] is M5; [symbol] is M6;
   wherein at least one of M1, M2, M3, M3, M4, M5, M6 is a repeat unit(s) or terminal residue formed from this monomers:
   a) 4-epi-Legionaminic acid; 6-Deoxy-L-altrose; N-Acetyl-6-deoxy-L-altrosamine; 6-Deoxy-D-gulose; 6-Deoxy-D-talose; N-Acetyl-6-deoxy-D-talosamine; 8-epi-Acinetaminic acid; 8-epi-Legionaminic acid; Abequose; Acinetaminic acid; D-Allose; D-Alluronic acid; D-Allosamine; N-Acetyl-D-allosamine; L-Altrose; L-Altruronic acid; L-Altrosamine; N-Acetyl-L-altrosamine; L-Apiose; L-Arabinose; Bacillosamine; Colitose; D-glycero-D-manno-Heptose; 3-Deoxy-D-lyxo-heptulosaric acid; D-Digitoxose; D-Fructose; L-Fucose; N-Acetyl-L-fucosamine; D-Galactose; D-Galacturonic acid; D-Galactosamine; N-Acetyl-D-galactosamine; D-Glucose; D-Glucuronic acid; D-Glucosamine; N-Acetyl-D-glucosamine; D-Gulose; D-Guluronic acid; D-Gulosamine; N-Acetyl-D-gulosamine; L-Idose; L-Iduronic acid; L-Idosamine; N-Acetyl-L-idosamine; 2-Keto-3-deoxy-nononic acid; 3-Deoxy-D-manno-octulosonic acid; Legionaminic acid; L-glycero-D-manno-Heptose; D-Lyxose; D-Mannose; D-Mannuronic acid; D-Mannosamine; N-Acetyl-D-mannosamine; Muramic acid; N-Acetylmuramic acid; N-Glycolylmuramic acid; Neuraminic acid; N-Acetylneuraminic acid; N-Glycolylneuraminic acid; Olivose; Paratose; Pseudaminic acid; D-Psicose; D-Quinovose; N-Acetyl-D-quinovosamine; L-Rhamnose; N-Acetyl-L-rhamnosamine; D-Ribose; Sialic acid; L-Sorbose; D-Tagatose; D-Talose; D-Taluronic acid; D-Talosamine; N-Acetyl-D-talosamine; Tyvelose; D-XyloseDioses; Aldodiose; Glycolaldehyde; Trioses; Aldotriose; Glyceraldehyde; Ketotriose; Dihydroxyacetone; Tetroses; Aldotetroses; Erythrose Threose; Ketotetrose; Erythrulose; Pentoses; Aldopentoses; Arabinose; Lyxose; Ribose; Xylose; Ketopentoses; Ribulose; Xylulose; Deoxy sugars; Deoxyribose; Hexoses; Aldohexoses; Allose; Altrose; Galactose; Glucose; Gulose; Idose; Mannose; Talose; Ketohexoses; Fructose; Psicose; Sorbose; Tagatose; Fucose; Fuculose; Rhamnose; Heptoses; Ketoheptoses; Mannoheptulose; Sedoheptulose; Octoses; Nonoses; Neuraminic acid; galactosamine, N-acetylgalactosamine, N-formylgalactosamine, N-propionylgalactosamine, N-n-butanoylgalactosamine, N-iso-butanoylgalactosamine, lactose, lactobionic acid, mannose, aminoacids, nucleosides, folate, folic acid, 7-FBA-folate, and a-FBA-folate, and other folic acid chemical derivatives;
   and (using IUPAC nomenclature of organic chemistry, IUPAC Condensed code system) from this monomers:
   1,4-Anhydro-Gal; 1,4-Anhydro-Gal; 1,4-Anhydro-Kdo; 1dAlt-ol; 1dEry-ol; 2,3-Anhydro-All; 2,3-Anhydro-Man; 2,3-Anhydro-Rib; 2,5-Anhydro-D-Alt; 2,5-Anhydro-D-AltOS; 2,5-Anhydro-L-Man; 2,5-Anhydro-Man; 2,5-Anhydro-Man-ol; 2,5-Anhydro-ManOS; 2,5-Anhydro-Tal-ol; 2,5-Anhydro-TalOP; 2,7-Anhydro-Kdo; 2,7-Anhydro-Kdof; 3,6-Anhydro-Fruf; 3,6-Anhydro-Gal; 3,6-Anhydro-GalOS; 3,6-Anhydro-Glc; 3,6-Anhydro-L-Gal; 3,6-Anhydro-L-GalOMe; 3dLyxHepUlosaric; 4,7-Anhydro-KdoOPEtn; 4,8-Anhydro-DDGlcOct; 4,8-Anhydro-Kdo; 4,8-Anhydro-LDGlcOct; 4dAraHex; 4dEry-ol; 4eLeg5Ac7Ac; 6dAlt; 6dAltNAc; 6dAltOAc; 6dAltf; 6dAltfOAc; 6dGul; 6dManHep; 6dTal; 6dTalNAc; 6dTalNAcOAc; 6dTalOAc; 6dTalOAcOAc; 6dTalOAcOMe; 6dTalOMe; 6dTalOMe-ol; 6dTalf; 8eAciNAcNAc; 8eLeg; 8eLeg5Ac7Ac; 8eLeg5Ac7AcGro; 8eLegNAc; 8eLegNAcNBut; Abe; AbeOAc; AcefA; AciNAcNAc; Aco; AcoNAc; AllN; AllOAc; AllOMe; Alt; AltA; AltAN; AltNAcA; AltOMeA; Altf; AltfOAc; Ami; ApiOAc; ApiOMe-ol; Apif; Ara; Ara-ol; AraGlc; AraHepUloNAc-onic; AraHepUloNAcN-onic; AraHepUloNGc-onic; AraHexA; AraN; AraNMeOMe; AraOAc; AraOAcOP-ol; AraOMe; AraOPN; Araf; ArafGro; ArafOCoum; ArafOFer; ArafOMe; ArafOS; Asc; Bac; BacNAc; BoiOMe; Col; D-2dAraHex; D-2dAraHexA; D-3dAraHepUlosonic; D-3dLyxHepUlosaric; D-3dThrHexUlosonic; D-3dThrPen; D-3dXylHexOMe; D-4dAraHex; D-4dEryHexOAcN4en; D-4dLyxHex; D-4dLyxHexOMe; D-4dThrHexA4en; D-4dThrHexAN4en; D-4dThrHexOAcN4en; D-4dXylHex; D-6dAllOMe; D-6dAlt; D-6dAltHep; D-6dAltHepOMe; D-6dAltHepf; D-6dAraHex; D-6dAraHexN; D-6dAraHexNAc; D-6dAraHexOMe; D-6dLyxHexOMe; D-6dManHep; D-6dManHepOAc; D-6dManHepOP; D-6dTal; D-6dTalHep; D-6dTalOAc; D-6dTalOAcOMe; D-6dTalOMe; D-6dXylHex; D-6dXylHexN4Ulo; D-6dXylHexNAc4Ulo; D-6dXylHexOMe; D-7dLyxOctUlosonic; D-9dThrAltNon-onic; D-Alt; D-Apif; D-ApifOAc; D-ApifOMe; D-Ara; D-Ara-ol; D-AraHepUlo-onic; D-AraHex; D-AraHexUloOMe; D-AraN; D-AraOS; D-Araf; D-ArafN; D-Fuc; D-Fuc-ol; D-FucN; D-FucNAc; D-FucNAc-ol; D-FucNAcN; D-FucNAcNMe; D-FucNAcNMeN; D-FucNAcOAc; D-FucNAcOMe; D-FucNAcOP; D-FucNAcOPEtn; D-FucNAlaAc; D-FucNAsp; D-FucNBut; D-FucNButGro; D-FucNFo; D-FucNLac; D-FucNMeN; D-FucNN; D-FucNThrAc; D-FucOAc; D-FucOAcN; D-FucOAcNBut; D-FucOAcNGroA; D-FucOAcOBut; D-FucOAcOMe; D-FucOBut; D-FucOEtn; D-FucOMe; D-FucOMeN; D-FucOMeOCoum; D-FucOMeOFer; D-FucOMeOSin; D-FucOS; D-Fucf; D-FucfNAc; D-FucfOAc; D-Ido; D-IdoA; D-IdoOSA; D-Rha; D-Rha-ol; D-RhaCMe; D-RhaGro; D-RhaN; D-RhaNAc; D-RhaNAcOAc; D-RhaNBut; D-RhaNButOMe; D-RhaNFo; D-RhaOFoN; D-RhaOMe; D-RhaOMeN; D-RhaOP; D-RhaOS; D-RibHex; D-RibHexNAc; D-Sor; D-ThrHexA4en; D-ThrHexAN4en; D-ThrHexfNAc2en; D-ThrPen; D-Thre-ol; DDA1tHep; DDAltHepOMe; DDGalHep; DDGalHepOMe; DDGlcHep; DDManHep; DDManHepGroPA; DDManHepOBut; DDManHepOEtn; DDManHepOMe; DDManHepOP; DDManHepOPEtn; DDManNonUloNAcOFoN-onic; DLA1tNonUloNAc-onic; DLGalNonUloNAc-onic; DLGalNonUloNAcN; DLGalNonUloNAcN-onic; DLGlcHepOMe; DLHepGlcOMe; DLManHep; DLManHepOPEtn; Dha; Dig; DigCMe; DigOAc; DigOFo; DigOMe; Ery; Ery-L-GlcNonUloNAcOAcOMeSH-onic; Ery-ol; Ery-onic; EryHex; EryHex2en; EryHexA3en; EryOMe-onic; Fru; Fruf; FrufF; FrufI; FrufN; FrufNAc; FrufOAc; FrufOAcOBzOCoum; FrufOAcOFer; FrufOBzOCin; FrufOBzOCoum; FrufOBzOFer; FrufOFer; FrufOLau; Fuc; Fuc-ol; FucN; FucNAc; FucNAcA; FucNAcGroP; FucNAcN; FucNAcNMe; FucNAcOAc; FucNAcOMe; FucNAla; FucNAm; FucNBut; FucNFo; FucNProp; FucNThrAc; FucOAc; FucOAcNAm; FucOAcNBut; FucOAcOMe; FucOAcOSOMe; FucOMe; FucOMeOPam; FucOMeOVac; FucOP; FucOPOMe; FucOS; FucOSOMe; Fucf; Gal; Gal-ol; Gal6Ulo; GalA; GalA-ol; GalAAla; GalAAlaLys; GalAGroN; GalALys; GalAN; GalANCys; GalANCysAc; GalANSerAc; GalAOLac; GalAOPyr; GalASer; GalAThr; GalAThrAc; GalCl; GalF; GalGro; GalGroN; GalGroP; GaiN; GalNAc; GalNAc-ol; GalNAc-onic; GalNAcA; GalNAcAAla; GalNAcAN; GalNAcASer; GalNAcGro; GalNAcGroP; GalNAcGroPAN; GalNAcN; GalNAcOAc; GalNAcOAcA; GalNAcOAcAN; GalNAcOAcGroP; GalNAcOAcOMeA; GalNAcOAcOP; GalNAcOMe; GalNAcOP; GalNAcOPCho; GalNAcOPEtn; GalNAcOPyr; GalNAcOS; GalNAla; GalNAmA; GalNCysGly; GalNFoA; GaiNFoAN; GalNOPCho; GalNSuc; GalNonUloNAc-onic; GalOAc; GalOAcA; GalOAcAGroN; GalOAcAOLac; GalOAcAThr; GalOAcGroP; GalOAcN; GalOAcNAla; GalOAcNAmA; GalOAcNFoA; GalOAcNFoAN; GalOAcOFoA; GalOAcOMe; GalOAcOP; GalOAcOPyr; GalOFoAN; GalOFoNAN; GalOLac; GalOLac-ol; GalOMe; GalOMeA; GalOMeCl; GalOMeF; GalOMeNAla; GalOP; GalOPA; GalOPAEtn; GalOPAN; GalOPCho; GalOPEtn; GalOPEtnA; GalOPEtnN; GalOPy; GalOPyr; GalOS; GalOSA; GalOSOEt; GalOSOMeA; GalOctUloNAc-onic; Galf; GalfGro; GalfGroP; GalfNAc; GalfOAc; GalfOAcGro; GalfOAcGroP; GalfOAcOLac; GalfOLac; GalfOMe; GalfOP; GalfOPCho; GalfOPyr; Gl; Glc; Glc-ol; Glc6Ulo; GlcA; GlcAAla; GlcAAlaLys; GlcAGlu; GlcAGly; GlcAGro; GlcAGroN; GlcALys; GlcAN; GlcAOLac; GlcAOPy; GlcAOPyr; GlcASer; GlcAThr; GlcAThrAc; GlcCho; GlcF; GlcGro; GlcGroA; GlcGroP; GlcGroPA; GlcI; GlcN; GlcN-ol; GlcNAc; GlcNAc-ol; GlcNAcA; GlcNAcAAla; GlcNAcAN; GlcNAcANAla; GlcNAcANAlaAc; GlcNAcANAlaFo; GlcNAcAla; GlcNAcCl; GlcNAcGlu; GlcNAcGly; GlcNAcGro; GlcNAcGroP; GlcNAcGroPA; GlcNAcI; GlcNAcN; GlcNAcN-ol; GlcNAcNAla; GlcNAcNAlaFo; GlcNAcNAmA; GlcNAcNButA; GlcNAcOAc; GlcNAcOAcA; GlcNAcOAcN; GlcNAcOAcNAla; GlcNAcOAcOCmOOle; GlcNAcOAcOCmOPam; GlcNAcOAcOCmOVac; GlcNAcOAcOLac; GlcNAcOAcOOle; GlcNAcOAcOPam; GlcNAcOAcOPyr; GlcNAcOAcOS-ol; GlcNAcOAcOVac; GlcNAcOGc; GlcNAcOLac; GlcNAcOLacAla; GlcNAcOLacGro; GlcNAcOMe; GlcNAcOMeA; GlcNAcOP; GlcNAcOPCho; GlcNAcOPEtg; GlcNAcOPEtn; GlcNAcOPOAch; GlcNAcOPyr; GlcNAcOS; GlcNAcOS-ol; GlcNAcOSA; GlcNAm; GlcNAmA; GlcNBut; GlcNButAN; GlcNButOAc; GlcNCmOCm; GlcNCmOCmOOle; GlcNCmOCmOVac; GlcNCmOVac; GlcNGc; GlcNGly; GlcNMe; GlcNMeOCm; GlcNMeOCmOPam; GlcNMeOCmOSte; GlcNMeOCmOVac; GlcNMeOSte; GlcNMeOVac; GlcNN; GlcNOAep; GlcNOCmOAch; GlcNOCmOVac; GlcNOMar; GlcNOMe; GlcNOMyr; GlcNOOle; GlcNOPam; GlcNOPyr; GlcNOSte; GlcNOVac; GlcNS; GlcNSOS; GlcNSOSOMe; GlcNSuc; GlcOAc; GlcOAcA; GlcOAcGro; GlcOAcGroA; GlcOAcGroP; GlcOAcN; GlcOAcNBut; GlcOAcNCmOOle; GlcOAcNCmOPam; GlcOAcNCmOVac; GlcOAcNMeOCm; GlcOAcNMeOCmOVac; GlcOAcNMeOVac; GlcOAcNOCmOVac; GlcOAcNOOle; GlcOAcNOPam; GlcOAcNOVac; GlcOAcOCoum; GlcOAcOFer; GlcOAcOOle; GlcOAcOP; GlcOAcOPam; GlcOAcOS; GlcOAcOSA; GlcOAcOSte; GlcOButA; GlcOBz; GlcOCoum; GlcOEt; GlcOEtn; GlcOEtnA; GlcOEtnN; GlcOFer; GlcOFoN; GlcOGc; GlcOLac; GlcOMal; GlcOMe; GlcOMe-ol; GlcOMeA; GlcOMeAN; GlcOMeN; GlcOMeNOMyr; GlcOMeOFoA; GlcOMeOPyr; GlcOOle; GlcOP; GlcOP-ol; GlcOPA; GlcOPCho; GlcOPChoGro; GlcOPEtn; GlcOPEtnGro; GlcOPEtnN; GlcOPGroP; GlcOPN; GlcOPNOMyr; GlcOPNOPam; GlcOPOOle; GlcOPPEtn; GlcOPPEtnN; GlcOPam; GlcOPyr; GlcOS; GlcOSA; GlcOSN; GlcOSNMeOCm; GlcOSOEt; GlcOSOMe; GlcOSOMeA; GlcOSin; GlcS; GlcSH; GlcThr; Glef; Gro; Gro-ol; Gul; GulAN; GulNAcA; GulNAcAN; GulNAcNAmA; GulNAcOAcA; Hep; HepOP; HepOPEtn; HepOPPEtn; Hex; HexA; HexN; HexNAc; HexOMeOFo; Hexf; Ido; IdoA; IdoN; IdoNAc; IdoOAcA; IdoOAcOSA; IdoOMeA; IdoOS; IdoOSA; IdoOSOEtA; IdoOSOMeA; Kdn; KdnOAc; KdnOMe; KdnOPyr; Kdo; Kdo-ol; KdoGroP; KdoN; KdoOAc; KdoOAcOS; KdoOMe; KdoOP; KdoOPEtn; KdoOPN; KdoOPOEtn; KdoOPOPEtn; KdoOPPEtn; KdoOPPEtnN; KdoOPyr; KdoOS; Kdof; Ko; KoOMe; KoOPEtn; L-4dEryHexAN4en; L-4dThrHex4en; L-4dThrHexA4en; L-4dThrHexA4enAla; L-4dThrHexAN4en; L-4dThre-ol; L-6dAraHex; L-6dAraHexOMe; L-6dGalHep; L-6dGalHepOP; L-6dGulHep; L-6dGulHepOMe; L-6dGulHepOP; L-6dXylHexNAc4Ulo; L-Aco; L-AcoOMe; L-AcoOMeOFo; L-BoiOMe; L-Cym; L-CymOAc; L-DigOMe; L-Ery; L-EryCMeOH; L-EryHexA4en; L-Fru; L-Fruf; L-Gal; L-GalAN; L-GalNAc; L-GalNAc-onic; L-GalNAcA; L-GalNAcAN; L-GalNAcOAcA; L-GalNAmA; L-GalOAcNAmA; L-GalOS; L-Glc; L-GlcA; L-GlcNAc; L-GlcOMe; L-Gro-onic; L-GroHexUlo; L-Gul; L-Gul-onic; L-GulA; L-GulAN; L-GulHep; L-Gu1NAc; L-Gu1NAcA; L-Gu1NAcAGly; L-GulNAcAN; L-Gu1NAcANEtn; L-Gu1NAcNAmA; L-Gu1NAcNEtnA; L-GulNAcOAc; L-Gu1NAcOAcA; L-Gu1NAcOAcAN; L-Gu1NAcOEtA; L-GulNAcOEtnA; L-GulOAcA; L-Lyx; L-LyxHex; L-LyxHexNMe; L-LyxHexOMe; L-Man; L-ManOMe; L-ManOctUlo-onic; L-Ole; L-OleOAc; L-Oli; L-OliOMe; L-Qui; L-QuiN; L-QuiNAc; L-QuiNAcOMe; L-QuiNAcOP; L-QuiOMeN; L-RibHex; L-Ribf; L-Tal; L-The; L-TheOAc; L-Thr; L-ThrHexA4en; L-ThrHexAN4en; L-ThrHexOMe4en; L-ThrHexOMeA4en; L-ThrHexOSA4en; L-Xyl; L-XylHex; L-XylOMe; LDGa1Hep; LDGalNonUloNAc-onic; LDGlcHep; LDIdoHep; LDIdoHepPro; LDManHep; LDManHepGroN; LDManHepGroPA; LDManHepOAc; LDManHepOCm; LDManHepOEtn; LDManHepOMe; LDManHepOP; LDManHepOPEtn; LDManHepOPEtnOEtn; LDManHepOPOCm; LDManHepOPOMe; LDManHepOPOPEtn; LDManHepOPOPPEtn; LDManHepOPPEtn; LDManHepOPPEtnOPyrP; LDManHepOPyrP; LDManNonUloNAcOFoN-onic; LDManNonUloOFoNN-onic; LLManNonUloOFoN-onic; Leg; Leg5Ac7Ac; LegNAc; LegNAcAla; LegNAcNAla; LegNAcNAm; LegNAcNBut; LegNFo; Lyx; LyxHex; LyxHexOMe; LyxOMe; LyxOctUlo-onic; Lyxf; Man; Man-ol; ManA; ManCMe; ManF; ManGroP; ManN; ManNAc; ManNAcA; ManNAcAAla; ManNAcAGro; ManNAcAN; ManNAcANOOrn; ManNAcASer; ManNAcAThr; ManNAcGroA; ManNAcGroP; ManNAcGroPA; ManNAcNAmA; ManNAcNEtnA; ManNAcOAc; ManNAcOAcA; ManNAcOLac; ManNAcOMe; ManNAcOMeAN; ManNAcOPEtn; ManNAcOPyr; ManNBut; ManNGroP; ManNonUloNAc-onic; ManOAc; ManOAcA; ManOAcN; ManOAcOMe; ManOAcOPyr; ManOAep; ManOBut; ManOEtn; ManOLac; ManOMe; ManOMeA; ManOP; ManOP-ol; ManOPCho; ManOPEtn; ManOPOMe; ManOPOPyr-ol; ManOPy; ManOPyr; ManOS; ManOctUlo; ManSH; Manf; Mur; MurNAc; MurNAcAla; MurNAcOP; MurNAcSer; Neu; Neu5Ac; Neu5AcN; Neu5AcNAc; Neu5AcNMe; Neu5AcOAc; Neu5AcOAcOMe; Neu5AcOGc; Neu5AcOMe; Neu5AcOS; Neu5Gc; Neu5GcA; Neu5GcN; Neu5GcOMe; Neu5GcOS; NeuOFo; NeuOMe; OLac; Ole; Oli; OliN; OliNAc; OliOMe; Par; Parf; PerNAc; Pse; Pse5Ac7Ac; Pse5Ac7AcNBut; Pse5Ac7AcOBut; PseNAc; PseNAcNAm; PseNAcNBut; PseNAcNFo; PseNAcNGro; PseNAcOAcNBut; PseNAcOBut; PseNButNFo; PseNGcNAm; PseOAc; PseOAcOFo; PseOFo; Qui; QuiN; QuiNAc; QuiNAc-ol; QuiNAcGro; QuiNAcGroP; QuiNAcN; QuiNAcNAlaAc; QuiNAcNAm; QuiNAcNAspAc; QuiNAcNBut; QuiNAcNButGro; QuiNAcNGroA; QuiNAcOAc; QuiNAcOBut; QuiNAcOMe; QuiNAcOP; QuiNAla; QuiNAlaAc; QuiNAlaAcGro; QuiNAlaBut; QuiNAlaButGro; QuiNAspAc; QuiNBut; QuiNButAla; QuiNButOMe; QuiNFo; QuiNGlyAc; QuiNHse; QuiNHseGro; QuiNLac; QuiNMal; QuiNSerAc; QuiNThrAc; QuiOMe; QuiOMeN; QuiOS; QuiOSN; QuiOSNBut; Rha; Rha-ol; RhaCMe; RhaCl; RhaGro; RhaGroA; RhaGroP; RhaNAc; RhaNAcNBut; RhaNAcNFo; RhaNAcOAc; RhaNPro; RhaOAc; RhaOAcOLac; RhaOAcOMe; RhaOBut; RhaOFer; RhaOLac; RhaOMe; RhaOMeCMeNLac; RhaOMeCMeOFo; RhaOP; RhaOPEtn; RhaOPOMe; RhaOProp; RhaOPyr; RhaOS; Rhaf; Rib; Rib-ol; RibGroP-ol; RibOAc; RibOAcOP-ol; RibOP-ol; RibOPEtn-ol; RibOPGroP-ol; Ribf; Ribf-uronic; RibfOAc; Sed; Sedf; Sor; Sorf; Sue; Sug; SugOAc; Tag; Tal; The; Thr; Thre-ol; Thre-onic; Tyv; VioNAc; Xluf; XlufOMe; Xyl; Xyl-ol; Xyl-onic; XylHex; XylHexNAc; XylHexUlo; XylHexUloN; XylHexUloNAc; XylNAc; XylNMe; XylOAc; XylOBz; XylOMe; XylOP; XylOS; Xylf; Yer; YerOAc; a-Tri-ol; a-Tri-onic; aldehyde-2,5-Anhydro-L-Man; aldehyde-2,5-Anhydro-Tal; aldehyde-Gro; aldehyde-Hex; aldehyde-L-Gro; aldehyde-L-GroN; aldehyde-QuiNAc; aldehyde-Rib; aldehyde-a-Tri-ol; aldehyde-b-Tri-ol; b-Tri-N-ol; b-Tri-OP-ol; b-Tri-ol; b-Tri-onic; b1-4Glc; cNeu5Ac;
   b) and from monomers listed in FIG. 6,7 and the like, and chemical derivatives thereof;
   c) deoxysugars of (a), (b) (Deoxyribose, Rhamnose, Fuculose, Fucose, and the like);
   d) uronic acids of (a), (b), (c)—(glucuronic acid, iduronic acid, etc), which may be methoxylated, ethoxylated or acetylated;
   e) (a), (b), (c), (d) in which a hydroxyl group has been replaced with an amine group—amino sugars (galactosamine, glucosamine, sialic acid, N-acetylglucosamine, N-acetylgalactosamine, N-acetylneuraminic acid and the like (more then 60 aminosugars));
   f) sulfonic acid derivatives of (a), (b), (c), (d), (e)—sulfosugars (O-, N-sulfated, and the like);
   g) acetyl; Anhydro, D-alanyl; N-acetyl-D-alanyl; N-acetimidoyl; N—(N-methyl-acetimidoyl); N—(N,N-dimethyl-acetimidoyl); formyl; glycolyl; N-acetyl-glutaminyl; N-methyl-5-glutamyl; glycyl; glyceryl; 2,3-di-O-methyl-glyceryl; 4-hydroxybutyryl; 3,4-dihydroxybutyryl; (R)-3-hydroxybutyryl; (S)-3-hydroxybutyryl; lactyl; methyl; amino; N-acetyl; phosphate; pyruvyl; 1-carboxyethylidene; sulfate; tauryl; Me; Et; Pr; Bu; Pe; Hx; Hp; Oc; Nn; Dec; Und; Dod; Acyl; RCO—; Fo (or HCO); Ac; Gc; Pp; Br (Butyryl); V1 (valeryl); Hxo; Hpo; Oco; Nno; Dco; Udo; Lau; Myr; Pam; Ste; eSte; Lin; D4Ach; Gro; Gra; Grn; Gri; Ose; Gled; Gal; Fuc; GlcA; GlcUe; GlcN; GlcNAc; Neu; Sia; Mur; NeuAcg; NeuGc; Cer; Cho; Etnh; Ins; Ser; Ptd; Sph; Spd; P (Phosphoric residue) derivatives of (a), (b), (c), (d), (e), (f);
   h) (a), (b), (c), (d), (e), (f), (g) salts (gum, gummi, and the like);
   i) compounds of formula:

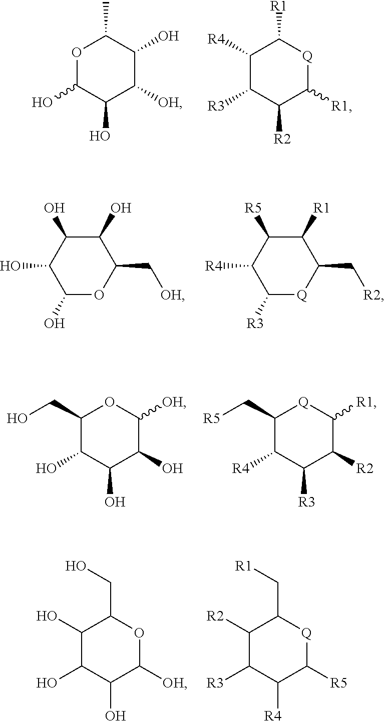

and structural isomers, anomers thereof; and stereoisomers thereof; and the like; wherein R₁-R₅ is H, halogen, F, Cl, Br, I, OH, Me, Et, Ac, OMe, OEt, OAc, OR^{A}, N(R^{A})₂, N₃, CN, NO₂, S(O)₇R^{A}, (C₁-C₁₅)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, —C(═O)R^{A}, —C(═O)OR^{A}, —C(═O)NR^{A}R^{B}, —C(═O)SR^{A}, —S(O)R^{A}, —S(O)₂R^{A}, S(O)(OR^{A}), —S(O)₂ (OR^{A}), —SO₂NR^{A}R^{B}, (C₆-C₂₀)aryl(C₁-C₈)alkyl, —PO(OH)₂—PO(OH)OPO(OH)₂, —PO(OH)OPO(OH)O PO(OH)₂, —PO(OR^{A})₂, —POR^{A}R^{B}, phenyl, 1-naphthyl, 2-naphthyl, benzyl, (C₃-C₆)cycloalkyl, —CH₂—(C₃-C₆)cycloalkyl, —O—(C₁-C₈) alkyl, —O-benzyl, —O—CH₂—(C₃-C₆)cycloalkyl, CF₃, C(halogen)₃, —C(=Q)R^{A}, —C(=Q)OR^{A}, —C(=Q)N(R^{A}), —NR^{A}R^{B}, —⁺N(R^{A})₃, —SR^{A}, —S(O)R^{A}, —S(O)₂R^{A}, —S(O)(OR^{A}), —S(O)₂(OR^{A}), —OC(=Q)R^{A}, —OC(=Q)OR^{A}, —OC(=Q)(N(R^{A})₂), SC(=Q)R^{A}, —SC(=Q)OR^{A}, —SC(=Q)(N(R^{A})₂), —N(R^{A})C(=Q)R^{A}, —N(R^{A})C(=Q)OR^{A}, —N(R^{A})C(=Q)N(R^{A})₂, —SO₂NR^{A}₂, —CN, —N₃, —NO₂, —OR^{A}, NR^{A}R^{B}, N(R^{A})OR^{B}, NR^{A}NR^{B}, N₃, NO, NO₂, CHO, CN, —CH(═NR^{A}), —CH═NNHR^{A}, —CH═N(OR^{A}), —CH(OR^{A})₂, —C(═O)NR^{B}R^{A}, —C(═S)NR^{B}R^{A}, —C(═O)OR^{A}, (C₆-C₂₀)optionally substituted aryl, optionally substituted heteroaryl, —C(═O)(C₁-C₈)alkyl, —S(O)_{z}(C₁-C₈)alkyl, SR^{A}, or selected from radicals obtained by removal of a hydrogen atom from the compounds shown in FIG. 8;
wherein Q is O, S, NR^{A}, ⁺N(O)(R^{A}), N(OR^{A}), ⁺N(O)(OR^{A}), or N—NR^{A}₂;
wherein R^{A}, R^{B} is H, halogen, F, Cl, Br, I, OH, Et, Me, Ac, OR^{C}, N(R^{C})₂, N₃, CN, NO₂, S(O)_{z}R^{C}, (C₁-C₁₅)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, —C(═O)R^{C}, —C(═O)OR^{C}, —C(═O)NR^{C}R^{C}, —C(═O)SR^{C}, —S(O)R^{C}, —S(O)₂R^{C}, —S(O)(OR^{C}), —S(O)₂ (OR^{C}), —SO₂NR^{C}R^{C}, (C₆-C₂₀)aryl(C₁-C₈)alkyl, —PO(OH)₂, —PO(OH)OPO(OH)₂, —PO(OH)OPO(OH)OPO(OH)₂, —PO(OR^{C})₂, —POR^{C}R^{C}, phenyl, 1-naphthyl, 2-naphthyl, benzyl, (C₃-C₆)cycloalkyl, —CH₂—(C₃-C₆)cycloalkyl, —O—(C₁-C₈) alkyl, —O-benzyl, —O—CH₂—(C₃-C₆)cycloalkyl, CF₃, C(halogen)₃, —C(O) R^{C}, —C(O)OR^{C}, —C(O)N(R^{C}), NR^{C}R^{C}, —⁺N(R^{C})₃, —SR^{C}, —S(O) R^{C}, —S(O)₂R^{C}, —S(O)(OR^{C}), —S(O)₂(OR^{C}), OC(O) R^{C}, —OC(O)OR^{C}, —OC(O)(N(R^{C})₂), —SC(=Q)R^{C}, —SC(=Q)OR^{C}, —SC(=Q)(N(R^{C})₂), —N(R^{C})C(=Q)R^{C}, —N(R^{C})C(=Q)OR^{C}, —N(R^{C})C(=Q)N(R^{C})₂, —SO₂NR^{C}₂, —CN, —N₃, —NO₂, —OR^{C}, NR^{C}R^{C}, N(R^{C})OR^{C}, NR^{C}NR^{C}, N₃, NO, NO₂, CHO, CN, —CH(═NR^{C}), —CH═NNHR^{C}, —CH═N(OR^{C}), —CH(OR^{C})₂, —C(═O)NR^{C}R^{C}, —C(═S)NR^{C}R^{C}, —C(═O)OR^{C}, (C₆-C₂₀)optionally substituted aryl, optionally substituted heteroaryl, —C(═O)(C₁-C₈)alkyl, —S(O)_{z}(C₁-C₈)alkyl, or SR^{C};
wherein R^{C} is H, OH, F, Cl, Br, I, Me, Et, Ac, OMe, OEt, or OAc, or selected from radicals obtained by removal of a hydrogen atom from the compounds shown in FIG. 8;
wherein z is from 1 to 10;
and structural isomers thereof; and stereoisomers thereof; and the like;
D2) glycated (O-, N-, P-, C-, S-linked, and the like) derivatives of (D1);
D3) (D1) or (D2) binded (O-, N-, P-, C-, S-linked, and the like) to any Protein, Peptide, Aptamer, Antibodie, Affibody, Avimer or Nanobody;
and wherein bonds may be covalent bonds, α1-1, α1-2, α1-3, α1-4, α1-5, α1-6, α1-7, α1-8, α2-1, α2-2, α2-3, α2-4, α2-5, α2-6, α2-7, α2-8, α2-9, α6-6, β1-1, β1-2, β1-3, β1-4, β1-5, β1-6, β1-7, β1-8, β1-9, β2-1, β2-2, β2-3, β2-4, β2-5, β2-6, β2-7, β2-8, β3-3 α1-2, and the like, αq-w, βq-w wherein q,w from 1 to 9;
E) wherein [HOST]ₙ is [X]ₙ wherein X is selected from:

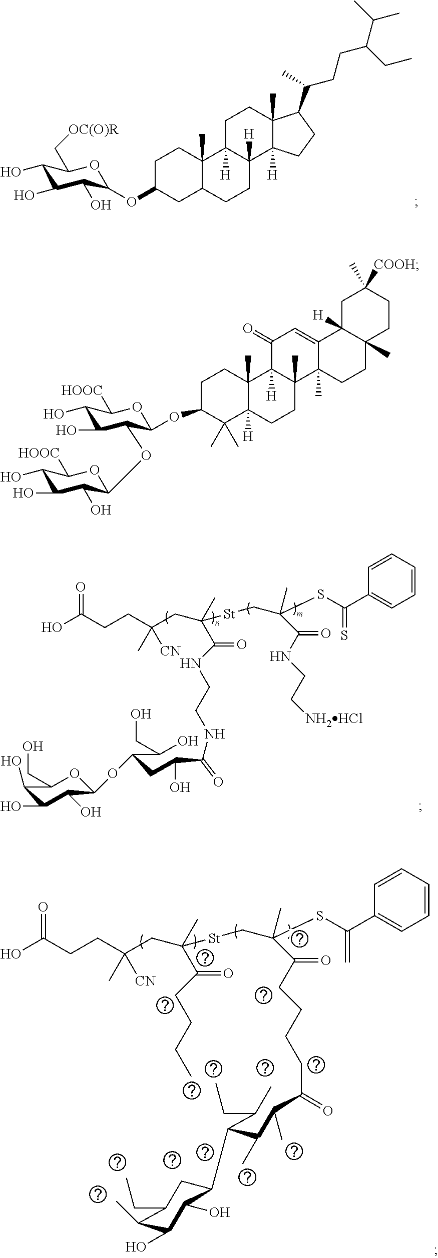

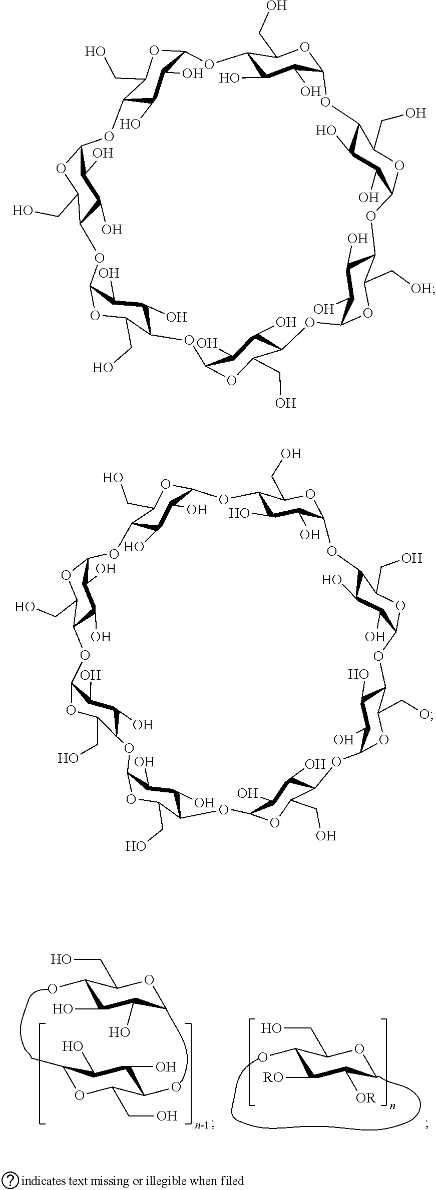

wherein R described in [0091] (B).
F) glycated derivatives, chemical or physical derivatives of A-E, structural isomers thereof; and stereoisomers thereof; and the like.

In another embodiment, provided are the non-covalent active pharmaceutical ingredient-carrier complex wherein the carrier is selected from:
A. Polymers and oligomers, predominantly organic polymers and oligomers, predominantly water-soluble organic polymers and oligomers, natural polymers and oligomers, biopolymers and biooligomers even more predominantly polysaccharides, homopolysaccharides, heteropolysaccharides, oligosaccharides, glycoconjugates, glycoproteins (O-, N-, P-, C-, S-linked), neoglycoproteins, glycolipids, glycopeptides, proteoglycans, peptidoglycans, glycolipids, glycosides, lipopolysaccharides, hemicelluloses, storage polysaccharides, sulfated polysaccharides and oligosaccharides, pectins, gums, mucilages, which may be (but are not exclusive to):
   A.1. Polyelectrolites and non-polyelectrolites;
   A.2. Polymers and oligomers, which can be obtained from animal, nonanimal, natural (i.e., microbiological or plant) sources:
      a) from plants, higher plants (non-limiting examples): Starch; Cellulose; Guar gum; Gum arable; Locust beanvgum; Pectin; Acacia gum; Gum arabic; Arabinogalactan; Costus glucans; Xylan; Glucomannan; Xyloglucan; Inulin; beta-glucan; Guar gum; Karaya gum; Tara gum; Fenugreek gum; Locust bean gum; Tragacanth gum; Cassia tor a gum; and the like;
      b) from mushrooms (non-limiting examples): Polysaccharide-K (Krestin, PSK), beta-glucans, Polysaccharide peptide (PSP); and the like;
      c) from Algae (non-limiting examples): Alginates; Galactans; Carrageenan; Alginate; Agar; Carrageenan; Fucoidan; Ulvan; Laminarin; Angelan; Porphyran; Spirulan; Agarose; Rhodymenan; and the like;
      d) from animal origin (non-limiting examples): Glycosaminoglycans(GAGs); Hyaluronic acid; Glycogen; Hyaluronan; Chitin; Chitosan; Heparin; Chondroitin sulphate; Keratan sulphate; Dermatan sulphate; Asialofetuin; fetuin; and the like;
      e) from microbial origin (non-limiting examples): Dextran; Gellan gum; Pullulan; Xanthan gum; Curdlan; Scleroglucan; Pullulan; Dextran; Xanthan; Levan; Gellan; Emulsan; Schizophyllan; Glucuronan; Succinoglycan; Nigcran; and the like;
   A.3. Hemicelluloses, which can be arabanes, arabinans, galactans (galactosans), glucans, xylans, mannans, fructans, xyloglucans, arabinogalactans, arabinoxylans, glucomannans, galactomannans, galactoglucomannans, beta-glucans, galactogens, and the like, their mixtures, but not excluding other hemicelluloses, and mixtures thereof;
   A.4. Sulfated polysaccharides and oligosaccharides which may be fucoidans, carrageenans or carrageenins, agaropectins, sea cucumber sulfated polysaccharides (SCSP), chondroitin sulfate, keratan sulfate, and the like, their mixtures, but not excluding other sulfated polysaccharides and oligosaccharides, and mixtures thereof;
   A.5. Oligosaccharides, fructooligosaccharides, galactooligosaccharides (oligogalactosyllactose, oligogalactose, oligolactose, transgalactooligosaccharides), xylooligosaccharides, isomaltooligosaccharides and the like, but not excluding other oligosaccharides and mixtures thereof;
   A.6. Polysaccharides and oligosaccharides, storage polysaccharides, sulfated polysaccharides and oligosaccharides, pectins, gums, mucilages which may be (but are not exclusive to others): polyuronic acids and esters; gum(s), gum arabic, xanthan gum, oat gum, gellan gum, guar gum, carob gum, karaya gum, dammar gum, gummiarabica, tara gum, ghatti gum, british gum, agar, agar-agar, tragakant gum, conjac gum, velan gum, dutan gum; galacturonic acid-based polysaccharides with side chains of rhamnose, arabinose, xylose and fructose and their salts (pectins, pectates, pectinates); pectin, pectins of beets, carrots, peppers, pumpkins, eggplant, sunflower, apples, quinces, cherries, plums, pears, citrus, zosterin; modified pectins, modified citrus pectin; acidic polysaccharides—i.e. e. polysaccharides containing carboxyl groups and/or phosphate groups and/or sulfuric ester groups; plantain husk, psyllium; soybean hemicellulose; galacturones, homogalacturones, polygalacturonic acids and their salts, rhamnogalacturonan, rhamnogalactans; calloses, laminarins, chrysolaminarins, curdlans; inulins, guars, dextrans, pullulans; agaroses; alginic acid and its salts alginates, propylene glycol alginate; arabin, arabic acid and its salts, and the like, but not excluding other, and mixtures thereof;
   A.7. Cellulose, cellulosic polymers, methylcellulose, ethylcellulose, hydroxypropylcellulose(s) (HPC), hydroxypropylmethylcellulose acetate succinate, hypromellose(s), hydroxypropylmethylcellulose(s) (HPMC), methylethylcellulose, ethylhydroxyethylcellulose, croscaramellose, carboxymethylcellulose and its salts; starch, starch(es), starch 1500G, soluble starch, modified starches, hydroxyethyl starch, cationic starch, acid-treated starch, alkaline modified starch, bleached starch, oxidized starch, enzyme treated starch, monostarch phosphate, distarch glycerol, distarch phosphate, phosphated distarch phosphate, acetylated distarch phosphate, starch acetate esterified with acetic anhydride, starch acetate esterified with vinyl acetate, acetylated distarch adipate, acetylated distarch glycerol, distarch glycerine, hydroxy propyl starch, hydroxy propyl distarch glycerine, hydroxy propyl distarch phosphate, hydroxy propyl distarch glycerol, starch sodium octenyl succinate, acetylated oxidised starch, and the like;
   A.8. Dextrin(s), maltodextrins, amylodextrins, polydextroses; amylopectin, amylose, glycogen; chitosan, chitins; pullulans, glucuronoaraboxylans, methyl-glucuronoaraboxylans, glycosaminoglycans, mucopolysaccharides, heparin/heparan sulfate, chondroitin sulfate, dermatan sulfate, keratan sulfate, hyaluronan, hyaluronic acid, and the like; and mixtures thereof;
   A.9. Glycated derivatives of these polymers and oligomers (O-, N-, P-, C-, S-linked);
   but not excluding other polymers and oligomers, biopolymers and biooligomers even more predominantly polysaccharides, homopolysaccharides, heteropolysaccharides, oligosaccharides, glycoconjugates, glycoproteins (O-, N-, P-, C-, S-linked), neoglycoproteins, glycolipids, glycopeptides, proteoglycans, peptidoglycans, glycolipids, glycosides, lipopolysaccharides, hemicelluloses, storage polysaccharides, sulfated polysaccharides and oligosaccharides, pectins, gums, mucilages and mixtures thereof;
B. Polymers, syntetic polymers, predominantly water-soluble polymers which may be (but are not limited to): polymers and copolymers formed from acrylic acid, methacrylic acid, and/or esters thereof; polymethacrylates, Eudragit and their salts; polyacrylamides; poly(amidoamine)s, poly(propyleneimine)s, polyethylene glycols; polyvinyl alcohol; polyvinylpyrrolidone; acrylic polymers, cellulose acetate phthalate(s), copovidone(s), ethyl oleate, glycerol derivatives, glyceryl triacetate, polyethylene glycol(s) (PEG), PEG derivatives, polymethacrylates, propylene glycol, propylene glycol derivatives, povidone(s), polyvinylpyrrolidone(s) (PVP), polyvinyl acetate phthalate(s) (PVAP), hypromellose acetate succinate(s) (HPMCAS), hydroxypropyl methylcellulose acetate succinate (HPMCAS), hypromellose phthalate(s) (HPMCP), cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, Avicel(s), Avicel PH101, Avicel PH102, Benecel(s), Brij(s), Brij 30, Brij 35, Capryol(s), Cavamax(s), Cavasol(s) and Cavitron(s) HPpCD cyclodextrins, Compritol 888 ATO, Cremophor(s), Cremophor EL, Cremophor RH40, DiCal Dihydrate, epoxidized palm oil (Epo), Eudragit(s), Eudragit E, Eudragit EPO, Eudragit L100, Eudragit L100-55, Eudragit 5100, Gelucire(s), Gelucire 44/14, HP-50 AAS-LF, HP-55 AAS-MF, HPMC(p-606), HPMC-E, HPMC-F, HPMC-K, HPMCAS-H, HPMCAS-L, HPMCAS-M, HPMCAS SDD, HPMCAS(AS-MG), HPMCAS-M SDDs, HPMCAS-MG, HPMCP (HP 55), HPMCP-HP55, HPMCPh, hypromellose phthalate HP-50, Imwitor(s), Imwitor 742, Klucel HPC, Kolhdon 17 PF, Kollicoat(s), Kollicoat IR, Kollicoat MAE, Kollicoat MAE 100, Kollicoat MAE 100P, Kollicoat Protect, Kollidon(s) (povidone(s)), Kollidon 12 pf, Kollidon 12/17PF, Kollidon 30, Kollidon 30/90, Kollidon 90, Kollidon CL-F, Kollidon CL-SF, Kollidon K30, Kollidon SR, Kollidon SR, Kollidon SR(PVAc), Kollidon V64/Fine, Kollidon VA 64, Kollidon VA 64 (copovidone), Kollidon VA64, Kolliphor(s), Kolliphor EL, Kolliphor EL/ELP, Kolliphor HS15, Kolliphor P 188, Kolliphor P 188/407, Kolliphor P 188/micro, Kolliphor P 407, Kolliphor P 407/micro, Kolliphor PS 20, Kolliphor PS 60, Kolliphor PS 80, Kolliphor RH 40, Kolliphor SLS, Kolliphor SLS/fine, Kollisolv(s), Kollisolv GTA, Kollisolv PEG 1450, Kollisolv PEG 300, Kollisolv PEG 3350, Kollisolv PEG 400, Kollisolv PEG E 300, Kollisolv PEG E 400, Kollisolv PEG grades (polyethylene glycol), Kolliwax(s), Kolliwax GMS II, Kolliwax SA, Labrasol(s), Lactose 310 Mono, Lactose FF316, Laurogucol(s), Maisine(s), Miglyol(s), Myrj(s), Myrj 52, PEG 1000, PEG 10000, PEG 1500, PEG 2000, PEG 20000, PEG 3000, PEG 400, PEG 4000, PEG 600, PEG 6000, PEG 800, PEG 8000, Pharmacoat(s), PVP K-12, PVP K-120, PVP K-15, PVP K-17, PVP K-30, PVP K-60, PVP K-90, PVP SDD, PVP VA64 SDDs, PVP-VA, PVP-VA 64, PVP-VA SDD, Palm stearin based polyesteramide (PSPEA), Peceol(s), pectin(s), Plasdone(s), Plasdone K povidone, Plasdone K-12 povidone, Plasdone K-29/32 povidone, Plasdone K-90 povidone, Plasdone S, Plasdone S-630 copovidone, poly(2-ethyl-2-oxazoline), poly(ethylene oxide) (PEO) (3400, 10000, 20000), polyoxyethylene stearate, Shin-Etsu AQOAT(s), Soluplus(es), Solutol(s), sucrose laurate, tocopheryl PEG 1000-succinate (TPGS), vitamin E TPGS, d-α-tocopherol polyethylene glycol 1000 succinate (TPGS), Isomalt (Galen IQ 810), galactosylated Polymers; galactosylated ciclodextrins; cationic glycopolymers; block and statistical carbohydrate-based galactosylated copolymers; galactosylated albumin(s), galactosylated bovine serum albumin; Statistical Cationic Glycopolymer of 2-aminoethyl methacrylamide (AEMA) and 2-lactobionamidoethyl methacrylamide (LAEMA) P(AEMA n-st-LAEMA m) (m,n from 1 to 1000); Cationic Block Glycopolymer of 2-aminoethyl methacrylamide (AEMA) and 2-lactobionamidoethyl methacrylamide; p-Vinylbenzyl galactoside (VBG); galactosylated N-3-guanidinopropyl methacrylamide-co-poly (ethylene glycol) methacrylate copolymers; galactosylated amphiphilic graft copolymer (PHEA-g-BIB-pButMA-g-PEG-GAL); (cholesteryloxycarbonylamino) ethylamine-α,β-polyasparthydrazied (CHE-PAHy-Lacs); PLGA-di-GAL; and the like, and others which are described in the scientific and patent literature (for example: Macro-Glycoligands. Methods and Protocols, Editors: Xue-Long Sun); and the like; but not excluding other synthetic polymers and mixtures thereof;
C. Copolymers is made from monomers of the polymers listed in paragraph A, B including alternating copolymers, random copolymers, block copolymers, graft copolymers, cross-linked modifications which may be (but are not limited to): methacrylic acid and ethyl acrylate copolymers; methacrylic acid copolymers; vinylpyrrolidone-vinyl acetate copolymers (PVPVA); polyvinylpolypyrrolidone (PVPP); PVA-PEG graft co-polymers; HPMC and PVA-PEG grafted copolymers; polyvinylpyrrolidone-arabinogalactan copolymers; grafted polyvinylpyrrolidone-arabinogalactan copolymers; the graft copolymer has a) poly(vinyl acetate) and/or poly (vinyl alcohol) and/or poly(vinyl chloride) and poly(vinyl ester) on b) a polymer chain of polyethylene glycols, polyalkylene glycols, polypropylene glycols, polyisobutylene glycols or polymethylpentene glycols; graft copolymer polyvinyl acetate and/or hydrolysed polyvinyl acetate (polyvinyl alcohol) groups on a polyalkylene oxide (preferably polyethylene oxide); vinylpyrrolidone-vinyl acetate copolymers; vinylpyrrolidone-vinyl acetate copolymer-64+; vinylpyrrolidone-vinyl acetate VA 64; polymethacrylate-based copolymers includes anionic, cationic, and neutral copolymers based on methacrylic acid and methacrylic/acrylic esters their salts, esters or other derivatives and mixtures thereof and the like;
D. Polymers and oligomers, copolymers, biopolymers (natural, obtained by chemical or physical modification of natural polymers (chemical or physical derivatives), or synthesized specifically) that under at least one of the following conditions:
   1. co-occurring in physiologic body fluids, plasma, intercellular fluid together with APIs;
   2. by non-covalent binding to API;
   3. by covalent binding to API into a conjugate;
result in at least one of the following:
   a. an increase in the permeability of the API, API-carrier system (solution, non-covalent complex, conjugate, and the like);
   b. an increase in the concentration of API, API-carrier system in cells and tissues;
for at least one of the following reasons:
   i. inhibition or modulation of multidrug resistance (MDR), predominantly mediated by efflux transporters of the ATP-binding cassette (ABC) superfamily including P-glycoprotein (P-gp);
   ii. induction, inhibition or modulation of CYP1, CYP2, CYP3 enzymes (non limiting examples: CYP1A1, 1A2, 2A6, 2B6, 2C8, 2C9, 2C19, 2D6, 2E1 or 3A4 enzymes);
   iii. due to a change in the structure of the cell membrane, change in lipid biolayer properties after the incorporation of carrier substance molecules (or carrier molecular part of NCC or conjugate) into this membrane or due adhesion to this membrane;
   iv. due to changes in charge, hydrophilicity, hydrophobicity or other biophysical properties of the non-covalent complex, or conjugate due to which changes in the transmembrane (passive, active) or paracellular transport of the non-covalent complex, or conjugates;
   v. for other reasons e.g., by the introduction or presence in the carrier of targeted fragments capable of selectively interacting with the receptor, cell surface receptors, membrane receptors, transmembrane receptors. Low molecular weight compounds, peptides, aptomers, and the like are used as vector molecules and are described in the patent or scientific literature; or by other reasons described in the patent or scientific literature.
E. Other carriers:
   E1. Macrocyclic hosts which may be (but are not exclusive to others): cyclodextrins, cucurbit[n]urils, and calix[n]arenes, pillarenes, crown ethers, cyclophanes, cryptands;
   E2. Acids and salts based on these acids which may be (but are not limited to): citric acid, tartaric acid, succinic acid, phosphoric acid, aminoacids, acetate(s), sodium acetate, alginate(s), sodium alginate, glycyrrhizic acid and their salts, and mixtures thereof; 8
   E3. Polyols which may be (but are not limited to): ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and mixtures thereof;
   E4. Monosaccharides, disaccharides, trisaccharides, tetrasaccharides, pentaccharides, hectaccharides and the like mixtures thereof;
   E5. Surfactants which may be (but are not limited to): anionic surfactants contained anionic functional groups at their head, such as sulfate, sulfonate, phosphate, carboxylates, carboxylate salts; alkyl sulfates; alkyl-ether sulfates; carboxylate-based fluorosurfactants; cationic surfactants; primary, secondary, or tertiary amines; permanently charged quaternary ammonium salts; zwitterionic (amphoteric) surfactants; zwitterionic surfactants which cationic part is based on primary, secondary, or tertiary amines or quaternary ammonium cations; sultaines; betaines; phospholipids; zwitterionic surfactants of the tertiary amine oxides structural type; non-ionic surfactants; ethoxylates; fatty alcohol ethoxylates; alkylphenol ethoxylates (apes or apeos); fatty acid ethoxylates; special ethoxylated fatty esters and oils; ethoxylated amines and/or fatty acid amides; terminally blocked ethoxylates; fatty acid esters of polyhydroxy compounds; fatty acid esters of glycerol; fatty acid esters of sorbitol; fatty acid esters of sucrose; alkyl polyglucosides; ammonium lauryl sulfate; sodium lauryl sulfate; sodium dodecyl sulfate; sodium laureth sulfate; sodium lauryl ether sulfate; sodium myreth sulfate; docusate (dioctyl sodium sulfosuccinate) and their salts; perfluorooctanesulfonate (pfos); perfluorobutanesulfonate; alkyl-aryl ether phosphates; alkyl ether phosphates; sodium stearate; sodium lauroyl sarcosinate; perfluorononanoate; perfluorooctanoate; octenidine dihydrochloride; cetrimonium bromide (ctab); cetylpyridinium chloride (cpc); benzalkonium chloride (bac); benzethonium chloride (bzt); dimethyldioctadecylammonium chloride; dioctadecyldimethylammonium bromide (dodab); chaps (3-[(3-cholamidopropyl)dimethylammonio]-1-propanesulfonate); cocamidopropyl hydroxysultaine; cocamidopropyl betaine; phosphatidylserine; phosphatidylethanolamine; phosphatidylcholine; sphingomyelins; lauryldimethylamine oxide; myristamine oxide; narrow-range ethoxylates; octaethylene glycol monododecyl ether; pentaethylene glycol monododecyl ether; nonoxynols; triton x-100; polyethoxylated tallow amine; cocamide monoethanolamine; cocamide diethanolamine; poloxamers; glycerol monostearate; glycerol monolaurate; sorbitan monolaurate; sorbitan monostearate; sorbitan tristearate; tween(s), tween 20; tween 40; tween 60; tween 80; decyl glucoside; span(s), span 20, span 40, span 80; lauryl glucoside; octyl glucoside; poloxamer(s), poloxamer 188, poloxamer 407, polyoxyethylene stearate, myrj 52, deoxycholic acid, bile acids, pluronic(s), pluronic F-127, pluronic P85, pluronic f68, gelucire(s), gelucire 44/14, lecithins, polysorbates, polysorbate 80, plasdone-s630, pluronic-f68, inutec sp1, compritol 888 ato, tocopherol polyethylene glycol succinate, polyoxyethylated castor oil, polyoxyethylated glycerides, lauroyl macroglycerides, and mono- and di-fatty acid esters of low molecular weight polyethylene glycols; and mixtures thereof;
F. Methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in A-E, and their esters, salts, and any other chemical derivatives, and mixtures thereof or/and which are described in the scientific and patent literature; Alternatively, dextran or poly-L-lysine can be attached to the gum arabic carrier to provide an increased number of sites of attachment for the therapeutic agent.
G. Any combination of substances specified in items A-F.

In another embodiment, provided are the non-covalent complex when dissolved in a physiologically acceptable solvent other than water.

In another embodiment, provided are the non-covalent when non-covalent complex is inclusion complex or supramolecular complex.

Provided are the methods (co-treating methods/solid dispersion techniques) for modifying the properties of an active pharmaceutical ingredient (API) by creating a molecular/solid dispersion, solid phase inclusion or supramolecular complex (both of Formula II) using API, host substance (and, if necessary, other compounds) with properties of increased bioavailability and/or permeability and using Formula II compounds obtained by this methods (or aqueous solutions or suspensions thereof) to treat a disease in humans or animal or mammal or bird and/or to manufacture a pharmaceutical dosage form to treat disease in a human or animal or mammal or bird in need thereof (by administering a therapeutically effective amount), at a dosage ranging from 0.0001 ng/kg to 100000 mg/kg (calculated for pure API)
wherein active pharmaceutical ingredient is selected from:
   Milbemycins; Milbemycin A3; Milbemycin A4; Milbemycin D; Milbemycin B2; Milbemycin B3; Milbemycin G; Nemadectins; Nemadectin; Meilingmycins; Meilingmycin A; Milbemectins; Milbemectin; Milbemycin oxime; Moxidectins; Moxidectin; Doramectins; Doramectin; Selamectins; Selamectin; Eprinomectins; Eprinomectin B1a; Eprinomectin B1b; Emamectins; Emamectin B1a; Emamectin B1b; Tenvermectins; Tenvermectin B; Tenvermectin A; Avermectins; Avermectin A1a; Avermectin A1b; Avermectin A2a; Avermectin A2b; Avermectin B1a; Avermectin B1b; Avermectin B2a; Avermectin B2b; Ivermectins; Ivermectin B1a; Ivermectin B1b; Ivermectin A1a; Ivermectin A1b; Fenbendazole; Flubendazole; Thiabendazole; Oxfendazole; Triclabendazole; Mebendazole; Oxyclozanide; Niclosamide; Nitazoxanide; Colchicine; Artemisinin; Quercetin; Curcumin; Resveratrol; thymoquinone; Pyrvinium; Albendazole sulfoxide; Albendazole or compounds selected from those listed in from FIG. 12 to FIG. 15; or compounds (API, AAPI, AANPI) selected from those listed in [0110], [0111], [0114]; or derivatives thereof such as racemate, enantiomer, diastereomer, tautomer, polymorph, pseudopolymorph, hydride or solvate thereof; or a pharmaceutically acceptable salt or ester or ether thereof; or prodrug thereof; or methabolite thereof; or aglycone thereof; or derivatives which may be obtained by gene replacement processes in genetically engineered strains, or/and by fermentation, or/and by chemical modifications and the like, which are described in the scientific and patent literature; or mixtures thereof;
wherein the host substance is selected from:
   A. Polymers and oligomers, predominantly organic polymers and oligomers, even more predominantly polysaccharides and oligosaccharides, hemicelluloses, storage polysaccharides, sulfated polysaccharides and oligosaccharides, pectins, gums, mucilages, which may be (but are not exclusive to):
      A.1. Hemicelluloses, which can be arabanes, arabinans, galactans (galactosans), glucans, xylans, mannans, fructans, xyloglucans, arabinogalactans, arabinoxylans, glucomannans, galactomannans, galactoglucomannans, beta-glucans, galactogens, their mixtures, but not excluding other hemicelluloses, and mixtures thereof;
      A.2. Sulfated polysaccharides and oligosaccharides which may be fucoidans, carrageenans or carrageenins, agaropectins, sea cucumber sulfated polysaccharides (SCSP), chondroitin sulfate, keratan sulfate, their mixtures, but not excluding other sulfated polysaccharides and oligosaccharides, and mixtures thereof;
      A.3. Polysaccharides and oligosaccharides, storage polysaccharides, sulfated polysaccharides and oligosaccharides, pectins, gums, mucilages which may be (but are not exclusive to others): polysaccharides based on glucose, dextrose, galactose, mannose, arabinose, rhamnose, sucrose, maltose, lactose and their uronic acids, which may be methoxylated or acetylated, and their salts (gum, gummi); polyuronic acids and esters; gum, xanthan gum, oat gum, gellan gum, guar gum, carob gum, karaya gum, dammar gum, gummiarabica, tara gum, ghatti gum, british gum, agar, agar-agar, tragakant gum, conjac gum, velan gum, dutan gum; galacturonic acid-based polysaccharides with side chains of rhamnose, arabinose, xylose and fructose and their salts (pectins, pectates, pectinates); pectin, pectins of beets, carrots, peppers, pumpkins, eggplant, sunflower, apples, quinces, cherries, plums, pears, citrus, zosterin; modified pectins, modified citrus pectin; acidic polysaccharides—i.e. e. polysaccharides containing carboxyl groups, phosphate groups and/or sulfuric ester groups; plantain husk, psyllium; soybean hemicellulose; galacturones, homogalacturones, polygalacturonic acids and their salts, rhamnogalacturonan, rhamnogalactans; calloses, laminarins, chrysolaminarins, curdlans; inulins, guars, dextrans, pullulans; agaroses, galacto-oligosaccharides (oligogalactosyllactose, oligogalactose, oligolactose or transgalactooligosaccharides), xylooligosaccharides, fructooligosaccharides, isomaltooligosaccharide; alginic acid and its salts alginates, propylene glycol alginate; arabin, arabic acid and its salts; cellulose, cellulosic polymers, methylcellulose, ethylcellulose, hydroxypropylcellulose(s) (HPC), hydroxypropylmethylcellulose acetate succinate, hypromellose(s), hydroxypropylmethylcellulose(s), (HPMC), methylethylcellulose, ethylhydroxyethylcellulose, croscaramellose, carboxymethylcellulose and its salts; starch, starch(es), starch 1500G, soluble starch, modified starches, hydroxyethyl starch, cationic starch, acid-treated starch, alkaline modified starch, bleached starch, oxidized starch, enzyme treated starch, monostarch phosphate, distarch glycerol, distarch phosphate, phosphated distarch phosphate, acetylated distarch phosphate, starch acetate esterified with acetic anhydride, starch acetate esterified with vinyl acetate, acetylated distarch adipate, acetylated distarch glycerol, distarch glycerine, hydroxy propyl starch, hydroxy propyl distarch glycerine, hydroxy propyl distarch phosphate, hydroxy propyl distarch glycerol, starch sodium octenyl succinate, acetylated oxidised starch; dextrin(s), maltodextrins, cyclodextrins, amylodextrins, polydextroses; amylopectin, amylose, glycogen; chitosan, chitins; pullulans, glucuronoaraboxylans, methyl-glucuronoaraboxylans, glycosaminoglycans, mucopolysaccharides, heparin/heparan sulfate, chondroitin sulfate, dermatan sulfate, keratan sulfate, hyaluronan, hyaluronic acid and mixtures thereof, but not excluding other polysaccharides and oligosaccharides, hemicelluloses, stored polysaccharides, sulfated polysaccharides and oligosaccharides, mucilages and mixtures thereof;
      A.4. Macrocyclic hosts which may be (but are not exclusive to others): cyclodextrins, cucurbit[n]urils, and calix[n]arenes, pillarenes, crown ethers, cyclophanes, cryptands;
   B. Substances that may contain in significant amounts (more than 5% wt.) polysaccharides and oligosaccharides, hemicelluloses, storage polysaccharides, sulfated polysaccharides and oligosaccharides, pectins, gums, mucilages, which may be (but are not limited to) plants or algae or animal or fungi, parts of plants or algae or animal or fungi, processed plants or algae or animal or fungi, processed parts of plants or algae or animal or fungi containing in significant amounts the substances specified in paragraph A, and mixtures thereof. A frequent but non-limiting example is dried brown or red algae such as kelp or focus;
   C. Syntetic polymers, predominantly water-soluble polymers which may be (but are not limited to): polymers and copolymers formed from acrylic acid, methacrylic acid, and/or esters thereof; polymethacrylates, Eudragit and their salts; polyacrylamides; poly(amidoamine)s, poly(propyleneimine)s, polyethylene glycols; polyvinyl alcohol; polyvinylpyrrolidone; acrylic polymers, cellulose acetate phthalate(s), copovidone(s), ethyl oleate, glycerol derivatives, glyceryl triacetate, polyethylene glycol(s) (PEG), PEG derivatives, polymethacrylates, propylene glycol, propylene glycol derivatives, povidone(s), polyvinylpyrrolidone(s) (PVP), polyvinyl acetate phthalate(s) (PVAP), hypromellose acetate succinate(s) (HPMCAS), hydroxypropyl methylcellulose acetate succinate (HPMCAS), hypromellose phthalate(s) (HPMCP), cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, Avicel(s), Avicel PH101, Avicel PH102, Benecel(s), Brij(s), Brij 30, Brij 35, Capryol(s), Cavamax(s), Cavasol(s) and Cavitron(s) HPpCD cyclodextrins, Compritol 888 ATO, Cremophor(s), Cremophor EL, Cremophor RH40, DiCal Dihydrate, epoxidized palm oil (Epo), Eudragit(s), Eudragit E, Eudragit EPO, Eudragit L100, Eudragit L100-55, Eudragit S100, Gelucire(s), Gelucire 44/14, HP-50 AAS-LF, HP-55 AAS-MF, HPMC(p-606), HPMC-E, HPMC-F, HPMC-K, HPMCAS-H, HPMCAS-L, HPMCAS-M, HPMCAS SDD, HPMCAS(AS-MG), HPMCAS-M SDDs, HPMCAS-MG, HPMCP (HP 55), HPMCP-HP55, HPMCPh, hypromellose phthalate HP-50, Imwitor(s), Imwitor 742, Klucel HPC, Kolhdon 17 PF, Kollicoat(s), Kollicoat IR, Kollicoat MAE, Kollicoat MAE 100, Kollicoat MAE 100P, Kollicoat Protect, Kollidon(s) (povidone(s)), Kollidon 12 pf, Kollidon 12/17PF, Kollidon 30, Kollidon 30/90, Kollidon 90, Kollidon CL-F, Kollidon CL-SF, Kollidon K30, Kollidon SR, Kollidon SR, Kollidon SR(PVAc), Kollidon V64/Fine, Kollidon VA 64, Kollidon VA 64 (copovidone), Kollidon VA64, Kolliphor(s), Kolliphor EL, Kolliphor EL/ELP, Kolliphor HS15, Kolliphor P 188, Kolliphor P 188/407, Kolliphor P 188/micro, Kolliphor P 407, Kolliphor P 407/micro, Kolliphor PS 20, Kolliphor PS 60, Kolliphor PS 80, Kolliphor RH 40, Kolliphor SLS, Kolliphor SLS/fine, Kollisolv(s), Kollisolv GTA, Kollisolv PEG 1450, Kollisolv PEG 300, Kollisolv PEG 3350, Kollisolv PEG 400, Kollisolv PEG E 300, Kollisolv PEG E 400, Kollisolv PEG grades (polyethylene glycol), Kolliwax(s), Kolliwax GMS II, Kolliwax SA, Labrasol(s), Lactose 310 Mono, Lactose FF316, Laurogucol(s), Maisine(s), Miglyol(s), Myrj(s), Myrj 52, PEG 1000, PEG 10000, PEG 1500, PEG 2000, PEG 20000, PEG 3000, PEG 400, PEG 4000, PEG 600, PEG 6000, PEG 800, PEG 8000, Pharmacoat(s), PVP K-12, PVP K-120, PVP K-15, PVP K-17, PVP K-30, PVP K-60, PVP K-90, PVP SDD, PVP VA64 SDDs, PVP-VA, PVP-VA 64, PVP-VA SDD, Palm stearin based polyesteramide (PSPEA), Peceol(s), pectin(s), Plasdone(s), Plasdone K povidone, Plasdone K-12 povidone, Plasdone K-29/32 povidone, Plasdone K-90 povidone, Plasdone S, Plasdone 5-630 copovidone, poly(2-ethyl-2-oxazoline), poly(ethylene oxide) (PEO) (3400, 10000, 20000), polyoxyethylene stearate, Shin-Etsu AQOAT(s), Soluplus(es), Solutol(s), sucrose laurate, tocopheryl PEG 1000-succinate (TPGS), vitamin E TPGS, d-α-tocopherol polyethylene glycol 1000 succinate (TPGS), Isomalt (Galen IQ 810), but not excluding other synthetic polymers and mixtures thereof;
   D. Polyols which may be (but are not limited to): ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and mixtures thereof;
   E. Saccharides which may be glucose, dextrose, galactose, mannose, arabinose, rhamnose, sucrose, maltose, lactose, ribose, and mixtures thereof;
   F. Surfactants which may be (but are not limited to): anionic surfactants contained anionic functional groups at their head, such as sulfate, sulfonate, phosphate, carboxylates, carboxylate salts; alkyl sulfates; alkyl-ether sulfates; carboxylate-based fluorosurfactants; cationic surfactants; primary, secondary, or tertiary amines; permanently charged quaternary ammonium salts; zwitterionic (amphoteric) surfactants; zwitterionic surfactants which cationic part is based on primary, secondary, or tertiary amines or quaternary ammonium cations; sultaines; betaines; phospholipids; zwitterionic surfactants of the tertiary amine oxides structural type; non-ionic surfactants; ethoxylates; fatty alcohol ethoxylates; alkylphenol ethoxylates (apes or apeos); fatty acid ethoxylates; special ethoxylated fatty esters and oils; ethoxylated amines and/or fatty acid amides; terminally blocked ethoxylates; fatty acid esters of polyhydroxy compounds; fatty acid esters of glycerol; fatty acid esters of sorbitol; fatty acid esters of sucrose; alkyl polyglucosides; ammonium lauryl sulfate; sodium lauryl sulfate; sodium dodecyl sulfate; sodium laureth sulfate; sodium lauryl ether sulfate; sodium myreth sulfate; docusate (dioctyl sodium sulfosuccinate) and their salts; perfluorooctanesulfonate (pfos); perfluorobutanesulfonate; alkyl-aryl ether phosphates; alkyl ether phosphates; sodium stearate; sodium lauroyl sarcosinate; perfluorononanoate; perfluorooctanoate; octenidine dihydrochloride; cetrimonium bromide (ctab); cetylpyridinium chloride (cpc); benzalkonium chloride (bac); benzethonium chloride (bzt); dimethyldioctadecylammonium chloride; dioctadecyldimethylammonium bromide (dodab); chaps (3-[(3-cholamidopropyl)dimethylammonio]-1-propanesulfonate); cocamidopropyl hydroxysultaine; cocamidopropyl betaine; phosphatidylserine; phosphatidylethanolamine; phosphatidylcholine; sphingomyelins; lauryldimethylamine oxide; myristamine oxide; narrow-range ethoxylates; octaethylene glycol monododecyl ether; pentaethylene glycol monododecyl ether; nonoxynols; triton x-100; polyethoxylated tallow amine; cocamide monoethanolamine; cocamide diethanolamine; poloxamers; glycerol monostearate; glycerol monolaurate; sorbitan monolaurate; sorbitan monostearate; sorbitan tristearate; tween(s), tween 20; tween 40; tween 60; tween 80; decyl glucoside; span(s), span 20, span 40, span 80; lauryl glucoside; octyl glucoside; poloxamer(s), poloxamer 188, poloxamer 407, polyoxyethylene stearate, myrj 52, deoxycholic acid, bile acids, pluronic(s), pluronic F-127, pluronic P85, pluronic f68, gelucire(s), gelucire 44/14, lecithins, polysorbates, polysorbate 80, plasdone-s630, pluronic-f68, inutec sp1, compritol 888 ato, tocopherol polyethylene glycol succinate, polyoxyethylated castor oil, polyoxyethylated glycerides, lauroyl macroglycerides, and mono- and di-fatty acid esters of low molecular weight polyethylene glycols; and mixtures thereof;
   G. Acids and salts based on these acids which may be (but are not limited to): citric acid, tartaric acid, succinic acid, phosphoric acid, aminoacids, acetate(s), sodium acetate, alginate(s), sodium alginate, glycyrrhizic acid and their salts, and mixtures thereof;
   H. Oxides and salts based on these oxides which may be (but are not limited to): silicon dioxide, silicates, titanium dioxide, and mixtures thereof;
   I. Copolymers of the polymers listed in paragraph A, C including alternating copolymers, random copolymers, block copolymers, graft copolymers, cross-linked modifications which may be (but are not limited to): methacrylic acid and ethyl acrylate copolymers; methacrylic acid copolymers; vinylpyrrolidone-vinyl acetate copolymers (PVPVA); polyvinylpolypyrrolidone (PVPP); PVA-PEG graft co-polymers; HPMC and PVA-PEG grafted copolymers; grafted polyvinylpyrrolidone-arabinogalactan copolymers; the graft copolymer has a) poly(vinyl acetate) and/or poly (vinyl alcohol) and/or poly(vinyl chloride) and poly(vinyl ester) on b) a polymer chain of polyethylene glycols, polyalkylene glycols, polypropylene glycols, polyisobutylene glycols or polymethylpentene glycols; graft copolymer polyvinyl acetate and/or hydrolysed polyvinyl acetate (polyvinyl alcohol) groups on a polyalkylene oxide (preferably polyethylene oxide); vinylpyrrolidone-vinyl acetate copolymers; vinylpyrrolidone-vinyl acetate copolymer-64+; vinylpyrrolidone-vinyl acetate VA 64; polymethacrylate-based copolymers includes anionic, cationic, and neutral copolymers based on methacrylic acid and methacrylic/acrylic esters their salts, esters or other derivatives and mixtures thereof;
   J. Carriers listed in [0092] and [0093];
   K. Methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in A-J, and their esters, salts, and any other chemical derivatives, and mixtures thereof;
   L. Any combination of substances specified in items A-K;
wherein co-treating method (solid dispersion preparation method) is selected from:
   a) mechanochemical method; grinding/milling with high energy stress method;
   b) mechanochemical method with solvent; grinding/milling with high energy stress and solvent method;
   c) media milling method;
   d) kneading method;
   e) hot-melt method/melting method/fusion method;
   f) hot-melt extrusion/hot-stage extrusion method;
   g) meltrex method;
   h) melt agglomeration method;
   i) high-pressure homogenization method;
   j) solvent evaporation method;
   k) spin-coated films method; electrostatic spinning, electrostatic blowing, electrospraying film casting;
   l) spray-drying method;
   m) supercritical fluid (SCF) process method;
   n) cryogenic techniques method;
   o) lyophilization/freeze-drying technique method;
   p) spray freezing onto cryogenic fluids method;
   q) spray freezing into cryogenic liquids (SFL) method;
   r) spray freezing into vapor over liquid (SFV/L) method;
   s) ultra-rapid freezing method;
   t) precipitation/co-precipitation method;
   u) microwave irradiation method;
   v) energy input method;
   w) heat/shear energy input method;
   x) gel entrapment technique;
   y) a method comprising: contacting an active pharmaceutical ingredient and a matrix “host substance” to form a solid dispersion under conditions sufficient to form a solid dispersion;
   x) combined method.

In another embodiment, provided are the methods and the complex when instead of non-covalent complexes, conjugates (of Formula III) are administered, obtained by the methods described in this invention and in the patent and scientific literature.

### V. Combination Therapy/Therapeutic Combinations of NCC, SD, Conjugate

The compound of Formula I-III of the invention are also used in combination with other active ingredients. The compounds and compositions of the present invention are also intended for use in the general care of patients with disease, including nutrition and/or antifungal prophylaxis, antipyretics and analgesics, antiemetics (such as metoclopramide) and/or anti-diarrheals vitamin and mineral supplements (including vitamin K), anti-inflammatories (such as ibuprofen), pain medications, and medications for other common conditions in the patient population, such as antimalarials (including artemether and artesunate-lumefantrine combination therapy), typhoid or shigellosis.

It is also possible to combine any compound of the invention with one or more additional active therapeutic agents in a unitary dosage form for simultaneous or sequential administration to a patient. The combination therapy may be administered as simultaneous or sequential administration. In case of sequential administration the combination may be administered in two or more injections.

Co-administration of a compound of the invention with one or more other active therapeutic agents generally refers to the simultaneous or sequential administration of a compound of the invention and one or more other active therapeutic agents such that therapeutically effective amounts of the compound of the invention and one or more other active therapeutic agents are simultaneously present in the patient.

Co-administration comprises administering single doses of the compounds of the invention before or after administering single doses of one or more other active therapeutic agents, for example, administering the compounds of the invention within seconds, minutes or hours after administering one or more other active therapeutic agents. For example, a single dose of the compound of the invention may be administered first, followed by a single dose of one or more other active therapeutic agents within seconds or minutes. Alternatively, a single dose of one or more other therapeutic agents may be administered first, followed by a single dose of the compound of the invention within seconds or minutes. In some cases, it may be desirable to first administer a single dose of the compound of the invention and then, after a few hours (e.g., 1-12 hours), administer a single dose of one or more other active therapeutic agents. In other cases, it may be desirable to first administer a single dose of one or more other active therapeutic agents and then administer a single dose of the compound of the invention a few hours later (e.g., 1-12 hours).

Combination therapy can provide “synergism” and “synergy”, i.e., the effect achieved when the active ingredients are used together is greater than the sum of the effects occurring when the compounds are used separately. Synergistic effects can be achieved when the active ingredients are (1) combined and administered or delivered simultaneously in a combination drug; (2) delivered alternately or in parallel as separate drugs; or (3) by some other scheme. When drugs are alternated, a synergistic effect may be achieved when the compounds are administered or delivered sequentially, for example, in separate tablets, pills, or capsules, or when different injections are given in separate syringes. In general, in alternation, the effective dose of each active ingredient is administered sequentially, that is, serially, whereas in combination therapy the effective doses of two or more active ingredients are administered together. Synergistic effect means an cancer effect that exceeds the predicted purely additive effect of the individual compounds of the combination.

A kit comprising a compound of Formula I-IV is also provided. In some embodiments of the invention, individual kits are provided comprising a compound selected from the group of each of the formulas provided herein and each subgroup and embodiments thereof, including Formula II, Formula II, Formula I. In one aspect, the kit includes a compound of Formula I. Each of the individual kits described herein may include a label and/or instructions for administering the compound to treat a disease or condition in a subject (e.g., human) in need thereof. In some embodiments of the invention, the disease or condition is a “diseases of the invention”. In other embodiments of the invention, each individual kit may also contain instructions for administering additional medical agents in combination with a compound of Formula I to treat a disease or condition in a subject (e.g., human) in need thereof. In each of the embodiments presented herein, there is another embodiment in which the kit includes individual dosage units of the compound described herein. Examples of individual doses may include tablets, tablets, capsules, filled syringes or syringe cartridges, IV bags, etc., each containing a therapeutically effective amount of the compound in question. In some embodiments, the kit may contain a single dosage unit, and in other embodiments, the kit may contain multiple dosage units, such as the number of dosage units required for a particular regimen or period.

In certain embodiments, the product container may be a vial, jar, ampule, preloaded syringe, blister pack, tin, can, bottle, box, or intravenous bag.

Also, certain embodiments of the invention provide for the use of a compound selected from each of claims herein in the preparation of a medicament for use in the treatment of “diseases of the invention” in humans.

In another embodiment, the method of treating a “diseases of the invention” in a human in need thereof comprises administering a therapeutically effective amount of a pharmaceutical composition comprising an effective amount of a Formula I-III compound in combination with at least one additional therapeutic agent.

In another embodiment, a method of treating “diseases of the invention” in a person in need thereof includes administering a therapeutically effective amount of a pharmaceutical composition comprising an effective amount of compound of Formula I-III in combination with at least one additional therapeutic agent such as (but not limited to) intravenous or intramuscular forms of thiamine, vitamin C, vitamin D, glutanione, S-adenosylmethionine, cysteine, N-acetylcysteine.

In another embodiment, the method comprises administering a therapeutically effective amount of a combination pharmaceutical agent (of Formula I-III) comprising:
a) a first pharmaceutical agent—compound of Formula I-III (is 0.01-99.99% of the total weight of the Formula I-III), and
b) a second pharmaceutical agent/agents (AAPI, AAPI(s)) comprising at least one additional therapeutic agent active “diseases of the invention” of the Formula I-III component, or decreases the hepatotoxicity of the Formula I-III component, or increases the stability of the Formula I-III component (0.01-99.99% of the total weight of the composition) which may be (but are not limited to): niclosamide, nitazoxanide, chloroquine, lopinavir, ritonavir, hydroxychloroquine, vegetable or animal fats, ethyl alcohol, water, glycerin, propylene glycol, triethylene glycol, dimethylsulfoxide, solvents, betaine hydrochloride, caprilic acid, monolauric acid, monolaurin (glyceryl monolaurate), undecenoic acid (undecylenic acid), pau p'arco bark extract, cat's claw extract, garlic extract, black walnut shell extract, catalase enzyme, lipase, glucoamylase, pectinase, beta-glucanase, cellulase, alpha-galactosidase, amylase, invertase, xylanase, hemicellulase, or other enzymes, black cumin oil, nigella sativa, oregano oil, essential oils, lactoferrin, colloid silver, vitamin C, thiamine, benfothiamine, sulbutiamine, allithiamine, vitamin D, vitamin A, zinc salts, iodine, melatonin, phenofibrate, curcumin, quercetin, resveratrol, lysine, glycine, glutathione, N-acetylcysteine, cysteine, lipoic acid, S-adenosyl-L-methionine, milk thistle extract, grape seed extract, carnitine, antiandrogens, aspirin, bromhexine, budesonide, cannabidiol, colchicine, convalescent plasma, ensovibep, famotidine, favipiravir, fluvoxamine, iota-carrageenan, metformin, molnupiravir, bamlanivimab/etesevimab, bebtelovimab, casirivimab/imdevimab, sotrovimab, tixagevimab/cilgavimab, nitric oxide, paxlovid, peginterferon lambda, povidone-iodine, proxalutamide, camostat (camostat mesilate, camostat mesylate), foistar, Glycyrrhiza glabra, liquorice, glycyrrhizic acid, amantadine, artemisia annua, amodiaquine, artemisinin, rutin, hesperidin, hesperetin, luteolin, baricitinib, arbidol, umifenovir, fluoxetine, kaempferol, kaempferol 3-o-rutinoside, catechin, epigallocatechin, epigallocatechin gallate, epigallocatechin-3-gallate, myricetin, andrographolide, atorvastatin, indomethacin, nafamostat, nafamostat mesilate, nafamostat mesylate, omega 3, dha/epa, xylitol, xlear, ellagic acid, apn01, alunacedase alfa, soluble ace2, rhace2-apn01, azelastine, baicalin, cetylpyridinium chloride, estradiol, hydrogen peroxide, loratadine, methylene blue, monolaurin, propolis, sofosbuvir, darunavir, galidesivir, cepharanthine, hypericin, spironolactone, apigenin, emodin, apilimod, atovaquone, ciclesonide, daclatasvir, fenofibrate, fucoidan, hypochlorous acid, methotrexate, nicotine, pomegranate, punica granatum, punicalagin, punicalan, urolithin a, rosuvastatin, selenium, theaflavin-3,3′-digallate, dexamethasone, boceprevir, paritaprevir, cryptoquindoline, losartan, myricitrin, amiodarone, simeprevir, berberine, chrysin, digoxin, atazanavir, azvudine, ro-0622, azd7442, tixagevimab, cilgavimab, azithromycin, carvedilol, cetirizine, ct-p59, disulfiram, darolutamide, doxycycline, fostamatinib, masitinib, naproxen, neem, azadirachta indica a. juss, regdanvimab, ruxolitinib, saline, selamectin, silmitasertib, suramin, tannic acid, tenofovir disoproxil, valproic acid, amentoflavone, pristimerin, ribavirin, indinavir, saquinavir, cryptospirolepine, rhoifolin, pectolinarin, tamoxifen, 6-gingerol, theaflavin, oleuropein, etoposide, genistein, ibuprofen, paclitaxel, sertraline, tacrolimus, puerarin, limonin, obacunone, amprenavir, velpatasvir, ursolic acid, daidzein, sesamin, astragalin, gilteritinib, baicalein, caffeic acid, amitriptyline, apalutamide, apixaban, aframomum melegueta, aprotinin, ashwagandha, withania somnifera, bedaquiline, bergamottin, boswellic acids, calpeptin, canakinumab, chlorpheniramine, chlorphenamine, chlorhexidine, desloratadine, clarinex, diphenhydramine, doramectin, doxazosin, Echinacea purpurea, enzalutamide, forsythoside a, glycine, griffithsin, hydroxyurea, huashibaidu, jing si herbal tea, jinhua qinggan, jt001 (vv116), lenzilumab, lianhua qingwen, nelfinavir, nog19, panobinostat, paroxetine, phosphodiesterase enzyme type 5 inhibitors, pleurotus ostreatus, rheum palmatum, s-217622, tafenoquine, tofacitinib, virgin coconut oil, xuanfeibaidu, zinc pyrithione, berbamine, moxidectin, thymoquinone, xanthoangelol e, tingenone, iguesterin, micafungin, ombitasvir, tipranavir, glecaprevir, emetine, colistin, ebselen, lonafarnib, glycyrrhizin, crocin, cryptomisrine, biscryptolepine, herbacetin, cycloeucalenol, campesterol, cyanidin-3-o-glucoside, afatinib, nilotinib, scedapin c, quinadoline b, decitabine, gemcitabine, afzelin, rhein, artesunate, bufalin, camptothecin, capsaicin, chlorpromazine, cyclophosphamide, dasatinib, doxorubicin, flutamide, folic acid, hydralazine, isotretinoin, leflunomide, morin, naringenin, phenytoin, propylthiouracil, pterostilbene, reserpine, simvastatin, sirolimus, sorafenib, sulindac, tretinoin, verapamil, vincristine, bicuculline, ledipasvir, tideglusib, bemcentinib, ergotamine, taraxerol, diosbulbinoside d, estrone-2,3-quinone, hinokiflavone, ginkgetin, norquinadoline a, natamycin, raltegravir, dolutegravir, cyanidin 3-o-rutinoside, quercetin 3-o-rutinoside, magnolol, phillyrin, conivaptan, aloesin, gingerol, tinocordiside, sesaminol, sesamolin, ephedrine, solanine, donepezil, vby-825, omipalisib, psoralidin, cryptotanshinone, tetrandrine, fangchinoline, z-fa-fmk, imatinib, duloxetine, allicin, ezetimibe, pravastatin, ramipril, abemaciclib, dihydromyricetin, scutellarein, corilagin, ouabain, isorhamnetin, nystatin, sunitinib, anakinra, eculizumab, dieckol, sinigrin, glabridin, gimsilumab, kin1901, infliximab, leronlimab, pa14, pro-140, vyrologix, tocilizumab, procyanidin, α-hederin, ferulic acid, cyanidin, cyanidin 5-o-β-d-glucoside, cyanidin 3-o-glucoside, chlorogenic acid, gallic acid, λ-carrageenan, 4′-fluorouridine, 76clabs, 8g3, α6-001, agp-14, agp-15, amodiaquine, acarbose, ard-61, arq ajīb, arylazothiazolimines, aviptadil, ayurcov, beauvericin, bifonazole, bismuth subsalicylate, bis-thiadiazoles, blue light, brazilin, breathing exercises, brii-196/brii-198, bromelain, bucillamine, c60 fullerene, Camellia sinensis, cd24fc, chlorine dioxide, chyawanprash, clevudine, clitoria ternatea, asian pigeonwings, bluebellvine, blue pea, butterfly pea, cordofan pea, darwin pea, clofoctol, codivir, copper(ii) gluconate, clsp-2, ctb-ace2 gum, cysteamine, d-α-tocopherol polyethylene glycol succinate, tpgs, dfo, diammonium glycyrrhizinate, dimethyl sulfoxide, darunavir ethanolate, ebastine, ek1c4, engineered ace2, enoxaparin, epicatechin, fbr-002, estrogen, ethoxzolamide, ethyl lauroyl arginine, exo-cd24, evusheld, tixagevimab, cilgavimab, ferrocene derivatives, flovid-20, gancao-banxia, gb-1, green tea, hanshi zufei, heparin, hinokitiol, homo-harringtonine, honey, hydroxyzine, inm005, interferon-X (interferon lamba), isoprinosine, js016, kabasura kudineer, kalmegh, kinetin, led spirulina, levamisole, levilimab, 1-glutamine, ly2835219, manidipine, manuka honey, maoto, mesencure, metformin glycinate, mi-1851, montelukast, multimeric soluble ace2, n-acetylglucosamine, natto extract, naphthoquine, niacinamide, nicotinamide mononucleotide, opaganib, oseltamivir, oxygen-ozone immunoceutical therapy, palmitoylated ace2, pamapimod, pegylated interferon alpha-2b, peg ifn-a2b, pentosan polysulfate, pioglitazone, phoxwell, phthalocyanine, porphyridium sp., pranayama, probenecid, propolis sulabiroin-a, pyramax, pyronaridine-artesunate, pyrimidine, pyronaridine, q34, quinine, raloxifene, rd-x19, rejuveinix, riboflavin, sabizabulin, saliravira, sarbd-1, sars-block, schaftoside, sea cucumber sulfated polysaccharide, scsp, seraph 100 microbind affinity filter, serratiopeptidase, serratia e-15 protease, serralysin, serratiaprotease, serrapeptase, shallot, si-f019, silibinin, sng001, spirulina, sodium bicarbonate, stenoparib, sti-9167, thymic peptides, tollovir, toremifene, tpnt1, tranilast, trisb92, Urtica dioica agglutinin, zinv03977803, znonps, znsec-humicin, z-veid-fmk, minocycline, coclobine, dieckol 1, celastrol, 3-isotheaflavin-3 gallate, dihydrotanshinone i, citriquinochroman, holyrine b, proximicin c, pityriacitrin b, anthrabenzoxocinone, penimethavone a, maraviroc, valrubicin, icatibant, bepotastine, epirubicin, epoprostenol, vapreotide, aprepitant, caspofungin, perphenazine, hrsace2, tegobuvir, olysio, filibuvir, alisporivir, allyl disulphide, allyl trisulphide, digitoxigenin, tenufolin, pavetannin ci, lianhuaqingwen, dorzolamide, deptropine, neostigmine bromide, ethotoin, hydrocotarnine, ampyrone, withanolide b, lurasidone, lumacaftor, perfenazine, benserazide, isocarboxazide, cryptophycin 1, cryptophycin 52, deoxycylindrospermopsin, anatibant, pilaralisib, zabofoxacin, tiracizine, picotamide, cilazapril, indisulam, ziprasidone, propadimine, phenformin, torososide b, covitris2020, chlovid2020, silybin, licoleafol, mitomycin c, gardenin a, 6-paradol, psiadia punctulata, tanshinone-i, mangiferin, γ-mangostin, 3,3′-diindolylmethane, 4-hydroxy-2-nonenal, acetaldehyde, aflatoxin-b1, aica-ribonucleotide, alitretinoin, alpha-tocopherol, azacitidine, azathioprine, belinostat, benazepril, benzopyrene, bezafibrate, bisphenol-a, bortezomib, bucladesine, buspirone, buthionine-sulfoximine, cadmium-chloride, caffeine, carbamazepine, carbon-tetrachloride, celecoxib, ciglitazone, cisplatin, clodronic-acid, clofibrate, clozapine, colforsin, corticosterone, coumestrol, cycloheximide, cytarabine, dactinomycin, daunorubicin, deguelin, dichloroacetic-acid, diclofenac, dieldrin, diethylstilbestrol, dimethylnitrosamine, dinoprost, entinostat, estriol, ethinyl-estradiol, fenretinide, fluorouracil, fulvestrant, fumonisin-b1, furan, gefitinib, glafenine, glucosamine, haloperidol, ifosfamide, indole-3-carbinol, ionomycin, irinotecan, levofloxacin, lithium-chloride, mercaptopurine, methapyrilene, methimazole, methoxychlor, mevalonic acid, mifepristone, nimesulide, norepinephrine, ochratoxin-a, olanzapine, omeprazole, orphenadrine, oxidopamine, pentachlorophenol, phenethyl-isothiocyanate, pifithrin, pilocarpine, pirinixic acid, piroxicam, progesterone, pyrazolanthrone, ranitidine, rimonabant, rosiglitazone, rotenone, sulfasalazine, tamibarotene, testosterone, tetrachloroethylene, tetracycline, thapsigargin, theophylline, topotecan, tributyltin, trichloroethylene, trichostatin-a, triclosan, troglitazone, tunicamycin, tyrphostin-ag-1478, urethane, valdecoxib, vancomycin, vorinostat, wortmannin, zearalenone, zidovudine, zinc32960814, zinc12006217, zinc03231196, nigricanoside a, nigricanoside b, callophysin a, gallocatechin, hyperin, lupinifolin, viomycin, capastat, demethoxycurcumin, bisdemethoxycurcumin, scutellarin, alloyohimbine gummadiol, asparagamine a, vincapusine, oxytetracycline, naringin, kanamycin, cefpiramide, salvianolic acid b, teniposide, benzoylgedunin, 6-deaminosinefungin, unii-o9h5ky11sv, cephalosporin derivatives, neomycin, tcm5280805, tcm5280445, tcm5280343, tcm5280863, tcm5458190, quinadoline, polyketide isochaetochromin d1, 0-hydroxyusambarensine, 6-oxoisoiguesterin, 22-hydroxyhopan3-one, grazopavir, mfcd00832476, mfcd02180753, pacritinib, a3659, a3777, leucopelargonidin, taxifolin, eriodictyol, enterodiol, naringen, flemiflavanone d, euchrestaflavanone a, triamterene, estrone, azadirachtin-h, azadirachtin-i, azadirachtin-q, sn00334175, sn00162745, alphaspinasterol, gycyrrhizin, azadirachtani, mycophenolic acid, kushenol-w, 6-azauridine, cyanidin 5-o-β-d-glucoside, morelloflavone, methylochnaflavon, isoginkget, sciadopitysin, podocarpusflavone a, cryptomerin, cytochalasin z8, leucal, zanamivir, penciclovir, etravirine, p-coumaric acid, ketazolam, methylnaltrexone, ethynodiol diacetate, petunidin 3,5-odiglucoside, delphinidin 3-o-rutinoside, grazoprevir, las 51620435, las 51620429, rosoxacin, levomefolic, etodolac, tenofovir, tinofoviralafenamide, ornipressin, otosiban, lanreotide, argiprestocin, demoxytocin, carbetocin, lypressin, examorelin, polymyxin b1, verbascoside, abrisapogenol g, kaempferol-3-o-rutinoside, hederagenin, abrusoside a, robustaflavone, agathisflavone, swertianolin, ononin, pedunculoside, imperialine, peimisine, batatasin i, pomiferin, coumarin, rhodionin, aloin, sarsasapogenin, silydianin, rutaecarpine, morusin, α-mangostin, yohimibine, coclaurine, strychnine, myristicin, medicarpin, coptisine, tiliroside, glabrone, lignans, gastrodin, cordycepin, ajugol, evodiamine, nuciferine, cynarin, betulin, gramine, narciclasine, vitexin, protopanaxatriol, karanjin, gigantol, harsingar, nictoflorin, lupeol, aloevera, aloenin, giloy, sitosterol, nimbin, ginger, shogaol, cyanin, medicagol, faradiol, flavanthrin, withanoside v, somniferine, vicenin, isorientin, sesamolinol, ellipticine, ecteinascidin, homoharringtonine, dolastatin, halichondrin, plicamycin, arvoside, nicotiflorin, kaempferol sulphates, quercetin-3-o-glucuronide, isorhamnetin-3-o-glucoside, narcissin, calendula glycoside b, lauruside, miricetine-3-glucoside, cordifoliside, anthraquinones, emodine 8-glucosides, acetoside, lisinopril, deoxynortryptoquivaline, thalimonine, sophaline, tomatidine, 10-hydroxyusambarensine, stychnopentamine, usambarensine, vanillin, crambescidin 786, crambescidin 826, sepiapterin, tetrodotoxin, caulerpin, lutein, galanthamine, nomilin, deacetylnomilin, ichangin, amyrin, 24-dimethylene cycloartenol, isoiguesterin, lentinula edodes, agaricus bisporus, jmx0286, jmx0301, jmx0941, erythromycin, chloramphenicol, mpi43, mpi44, mpi46, keigairengyoto, shosaikotokakikyosekko, kakkontokasenkyushin'i, vuwcov059, vwcov267, vuwcov270, 4-hydroxycordoin, mallotophilippen d, 3′-(3-methyl-2-butenyl)-4′-o-β-d-glucopyranosyl-4,2′-dihydroxychalcone, lefamulin, cefixime, cefpodoxime, ciprofloxacin, sparfloxacin, moxifloxacin, tbaj-876, (s)-crizotinib, spermidine, spermine, mk-2206, saffron, mdl-28170, z lvg chn2, ono 5334, mln-3897, tipifarnib, salinomycin, bavachin, tanshinone iia, isobavachalcone, lycorine hydrochloride, lycorine, bufotaline, cinobufagin, periplocoside, veratridine, coniferyl aldehyde, cornuside, brusatol, momordinic, roburicacid, hruceine a, isoalantolactone, oridonin, dehydrocostus lactone, alantolactone, dehydrodiisoeugenol, liensinine, isoliensinine, vps34-in1, stf-62247, mcoppb, gw 803430, amodiaquine dihydrochloride, n-methylspiperone, clemastine fumarate, gmc 2-29, lu ae58054, chlorprothixene, methdilazine, methotrimeprazine maleate, piperacetazine, difeterol, loperamide, naltrindole isothiocyanate, am1241, cpdd, sb 271046, gmc 2-113, caa-0225, cathepsin inhibitor 1, z-gly-leu-phechloromethyl ketone, balicatib, calpain inhibitor i, berzosertib, ikk-2 inhibitor viii, nse 33994, alpha-l-arabinopyranose, m1414, it1t dihydrochloride, s-15176 difumarate, jtv519 hemifumarate, rescimetol, trifluomeprazine 2-butenedioate, asteriscunolide d, genz-123346, maprotiline, deserpidine, melitracen, dlunarizine, proglumetacin, dmp 777, dexanabinol, trifluoperazine 2hcl, thioridazine hcl, bicalutamide, cinnamaldehyde, piperine, zingeberene, gr 127935, diazepam, levetiracetam, sotalol, tolterodine, oxazepam, thiamazole, hydroxycarbamide, clonazepam, meloxicam, temazepam, anastrozole, rivaroxaban, perindopril, silodosin, lorazepam, pramipexole, bupropion, venlafaxine, bisoprolol, aripiprazole, linagliptin, clopidogrel, allopurinol, indapamide, chlortalidone, nifedipine, warfarin, phylloflavan, milk thistle, ilexin b, isosilybin b, spiperone, vitamin b12, vitamin b9, vitamin k1, vitamin k2, vitamin e, oleanderolide, proceragenin a, balsaminone a, anacardic acid, aloeresin d, tcid, dihydrokaempferol, dihydroquercetin, myricentin, isoquercitrin, pc000550, pc000361, pc000558, pc000573, gr10617, mefloquine, brequinar, dipyridamole, quercetagetin, glycycrrhizin, δ9-tetrahydrocannabinol, arteether, dihydroartemisinin, arteannuin, cannabidinol, punicalin, panduratin a, andrographis paniculata, Zingiber officinale, Boesenbergia rotunda, scutellaria baicalensis, pomegranate peel extract, zinc000013444414, zinc000137976768, zinc000143375720, diosmin, apiin, okadaic acid, p-57as3, concanamycin a, amphotericin b, oleandrin, gitoformate, eprinomectin, beta-escin, fusicoccin, perilla aldehyde, perillyl alcohol, cidofovir, valaciclovir, tazarotene, hydroquinone, bromocriptine, acyclovir, finasteride, betamethasone, clonidine, somatotropin, nitisinone, tadalafil, vinblastine, pyrimethamine, pentamidine, etacrynic acid, proguanil, everolimus, tamsulosin, rifapentine, terbinafine, fluvastatin, cyclosporine, hydrocortisone, pentoxifylline, nitrogen, arginine, abatacept, interferon beta-1a, thalidomide, crizanlizumab, nitroglycerin, fluorescein, captopril, brexanolone, omalizumab, siltuximab, propranolol, interferon beta-1b, prednisone, prednisolone, methylprednisolone, torin-2, rapamycin, radotinib, thiostrepton, c1-amidine, bb-c1-amidine, broussoflavan a, hygromycin b, nabiximols, abyssinone ii, procynidin b1, indigo blue, crytospirolepine, 10′-hydroxyusambaresine, strychnopentamine, usararotenoid a, 12α-epi-milletosin, torchnil, febcin, pibrentasvir, 5-chloro-omega-hydroxy-1-o-methylemodin, cystodion e, jq1, zbc260, zafirlukast, pranlukast, canderastan cilexetil, saquanivir, boron citrate, oleoylethanolamide, liquiritin, laminarin, eckol, trifucol, β-d-galactose, κ-carrageenan, c135-1s/c144-1s, sab-185, vir-7831/vir-7832, covi-amg/covi-drops, covi-guard, 2-deoxy-d-glucose, dn1758, lanadelumab, abivertinib, bld-2660, pemziviptadil, artesunate/pyronaridine, carragelose, at-527, ptc299, brilacidin, saracatinib, erlotinib, osimertinib, pictilisib, dinaciclib, cigb-325, sb203580, ralimetinib, mapk13-in-1, arry-797, tofacitinibfedratinib, mpro 13b, gc-373, gc-376, mpro n3, bosutinib, clofazimine, domperidone, entecavir, fedratinib, ipratropium bromide, lomitapide, metoclopramide, s1ra, thioguanine, sitagliptin, dihydroergotamine, crinine, ilexsaponin b2, strictinin, zinc000027215482, zinc000252515584, loniflavone, 2′-o-ribose methyltransferase, tirucallin a, zinc000253504770, zinc000253504766, triamcinolone, amoxicillin, hydrochlorothiazide, terflavin a, chebulagic acid, chebulinic acid, coumaroylquinic acids, sinapoyl d glucoside, tetra-o-galloyl-β-d-glucose, methyl rosmarinates, cosmosiin, dihydronitidine, lectin, digitoxigenine, calarene, amaranthin, diarylheptanoids, indigo, aloe emodin, dihydrocelastrol, phyllaemblinol, isocolumbin, magnoflorine, piperolactam a, withanone, biflavone, cinnamic amides, agomelatine, ramelteon, immunoglobulin, ifn-β1a, ifn-β1b, interleukin-2, cynk-001, baloxavir marboxil, asc09, danoprevir, cobicistat, carrimycin, dihydroartemisinine, piperaquine, fingolimod, piclidenoson, cf101, nivolumab, obtivo, meplazumab, jakotinib, tj003234, tozumab, adamumab, ravulizumab, alxn1210, clazakizumab, avdoralimab, iph5401, ly3127804, ifx-1, bevacizumab, valsartan, 7-hydroxystaurosporine, bafetinib, emtricitabine, adefovir, tenofovir alafenamide, abacavir, ganciclovir, didanosine, delavirdine, pirfenidone, β-glucan, p-coumaroyltriacetic acid lactone, zinc02111387, zinc02122196, sn00074072, zinc04090608, xuebijing, lung cleansing and detoxifying decoction, etanercept, ibrutinib, enalapril, telmisartan, oroxylin a, irisolidone, anhydrosafflor yellow b, euchrenone, uncaric acid, demethylzeylasteral, maslinic acid, atractylenolide iii, astragaloside iv, daturaolone, cucurbitacin g 2-glucoside, citronellol, limonene, salvianolic acid, neochlorogenic acid, kobophenol a, bis-demethoxycurcumin, 6-viniferin, pseudoephedrine, methylephedrine, speciophylline, uncarine f, anisodamine, forsythoside i, amygdalin, urso-deoxycholic acid, withanolide a, β-sitosterol, farnesiferol b, amlodipine, dexchlorpheniramine, tanshinones, isatis indigotica root, rhizoma cibotii, torreya nucifera, shuang huang lian, estragole, eugenol, ubiquinone, flecainide, poly-methoxyflavones, poly-hydroxyflavones, mearnsitrin, 3-o-β-d-glucoside, benzoic acid, biorobin, delphinidin, galangin, isoferulic acid, dithymoquinone, negillicine, negillidine, muc1, α-lactalbumin, or mixtures thereof.

In another embodiment, provided are the method when the non-covalent complex, molecular/solid dispersion or conjugate is combined with at least one additional active pharmaceutical ingredient (AAPI) active (in vitro, in vivo, ex vivo, in silico) against disease (non limiting example: cancer, etc) and selected from: 5-Fluorouracil; Abarelix; Abiraterone; Acetaminophen; Acetaminophen or paracetamol; Acetazolamide; Actiq; Adcretris; Adriamycin RDF; Afinitor; Agomelatine; Agrylin; Albendazole; Aldara; Aldesleukin; Alemtuzumab; Alfuzosin; Alimta; Aliskiren; Alitretinoin; Alkeran; All-trans retinoic acid or tretinoin; Allopurinol; Aloxi; Alpha-Lipoic Acid; Altretamine; Amantadine; Amifostine; Amiloride; Aminoglutethimide; Aminolevulinic Acid; Amiodarone; Amitriptyline; Amlodipine; Amodiaquine; Amsacrine; Anagrelide; Anakinra; Anastrozole; Anexsia; Anzemet; Aprepitant; Aprotinin; Arava; Aredia; Arginine; Arimidex; Aripiprazole; Aromasin; Arranon; Arsenic Trioxide; Artesunate; Arzerra; Asparaginase Erwinia Chrysanthemi; Aspirin; Atenolol; Atorvastatin; Atovaquone; Atrial Natriuretic Peptide; Auranofin; Avastin; Axitinib; Azathioprine; Azithromycin; Bazedoxifene; Bedaquiline; Bemiparin; Bendamustine; Benserazide; Bepridil; Bevacizumab; Bexarotene; Bexxar; Bezafibrate; BiCNU; Bicalutamide; Biperiden; Blenoxane; Bleomycin; Bortezomib; Bosentan; Bosulif; Bosutinib; Brentuximab Vedotin; Bromfenac; Bromocriptine; Busulfan; Cabazitaxel; Cabergoline; Cabozantinib; Caffeine; Calcitriol; Campath; Camptosar; Candesartan; Capecitabine; Captopril; Carbimazole; Carboplatin; Carfilzomib; Carglumic acid; Carmustine; Carvedilol; Casodex; CeeNU; Celebrex; Celecoxib; Cephalexin; Cerubidine; Cetuximab; Chlorambucil; Chloroquine; Chlorpromazine; Cholecalciferol; Cidofovir; Cilnidipine; Cimetidine; Cinacalcet; Ciprofloxacine; Cisplatin; Citalopram; Cladribine; Clarithromycin; Clofarabine; Clofoctol; Clolar; Clomifene; Clomipramine; Clotrimazole; Colchicine; Cometriq; Conjugated Estrogens; Cosmegen; Crizotinib; Cyclophosphamide; Cytabarine; Cytadren; Cytarabine; Cytosar-U; Cytoxan; DES; DTIC-Dome; Dacarbazine; Dactinomycin; Dalteparin; Danazol; Dapsone; Dasatinib; Daunorubicin; Deferoxamine; Degarelix; Denosumab; Desmopressin; Diclofenac; Diethylstilbestrol; Diflunisal; Digitoxin; Digoxin; Dimethyl Fumarate; Dipyridamole; Disulfiram; Docetaxel; Dolasetron; Donepezil; Doxazosin; Doxorubicin; Doxycycline; Ebastine; Efavirenz; Eflornithine; Elitek; Ellence; Eloxatin; Emcyt; Emend; Enalapril; Enoxaparin; Enzalutamide; Epalrestat; Epirubicin; Erbitux; Eribulin; Erivedge; Erlotinib; Erwinaze; Esomeprazole; Estramustine; Ethacrynic acid; Ethyol; Etodolac; Etoposide; Eulexin; Everolimus; Evista; Exemestane; FOLFIRI; FOLFIRI-Bevacizumab; FOLFIRI-Cetuximab; FOLFIRINOX; FOLFOX; Famotidine; Fareston; Faslodex; Fasudil; Felodipine; Femara; Fenofibrate; Fentanyl; Fingolimob; Fish oil (EPA/DHA); Flubendazole; Fludara; Fludarabine; Fluorouracil; Fluoxetine; Fluspirilene; Flutamide; Fluvastatin; Fluvoxamine; Folinic Acid; Folotyn; Fulvestrant; Fusilev; Gardasil; Gefitinib; Gemcitabine; Gemtuzumab Ozogamicin; Gemzar; Gleevec; Glipizide; Glucarpidase; Glutamine; Goserelin; Granisetron; Griseofulvin; Halaven; Haloperidol; Herceptin; Hexalen; Hycamtin; Hydralazine; Hydrocodone; Hydroxychloroquine; Hymecromone; Ibandronate; Ibritumomab Tiuxetan; Ibuprofen; Iclusig; Idamycin; Idarubicin; Ifex; Ifosfamide; Imatinib; Imipramine; Imiquimod; Imuran; Indomethacin; Ingenol Mebutate; Inlyta; Interferon Alfa-2b; Intron A; Iodixanol; Ipilimumab; Irbesartan; Iressa; Irinotecan; Irinotecan); Istodax; Itraconazole; Ixabepilone; Ixempra; Jakafi; Jevtana; Kadian; Kepivance; Ketoconazole; Ketorolac; Kyprolis; Kytril; Lanreotide; Lansoprazole; Lapatinib; Leflunomide; Letrozole; Leucovorin; Leukeran; Leukine; Leuprolide; Leustatin; Levetiracetam; Levofloxacin; Levulan; Licofelone; Lidocaine; Lithium; Lomustine; Loperamide; Loratadine; Losartan; Lovastatin; Loxoprofen; Lupon Depot; Lysodren; Macitentan; Manidipine; Maraviroc; Masoprocol; Matulane; Mebendazole; Mechlorethamine; Mechloroethamine; Meclofenamate; Mefloquine; Megace; Megestrol; Megestrol acetate; Melatonin; Meloxicam; Melphalan; Memantine; Mepacrine or Quinacrine; Mercaptopurine; Mesna; Metformin; Methazolamide; Methimazole; Methotrexate; Methoxsalen; Methyl Aminolevulinate; Methylnaltrexone; Methylprednisolone; Metoclopramide; Metvixia; Mifepristone; Minocycline; Mirtazapine; Mitomycin; Mitotane; Mitoxantrone; Montelukast; Morphine; Mozobil; Mutamycin; Mycophenolate; Mycophenolic Acid; Myfortic; Myleran; Mylotarg; Nadroparin; Naltrexone; Naproxen; Navelbine; Nelarabine; Nelfinavir; Neulasta; Neumega; Neutroval; Nexavar; Niclosamide; Nifedipine; Nifurtimox; Nilandron; Nilotinib; Nilutamide; Nimodipine; Nipent; Nisodilpine; Nitazoxanide; Nitisinone; Nitroglycerine; Nitroxoline; Nolvadex; Norethandrolone; Noscapine; Novantrone; Nplate; Octreotide; Ofatumumab; Olanzapine; Olsalazine; Omacetaxine; Omeprazole; Oncaspar; Oncovin; Ondansetron; Oprelvekin; Orlistat; Ormeloxifene; Oseltamivir; Ouabain; Oxaliplatin; Oxcarbazepine; Paclitaxel; Palifermin; Palonosetron; Pamidronate; Panitumumab; Pantoprazole; Paraplatin; Pazopanib; Pegaspargase; Pegfilgrastim; Peginterferon Alfa-2b; Pemetrexed; Penfluridol; Pentamidine; Pentostatin; Pentoxifylline; Perjeta; Perphenazine; Pertuzumab; Phentolamine; Phenylbutyrate; Phenytoin; Photofrin; Picato; Pimozide; Pioglitazone; Pipobroman; Pirfenidone; Platinol; Plenaxis; Plerixafor; Ponatinib; Porfimer; Pralatrexate; Pravastatin; Prazosin; Prednisone; Pregabalin; Premarin; Procarbazine; Progesterone; Prograf; Proleukin; Promethazine; Prometrium; Propranolol; Provenge; Purinethol; Pyrimethamine; Pyrvinium pamoate; Quadramet; Quadrivalent Human Papillomavirus Vaccine; Quetiapine; Rabeprazole; Raloxifene; Raltitrexed; Ranolazine; Rapamune; Rasburicase; Regorafenib; Repaglinide; Ribavirin; Rifabutin; Riluzole; Risperidone; Ritonavir; Rituxan; Rituximab; Roflumilast; Romidepsin; Romiplostim; Rosuvastatin; Ruxolitinib; Samarium Sm 153 Lexidronam; Sargramostim; SecreFlo; Secretin; Sensipar; Sertraline; Sildenafil; Simvastatin; Sipuleucel-T; Sirolimus; Sodium Bicarbonate; Sodium Oxybate; Sorafenib; Spironolactone; Sprycel; Stivarga; Streptozocin; Sulfasalazine; Sulindac; Sunitinib; Sutent; Sylatron; Synribo; Tacrolimus; Tadalafil; Tamoxifen; Tarceva; Targretin; Tasigna; Taxol; Taxotere; Telmisartan; Temodar; Temozolomide; Temsirolimus; Teniposide; Terbinafine; Tetrathiomolybdate; Thalidomide; Thalomid; Thiabendazole; Thioguanine; Thioridazine; Ticagrelor; Ticlopidine; Tigecycline; Timolol; Tinzaparin; Tolfenamic acid; Tomudex; Topiramate; Topotecan; Toremifene; Torisel; Tositumomab; Tranexamic acid; Trastuzumab; Trazodone; Treanda; Trelstar Depot; Triamterene; Trifluoperazine; Triptorelin; Trisenox; Tykerb; Ulinastatin; UroXatral; Urokinase; Uvadex; Valproic acid; Valrubicin; Valsartan; Valstar; Vandetanib; VePesid; Vectibix; Velban; Velcade; Vemurafenib; Verapamil; Vercyte; Vinblastine; Vincristine; Vindesine; Vinorelbine; Visipaque; Vismodegib; Voraxaze; Vorinostat; Votrient; Vumon; Warfarin; Xalkori; Xeloda; Xgeva; Xibrom; Xtandi; Yervoy; Zanosar; Zelboraf; Zevalin; Zofran; Zoladex; Zoledronate; Zoledronic Acid; Zolinza; Zometa; Zytiga; tbo-Filgrastim,

and from:
(+)-Camptothecin (VSJKWCGYPAHWDS-UHFFFAOYSA-N); (+/−)-10-Hydroxycamptothecin (HAWSQZCWOQZXHI-UHFFFAOYSA-N); (−)-Bilobalide from ginkgo leaves (MOLPUWBMSBJXER-ISSLQHLCSA-N); (−)-Epicatechin (PFTAWBLQPZVEMU-UKRRQHHQSA-N); (−)-Parthenolide (KTEXNACQROZXEV-PWNZVWSESA-N); (−)-trans-Dihydronarciclasine (SBTGHBALOCEVOR-ZZTUZTEWSA-N); (1-Thio-beta-D-glucopyranosato)(triethylphosphine)gold 2,3,4,6-tetraacetate (AUJRCFUBUPVWSZ-UHFFFAOYSA-M); (10-Methylanthracen-9-yl)methyl carbamimidothioate hydrochloride (VIBMUYOXJUCEMA-UHFFFAOYSA-N); (1E)-1-Pyridin-2-ylethanone quinolin-2-ylhydrazone (ZHXBMYNQHKXYCJ-XDHOZWIPSA-N); (1E,4E)-1,5-Bis(2-methoxyphenyl)penta-1,4-dien-3-one (RCZMPCUUTSDNAJ-PHEQNACWSA-N); (1S,4aS,11S,11aS,12aS)-3-carbamoyl-1-(dimethylazaniumyl)-4a,5,7,11-tetrahydroxy-11-methyl-4,6-dioxo-1,4,4a,6,11,11a,12,12a-octahydrotetracen-2-olate (NWXMGUDVXFXRIG-WESIUVDSSA-N); (1S,4aS,11aR,12aS)-3-carbamoyl-10-(dimethylamino)-1-(dimethylazaniumyl)-4a,5,7-trihydroxy-4,6-dioxo-1,4,4a,6,11,11a,12,12a-octahydrotetracen-2-olate (FFTVPQUHLQBXQZ-KVUCHLLUSA-N); (1Z,4E)-1,5-bis(5-bromo-2-hydroxyphenyl)penta-1,4-dien-3-one (GHQJYKKGXCSWOF-SOSXVSKCSA-N); (2″Z,3″E)-5-Nitro-5″-hydroxy-indirubin-3″-oxime (KDYHYRKDQJLNGS-UHFFFAOYSA-N); (2,4-Dichlorophenoxy)tributylstannane (FFQFQPHOBAMGEV-UHFFFAOYSA-M); (2-Azanidylcyclohexyl)azanide; tetrachloroplatinum(2+) (KVFQUEDZECDHKS-UHFFFAOYSA-J); (2-Chlorophenyl) 2-[[2-(trifluoromethyl)pyridin-4-yl]amino]benzoate (OISCLGVVNMPCQX-UHFFFAOYSA-N); (2-Methoxyquinolin-4-yl)-(4-phenylpiperazin-1-yl)methanone (KUMVNHAVDWQQBN-UHFFFAOYSA-N); (2-Oxo-3-triphenylphosphaniumylpropyl)-triphenylphosphanium; chloride ion (JPBXNTVCCVWHCZ-UHFFFAOYSA-M) salts; (2R,3R,4R,5S)-1-butyl-2-(hydroxymethyl)piperidine-3,4,5-triol; hydron; chloride (QPAFAUYWVZMWPR-ZSOUGHPYSA-N); (2Z)-2-[(1-methyl-5-nitroimidazol-2-yl)methylidene]-1-benzofuran-3-one (WBYWEHLFFOFMMD-POHAHGRESA-N); (2Z)-2-[(5-nitrothiophen-2-yl)methylidene]-1-benzofuran-3-one (FFONSOSBDNEBEX-XFFZJAGNSA-N); (2Z)-5-bromo-2-[(5-nitrothiophen-2-yl)methylidene]-1-benzofuran-3-one (ADNMHZKFYBUXDA-WDZFZDKYSA-N); (2Z)-5-chloro-2-[(5-nitrothiophen-2-yl)methylidene]-1-benzofuran-3-one (QAMHJGDDFCFPGL-WDZFZDKYSA-N); (2Z)-5-methoxy-2-[(5-nitrothiophen-2-yl)methylidene]-1-benzofuran-3-one (FHHMWWQVMIUJDG-GHXNOFRVSA-N); (2Z,4E)-2-(benzenesulfinyl)-5-(3-chlorophenyl)penta-2,4-dienenitrile (WCMYWACCHAFCPJ-UWNNVHOVSA-N); (2Z,4E)-2-(benzenesulfinyl)-5-phenylpenta-2,4-dienenitrile (LKFCYVJDWGLXGU-DESBJYGPSA-N); (3-Methoxyphenyl)(oxo)arsane (VHYPDQBADMLRDN-UHFFFAOYSA-N); (3E,5E)-3,5-Bis((2-fluorophenyl)methylene)-4-piperidinone (NIVYQYSNRUIFIF-KAVGSWPWSA-N); (3E,5E)-3,5-bis[(4-nitrophenyl)methylidene]-1,1-dioxothian-4-one (SSAMIOWVUGBIJK-VULZFCBJSA-N); (3S)-1-cyano-N-(5-phenyl-1,3-thiazol-2-yl)pyrrolidine-3-carboxamide (DPOQOSJMDTZFOR-LBPRGKRZSA-N); (3S)-1-cyano-N-(5-pyridin-4-yl-1,3-thiazol-2-yl)pyrrolidine-3-carboxamide (PWOVGFSZWNUABM-NSHDSACASA-N); (3S,4E,15Z)-4,15-Docosadiene-1-yne-3-ol (HEXQBSKJVUFXNG-JYHGMTFVSA-N); (3Z,5E)-3,5-bis[(4-bromophenyl)methylidene]-1-propanoylpiperidin-4-one (DFBZUNQYQDFHPA-MJZABRMRSA-N); (3|A)-cholest-5-en-3-ylpropanedioic acid (JIEJBESLFFLEPM-TVABIKMWSA-N); (4-Methoxyphenyl)[4-(oxoarsanyl)phenyl]methanone (NFADCVNKOTVVFE-UHFFFAOYSA-N); (4S)-N-dodecyl-2-(3-fluoro-4-methoxyphenyl)-1,3-thiazolidine-4-carboxamide (ZVUZFRNDWCIFLX-PPUHSXQSSA-N); (5,10,11-Trimethylpyrido[3,4-b]carbazol-7-yl)methanol (XTLGSAQUBLLZQB-UHFFFAOYSA-N); (5,6,11-Trimethylpyrido[4,3-b]carbazol-9-yl)methanol (BLYVGTXMLDIHMT-UHFFFAOYSA-N); (5,7-dichloro-8-hydroxyquinolin-2-yl)methyl 1H-indole-2-carboxylate (DTMGDZDJTHMJDY-UHFFFAOYSA-N); (5-Ethylsulfanyl-2-phenyl-1,3-oxazol-4-yl)-triphenylphosphanium; iodide (RJUYAAJGFQIVQL-UHFFFAOYSA-M); (5R,6S)-6-Phenyl-5-[4-(2-pyrrolidin-1-ium-1-ylethoxy)phenyl]-5,6,7,8-tetrahydronaphthalen-2-ol; chloride (SFWYWKVSAHDQRB-HBYDGSNJSA-N); (6aR,12bS)-5,6,6a,7,8,12b-hexahydrobenzo[a]phenanthridine-10,11-diol (BGOQGUHWXBGXJW-RHSMWYFYSA-N); (9-Ethylcarbazol-3-yl)-quinolin-5-ylmethanol (TZOSKHBHYRMLGA-UHFFFAOYSA-N); (E)-(4-(2-(1H-indazol-3-yl)vinyl)phenyl)(piperazin-1-yl)methanone (YYLKKYCXAOBSRM-JXMROGBWSA-N); (E)-3-(1H-indol-3-yl)-2-methyl-1-(3,4,5-trimethoxyphenyl)prop-2-en-1-one (MPPKERSKNOSLQM-UKTHLTGXSA-N); (E)-3-(furan-2-yl)-N-(5-(3-(trifluoromethyl)benzyl)thiazol-2-yl)acrylamide (JQHPATIDMZKJBA-VOTSOKGWSA-N); (E)-3-methoxy-6-((2,4,6-trimethoxystyrylsulfonyl)methyl)pyridin-2-amine (SUVXJIREQOOYGN-BQYQJAHWSA-N); (E)-5-chloro-2-(2-(phenyl(pyridin-2-yl)methylene)hydrazinyl)pyridine (YHHFKWKMXWRVTJ-OQKWZONESA-N); (Glycolato-O,O′)diammineplatinum(II) (KLNFSAOEKUDMFA-UHFFFAOYSA-N); (R)-[(2S,5R)-5-ethenyl-1-azabicyclo[2.2.2]octan-2-yl]-quinolin-4-ylmethanol (KMPWYEUPVWOPIM-YNRGSOABSA-N); (R,R)-asenapine (VSWBSWWIRNCQIJ-GJZGRUSLSA-N); (S)-1-(4-(1H-Pyrazol-4-yl)phenyl)-2-amino-1-(4-chlorophenyl)ethanol (IIRWNGPLJQXWFJ-KRWDZBQOSA-N); (S)-2-(2-(2-Methylindolin-1-yl)-2-oxoethyl)-6-morpholinopyrimidin-4(3H)-one (UAXHPOBBKRWJGA-ZDUSSCGKSA-N); (S)-5-(3-Fluorophenyl)-N-(piperidin-3-yl)-3-ureidothiophene-2-carboxamide (IAYGCINLNONXHY-LBPRGKRZSA-N); (S)—N-Hydroxy-4-(3-methyl-2-phenylbutanamido)benzamide (LAMIXXKAWNLXOC-INIZCTEOSA-N); (S)-[(2R,5R)-5-ethenyl-1-azabicyclo[2.2.2]octan-2-yl]-quinolin-4-ylmethanol (KMPWYEUPVWOPIM-FGVBSWQGSA-N); (S)-crizotinib (KTEIFNKAUNYNJU-LBPRGKRZSA-N); (S)9-Amino-20-O—(N,N-dimethyl)glycinyl camptothecin·HCl (VCFQXXKUPMCLFP-JIDHJSLPSA-N); (S,E)-3-(6-Bromopyridin-2-yl)-2-cyano-N-(1-phenylethyl)acrylamide (VFUAJMPDXIRPKO-LQELWAHVSA-N); (S,E)-3-(6-bromopyridin-2-yl)-2-cyano-N-(1-phenylbutyl)acrylamide (LIDOPKHSVQTSJY-VMEIHUARSA-N); (Z)-3,3′,4,4′,5-Pentamethoxystilbene (KMCRDRIVRPRGHU-SREVYHEPSA-N); (Z)-3,4,5-Trimethoxy-4′-bromostilbene (UBBBWYGKBDCYDY-PLNGDYQASA-N); (Z)-3,4,5-Trimethoxy-4′-ethoxystilbene (DSXJJVAVIOTERY-SREVYHEPSA-N); (Z)-3,4,5-Trimethoxy-4′-ethylstilbene (ATPLLJHVRJWGIN-KHPPLWFESA-N); (Z)-3,4,5-Trimethoxy-4′-methylstilbene (YSJFPRSDNPCGBF-KTKRTIGZSA-N); (Z)-4-(1-(4-(2-(Dimethylamino)ethoxy)-phenyl)-2-phenylbut-1-en-1-yl)phenol (TXUZVZSFRXZGTL-UHFFFAOYSA-N); (Z)-5-((4-(pyridin-4-yl)quinolin-6-yl)methylene)thiazolidine-2,4-dione (QDITZBLZQQZVEE-YBEGLDIGSA-N); (e)-5-(3-(4-Chlorophenyl) acryloyl)-2-methoxybenzenesulfonamide (IMNALXGNYWNIEP-XBXARRHUSA-N); (e)-Bis(3-nitrophenyl)diarsene (CKTGDBNPJIMZJP-UHFFFAOYSA-N); 1′-Epi Gemcitabine Hydrochloride (OKKDEIYWILRZIA-UHFFFAOYSA-N); 1(4H)-Naphthalenone, monohydrochloride (GBXFEIWAUUUQFE-UHFFFAOYSA-N); 1, Acanthifolicin deriv. (QNDVLZJODHBUFM-IHRYTRHCSA-N); 1,1-dimethyl-3-[(E)-1-pyridin-2-ylethylideneamino]thiourea (ZMZIYBQONAEPNV-XYOKQWHBSA-N); 1,1-dipropyl-3-[(E)-2-pyridylmethyleneamino]thiourea (ARFYQGPGBDRRSP-RVDMUPIBSA-N); 1,10-Phenanthroline (DGEZNRSVGBDHLK-UHFFFAOYSA-N); 1,10-Phenanthroline-5,6-dione (KCALAFIVPCAXJI-UHFFFAOYSA-N); 1,2,3,10-Tetrahydrocyclopenta[c][1,8]phenanthrolin-11-one (MXLQFLFNRRWURU-UHFFFAOYSA-N); 1,2-Diaminocyclohexane platinum (II) chloride (UUERTZXRKZEANK-UHFFFAOYSA-L); 1,2-Dimethoxy-4-(2-nitropropenyl)benzene (JGFBGRHDJMANRR-VURMDHGXSA-N); 1,2-Ethanediol (LYCAIKOWRPUZTN-UHFFFAOYSA-N); 1,3,5-Triazin-2(1H)-one, 4-amino-1-beta-D-ribofuranosyl-(NMUSYJAQQFHJEW-UHFFFAOYSA-N); 1,3-Bis(3,4-dichlorophenyl)urea (ZDPIZPXVHVYTOK-UHFFFAOYSA-N); 1,3-Bis(naphthalen-2-ylmethyl)-2-phenylbenzimidazol-3-ium ion (WTCALBGKYLSUTM-UHFFFAOYSA-N) salts; 1,3-Diphenylguanidine (OWRCNXZUPFZXOS-UHFFFAOYSA-N); 1,3-Propanediamine, N-hexyl-N′-(1-nitro-9-acridinyl)-(NSZWREFZGBFSJX-UHFFFAOYSA-N); 1,4-Bis((2-aminoethyl)amino)-5,8-dihydroxy-9,10-anthraquinone (CLVFWRBVFBUDQU-UHFFFAOYSA-N); 1,4-Bis(2-methylaziridin-1-yl)butane-2,3-diol (JIXGRSTZINBWQO-UHFFFAOYSA-N); 1,4-Diaminobutane (KIDHWZJUCRJVML-UHFFFAOYSA-N); 1,4-Diazabicyclo[2.2.1]heptane diperchlorate ion (INPXLFGYPJVNCP-UHFFFAOYSA-M) salts; 1,4-Dimethoxy-2-[(E)-2-nitroethenyl]benzene (IRRZIWHEPWPPJF-AATRIKPKSA-N); 1,8-Dihydroxy anthraquinon (CRHREXGMDRBOQO-UHFFFAOYSA-N); 1,8-Octanediamine, N,N′-bis(3-methoxy-9-acridinyl)-(APEGUHYDSVLTGU-UHFFFAOYSA-N); 1,9-Pyrazoloanthrone (ACPOUJIDANTYHO-UHFFFAOYSA-N); 1-((4-methoxyphenyl)amino)-2H-naphtho[1,2,3-de]quinoline-2,7(3H)-dione (YTIARIJWCIYGIC-UHFFFAOYSA-N); 1-(2-Chloro-4-(6,7-dimethoxyquinazolin-4-yloxy)phenyl)-3-propylurea (VPBYZLCHOKSGRX-UHFFFAOYSA-N); 1-(2-Chloroethyl)-1-nitroso-3-(2-norbornyl)urea (VGFFLHYAQABHTR-UHFFFAOYSA-N); 1-(2-Chloroethyl)-1-nitrosourea (NVKGVBZZSJFQLM-UHFFFAOYSA-N); 1-(2-Chloroethyl)-3-(2,6-dioxo-3-piperidyl)-1-nitrosourea (KHWIRCOLWPNBJP-UHFFFAOYSA-N); 1-(2-Ethylphenyl)-4-(3,4,5-trimethoxybenzoyl)piperazine (GTFTUVWVYOUTFW-UHFFFAOYSA-N); 1-(3-Chlorophenyl)piperazine (VHFVKMTVMIZMIK-UHFFFAOYSA-N); 1-(3-chlorophenyl)-3-[(Z)-[phenyl(pyridin-2-yl)methylidene]amino]thiourea (DDMBEZBBDVOBMQ-NKFKGCMQSA-N); 1-(4-Methoxyphenyl)-2-(3,4,5-trimethoxyphenyl)ethene (GGFQQRXTLIJXNY-WAYWQWQTSA-N); 1-(4-Methylbenzamido)-2-(chloroacetamido)-anthraquinone (LYJYMKFVNDQMAF-UHFFFAOYSA-N); 1-(4-bromo-2-chlorophenyl)-1H-pyrrole-2,5-dione (YWJRXLQKQIZCMI-UHFFFAOYSA-N); 1-(4-methoxyphenyl)-3-[(E)-[phenyl(pyridin-2-yl)methylidene]amino]thiourea (RLZRXWZKBWZLGC-FCDQGJHFSA-N); 1-(5-(2,3-Dichlorophenylthio)-4-nitrothiophen-2-yl)ethanone (LKZLGMAAKNEGCH-UHFFFAOYSA-N); 1-(5-Chloro-2-methoxyphenyl)-3-(2-methylquinolin-4-yl)urea (YBLWOZUPHDKFOT-UHFFFAOYSA-N); 1-(cyclohexylamino)-3-[4-[(E)-1,2-diphenylprop-1-enyl]phenoxy]propan-2-ol (CGDLHRTVGJPHNE-JJKYIXSRSA-N); 1-(dimethylamino)-4-methyl-4H-pyrido[3,4-g]quinoline-5,10-dione (PCSMOWAHBVADCZ-UHFFFAOYSA-N); 1-(p-Tolylsulfonyl)-3,5-bis(3,5-difluorobenzylidene)-4-piperidone (VEOCDYGHNCQFKS-UPICDLATSA-N); 1-Chloro-2,4-dinitrobenzene (VYZAHLCBVHPDDF-UHFFFAOYSA-N); 1-Dodecyl-4-nonylpyridinium chloride (LPAFDDIIVPMWRB-UHFFFAOYSA-M); 1-Formylisoquinoline thiosemicarbazone (FQWITMQQUMTMRE-AUWJEWJLSA-N); 1-Hexadecyl-3-methylimidazolium chloride (ZCPPLZJPPBIWRU-UHFFFAOYSA-M); 1-Methyl-3-octadecylimidazolium bis(trifluoromethylsulfonyl)imide (DMEDSJKEUMLSKF-UHFFFAOYSA-N); 1-Methyl-3-octadecylimidazolium hexafluorophosphate (DLCJUSZZKVAPPJ-UHFFFAOYSA-N); 1-Methyl-3-octadecylimidazolium tris(pentafluoroethyl)trifluorophosphate (MWCBXOFCMFFOQR-UHFFFAOYSA-N); 1-Methyl-3-phenylanthra[1,9-bc]thiopyran-3-ium chloride (VMKSVEZAPHWOQO-UHFFFAOYSA-M); 1-Nitro-9-(hydroxyethylamino)-acridine hydrochloride (WGUCIXFYKBZJBZ-UHFFFAOYSA-N); 1-Phenyl-2,3,4,5-tetrahydro-1H-benzo[d]azepine-7,8-diol hydrochloride (YEWHJCLOUYPAOH-UHFFFAOYSA-N); 1-Piperazinecarboselenoic acid, [1-(2-pyridinyl)ethylidene]hydrazide (XHOWLJGNFHYYAE-PQBPFAQHSA-N); 1-[(5-Methoxy-3-indolylidene)methylamino]-2-pentylguanidine (IKBKZGMPCYNSLU-UHFFFAOYSA-N); 1-[2-[(2-Methoxyacridin-9-yl)amino]ethylamino]propan-2-ol; hydrochloride (YXFRMZDXQIXPRL-UHFFFAOYSA-N); 1-[3-(10H-phenothiazine-3-carbonyl)indolizin-1-yl]ethanone (QAPYTONUTFHVJS-UHFFFAOYSA-N); 1-[4-(Diphenylmethyl)-1-piperazinyl]-3,3-diphenyl-1-propanone (VCPMZDWBEWTGNW-UHFFFAOYSA-N); 1-beta-D-Arabinofuranosyl-5-fluorocytosine (STRZQWQNZQMHQR-UHFFFAOYSA-N); 1-butyl-2-[(Z)-2-(2,4,6-trimethoxyphenyl)ethenyl]quinolin-1-ium; iodide (CCZVVSALPPSFTB-HPWRNOGASA-M); 1-cyclopentyl-3-[(E)-1-pyridin-2-ylethylideneamino]thiourea (VLBWJLNPSKLPBI-MHWRWJLKSA-N); 1-ethyl-2-((E)-3-((E)-1-ethylquinolin-2(1H)-ylidene)prop-1-en-1-yl)quinolin-1-ium iodide (QWYZFXLSWMXLDM-UHFFFAOYSA-M); 1-methyl-N-[(E)-1-pyridin-2-ylpropylideneamino]benzimidazol-2-amine (QUNQLOIANKWFSX-XDHOZWIPSA-N); 1-nitro-N-(3-piperidin-1-ylpropyl)acridin-9-amine; hydrochloride (RVMHSXWPLGSZOH-UHFFFAOYSA-N); 1-nitro-N-[2-(1-oxidopyridin-1-ium-2-yl)ethyl]acridin-9-amine; hydrochloride (LVWOOJMWUJOGTB-UHFFFAOYSA-N); 10,11-Methylenedioxy-20(RS)-camptothecin (RPFYDENHBPRCTN-UHFFFAOYSA-N); 10,11-Methylenedioxy-20S-camptothecin (RPFYDENHBPRCTN-NRFANRHFSA-N); 10-Aminocamptothecin (MVUUMBZAHAKPKQ-FQEVSTJZSA-N); 10-Chlorocamptothecin (MXIBGRFZFZXAKG-FQEVSTJZSA-N); 10-Hydroxy-7-trimethylsilylethylcamptothecin (FBOHVJOXVDWFTC-VWLOTQADSA-N); 10-Hydroxycamptothecin (HAWSQZCWOQZXHI-FQEVSTJZSA-N); 10-Methoxycamptothecin (KLFJSYOEEYWQMR-NRFANRHFSA-N); 1001645-58-4 (DTGRRMPPXCRRIM-UHFFFAOYSA-N); 1096708-71-2 (VWMJHAFYPMOMGF-ZCFIWIBFSA-N); 11-Hydroxy aclacinomycin A (DGBFPZUOWZFVHF-UHFFFAOYSA-N); 11-Hydroxy aclacinomycin X (LPFZXQBRNYTAKP-UHFFFAOYSA-N); 1144068-46-1 (QLHHRYZMBGPBJG-UHFFFAOYSA-N); 1173900-33-8 (IRTDIKMSKMREGO-OAHLLOKOSA-N); 1174046-72-0 (PDYXPCKITKHFOZ-UHFFFAOYSA-N); 118414-82-7 (QAOAOVKBIIKRNL-UHFFFAOYSA-N); 1194044-20-6 (MJQMRGWYPNIERM-HNNXBMFYSA-N); 1197958-12-5 (OVDSPTSBIQCAIN-UHFFFAOYSA-N); 1201902-80-8 (YTXSYWAKVMZICI-PVCZSOGJSA-N); 1214265-56-1 (APHGZZPEOCCYNO-UHFFFAOYSA-N); 1218942-37-0 (RGYQPQARIQKJKH-UHFFFAOYSA-N); 1234703-40-2 (QIIVJLHCZUTGSD-CUBQBAPOSA-N); 1243244-14-5 (XXYGTCZJJLTAGH-UHFFFAOYSA-N); 1254473-85-2 (GKJCVYLDJWTWQU-DUXPYHPUSA-N); 1257628-77-5 (TZKBVRDEOITLRB-UHFFFAOYSA-N); 1260907-17-2 (AAAQFGUYHFJNHI-SFHVURJKSA-N); 12677-11-1 (NKGFIFLSQXHQOK-MDZWDICZSA-N); 1268524-70-4 (DNVXATUJJDPFDM-KRWDZBQOSA-N); 1271738-62-5 (SRQYLNYQAPCPIR-UHFFFAOYSA-N); 129830-38-2 (IDDDVXIUIXWAGJ-DDSAHXNVSA-N); 13-Methoxydihydronitidine (SZEOMBMBHCBJJE-UHFFFAOYSA-N); 130332-27-3 (FVQSSYMRZKLFDR-ZABPBAJSSA-N); 134742-19-1 (JNGQUJZDVFZPEN-UHFFFAOYSA-N); 1365267-27-1 (ONPGOSVDVDPBCY-CQSZACIVSA-N); 138154-39-9 (CUNDRHORZHFPLY-UHFFFAOYSA-N); 1384426-12-3 (NIPUPOUEGOSAAO-OWOJBTEDSA-N); 138935-52-1 (ZFICSRNQVIMLDY-KRWDZBQOSA-N); 138935-58-7 (KKOZIXJFGMEJCY-NTKDMRAZSA-N); 138964-86-0 (WHTUBPJAGYKOQP-NFBKMPQASA-N); 138964-88-2 (KXSJHSREHJDQJT-UGSOOPFHSA-N); 139987-54-5 (BRVFNEZMTRVUGW-QFIPXVFZSA-N); 14-Deoxyandrographolide (GVRNTWSGBWPJGS-YSDSKTICSA-N); 14-methoxy-epothilone D (HQZJRAPNHNYEMZ-UPBMFQQMSA-N); 1414943-88-6 (ACWKGTGIJRCOOM-UHFFFAOYSA-N); 1421227-52-2 (KIKOYRNAERIVSJ-UHFFFAOYSA-N); 147098-20-2 (Calcium) (LALFOYNTGMUKGG-JGMJEEPBSA-L); 15-Deacetylsergeolide (NPWJHTWCKHUSAM-UHFFFAOYSA-N); 153587-01-0 (YLFMCMWKHSDUCT-UHFFFAOYSA-N); 1554458-53-5 (WNEILUNVMHVMPH-UHFFFAOYSA-N); 1570496-34-2 (HWJWNWZJUYCGKV-UHFFFAOYSA-N); 1578245-44-9 (SGWLRDAOCLITOM-UHFFFAOYSA-N); 15986-26-2 (OZKRFLRSUHXGBX-UHFFFAOYSA-N); 1610358-53-6 (YZCUMZWULWOUMD-UHFFFAOYSA-N); 1616632-77-9 (VLULRUCCHYVXOH-UHFFFAOYSA-N); 164178-33-0 (JHOTYHDSLIUKCJ-UHFFFAOYSA-N); 164658-13-3 (IYNDTACKOAXKBJ-UHFFFAOYSA-N); 17-Aminodemethoxygeldanamycin (XYFFWTYOFPSZRM-FFFKWXGKSA-N); 17-Desmethylgeldanamycin (AFFSZNHAULCEKY-AFGHJBDBSA-N); 18-Benzoyloxy-19-benzoylfuranotriptolide (KVKIMCYNPUCLPB-FZIJBHDVSA-N); 183499-57-2 (JNUGFGAVPBYSHF-UHFFFAOYSA-N); 192705-79-6 (NHJSWORVNIOXIT-UHFFFAOYSA-N); 193551-21-2 (ZQUSFAUAYSEREK-UHFFFAOYSA-N); 1H-Azepine-1-thiocarboxylic acid, hexahydro-, (2-pyridylmethylene)hydrazide (YELXJUBURDMCLZ-RVDMUPIBSA-N); 1H-Benzimidazole, 6-chloro-1-(2-chloroethyl)-2-(chloromethyl)-(AKNZHAUTXUWGGO-UHFFFAOYSA-N); 1H-Pyrazolo[4,3-d]pyrimidine, 7-amino-3-beta-D-ribofuranosyl-(KBHMEHLJSZMEMI-UHFFFAOYSA-N); 2′-Bromo-4′-epi-daunorubicin (RNTSDANIDLGPLQ-ZFXDRNAVSA-N); 2′-Deoxy-5-(trifluoromethyl)uridine (VSQQQLOSPVPRAZ-UHFFFAOYSA-N); 2′-Glutaryltaxol-3-(dimethylamino)propylamide (ZMYCRVJILUQQGH-QHKUZATPSA-N); 2(1H)-Pyridinone, 1-butyl-3-chloro-4-(4-phenyl-1-piperidinyl)-(HYOGJHCDLQSAHX-UHFFFAOYSA-N); 2(1H)-Pyrimidinone, 1-beta-D-ribofuranosyl-(RPQZTTQVRYEKCR-JJFBUQMESA-N); 2, 2′-(4-Ethoxyphenyl)-5-(4-methyl-1-piperazinyl)-, trihydrochloride (OTMWRDPOAAYNCR-UHFFFAOYSA-N); 2,10-Anthrylenedimethylene)-bis[2-thiopseudourea]dihydrochloride (DEYDZSUKTLIUOZ-UHFFFAOYSA-N); 2,2′-Cyclocytidine hydrochloride (KZOWNALBTMILAP-UHFFFAOYSA-N); 2,2′:6′,2″-Terpyridine (DRGAZIDRYFYHIJ-UHFFFAOYSA-N); 2,2-Dibromo-3,4-dihydronaphthalen-1(2H)-one (XJUWICWPKZOCJP-UHFFFAOYSA-N); 2,3-Dichloro-2,3-dihydronaphthalene-1,4-dione (BWLBTSHIHKJPIQ-UHFFFAOYSA-N); 2,3-Dihydro-2-(3-methoxyphenyl)-6-pyrrolizinoquinolin-4(1H)-one (UZLQYIIQUWXCBD-UHFFFAOYSA-N); 2,3-Dihydroxypropyl octanoate (GHBFNMLVSPCDGN-UHFFFAOYSA-N); 2,3-Dimercaptosuccinic acid (ACTRVOBWPAIOHC-UHFFFAOYSA-N); 2,4(1H,3H)-Pyrimidinedione, 5-fluoro-1-(tetrahydro-2-furanyl)-, (R)-(WFWLQNSHRPWKFK-ZCFIWIBFSA-N); 2,4,6-Tribromophenol (BSWWXRFVMJHFBN-UHFFFAOYSA-N); 2,5-Bis(1-aziridinyl)hydroquinone (XPZMWTZEBALMMC-UHFFFAOYSA-N); 2,6-Dibromo-4-[(6,7-dimethoxyquinazolin-4-yl)amino]phenol (YVCXQRVVNQMZEI-UHFFFAOYSA-N); 2,6-Dichloro-9-[2-[2-(2,6-dichloropurin-9-yl)ethoxy]ethyl]purine (LYZSZSXPVJPCGO-UHFFFAOYSA-N); 2,7-bis(piperidin-1-ylsulfonyl)-9H-fluoren-9-one oxime (XYZXEEIUKQGUHB-UHFFFAOYSA-N); 2,8]Phenanthroline-1,3,6,8-tetraone bis tosylate salt (UGTQQMWDKQVUJJ-UHFFFAOYSA-N); 2-((1-(2,N-dimethyl-1-ethanamine-, hydrochloride salt (FSIBYCVOFLVKGN-UHFFFAOYSA-N); 2-(1-Hexyloxyethyl)-2-devinyl pyropheophorbide-a (PUUBADHCONCMPA-USOGPTGWSA-N); 2-(1-Naphthyl)-5-methyl-1,8-naphthyridin-4(1H)-one (DAYKXJRPLWUPRH-UHFFFAOYSA-N); 2-(1H-indol-6-yl)-4-(3,4,5-trimethoxyphenyl)-1H-imidazo[4,5-c]pyridine (SQSZRGOISCZCTQ-UHFFFAOYSA-N); 2-(2-Methyl-5-nitro-1H-imidazol-1-yl)ethylsulfamide (AVSMSXHPIYIKIJ-UHFFFAOYSA-N); 2-(2-chloro-4-iodophenylamino)-N-cyclopropylmethoxy-3,4-difluorobenzamide (GFMMXOIFOQCCGU-UHFFFAOYSA-N); 2-(2-fluorophenyl)-6-(pyrrolidin-1-yl)quinolin-4(1H)-one (KFKXUFWYUQWHNT-UHFFFAOYSA-N); 2-(3-Dimethylamino-phenyl)-6-methoxy-1H-quinolin-4-one (UOIMUFPOYNIRLB-UHFFFAOYSA-N); 2-(3-Fluorophenyl)-5,7-dimethyl-1,8-naphthyridin-4(1H)-one (JNJKKBAULISGAT-UHFFFAOYSA-N); 2-(3-Methoxy-phenyl)-6-morpholin-4-yl-1H-quinolin-4-one (BSCMVEMZGHZNBZ-UHFFFAOYSA-N); 2-(3-fluorophenyl)-6-methyl-1,8-naphthyridin-4(1H)-one (AMJPGRQDFBNSON-UHFFFAOYSA-N); 2-(3-fluorophenyl)-7-methyl-1,8-naphthyridin-4(1H)-one (AFJQIXFUZDUCHX-UHFFFAOYSA-N); 2-(4-(Hydroxyimino)cyclohexa-2,5-dien-1-ylidene)-2-phenylacetonitrile (CEQPEKHLBZNDQM-UHFFFAOYSA-N); 2-(4-Formyl(2-((methylsulfonyl)oxy)ethyl)anilino)ethyl methanesulfonate (DCAJFQDHDVTBOS-UHFFFAOYSA-N); 2-(4-Methylphenyl)-5-phenyl-7-piperazin-1-yl-[1,3]oxazolo[4,5-d]pyrimidine (FOFVPNMYBLVSPD-UHFFFAOYSA-N); 2-(4-Morpholinyl)-8-phenyl-4H-1-benzopyran-4-one (CZQHHVNHHHRRDU-UHFFFAOYSA-N); 2-(4-amino-1-isopropyl-1H-pyrazolo[3,4-d]pyrimidin-3-yl)-1H-indol-5-ol (MFAQYJIYDMLAIM-UHFFFAOYSA-N); 2-(5-chloro-2-fluorophenyl)-N-(pyridin-4-yl)pteridin-4-amine (BERLXWPRSBJFHO-UHFFFAOYSA-N); 2-(5-fluoro-2-methyl-1H-indol-3-yl)-1H-imidazo[4,5-f][1,10]phenanthroline (NIRXBXIPHUTNNI-UHFFFAOYSA-N); 2-(Carboxy proxyl)-decyltriphenylphosphonium bromide (NTGAQMJUWCLECA-UHFFFAOYSA-M); 2-(Naphthalen-1-yl)-2,3-dihydroquinazolin-4(1h)-one (XKEIBCWTOUEGSH-UHFFFAOYSA-N); 2-(Sec-butyldisulfanyl)-1h-imidazole (BPBPYQWMFCTCNG-UHFFFAOYSA-N); 2-Amino-4-phenyl-4H-benzo[h]chromene-3-carbonitrile (BWJBHUUBGJLMMX-UHFFFAOYSA-N); 2-Amino-6-mercaptopurine riboside (OTDJAMXESTUWLO-UHFFFAOYSA-N); 2-Aminoethyl diphenylborinate (BLZVCIGGICSWIG-UHFFFAOYSA-N); 2-Bromo-4-[(6,7-dimethoxyquinazolin-4-yl)amino]phenol (CBIAKDAYHRWZCU-UHFFFAOYSA-N); 2-Bromoleptoclinidinone (CPXBDTZAJSRIKJ-UHFFFAOYSA-N); 2-Butenamide,6-dichlorophenyl)-4-oxo-(ZYIKIIOWBLFSTC-SNAWJCMRSA-N); 2-Butoxy-N-(2-diethylaminoethyl)cinchoninamide hydrochloride (IVHBBMHQKZBJEU-UHFFFAOYSA-N); 2-Chloro-9,10-diphenyl-9,10-dihydroacridin-9-ol (OSBOCAQJGKHPPR-UHFFFAOYSA-N); 2-Dimethylaminoethyl-2-methyl-benzhydryl ether citrate (MMMNTDFSPSQXJP-UHFFFAOYSA-N); 2-Ethoxy-3,17beta-dihydroxy-1,3,5(10)-estratrien-6-one oxime (SMKGGPJGMFBQDP-ZMMLKIRESA-N); 2-Ethynyl-4(3H)-quinazolinone (NXANOEQIIMPYKO-UHFFFAOYSA-N); 2-Fluoro-4-[5-(4-methoxyphenyl)thiophen-2-yl]benzenecarboximidamide (XEQZCHURPCKGAW-UHFFFAOYSA-N); 2-Mercaptobenzothiazole (YXIWHUQXZSMYRE-UHFFFAOYSA-N); 2-Methoxyestradiol (CQOQDQWUFQDJMK-SSTWWWIQSA-N); 2-Methyl-3-(phenyltelluro)-1,4-naphthoquinone (BKVMNWGUHUWTBP-UHFFFAOYSA-N); 2-Methyl-4-nitroquinoline 1-oxide (YRPLTHBJMGSMKH-UHFFFAOYSA-N); 2-Methyl-6-(phenylethynyl)pyridine (NEWKHUASLBMWRE-UHFFFAOYSA-N); 2-Morpholin-4-ylethyl 5,12-dioxoindolizino[2,3-g]quinoline-6-carboxylate (WLAKLXCQDUPBRG-UHFFFAOYSA-N); 2-Morpholino-6-(thianthren-1-yl)-4H-pyran-4-one (XRKYMMUGXMWDAO-UHFFFAOYSA-N); 2-N,3-N-bis(3-ethynylphenyl)-6-nitroquinoxaline-2,3-diamine (WSBKMBVHKOZASO-UHFFFAOYSA-N); 2-Naphthalen-1-yl-1H-[1,8]naphthyridin-4-one (PPEYNRXCXSJVHV-UHFFFAOYSA-N); 2-Naphthalenecarboxamide, 3-hydroxy-N-(4-nitrophenyl)-(ANHISTOUUYKCMS-UHFFFAOYSA-N); 2-Nitro-7-methoxynaphtho[2,1-b]furan (BRRIJZQYTOFDAF-UHFFFAOYSA-N); 2-Piperidin-1-ylethyl 5,12-dioxoindolizino[2,3-g]quinoline-6-carboxylate (OATANYSNZPXVHP-UHFFFAOYSA-N); 2-Propenyl estradiol (ZRVQFPQIVGUHNL-POAGYTCXSA-N); 2-Pyridin-2-ylethyl 5,12-dioxoindolizino[2,3-g]quinoline-6-carboxylate (XHSLXZCUAQQCEJ-UHFFFAOYSA-N); 2-Pyridin-4-yl-1,5,6,7-tetrahydropyrrolo[3,2-c]pyridin-4-one (DKXHSOUZPMHNIZ-UHFFFAOYSA-N); 2-Pyridinecarboxaldehyde, (4-methyl-2-quinolinyl)hydrazone (IGPVPTZWRCVXDL-WOJGMQOQSA-N); 2-Thiouracil (ZEMGGZBWXRYJHK-UHFFFAOYSA-N); 2-[(7-Methoxy-1-nitro-acridin-9-yl)amino]ethanol (QKERPOLKEQREML-UHFFFAOYSA-N); 2-[(E)-{2-[(4-benzylpiperazin-1-ium-1-yl)acetyl]hydrazinylidene}methyl]-6-(prop-2-en-1-yl)phenolate (YQNRVGJCPCNMKT-LFVJCYFKSA-N); 2-[(Z)-heptadec-10-enyl]-6-methoxybenzene-1,4-diol (BDWQNYPENCTULP-HJWRWDBZSA-N); 2-[2-(4-Nonylphenoxy)ethoxy]ethanol (BLXVTZPGEOGTGG-UHFFFAOYSA-N); 2-[2-[(1-Nitroacridin-9-yl)amino]ethylamino]ethanol (WWCHZSZUXCSNOQ-UHFFFAOYSA-N); 2-[3-[(2,3-Dimethoxy-6-nitroacridin-9-yl)amino]propylamino]ethanol; chloride ion (BRGPTRWGORGSOA-UHFFFAOYSA-M) salts; 2-[3-[(E)-(2-nitrofluoren-9-ylidene)methyl]pyridin-2-yl]oxyethanamine (PSTOZJZIPPUYHT-XDHOZWIPSA-N); 2-[6-(4-Chlorophenoxy)hexyl]-1-cyano-3-pyridin-4-ylguanidine (BOIPLTNGIAPDBY-UHFFFAOYSA-N); 2-[[5-Methoxy-1-[4-(trifluoromethyl)phenyl]pentylidene]amino]oxyethanamine (CJOFXWAVKWHTFT-UHFFFAOYSA-N); 2-allyl-6-morpholin-4-yl-1H-benzo[de]isoquinoline-1,3(2H)-dione (HXBXMNCIDQBCHE-UHFFFAOYSA-N); 2-amino-4-(3-methoxyphenyl)-4H-benzo[h]chromene-3-carbonitrile (QMOAGEFZQCHUSG-UHFFFAOYSA-N); 2-amino-4-(thiophen-2-yl)-4H-benzo[h]chromene-3-carbonitrile (RLDHRFZYYHWZQU-UHFFFAOYSA-N); 2-amino-7-methoxy-4-(3,4,5-trimethoxyphenyl)-4H-chromene-3-carbonitrile (MZLUGYPKQSBGDQ-UHFFFAOYSA-N); 2-amino-7-methoxy-4-(3-methoxyphenyl)-4H-chromene-3-carbonitrile (WKLKYONGSSJYLW-UHFFFAOYSA-N); 2-amino-N-[[4-(5-bromopyrimidin-2-yl)oxy-3-chlorophenyl]carbamoyl]benzamide (LRPFTPYFADHGQJ-UHFFFAOYSA-N); 2-beta-D-Ribofuranosyl-4-thiazolecarboxamide (FVRDYQYEVDDKCR-UHFFFAOYSA-N); 2-chloro-N-(4-methoxyphenyl)-N,5-dimethylpyrrolo[3,2-d]pyrimidin-4-amine (DHGLPKCITJOLFP-UHFFFAOYSA-N); 2-chloro-N-[4-(3-prop-2-ynoxyanilino)quinazolin-6-yl]acetamide (GUSWAPQBWHGWLI-UHFFFAOYSA-N); 2-chloro-N-[6-[(2-chloroacetyl)amino]-9,10-dioxoanthracen-2-yl]acetamide (UOWJONPWYDMJFO-UHFFFAOYSA-N); 2-ethynyl-N-(5-methyl-1H-pyrazol-3-yl)quinazolin-4-amine (LBMRCYHUGLYLPR-UHFFFAOYSA-N); 2-methoxy-5-[3-(3,4,5-trimethoxyphenyl)-1H-pyrazol-4-yl]aniline (FZSKMZZJIONNJA-UHFFFAOYSA-N); 2-{2-[4-(Dimethylamino)phenyl]ethenyl}-1-methylnaphtho[1,2-d][1,3]thiazol-1-ium iodide (IKAPOHKDZVIPFQ-UHFFFAOYSA-M); 205678-31-5 (KSSPHFGIOASRDE-HNNXBMFYSA-N); 209481-20-9 (LOCQRDBFWSXQQI-UHFFFAOYSA-N); 2095719-92-7 (BVRGQPJKSKKGIH-PUAOIOHZSA-N); 21090-42-6 (YZCAFRFEHKZTQT-UHFFFAOYSA-N); 219580-11-7 (DXCUKNQANPLTEJ-UHFFFAOYSA-N); 219773-55-4 (GZGLPBNOIFLLRE-UHFFFAOYSA-N); 22256-94-6 (LYPULEQVVXPJJR-UHFFFAOYSA-N); 232271-19-1 (DRSJLVGDSNWQBI-SFHVURJKSA-N); 245520-69-8 (AVXQPEKZIGPIJW-UHFFFAOYSA-N); 252003-65-9 (HXHAJRMTJXHJJZ-UHFFFAOYSA-N); 252917-06-9 (AQGNHMOJWBZFQQ-UHFFFAOYSA-N); 260415-63-2 (VAARYSWULJUGST-UHFFFAOYSA-N); 28380-24-7 (DANUORFCFTYTSZ-UHFFFAOYSA-N); 284028-89-3 (KLGQSVMIPOVQAX-UHFFFAOYSA-N); 293754-55-9 (SGIWFELWJPNFDH-UHFFFAOYSA-N); 2MD (UHMPCVGLSKFXHR-NAQZCRMNSA-N); 3′,5′-Dichloromethotrexate (MCEHFIXEKNKSRW-UHFFFAOYSA-N); 3′-(p-Chlorobenzoyl)spiro(indoline-3,2′-oxiran)-2-one (JGPRLTQNSPLADH-UHFFFAOYSA-N); 3′-C-Ethynylcytidine (JFIWEPHGRUDAJN-DYUFWOLASA-N); 3′-Carboxypropionyl-(Z)-combretastatin A-4 (BRHUMXPSUSFJQJ-WAYWQWQTSA-N); 3′-O-Methyl-evomonoside (VPUNMTHWNSJUOG-VZCPFXLZSA-N); 3,14-Dihydroxy-19-oxobufa-4,20,22-trienolide (XMFOZWPYGNDNKK-UHFFFAOYSA-N); 3,3′-Diindolylmethane (VFTRKSBEFQDZKX-UHFFFAOYSA-N); 3,3-Bis(8-hydroxy-7-methyl-5-quinolyl)isobenzofuran-1-one (KBNVZXWRVJSFSI-UHFFFAOYSA-N); 3,3-Dibutyl-6-nitro-2,4,3-benzodioxastannepine-1,5-dione (HPRWXVGSBBPEPP-UHFFFAOYSA-L); 3,4,5,4′-Tetramethoxystilbene (GGFQQRXTLIJXNY-AATRIKPKSA-N); 3,4,5-Tris(phenylsulfanyl)pyridine-2,6-dicarbonitrile (MMEWWNCMPUGYOO-UHFFFAOYSA-N); 3,4-Diaminopyridine (OYTKINVCDFNREN-UHFFFAOYSA-N); 3,4-Dibutoxy-3-cyclobutene-1,2-dione (XBRWELTXMQSEIN-UHFFFAOYSA-N); 3,5-bis[(E)-2-(4-hydroxy-3-methoxy-phenyl)vinyl]pyrazole-1-carboxamide (HVTSVGLLJGNWME-FCXRPNKRSA-N); 3,6-Acridinediamine, sulfate (2:1) (YADYXCVYLIKQJX-UHFFFAOYSA-N); 3,6-Bis(methylsulfanyl)-1,2-dihydro-1,2,4,5-tetrazine (LNYJEPPVBXMFNT-UHFFFAOYSA-N); 3,6-Diamino-10-methylacridinium chloride (KKAJSJJFBSOMGS-UHFFFAOYSA-N); 3,8,9-trimethoxy-5H-isoindolo[2,1-a]quinoxalin-6-one (CNSTVRRUULEAPC-UHFFFAOYSA-N); 3-(1-Anilinoethylidene)-5-benzylpyrrolidine-2,4-dione (IHXKJZIDPGITHW-UHFFFAOYSA-N); 3-(1-Methylpyrrolidin-2-yl)pyridine (SNICXCGAKADSCV-UHFFFAOYSA-N); 3-(3,4,5-Trimethoxyphenyl)-4-(2-naphthyl)-2,5-dihydrofuran-2-one (LDGKCQXGMWOFDM-UHFFFAOYSA-N); 3-(3-Cyclopentyloxy-4-methoxyphenyl)-3-(1,3-dioxoisoindol-2-yl)propanamide (DDYUBCCTNHWSQM-UHFFFAOYSA-N); 3-(3-Pyridinyl)-1-(4-pyridinyl)-2-propen-1-one (UOWGYMNWMDNSTL-ONEGZZNKSA-N); 3-(3-aminophenyl)-4-(1-methyl-1H-indol-3-yl)-1H-pyrrole-2,5-dione (RGTAEYDIDMGJLX-UHFFFAOYSA-N); 3-(3-chlorophenyl)-1-(1-methyl-4-oxo-5H-imidazol-2-yl)urea (DWPQODZAOSWNHB-UHFFFAOYSA-N); 3-(4-(Dimethylamino)phenyl)-2-phenylacrylonitrile (HYAJIHDALOHMPT-FOWTUZBSSA-N); 3-(4-Amino-2-chlorophenyl)-6-chloro-1,3-benzoxazine-2,4-dione (IYYYZUWUOINRSE-UHFFFAOYSA-N); 3-(4-Methoxyphenyl)-2-(3,4,5-trimethoxyphenyl)prop-2-enenitrile (IHKBGXGPWWAEPU-OQLLNIDSSA-N); 3-(4-Methoxyphenyl)-8H-thieno[2,3-b]pyrrolizin-8-one (STBQKICVQXGVLM-UHFFFAOYSA-N); 3-(5-Fluoro-1H-indol-3-yl)pyrrolidine-2,5-dione (MXKLDYKORJEOPR-UHFFFAOYSA-N); 3-(Chloromethyl)-6-hydroxy-1-((5,7-trimethoxyindol-2-yl)carbonyl)indoline (KFMLAUJMEDHHPN-UHFFFAOYSA-N); 3-(o-(Bis(beta-chloroethyl)amino)phenyl)-D,L-alanine (ZJHNCWIVJDCKSB-UHFFFAOYSA-N); 3-Cyclohexyl-1-(2-fluoroethyl)-1-nitrosourea (SIJUHDYJDHLNLY-UHFFFAOYSA-N); 3-Demethyl-3-butyrylthiocolchicine (NXMUVIPICRBKOW-UHFFFAOYSA-N); 3-Demethyl-3-ethoxycarbonylthiocolchicine (LDZKAGVVCFBEMQ-UHFFFAOYSA-N); 3-Desmethylcolchicine (JRRUSQGIRBEMRN-HNNXBMFYSA-N); 3-Ethoxycarbonyl-N-trifluoroacetylthiocolchicine (GARIEDXBHKTNKL-UHFFFAOYSA-N); 3-Hydroxy-alpha-methyl-DL-tyrosine (CJCSPKMFHVPWAR-UHFFFAOYSA-N); 3-Methoxy-11-nitroso-5,6,6a,7-tetraaza-6aH-benzo[a]fluorene (CQNHUCFXHWOHSA-UHFFFAOYSA-N); 3-Methoxy-5H-8-methoxy-isoindolo[2,1-A]quinoxalin-6-one (KUWBJYLEYHPKQQ-UHFFFAOYSA-N); 3-Methoxy-5H-9-methoxy-isoindolo[2,1-A]quinoxalin-6-one (HGAHKZJDPQSSQB-UHFFFAOYSA-N); 3-Methyl-1-octadecylimidazolium chloride (LCXGSWXECDJESI-UHFFFAOYSA-M); 3-Methyl-2-methylsulfanylnaphtho[2,3-g]pteridine-4,6,11-trione (LFWGMTQWABHFPN-UHFFFAOYSA-N); 3-Methylthienyl-carbonyl-JNJ-7706621 (KQWTXAWKBUUOMU-UHFFFAOYSA-N); 3-Morpholin-4-ylpropyl 5,12-dioxoindolizino[2,3-g]quinoline-6-carboxylate (CMEJZBGTPMODEK-UHFFFAOYSA-N); 3-Piperidin-1-ylpropyl 5,12-dioxoindolizino[2,3-g]quinoline-6-carboxylate (DVZCMBJSRRXMRW-UHFFFAOYSA-N); 3-Pyrrolin-2-one, 4-hydroxy-5-(p-hydroxybenzyl)-3-(N-phenylacetimidoyl)-(CDSZPMMTWNONAM-UHFFFAOYSA-N); 3-[(1s,2s)-2-Chlorocyclohexyl]-1-(2-chloroethyl)-1-nitrosourea (AVQDABCAQFMRFT-YUMQZZPRSA-N); 3-[(3,5-dibromo-4-hydroxyphenyl)methylidene]-5-iodo-1H-indol-2-one (LMXYVLFTZRPNRV-UHFFFAOYSA-N); 3-[(3-(2-Carboxyethyl)-4-methylpyrrol-2-YL)methylene]-2-indolinone (JNDVEAXZWJIOKB-JYRVWZFOSA-N); 3-[(E)-3-(2-ethoxyphenyl)-3-oxoprop-1-enyl]-1H-quinolin-2-one (SEISRPMGAKDAMQ-VAWYXSNFSA-N); 3-[3,5-Bis(trifluoromethyl)phenyl]-6-chloro-1,3-benzoxazine-2,4-dione (NZMFXIDRQQEOIC-UHFFFAOYSA-N); 3-[[(3S)-1-cyanopyrrolidine-3-carbonyl]amino]isoquinoline-6-carboxamide (OPMHXWMODHWNRI-LBPRGKRZSA-N); 3-acetyl-5,6,8-trichloro-2-(3-fluoroanilino)-1H-quinolin-4-one (SUWZPIWLTGKRDK-UHFFFAOYSA-N); 3-acetyl-6,8-difluoro-2-(2,3,4-trifluoroanilino)-1H-quinolin-4-one (GFNFICLJUUISAM-UHFFFAOYSA-N); 3-hydroxy-8,9-dimethoxylisoindolo[2,1a]quinoxalin-6-(5H)-one (ZOWTUSVWBAYUSE-UHFFFAOYSA-N); 3-hydroxy-8,9-dimethylisoindolo[2,1a]quinoxalin-6-(5H)-one (OOFZMVGNPQLOOY-UHFFFAOYSA-N); 3-methoxy-8,9-dimethyl-5H-isoindolo[2,1-a]quinoxalin-6-one (PNRUIYJMOWEEFK-UHFFFAOYSA-N); 319460-94-1 (JXSVVZKPEDIRTN-DHZHZOJOSA-N); 32319-89-4 (LKCXGNNLVAZPIG-UHFFFAOYSA-N); 331771-20-1 (OGNYUTNQZVRGMN-UHFFFAOYSA-N); 336184-91-9 (DMRUQISFVFDWGE-UHFFFAOYSA-M); 348622-88-8 (ARJCBSRIPGJMAD-LLVKDONJSA-N); 38838-27-6 (NTJNSPIBKNYJBX-HNNXBMFYSA-N); 391210-10-9 (SUDAHWBOROXANE-SECBINFHSA-N); 3H-Indolium, 2-[3-(1,3-dihydro-1,3,3-trimethyl-2H-indol-2-ylidene)-1-propenyl]-1,3,3-trimethyl-, chloride (QCGOYKXFFGQDFY-UHFFFAOYSA-M); 3H-Phenoxazin-3-one, 7-hydroxy-, 10-oxide, sodium salt (IVGPGQSSDLDOLH-UHFFFAOYSA-M); 4′, Dihydrochloride (KMYZYBSGVCGXOT-UHFFFAOYSA-N); 4′-Thio-2′-deoxycytidine (MOMUJZRKXYLWMH-SHYZEUOFSA-N); 4(1H)-Quinolinone, 6-(1-pyrrolidinyl)-2-[3-(trifluoromethoxy)phenyl]-(IXOPCLABJYZIEL-UHFFFAOYSA-N); 4(1H)-Quinolinone,3-dihydro-6-(1-pyrrolidinyl)-(ZOEQEDMPQAHJBM-UHFFFAOYSA-N); 4,4′-[(4-Chlorophenyl)methanediyl]bis(n,n-dimethylaniline) (JGYFBXILBKWGBN-UHFFFAOYSA-N); 4,6-dimethyl-N-[(E)-1-pyridin-2-ylethylideneamino]quinolin-2-amine (MQAAKPITBPYSEY-KGENOOAVSA-N); 4,7-Dichloro-3-(2-(4-cyclopropylphenyl)-2-oxoethyl)-3-hydroxyindolin-2-one (ZWHNLSHDLKIXOG-UHFFFAOYSA-N); 4,8-Dichloro-6,11-dimethyl-5,6-dihydropyrimido[4,5-b][1]benzazepine (BJNRIFXVPSUSLV-UHFFFAOYSA-N); 4-(2,6-dichlorobenzamido)-N-(piperidin-4-yl)-1H-pyrazole-3-carboxamide (OVPNQJVDAFNBDN-UHFFFAOYSA-N); 4-(2-Methylquinazoline-4-yl)-7-methoxy-3,4-dihydroquinoxaline-2(1H)-one (LTTPRSPHFQPOMT-UHFFFAOYSA-N); 4-(2-Pyridinyl)-1-piperazinecarbothioic acid, dihydrochloride (IKTIHHRILHMURF-RANVTSCRSA-N); 4-(4-(benzo[d][1,3]dioxol-5-yl)-5-(pyridin-2-yl)-1H-imidazol-2-yl)benzamide (FHYUGAJXYORMHI-UHFFFAOYSA-N); 4-(4-Fluorophenyl)-2-(4-methylsulfinylphenyl)-5-(4-pyridyl)-1H-imidazole (CDMGBJANTYXAIV-UHFFFAOYSA-N); 4-(4-Methoxy-phenyl)-5-(3,4,5-trimethoxy-phenyl)-isoxazole (XUGSPERSQBCGJK-UHFFFAOYSA-N); 4-(4-chlorobenzyl)-1-(7H-pyrrolo[2,3-d]pyrimidin-4-yl)piperidin-4-amine (RZIDZIGAXXNODG-UHFFFAOYSA-N); 4-(4-chlorophenyl)-4-[4-(1H-pyrazol-4-yl)phenyl]piperidine (LZMOSYUFVYJEPY-UHFFFAOYSA-N); 4-(4-methoxyphenyl)-5-(3,4,5-trimethoxyphenyl)-1H-1,2,3-triazole (JHZMSGCUGQSNOA-UHFFFAOYSA-N); 4-(4-methoxyphenyl)-5-(3,4,5-trimethoxyphenyl)-1H-pyrazole (VYYMOJHULCKDCT-UHFFFAOYSA-N); 4-(7-methoxy-1,3-benzodioxol-5-yl)-5-(4-methoxyphenyl)-1H-1,2,3-triazole (DOZOVRQLBANESF-UHFFFAOYSA-N); 4-(Dimethylamino)-n-[7-(hydroxyamino)-7-oxoheptyl]benzamide (MXWDSZWTBOCWBK-UHFFFAOYSA-N); 4-({[(4-Tert-Butylphenyl)sulfonyl]amino}methyl)-N-(Pyridin-3-Yl)benzamide (NGQPRVWTFNBUHA-UHFFFAOYSA-N); 4-Amino-4-deoxy-10-ethyl-10-deazapteroyl-gamma-methylene glutamic acid (WABHENAZLPSQIE-UHFFFAOYSA-N); 4-Aminopteroylglutamic acid (TVZGACDUOSZQKY-UHFFFAOYSA-N); 4-Aminosalicylic acid (WUBBRNOQWQTFEX-UHFFFAOYSA-N); 4-Benzyl-2-methyl-1,2,4-thiadiazolidine-3,5-dione (JDSJDASOXWCHPN-UHFFFAOYSA-N); 4-Chloroquinolin-8-ol (DEPRJVQVGXOXRN-UHFFFAOYSA-N); 4-Demethoxyadriamycin HCl (XVXGYZFARCOVHS-UHFFFAOYSA-N); 4-Hydroperoxycyclophosphamide (VPAWVRUHMJVRHU-UHFFFAOYSA-N); 4-Hydroxyaminoquinoline 1-oxide monohydrochloride ion (QSXDAZCGWHRQME-DQMXGCRQSA-M) salts; 4-Methyl-5-(2-(4-morpholinophenylamino)pyrimidin-4-yl)thiazol-2-amine (GPSZYOIFQZPWEJ-UHFFFAOYSA-N); 4-Methyl-N-(3,4,5-trimethoxybenzyl)aniline hydrochloride (YNUQVPKYSQEUIO-UHFFFAOYSA-N); 4-Methyl-N-[(E)-1-pyridin-2-ylethylideneamino]quinolin-2-amine (CVDUGZRMCGEUJY-DEDYPNTBSA-N); 4-Nitroquinoline N-oxide (YHQDZJICGQWFHK-UHFFFAOYSA-N); 4-[(3-Bromophenyl)amino]-6-acrylamidoquinazoline (HTUBKQUPEREOGA-UHFFFAOYSA-N); 4-[2-(Ethylamino)quinazolin-4-yl]-7-methoxy-1,3-dihydroquinoxalin-2-one (XLBSCEPOSNTQJB-UHFFFAOYSA-N); 4-[3-(4-Methoxyphenyl)-5-phenyl-3,4-dihydropyrazol-2-yl]benzenesulfonamide (QBNZBMVRFYREHK-UHFFFAOYSA-N); 4-[3-Hydroxyanilino]-6,7-dimethoxyquinazoline (BNDYIYYKEIXHNK-UHFFFAOYSA-N); 4-[4-(4-Hydroxyphenyl)hex-3-en-3-yl]phenol (RGLYKWWBQGJZGM-UHFFFAOYSA-N); 4-[4-(dibutylamino)benzenecarboximidoyl]-N,N-dimethylaniline; hydrochloride (CJRPCHQWAFGBMM-UHFFFAOYSA-N); 4-[4-[[(1R)-1-phenylethyl]amino]-7H-pyrrolo[4,5-e]pyrimidin-6-yl]phenol (XRYJULCDUUATMC-CYBMUJFWSA-N); 4-[Bromo-(4-hydroxy-3-nitrophenyl)arsanyl]-2-nitrophenol (OMJNTTGUVBGBLD-UHFFFAOYSA-N); 4-[bis(1H-pyrrol-2-yl)methyl]-6-methoxychromen-2-one (SOEWKZHDGLLZRP-UHFFFAOYSA-N); 4-cycloheptyl-N-(1-pyridin-2-ylethylideneamino)piperazine-1-carbothioamide (ZGAVNXQAROBPJG-LTGZKZEYSA-N); 4-dodecyl-N-(1,3,4-thiadiazol-2-yl)benzenesulfonamide (BYWWNRBKPCPJMG-UHFFFAOYSA-N); 40066-33-9 (VADOLDYAUSTMFK-UHFFFAOYSA-N); 405911-17-3 (NMPUWJFHNOUNQU-UHFFFAOYSA-N); 442556-59-4 (PEPLRSOXCXBORF-UHFFFAOYSA-N); 452105-23-6 (CTNPALGJUAXMMC-PMFHANACSA-N); 476159-98-5 (WPOXAFXHRJYEIC-UHFFFAOYSA-N); 482306-32-1 (KGFYHTZWPPHNLQ-UHFFFAOYSA-N); 497839-62-0 (OONFNUWBHFSNBT-HXUWFJFHSA-N); 5′-Chloro-3-hydroxy-2′-methyl-2-naphthanilide (XZOACPDZZYNJER-UHFFFAOYSA-N); 5′-Sulfamoyl-2-chloroadenosine (JHUGCRSKMUFKHR-UUOKFMHZSA-N); 5,10,10-Trimethylbenzo[b][1,4]benzazastannine (HDLOTGARUIVRDY-UHFFFAOYSA-N); 5,3′-Dihydroxy-3,6,7,8,4′-pentamethoxyflavone (GVLMRDDSMVCWKB-UHFFFAOYSA-N); 5,6,11-Trimethylpyrido[4,3-b]carbazole-9-carbonitrile (XURZIXYYIWUSBU-UHFFFAOYSA-N); 5,6-Dihydro-1,2,3,9-tetramethoxy-7H-dibenzo[a,c]cycloheptene-7-one (LCUPINWVEJYXRG-UHFFFAOYSA-N); 5,7-Dihydroxy-4-methylcoumarin (QNVWGEJMXOQQPM-UHFFFAOYSA-N); 5-(3-hydroxy-4-methoxyphenyl)-6-(3,5-trimethoxyphenyl)-2H-pyran-2-one (UKDQCIHVKSGIHE-UHFFFAOYSA-N); 5-(4-Methoxyphenyl)-4-(3,4,5-trimethoxyphenyl)-1,2-oxazole (SDEVLRKDBBUKIA-UHFFFAOYSA-N); 5-(4-chlorophenyl)-N-(3,5-dimethoxyphenyl)furan-2-carboxamide (VHKBTPQDHDSBSP-UHFFFAOYSA-N); 5-(4-methoxyphenyl)-4-(3,4,5-trimethoxyphenyl)-1H-pyrazole (XDEMMZMSHRWEAU-UHFFFAOYSA-N); 5-(9-Isopropyl-8-methyl-2-morpholino-9H-purin-6-yl)pyrimidin-2-amine (QYBGBLQCOOISAR-UHFFFAOYSA-N); 5-(Hydroxymethyl)-11-methyl-6H-pyrido(4,3-b)carbazole N-methylcarbamate (JEURWCFQOJUTQI-UHFFFAOYSA-N); 5-(p-Methoxyphenyl)-4-(3,4,5-trimethoxyphenyl)-1H-pyrazole-3-amine (KJOZDTMYYINTJW-UHFFFAOYSA-N); 5-Chloro-8-hydroxyquinoline (CTQMJYWDVABFRZ-UHFFFAOYSA-N); 5-Fluoro-2′-deoxyuridine (ODKNJVUHOIMIIZ-UHFFFAOYSA-N); 5-Fluorouracil arabinoside (FHIDNBAQOFJWCA-UHFFFAOYSA-N); 5-Hydroxypicolinaldehyde thiosemicarbazone (LJHGXGDHNOZLFT-XCVCLJGOSA-N); 5-Methoxy-9H-quinolino[4,3,2-de][1,10]phenanthrolin-9-one (LSUJEFUOJJOYDR-UHFFFAOYSA-N); 5-[(8-Hydroxyquinolin-5-yl)methyl]quinolin-8-ol (FKGLQHRCVKTDFC-UHFFFAOYSA-N); 5-[2-(4-Methoxyphenyl)ethyl]-1,2,3-trimethoxybenzene (AQBXGPHZDHQKJU-UHFFFAOYSA-N); 5-[5-(3,5-Dimethoxyphenyl)thiophen-2-yl]thiophene-2-carboximidamide (QBWGTMWTMGHCAC-UHFFFAOYSA-N); 5-[5-(4-Methoxyphenyl)thiophen-2-yl]thiophene-2-carboximidamide (JTDLPMKUDNXSPW-UHFFFAOYSA-N); 5-chloro-2-hydroxy-N-[4-(trifluoromethyl)-1,3-benzothiazol-2-yl]benzamide (LMBRUTDBWLTRCS-UHFFFAOYSA-N); 5-chloro-2-hydroxy-N-[5-(trifluoromethyl)pyrazin-2-yl]benzamide (GOSNEJFMQOZLHX-UHFFFAOYSA-N); 5-chloro-N-(2-fluoro-4-iodophenyl)-2-hydroxybenzamide (WVHDAHMWJTUXKU-UHFFFAOYSA-N); 5-chloro-N-(4-chloro-2-fluorophenyl)-2-hydroxybenzamide (BYOLJAWIFMRPOU-UHFFFAOYSA-N); 5-chloro-N-(4-methoxyphenyl)-N,1-dimethylpyrazolo[4,3-d]pyrimidin-7-amine (OXJPJFSQXVFWIL-UHFFFAOYSA-N); 5-chloro-N-[(E)-1-pyridin-2-ylethylideneamino]pyridin-2-amine (FIGHXLYRMLXCQO-CXUHLZMHSA-N); 5-hydroxy-L-tryptophan (LDCYZAJDBXYCGN-VIFPVBQESA-N); 5-methyl-2-phenyl-1H-1,8-naphthyridin-4-one (WGNSONPPEWQOMA-UHFFFAOYSA-N); 5-methyl-N-(4-piperidyl)-10H-indolo[3,2-b]quinolin-5-ium-11-amine chloride (GRLHUXVTIXQJHW-UHFFFAOYSA-N); 51387-68-9 (DINNMQMTOABTAT-UHFFFAOYSA-N); 51963-58-7 (HVOJSINFDBMIRX-UHFFFAOYSA-N); 5418-59-7 (FTCNBUOQORTZBK-UHFFFAOYSA-N); 55078-51-8 (QCDIRNSCHKOUIQ-CHHCPSLASA-N); 57710-57-3 (JESMSCGUTIEROV-ASHKBJFXSA-N); 57879-46-6 (MXQQSGPXGZJFFC-UHFFFAOYSA-M); 59987-97-2 (KHPAANXZKKTBNE-UHFFFAOYSA-N); 6,7-Bis(3-hydroxyphenyl)pteridine-2,4-diamine (UJIAQDJKSXQLIT-UHFFFAOYSA-N); 6,7-Dimethoxy-2-phenylquinoxaline (QNOXYUNHIGOWNY-UHFFFAOYSA-N); 6,8-Bis(benzylthio)octanoic acid (ZYRLHJIMTROTBO-UHFFFAOYSA-N); 6-(2-Hydroxy-3-methoxybenzyl)aminopurine-9-riboside (SJHYLQCPNOPCPE-UHFFFAOYSA-N); 6-(3-Aminopropyl)-5,11-dioxo-11H-indeno[1,2-c]isoquinoline Hydrochloride (NQWBFTIDOATMLE-UHFFFAOYSA-N); 6-(3-Fluorophenyl)-6,7-dihydro-[1,3]dioxolo[4,5-G]quinolin-8(5H)-one (IJYJIPDFDHBJTD-UHFFFAOYSA-N); 6-(3-Iodopropyl)-9-methoxy-3-nitroindeno[1,2-c]isoquinoline-5,11-dione (UUJPBZJHYIEZMS-UHFFFAOYSA-N); 6-(3-fluorophenyl)-5H-[1,3]dioxolo[4,5-g]quinolin-8-one (DWLFPUBOAWVQJC-UHFFFAOYSA-N); 6-(4-Tert-butylphenoxy)pyridin-3-amine (WHIWGRCYMQLLAO-UHFFFAOYSA-N); 6-(Cyclohexylmethoxy)-5-nitrosopyrimidine-2,4-diamine (DGWXOLHKVGDQLN-UHFFFAOYSA-N); 6-(Dimethylamino)-2-(3-methoxyphenyl)quinolin-4(1h)-one (QWTLGBXHTGTPMU-UHFFFAOYSA-N); 6-(Dimethylarsanylsulfanylmethyl)oxane-2,3,4,5-tetrol (MGSFMZYHFQRBQS-UHFFFAOYSA-N); 6-(cyclohexylmethoxy)-9H-purin-2-amine (MWGXGTJJAOZBNW-UHFFFAOYSA-N); 6-Amino-7-bromo-5,8-quinolinedione (XZBBOHUCBTXJQY-UHFFFAOYSA-N); 6-Aminonicotinamide (ZLWYEPMDOUQDBW-UHFFFAOYSA-N); 6-Aminotoyocamycin (GPEPWIICLLUDAS-UHFFFAOYSA-N); 6-Bromo-3-(2-fluoro-4-iodophenyl)-1,3-benzoxazine-2,4-dione (AOQSIKVVMDXCDF-UHFFFAOYSA-N); 6-Bromoindirubin-3′-oxime (SAQUSDSPQYQNBG-UHFFFAOYSA-N); 6-Chloro-2-methoxy-9-[(5-piperidinopentyl)amino]acridine (MRDUIMWRCOBGBZ-UHFFFAOYSA-N); 6-Chloro-3-(2-fluoro-4-iodophenyl)-1,3-benzoxazine-2,4-dione (XETMHLHLINEAAY-UHFFFAOYSA-N); 6-Chloro-3-(4-chloro-2-fluorophenyl)-1,3-benzoxazine-2,4-dione (UDMNFCGRSABOJK-UHFFFAOYSA-N); 6-Chloro-4-(3-fluoro-4-methoxyphenyl)coumarin (MPIKMKWERBRKSE-UHFFFAOYSA-N); 6-Chloro-4-[4-(methylamino)phenyl]coumarin (URDSXUQHONNYSK-UHFFFAOYSA-N); 6-Chloro-7-(2-morpholin-4-yl-ethylamino)quinoline-5,8-dione (BMKPVDQDJQWBPD-UHFFFAOYSA-N); 6-Diazo-5-oxo-norleucine (YCWQAMGASJSUIP-UHFFFAOYSA-N); 6-Ethyl-5,11-dimethyl-2-tetradecylpyrido[4,3-b]carbazol-2-ium-9-ol ion (HRRGDZJIKQYAFA-UHFFFAOYSA-O) salts; 6-Fluoro-3-(2-fluoro-4-iodophenyl)-1,3-benzoxazine-2,4-dione (DXXCIDIQQJAYHM-UHFFFAOYSA-N); 6-Mercaptopurine monohydrate (WFFQYWAAEWLHJC-UHFFFAOYSA-N); 6-Methoxy-2-(3-methoxy-phenyl)-1H-quinolin-4-one (FVTUSWUAAUJBRP-UHFFFAOYSA-N); 6-Methyl-2-(naphthalen-1-yl)-1,8-naphthyridin-4(1h)-one (KBQRHVUXLRZRAC-UHFFFAOYSA-N); 6-Methyl-2-(phenylazo)-3-pyridinol (LOCPVWIREQIGNQ-UHFFFAOYSA-N); 6-Methylthioinosine (ZDRFDHHANOYUTE-UHFFFAOYSA-N); 6-Pyrrolidin-1-yl-2-[3-(trifluoromethoxy)phenyl]quinolin-4-amine (JOGCPGCVMKCCRI-UHFFFAOYSA-N); 6-Thioinosine (NKGPJODWTZCHGF-UHFFFAOYSA-N); 6-[(1,4-dioxonaphthalen-2-yl)amino]-N-(3-phenyltellanylpropyl)hexanamide (SWOLWANYRGUGNY-UHFFFAOYSA-N); 6-amino-8-(3-methoxyphenyl)-8H-[1,3]dioxolo[4,5-g]chromene-7-carbonitrile (HLTIWUUAMXMQRC-UHFFFAOYSA-N); 6-chloro-2,3,4,9-tetrahydro-1H-carbazole-1-carboxamide (FUZYTVDVLBBXDL-UHFFFAOYSA-N); 6-chloro-2-naphthalen-1-yl-2,3-dihydro-1H-quinazolin-4-one (ZGIFEPWYKKDPTK-UHFFFAOYSA-N); 6-chloro-9-((4-methoxy-3,5-dimethylpyridin-2-yl)methyl)-9H-purin-2-amine (QULDDKSCVCJTPV-UHFFFAOYSA-N); 6-hydroxy-4-oxo-N-phenyl-2-sulfanylidene-1H-pyrimidine-5-carboxamide (GFYRZTLCYQQVHZ-UHFFFAOYSA-N); 6-methoxy-4-methyl-N-[(E)-1-pyridin-2-ylethylideneamino]quinolin-2-amine (JMBDCADTMYGNQO-FYJGNVAPSA-N); 6-methyl-4-[(E)-1H-pyrrol-2-yl(pyrrol-2-ylidene)methyl]chromen-2-one (HVEWUBXSHABGAI-KNTRCKAVSA-N); 602306-29-6 (WJRRGYBTGDJBFX-UHFFFAOYSA-N); 604769-01-9 (MUTBJZVSRNUIHA-UHFFFAOYSA-N); 6286-97-1 (UFESHPVTIVDMGM-UHFFFAOYSA-N); 6320-13-4 (IPSCFKKYZIVWAL-XGRJIHFXSA-M); 6320-32-7 (ISXULQMIXWTWJH-UHFFFAOYSA-M); 635318-11-5 (AVDUGNCTZRCAHH-MDASVERJSA-N); 63620-51-9 (SMEGTTUCKYOLLP-INIZCTEOSA-N); 64-86-8 (IAKHMKGGTNLKSZ-UHFFFAOYSA-N); 66568-82-9 (ACIAPWKOTHFOAA-XQMHFWANSA-N); 666260-75-9 (TWQYWUXBZHPIIV-UHFFFAOYSA-N); 66996-56-3 (QEORIOWOGNHTED-YMYQXXOFSA-N); 67106-76-7 (GDHDMMYJQUZEPN-MTDHEZBUSA-N); 67198-13-4 (VQLPLYSROCPWFF-QZTJIDSGSA-N); 691396-67-5 (CQHPWGQQERADRN-UHFFFAOYSA-N); 6H-Pyrido[4, 5,11-dimethyl-, dihydrochloride (MTHGSLOYRDMTHQ-UHFFFAOYSA-N); 6H-Pyrido[4, 9-methoxy-5,11-dimethyl-,dihydrochloride (VFENHZVXEDVOAK-UHFFFAOYSA-N); 6alpha-Senecioyloxychaparrinone (JLEOCIUEZOZFST-UHFFFAOYSA-N); 7-Bromodemethoxy geldanoxazone (GRRRVBFXHGBGOJ-DOMYYLLWSA-N); 7-(3,4-dimethoxybenzyl)-5,7-dihydro-4H-isoxazolo[5,4-e]isoindole (AQQPAZYXDDZERI-UHFFFAOYSA-N); 7-(6-bromo-1H-benzimidazol-2-yl)-N-hydroxy heptanamide (KGEJYSFIEAQAKU-UHFFFAOYSA-N); 7-(Benzylamino)-1,3,4,8-tetrahydropyrrolo[4,3,2-de]quinoline-8-one (PIQBKCOHWKHJBX-UHFFFAOYSA-N); 7-(beta-D-ribofuranosyl)-4-aminothieno[3,4-d]pyrimidine (VTCXCOOTIXNZFE-UHFFFAOYSA-N); 7-Amino-6-chloro-5,8-dioxonaphthalene-2-sulfonamide (ICJBRQPJQVOTDH-UHFFFAOYSA-N); 7-Con-O-methylnogarol (LWYJUZBXGAFFLP-KIWNRLSNSA-N); 7-Ethyl-10,11-methylenedioxy-20-S-camptothecin (VPGWXOVUCCDEPV-QHCPKHFHSA-N); 7-Ethyl-10-hydroxycamptothecin (FJHBVJOVLFPMQE-QFIPXVFZSA-N); 7-Ethyl-9-amino-10,11-methylenedioxy-20(S)-camptothecin (SVBVRHFMDMNEON-QHCPKHFHSA-N); 7-Hydroxystaurosporine (PBCZSGKMGDDXIJ-HQCWYSJUSA-N); 7-Methoxy-2-(trichloromethyl)chromen-4-one (LPLSLEOFBMPIFG-UHFFFAOYSA-N); 7-Methoxy-8-(4-2-methoxy-4-[(10-oxo-9,10-dihydro-9-nthracenyliden)methyl](RPDVVSQGCUDZGT-UHFFFAOYSA-N); 7-O-Methylepinogalarol (HXCSJGHKDZKLKV-RNFADZNJSA-N); 7-[(3-Phenoxyphenyl)(pyridin-2-ylamino)methyl]quinolin-8-ol (WJNJQDQWGRQWCD-UHFFFAOYSA-N); 7-ethyl-9-amino-20(S)-camptothecin (XEZHQWJDQCESCS-QFIPXVFZSA-N); 7-methoxy-1-(3,4,5-trimethoxyphenyl)-4,5-dihydro-3H-benzo[e]indazole (HGVPEUJPOPWOGE-UHFFFAOYSA-N); 7-methoxyisoindolo[1,2-a]quinoxalin-4(5H)one (MKSBPJHYGYRHHG-UHFFFAOYSA-N); 702675-74-9 (VAVXGGRQQJZYBL-UHFFFAOYSA-N); 70844-09-6 (GDIORRHLBKYQCZ-PYBHCKQVSA-N); 73113-92-5 (FCYRUGFDQOKETE-AXBADRFBSA-N); 7356-74-3 (QKPVNWPUSANAKZ-UHFFFAOYSA-M); 7400-49-9 (ZHADVXQBQNXURV-UHFFFAOYSA-M); 741713-40-6 (JRNJNYBQQYBCLE-UHFFFAOYSA-N); 74296-42-7 (CCBHSPUYWZHDFX-UHFFFAOYSA-N); 761437-28-9 (UYJNQQDJUOUFQJ-UHFFFAOYSA-N); 761439-42-3 (QQWUGDVOUVUTOY-UHFFFAOYSA-N); 7770-39-0 (JTNAIYBZLQUATM-UHFFFAOYSA-M); 7H-Pyrrolo[2, 4-amino-N-hydroxy-7-beta-D-ribofuranosyl-(UJSNJRHHJZOXPZ-UHFFFAOYSA-N); 7H-Pyrrolo[3,4-diamine, 7-[(3-nitrophenyl)methyl]-, monoacetate (UFFKDOLCUFDMQB-UHFFFAOYSA-N); 8-Anilinoquinoline-5,6-dione (AMWHXDZWCAJAAT-UHFFFAOYSA-N); 8-Azaguanine (LPXQRXLUHJKZIE-UHFFFAOYSA-N); 8-Chloroadenosine (MHDPPLULTMGBSI-UUOKFMHZSA-N); 8-Hydroxy-2-methylquinazolin-4(3H)-one (YJDAOHJWLUNFLX-UHFFFAOYSA-N); 8-Hydroxyquinoline (MCJGNVYPOGVAJF-UHFFFAOYSA-N); 8-Hydroxyquinoline salicylate (GOEKBPHGLUYFOX-UHFFFAOYSA-N); 8-Hydroxyquinolinium hydrogen sulfate (MRUMAIRJPMUAPZ-UHFFFAOYSA-N); 8-alpha-Hydroxyverrucarin A (KLQXPYFNSWYRBB-YDFGWWAZSA-N); 828911-76-8 (QUOCIDQIFWYHLB-QHKWOANTSA-N); 867331-82-6 (JMGXJHWTVBGOKG-UHFFFAOYSA-N); 867334-05-2 (AQSSMEORRLJZLU-UHFFFAOYSA-N); 869288-64-2 (HESLKTSGTIBHJU-UHFFFAOYSA-N); 875787-07-8 (KYWWJENKIMRJBI-UHFFFAOYSA-N); 878692-44-5 (MOVCZXSLLYVASW-UHFFFAOYSA-N); 882531-87-5 (UOVCGJXDGOGOCZ-UHFFFAOYSA-N); 891494-63-6 (GMIZZEXBPRLVIV-SECBINFHSA-N); 8B-Hydroxy-9B,10B-epoxyverrucarin A (JATSIAAIWIOXMZ-YDFGWWAZSA-N); 9, bis(4,5-dihydro-1H-imidazol-2-ylhydrazone), dihydrochloride (HBSXKBIYGYFNRF-JMLRMIEWSA-N); 9,10-Anthracenedione, 1-hydroxy-4-((2-((2-hydroxyethyl)amino)ethyl)amino)-(ODQHMLBNJHJVIM-UHFFFAOYSA-N); 9,10-Phenanthrenequinone (YYVYAPXYZVYDHN-UHFFFAOYSA-N); 9-((2-(Dimethylamino)ethyl)amino)-1-(hydroxy(oxido)amino)-7-methoxyacridine (FGQDRDARUWHKTA-UHFFFAOYSA-N); 9-((3-(Hexylamino)propyl)amino)-1-(hydroxy(oxido)amino)-7-methoxyacridine (ULFUFZLCZTZFOI-UHFFFAOYSA-N); 9-(1,3-Dioxan-2-yl)-5,6,11-trimethylpyrido[4,3-b]carbazole (ONXKAECAHOAMFB-UHFFFAOYSA-N); 9-(3,5-Dimethoxyphenyl)-6-methoxy-4,9-dihydrofuro[3,4-b]quinolin-1(3H)-one (LGJCFMXXNMRJEO-UHFFFAOYSA-N); 9-(4-Cyclopropylpiperazin-1-yl)pyrido[3,2-b]phenazine-5,12-dione (BIHXLYFRQRWSPT-UHFFFAOYSA-N); 9-Acridinamine, N-(3-hydroxypropyl)-7-methoxy-1-nitro-(LDRJZRDGRTXWOE-UHFFFAOYSA-N); 9-Acridinamine, N-[2-[(2-hydroxyethyl)amino]ethyl]-7-methoxy-1-nitro-(JZPIJWMIMBFFJU-UHFFFAOYSA-N); 9-Acridinamine, N-[2-[bis(2-hydroxyethyl)amino]ethyl]-7-methoxy-1-nitro-(FSFCSVVEPROIAI-UHFFFAOYSA-N); 9-Acridinamine, dihydrochloride (NCPACVQCQSZHJP-UHFFFAOYSA-N); 9-Amino-5-imino-5H-benzo[a]phenoxazine acetate salt (ORIKHDNZZCTIAD-UHFFFAOYSA-N); 9-Amino-8-(hydroxymethyl)-2,3-dihydropyrrolo[2,3-g][1,4]benzothiazin-5-ol (KGENHOSDJOUGRC-UHFFFAOYSA-N); 9-Aminoacridine (XJGFWWJLMVZSIG-UHFFFAOYSA-N); 9-Aminocamptothecin (FUXVKZWTXQUGMW-FQEVSTJZSA-N); 9-Chloro-10,11-methylenedioxycamptothecin (AQSRKNJFNKOMDG-NRFANRHFSA-N); 9-Chloro-9-(4-methoxyphenyl)-10-phenyl-9,10-dihydroacridine (JFGXQMZJWOMCOV-UHFFFAOYSA-N); 9-Chloronaphtho[1′,2′:4,5]imidazo[1,2-a]pyridine-5,6-dione (VZYGUWNBWRRBMX-UHFFFAOYSA-N); 9-Deoxy-sibiromycin (FLYYWBJOHHJDAU-USXWEJKASA-N); 9-Methoxy-2,6-dimethylellipticinium iodide (FCYDLHSTLJWDAF-UHFFFAOYSA-M); 9-Methoxycamptothecin (XVMZDZFTCKLZTF-NRFANRHFSA-N); 9-Methoxyellipticine (BKRMCDAOAQWNTG-UHFFFAOYSA-N); 9-Methylnaphtho[1′,2′:4,5]imidazo[1,2-a]pyridine-5,6-dione (CKBXCZFOKKJSQY-UHFFFAOYSA-N); 9-Phenyl-6,9-dihydro[1,3]dioxolo[4,5-g]furo[3,4-b]quinolin-8(5H)-one (BDMYAZQPJOCOKN-UHFFFAOYSA-N); 9-[(6-bromo-1,3-benzodioxol-5-yl)methyl]-6-N-phenylpurine-2,6-diamine (MPXWFZINXMCPSU-UHFFFAOYSA-N); 9-[3-(2-Nitroamidazol-1-yl)propyl]amino-1-nitroacridine·HCl (XEGWFTABWZJOJE-UHFFFAOYSA-N); 9-[3-[N-(2-N,N-Dimethylamino)ethyl]aminopropyl]amino-1-nitroacridine·3HCl (XFGYCQUGYZLJMZ-UHFFFAOYSA-N); 9-methoxy-2,5,11-trimethyl-6H-pyrido[4,3-b]carbazolium iodide (UMHIZUSDRRRZIC-UHFFFAOYSA-N); 90914-48-0 (PVJKNKLXEAXWCF-UHFFFAOYSA-N); 911417-87-3 (GKHIVNAUVKXIIY-UHFFFAOYSA-N); 919486-40-1 (OZRMEKAUZBKTTC-UHFFFAOYSA-N); 936091-15-5 (MMXZBJJTHQWMEN-UHFFFAOYSA-N); 937174-76-0 (KGPGFQWBCSZGEL-ZDUSSCGKSA-N); 937270-47-8 (VXBAJLGYBMTJCY-IHWYPQMZSA-N); 940929-33-9 (MLMZVWABFOLFGV-LNLSOMNWSA-N); 946128-88-7 (LMMJFBMMJUMSJS-UHFFFAOYSA-N); 947303-87-9 (XKPJTOHUPQWSOJ-HSZRJFAPSA-N); 9H-Carbazole, 1,8-dichloro-3,6-dinitro-(YNBOZNTWWGDDLF-UHFFFAOYSA-N); 9H-Purine,8-diamino-9-beta-D-ribofuranosyl-(DVGWFQILDUEEGX-UHFFFAOYSA-N); A-443654 (YWTBGJGMTBHQTM-IBGZPJMESA-N); A-674563 (BPNUQXPIQBZCMR-IBGZPJMESA-N); ABBV-744 (OEDSFMUSNZDJFD-UHFFFAOYSA-N); ACRIDINE, 9-(p-DIMETHYLAMINOANILINO)-(WBERNRVMJQYKKR-UHFFFAOYSA-N); AG-2037; AG 2037; AG2037 (QXOPTIPQEVJERB-UHFFFAOYSA-N); AG14361 (SEKJSSBJKFLZIT-UHFFFAOYSA-N); AGN 195183 (PNAWUIKCVQSLFG-UHFFFAOYSA-N); AM580 (SZWKGOZKRMMLAJ-UHFFFAOYSA-N); AMG-208 (HEAIZQNMNCHNFD-UHFFFAOYSA-N); AMG-232 (DRLCSJFKKILATL-YWCVFVGNSA-N); AMG-458 (GLBZSOQDAOLMGC-UHFFFAOYSA-N); AMG-47a (DVRSTRMZTAPMKO-UHFFFAOYSA-N); AMG-51 (ONSZEUCTVQJHOZ-UHFFFAOYSA-N); AMG-517 (YUTIXVXZQIQWGY-UHFFFAOYSA-N); AMG-900 (IVUGFMLRJOCGAS-UHFFFAOYSA-N); ARQ 531 (JSFCZQSJQXFJDS-QAPCUYQASA-N); ARQ 621 (UPJSUQWHUVLLNW-XMMPIXPASA-N); ARRY-520 R-Enantiomer (LLXISKGBWFTGEI-UHFFFAOYSA-N); ASP3026 (MGGBYMDAPCCKCT-UHFFFAOYSA-N); ASP5878 (VDZZYOJYLLNBTD-UHFFFAOYSA-N); AST 487 (ODPGGGTTYSGTGO-UHFFFAOYSA-N); ASTX660 (YCXOHEXZVKOGEV-DNRQZRRGSA-N); AT9283 (LOLPPWBBNUVNQZ-UHFFFAOYSA-N); AV-412 free base (ZAJXXUDARPGGOC-UHFFFAOYSA-N); AVN-944 (GYCPCOJTCINIFZ-OXJNMPFZSA-N); AZ-960 (SUNXHXDJOIXABJ-NSHDSACASA-N); AZD-5153 (RSMYFSPOTCDHHJ-GOSISDBHSA-N); AZD-7648 (XISVSTPEXYIKJL-UHFFFAOYSA-N); AZD0156 (AOTRIQLYUAFVSC-UHFFFAOYSA-N); AZD1208 (MCUJKPPARUPFJM-UWCCDQBKSA-N); AZD1480 (PDOQBOJDRPLBQU-QMMMGPOBSA-N); AZD2858 (FHCSBLWRGCOVPT-UHFFFAOYSA-N); AZD3514 (JMEYDSHPKCSIJC-UHFFFAOYSA-N); AZD3759 (MXDSJQHFFDGFDK-CYBMUJFWSA-N); AZD3965 (PRNXOFBDXNTIFG-FQEVSTJZSA-N); AZD4573 free base (AVIWDYSJSPOOAR-LSDHHAIUSA-N); AZD7545 (DTDZLJHKVNTQGZ-GOSISDBHSA-N); AZD8055 (KVLFRAWTRWDEDF-IRXDYDNUSA-N); AZD8186 (LMJFJIDLEAWOQJ-CQSZACIVSA-N); AZD8330 (RWEVIPRMPFNTLO-UHFFFAOYSA-N); AZD8835 (ZGRDYKFVDCFJCZ-UHFFFAOYSA-N); AZD9496 (DFBDRVGWBHBJNR-BBNFHIFMSA-N); Abacavir (MCGSCOLBFJQGHM-SCZZXKLOSA-N); Abemaciclib (UZWDCWONPYILKI-UHFFFAOYSA-N); Abexinostat (MAUCONCHVWBMHK-UHFFFAOYSA-N); Abiraterone (GZOSMCIZMLWJML-VJLLXTKPSA-N); Abiraterone acetate (UVIQSJCZCSLXRZ-UBUQANBQSA-N); Abivertinib (UOFYSRZSLXWIQB-UHFFFAOYSA-N); Abt-737 (HPLNQCPCUACXLM-PGUFJCEWSA-N); Abt-751 (URCVCIZFVQDVPM-UHFFFAOYSA-N); Acalabrutinib (WDENQIQQYWYTPO-IBGZPJMESA-N); Acalisib (DOCINCLJNAXZQF-LBPRGKRZSA-N); Aceclofenac (MNIPYSSQXLZQLJ-UHFFFAOYSA-N); Aceglutamide (KSMRODHGGIIXDV-YFKPBYRVSA-N); Acelarin (NHTKGYOMICWFQZ-KKQYNPQSSA-N); Acemetacin (FSQKKOOTNAMONP-UHFFFAOYSA-N); Acetaminophen (RZVAJINKPMORJF-UHFFFAOYSA-N); Acetazolamide (BZKPWHYZMXOIDC-UHFFFAOYSA-N); Acetohydroxamic acid (RRUDCFGSUDOHDG-UHFFFAOYSA-N); Acetomenaphthone (RYWSYCQQUDFMAU-UHFFFAOYSA-N); Acetophenazine maleate (NUKVZKPNSKJGBK-SPIKMXEPSA-N); Acetoxy phenylmercury (XEBWQGVWTUSTLN-UHFFFAOYSA-M); Acetyloxycycloheximide (UFDHNJJHPSGMFX-UHFFFAOYSA-N); Acid Red 94 (UWBXIFCTIZXXLS-UHFFFAOYSA-L); Acitretin (IHUNBGSDBOWDMA-AQFIFDHZSA-N); Acivicin (QAWIHIJWNYOLBE-OKKQSCSOSA-N); Aclacinomycin x (OHMUKIWRHXAZQS-UHFFFAOYSA-N); Aclarlubicin hel (KUSMIBXCRZTVML-GCYJRYRVSA-N); Aclarubicin (USZYSDMBJDPRIF-SVEJIMAYSA-N); Aclucinomycin a (USZYSDMBJDPRIF-XCKHQVGJSA-N); Acolbifene (DUYNJNWVGIWJRI-LJAQVGFWSA-N); Acriflavine (PEJLNXHANOHNSU-UHFFFAOYSA-N); Acrisorcin (YZODJQFXMFEJRM-UHFFFAOYSA-N); Acrivastine (PWACSDKDOHSSQD-IUTFFREVSA-N); Actb-1003 (GZPJCJKUZPUFAL-UHFFFAOYSA-N); Actinobolin (PQVQBAAWCOTEBG-NSVWQLETSA-N); Actinomycin D 3-phenyloxazinone (VGEQFUNTHQCTFN-UHFFFAOYSA-N); Actinomycin c (QCXJFISCRQIYID-UHFFFAOYSA-N); Actinomycin d (RJURFGZVJUQBHK-UHFFFAOYSA-N); Actinomycin vii (XLGPRYUFOGVPMM-UHFFFAOYSA-N); Acyclovir (MKUXAQIIEYXACX-UHFFFAOYSA-N); Adagrasib (PEMUGDMSUDYLHU-ZEQRLZLVSA-N); Adapalene (LZCDAPDGXCYOEH-UHFFFAOYSA-N); Adaphostin (YJZSUCFGHXQWDM-UHFFFAOYSA-N); Adavosertib (BKWJAKQVGHWELA-UHFFFAOYSA-N); Adefovir (SUPKOOSCJHTBAH-UHFFFAOYSA-N); Adefovir dipivoxil (WOZSCQDILHKSGG-UHFFFAOYSA-N); Adenosine (OIRDTQYFTABQOQ-KQYNXXCUSA-N); Adenosine monophosphate (UDMBCSSLTHHNCD-KQYNXXCUSA-N); Adomeglivant (FASLTMSUPQDLIB-MHZLTWQESA-N); Adriamycin 14-octanoate, hydrochloride (PCIHZBKBBKWVRO-WLMNPPFISA-N); Adriamycin benzoate (MIYPZEOVBCFNMU-JEVKMULKSA-N); Adriamycin nicotinate (DFTYWNVALAXKHG-PAMQHEOOSA-N); Adriblastin (MWWSFMDVAYGXBV-FGBJBKNOSA-N); Adx-47273 (VXQCCZHCFBHTTD-HNNXBMFYSA-N); Afatinib (ULXXDDBFHOBEHA-CWDCEQMOSA-N); Afatinib dimaleate (USNRYVNRPYXCSP-JUGPPOIOSA-N); Afimoxifene (TXUZVZSFRXZGTL-QPLCGJKRSA-N); Afuresertib (AFJRDFWMXUECEW-LBPRGKRZSA-N); Agelastatin a (MPASKXAEPUAMBS-WJOUQXRDSA-N); Agerafenib (DKNUPRMJNUQNHR-UHFFFAOYSA-N); Agn-PC-ONI92I (BYDVHCHTUDOXGJ-UHFFFAOYSA-N); Alanosine monosodium salt ion (GHMZJZVTLXTLOF-YSMBQZINSA-N) salts; Albacarcin v (OMDANBMKOUVKAG-UHFFFAOYSA-N); Albendazole (HXHWSAZORRCQMX-UHFFFAOYSA-N); Albendazole oxide (VXTGHWHFYNYFFV-UHFFFAOYSA-N); Aldoxorubicin (OBMJQRLIQQTJLR-USGQOSEYSA-N); Alectinib (KDGFLJKFZUIJMX-UHFFFAOYSA-N); Alfatradiol (VOXZDWNPVJITMN-SFFUCWETSA-N); Alisertib (ZLHFILGSQDJULK-UHFFFAOYSA-N); Alitretinoin (SHGAZHPCJJPHSC-ZVCIMWCZSA-N); Allethrin (ZCVAOQKBXKSDMS-UHFFFAOYSA-N); Allitinib (MVZGYPSXNDCANY-UHFFFAOYSA-N); Allopurinol (OFCNXPDARWKPPY-UHFFFAOYSA-N); Allylestrenol (ATXHVCQZZJYMCF-XUDSTZEESA-N); Almitrine mesylate (MRDBGMJEPGXQHJ-UHFFFAOYSA-N); Almorexant (DKMACHNQISHMDN-RPLLCQBOSA-N); Aloin (AFHJQYHRLPMKHU-WEZNYRQKSA-N); Alosetron hydrochloride (FNYQZOVOVDSGJH-UHFFFAOYSA-N); Alpelisib (STUWGJZDJHPWGZ-LBPRGKRZSA-N); Alpidem (JRTIDHTUMYMPRU-UHFFFAOYSA-N); Alprostadil (GMVPRGQOIOIIMI-DWKJAMRDSA-N); Alrestatin (GCUCIFQCGJIRNT-UHFFFAOYSA-N); Alrizomadlin (YJCZPJQGFSSFOL-MNZPCBJKSA-N); Alsterpaullone (OLUKILHGKRVDCT-UHFFFAOYSA-N); Altiratinib (GNNDEPIMDAZHRQ-UHFFFAOYSA-N); Altrenogest (VWAUPFMBXBWEQY-ANULTFPQSA-N); Altretamine (UUVWYPNAQBNQJQ-UHFFFAOYSA-N); Alverine citrate (RYHCACJBKCOBTJ-UHFFFAOYSA-N); Alvocidib (BIIVYFLTOXDAOV-YVEFUNNKSA-N); Amantadine (DKNWSYNQZKUICI-UHFFFAOYSA-N); Ambroxol (JBDGDEWWOUBZPM-UHFFFAOYSA-N); Ambroxol hydrochloride (QNVKOSLOVOTXKF-UHFFFAOYSA-N); Amcenestrant (KISZAGQTIXIVAR-VWLOTQADSA-N); Amcinonide (ILKJAFIWWBXGDU-MOGDOJJUSA-N); Ametantrone (FFGSXKJJVBXWCY-UHFFFAOYSA-N); Amg-337 (DWHXUGDWKAIASB-CQSZACIVSA-N); Amifostine (JKOQGQFVAUAYPM-UHFFFAOYSA-N); Amikacin hydrate (DTSOZYYWEZJFSS-XTHCGPPUSA-N); Amiloride (XSDQTOBWRPYKKA-UHFFFAOYSA-N); Aminocaproic acid (SLXKOJJOQWFEFD-UHFFFAOYSA-N); Aminoglutethimide (ROBVIMPUHSLWNV-UHFFFAOYSA-N); Aminopterin (TVZGACDUOSZQKY-LBPRGKRZSA-N); Amiodarone (IYIKLHRQXLHMJQ-UHFFFAOYSA-N); Amiodarone hydrochloride (ITPDYQOUSLNIHG-UHFFFAOYSA-N); Amiselimod (JVCPIJKPAKAIIP-UHFFFAOYSA-N); Amisulpride (NTJOBXMMWNYJFB-UHFFFAOYSA-N); Amitriptyline hydrochloride (KFYRPLNVJVHZGT-UHFFFAOYSA-N); Amlexanox (SGRYPYWGNKJSDL-UHFFFAOYSA-N); Amlodipine (HTIQEAQVCYTUBX-UHFFFAOYSA-N); Amlodipine besylate (ZPBWCRDSRKPIDG-UHFFFAOYSA-N); Amodiaquin dihydrochloride dihydrate (YVNAYSHNIILOJS-UHFFFAOYSA-N); Amonafide (UPALIKSFLSVKIS-UHFFFAOYSA-N); Amorolfine (MQHLMHIZUIDKOO-AYHJJNSGSA-N); Amoxapine (QWGDMFLQWFTERH-UHFFFAOYSA-N); Amphethinile (LHNIUFUSFGYJEO-UHFFFAOYSA-N); Amphotericin b (APKFDSVGJQXUKY-INPOYWNPSA-N); Amrubicin (VJZITPJGSQKZMX-XDPRQOKASA-N); Amsacrine (XCPGHVQEEXUHNC-UHFFFAOYSA-N); Amsacrine hydrochloride (WDISRLXRMMTXEV-UHFFFAOYSA-N); Amsacrine mesylate (KIDGPIWSXHBPDH-UHFFFAOYSA-N); Amsalog (MWXDBWAFWBSGFA-UHFFFAOYSA-N); Amuvatinib (FOFDIMHVKGYHRU-UHFFFAOYSA-N); Amygdalin (XUCIJNAGGSZNQT-JHSLDZJXSA-N); Anacetrapib (MZZLGJHLQGUVPN-HAWMADMCSA-N); Anagrelide (OTBXOEAOVRKTNQ-UHFFFAOYSA-N); Anagrelide hydrochloride (TVWRQCIPWUCNMI-UHFFFAOYSA-N); Anastrozole (YBBLVLTVTVSKRW-UHFFFAOYSA-N); Ancitabine hydrochloride (KZOWNALBTMILAP-JBMRGDGGSA-N); Andarine (YVXVTLGIDOACBJ-SFHVURJKSA-N); Andrographolide (BOJKULTULYSRAS-OTESTREVSA-N); Androstanolone (NVKAWKQGWWIWPM-ABEVXSGRSA-N); Anethole trithione (KYLIZBIRMBGUOP-UHFFFAOYSA-N); Angelicin (XDROKJSWHURZGO-UHFFFAOYSA-N); Anguidine deriv 15-chloroacetylscirpene-3,4-diol (QPVVAWSGXBERRH-NCPLGYIASA-N); Aniracetam (ZXNRTKGTQJPIJK-UHFFFAOYSA-N); Anisindione (XRCFXMGQEVUZFC-UHFFFAOYSA-N); Anisomycin (YKJYKKNCCRKFSL-RDBSUJKOSA-N); Anisotropine methylbromide (QSFKGMJOKUZAJM-JXMYBXCISA-M); Anlotinib (KSMZEXLVHXZPEF-UHFFFAOYSA-N); Antazoline hydrochloride (SWKDMSRRIBZZAY-UHFFFAOYSA-N); Anthra(1,9-cd)pyrazol-6(2H)-one, 5-((3-aminopropyl)amino)-7,10-dihydroxy-2-(2-((2-hydroxyethyl)amino)ethyl)-, hydrochloride (AWVHFDOHKFRHKQ-UHFFFAOYSA-N); Anthra[1,9-cd]pyrazol-6(2H)-one der. (HWXPPHPMDNLIOG-UHFFFAOYSA-N); Anthracenedione diacetate (IAGMBSXKAGFBJT-UHFFFAOYSA-N); Anthralin (NUZWLKWWNNJHPT-UHFFFAOYSA-N); Anthramycin methyl ether (YRMCBQLZVBXOSJ-PCFSSPOYSA-N); Antibiotic AY 22989 (QFJCIRLUMZQUOT-ADLMHZFOSA-N); Antibiotic B 14437 (base) (OBZJZDHRXBKKTJ-UHFFFAOYSA-N); Antibiotic CC 1065 (UOWVMDUEMSNCAV-UHFFFAOYSA-N); Antibiotic K 252a (KOZFSFOOLUUIGY-SOLYNIJKSA-N); Antibiotic UK 63052 (VABRZWDEEDQLRD-UHFFFAOYSA-N); Antibiotic from streptomyces nogalater (KGTDRFCXGRULNK-QNQBSKBHSA-N); Antineoplastic-301477 (TUEYGBZZQFXBOS-FWBDKTPCSA-N); Antineoplastic-353527 ion (PGFSDGCUKKYRAQ-UHFFFAOYSA-M) salts; Antineoplastic-41390 (JCBAMEPRVWICDX-UHFFFAOYSA-N); Antineoplastic-615804 (NEOHHBPEWKUZMN-UHFFFAOYSA-N); Antineoplastic-616241 (ZCVLTIWFTLQSTM-UHFFFAOYSA-N); Antineoplastic-630680 (HDMUZLQWDPUHND-UHFFFAOYSA-N); Antineoplastic-641318 (ARYOZYVUZMGJCZ-XJVHOXIISA-N); Antineoplastic-641319 (VRIXQRXJYLPDBG-MHRQJLALSA-N); Antineoplastic-641454 (CJFQFKRQEHCVFU-CFYXSCKTSA-N); Antineoplastic-642198 (BVHNLAVZJZWVIX-YSMBQZINSA-N); Antineoplastic-642313 (FTBPECXVRQVZKY-UHFFFAOYSA-N); Antineoplastic-642314 (CWFCMSGWUJNTEL-UHFFFAOYSA-N); Antineoplastic-642315 (SGYHIXFRQZKXIO-UHFFFAOYSA-N); Antineoplastic-642316 (HSTBDXBIEVMIKQ-UHFFFAOYSA-N); Antineoplastic-642318 (JHMLEWBLPQMNBU-UHFFFAOYSA-N); Antineoplastic-642319 (OHTMCPHLARXTIF-UHFFFAOYSA-N); Antineoplastic-643812 ion (HRFFGGIUECGXRH-YSMBQZINSA-N) salts; Antineoplastic-645804 (SJNRPUAPZIOMGM-UHFFFAOYSA-N); Antineoplastic-645806 (NKRPYVUCGATPEE-UHFFFAOYSA-N); Antineoplastic-645807 (LOSSLHWHHZXNCI-UHFFFAOYSA-N); Antineoplastic-647246 (VJKRVUFIERGGGG-YSMBQZINSA-N); Antineoplastic-648771 (YLEQOBFRJJEUHL-UHFFFAOYSA-N); Antineoplastic-648772 (TUPBMYHLAAQFKG-UHFFFAOYSA-N); Antineoplastic-648782 (GWQSWLMEYHNEJW-UHFFFAOYSA-N); Antineoplastic-648783 (OFHATWPEJUVRKL-UHFFFAOYSA-N); Antineoplastic-648784 (PEHBKWRZINGKCK-UHFFFAOYSA-N); Antineoplastic-648786 (IBYUPDOODPVKHJ-UHFFFAOYSA-N); Antineoplastic-648787 (HFUAORNGBSYNJS-UHFFFAOYSA-N); Antineoplastic-648788 (SKBSXMPUTODDQR-UHFFFAOYSA-N); Antineoplastic-648789 (FAAULQKFYXONRY-UHFFFAOYSA-N); Antineoplastic-648790 (LQTSLJRIWRBARN-UHFFFAOYSA-N); Antineoplastic-672552 (FFQWUYZGOQOZFW-QHCPKHFHSA-N); Antineoplastic-690266 (DJFXWTPYURSOOJ-KSMMMRORSA-N); Antineoplastic-690267 (DJFXWTPYURSOOJ-ZVOCJMRISA-N); Antineoplastic-690269 (FHSOZQXNDQCQER-FATZIPQQSA-N); Antineoplastic-92904 (YOLKXVIMUSPZJK-UHFFFAOYSA-N); Antineoplastic-92915 (DMJGMSVPSALYGT-UHFFFAOYSA-N); Antineoplastic-D630679 (HBQDLHJADJTNRK-UHFFFAOYSA-N); Antineoplastic-D647752 (CFCVIJYLRRCBFF-WCYRVBQBSA-N); Antineoplastic-D647753 (HUSNEEVUDJQTBT-SUMUEXJXSA-N); Antineoplastic-D648114 (JXOGZJFJYDAGNR-UHFFFAOYSA-N); Antineoplastic-D656598 (BRAHPGVQCXWLHP-QQXYBPILSA-N); Apalutamide (HJBWBFZLDZWPHF-UHFFFAOYSA-N); Apaziquone (MXPOCMVWFLDDLZ-NSCUHMNNSA-N); Apigenin (KZNIFHPLKGYRTM-UHFFFAOYSA-N); Apitolisib (YOVVNQKCSKSHKT-HNNXBMFYSA-N); Apixaban (QNZCBYKSOIHPEH-UHFFFAOYSA-N); Apomorphine (VMWNQDUVQKEIOC-CYBMUJFWSA-N); Aprepitant (ATALOFNDEOCMKK-OITMNORJSA-N); Aranidipine (NCUCGYYHUFIYNU-UHFFFAOYSA-N); Arbidol (KCFYEAOKVJSACF-UHFFFAOYSA-N); Arctigenin (NQWVSMVXKMHKTF-JKSUJKDBSA-N); Argatroban (KXNPVXPOPUZYGB-IOVMHBDKSA-N); Aripiprazole (CEUORZQYGODEFX-UHFFFAOYSA-N); Aristolochic acid (BBFQZRXNYIEMAW-UHFFFAOYSA-N); Arlidin (CLJHABUMMDMAFA-UHFFFAOYSA-N); Arsenic trioxide (QTLQKAJBUDWPIB-UHFFFAOYSA-N); Artemether (SXYIRMFQILZOAM-HVNFFKDJSA-N); Artemisinin (BLUAFEHZUWYNDE-NNWCWBAJSA-N); Artesunic acid (FIHJKUPKCHIPAT-AHIGJZGOSA-N); Asciminib (VOVZXURTCKPRDQ-CQSZACIVSA-N); Asnuciclib (GAIOPWBQKZMUNO-UHFFFAOYSA-N); Aspirin (BSYNRYMUTXBXSQ-UHFFFAOYSA-N); Astemisinin (BLUAFEHZUWYNDE-DKGJTOOQSA-N); Astemizole (GXDALQBWZGODGZ-UHFFFAOYSA-N); Ataciguat (PQHLRGARXNPFCF-UHFFFAOYSA-N); Ataluren (OOUGLTULBSNHNF-UHFFFAOYSA-N); Atamparib (UQZCQKXJAXKZQH-LBPRGKRZSA-N); Atazanavir (AXRYRYVKAWYZBR-GASGPIRDSA-N); Atipamezole (HSWPZIDYAHLZDD-UHFFFAOYSA-N); Atipamezole hydrochloride (PCCVCJAQMHDWJY-UHFFFAOYSA-N); Atiprimod (SERHTTSLBVGRBY-UHFFFAOYSA-N); Atomoxetine hydrochloride (LUCXVPAZUDVVBT-UNTBIKODSA-N); Atopaxar (QWKAUGRRIXBIPO-UHFFFAOYSA-N); Atorvastatin (XUKUURHRXDUEBC-KAYWLYCHSA-N); Atorvastatin calcium (FQCKMBLVYCEXJB-MNSAWQCASA-L); Atovaquone (BSJMWHQBCZFXBR-UHFFFAOYSA-N); Atreleuton (MMSNEKOTSJRTRI-LLVKDONJSA-N); Atropine (RKUNBYITZUJHSG-PJPHBNEVSA-N); Avadomide (RSNPAKAFCAAMBH-UHFFFAOYSA-N); Avagacestat (XEAOPVUAMONVLA-QGZVFWFLSA-N); Avasimibe (PTQXTEKSNBVPQJ-UHFFFAOYSA-N); Avobenzone (XNEFYCZVKIDDMS-UHFFFAOYSA-N); Axitinib (RITAVMQDGBJQJZ-FMIVXFBMSA-N); Azacitidine (NMUSYJAQQFHJEW-KVTDHHQDSA-N); Azaperone (XTKDAFGWCDAMPY-UHFFFAOYSA-N); Azaribine (QQOBRRFOVWGIMD-OJAKKHQRSA-N); Azasetron (WUKZPHOXUVCQOR-UHFFFAOYSA-N); Azatadine (SEBMTIQKRHYNIT-UHFFFAOYSA-N); Azathioprine (LMEKQMALGUDUQG-UHFFFAOYSA-N); Azd-1152hqpa (QYZOGCMHVIGURT-UHFFFAOYSA-N); Azd-4877 (SMFXSYMLJDHGIE-UHFFFAOYSA-N); Azd4547 (VRQMAABPASPXMW-HDICACEKSA-N); Azelaic acid (BDJRBEYXGGNYIS-UHFFFAOYSA-N); Azelastine (MBUVEWMHONZEQD-UHFFFAOYSA-N); Azelastine hydrochloride (YEJAJYAHJQIWNU-UHFFFAOYSA-N); Azelnidipine (ZKFQEACEUNWPMT-UHFFFAOYSA-N); Azithromycin (MQTOSJVFKKJCRP-BICOPXKESA-N); BAY 87-2243 (CDJNNOJINJAXPV-UHFFFAOYSA-N); BAY-1436032 (RNMAUIMMNAHKQR-QFBILLFUSA-N); BAY-2402234 (KNVJMHHAXCPZHF-JTQLQIEISA-N); BAY-876 (BKLJDIJJOOQUFG-UHFFFAOYSA-N); BAY1125976 (JBGYKRAZYDNCNV-UHFFFAOYSA-N); BDBM6867 (IEWLQUDDCQIINQ-UHFFFAOYSA-N); BGT-226 free base (BMMXYEBLEBULND-UHFFFAOYSA-N); BI 2536 (XQVVPGYIWAGRNI-JOCHJYFZSA-N); BI-3406 (XVFDNRYZXDHTHT-PXAZEXFGSA-N); BI-847325 (OCUQMWSIGPQEMX-UHFFFAOYSA-N); BLU9931 (TXEBNKKOLVBTFK-UHFFFAOYSA-N); BLZ945 (ADZBMFGQQWPHMJ-RHSMWYFYSA-N); BMS-214662 (OLCWFLWEHWLBTO-HSZRJFAPSA-N); BMS-536924 (ZWVZORIKUNOTCS-OAQYLSRUSA-N); BMS-582949 (GDTQLZHHDRRBEB-UHFFFAOYSA-N); BMS-599626 (LUJZZYWHBDHDQX-QFIPXVFZSA-N); BMS-690514 (CSGQVNMSRKWUSH-IAGOWNOFSA-N); BMS-777607 (VNBRGSXVFBYQNN-UHFFFAOYSA-N); BMS-833923 (KLRRGBHZCJLIEL-UHFFFAOYSA-N); BMS-986020 (GQBRZBHEPUQRPL-LJQANCHMSA-N); BMS-986158 (KGERZPVQIRYWRK-GDLZYMKVSA-N); BNC105p (VVIPLWCZZYERCA-UHFFFAOYSA-L); BTZO43 Racemate (GTUIRORNXIOHQR-UHFFFAOYSA-N); BX-912 (DMMILYKXNCVKOJ-UHFFFAOYSA-N); Baccharin (DGBITFNXKQHKLI-LCTKYAGXSA-N); Baccharin B4 (ZGOCMMMDEQOCDU-APBVVRNWSA-N); Baccharinoid A-1 (busam) (XGOQVDSNQJJHTN-CDJQDVQCSA-N); Baccharis principle B-I (B800157F246 and K380) (PYYBXMVTBWYBDY-QOOAFILCSA-N); Bactobolin (RBCHRRIVFAIGFI-RGBMRXMBSA-N); Bafetinib (ZGBAJMQHJDFTQJ-DEOSSOPVSA-N); Baicalein (FXNFHKRTJBSTCS-UHFFFAOYSA-N); Balofloxacin (MGQLHRYJBWGORO-UHFFFAOYSA-N); Balsalazide (IPOKCKJONYRRHP-UHFFFAOYSA-N); Barasertib (GBJVVSCPOBPEIT-UHFFFAOYSA-N); Bardoxolone (TXGZJQLMVSIZEI-UQMAOPSPSA-N); Bardoxolone methyl (WPTTVJLTNAWYAO-KPOXMGGZSA-N); Baricitinib (XUZMWHLSFXCVMG-UHFFFAOYSA-N); Basic blue 7 (ROVRRJSRRSGUOL-UHFFFAOYSA-N); Basimglurant (UPZWINBEAHDTLA-UHFFFAOYSA-N); Batabulin (ROZCIVXTLACYNY-UHFFFAOYSA-N); Batimastat (XFILPEOLDIKJHX-QYZOEREBSA-N); Bay 41-2272 (ATOAHNRJAXSBOR-UHFFFAOYSA-N); Bazedoxifene (UCJGJABZCDBEDK-UHFFFAOYSA-N); Bazedoxifene acetate (OMZAMQFQZMUNTP-UHFFFAOYSA-N); Bbi-503(BB-503) (QDWKGEFGLQMDAM-JJIBRWJFSA-N); Bctc (ROGUAPYLUCHQGK-UHFFFAOYSA-N); Beclomethasone (NBMKJKDGKREAPL-DVTGEIKXSA-N); Beclomethasone dipropionate (KUVIULQEHSCUHY-XYWKZLDCSA-N); Bedaquiline (QUIJNHUBAXPXFS-XLJNKUFUSA-N); Belinostat (NCNRHFGMJRPRSK-MDZDMXLPSA-N); Belizatinib (WSTUJEXAPHIEIM-UHFFFAOYSA-N); Belnacasan (SJDDOCKBXFJEJB-MOKWFATOSA-N); Belotecan (LNHWXBUNXOXMRL-VWLOTQADSA-N); Belzutifan (LOMMPXLFBTZENJ-ZACQAIPSSA-N); Bemcentinib (KXMZDGSRSGHMMK-VWLOTQADSA-N); Benazepril (XPCFTKFZXHTYIP-PMACEKPBSA-N); Benazepril hydrochloride (VPSRQEHTHIMDQM-FKLPMGAJSA-N); Bendamustine (YTKUWDBFDASYHO-UHFFFAOYSA-N); Bendamustine hydrochloride (ZHSKUOZOLHMKEA-UHFFFAOYSA-N); Bengamide a (NKASPYGDXVCCTA-NRLSUAAVSA-N); Benidipine (QZVNQOLPLYWLHQ-ZEQKJWHPSA-N); Benidipine hydrochloride (KILKDKRQBYMKQX-MIPPOABVSA-N); Benperidol (FEBOTPHFXYHVPL-UHFFFAOYSA-N); Benserazide (BNQDCRGUHNALGH-UHFFFAOYSA-N); Benserazide hydrochloride (ULFCBIUXQQYDEI-UHFFFAOYSA-N); Bentamapimod (XCPPIJCBCWUBNT-UHFFFAOYSA-N); Benzamide, 4-(1-aminoethyl)-N-1H-pyrrolo(2,3-b)pyridin-4-yl-, (R)-(JTVBXQAYBIJXRP-SNVBAGLBSA-N); Benzamidine hydrochloride (LZCZIHQBSCVGRD-UHFFFAOYSA-N); Benzbromarone (WHQCHUCQKNIQEC-UHFFFAOYSA-N); Benzenecarboximidamide, 4-methyl-N′-phenyl-N-(triphenylphosphoranylidene)-(GRKPHEIHPBJWHB-UHFFFAOYSA-N); Benzethonium chloride (UREZNYTWGJKWBI-UHFFFAOYSA-M); Benzethonium ion (SIYLLGKDQZGJHK-UHFFFAOYSA-N) salts; Benzimidazole thiosemicarbazone derivative (JHYAWTAJVUIOAY-FBHDLOMBSA-N); Benziodarone (CZCHIEJNWPNBDE-UHFFFAOYSA-N); Benznidazole (CULUWZNBISUWAS-UHFFFAOYSA-N); Benzo[1,4-b′]dithiophene-4,8-dione, 2,5-bis(1-acetoxyethyl)-(JFEMGTTXNHRUOR-UHFFFAOYSA-N); Benzo[1,4-b′]dithiophene-4,8-dione, 2-(1-hydroxyethyl)-(ICOJWFLYZGAGHZ-UHFFFAOYSA-N); Benzo[1,4-b′]dithiophene-4,8-dione, 2-acetyl-(TYNBBESGBCIKHW-UHFFFAOYSA-N); Benzo[1,5-b′]dithiophene-4,8-dione, 2,7-bis(1-hydroxyethyl)-(UCYRPWDOWLXJHZ-UHFFFAOYSA-N); Benzo[1,5-b′]dithiophene-4,8-dione, 2-(1-hydroxyethyl)-(HLSTYWBADAGQLR-UHFFFAOYSA-N); Benzocaine (BLFLLBZGZJTVJG-UHFFFAOYSA-N); Benzofuran, monohydrochloride (WEKHTFMDMBQDAV-UHFFFAOYSA-N); Benzofurazan, 4-(4-methyl-1-piperazinyl)-7-nitro-, 3-oxide (MWFZDJLPWDCQIL-UHFFFAOYSA-N); Benzoylstaurosporine (BMGQWWVMWDBQGC-PZFYLCKJSA-N); Benztropine (GIJXKZJWITVLHI-YOFSQIOKSA-N); Benztropine mesylate (CPFJLLXFNPCTDW-IIPFOPBBSA-N); Benzydamine hydrochloride (HNNIWKQLJSNAEQ-UHFFFAOYSA-N); Benzyl 2-[1-(2-pyridinyl)ethylidene]hydrazinecarbodithioate (GAPPGYVPJUGIHP-SFQUDFHCSA-N); Benzyl 4-hydroxybenzoate (MOZDKDIOPSPTBH-UHFFFAOYSA-N); Benzyl isothiocyanate (MDKCFLQDBWCQCV-UHFFFAOYSA-N); Benzyldimethyl(tridecyl)ammonium chloride (XIWFQDBQMCDYJT-UHFFFAOYSA-M); Bepridil (UIEATEWHFDRYRU-UHFFFAOYSA-N); Berbamine (DFOCUWZXJBAUSQ-URLMMPGGSA-N); Berberine chloride (VKJGBAJNNALVAV-UHFFFAOYSA-M); Berberine ion (YBHILYKTIRIUTE-UHFFFAOYSA-N) salts; Bergapten (BGEBZHIAGXMEMV-UHFFFAOYSA-N); Berzosertib (JZCWLJDSIRUGIN-UHFFFAOYSA-N); Bet-IN-4 (YVGWSVHZXWFLIT-UHFFFAOYSA-N); Betamethasone dipropionate (CIWBQSYVNNPZIQ-XYWKZLDCSA-N); Betamethasone valerate (SNHRLVCMMWUAJD-SUYDQAKGSA-N); Betaxolol (NWIUTZDMDHAVTP-UHFFFAOYSA-N); Bethanechol chloride (XXRMYXBSBOVVBH-UHFFFAOYSA-N); Betulinic acid (QGJZLNKBHJESQX-FZFNOLFKSA-N); Bevantolol hydrochloride (FJTKCFSPYUMXJB-UHFFFAOYSA-N); Bexarotene (NAVMQTYZDKMPEU-UHFFFAOYSA-N); Biantrazole (GDNYMYIKZYXFET-UHFFFAOYSA-N); Bicalutamide (LKJPYSCBVHEWIU-UHFFFAOYSA-N); Bifemelane (QSQQPMHPCBLLGX-UHFFFAOYSA-N); Bifonazole (OCAPBUJLXMYKEJ-UHFFFAOYSA-N); Bimiralisib (ADGGYDAFIHSYFI-UHFFFAOYSA-N); Bindarit (MTHORRSSURHQPZ-UHFFFAOYSA-N); Binimetinib (ACWZRVQXLIRSDF-UHFFFAOYSA-N); Biotin (YBJHBAHKTGYVGT-ZKWXMUAHSA-N); Biperiden hydrochloride (RDNLAULGBSQZMP-UHFFFAOYSA-N); Birabresib (GNMUEVRJHCWKTO-FQEVSTJZSA-N); Birinapant (PKWRMUKBEYJEIX-DXXQBUJASA-N); Bis(2-ethylhexyl) phthalate (BJQHLKABXJIVAM-UHFFFAOYSA-N); Bis(3-mesyloxypropyl)amine hydrochloride (YWCASUPWYFFUHE-UHFFFAOYSA-N); Bisacodyl (KHOITXIGCFIULA-UHFFFAOYSA-N); Bisantrene (NJSMWLQOCQIOPE-OCHFTUDZSA-N); Bisbenzimide (PRDFBSVERLRRMY-UHFFFAOYSA-N); Bisoprolol fumarate (VMDFASMUILANOL-WXXKFALUSA-N); Bisphenol a (IISBACLAFKSPIT-UHFFFAOYSA-N); Bithionol (JFIOVJDNOJYLKP-UHFFFAOYSA-N); Bitolterol (FZGVEKPRDOIXJY-UHFFFAOYSA-N); Bizelesin (FONKWHRXTPJODV-DNQXCXABSA-N); Blenoxane (tn) (bristol meyers) (WUIABRMSWOKTOF-NUAMMRKQSA-N); Blonanserin (XVGOZDAJGBALKS-UHFFFAOYSA-N); Bms-707035 (VNIWZCGZPBJWBI-UHFFFAOYSA-N); Bms-754807 (LQVXSNNAFNGRAH-QHCPKHFHSA-N); Bms-911543 (JCINBYQJBYJGDM-UHFFFAOYSA-N); Bms-986142 (ZRYMMWAJAFUANM-INIZCTEOSA-N); Boronic acid, L-(S) (BPXDSWBQAUJBST-ZEQRLZLVSA-N); Borrelidin (OJCKRNPLOZHAOU-SLNPHPKOSA-N); Bortezomib (GXJABQQUPOEUTA-RDJZCZTQSA-N); Boscalid (WYEMLYFITZORAB-UHFFFAOYSA-N); Bosutinib (UBPYILGKFZZVDX-UHFFFAOYSA-N); Bouvardia ternafolia (UTRRTPWLDQPVJH-UHFFFAOYSA-N); Bouvardin (TWOWSRSKGJSZHZ-UHFFFAOYSA-N); Bozitinib (QHXLXUIZUCJRKV-UHFFFAOYSA-N); Brefeldin a (KQNZDYYTLMIZCT-KQPMLPITSA-N); Brexpiprazole (ZKIAIYBUSXZPLP-UHFFFAOYSA-N); Briciclib (LXENKEWVEVKKGV-BQYQJAHWSA-N); Brigatinib (AILRADAXUVEEIR-UHFFFAOYSA-N); Brilanestrant (BURHGPHDEVGCEZ-KJGLQBJMSA-N); Brilliant green (NNBFNNNWANBMTI-UHFFFAOYSA-M); Brinzolamide (HCRKCZRJWPKOAR-JTQLQIEISA-N); Bristol A-649 (WURKLEUBADSHTC-UHFFFAOYSA-N); Brivanib (WCWUXEGQKLTGDX-LLVKDONJSA-N); Brivanib alaninate (LTEJRLHKIYCEOX-OCCSQVGLSA-N); Bromazine (NUNIWXHYABYXKF-UHFFFAOYSA-N); Bromhexine (OJGDCBLYJGHCIH-UHFFFAOYSA-N); Bromhexine hydrochloride (UCDKONUHZNTQPY-UHFFFAOYSA-N); Bromo(triphenyl)plumbane (ZJLFYKFRSBDOTE-UHFFFAOYSA-M); Bromocriptine (OZVBMTJYIDMWIL-AYFBDAFISA-N); Bromocryptine mesylate (NOJMTMIRQRDZMT-JGYCFGIMSA-N); Bromopride (GIYAQDDTCWHPPL-UHFFFAOYSA-N); Bromperidol (RKLNONIVDFXQRX-UHFFFAOYSA-N); Bronopol (LVDKZNITIUWNER-UHFFFAOYSA-N); Brotizolam (UMSGKTJDUHERQW-UHFFFAOYSA-N); Broxyquinoline (ZDASUJMDVPTNTF-UHFFFAOYSA-N); Bruceantin (IRQXZTBHNKVIRL-GOTQHHPNSA-N); Bryamycin (NSFFHOGKXHRQEW-DVRIZHICSA-N); Bucillamine (VUAFHZCUKUDDBC-BYPYZUCNSA-N); Bucindolol (FBMYKMYQHCBIGU-UHFFFAOYSA-N); Buclizine dihydrochloride (SDBHDSZKNVDKNU-UHFFFAOYSA-N); Bufexamac (MXJWRABVEGLYDG-UHFFFAOYSA-N); Bunamidine hydrochloride (PBYJDHRFPFHVKZ-UHFFFAOYSA-N); Buparlisib (CWHUFRVAEUJCEF-UHFFFAOYSA-N); Buparvaquone (NEGDTWQGGLJCTL-UHFFFAOYSA-N); Bupropion (SNPPWIUOZRMYNY-UHFFFAOYSA-N); Bupropion hydrochloride (HEYVINCGKDONRU-UHFFFAOYSA-N); Buspirone (QWCRAEMEVRGPNT-UHFFFAOYSA-N); Busulfan (COVZYZSDYWQREU-UHFFFAOYSA-N); Butacaine (HQFWVSGBVLEQGA-UHFFFAOYSA-N); Butenafine (ABJKWBDEJIDSJZ-UHFFFAOYSA-N); Butenafine hydrochloride (LJBSAUIFGPSHCN-UHFFFAOYSA-N); Buthionine sulfoximine (KJQFBVYMGADDTQ-UHFFFAOYSA-N); Butoconazole (SWLMUYACZKCSHZ-UHFFFAOYSA-N); Butoconazole nitrate (ZHPWRQIPPNZNML-UHFFFAOYSA-N); Butriptyline (ALELTFCQZDXAMQ-UHFFFAOYSA-N); Butyl 4-aminobenzoate (IUWVALYLNVXWKX-UHFFFAOYSA-N); Butylcycloheptylprodiginine hydrochloride (XHNIOIAAXICRGD-PNNXLEGYSA-N); C.I. Basic Blue 3 (IURGIPVDZKDLIX-UHFFFAOYSA-M); C.I. Direct blue 53 (ATNOAWAQFYGAOY-UHFFFAOYSA-J); C.I. Solvent Blue 3 (KMGIACBASLJNIL-UHFFFAOYSA-N); C188-9 (QDCJDYWGYVPBDO-UHFFFAOYSA-N); CB-5083 (RDALZZCKQFLGJP-UHFFFAOYSA-N); CBL-0137 free base (JKCSODVERGVDLT-UHFFFAOYSA-N); CC-401 (XDJCLCLBSGGNKS-UHFFFAOYSA-N); CC-90009 (PWBHUSLMHZLGRN-UHFFFAOYSA-N); CCT129202 (QYKHWEFPFAGNEV-UHFFFAOYSA-N); CCT245737 (YBYYWUUUGCNAHQ-LLVKDONJSA-N); CEP-40783 (FKCWHHYUMFGOPY-UHFFFAOYSA-N); CFI-400945 free base (DADASRPKWOGKCU-FVTQAUBDSA-N); CH5132799 (JEGHXKRHKHPBJD-UHFFFAOYSA-N); CH7057288 (DCGOHGQJHJXBGW-UHFFFAOYSA-N); CHEBI:26536 (SHGAZHPCJJPHSC-UHFFFAOYSA-N); CHEBI:63449 (JNDVEAXZWJIOKB-UHFFFAOYSA-N); CHEMBL1084450 (FDCXXPBBHPICRU-UHFFFAOYSA-N); CHEMBL1086839 (YGEIJPASRUSQQD-UHFFFAOYSA-N); CHEMBL1087218 (IQVQCEHRAJVTIU-UHFFFAOYSA-N); CHEMBL1090207 (DWTHWQFUKBSGGN-UHFFFAOYSA-N); CHEMBL1090208 (QSXIDUKAGCAOKB-UHFFFAOYSA-N); CHEMBL1162119 (NKUQPNQBBNQMFU-UHFFFAOYSA-N); CHEMBL1213120 (HFGDXJQGICLTIE-UHFFFAOYSA-N); CHEMBL1237054 (XDKMHMINQXATOR-RCFLUEAHSA-N); CHEMBL1240647 (PJJRQEJQCFPFNT-LNMUWQJVSA-N); CHEMBL1240849 (DPYUJKQDLJBHMB-CTLOQAHHSA-N); CHEMBL1242929 (JSGUCQKJFWSGJD-MACQFCPGSA-N); CHEMBL1243115 (KPHFCEHLXFRNMH-PGZQALPFSA-N); CHEMBL1289827 (OXXBZZURMWFYLR-UHFFFAOYSA-N); CHEMBL1290691 (KIBFTWWVUPGBMR-UHFFFAOYSA-N); CHEMBL141997 (SGUMXALIXOMJSH-UHFFFAOYSA-N); CHEMBL150607 (IAVQJNSRTDVUJY-UHFFFAOYSA-N); CHEMBL1773157 (HTGDFPPUIOQBQB-UHFFFAOYSA-N); CHEMBL1782990 (INMQHLIMMGRRJC-UHFFFAOYSA-N); CHEMBL18155 (NYQPLPNEESYGNO-UHFFFAOYSA-N); CHEMBL1822215 (QBXSFEQYLUYSMX-UHFFFAOYSA-N); CHEMBL1830546 (HVBSSJMEZFQFCH-PAXDZCIKSA-N); CHEMBL1830547 (IQQFQJCQWFEBBU-HPVOOMOESA-N); CHEMBL1914978 (ORIVTYHQODIOFJ-UHFFFAOYSA-N); CHEMBL1914979 (PUPBQXYQKQWRSV-UHFFFAOYSA-N); CHEMBL1914982 (KESFQAMWJBXFEV-UHFFFAOYSA-N); CHEMBL1914983 (DCPSZGAQQPCAER-UHFFFAOYSA-N); CHEMBL1915463 (YVHIIJAEZHQWKT-UHFFFAOYSA-N); CHEMBL1915466 (VCDZPEUTOVBEPL-UHFFFAOYSA-N); CHEMBL1917373 (TYHUBYKXPIXYJV-UHFFFAOYSA-N); CHEMBL1940227 (CQGIUFASDNXPRB-UHFFFAOYSA-N); CHEMBL1940712 (DFLLESXVHVLXDP-UHFFFAOYSA-N); CHEMBL1966211 (CYXNWGGLZBUIJH-BYCVLTJGSA-N); CHEMBL1972734 (IQYRZKUVJRVIFN-HYGYKECYSA-N); CHEMBL1973379 (SKGNMALTNGTDEC-UHFFFAOYSA-N); CHEMBL1974148 (QFEWFWSZRWJSHW-HPCMTVGLSA-N); CHEMBL1975013 (CMDXFGUFUFEHBG-UHFFFAOYSA-N); CHEMBL1977420 (NFHCKNWFZUFPRO-JXMOYSMYSA-N); CHEMBL1981552 (AWFYHUCONKUXOE-UHFFFAOYSA-N); CHEMBL1983566 (LOYDTENNTZZQJM-FJVZNZLHSA-N); CHEMBL1987989 (FZAZHWMIBYMTGM-UHFFFAOYSA-N); CHEMBL1988721 (RQQLRUCQWPARKY-UHFFFAOYSA-N); CHEMBL1993566 (HVEDUANUPUFIBT-KWCYCYFSSA-N); CHEMBL2002528 (GVQMDKCFILZNNK-UHFFFAOYSA-N); CHEMBL2010923 (SJGGWYVNZAEBPH-UHFFFAOYSA-N); CHEMBL2043350 (GAWSDOMFNFILEC-UHFFFAOYSA-N); CHEMBL2043351 (PZEXTWWIKNZVMJ-UHFFFAOYSA-N); CHEMBL2043353 (NOHJUDQLISYZGE-UHFFFAOYSA-N); CHEMBL2295982 (CIGSBCIJOLHRPQ-XWISFQRESA-N); CHEMBL2296453 (HICKHUWRQNHRDE-UHFFFAOYSA-N); CHEMBL2298717 (KZVARHNNGCOEGO-FVDSYPCUSA-N); CHEMBL2335995 (UKLRGEOCJOIZCS-UHFFFAOYSA-N); CHEMBL2335999 (QOMQBMRBVJIEGL-UHFFFAOYSA-N); CHEMBL2402343 (UAXNYZPENRXDKY-MAPQJVESSA-N); CHEMBL2402344 (XNYHWEVYDZHFQM-OMKKRTLHSA-N); CHEMBL2402345 (WELPQYPQVLEGSI-MAPQJVESSA-N); CHEMBL2402347 (CSSYWQYQCALYND-SAADUKRKSA-N); CHEMBL2402348 (MJJBGCXYQKBMCD-QFAKEOTJSA-N); CHEMBL2402350 (XYHXWNUKNRJNQN-JDGPEGMASA-N); CHEMBL2402351 (BVKHRFLVYOCGMF-JDGPEGMASA-N); CHEMBL2402353 (BRRPMHYWWKCDRQ-HKNYPKJUSA-N); CHEMBL2402354 (YRNWYOCZYBQCDE-QFUPPCKTSA-N); CHEMBL2402355 (NNPOPIWCZIERSZ-DJLXOVMMSA-N); CHEMBL2402357 (QYMLLLAINXDCQI-XBYDYZNLSA-N); CHEMBL2402946 (UVKLAINVJNUJNX-UHFFFAOYSA-N); CHEMBL2402947 (NTFUBXCARKXYCA-UHFFFAOYSA-N); CHEMBL2403094 (MDEXMDFOZRJJIN-UHFFFAOYSA-N); CHEMBL2403096 (AJNPHCMZNHMLAQ-UHFFFAOYSA-N); CHEMBL2425968 (NAMOJMZBPJZNOD-CMDGGOBGSA-N); CHEMBL2436594 (OOTLBIRBZZTHQA-SZPBEECKSA-N); CHEMBL2443293 (IGBCYCNBGRBOKC-UHFFFAOYSA-N); CHEMBL248541 (BZQFSNUNMJEMCG-POHAHGRESA-N); CHEMBL248742 (GFNMYAMJZGFQKK-POHAHGRESA-N); CHEMBL248921 (FCVBLEXFCIZQCO-YHYXMXQVSA-N); CHEMBL249129 (YHRYEMLGRQUXSK-YHYXMXQVSA-N); CHEMBL251563 (USJNTUCQFCQOLU-WDZFZDKYSA-N); CHEMBL256443 (IFVRGFMZYKIFQI-YUAYGMJFSA-N); CHEMBL3113122 (XSKZNHPFLZNNHA-UHFFFAOYSA-N); CHEMBL3116069 (BDOCUEXZFMDKTJ-UHFFFAOYSA-N); CHEMBL3133724 (NOHKQCXDGOUSDA-PZWLIILKSA-N); CHEMBL3221041 (PWWXJRATTSECRZ-SANMLTNESA-N); CHEMBL3289214 (IKBYBHBSZPXPIL-BHVANESWSA-N); CHEMBL3338351 (JVONZUTUVAVGPK-UHFFFAOYSA-N); CHEMBL3338352 (ZKKWNFUBKHRUMT-UHFFFAOYSA-N); CHEMBL3338355 (OMVBTQBLPPMCEI-UHFFFAOYSA-N); CHEMBL3338356 (GDCFXRPYEWWCSY-UHFFFAOYSA-N); CHEMBL3338358 (QZKWJWJZWYRWMR-UHFFFAOYSA-N); CHEMBL3338360 (LFLKZTIFAOFDNF-UHFFFAOYSA-N); CHEMBL3338364 (PPSSNNXVCWTZKK-UHFFFAOYSA-N); CHEMBL3338365 (CODMLZKFWBTQFC-UHFFFAOYSA-N); CHEMBL3338367 (TZIPAQBRULNFOF-UHFFFAOYSA-N); CHEMBL3338372 (KWTPYZVIVMRWIC-UHFFFAOYSA-N); CHEMBL3342354 (VJFKEBZMNSAOIO-UHFFFAOYSA-N); CHEMBL3393594 (MXPRZJWKEJGHJF-UHFFFAOYSA-N); CHEMBL3422309 (UYIUMIAGZFCLKR-FNORWQNLSA-N); CHEMBL3545817 (BPZOGWXXIUMIML-UHFFFAOYSA-N); CHEMBL3586095 (SBQNYWSGQZHIRT-YRPGVFIESA-N); CHEMBL3593247 (VEDPEVJAYWVUSJ-BNVDZZMOSA-N); CHEMBL3593406 (FZBLZWMNFPJGGN-SZPBEECKSA-N); CHEMBL3593408 (MYIDUKTYJBIPIN-RKLUHIBISA-N); CHEMBL3593410 (GENMLQRMGWMMBT-FXLQIIFISA-N); CHEMBL3593411 (YSQNJKVHVWRCPW-FXHDEUNJSA-N); CHEMBL3612779 (OQZKRMWOMVBGNO-UHFFFAOYSA-N); CHEMBL3634466 (PBCYNJBBMUYAIG-JNMFGIBUSA-N); CHEMBL3634470 (BVPUTZCUALPGIM-YQZUZBKMSA-N); CHEMBL363844 (JFLPFZZKAULZBF-UHFFFAOYSA-N); CHEMBL3763462 (INXOSBIKPYGOJK-UHFFFAOYSA-N); CHEMBL3763467 (FMGXTLBJBMKAAM-PDSXEYIOSA-N); CHEMBL3763597 (UWXUPGPYKSOJJG-UHFFFAOYSA-N); CHEMBL3763610 (NMHQRFSBLFQRMV-UHFFFAOYSA-N); CHEMBL3763868 (VYUYFJRNLYJOIS-UHFFFAOYSA-N); CHEMBL3764089 (ILEBIIJILXMXHG-QMMMGPOBSA-N); CHEMBL3764791 (LAJPBLOIENLNAS-JTQLQIEISA-N); CHEMBL3765332 (ARIPEJUFKRWZFM-QMMMGPOBSA-N); CHEMBL3765490 (NVWZHPZYXUQSKV-UHFFFAOYSA-N); CHEMBL3765705 (YFHMWOJCGJARKT-UHFFFAOYSA-N); CHEMBL3809043 (VEHGJNKIAVQNKN-UHFFFAOYSA-N); CHEMBL3809638 (PWMMZOGAWPJKEC-UHFFFAOYSA-N); CHEMBL3809775 (PJQUZTXDFLIJNA-UHFFFAOYSA-N); CHEMBL3893211 (LPOCXXICQMIVLE-ITPPLTJNSA-N); CHEMBL3990408 ion (QDUXPUPOBLWAQP-UHFFFAOYSA-N) salts; CHEMBL3991933 (JUUFLLKHNYWHKP-UHFFFAOYSA-N); CHEMBL399555 (FTBQFBRYZNXQNS-WDZFZDKYSA-N); CHEMBL4060364 (XMWMSWMZCQBTET-ACHIHNKUSA-N); CHEMBL4062769 (INJUSSRMORXZTG-UHFFFAOYSA-N); CHEMBL4063011 (IDNPTPWRYDBQMT-UHFFFAOYSA-N); CHEMBL4064072 (GBNSKQWRSBCCAG-UHFFFAOYSA-N); CHEMBL4069903 (PWLYAHAXYULARD-UHFFFAOYSA-N); CHEMBL4070873 (BNRFZTXOBLEOGA-UHFFFAOYSA-N); CHEMBL4072994 (PAEFTRUWDBYVEW-UHFFFAOYSA-N); CHEMBL4076362 (VRXVSWVQXIEINY-UHFFFAOYSA-N); CHEMBL4079210 (FRPPIQVXSWLPGY-UHFFFAOYSA-N); CHEMBL4079995 (HYHOVKFBRXRMNX-UHFFFAOYSA-N); CHEMBL4080584 (VPADSKSPZOJDAY-UHFFFAOYSA-N); CHEMBL4081000 (IDPVANAVAHLPSG-UHFFFAOYSA-N); CHEMBL4084447 (AJWWCEIFWTXJEW-UHFFFAOYSA-N); CHEMBL4085641 (VGOQHXKBHKPKGX-UHFFFAOYSA-N); CHEMBL4091846 (JBWPEZUVPGPKOS-UHFFFAOYSA-N); CHEMBL4092588 (OHFXFRJMJUJUJA-UHFFFAOYSA-N); CHEMBL4094373 (JSNXSAANPIBWQQ-UHFFFAOYSA-N); CHEMBL4099532 (AZIHKCQBIHZGQA-UHFFFAOYSA-N); CHEMBL4100222 (AVJRYXYYXOTLJT-UHFFFAOYSA-N); CHEMBL4100617 (XOJIAQZVODHKEX-UHFFFAOYSA-N); CHEMBL4101133 (AHRFPSKABRPTCE-UHFFFAOYSA-N); CHEMBL410222 (CXTGGRMXBGAYHF-FQEVSTJZSA-N); CHEMBL4102678 (FXJXSSSNEZYQFB-XNMGKTSESA-N); CHEMBL4103127 (RJMPNBLDKMGHPA-UHFFFAOYSA-N); CHEMBL4103520 (RHUBXYGWGVODBD-UHFFFAOYSA-N); CHEMBL4170692 (HIRWEADVTAWACU-UHFFFAOYSA-N); CHEMBL4210068 (MTYNXDRAJLIBDC-SLUIUKIVSA-N); CHEMBL4213310 (ZJKIOFVUKLNKSP-WPLLWEEISA-N); CHEMBL4225025 (TWQXMRJSCLJOPP-UHFFFAOYSA-N); CHEMBL4276891 (QXBJHLKMSCVVRS-SEBFSPHVSA-N); CHEMBL4277322 (SOZJKLZVHUFJMU-SEBFSPHVSA-N); CHEMBL4284479 (ZREFSAMKZCTNFF-GOAPWOOUSA-N); CHEMBL4287532 (TWFBNIXTCPAYBH-SEBFSPHVSA-N); CHEMBL4295176 (NBZKHJBPIXOMAL-SEBFSPHVSA-N); CHEMBL4450400 (AYABONFBJMJOPT-UHFFFAOYSA-N); CHEMBL4463657 (UABOZOMXYKTCLE-UHFFFAOYSA-N); CHEMBL4470943 (ATVDZMPSVWPAQR-UHFFFAOYSA-N); CHEMBL4476574 (OLJQKFKCUGKGIW-UHFFFAOYSA-N); CHEMBL44919 (LZWQNHLDOZAEHC-UHFFFAOYSA-N); CHEMBL4515731 (MNXCNJAMZLPOFR-UHFFFAOYSA-N); CHEMBL4529472 (JZFJTJAQQYXTQB-UHFFFAOYSA-N); CHEMBL4540226 (PCQPAKMJSYGXRE-UHFFFAOYSA-N); CHEMBL4543206 (BJEDNWJTPINJID-UHFFFAOYSA-N); CHEMBL4550982 (PDOCIQUPMPPJRM-UHFFFAOYSA-N); CHEMBL4555219 (CSPZQNIRJZLJQO-UHFFFAOYSA-N); CHEMBL4558932 (RKCSJJATGAWCBK-UHFFFAOYSA-N); CHEMBL4570069 (ISACNBGFGVEWCJ-UHFFFAOYSA-N); CHEMBL4582669 (ZCFQSDCBUSJKCY-WEVVVXLNSA-N); CHEMBL4597954 (VGRNFHJXEIFRTK-OUKQBFOZSA-N); CHEMBL473086 (YJNRXXHBRODLRR-UHFFFAOYSA-N); CHEMBL4741117 (JSROZNRJNCKOTF-INIZCTEOSA-N); CHEMBL4781237 (GQJSXAYEZJMZJS-AWEZNQCLSA-N); CHEMBL4783328 (FKUSUQUCJVZNII-UHFFFAOYSA-N); CHEMBL4784249 (WHWUFZRPFZJRIY-UHFFFAOYSA-N); CHEMBL4784821 (GJGSEWVIKCCBLN-AWEZNQCLSA-N); CHEMBL4789947 (NDQOBOLMTKPDHT-BGABXYSRSA-N); CHEMBL4794550 (ISYQHZMNRMKGOH-VJUOEERUSA-N); CHEMBL4800264 (DZBBSCITDWHHOR-UHFFFAOYSA-N); CHEMBL4859716 (WUEJFDOJIBRVBJ-UHFFFAOYSA-N); CHEMBL4860369 (LPJAKJUVEMSSSX-UHFFFAOYSA-N); CHEMBL487137 (HJYWTNKSSXJHQY-UHFFFAOYSA-N); CHEMBL5091267 (NYEZJKVBGQHMLE-UHFFFAOYSA-N); CHEMBL591183 (IENYQAIEIIEHTE-UHFFFAOYSA-N); CHEMBL595894 (OUWGQIQTCUKTEV-ORBVJSQLSA-N); CHEMBL612113 (QUBKKKSIFUOQPQ-UHFFFAOYSA-N); CHEMBL75474 (PAFKTGFSEFKSQG-BLYLLKCRSA-N); CHIR-124 (MOVBBVMDHIRCTG-LJQANCHMSA-N); CID 122412735 (UGDKPWVVBKHRDK-KPWCQOOUSA-N); CID 127020967 (OWPPSPZDYGHPAN-GCNJZUOMSA-N); CID 149494442 (ZFSRXAHDJSCEDS-HXUWFJFHSA-N); CID 372978 (PKVRCIRHQMSYJX-XHKCZRNKSA-N); CID 382054 (MRJQTLJSMQOFTP-UHFFFAOYSA-N); CID 50900822 (BUGBBPQKQPQAAO-UHFFFAOYSA-N); CID 6711227 (KYHUYMLIVQFXRI-KJNZDXKESA-N); CID 6711246 (AWYLYACMGLGJJG-XSSYFPCESA-N); CID 6711248 (AGIMKWVJNZNTTM-WQBKIXSZSA-N); CID 6711321 (HKSZLNNOFSGOKW-VYEKLMEQSA-N); CID 90331177 (ILUKRINUNLAVMH-UHFFFAOYSA-N); CID 9796294 (SXYIRMFQILZOAM-WJMRQGARSA-N); CID 9832825 (AQTQHPDCURKLKT-FCIBFBFCSA-N); CM03 (WHLXIQNGOLIDJQ-UHFFFAOYSA-N); CP-673451 (DEEOXSOLTLIWMG-UHFFFAOYSA-N); CP-724714 (LLVZBTWPGQVVLW-SNAWJCMRSA-N); CP-91149 (HINJNZFCMLSBCI-PKOBYXMFSA-N); CPI-0610 anhydrous (GCWIQUVXWZWCLE-INIZCTEOSA-N); CPI-1205 (HPODOLXTMDHLLC-QGZVFWFLSA-N); CPI-203 (QECMENZMDBOLDR-AWEZNQCLSA-N); CRS-3123 (NNTYBKTXMKBRFA-CQSZACIVSA-N); CTS-1027 (ROSNVSQTEGHUKU-UHFFFAOYSA-N); CUDC-427 (HSHPBORBOJIXSQ-HARLFGEKSA-N); CX-5461 (XGPBJCHFROADCK-UHFFFAOYSA-N); CX-6258 HCl (YYIMMVXTWBIEAG-YHLMHSEJSA-N); Cabazitaxel (BMQGVNUXMIRLCK-OAGWZNDDSA-N); Cabozantinib (ONIQOQHATWINJY-UHFFFAOYSA-N); Cabozantinib S-malate (HFCFMRYTXDINDK-WNQIDUERSA-N); Caffeic acid (QAIPRVGONGVQAS-DUXPYHPUSA-N); Cain's quinolinium ion (PVFUAZCQIHGOAV-UHFFFAOYSA-O) salts; Calanolide (NIDRYBLTWYFCFV-UHFFFAOYSA-N); Calcimycin (HIYAVKIYRIFSCZ-CYEMHPAKSA-N); Calcipotriol (LWQQLNNNIPYSNX-UROSTWAQSA-N); Calcitriol (GMRQFYUYWCNGIN-NKMMMXOESA-N); Callystatin a (QPJTWGLLJWBDQW-KMMMXHTBSA-N); Calpeptin (PGGUOGKHUUUWAF-ROUUACIJSA-N); Camicinal (RZKDEGZIFSJVNA-IBGZPJMESA-N); Camizestrant (WDHOIABIERMLGY-CMJOXMDJSA-N); Camostat (XASIMHXSUQUHLV-UHFFFAOYSA-N); Camptothecin (VSJKWCGYPAHWDS-FQEVSTJZSA-N); Camptothecin hemisuccinate sodium salt ion (YRCRMPLVFWPTIU-JIDHJSLPSA-N) salts; Camptothecin lysinate hel (PHEXRCGDJLXLMB-UKBNZDFYSA-N); Camptothecin phosphate (PRAQDADSDVOMGA-FQEVSTJZSA-N); Camptothecin propylglycinate ester hydrochloride (VYUPLCFEEDAYEN-UQIIZPHYSA-N); Camptothecin, 10-amino-(MVUUMBZAHAKPKQ-UHFFFAOYSA-N); Camptothecin, N-methylglycinate ester (BIBVWHFPHIJOTQ-UHFFFAOYSA-N); Camptothecin,N-diethyl) glycinate (HGVXFMWXNAOLGL-SNYZSRNZSA-N); Camptothecin-20-O—(N,N-dimethyl)glycinate HCl (AHCBDZJRJCTEHB-JIDHJSLPSA-N); Camptothecine sodium ion (HPSUBMDJBRNXKK-VEIFNGETSA-N) salts; Camylofin (RYOOHIUJEJZCFT-UHFFFAOYSA-N); Canagliflozin (XTNGUQKDFGDXSJ-ZXGKGEBGSA-N); Candesartan (HTQMVQVXFRQIKW-UHFFFAOYSA-N); Candesartan cilexetil (GHOSNRCGJFBJIB-UHFFFAOYSA-N); Canertinib (OMZCMEYTWSXEPZ-UHFFFAOYSA-N); Canertinib dihydrochloride (JZZFDCXSFTVOJY-UHFFFAOYSA-N); Cannabidiol (QHMBSVQNZZTUGM-ZWKOTPCHSA-N); Cannabinol (VBGLYOIFKLUMQG-UHFFFAOYSA-N); Canrenone (UJVLDDZCTMKXJK-WNHSNXHDSA-N); Cantharidin (DHZBEENLJMYSHQ-XCVPVQRUSA-N); Capecitabine (GAGWJHPBXLXJQN-UORFTKCHSA-N); Capivasertib (JDUBGYFRJFOXQC-KRWDZBQOSA-N); Capsaicin (YKPUWZUDDOIDPM-SOFGYWHQSA-N); Carbadox (OVGGLBAWFMIPPY-WUXMJOGZSA-N); Carbamazepine (FFGPTBGBLSHEPO-UHFFFAOYSA-N); Carbaryl (CVXBEEMKQHEXEN-UHFFFAOYSA-N); Carbendazim (TWFZGCMQGLPBSX-UHFFFAOYSA-N); Carbetapentane (CFJMRBQWBDQYMK-UHFFFAOYSA-N); Carbimazole (CFOYWRHIYXMDOT-UHFFFAOYSA-N); Carbinoxamine maleate (GVNWHCVWDRNXAZ-BTJKTKAUSA-N); Carbocysteine (GBFLZEXEOZUWRN-VKHMYHEASA-N); Carboplatin (VSRXQHXAPYXROS-UHFFFAOYSA-N); Carboquone (SHHKQEUPHAENFK-UHFFFAOYSA-N); Carboxyamidotriazole (WNRZHQBJSXRYJK-UHFFFAOYSA-N); Carboxyphthalato platinum (IDNIKXCEUZXOCR-UHFFFAOYSA-N); Carfilzomib (BLMPQMFVWMYDKT-NZTKNTHTSA-N); Carmofur (AOCCBINRVIKJHY-UHFFFAOYSA-N); Carmustine (DLGOEMSEDOSKAD-UHFFFAOYSA-N); Carphenazine dimaleate (TVPJGGZLZLUPOB-SPIKMXEPSA-N); Carzenide (UCAGLBKTLXCODC-UHFFFAOYSA-N); Cc-115 (GMYLVKUGJMYTFB-UHFFFAOYSA-N); Cc-223 (UFKLYTOEMRFKAD-UHFFFAOYSA-N); Cdk1/2 Inhibitor III (ARIOBGGRZJITQX-UHFFFAOYSA-N); Cebranopadol (CSMVOZKEWSOFER-UHFFFAOYSA-N); Cediranib (XXJWYDDUDKYVKI-UHFFFAOYSA-N); Cefazolin (MLYYVTUWGNIJIB-BXKDBHETSA-N); Cefetamet pivoxil hcl (XAAOHMIKXULDKJ-LZRHLYMTSA-N); Cefmenoxime hydrochloride (MPTNDTIREFCQLK-UNVJPQNDSA-N); Cefoperazone (GCFBRXLSHGKWDP-XCGNWRKASA-N); Cefotiam hydrochloride (SVSFIELZISOJDT-XRZFDKQNSA-N); Cefpirome sulfate (RKTNPKZEPLCLSF-QHBKFCFHSA-N); Celastrol (KQJSQWZMSAGSHN-JJWQIEBTSA-N); Celecoxib (RZEKVGVHFLEQIL-UHFFFAOYSA-N); Cep-37440 (BCSHRERPHLTPEE-NRFANRHFSA-N); Cephaeline hydrochloride (NWYUGUJIBCMDEB-QJAXAJGISA-N); Cephalothin sodium (VUFGUVLLDPOSBC-XRZFDKQNSA-M); Cepharanthine (YVPXVXANRNDGTA-WDYNHAJCSA-N); Ceralasertib (OHUHVTCQTUDPIJ-JYCIKRDWSA-N); Cerdulatinib (BGLPECHZZQDNCD-UHFFFAOYSA-N); Ceritinib (VERWOWGGCGHDQE-UHFFFAOYSA-N); Cerivastatin sodium (GPUADMRJQVPIAS-QCVDVZFFSA-M); Cerubidin (STQGQHZAVUOBTE-UHFFFAOYSA-N); Cetalkonium bromide (DLNWMWYCSOQYSQ-UHFFFAOYSA-M); Cetalkonium chloride (SXPWTBGAZSPLHA-UHFFFAOYSA-M); Cetirizine hydrochloride (PGLIUCLTXOYQMV-UHFFFAOYSA-N); Cetrimide (CXRFDZFCGOPDTD-UHFFFAOYSA-M); Cetrimonium bromide (LZZYPRNAOMGNLH-UHFFFAOYSA-M); Cetylpyridinium bromide (DVBJBNKEBPCGSY-UHFFFAOYSA-M); Cetylpyridinium chloride (YMKDRGPMQRFJGP-UHFFFAOYSA-M); Cetylpyridinium chloride monohydrate (NFCRBQADEGXVDL-UHFFFAOYSA-M); Cetylpyridinium ion (NEUSVAOJNUQRTM-UHFFFAOYSA-N) salts; Cevipabulin (ZUZPCOQWSYNWLU-VIFPVBQESA-N); Chaetochromin (RHNVLFNWDGWACV-UHFFFAOYSA-N); Chaetocin (PZPPOCZWRGNKIR-UHFFFAOYSA-N); Chelerythrine chloride (WEEFNMFMNMASJY-UHFFFAOYSA-M); Chidamide (WXHHICFWKXDFOW-BJMVGYQFSA-N); Chlophedianol (WRCHFMBCVFFYEQ-UHFFFAOYSA-N); Chlorambucil (JCKYGMPEJWAADB-UHFFFAOYSA-N); Chloramphenicol (WIIZWVCIJKGZOK-RKDXNWHRSA-N); Chlorcyclizine hydrochloride (MSIJLVMSKDXAQN-UHFFFAOYSA-N); Chlordiazepoxide (BUCORZSTKDOEKQ-UHFFFAOYSA-N); Chlorhexidine hcl (WJLVQTJZDCGNJN-UHFFFAOYSA-N); Chlormadinone acetate (QMBJSIBWORFWQT-DFXBJWIESA-N); Chloro-9-[[3-(diethylamino)propyl]amino]-2-methoxyacridine, dihydrate (JQXKUFYMRDWBEU-UHFFFAOYSA-N); Chloro-IB-MECA (IPSYPUKKXMNCNQ-PFHKOEEOSA-N); Chlorolissoclimide (LCBZIVZSFYGPBC-LWHFJPTFSA-N); Chloroquine (WHTVZRBIWZFKQO-UHFFFAOYSA-N); Chloroquine phosphate (QKICWELGRMTQCR-UHFFFAOYSA-N); Chlorotrianisene (BFPSDSIWYFKGBC-UHFFFAOYSA-N); Chloroxine (WDFKMLRRRCGAKS-UHFFFAOYSA-N); Chlorozorubicin hydrochloride (TWPKQOAFJZUCMV-FYBMHLGZSA-N); Chlorphenoxamine (KKHPNPMTPORSQE-UHFFFAOYSA-N); Chlorpromazine (ZPEIMTDSQAKGNT-UHFFFAOYSA-N); Chlorpromazine hydrochloride (FBSMERQALIEGJT-UHFFFAOYSA-N); Chlorpropamide (RKWGIWYCVPQPMF-UHFFFAOYSA-N); Chlorprothixen (WSPOMRSOLSGNFJ-VGOFMYFVSA-N); Chlorprothixene hydrochloride (YWKRLOSRDGPEJR-KIUKIJHYSA-N); Chlorquinaldol (GPTXWRGISTZRIO-UHFFFAOYSA-N); Chlorsulfaquinoxaline (CTSNHMQGVWXIEG-UHFFFAOYSA-N); Chlortetracycline hydrochloride (QYAPHLRPFNSDNH-MRFRVZCGSA-N); Cholecalciferol (QYSXJUFSXHHAJI-YRZJJWOYSA-N); Chromomycin (ZYVSOIYQKUDENJ-MNGCYDFMSA-N); Cianidanol (PFTAWBLQPZVEMU-DZGCQCFKSA-N); Ciclesonide (LUKZNWIVRBCLON-GXOBDPJESA-N); Ciclobendazole (OXLKOMYHDYVIDM-UHFFFAOYSA-N); Ciclopirox (SCKYRAXSEDYPSA-UHFFFAOYSA-N); Ciclopirox olamine (MBRHNTMUYWQHMR-UHFFFAOYSA-N); Ciclosporin (PMATZTZNYRCHOR-CGLBZJNRSA-N); Cilengitide (AMLYAMJWYAIXIA-VWNVYAMZSA-N); Cilnidipine (KJEBULYHNRNJTE-DHZHZOJOSA-N); Cilomilast (CFBUZOUXXHZCFB-UHFFFAOYSA-N); Cilostazol (RRGUKTPIGVIEKM-UHFFFAOYSA-N); Cinacalcet (VDHAWDNDOKGFTD-MRXNPFEDSA-N); Cinacalcet hydrochloride (QANQWUQOEJZMLL-PKLMIRHRSA-N); Cinchonine (KMPWYEUPVWOPIM-QAMTZSDWSA-N); Cinchophen (YTRMTPPVNRALON-UHFFFAOYSA-N); Cinerubin a hcl (ACPRCYNYDVWPJV-BLZBDMHNSA-N); Cinerubin b hcl (ZBDDFHXUDIPRSM-UHFFFAOYSA-N); Cinerubine a (STUJMJDONFVTGM-CEPMKHARSA-N); Cinnarizine (DERZBLKQOCDDDZ-JLHYYAGUSA-N); Ciprofibrate (KPSRODZRAIWAKH-UHFFFAOYSA-N); Cis-4-{[2-(1 h-Benzimidazol-5-Ylamino)quinazolin-8-Yl]oxy}cyclohexanol (FYWRWBSYRGSWIQ-UHFFFAOYSA-N); Cisapride (DCSUBABJRXZOMT-UHFFFAOYSA-N); Cisapride monohydrate [orange book](QBYYXIDJOFZORM-LBPAWUGGSA-N); Citalopram (WSEQXVZVJXJVFP-UHFFFAOYSA-N); Citarinostat (VLIUIBXPEDFJRF-UHFFFAOYSA-N); Citric acid (KRKNYBCHXYNGOX-UHFFFAOYSA-N); Cladribine (PTOAARAWEBMLNO-KVQBGUIXSA-N); Clemastine (YNNUSGIPVFPVBX-NHCUHLMSSA-N); Clemastine fumarate (PMGQWSIVQFOFOQ-YKVZVUFRSA-N); Climbazole (OWEGWHBOCFMBLP-UHFFFAOYSA-N); Clindamycin palmitate hydrochloride (GTNDZRUWKHDICY-DJHAJVGHSA-N); Clioquinol (QCDFBFJGMNKBDO-UHFFFAOYSA-N); Clobetasol (FCSHDIVRCWTZOX-DVTGEIKXSA-N); Clobetasol propionate (CBGUOGMQLZIXBE-XGQKBEPLSA-N); Clobetasone butyrate (FBRAWBYQGRLCEK-AVVSTMBFSA-N); Clofarabine (WDDPHFBMKLOVOX-AYQXTPAHSA-N); Clofazimine (WDQPAMHFFCXSNU-UHFFFAOYSA-N); Clofenotane (YVGGHNCTFXOJCH-UHFFFAOYSA-N); Clofibrate (KNHUKKLJHYUCFP-UHFFFAOYSA-N); Clofoctol (HQVZOORKDNCGCK-UHFFFAOYSA-N); Clomesone (SEHSPJCWCBQHPF-UHFFFAOYSA-N); Clometacin (DGMZLCLHHVYDIS-UHFFFAOYSA-N); Clomifene (GKIRPKYJQBWNGO-UHFFFAOYSA-N); Clomiphene (GKIRPKYJQBWNGO-OCEACIFDSA-N); Clomiphene citrate (PYTMYKVIJXPNBD-OQKDUQJOSA-N); Clomipramine (GDLIGKIOYRNHDA-UHFFFAOYSA-N); Clomipramine hydrochloride (WIMWMKZEIBHDTH-UHFFFAOYSA-N); Cloperastine hydrochloride (UNPLRYRWJLTVAE-UHFFFAOYSA-N); Clopidogrel (GKTWGGQPFAXNFI-HNNXBMFYSA-N); Closantel (JMPFSEBWVLAJKM-UHFFFAOYSA-N); Clothiapine (KAAZGXDPUNNEFN-UHFFFAOYSA-N); Clotrimazole (VNFPBHJOKIVQEB-UHFFFAOYSA-N); Clozapine (QZUDBNBUXVUHMW-UHFFFAOYSA-N); Cndac (DCYBPMFXJCWXNB-JWIUVKOKSA-N); Cobimetinib (BSMCAPRUBJMWDF-KRWDZBQOSA-N); Cochliobolin a (MWYYLZRWWNBROW-FMQZQXMHSA-N); Codeine hydrochloride (NUXLENPAZQNFAM-FFHNEAJVSA-N); Coenzyme Q10 (ACTIUHUUMQJHFO-UPTCCGCDSA-N); Colchiceinamid (JWDFWIZNGSOUGC-INIZCTEOSA-N); Colchiceine (PRGILOMAMBLWNG-HNNXBMFYSA-N); Colchicine (IAKHMKGGTNLKSZ-INIZCTEOSA-N); Colchicine, 17-chloro-(XBFWCWRZDWNRIE-HNNXBMFYSA-N); Colchinol, N-acetyl-(WJJZQSCOTJYYSP-INIZCTEOSA-N); Colestipol (GMRWGQCZJGVHKL-UHFFFAOYSA-N); Combretastatin A-1 (YUSYSJSHVJULID-WAYWQWQTSA-N); Combretastatin A4 (HVXBOLULGPECHP-WAYWQWQTSA-N); Conjuncain (PRGUDWLMFLCODA-UHFFFAOYSA-N); Copanlisib (MWYDSXOGIBMAET-UHFFFAOYSA-N); Coralyne chloride (PDYBUYVOPAJLKP-UHFFFAOYSA-M); Cordycepin (OFEZSBMBBKLLBJ-BAJZRUMYSA-N); Cordyheptapeptide a (ZXPWAGZHBVVLJM-PPLXTMPISA-N); Cortexolone 17alpha-propionate (GPNHMOZDMYNCPO-PDUMRIMRSA-N); Corticosterone (OMFXVFTZEKFJBZ-HJTSIMOOSA-N); Cortisone acetate (ITRJWOMZKQRYTA-RFZYENFJSA-N); Cortivazol (RKHQGWMMUURILY-UHRZLXHJSA-N); Coumaphos (BXNANOICGRISHX-UHFFFAOYSA-N); Coumarin (ZYGHJZDHTFUPRJ-UHFFFAOYSA-N); Creatinine (DDRJAANPRJIHGJ-UHFFFAOYSA-N); Crenolanib (DYNHJHQFHQTFTP-UHFFFAOYSA-N); Cridanimod (UOMKBIIXHQIERR-UHFFFAOYSA-N); Crinecerfont (IEAKXXNRGSLYTQ-DEOSSOPVSA-N); Crisaborole (USZAGAREISWJDP-UHFFFAOYSA-N); Crizotinib (KTEIFNKAUNYNJU-GFCCVEGCSA-N); Cryptopleurine (RSHYSOGXGSUUIJ-OAHLLOKOSA-N); Cucurbitacin a (IHTCCHVMPGDDSL-IVNGUWCNSA-N); Cucurbitacin d (SRPHMISUTWFFKJ-QJNWWGCFSA-N); Cucurbitacin e (NDYMQXYDSVBNLL-MUYMLXPFSA-N); Cucurbitacin i (NISPVUDLMHQFRQ-MKIKIEMVSA-N); Cudc-101 (PLIVFNIUGLLCEK-UHFFFAOYSA-N); Curacozole (GARMIUZCKGLXIH-UHFFFAOYSA-N); Curcumin (VFLDPWHFBUODDF-FCXRPNKRSA-N); Cvt-313 (NQVIIUBWMBHLOZ-UHFFFAOYSA-N); Cvt-6883 (KOYXXLLNCXWUNF-UHFFFAOYSA-N); Cyanocycline a (KDWXMIJUXUSUMZ-UHFFFAOYSA-N); Cyanomorpholinoadriamycin (YIMDLWDNDGKDTJ-UHFFFAOYSA-N); Cyanomorpholinoadriamycin (YIMDLWDNDGKDTJ-WJRCOUIISA-N); Cyclo-C(LOALESLYJVZMHL-UHFFFAOYSA-N); Cyclobenzaprine (JURKNVYFZMSNLP-UHFFFAOYSA-N); Cyclobenzaprine hydrochloride (VXEAYBOGHINOKW-UHFFFAOYSA-N); Cyclodisone (RYIRMSRYCSMGJA-UHFFFAOYSA-N); Cycloguanil hydrochloride (MOUAPRKJJUXEIE-UHFFFAOYSA-N); Cycloheximide (YPHMISFOHDHNIV-FSZOTQKASA-N); Cycloleucine (NILQLFBWTXNUOE-UHFFFAOYSA-N); Cyclopentenyl cytosine (DUJGMZAICVPCBJ-VDAHYXPESA-N); Cyclophosphamide (CMSMOCZEIVJLDB-UHFFFAOYSA-N); Cyclophosphamide monohydrate (PWOQRKCAHTVFLB-UHFFFAOYSA-N); Cydectin (YZBLFMPOMVTDJY-CBYMMZEQSA-N); Cylocide (KCURWTAZOZXKSJ-UHFFFAOYSA-N); Cynarine (YDDUMTOHNYZQPO-RVXRWRFUSA-N); Cyproheptadine (JJCFRYNCJDLXIK-UHFFFAOYSA-N); Cyproheptadine hydrochloride (ZPMVNZLARAEGHB-UHFFFAOYSA-N); Cyproterone acetate (UWFYSQMTEOIJJG-FDTZYFLXSA-N); Cytarabine (UHDGCWIWMRVCDJ-CCXZUQQUSA-N); Cytarabine hydrochloride (KCURWTAZOZXKSJ-JBMRGDGGSA-N); Cytidine (UHDGCWIWMRVCDJ-XVFCMESISA-N); Cytidine, 2′-deoxy-2′,2′-difluoro-N-(1-oxo-2-propylpentyl)-(MEOYFIHNRBNEPI-UXIGCNINSA-N); Cytidine,3′-O-(dibutylstannylene)-(USALKYCONOYHAU-UHFFFAOYSA-N); Cytisine (ANJTVLIZGCUXLD-DTWKUNHWSA-N); Cytochalasin e (LAJXCUNOQSHRJO-ZYGJITOWSA-N); Cytosine arabinoside monophosphate (IERHLVCPSMICTF-UHFFFAOYSA-N); DDUG (FPIKMLZWCUBRIP-NJFKTRIISA-N); DHAD (MKCBCZDNNZPMCG-UHFFFAOYSA-N); DL-Amethopterin (FBOZXECLQNJBKD-UHFFFAOYSA-N); DL-Arabinose (PYMYPHUHKUWMLA-UHFFFAOYSA-N); DL-N(Me2)Val-DL-Val-DL-N(Me)Val-DL-Pro-DL-Pro-DL-OVal-Unk (LQKSHSFQQRCAFW-UHFFFAOYSA-N); DTIC-Dome (FDKXTQMXEQVLRF-ZHACJKMWSA-N); DTXSID60956751 (HKAMJBJQZVUJFZ-UHFFFAOYSA-N); DX-52-1 (YGWHMSNGVVTUIT-UHFFFAOYSA-N); Dabigatran (YBSJFWOBGCMAKL-UHFFFAOYSA-N); Dabrafenib (BFSMGDJOXZAERB-UHFFFAOYSA-N); Dacarbazine (FDKXTQMXEQVLRF-UHFFFAOYSA-N); Daclatasvir (FKRSSPOQAMALKA-CUPIEXAXSA-N); Dacomitinib (LVXJQMNHJWSHET-AATRIKPKSA-N); Dactinomycin (RJURFGZVJUQBHK-IIXSONLDSA-N); Dactinomycin d (RJURFGZVJUQBHK-GIUPLMLQSA-N); Dactolisib (JOGKUKXHTYWRGZ-UHFFFAOYSA-N); Daidzein (ZQSIJRDFPHDXIC-UHFFFAOYSA-N); Dalcetrapib (YZQLWPMZQVHJED-UHFFFAOYSA-N); Danazol (POZRVZJJTULAOH-LHZXLZLDSA-N); Daniquidone (SRIOCKJKFXAKHK-UHFFFAOYSA-N); Danthron (QBPFLULOKWLNNW-UHFFFAOYSA-N); Dantrolene sodium (KSRLIXGNPXAZHD-HAZZGOGXSA-M); Danusertib (XKFTZKGMDDZMJI-HSZRJFAPSA-N); Daphnetin (ATEFPOUAMCWAQS-UHFFFAOYSA-N); Dapivirine (ILAYIAGXTHKHNT-UHFFFAOYSA-N); Daporinad (KPBNHDGDUADAGP-VAWYXSNFSA-N); Dapoxetine (USRHYDPUVLEVMC-FQEVSTJZSA-N); Daprodustat (RUEYEZADQJCKGV-UHFFFAOYSA-N); Dapsone (MQJKPEGWNLWLTK-UHFFFAOYSA-N); Dapt (DWJXYEABWRJFSP-XOBRGWDASA-N); Darapladib (WDPFJWLDPVQCAJ-UHFFFAOYSA-N); Darifenacin hydrobromide (UQAVIASOPREUIT-VQIWEWKSSA-N); Darolutamide (BLIJXOOIHRSQRB-PXYINDEMSA-N); Darovasertib (XXJXHXJWQSCNPX-UHFFFAOYSA-N); Darunavir (CJBJHOAVZSMMDJ-HEXNFIEUSA-N); Dasatinib (ZBNZXTGUTAYRHI-UHFFFAOYSA-N); Daunoblastin (GUGHGUXZJWAIAS-CFCXLQPVSA-N); Daunorubicin (STQGQHZAVUOBTE-VGBVRHCVSA-N); Daunorubicin hydrochloride (GUGHGUXZJWAIAS-QQYBVWGSSA-N); Daunorubicin hydrochloride (GUGHGUXZJWAIAS-UHFFFAOYSA-N); Debio 0932 (RVJIQAYFTOPTKK-UHFFFAOYSA-N); Debio-1347 (BEMNJULZEQTDJY-UHFFFAOYSA-N); Decamine ion (IHLKQCODTQXANL-UHFFFAOYSA-O) salts; Decernotinib (ASUGUQWIHMTFJL-QGZVFWFLSA-N); Decitabine (XAUDJQYHKZQPEU-KVQBGUIXSA-N); Defactinib (FWLMVFUGMHIOAA-UHFFFAOYSA-N); Deferasirox (BOFQWVMAQOTZIW-UHFFFAOYSA-N); Deferoxamine (UBQYURCVBFRUQT-UHFFFAOYSA-N); Deferoxamine mesylate (IDDIJAWJANBQLJ-UHFFFAOYSA-N); Deflazacort (FBHSPRKOSMHSIF-GRMWVWQJSA-N); Dehydroacetic acid (PGRHXDWITVMQBC-UHFFFAOYSA-N); Dehydroepiandrosterone (FMGSKLZLMKYGDP-USOAJAOKSA-N); Dehydroepiandrosterone acetate (NCMZQTLCXHGLOK-ZKHIMWLXSA-N); Dehydrospongistatin 2 (CBSUYSJPVZOMOZ-LTEJQQKOSA-N); Delanzomib (SJFBTAPEPRWNKH-CCKFTAQKSA-N); Deoxybouvardin (VXVGFMUNENQGFW-UHFFFAOYSA-N); Deoxycholic acid (KXGVEGMKQFWNSR-LLQZFEROSA-N); Deoxycorticosterone acetate (VPGRYOFKCNULNK-ACXQXYJUSA-N); Deoxydoxorubicin (RCFNNLSZHVHCEK-IMHLAKCZSA-N); Deoxypodophyllotoxin (ZGLXUQQMLLIKAN-SVIJTADQSA-N); Dequalinium chloride (LTNZEXKYNRNOGT-UHFFFAOYSA-N); Dequalinium ion (PCSWXVJAIHCTMO-UHFFFAOYSA-P) salts; Derazantinib (KPJDVVCDVBFRMU-AREMUKBSSA-N); Desacetylcolchicine d-tartrate (JLRWNBXMQCGSEJ-APBURCQWSA-N); Desacetylvinblastine amide (HHJUWIANJFBDHT-ZVTSDNJWSA-N); Deserpidine (CVBMAZKKCSYWQR-WCGOZPBSSA-N); Desglucocheirotoxin (HULMNSIAKWANQO-UHFFFAOYSA-N); Desipramine hydrochloride (XAEWZDYWZHIUCT-UHFFFAOYSA-N); Deslanoside (OBATZBGFDSVCJD-LALPQLPRSA-N); Desloratadine (JAUOIFJMECXRGI-UHFFFAOYSA-N); Desmethoxy-B-peltatin-A methyl ether (XPWTYDWZZDAXDF-UHFFFAOYSA-N); Desogestrel (RPLCPCMSCLEKRS-BPIQYHPVSA-N); Detomidine hydrochloride (OIWRDXKNDCJZSM-UHFFFAOYSA-N); Dexamethasone (UREBDLICKHMUKA-CXSFZGCWSA-N); Dexamethasone acetate (AKUJBENLRBOFTD-RPRRAYFGSA-N); Dexbrompheniramine maleate (SRGKFVAASLQVBO-DASCVMRKSA-N); Dexnorgestrel acetime (KIQQMECNKUGGKA-NMYWJIRASA-N); Dexrazoxane (BMKDZUISNHGIBY-ZETCQYMHSA-N); Dextromethorphan hydrobromide monohydrate (STTADZBLEUMJRG-IKNOHUQMSA-N); Dhaq diacetate salt (MXBYMBHGTLEGPX-UHFFFAOYSA-N); Diacerein (TYNLGDBUJLVSMA-UHFFFAOYSA-N); Diacetamate (UJAOSPFULOFZRR-UHFFFAOYSA-N); Diacetoxyscirpenol (AUGQEEXBDZWUJY-UHFFFAOYSA-N); Diamthazole (WGMYEOIMVYADRJ-UHFFFAOYSA-N); Dianhydrodulcitol (AAFJXZWCNVJTMK-UHFFFAOYSA-N); Diaveridine (LDBTVAXGKYIFHO-UHFFFAOYSA-N); Diazepam (AAOVKJBEBIDNHE-UHFFFAOYSA-N); Diaziquone (WVYXNIXAMZOZFK-UHFFFAOYSA-N); Diazoacetate(ester) L-serine ion (LMTILILZEHPNAP-UHFFFAOYSA-O) salts; Diazonamide a (YKBUODYYSZSEIY-PLSHLZFXSA-N); Dibenzyline (VBCPVIWPDJVHAN-UHFFFAOYSA-N); Dibromsalan (CTFFKFYWSOSIAA-UHFFFAOYSA-N); Dibucaine (PUFQVTATUTYEAL-UHFFFAOYSA-N); Dichlorophen (MDNWOSOZYLHTCG-UHFFFAOYSA-N); Dichlorphenamide (GJQPMPFPNINLKP-UHFFFAOYSA-N); Dichlorvos (OEBRKCOSUFCWJD-UHFFFAOYSA-N); Diclazuril (ZSZFUDFOPOMEET-UHFFFAOYSA-N); Dicumarol (DOBMPNYZJYQDGZ-UHFFFAOYSA-N); Dicyclohexyltin oxide (QULMZVWEGVTWJY-UHFFFAOYSA-N); Dicyclomine (CURUTKGFNZGFSE-UHFFFAOYSA-N); Dicyclomine hydrochloride (GUBNMFJOJGDCEL-UHFFFAOYSA-N); Didanosine (BXZVVICBKDXVGW-NKWVEPMBSA-N); Didemnin A, N-L-prolyl-2-L-allothreonine (MMUFNCTVUTWOJE-LKZKYIBOSA-N); Didesmethylrocaglamide (RMNPQEWLGQURNX-PXIJUOARSA-N); Dienestrol (NFDFQCUYFHCNBW-SCGPFSFSSA-N); Dienochlor (LWLJUMBEZJHXHV-UHFFFAOYSA-N); Diethyl 6-methoxy-5,7-dihydroindolo[2,3-b]carbazole-2,10-dicarboxylate (BMTPVPNVQOYGAP-UHFFFAOYSA-N); Diethylhomospermine (QXOCYGPVDXDFLC-UHFFFAOYSA-N); Diethylstilbestrol (RGLYKWWBQGJZGM-ISLYRVAYSA-N); Diflomotecan (LFQCJSBXBZRMTN-OAQYLSRUSA-N); Diflucortolone valerate (HHJIUUAMYGBVSD-YTFFSALGSA-N); Diflunisal (HUPFGZXOMWLGNK-UHFFFAOYSA-N); Digitoxin (WDJUZGPOPHTGOT-XUDUSOBPSA-N); Digoxigenin (SHIBSTMRCDJXLN-KCZCNTNESA-N); Digoxin (LTMHDMANZUZIPE-PUGKRICDSA-N); Dihydralazine (VQKLRVZQQYVIJW-UHFFFAOYSA-N); Dihydroartemisinin ethyl ether (NLYNIRQVMRLPIQ-BVCZABNKSA-N); Dihydrobezofuran (HDONJQIYHGHIJV-ZZXKWVIFSA-N); Dihydrocapsaicin (XJQPQKLURWNAAH-UHFFFAOYSA-N); Dihydroergocristine (DEQITUUQPICUMR-HJPBWRTMSA-N); Dihydroergocristine mesylate (SPXACGZWWVIDGR-SPZWACKZSA-N); Dihydroergotamine (LUZRJRNZXALNLM-JGRZULCMSA-N); Dihydromyricetin (KJXSIXMJHKAJOD-LSDHHAIUSA-N); Dihydroqinghaosu (BJDCWCLMFKKGEE-KXTPALSWSA-N); Dihydrotachysterol (ILYCWAKSDCYMBB-OPCMSESCSA-N); Dihydrotriazine derivative (JNVLQWBZTYBYKN-UHFFFAOYSA-N); Diloxanide furoate (BDYYDXJSHYEDGB-UHFFFAOYSA-N); Diltiazem (HSUGRBWQSSZJOP-RTWAWAEBSA-N); Dimemorfan phosphate (ODJHDWLIOUGPPA-URVXVIKDSA-N); Dimercaprol (WQABCVAJNWAXTE-UHFFFAOYSA-N); Dimesna (KQYGMURBTJPBPQ-UHFFFAOYSA-L); Dimethyl fumarate (LDCRTTXIJACKKU-ONEGZZNKSA-N); Dimethyl sulfoxide (IAZDPXIOMUYVGZ-UHFFFAOYSA-N); Dimethyldioctylammonium chloride (FARBQUXLIQOIDY-UHFFFAOYSA-M); Dimetotiazine (VWNWVCJGUMZDIU-UHFFFAOYSA-N); Dinaciclib (PIMQWRZWLQKKBJ-SFHVURJKSA-N); Dioscin (VNONINPVFQTJOC-ZGXDEBHDSA-N); Diosmetin (MBNGWHIJMBWFHU-UHFFFAOYSA-N); Dipentamethylenethiuram disulfide (KNBRWWCHBRQLNY-UHFFFAOYSA-N); Diperodon hydrochloride (OWULVAZDMWJBLB-UHFFFAOYSA-N); Diphenhydramine (ZZVUWRFHKOJYTH-UHFFFAOYSA-N); Diphenhydramine hydrochloride (PCHPORCSPXIHLZ-UHFFFAOYSA-N); Diphenidol (OGAKLTJNUQRZJU-UHFFFAOYSA-N); Diphenyl-bis(8-quinolylsulfanyl)stannane (QSTLOYBHYBPUMN-UHFFFAOYSA-L); Diphenylpropoxyacetic acid 1-methyl-4-piperidyl ester hydrochloride (KFUJMHHNLGCTIJ-UHFFFAOYSA-N); Diphenylpyraline hydrochloride (LPRLDRXGWKXRMQ-UHFFFAOYSA-N); Dipyridamole (IZEKFCXSFNUWAM-UHFFFAOYSA-N); Dipyrithione (ZHDBTKPXEJDTTQ-UHFFFAOYSA-N); Disulfiram (AUZONCFQVSMFAP-UHFFFAOYSA-N); Dithiazanine iodide (MNQDKWZEUULFPX-UHFFFAOYSA-M); Dobutamine (JRWZLRBJNMZMFE-UHFFFAOYSA-N); Dobutamine hydrochloride (BQKADKWNRWCIJL-UHFFFAOYSA-N); Docetaxel (ZDZOTLJHXYCWBA-VCVYQWHSSA-N); Docetaxel (taxotere) (ZDZOTLJHXYCWBA-JXHJHTFNSA-N); Docetaxel trihydrate (XCDIRYDKECHIPE-QHEQPUDQSA-N); Docosahexaenoic acid (MBMBGCFOFBJSGT-KUBAVDMBSA-N); Dofequidar (KLWUUPVJTLHYIM-UHFFFAOYSA-N); Dofetilide (IXTMWRCNAAVVAI-UHFFFAOYSA-N); Dolasetron mesylate (PSGRLCOSIXJUAL-ZPQKBBRKSA-N); Dolastatin 10 (OFDNQWIFNXBECV-VFSYNPLYSA-N); Dolastatin 11 (XOSLDLHSPLPVME-FKLVWXFWSA-N); Dolutegravir (RHWKPHLQXYSBKR-BMIGLBTASA-N); Domatinostat (PRXXYMVLYKJITB-IZZDOVSWSA-N); Domiphen bromide (OJIYIVCMRYCWSE-UHFFFAOYSA-M); Domperidone (FGXWKSZFVQUSTL-UHFFFAOYSA-N); Donepezil hydrochloride (XWAIAVWHZJNZQQ-UHFFFAOYSA-N); Dopamine (VYFYYTLLBUKUHU-UHFFFAOYSA-N); Dopamine hydrochloride (CTENFNNZBMHDDG-UHFFFAOYSA-N); Dopexamine (RYBJORHCUPVNMB-UHFFFAOYSA-N); Doramapimod (MVCOAUNKQVWQHZ-UHFFFAOYSA-N); Doramectin (QLFZZSKTJWDQOS-YDBLARSUSA-N); Dorzolamide (IAVUPMFITXYVAF-XPUUQOCRSA-N); Dovitinib (PIQCTGMSNWUMAF-UHFFFAOYSA-N); Doxazosin (RUZYUOTYCVRMRZ-UHFFFAOYSA-N); Doxazosin mesylate (VJECBOKJABCYMF-UHFFFAOYSA-N); Doxepin hydrochloride (MHNSPTUQQIYJOT-SJDTYFKWSA-N); Doxercalciferol (HKXBNHCUPKIYDM-CGMHZMFXSA-N); Doxercalciferol (hectorol) (HKXBNHCUPKIYDM-ZKVKAWQFSA-N); Doxifluridine (ZWAOHEXOSAUJHY-ZIYNGMLESA-N); Doxo-emch (OBMJQRLIQQTJLR-LBMCFUDOSA-N); Doxorubicin (AOJJSUZBOXZQNB-TZSSRYMLSA-N); Doxorubicin hydrochloride (MWWSFMDVAYGXBV-RUELKSSGSA-N); Doxycycline (SGKRLCUYIXIAHR-AKNGSSGZSA-N); Doxycycline hydrochloride (PTNZGHXUZDHMIQ-CVHRZJFOSA-N); Doxylamine succinate (KBAUFVUYFNWQFM-UHFFFAOYSA-N); Doxypyrromycin hydrochloride (KTSRCTOUNGGQEI-XBLNTETQSA-N); Drofenine hydrochloride (WIELVDXKOYPANK-UHFFFAOYSA-N); Drometrizole (MCPKSFINULVDNX-UHFFFAOYSA-N); Dronabinol (CYQFCXCEBYINGO-IAGOWNOFSA-N); Dronedarone (ZQTNQVWKHCQYLQ-UHFFFAOYSA-N); Droperidol (RMEDXOLNCUSCGS-UHFFFAOYSA-N); Drospirenone (METQSPRSQINEEU-HXCATZOESA-N); Drostanolone propionate (NOTIQUSPUUHHEH-UXOVVSIBSA-N); Dubermatinib (YUAALFPUEOYPNX-UHFFFAOYSA-N); Duloxetine (ZEUITGRIYCTCEM-KRWDZBQOSA-N); Duloxetine hydrochloride (BFFSMCNJSOPUAY-LMOVPXPDSA-N); Duvelisib (SJVQHLPISAIATJ-ZDUSSCGKSA-N); Dyclonine (BZEWSEKUUPWQDQ-UHFFFAOYSA-N); Dyclonine hydrochloride (KNZADIMHVBBPOA-UHFFFAOYSA-N); Dynamax (BJDCWCLMFKKGEE-CMDXXVQNSA-N); E100 (ZIUSSTSXXLLKKK-KOBPDPAPSA-N); ENMD-2076 (BLQYVHBZHAISJM-CMDGGOBGSA-N); EPH-116 (JNNXERNBPXXNLK-WQRHYEAKSA-N); EPZ005687 (ZOIBZSZLMJDVDQ-UHFFFAOYSA-N); EPZ015666 (ZKXZLIFRWWKZRY-KRWDZBQOSA-N); ETC-206 (FWRFPHJSGLYXTD-UHFFFAOYSA-N); Ebastine (MJJALKDDGIKVBE-UHFFFAOYSA-N); Ebselen (DYEFUKCXAQOFHX-UHFFFAOYSA-N); Ebvaciclib (QIEKHLDZKRQLLN-FOIQADDNSA-N); Echinomycin (AUJXLBOHYWTPFV-UHFFFAOYSA-N); Econazole (LEZWWPYKPKIXLL-UHFFFAOYSA-N); Econazole nitrate (DDXORDQKGIZAME-UHFFFAOYSA-N); Ecopipam (DMJWENQHWZZWDF-PKOBYXMFSA-N); Ecteinascidin 729 (UPGCDKVJPIRNTG-HKGVKFBLSA-N); Edaravone (QELUYTUMUWHWMC-UHFFFAOYSA-N); Edotecarin (QMVPQBFHUJZJCS-NTKFZFFISA-N); Efavirenz (XPOQHMRABVBWPR-ZDUSSCGKSA-N); Eflornithine (VLCYCQAOQCDTCN-UHFFFAOYSA-N); Eganelisib (XUMALORDVCFWKV-IBGZPJMESA-N); Elacestrant (SIFNOOUKXBRGGB-AREMUKBSSA-N); Elacridar (OSFCMRGOZNQUSW-UHFFFAOYSA-N); Eldisine (COFJBSXICYYSKG-OAUVCNBTSA-N); Elesclomol (BKJIXTWSNXCKJH-UHFFFAOYSA-N); Eletriptan (PWVXXGRKLHYWKM-LJQANCHMSA-N); Elimusertib (YBXRSCXGRPSTMW-CYBMUJFWSA-N); Eliprodil (GGUSQTSTQSHJAH-UHFFFAOYSA-N); Ellagic acid (AFSDNFLWKVMVRB-UHFFFAOYSA-N); Ellipticine (CTSPAMFJBXKSOY-UHFFFAOYSA-N); Eloxatin (tn) (ZROHGHOFXNOHSO-BNTLRKBRSA-L); Elsamicin (MGQRRMONVLMKJL-UHFFFAOYSA-N); Eltanexor (JFBAVWVBLRIWHM-AWNIVKPZSA-N); Eltrombopag (SVOQIEJWJCQGDQ-UHFFFAOYSA-N); Elvitegravir (JUZYLCPPVHEVSV-LJQANCHMSA-N); Elvucitabine (HSBKFSPNDWWPSL-VDTYLAMSSA-N); Emavusertib (SJHNWSAWWOAWJH-MRXNPFEDSA-N); Embelin (IRSFLDGTOHBADP-UHFFFAOYSA-N); Emedastine difumarate (FWLKKPKZQYVAFR-LVEZLNDCSA-N); Emetine hydrochloride (HUEYSSLYFJVUIS-MRFSYGAJSA-N); Emodin (RHMXXJGYXNZAPX-UHFFFAOYSA-N); Empesertib (NRJKIOCCERLIDG-GOSISDBHSA-N); Emricasan (SCVHJVCATBPIHN-SJCJKPOMSA-N); Enasidenib (DYLUUSLLRIQKOE-UHFFFAOYSA-N); Encainide hydrochloride (OJIIZIWOLTYOBS-UHFFFAOYSA-N); Enclomiphene citrate (PYTMYKVIJXPNBD-BTKVJIOYSA-N); Encorafenib (CMJCXYNUCSMDBY-ZDUSSCGKSA-N); Endoxifen (MHJBZVSGOZTKRH-IZHYLOQSSA-N); Endoxifen hydrochloride (RPFIMPDXTABYCN-BJFQDICYSA-N); Endurobol (YDBLKRPLXZNVNB-UHFFFAOYSA-N); Enobosarm (JNGVJMBLXIUVRD-SFHVURJKSA-N); Enoxacin (IDYZIJYBMGIQMJ-UHFFFAOYSA-N); Enoximone (ZJKNESGOIKRXQY-UHFFFAOYSA-N); Enoxolone (MPDGHEJMBKOTSU-YKLVYJNSSA-N); Enpiroline (OCVRKDMRZFCUEE-UHFFFAOYSA-N); Ensartinib (GLYMPHUVMRFTFV-QLFBSQMISA-N); Entinostat (INVTYAOGFAGBOE-UHFFFAOYSA-N); Entospletinib (XSMSNFMDVXXHGJ-UHFFFAOYSA-N); Entrectinib (HAYYBYPASCDWEQ-UHFFFAOYSA-N); Enzalutamide (WXCXUHSOUPDCQV-UHFFFAOYSA-N); Enzastaurin (AXRCEOKUDYDWLF-UHFFFAOYSA-N); Epacadostat (FBKMWOJEPMPVTQ-UHFFFAOYSA-N); Eperisone hydrochloride (GTAXGNCCEYZRII-UHFFFAOYSA-N); Epertinib (IBCIAMOTBDGBJN-NRLRZRKLSA-N); Epidoxoform (LNMUJUHEUYMBOT-BCNYZFJLSA-N); Epigallocatechin gallate (WMBWREPUVVBILR-WIYYLYMNSA-N); Epinastine hydrochloride (VKXSGUIOOQPGAF-UHFFFAOYSA-N); Epinephrine (UCTWMZQNUQWSLP-VIFPVBQESA-N); Epirubicin (AOJJSUZBOXZQNB-VTZDEGQISA-N); Epirubicin hydrochloride (MWWSFMDVAYGXBV-FGBSZODSSA-N); Epothilone a (HESCAJZNRMSMJG-KKQRBIROSA-N); Epothilone b (QXRSDHAAWVKZLJ-PVYNADRNSA-N); Epothilone d (XOZIUKBZLSUILX-GIQCAXHBSA-N); Eprazinone dihydrochloride (BPMQVOKMMQFZGV-UHFFFAOYSA-N); Erastin (BKQFRNYHFIQEKN-UHFFFAOYSA-N); Erdafitinib (OLAHOMJCDNXHFI-UHFFFAOYSA-N); Ergotamine tartrate (CJMJLDQKTOJACI-BGQAIRJTSA-N); Eribulin (UFNVPOGXISZXJD-JBQZKEIOSA-N); Erk-IN-3 (PWHIUQBBGPGFFV-GOSISDBHSA-N); Erlotinib (AAKJLRGGTJKAMG-UHFFFAOYSA-N); Erlotinib hydrochloride (GTTBEUCJPZQMDZ-UHFFFAOYSA-N); Erteberel (XIESSJVMWNJCGZ-VKJFTORMSA-N); Erythritol aziridine (COMAQBNTHGBLNQ-OCAPTIKFSA-N); Erythromycin estolate (AWMFUEJKWXESNL-JZBHMOKNSA-N); Erythromycin ethylsuccinate (NSYZCCDSJNWWJL-YXOIYICCSA-N); Erythromycin propionate (TYQXKHPOXXXCTP-CSLYCKPJSA-N); Esculetin (ILEDWLMCKZNDJK-UHFFFAOYSA-N); Estetrol (AJIPIJNNOJSSQC-NYLIRDPKSA-N); Estradiol (VOXZDWNPVJITMN-ZBRFXRBCSA-N); Estradiol acetate (FHXBMXJMKMWVRG-SLHNCBLASA-N); Estradiol benzoate (UYIFTLBWAOGQBI-BZDYCCQFSA-N); Estradiol cypionate (UOACKFBJUYNSLK-XRKIENNPSA-N); Estradiol dipropionate (JQIYNMYZKRGDFK-RUFWAXPRSA-N); Estradiol valerate (RSEPBGGWRJCQGY-RBRWEJTLSA-N); Estramustine (FRPJXPJMRWBBIH-RBRWEJTLSA-N); Estramustine phosphate (ADFOJJHRTBFFOF-RBRWEJTLSA-N); Estramustine phosphate sodium (IIUMCNJTGSMNRO-VVSKJQCTSA-L); Estriol (PROQIPRRNZUXQM-ZXXIGWHRSA-N); Estrone (DNXHEGUUPJUMQT-CBZIJGRNSA-N); Ethacridine lactate (IYLLULUTZPKQBW-UHFFFAOYSA-N); Ethacridine lactate monohydrate (NYEPHMYJRNWPLA-UHFFFAOYSA-N); Ethacrynic acid (AVOLMBLBETYQHX-UHFFFAOYSA-N); Ethanol, 2-(4-[2,2′:6′,2″-terpyridin]-4′-ylphenoxy)-(ITTHDGHSDFMYOE-UHFFFAOYSA-N); Ethenzamide (SBNKFTQSBPKMBZ-UHFFFAOYSA-N); Ethinyl estradiol (BFPYWIDHMRZLRN-SLHNCBLASA-N); Ethionamide (AEOCXXJPGCBFJA-UHFFFAOYSA-N); Ethopropazine hydrochloride (VXPCQISYVPFYRK-UHFFFAOYSA-N); Ethoxyquin (DECIPOUIJURFOJ-UHFFFAOYSA-N); Ethoxzolamide (OUZWUKMCLIBBOG-UHFFFAOYSA-N); Ethyl 1-(3-cyano-3,3-diphenylpropyl)-4-phenylpiperidine-4-carboxylate hydrochloride (SHTAFWKOISOCBI-UHFFFAOYSA-N); Ethyl 3,5-dibromo-4-(3,4-dimethoxyphenyl)-1H-pyrrole-2-carboxylate (OMASOAOYMQBFGC-UHFFFAOYSA-N); Ethyl 4-(1,3-thiazol-2-ylcarbamoylamino)benzoate (OMEMETVMVIWGLI-UHFFFAOYSA-N); Ethyl 4-[(4-chloro-3-oxobutyl)(formyl)amino]benzoate (YTIHVRBBTKXDRM-UHFFFAOYSA-N); Ethyl 5,10,11-trimethylpyrido[3,4-b]carbazole-7-carboxylate (KJPNCIYXIXOPSR-UHFFFAOYSA-N); Ethyl 5,6,11-trimethylpyrido[4,3-b]carbazole-9-carboxylate (MGNHVAFOJUCCIQ-UHFFFAOYSA-N); Ethyl nitrite (QQZWEECEMNQSTG-UHFFFAOYSA-N); Ethyl salicylate (GYCKQBWUSACYIF-UHFFFAOYSA-N); Ethylene dimethanesulfonate (QSQFARNGNIZGAW-UHFFFAOYSA-N); Ethylenimine quinone (RCWJMKCTHJPXJV-UHFFFAOYSA-N); Ethylestrenol (AOXRBFRFYPMWLR-XGXHKTLJSA-N); Ethynodiol diacetate (ONKUMRGIYFNPJW-KIEAKMPYSA-N); Etofenamate (XILVEPYQJIOVNB-UHFFFAOYSA-N); Etofylline (NWPRCRWQMGIBOT-UHFFFAOYSA-N); Etoglucid (UMILHIMHKXVDGH-UHFFFAOYSA-N); Etoposide (VJJPUSNTGOMMGY-MRVIYFEKSA-N); Etretinate (HQMNCQVAMBCHCO-DJRRULDNSA-N); Eupenifeldin (BWTQHPFSWXJOGP-YLLNCWMYSA-N); Evacetrapib (IHIUGIVXARLYHP-UXNJHFGPSA-N); Everolimus (HKVAMNSJSFKALM-GKUWKFKPSA-N); Evofosfamide (UGJWRPJDTDGERK-UHFFFAOYSA-N); Exatecan (ZVYVPGLRVWUPMP-FYSMJZIKSA-N); Exemestane (BFYIZQONLCFLEV-DAELLWKTSA-N); Exifone (XEDWWPGWIXPVRQ-UHFFFAOYSA-N); Ezatiostat (GWEJFLVSOGNLSS-WPFOTENUSA-N); Ezetimibe (OLNTVTPDXPETLC-XPWALMASSA-N); FDA 0090 (LXZOLFWCOGHEOF-UHFFFAOYSA-N); FN-1501 (VXLAKHWYGRKCGI-UHFFFAOYSA-N); Fadraciclib (DLPIYBKBHMZCJI-WBVHZDCISA-N); Fadrozole (CLPFFLWZZBQMAO-UHFFFAOYSA-N); Falnidamol (FTFRZXFNZVCRSK-UHFFFAOYSA-N); Famotidine (XUFQPHANEAPEMJ-UHFFFAOYSA-N); Fascaplysine (PWUOOJVYZQILBG-UHFFFAOYSA-N); Fasiglifam (BZCALJIHZVNMGJ-HSZRJFAPSA-N); Fasudil (NGOGFTYYXHNFQH-UHFFFAOYSA-N); Febantel (HMCCXLBXIJMERM-UHFFFAOYSA-N); Febuxostat (BQSJTQLCZDPROO-UHFFFAOYSA-N); Fedratinib (JOOXLOJCABQBSG-UHFFFAOYSA-N); Felbamate (WKGXYQFOCVYPAC-UHFFFAOYSA-N); Felodipine (RZTAMFZIAATZDJ-UHFFFAOYSA-N); Fenbendazole (HDDSHPAODJUKPD-UHFFFAOYSA-N); Fenipentol (OVGORFFCBUIFIA-UHFFFAOYSA-N); Fenofibrate (YMTINGFKWWXKFG-UHFFFAOYSA-N); Fenoldopam (TVURRHSHRRELCG-UHFFFAOYSA-N); Fenoverine (UBAJTZKNDCEGKL-UHFFFAOYSA-N); Fenretinide (AKJHMTWEGVYYSE-FXILSDISSA-N); Fentanyl (PJMPHNIQZUBGLI-UHFFFAOYSA-N); Fenticonazole (ZCJYUTQZBAIHBS-UHFFFAOYSA-N); Feprazone (RBBWCVQDXDFISW-UHFFFAOYSA-N); Ferric-taxol (DENRCSJSKWORLT-NOSCCTPQSA-N); Filanesib (LLXISKGBWFTGEI-FQEVSTJZSA-N); Filgotinib (RIJLVEAXPNLDTC-UHFFFAOYSA-N); Fimepinostat (JOWXJLIFIIOYMS-UHFFFAOYSA-N); Finasteride (DBEPLOCGEIEOCV-WSBQPABSSA-N); Fingolimod (KKGQTZUTZRNORY-UHFFFAOYSA-N); Fingolimod hydrochloride (SWZTYAVBMYWFGS-UHFFFAOYSA-N); Fipexide hydrochloride (MVOWQBJZZKOQNU-UHFFFAOYSA-N); Fipronil (ZOCSXAVNDGMNBV-UHFFFAOYSA-N); Fisetin (XHEFDIBZLJXQHF-UHFFFAOYSA-N); Flavone (VHBFFQKBGNRLFZ-UHFFFAOYSA-N); Flavopiridol hydrochloride (LGMSNQNWOCSPIK-LWHGMNCYSA-N); Flecainide (DJBNUMBKLMJRSA-UHFFFAOYSA-N); Florfenicol (AYIRNRDRBQJXIF-NXEZZACHSA-N); Floxuridine (ODKNJVUHOIMIIZ-RRKCRQDMSA-N); Flubendazole (CPEUVMUXAHMANV-UHFFFAOYSA-N); Flucytosine (XRECTZIEBJDKEO-UHFFFAOYSA-N); Fludara (GIUYCYHIANZCFB-UHFFFAOYSA-N); Fludarabine (HBUBKKRHXORPQB-FJFJXFQQSA-N); Fludarabine phosphate (GIUYCYHIANZCFB-FJFJXFQQSA-N); Flumatinib (BJCJYEYYYGBROF-UHFFFAOYSA-N); Flumazenil (OFBIFZUFASYYRE-UHFFFAOYSA-N); Flumequine (DPSPPJIUMHPXMA-UHFFFAOYSA-N); Flumethasone (WXURHACBFYSXBI-GQKYHHCASA-N); Flunarizine (SMANXXCATUTDDT-QPJJXVBHSA-N); Flunisolide (XSFJVAJPIHIPKU-XWCQMRHXSA-N); Fluocinolone acetonide (FEBLZLNTKCEFIT-VSXGLTOVSA-N); Fluocinonide (WJOHZNCJWYWUJD-IUGZLZTKSA-N); Fluorocyclopentenylcytosine (QLLGKCJUPWYJON-HLTSFMKQSA-N); Fluorometholone (FAOZLTXFLGPHNG-KNAQIMQKSA-N); Fluorometholone acetate (YRFXGQHBPBMFHW-SBTZIJSASA-N); Fluorouracil (GHASVSINZRGABV-UHFFFAOYSA-N); Fluoxetine (RTHCYVBBDHJXIQ-UHFFFAOYSA-N); Fluoxetine hydrochloride (GIYXAJPCNFJEHY-UHFFFAOYSA-N); Fluoxymesterone (YLRFCQOZQXIBAB-RBZZARIASA-N); Flupentixol (NJMYODHXAKYRHW-DVZOWYKESA-N); Fluphenazine (PLDUPXSUYLZYBN-UHFFFAOYSA-N); Flupirtine (JUUFBMODXQKSTD-UHFFFAOYSA-N); Flupirtine maleate (DPYIXBFZUMCMJM-BTJKTKAUSA-N); Flurazepam (SAADBVWGJQAEFS-UHFFFAOYSA-N); Flurbiprofen (SYTBZMRGLBWNTM-UHFFFAOYSA-N); Fluspirilene (QOYHHIBFXOOADH-UHFFFAOYSA-N); Flutamide (MKXKFYHWDHIYRV-UHFFFAOYSA-N); Fluticasone propionate (WMWTYOKRWGGJOA-CENSZEJFSA-N); Fluvastatin (FJLGEFLZQAZZCD-MCBHFWOFSA-N); Fluvastatin sodium (ZGGHKIMDNBDHJB-NRFPMOEYSA-M); Fluvastatine (FJLGEFLZQAZZCD-JUFISIKESA-N); Fluvoxamine maleate (LFMYNZPAVPMEGP-PIDGMYBPSA-N); Folic acid (OVBPIULPVIDEAO-LBPRGKRZSA-N); Fomepizole (RIKMMFOAQPJVMX-UHFFFAOYSA-N); Foretinib (CXQHYVUVSFXTMY-UHFFFAOYSA-N); Formerly NSC 364574 (BVGDPXVMSLWVOD-HZMRJVDMSA-N); Formestane (OSVMTWJCGUFAOD-KZQROQTASA-N); Fosbretabulin disodium (VXNQMUVMEIGUJW-XNOMRPDFSA-L); Fostamatinib (GKDRMWXFWHEQQT-UHFFFAOYSA-N); Fpjworgegi (UCJZOKGUEJUNIO-IINYFYTJSA-N); From dolabella auricularia (sea hare) (OFDNQWIFNXBECV-MOFABKIVSA-N); From marine animal axinella sp. (LMZKVILFCFSDGK-QTBWQIHVSA-N); From marine animal, axinella sp. (FXNFULJVOQMBCW-YAWMHOIFSA-N); Fruquintinib (BALLNEJQLSTPIO-UHFFFAOYSA-N); Fulvestrant (VWUXBMIQPBEWFH-WCCTWKNTSA-N); Fumagillin (NGGMYCMLYOUNGM-CSDLUJIJSA-N); Furoxone (PLHJDBGFXBMTGZ-WEVVVXLNSA-N); Fursultiamine hydrochloride (OPGOLNDOMSBSCW-CLNHMMGSSA-N); Futibatinib (KEIPNCCJPRMIAX-HNNXBMFYSA-N); GDC-0425 (XEZLBMHDUXSICI-UHFFFAOYSA-N); GDC-0575 (BAZRWWGASYWYGB-SNVBAGLBSA-N); GDC-0927 (KJAAPZIFCQQQKX-NDEPHWFRSA-N); GS-9901 (XDSXYMOZKDUASY-INIZCTEOSA-N); GSK-923295 (WHMXDBPHBVLYRC-OFVILXPXSA-N); GSK1070916 (QTBWCSQGBMPECM-UHFFFAOYSA-N); GSK126 (FKSFKBQGSFSOSM-QFIPXVFZSA-N); GSK1292263 (AYJRTVVIBJSSKN-UHFFFAOYSA-N); GSK1838705A (HZTYDQRUAWIZRE-UHFFFAOYSA-N); GSK256066 (JFHROPTYMMSOLG-UHFFFAOYSA-N); GSK2636771 (XTKLTGBKIDQGQL-UHFFFAOYSA-N); GSK3145095 (ATQAGKAMBISZQM-HNNXBMFYSA-N); GSK461364 (ZHJGWYRLJUCMRT-QGZVFWFLSA-N); GW0742 (HWVNEWGKWRGSRK-UHFFFAOYSA-N); Gadodiamide (XPCLDSMKWNNKOM-UHFFFAOYSA-K); Galantamine hydrobromide (QORVDGQLPPAFRS-XPSHAMGMSA-N); Galeterone (PAFKTGFSEFKSQG-PAASFTFBSA-N); Gallamine triethiodide (REEUVFCVXKWOFE-UHFFFAOYSA-K); Gallic acid (LNTHITQWFMADLM-UHFFFAOYSA-N); Gallotannin (LRBQNJMCXXYXIU-PPKXGCFTSA-N); Galunisertib (IVRXNBXKWIJUQB-UHFFFAOYSA-N); Ganciclovir (IRSCQMHQWWYFCW-UHFFFAOYSA-N); Gandotinib (SQSZANZGUXWJEA-UHFFFAOYSA-N); Ganetespib (RVAQIUULWULRNW-UHFFFAOYSA-N); Gartisertib (QAYHKBLKSXWOEO-UHFFFAOYSA-N); Gdc-0084 (LGWACEZVCMBSKW-UHFFFAOYSA-N); Gdc-0152 (WZRFLSDVFPIXOV-LRQRDZAKSA-N); Gdc-0349 (RGJOJUGRHPQXGF-INIZCTEOSA-N); Gdc-0623 (RFWVETIZUQEJEF-UHFFFAOYSA-N); Gedatolisib (DWZAEMINVBZMHQ-UHFFFAOYSA-N); Gefarnate (ZPACYDRSPFRDHO-ROBAGEODSA-N); Gefitinib (XGALLCVXEZPNRQ-UHFFFAOYSA-N); Geldanamycin (QTQAWLPCGQOSGP-KSRBKZBZSA-N); Geldanamycin (QTQAWLPCGQOSGP-PHLMVCJGSA-N); Geldanamycin (QTQAWLPCGQOSGP-VKNBREQOSA-N); GemMP[10] ion (YGWQAZCOAPWRBC-UHFFFAOYSA-W) salts; Gemcitabine (SDUQYLNIPVEERB-QPPQHZFASA-N); Gemcitabine elaidate (HESSNRGIEVBPRB-QDDPNBLJSA-N); Gemcitabine hydrochloride (OKKDEIYWILRZIA-OSZBKLCCSA-N); Gemfibrozil (HEMJJKBWTPKOJG-UHFFFAOYSA-N); Genistein (TZBJGXHYKVUXJN-UHFFFAOYSA-N); Gentian violet (ZXJXZNDDNMQXFV-UHFFFAOYSA-M); Gentian violet b (JFTBTTPUYRGXDG-UHFFFAOYSA-N); Gentian violet cation ion (LGLFFNDHMLKUMI-UHFFFAOYSA-N) salts; Genz-644282 (BAORCAMWLWRZQG-UHFFFAOYSA-N); Gestodene (SIGSPDASOTUPFS-XUDSTZEESA-N); Gestrinone (BJJXHLWLUDYTGC-ANULTFPQSA-N); Gilteritinib (GYQYAJJFPNQOOW-UHFFFAOYSA-N); Gimatecan (UIVFUQKYVFCEKJ-OPTOVBNMSA-N); Gimeracil (ZPLQIPFOCGIIHV-UHFFFAOYSA-N); Ginkgolide b (SQOJOAFXDQDRGF-UHFFFAOYSA-N); Giredestrant (GQCXHIKRWBIQMD-AKJBCIBTSA-N); Givinostat (YALNUENQHAQXEA-UHFFFAOYSA-N); Givinostat hydrochloride monohydrate (FKGKZBBDJSKCIS-UHFFFAOYSA-N); Glafenine (GWOFUCIGLDBNKM-UHFFFAOYSA-N); Glafenine hydrochloride (CEUMONXVSJOJIH-UHFFFAOYSA-N); Glaucarubinone (WRBGCYVAJRRQKP-STDAJNJZSA-N); Glesatinib (YRCHYHRCBXNYNU-UHFFFAOYSA-N); Gliclazide (BOVGTQGAOIONJV-UHFFFAOYSA-N); Gliquidone (LLJFMFZYVVLQKT-UHFFFAOYSA-N); Glutamic acid,4-diamino-6-pteridinyl)methyl]methylamino]benzoyl]-(RVHHUTDEYCUBRL-UHFFFAOYSA-N); Glycopyrrolate (VPNYRYCIDCJBOM-UHFFFAOYSA-M); Glyoxylic acid, (p-tolylsulfonyl)hydrazone (TUYHVIQNWSSXND-UXBLZVDNSA-N); Go-6976 (VWVYILCFSYNJHF-UHFFFAOYSA-N); Golidocitinib (CVCVOSPZEVINRM-MRXNPFEDSA-N); Golvatinib (UQRCJCNVNUFYDX-UHFFFAOYSA-N); Gossypol (QBKSWRVVCFFDOT-UHFFFAOYSA-N); Gossypol acetic acid (NIOHNDKHQHVLKA-UHFFFAOYSA-N); Gramicidina (ZWCXYZRRTRDGQE-SORVKSEFSA-N); Granisetron (MFWNKCLOYSRHCJ-UHFFFAOYSA-N); Griffipavixanthone (KAPVRSRPLHVWCH-JNFGOLMQSA-N); Griseofulvin (DDUHZTYCFQRHIY-RBHXEPJQSA-N); Guaiacol carbonate (ORUJFMPWKPVXLZ-UHFFFAOYSA-N); Guaifenesin (HSRJKNPTNIJEKV-UHFFFAOYSA-N); Guanazole (PKWIYNIDEDLDCJ-UHFFFAOYSA-N); Guanfacine hydrochloride (DGFYECXYGUIODH-UHFFFAOYSA-N); Guanoxan (HIUVKVDQFXDZHU-UHFFFAOYSA-N); Gusacitinib (NLFLXLJXEIUQDL-UHFFFAOYSA-N); Gusperimus (IDINUJSAMVOPCM-UHFFFAOYSA-N); Gusperimus monohydrochloride (FODMSVBVCPOQRL-UHFFFAOYSA-N); H-xiHyp-DL-N(Me)Leu-DL-xiThr(1)-Unk-Leu-Pro-N(Me)Tyr(Me)-(1) (DCKHYMCBBYLYHE-ZMRKGEOASA-N); H3B-6527 (MBWRLLRCTIYXDW-UHFFFAOYSA-N); HMS3653G06 (XBJVUZASONIJRS-UHFFFAOYSA-N); Halofantrine (FOHHNHSLJDZUGQ-UHFFFAOYSA-N); Halofantrine hydrochloride (WANGFTDWOFGECH-UHFFFAOYSA-N); Halomonoterpene (OVLCIYBVQSJPKK-SCZZXKLOSA-N); Haloperidol (LNEPOXFFQSENCJ-UHFFFAOYSA-N); Haloprogin (CTETYYAZBPJBHE-UHFFFAOYSA-N); Haloquinone (CPYFLMXPZMBECD-UHFFFAOYSA-N); Haloxon (KULDXINYXFTXMO-UHFFFAOYSA-N); Harmine (BXNJHAXVSOCGBA-UHFFFAOYSA-N); Harringtonine (HAVJATCHLFRDHY-KSZYUSJVSA-N); Hdac inhibitor CHR-3996 (QRGHOAATPOLDPF-BYICEURKSA-N); Hedamycin (RZOFHOWMWMTHDX-UHFFFAOYSA-N); Hellebrin (DCSLTSSPIJWEJN-UHFFFAOYSA-N); Heroin (GVGLGOZIDCSQPN-PVHGPHFFSA-N); Hesperidin (QUQPHWDTPGMPEX-QJBIFVCTSA-N); Hexachlorophene (ACGUYXCXAPNIKK-UHFFFAOYSA-N); Hexafluorenium bromide (WDEFPRUEZRUYNW-UHFFFAOYSA-L); Hexanoic acid, 6-[(7-methoxy-1-nitro-9-acridinyl)amino]-(DMVRDIUGDIVKPU-UHFFFAOYSA-N); Hexestrol (PBBGSZCBWVPOOL-HDICACEKSA-N); Hexylresorcinol (WFJIVOKAWHGMBH-UHFFFAOYSA-N); Histamine (NTYJJOPFIAHURM-UHFFFAOYSA-N); Hitachimycin (PLQKHNPZPRTISL-KRZXCKFYSA-N); Hmn-214 (OCKHRKSTDPOHEN-BQYQJAHWSA-N); Hofmann's violet (PHWHSHNIIPVVHF-UHFFFAOYSA-N); Holacanthone (OFHVBIQKCNMHKC-FJSLBGAUSA-N); Homidium bromide (ZMMJGEGLRURXTF-UHFFFAOYSA-N); Honokiol (FVYXIJYOAGAUQK-UHFFFAOYSA-N); Hydergine (UOOWRCRLTSXSAV-GSZJWLEYSA-N); Hydralazine hydrochloride (ZUXNZUWOTSUBMN-UHFFFAOYSA-N); Hydramycin (WZNWUDKIQDIRSR-UHFFFAOYSA-N); Hydrazinecarbothioamide, 2-(2-pyridinylmethylene)-N-(2-pyridinyl)methyl-(BTFYMPJMYCNLRM-LICLKQGHSA-N); Hydrazinecarbothioamide, 2-[(5-amino-1-isoquinolinyl)methylene]-(MLTULNASULCZLR-UUASQNMZSA-N); Hydrazinecarboximidamide, 2-[(4-bromophenyl)phenylmethylene]-N-methyl-(SOFUQTGDLLAPQE-XMHGGMMESA-N); Hydrocortamate (FWFVLWGEFDIZMJ-FOMYWIRZSA-N); Hydrocortisone (JYGXADMDTFJGBT-VWUMJDOOSA-N); Hydrocortisone acetate (ALEXXDVDDISNDU-JZYPGELDSA-N); Hydrocortisone butyrate (BMCQMVFGOVHVNG-TUFAYURCSA-N); Hydrocortisone cypionate (DLVOSEUFIRPIRM-KAQKJVHQSA-N); Hydrocortisone probutate (FOGXJPFPZOHSQS-AYVLZSQQSA-N); Hydrocortisone sodium succinate (HHZQLQREDATOBM-CODXZCKSSA-M); Hydrofluoric acid (KRHYYFGTRYWZRS-UHFFFAOYSA-N); Hydroquinone (QIGBRXMKCJKVMJ-UHFFFAOYSA-N); Hydroxy AT 125 (KUYDILDQEMQBKJ-ZVHKOUPVSA-N); Hydroxychloroquine (XXSMGPRMXLTPCZ-UHFFFAOYSA-N); Hydroxychloroquine sulfate (JCBIVZZPXRZKTI-UHFFFAOYSA-N); Hydroxyflutamide (YPQLFJODEKMJEF-UHFFFAOYSA-N); Hydroxyprogesterone caproate (DOMWKUIIPQCAJU-LJHIYBGHSA-N); Hydroxyurea (VSNHCAURESNICA-UHFFFAOYSA-N); Hydroxyzine (ZQDWXGKKHFNSQK-UHFFFAOYSA-N); Hymecromone (HSHNITRMYYLLCV-UHFFFAOYSA-N); Hypericin (YDOIFHVUBCIUHF-UHFFFAOYSA-N); Hypoxanthine (FDGQSTZJBFJUBT-UHFFFAOYSA-N); ICG-001 (HQWTUOLCGKIECB-XZWHSSHBSA-N); INCB-057643 (VZSAMEOETVNDQH-UHFFFAOYSA-N); INCB054329 (XYLPKCDRAAYATL-OAHLLOKOSA-N); IPF4CM70ZI (BGMKTSYQDUAZFJ-QFIPXVFZSA-N); ITD-1 (ULFUJLFTRWWLPO-UHFFFAOYSA-N); Iberdomide (IXZOHGPZAQLIBH-NRFANRHFSA-N); Ibrutinib (XYFPWWZEPKGCCK-GOSISDBHSA-N); Ibudilast (ZJVFLBOZORBYFE-UHFFFAOYSA-N); Ibuprofen (HEFNNWSXXWATRW-UHFFFAOYSA-N); Ibutamoren (UMUPQWIGCOZEOY-JOCHJYFZSA-N); Icaritin (TUUXBSASAQJECY-UHFFFAOYSA-N); Icatibant (QURWXBZNHXJZBE-SKXRKSCCSA-N); Icotinib (QQLKULDARVNMAL-UHFFFAOYSA-N); Idarubicin (XDXDZDZNSLXDNA-TZNDIEGXSA-N); Idarubicin hydrochloride (JVHPTYWUBOQMBP-RVFAQHLVSA-N); Idasanutlin (TVTXCJFHQKSQQM-LJQIRTBHSA-N); Idazoxan (HPMRFMKYPGXPEP-UHFFFAOYSA-N); Idebenone (JGPMMRGNQUBGND-UHFFFAOYSA-N); Idelalisib (IFSDAJWBUCMOAH-HNNXBMFYSA-N); Idoxifene (JJKOTMDDZAJTGQ-DQSJHHFOSA-N); Idoxuridine (XQFRJNBWHJMXHO-RRKCRQDMSA-N); Idronoxil (ZZUBHVMHNVYXRR-UHFFFAOYSA-N); Ifenprodil (UYNVMODNBIQBMV-UHFFFAOYSA-N); Ifosfamide (HOMGKSMUEGBAAB-UHFFFAOYSA-N); Illudin m (QVMDIQLUNODCTG-OCCSQVGLSA-N); Illudin s (DDLLIYKVDWPHJI-RDBSUJKOSA-N); Iloperidone (XMXHEBAFVSFQEX-UHFFFAOYSA-N); Iloprost (HIFJCPQKFCZDDL-ACWOEMLNSA-N); Ilorasertib (WPHKIQPVPYJNAX-UHFFFAOYSA-N); Imatinib (KTUFNOKKBVMGRW-UHFFFAOYSA-N); Imatinib mesylate (YLMAHDNUQAMNNX-UHFFFAOYSA-N); Imexon (BIXBBIPTYBJTRY-UHFFFAOYSA-N); Imipenem (ZSKVGTPCRGIANV-ZXFLCMHBSA-N); Imipramine (BCGWQEUPMDMJNV-UHFFFAOYSA-N); Imiquimod (DOUYETYNHWVLEO-UHFFFAOYSA-N); Imperatorin (OLOOJGVNMBJLLR-UHFFFAOYSA-N); Inavolisib (SGEUNORSOZVTOL-CABZTGNLSA-N); Incyclinide (NBRQRXRBIHVLGI-OWXODZSWSA-N); Indapamide (NDDAHWYSQHTHNT-UHFFFAOYSA-N); Indibulin (SOLIIYNRSAWTSQ-UHFFFAOYSA-N); Indicine N-oxide (DNAWGBOKUFFVMB-PZIGJSDBSA-N); Indinavir sulfate (NUBQKPWHXMGDLP-BDEHJDMKSA-N); Indirubin (JNLNPCNGMHKCKO-UHFFFAOYSA-N); Indisulam (SETFNECMODOHTO-UHFFFAOYSA-N); Indium chloride (InCl3) (PSCMQHVBLHHWTO-UHFFFAOYSA-K); Indocyanine green (MOFVSTNWEDAEEK-UHFFFAOYSA-M); Indole-3-carbinol (IVYPNXXAYMYVSP-UHFFFAOYSA-N); Indomethacin (CGIGDMFJXJATDK-UHFFFAOYSA-N); Indoprofen (RJMIEHBSYVWVIN-UHFFFAOYSA-N); Indoramin hydrochloride (AFJSFHAKSSWOKG-UHFFFAOYSA-N); Indoximod (ZADWXFSZEAPBJS-SNVBAGLBSA-N); Infigratinib (QADPYRIHXKWUSV-UHFFFAOYSA-N); Infigratinib phosphate (GUQNHCGYHLSITB-UHFFFAOYSA-N); Ingenol mebutate (VDJHFHXMUKFKET-WDUFCVPESA-N); Iniparib (MDOJTZQKHMAPBK-UHFFFAOYSA-N); Inobrodib (SKDNDJWEBPQKCS-RIQBOWGZSA-N); Inosine dialdehyde (RGWOFTGZWJGPHG-NKWVEPMBSA-N); Inositol (CDAISMWEOUEBRE-UHFFFAOYSA-N); Iobenguane sulfate (XNACDNPGABUBFR-UHFFFAOYSA-N); Iodoacetamide (PGLTVOMIXTUURA-UHFFFAOYSA-N); Iodoform (OKJPEAGHQZHRQV-UHFFFAOYSA-N); Iodol (VJOVAKSZILJDBB-UHFFFAOYSA-N); Iodonium, dimesityl-chloride ion (WAKCRSHUCNABAR-UHFFFAOYSA-N) salts; Iodoquinol (UXZFQZANDVDGMM-UHFFFAOYSA-N); Iomazenil (FRIZVHMAECRUBR-UHFFFAOYSA-N); Ipatasertib (GRZXWCHAXNAUHY-NSISKUIASA-N); Ipratropium bromide hydrate (KEWHKYJURDBRMN-XFQAGIBXSA-M); Ipriflavone (SFBODOKJTYAUCM-UHFFFAOYSA-N); Irinotecan (UWKQSNNFCGGAFS-XIFFEERXSA-N); Irinotecan hydrochloride (GURKHSYORGJETM-WAQYZQTGSA-N); Irinotecan hydrochloride trihydrate (KLEAIHJJLUAXIQ-JDRGBKBRSA-N); Irofulven (NICJCIQSJJKZAH-AWEZNQCLSA-N); Iron Chelator, Dp44mT (XOBIGRNRXCAMJQ-UHFFFAOYSA-N); Isobaccharin (DGBITFNXKQHKLI-CYLYTYKUSA-N); Isoconazole (MPIPASJGOJYODL-UHFFFAOYSA-N); Isoetharine mesylate (SOYAGMVKMXZVNZ-UHFFFAOYSA-N); Isoevodiamine (TXDUTHBFYKGSAH-UHFFFAOYSA-N); Isoflupredone acetate (ZOCUOMKMBMEYQV-GSLJADNHSA-N); Isoliquiritigenin (DXDRHHKMWQZJHT-FPYGCLRLSA-N); Isoniazid (QRXWMOHMRWLFEY-UHFFFAOYSA-N); Isopropamide ion (JTPUMZTWMWIVPA-UHFFFAOYSA-O) salts; Isopropyl unoprostone (XXUPXHKCPIKWLR-JHUOEJJVSA-N); Isoproterenol (JWZZKOKVBUJMES-UHFFFAOYSA-N); Isoquercetin (OVSQVDMCBVZWGM-QSOFNFLRSA-N); Isoreserpin (QEVHRUUCFGRFIF-UHFFFAOYSA-N); Isosulfan blue (NLUFDZBOHMOBOE-UHFFFAOYSA-M); Isothiazolo[4,3-(beta-D-ribofuranosyl)-(YGFGQYZYMXRMTP-UHFFFAOYSA-N); Isotic (AQSYHEKLGWEVDF-UHFFFAOYSA-N); Isotretinoin (SHGAZHPCJJPHSC-XFYACQKRSA-N); Ispinesib (QJZRFPJCWMNVAV-HHHXNRCGSA-N); Isradipine (HMJIYCCIJYRONP-UHFFFAOYSA-N); Istodax (OHRURASPPZQGQM-GCCNXGTGSA-N); Itasetron (RWXRJSRJIITQAK-YOGCLGLASA-N); Itraconazole (VHVPQPYKVGDNFY-ZPGVKDDISA-N); Itraconazolum [latin](VHVPQPYKVGDNFY-UHFFFAOYSA-N); Ivabradine hydrochloride (HLUKNZUABFFNQS-ZMBIFBSDSA-N); Ivacaftor (PURKAOJPTOLRMP-UHFFFAOYSA-N); Ivermectin B1a (AZSNMRSAGSSBNP-XPNPUAGNSA-N); Ivosidenib (WIJZXSAJMHAVGX-DHLKQENFSA-N); Ixabepilone (FABUFPQFXZVHFB-PVYNADRNSA-N); Ixazomib (MXAYKZJJDUDWDS-LBPRGKRZSA-N); JI-101 (ZXBFYBLSJMEBEP-UHFFFAOYSA-N); JNJ-7706621 (KDKUVYLMPJIGKA-UHFFFAOYSA-N); JQ-35, (S)-(PKQXLRYFPSZKDU-QFIPXVFZSA-N); JTC-801 free base (VTGBZWHPJFMTKS-UHFFFAOYSA-N); Janex-1 (HOZUXBLMYUPGPZ-UHFFFAOYSA-N); Janus green b (XXACTDWGHQXLGW-UHFFFAOYSA-M); Jasplakinolide (GQWYWHOHRVVHAP-DHKPLNAMSA-N); Jnj-38877605 (JRWCBEOAFGHNNU-UHFFFAOYSA-N); KPT-185 (NLNGWFLRRRYNIL-PLNGDYQASA-N); KW-2478 free base (VFUXSYAXEKYYMB-UHFFFAOYSA-N); Kaempferol (IYRMWMYZSQPJKC-UHFFFAOYSA-N); Kalafungin (XUWPJKDMEZSVTP-UHFFFAOYSA-N); Karenitecin (POADTFBBIXOWFJ-VWLOTQADSA-N); Kazusamycin a (KZMHNEBMQDBQND-MRBODPGGSA-N); Kendomycin (HKLDUJXJTQJSEJ-OLXNOMCWSA-N); Kenpaullone (QQUXFYAWXPMDOE-UHFFFAOYSA-N); Ketanserin (FPCCSQOGAWCVBH-UHFFFAOYSA-N); Ketoconazole (XMAYWYJOQHXEEK-OZXSUGGESA-N); Ketoprofen (DKYWVDODHFEZIM-UHFFFAOYSA-N); Ketorolac (OZWKMVRBQXNZKK-UHFFFAOYSA-N); Ketotifen (ZCVMWBYGMWKGHF-UHFFFAOYSA-N); Kinamycin c (MXDLFLPONIABIS-OLKYXYMISA-N); Ku-0063794 (RFSMUFRPPYDYRD-CALCHBBNSA-N); L-Chlorozotocin (MKQWTWSXVILIKJ-UHFFFAOYSA-N); L-Cysteine, S-(2-naphthalenyldiphenylmethyl)-(BCBSYXGYLMMICN-UHFFFAOYSA-N); L-Cysteine, S-[(4-chlorophenyl)diphenylmethyl]-(BSTXYLCINUXYQF-UHFFFAOYSA-N); L-Cysteine, S-[(4-fluorophenyl)diphenylmethyl]-(GYNSTRCJYLZSGY-UHFFFAOYSA-N); L-Cysteine, S-[bis(4-methylphenyl)phenylmethyl]-(MSPIKMKQZSFDCR-UHFFFAOYSA-N); L-Name (KCWZGJVSDFYRIX-YFKPBYRVSA-N); LCL161 (UFPFGVNKHCLJJO-SSKFGXFMSA-N); LDN-193189 monohydrochloride (PCCDKTWDGDFRME-UHFFFAOYSA-N); LMP744 (QCSDJDQOJBQTDV-UHFFFAOYSA-N); LSZ-102 (SJXNPGGVGZXKKI-NYYWCZLTSA-N); LX7101 (PWPNYABQEOGNNC-UHFFFAOYSA-N); LY-2874455 (GKJCVYLDJWTWQU-CXLRFSCWSA-N); LY2090314 (HRJWTAWVFDCTGO-UHFFFAOYSA-N); LY2584702 (FYXRSVDHGLUMHB-UHFFFAOYSA-N); LY3009120 (HHCBMISMPSAZBF-UHFFFAOYSA-N); LY3295668 (YQQZZYYQTCPEAS-OYLFLEFRSA-N); Lacidipine (GKQPCPXONLDCMU-CCEZHUSRSA-N); Lambdamycin (PONPPNYZKHNPKZ-UHFFFAOYSA-N); Lamivudine (JTEGQNOMFQHVDC-NKWVEPMBSA-N); Lamotrigine (PYZRQGJRPPTADH-UHFFFAOYSA-N); Lanatoside c (JAYAGJDXJIDEKI-PTGWOZRBSA-N); Landomycin a (YMSZNAXJMXNNPT-OELAXGSHSA-N); Lansoprazole (MJIHNNLFOKEZEW-UHFFFAOYSA-N); Lapatinib (BCFGMOOMADDAQU-UHFFFAOYSA-N); Lapatinib ditosylate (UWYXLGUQQFPJRI-UHFFFAOYSA-N); Laquinimod (GKWPCEFFIHSJOE-UHFFFAOYSA-N); Larotrectinib (NYNZQNWKBKUAII-KBXCAEBGSA-N); Lasofoxifene (GXESHMAMLJKROZ-IAPPQJPRSA-N); Latrepirdine (JNODQFNWMXFMEV-UHFFFAOYSA-N); Lauryl pyridinium 5-chloro-2-mercaptobenzothiazole (NPUUGQIQOUWACG-UHFFFAOYSA-M); Lead, tripropyl-p-tolylsulfonamido-(GNJBZWAHEQMOJF-UHFFFAOYSA-N); Ledipasvir (GS5885) (PIAIRSYMCIVYSX-KMWAZVGDSA-N); Leflunomide (VHOGYURTWQBHIL-UHFFFAOYSA-N); Leinamycin (ZHTRILQJTPJGNK-FYBAATNNSA-N); Lenalidomide (GOTYRUGSSMKFNF-UHFFFAOYSA-N); Lenvatinib (WOSKHXYHFSIKNG-UHFFFAOYSA-N); Lercanidipine (ZDXUKAKRHYTAKV-UHFFFAOYSA-N); Lercanidipine hydrochloride (WMFYOYKPJLRMJI-UHFFFAOYSA-N); Lestaurtinib (UIARLYUEJFELEN-LROUJFHJSA-N); Letrozole (HPJKCIUCZWXJDR-UHFFFAOYSA-N); Leucovorin (VVIAGPKUTFNRDU-ABLWVSNPSA-N); Leurocristine (OGWKCGZFUXNPDA-CFWMRBGOSA-N); Levallorphan (OZYUPQUCAUTOBP-QXAKKESOSA-N); Levamisole hydrochloride (LAZPBGZRMVRFKY-HNCPQSOCSA-N); Levetiracetam (HPHUVLMMVZITSG-LURJTMIESA-N); Levodopa (WTDRDQBEARUVNC-LURJTMIESA-N); Levomepromazine maleate (IFLZPECPTYCEBR-VIEYUMQNSA-N); Levonordefrin (GEFQWZLICWMTKF-CDUCUWFYSA-N); Levosimendan (WHXMKTBCFHIYNQ-SECBINFHSA-N); Levothyroxine (XUIIKFGFIJCVMT-LBPRGKRZSA-N); Lexibulin (MTJHLONVHHPNSI-IBGZPJMESA-N); Licofelone (UAWXGRJVZSAUSZ-UHFFFAOYSA-N); Lidoflazine (ZBIAKUMOEKILTF-UHFFFAOYSA-N); Lillyl 99094 (YNWLWZCSXNAXCM-UHFFFAOYSA-N); Linagliptin (LTXREWYXXSTFRX-QGZVFWFLSA-N); Lindane (JLYXXMFPNIAWKQ-UHFFFAOYSA-N); Linezolid (TYZROVQLWOKYKF-ZDUSSCGKSA-N); Linifanib (MPVGZUGXCQEXTM-UHFFFAOYSA-N); Linolenic acid (DTOSIQBPPRVQHS-PDBXOOCHSA-N); Linsitinib (PKCDDUHJAFVJJB-UHFFFAOYSA-N); Liothyronine (AUYYCJSJGJYCDS-LBPRGKRZSA-N); Liranaftate (VPHPQNGOVQYUMG-UHFFFAOYSA-N); Lisavanbulin (NIPZLALJRAHABJ-IBGZPJMESA-N); Lisuride (BKRGVLQUQGGVSM-KBXCAEBGSA-N); Litronesib (YVAFBXLHPINSIK-QHCPKHFHSA-N); Lixivaptan (PPHTXRNHTVLQED-UHFFFAOYSA-N); Lobaplatin ion (XSMVECZRZBFTIZ-UHFFFAOYSA-M) salts; Lobeline (MXYUKLILVYORSK-HBMCJLEFSA-N); Lobeline hcl (MKMYPTLXLWOUSO-NFQNBQCWSA-N); Lofepramine hydrochloride (ZWZIQPOLMDPIQM-UHFFFAOYSA-N); Lomerizine (JQSAYKKFZOSZGJ-UHFFFAOYSA-N); Lomerizine hydrochloride (LOGVKVSFYBBUAJ-UHFFFAOYSA-N); Lometrexol (ZUQBAQVRAURMCL-DOMZBBRYSA-N); Lomibuvir (WPMJNLCLKAKMLA-UHFFFAOYSA-N); Lomitapide (MBBCVAKAJPKAKM-UHFFFAOYSA-N); Lomondomycin (YDXARWIJAYOANV-FNORWQNLSA-N); Lomustine (GQYIWUVLTXOXAJ-UHFFFAOYSA-N); Lonafarnib (DHMTURDWPRKSOA-RUZDIDTESA-N); Lonaprisan (VHZPUDNSVGRVMB-RXDLHWJPSA-N); Lonidamine (WDRYRZXSPDWGEB-UHFFFAOYSA-N); Loperamide (RDOIQAHITMMDAJ-UHFFFAOYSA-N); Loperamide hydrochloride (PGYPOBZJRVSMDS-UHFFFAOYSA-N); Lopinavir (KJHKTHWMRKYKJE-SUGCFTRWSA-N); Loratadine (JCCNYMKQOSZNPW-UHFFFAOYSA-N); Lorcaserin (XTTZERNUQAFMOF-QMMMGPOBSA-N); Lorlatinib (IIXWYSCJSQVBQM-LLVKDONJSA-N); Lornoxicam (WLHQHAUOOXYABV-UHFFFAOYSA-N); Losmapimod (KKYABQBFGDZVNQ-UHFFFAOYSA-N); Lovastatin (PCZOHLXUXFIOCF-BXMDZJJMSA-N); Loxapine succinate (YQZBAXDVDZTKEQ-UHFFFAOYSA-N); Lucanthone (FBQPGGIHOFZRGH-UHFFFAOYSA-N); Lucanthone hydrochloride (LAOOXBLMIJHMFO-UHFFFAOYSA-N); Lucitanib (CUDVHEFYRIWYQD-UHFFFAOYSA-N); Luliconazole (YTAOBBFIOAEMLL-REQDGWNSSA-N); Lumacaftor (UFSKUSARDNFIRC-UHFFFAOYSA-N); Lumefantrine (DYLGFOYVTXJFJP-MYYYXRDXSA-N); Luminespib (NDAZATDQFDPQBD-UHFFFAOYSA-N); Lurbinectedin (YDDMIZRDDREKEP-HWTBNCOESA-N); Luteolin (IQPNAANSBPBGFQ-UHFFFAOYSA-N); Luteoskyrin (FAZDYVMEXQHRLI-UHFFFAOYSA-N); Luxeptinib (MWHHJYUHCZWSLS-UHFFFAOYSA-N); Lycopene (OAIJSZIZWZSQBC-GYZMGTAESA-N); Lymphosarcin (KBKJXVMKCWBZLB-UHFFFAOYSA-N); Lynestrenol (YNVGQYHLRCDXFQ-XGXHKTLJSA-N); Lysergide (VAYOSLLFUXYJDT-RDTXWAMCSA-N); M2698 free base (HXAUJHZZPCBFPN-QGZVFWFLSA-N); MBX-2982 (NFTMKHWBOINJGM-UHFFFAOYSA-N); MGCD-265 (UFICVEHDQUKCEA-UHFFFAOYSA-N); MIK665 (PKYIMGFMRFVOMB-LDLOPFEMSA-N); MK-0731 (MYBGWENAVMIGMM-GIFXNVAJSA-N); MK-0752 (XCGJIFAKUZNNOR-UHFFFAOYSA-N); MK-2206 (ULDXWLCXEDXJGE-UHFFFAOYSA-N); MK-2206 dihydrochloride (HWUHTJIKQZZBRA-UHFFFAOYSA-N); MK-2461 (JGEBLDKNWBUGRZ-HXUWFJFHSA-N); MK-5108 (LCVIRAZGMYMNNT-UHFFFAOYSA-N); MK-571 sodium (XNAYQOBPAXEYLI-AAGWESIMSA-M); MK-8033 (VMJFTOSOFDEKTM-UHFFFAOYSA-N); MK-8245 (UJEAABFSXKCSGI-UHFFFAOYSA-N); MK-8353 (KPQQGHGDBBJGFA-QNGWXLTQSA-N); MKC-1 (OVSKGTONMLKNPZ-UHFFFAOYSA-N); MKC3946 (IVQVBMWPWPTSNO-UHFFFAOYSA-N); MLN8054 (HHFBDROWDBDFBR-UHFFFAOYSA-N); MLS000591206 (CLEYUUPWDKQMML-UHFFFAOYSA-N); MLS000766263 (DPGPIFODEPGGKB-UHFFFAOYSA-N); MLS001202244 (PRGILOMAMBLWNG-UHFFFAOYSA-N); MLS002694120 (FVCSHCORWAOAEB-UHFFFAOYSA-N); MLS002695938 (BOJKULTULYSRAS-NXIQKPQYSA-N); MLS002701836 (YYDLFVZOIDOGSO-UHFFFAOYSA-N); MLS002701854 (QEHOIJJIZXRMAN-UHFFFAOYSA-N); MLS002702219 (ONFXWKXZNKTVMR-UHFFFAOYSA-N); MLS002702472 (NYRIXOYDRXFNGO-UHFFFAOYSA-N); MLS002702879 (OWPCHSCAPHNHAV-LLAMSIHESA-N); MLS002702909 (DWWALFYNQWNXFV-UHFFFAOYSA-N); MLS002702933 (SXQWVORVQPQIJG-UHFFFAOYSA-N); MLS002702959 (XNQPDXXSAUJASE-UHFFFAOYSA-N); MLS002703040 (VCEWFFSOGKSWLL-UHFFFAOYSA-N); MLS002920191 (JHZKUIUDOKIAQC-UHFFFAOYSA-N); MLS003106944 (IYJACKLHQZCAOV-UMDHDWCXSA-N); MLS003115198 (ZBBKGJCZNKXCAO-UHFFFAOYSA-N); MLS003115218 (IXIUIXFHMVRZSY-UHFFFAOYSA-N); MLS003115680 (MMEBJLATRATDOX-LDADJPATSA-N); MLS003170673 (MZACWVULBYILQW-SFQUDFHCSA-N); MTX-211 (GUYKCXXNIKKSBC-UHFFFAOYSA-N); MX2 HCl (ZDJRSUWWMAYYID-HATHGVPASA-N); Macbecin ii (SVSFCSOFEPJFSF-FOKZELFKSA-N); Macrophilone a (LUJDHAGJNKEFEN-UHFFFAOYSA-N); Mafenide (TYMRLRRVMHJFTF-UHFFFAOYSA-N); Malachite green oxalate (CNYGFPPAGUCRIC-UHFFFAOYSA-L); Malonoben (MZOPWQKISXCCTP-UHFFFAOYSA-N); Malononitrile, (m-nitrocinnamylidene)-(SUUDBEARADVQJK-HNQUOIGGSA-N); Malotilate (YPIQVCUJEKAZCP-UHFFFAOYSA-N); Manganous chloride (GLFNIEUTAYBVOC-UHFFFAOYSA-L); Manidipine (ANEBWFXPVPTEET-UHFFFAOYSA-N); Manidipine dihydrochloride (JINNGBXKBDUGQT-UHFFFAOYSA-N); Maprotiline (QSLMDECMDJKHMQ-UHFFFAOYSA-N); Maprotiline hydrochloride (NZDMFGKECODQRY-UHFFFAOYSA-N); Maraviroc (GSNHKUDZZFZSJB-HLMSNRGBSA-N); Mardepodect (AZEXWHKOMMASPA-UHFFFAOYSA-N); Marimastat (OCSMOTCMPXTDND-OUAUKWLOSA-N); Marinopyrrole a (QYPJBTMRYKRTFG-UHFFFAOYSA-N); Marizomib (NGWSFRIPKNWYAO-SHTIJGAHSA-N); Maropitant (OMPCVMLFFSQFIX-CONSDPRKSA-N); Masitinib (WJEOLQLKVOPQFV-UHFFFAOYSA-N); Masoprocol (HCZKYJDFEPMADG-TXEJJXNPSA-N); Mauve ion (GZIROVCEVALULC-UHFFFAOYSA-O) salts; Mavacamten (RLCLASQCAPXVLM-NSHDSACASA-N); Mavelertinib (JYIUNVOCEFIUIU-GHMZBOCLSA-N); Maytansin (WKPWGQKGSOKKOO-WLNOZUDTSA-N); Mayzent (KIHYPELVXPAIDH-APTWKGOFSA-N); Mebendazole (OPXLLQIJSORQAM-UHFFFAOYSA-N); Mebeverine hydrochloride (PLGQWYOULXPJRE-UHFFFAOYSA-N); Mebhydrolin napadisilate (CJUOSBUQOWKEKJ-UHFFFAOYSA-N); Mechlorethamine (HAWPXGHAZFHHAD-UHFFFAOYSA-N); Mechlorethamine hydrochloride (QZIQJVCYUQZDIR-UHFFFAOYSA-N); Meclinertant (DYLJVOXRWLXDIG-UHFFFAOYSA-N); Meclizine (OCJYIGYOJCODJL-UHFFFAOYSA-N); Meclizine hydrochloride (VCTHNOIYJIXQLV-UHFFFAOYSA-N); Meclocycline sulfosalicylate (WVJKUGVVYXCLFV-CCHMMTNSSA-N); Meclofenamate sodium (OGPIIGMUPMPMNT-UHFFFAOYSA-M); Meclofenamic acid (SBDNJUWAMKYJOX-UHFFFAOYSA-N); Medazepam (YLCXGBZIZBEVPZ-UHFFFAOYSA-N); Medroxyprogesterone (FRQMUZJSZHZSGN-HBNHAYAOSA-N); Medroxyprogesterone acetate (PSGAAPLEWMOORI-PEINSRQWSA-N); Medrysone (GZENKSODFLBBHQ-ILSZZQPISA-N); Mefloquine (XEEQGYMUWCZPDN-UHFFFAOYSA-N); Mefloquine hydrochloride (WESWYMRNZNDGBX-YLCXCWDSSA-N); Megestrol acetate (RQZAXGRLVPAYTJ-GQFGMJRRSA-N); Melatonin (DRLFMBDRBRZALE-UHFFFAOYSA-N); Melitracen hydrochloride (RADLXCPDUXFGFF-UHFFFAOYSA-N); Melperone (DKMFBWQBDIGMHM-UHFFFAOYSA-N); Melphalan (SGDBTWWWUNNDEQ-LBPRGKRZSA-N); Melphalan hydrochloride (OUUYBRCCFUEMLH-YDALLXLXSA-N); Memantine (BUGYDGFZZOZRHP-UHFFFAOYSA-N); Menadione (MJVAVZPDRWSRRC-UHFFFAOYSA-N); Menadione sodium bisulfite (XDPFHGWVCTXHDX-UHFFFAOYSA-M); Menatetrenone (DKHGMERMDICWDU-GHDNBGIDSA-N); Menthyl anthranilate (SOXAGEOHPCXXIO-UHFFFAOYSA-N); Mepazine acetate (COBIOCPXKOZHSU-UHFFFAOYSA-N); Mephaquin; Racemic mefloquine; Ro 21-5998; WR 142490 (WESWYMRNZNDGBX-UHFFFAOYSA-N); Meprednisone (PIDANAQULIKBQS-RNUIGHNZSA-N); Mepyramine (YECBIJXISLIIDS-UHFFFAOYSA-N); Mequitazine (HOKDBMAJZXIPGC-UHFFFAOYSA-N); Mercaptopurine (GLVAUDGFNGKCSF-UHFFFAOYSA-N); Mercury, [mu-[orthoborato(2-)-O:O′]]diphenyldi-(HCIUVGKUNYOVBM-UHFFFAOYSA-N); Mercury, chloro-2-naphthalenyl-(JEWKITRCRXNBDK-UHFFFAOYSA-M); Merestinib (QHADVLVFMKEIIP-UHFFFAOYSA-N); Merimepodib (JBPUGFODGPKTDW-SFHVURJKSA-N); Mesalamine (KBOPZPXVLCULAV-UHFFFAOYSA-N); Mesna (XOGTZOOQQBDUSI-UHFFFAOYSA-M); Mesoridazine (SLVMESMUVMCQIY-UHFFFAOYSA-N); Mestranol (IMSSROKUHAOUJS-MJCUULBUSA-N); Met kinase inhibitor (FPYJSJDOHRDAMT-KQWNVCNZSA-N); Metarrestin (WSMXAUJFLWRPNT-UHFFFAOYSA-N); Metergoline (WZHJKEUHNJHDLS-QTGUNEKASA-N); Metformin hydrochloride (OETHQSJEHLVLGH-UHFFFAOYSA-N); Methacycline hydrochloride (VXPSARQTYDZXAO-CCHMMTNSSA-N); Methadone hydrochloride (FJQXCDYVZAHXNS-UHFFFAOYSA-N); Methanesulfon-m-anisidide, monomethanesulfonate (ZMEYIYCLMKYQNO-UHFFFAOYSA-N); Methanone, (4-methoxyphenyl)(3,4,5-trimethoxyphenyl)-(GGZQGMKOFKNKQN-UHFFFAOYSA-N); Methapyrilene hydrochloride (BONORRGKLJBGRV-UHFFFAOYSA-N); Methaqualone (JEYCTXHKTXCGPB-UHFFFAOYSA-N); Methasquin (NTKNUVXCGJPJQP-UHFFFAOYSA-N); Methazolamide (FLOSMHQXBMRNHR-UHFFFAOYSA-N); Methdilazine hydrochloride (IEISBKIVLDXSMZ-UHFFFAOYSA-N); Methixene hydrochloride (RAOHHYUBMJLHNC-UHFFFAOYSA-N); Methotrexate (FBOZXECLQNJBKD-ZDUSSCGKSA-N); Methoxsalen (QXKHYNVANLEOEG-UHFFFAOYSA-N); Methyl 3-[(1-nitroacridin-9-yl)amino]propanoate (LXKKFMDQYDSSEW-UHFFFAOYSA-N); Methyl 5,6,11-trimethylpyrido[4,3-b]carbazole-9-carboxylate (QABNHIUOPCPVOU-UHFFFAOYSA-N); Methyl ester prodigiosene (LJLCDJJUJPXYMF-ZTXYIFKNSA-N); Methyl linolenate (DVWSXZIHSUZZKJ-YSTUJMKBSA-N); Methyl methanesulfonate (MBABOKRGFJTBAE-UHFFFAOYSA-N); Methyl-Gag (RRECQJYPJNWFNK-BCKSSGNJSA-N); Methyl-azatoxin (SCLLIEKCYQJGGW-SZQRVLIRSA-N); Methyldopa (CJCSPKMFHVPWAR-JTQLQIEISA-N); Methyldopate hydrochloride (QSRVZCCJDKYRRF-YDALLXLXSA-N); Methylene blue (CXKWCBBOMKCUKX-UHFFFAOYSA-M); Methylene blue cation ion (RBTBFTRPCNLSDE-UHFFFAOYSA-N) salts; Methylene blue trihydrate (XQAXGZLFSSPBMK-UHFFFAOYSA-M); Methylprednisolone (VHRSUDSXCMQTMA-PJHHCJLFSA-N); Methyltestosterone (GCKMFJBGXUYNAG-HLXURNFRSA-N); Methyltrioctylammonium bis(trifluoromethylsulfonyl)imide (LAGQNGWYNLUQRI-UHFFFAOYSA-N); Methysergide (KPJZHOPZRAFDTN-ZRGWGRIASA-N); Methysergide maleate (LWYXFDXUMVEZKS-ZVFOLQIPSA-N); Metildigoxin (IYJMSDVSVHDVGT-PEQKVOOWSA-N); Metixene hydrochloride (RSXZRFHNNTTWCB-UHFFFAOYSA-N); Metolazone (AQCHWTWZEMGIFD-UHFFFAOYSA-N); Metoserpate hydrochloride (RVUCXCLULXWKIQ-PKNINNMNSA-N); Metronidazole (VAOCPAMSLUNLGC-UHFFFAOYSA-N); Metyrapone (FJLBFSROUSIWMA-UHFFFAOYSA-N); Metyrosine (NHTGHBARYWONDQ-JTQLQIEISA-N); Mevastatin (AJLFOPYRIVGYMJ-INTXDZFKSA-N); Mianserin (UEQUQVLFIPOEMF-UHFFFAOYSA-N); Mibampator (ULRDYYKSPCRXAJ-KRWDZBQOSA-N); Mibefradil dihydrochloride (MTJLQTFHJIHXIX-GDUXWEAWSA-N); Miconazole (BYBLEWFAAKGYCD-UHFFFAOYSA-N); Miconazole nitrate (MCCACAIVAXEFAL-UHFFFAOYSA-N); Midodrine (PTKSEFOSCHHMPD-UHFFFAOYSA-N); Midostaurin (BMGQWWVMWDBQGC-IIFHNQTCSA-N); Mifepristone (VKHAHZOOUSRJNA-GCNJZUOMSA-N); Milademetan (RYAYYVTWKAOAJF-QISPRATLSA-N); Milciclib (RXZMYLDMFYNEIM-UHFFFAOYSA-N); Milrinone (PZRHRDRVRGEVNW-UHFFFAOYSA-N); Miltefosine (PQLXHQMOHUQAKB-UHFFFAOYSA-N); Minaprine dihydrochloride (GNUCGROXDZMCJI-UHFFFAOYSA-N); Minnelide (ZHBJMVNZRZUQEP-KIKMAQITSA-L); Minocycline hydrochloride (WTJXVDPDEQKTCV-VQAITOIOSA-N); Minoxidil (ZIMGGGWCDYVHOY-UHFFFAOYSA-N); Miransertib (HNFMVVHMKGFCMB-UHFFFAOYSA-N); Miransertib hcl (DRHSWSSVIKDJME-UHFFFAOYSA-N); Misonidazole (OBBCSXFCDPPXOL-UHFFFAOYSA-N); Mitindomide (DRCJGCOYHLTVNR-UHFFFAOYSA-N); Mito-tempol (AIQMYWZQFQQTIQ-UHFFFAOYSA-M); Mitoflaxone (TZZNWMJZDWYJAZ-UHFFFAOYSA-N); Mitoguazone (MXWHMTNPTTVWDM-NXOFHUPFSA-N); Mitomycin a (HYFMSAFINFJTFH-NGSRAFSJSA-N); Mitomycin c (NWIBSHFKIJFRCO-WUDYKRTCSA-N); Mitotane (JWBOIMRXGHLCPP-UHFFFAOYSA-N); Mitoxantrone (KKZJGLLVHKMTCM-UHFFFAOYSA-N); Mitoxantrone hydrochloride (ZAHQPTJLOCWVPG-UHFFFAOYSA-N); Mitozolomide (QXYYYPFGTSJXNS-UHFFFAOYSA-N); Mitramycin (CFCUWKMKBJTWLW-OQCYXQHVSA-N); Miuraenamide a (LOOHMHDNJMUEAT-SFHJFPFYSA-N); Mivavotinib (MJHOMTRKVMKCNE-NWDGAFQWSA-N); Mivebresib (RDONXGFGWSSFMY-UHFFFAOYSA-N); Mivobulin isethionate (JEFPWOBULVSOTM-PPHPATTJSA-N); Mizolastine (PVLJETXTTWAYEW-UHFFFAOYSA-N); Mkt 077 (VSKYOTRJSLYFHX-UXJRWBAGSA-M); Mobocertinib (AZSRSNUQCUDCGG-UHFFFAOYSA-N); Mocetinostat (HRNLUBSXIHFDHP-UHFFFAOYSA-N); Mocravimod (IINUNQPYJGJCJI-UHFFFAOYSA-N); Molidustat (IJMBOKOTALXLKS-UHFFFAOYSA-N); Molindone hydrochloride (GQWNECFJGBQMBO-UHFFFAOYSA-N); Momelotinib (ZVHNDZWQTBEVRY-UHFFFAOYSA-N); Mometasone furoate (WOFMFGQZHJDGCX-ZULDAHANSA-N); Monoacetyl verrucarin a epoxide (JDFKYDYICWYZMF-KQQUZDAGSA-N); Monoanhydride with sulfuric acid (KRPNDEFBHRAWPW-UHFFFAOYSA-M); Monobenzone (VYQNWZOUAUKGHI-UHFFFAOYSA-N); Monomethyl auristatin e (DASWEROEPLKSEI-UIJRFTGLSA-N); Montelukast sodium (LBFBRXGCXUHRJY-HKHDRNBDSA-M); Morin (YXOLAZRVSSWPPT-UHFFFAOYSA-N); Morphine (BQJCRHHNABKAKU-KBQPJGBKSA-N); Morphine sulfate (GRVOTVYEFDAHCL-RTSZDRIGSA-N); Morpholinoadriamycin (INAUWOVKEZHHDM-PYBHCKQVSA-N); Mosapride (YPELFRMCRYSPKZ-UHFFFAOYSA-N); Mosapride citrate (HUZTYZBFZKRPFG-UHFFFAOYSA-N); Motesanib (RAHBGWKEPAQNFF-UHFFFAOYSA-N); Motesanib diphosphate (ONDPWWDPQDCQNJ-UHFFFAOYSA-N); Motolimod (QSPOQCXMGPDIHI-UHFFFAOYSA-N); Moxaverine (MYCMTMIGRXJNSO-UHFFFAOYSA-N); Moxisylyte (VRYMTAVOXVTQEF-UHFFFAOYSA-N); Moxonidine (WPNJAUFVNXKLIM-UHFFFAOYSA-N); Mozavaptan (WRNXUQJJCIZICJ-UHFFFAOYSA-N); Mubritinib (ZTFBIUXIQYRUNT-MDWZMJQESA-N); Mycophenolate mofetil (RTGDFNSFWBGLEC-SYZQJQIISA-N); Mycophenolic acid (HPNSFSBZBAHARI-RUDMXATFSA-N); N′-((6-Methyl-2-pyridinyl)methylene)-1-pyrrolidinecarbothiohydrazide (NTPQBGPSNWNHFH-UKTHLTGXSA-N); N′-(1-(Pyridin-2-yl)ethylidene)azetidine-1-carbothiohydrazide (XDHBUMNIQRLHGO-UKTHLTGXSA-N); N′-(2-Pyridinylmethylene)-1-piperidinecarbothiohydrazide (SLOCYEFBRCHUHA-GXDHUFHOSA-N); N′-(3-pyridin-2-ylisoquinolin-1-yl)pyridine-2-carboximidamide (VZJMIYLTPATHJF-UHFFFAOYSA-N); N, O-Diethoxyacetyl-3-demethyldeactylthiocolchicin (NGSBHZFTKFWKBZ-IBGZPJMESA-N); N,N′-Bis(9-acridinyl)-1,6-hexanediamine (FZSBSJDISGQMLW-UHFFFAOYSA-N); N,N′-Diphenyl-2-(tosylamino)pentanediamide (XLALRUIHDDSPFR-UHFFFAOYSA-N); N,N′-bis(1-nitroacridin-9-yl)pentane-1,5-diamine (NNOSNVDRJWZODO-UHFFFAOYSA-N); N,N′-bis(4-iodophenyl)[1,2,5]oxadiazolo[3,4-b]pyrazine-5,6-diamine (KXBGCIMRYZMVCD-UHFFFAOYSA-N); N,N,N-Trimethyl(2,3,4,6-tetramethylphenyl)methanaminium chloride (FKOBRGCOCFTSPZ-UHFFFAOYSA-M); N,N-Bis(2-chloroethyl)aniline (ROSJKFFLIXTTAW-UHFFFAOYSA-N); N,N-Dibutyl-7-(diethylamino)-9-methyl-3H-phenothiazin-3-iminium chloride (RMODMLJGOYRGMV-UHFFFAOYSA-M); N,N-Dimethyldaunomycin hydrochloride (LXUULYQZJJTFOD-MUUKTGSOSA-N); N,N-dibutyl-4-[4-(dibutylamino)benzenecarboximidoyl]aniline; hydrochloride (UFNSXKNAQJUKKK-UHFFFAOYSA-N); N-(1-Methyl-5-indolyl)-N′-(3-pyridyl)urea (OJZZJTLBYXHUSJ-UHFFFAOYSA-N); N-(1-Methylethyl)-N′-(1-nitro-9-acridinyl)-1,3-propanediamine (QXNCBKQFKXJKGD-UHFFFAOYSA-N); N-(2,4,6-trinitrophenyl)carbazol-9-amine (FRLJBDXFYGRMJG-UHFFFAOYSA-N); N-(2,6-Dichlorobenzylidene)-N′-amidinohydrazine (WDZVGELJXXEGPV-YIXHJXPBSA-N); N-(3,5-bis(trifluoromethyl)phenyl)-5-chloro-2-hydroxybenzamide (CHILCFMQWMQVAL-UHFFFAOYSA-N); N-(3-bromophenyl)-6,7-dimethoxyquinazolin-4-amine (LSPANGZZENHZNJ-UHFFFAOYSA-N); N-(3-chlorophenyl)-6,7-dimethoxyquinazolin-4-amine (GFNNBHLJANVSQV-UHFFFAOYSA-N); N-(3-cyano-4-methyl-1H-indol-7-yl)-3-cyanobenzene-sulfonamide (LWGUASZLXHYWIV-UHFFFAOYSA-N); N-(3-fluorophenyl)-6,7-dimethoxy-1,4-dihydroindeno[1,2-c]pyrazol-3-amine (ZDNURMVOKAERHZ-UHFFFAOYSA-N); N-(3-methylphenyl)-4-pyridin-4-yl-1,3-thiazol-2-amine (KATNUHQNJGNLPW-UHFFFAOYSA-N); N-(4-((3-bromophenyl)amino)quinazolin-6-yl)but-2-ynamide (BTYYWOYVBXILOJ-UHFFFAOYSA-N); N-(4-(4-Methyl-1-piperazinyl)phenyl)-9-acridinamine (QZKGUNQLVFEEBA-UHFFFAOYSA-N); N-(4-(5-Bromo-2-pyrimidinyloxy)-3-chlorophenyl)-N′-(2-nitrobenzoyl)urea (WKXWMGOTZJGIIK-UHFFFAOYSA-N); N-(4-Methoxybenzyl)-N′-(5-nitro-1,3-thiazol-2-YL)urea (YAEMHJKFIIIULI-UHFFFAOYSA-N); N-(4-methoxyphenyl)-N,5-dimethylfuro[2,3-d]pyrimidin-4-amine (UVZUIESPIIIPQW-UHFFFAOYSA-N); N-(5-benzyl-1,3-thiazol-2-yl)-2,3-dihydro-1,4-benzodioxine-6-carboxamide (WQPYYFYIRJODTF-UHFFFAOYSA-N); N-(5-methoxynaphthalen-2-yl)-N,2-dimethylthieno[3,2-d]pyrimidin-4-amine (MTHGUBBFWBERGE-UHFFFAOYSA-N); N-(5-methoxynaphthalen-2-yl)-N-methylquinazolin-4-amine (HELYUAQHXHRESA-UHFFFAOYSA-N); N-(6-chloro-9 h-pyrido[3,4-b]indol-8-yl)-3-pyridinecarboxamide (JZRMBDHPALEPDM-UHFFFAOYSA-N); N-(Phenoxyacetyl)deacetylcolchicine (GQILPBLGDOWLSP-UHFFFAOYSA-N); N-(Vinyloxycarbonyl)deacetylthiocolchicine (QNPQOSKMAOKAKA-INIZCTEOSA-N); N-(dipyridin-2-ylmethylideneamino)-1,3-benzoxazol-2-amine (KSUHGVVTBOMMPD-UHFFFAOYSA-N); N-Benzoyldeacetylthiocolchicine (AHNUEGAJJOXHHL-UHFFFAOYSA-N); N-Butyryldeacetylcolchicine (XWHJWSRKXWWZRN-UHFFFAOYSA-N); N-Deacetylthiocolchicine (NUNCOHUMTCDISK-AWEZNQCLSA-N); N-Hydroxy-2-((1-phenylcyclopropyl)amino)pyrimidine-5-carboxamide (LIIWIMDSZVNYHY-UHFFFAOYSA-N); N-Isopropyl-N′-phenyl-p-phenylenediamine (OUBMGJOQLXMSNT-UHFFFAOYSA-N); N-Nitroso-N-methylurea (ZRKWMRDKSOPRRS-UHFFFAOYSA-N); N-Trifluoroacetyladriamycin-14-valerate (ZOCKGBMQLCSHFP-ZQUOIQDWSA-N); N-[(E)-1-pyridin-2-ylethylideneamino]piperidine-1-carbothioamide (JLQJXIRORURHRB-RVDMUPIBSA-N); N-[(E)-1-pyridin-2-ylethylideneamino]pyrrolidine-1-carbothioamide (KJWUMRFUJFIVGM-GXDHUFHOSA-N); N-[(E)-[phenyl(pyridin-2-yl)methylidene]amino]pyridin-2-amine; zinc; acetate ion (LKQQGBLWMYDLBX-PJPLBZAPSA-M) salts; N-[(Z)-(6-methylpyridin-2-yl)methylideneamino]piperidine-1-carbothioamide (YFCZEYSSYGJTIZ-UVTDQMKNSA-N); N-[(Z)-[phenyl(pyridin-2-yl)methylidene]amino]azepane-1-carbothioamide (RMVYAGRPWKVQOQ-UZYVYHOESA-N); N-[(Z)-[phenyl(pyridin-2-yl)methylidene]amino]piperidine-1-carbothioamide (WNCDDLCNZKQXCV-JZJYNLBNSA-N); N-[1-(6-methyl-2-pyridinyl)ethylideneamino]-1-piperidinecarbothioamide (PVHGAKSBSCSRFX-FOWTUZBSSA-N); N-[1-[(6-chloro-3-pyridinyl)methyl]-4,5-dihydroimidazol-2-yl]nitramide (YWTYJOPNNQFBPC-UHFFFAOYSA-N); N-[2-(dimethylamino)ethyl]-6-nitronaphthalene-1-carboxamide; hydrochloride (OHGOSYFAOBJNLU-UHFFFAOYSA-N); N-[3-(4-benzamidopyrrolo[3,2-c]pyridin-1-yl)phenyl]-3,4-dichlorobenzamide (RDRHRIYSWMLPJS-UHFFFAOYSA-N); N-[4-[(3-aminoacridin-9-yl)amino]phenyl]methanesulfonamide; hydrochloride (OWGULQJEIFFGLM-UHFFFAOYSA-N); N-[4-[(3-methoxyacridin-9-yl)amino]phenyl]methanesulfonamide (IHPUDZSNMQRJEV-UHFFFAOYSA-N); N-[9-(2-phenylethylamino)acridin-3-yl]-3-pyrrolidin-1-ylpropanamide (BUZVZVXFUHTTGC-UHFFFAOYSA-N); N-[9-(benzylamino)acridin-3-yl]-3-pyrrolidin-1-ylpropanamide (ROSMYOIOEDKWQH-UHFFFAOYSA-N); N-[9-[4-(dimethylamino)anilino]acridin-3-yl]-3-pyrrolidin-1-ylpropanamide (HDAIKUIUEOSAFC-UHFFFAOYSA-N); N-[p-[[(2,4-Diamino-6-quinazolinyl)methyl]amino]benzoyl]-L-aspartic acid (RJMVWPJFVJJJLO-UHFFFAOYSA-N); N-benzyl-2-(5-(4-(2-morpholinoethoxy)phenyl)pyridin-2-yl)acetamide (HUNGUWOZPQBXGX-UHFFFAOYSA-N); N-ethyl-N-(2-methylphenyl)but-2-enamide (DNTGGZPQPQTDQF-UHFFFAOYSA-N); N-hydroxy-1-(4-methoxybenzyl)-1H-indole-6-carboxamide (AJRGHIGYPXNABY-UHFFFAOYSA-N); N-hydroxy-7-(9-methoxy-6,11-dioxoindeno[1,2-c]quinolin-5-yl)heptanamide (SZXGEFJTMRNBAN-UHFFFAOYSA-N); N-methyl-6-pyrrolidin-1-yl-2-[3-(trifluoromethoxy)phenyl]quinolin-4-amine (WPWUOJWDZBZUCT-UHFFFAOYSA-N); N-p-Chlorobenzyl-N′,N′-dimethyl-N-2-pyridylethylenediamine hydrochloride (VEYWWAGBHABATA-UHFFFAOYSA-N); N1,2-diamine (HQVFBVTYAIBOSR-UHFFFAOYSA-N); N6-Cyclohexyl-N2-(2-methyl-4-morpholinophenyl)-9H-purine-2,6-diamine (OVJBNYKNHXJGSA-UHFFFAOYSA-N); NAPHTHO(2,1-b)FURAN-1-ETHANOL, 2-NITRO-(DMTZMRKTFPMXSU-UHFFFAOYSA-N); NCGC00094807-01 (BJSPSQYEAXWPOK-UHFFFAOYSA-N); NCGC00095992-01 (IUAYMJGZBVDSGL-UHFFFAOYSA-N); NCI60_041054 (CBSVWOFEDIRIAW-UHFFFAOYSA-N); NFATc1-IN-1 (IGRCFOGPIJEESA-UHFFFAOYSA-N); NMS-E973 (YLQODGGPIHWTHR-UHFFFAOYSA-N); NMS-P118 (ARYVAQSYRLZVQD-UHFFFAOYSA-N); NSC 26480 (5,6-dihydro-5-azacytidine) (MRFRLYAOBSTHHU-UHFFFAOYSA-N); NSC 330770 (RLHKYVHZNZMLRH-UHFFFAOYSA-N); NSC 364573 (formerly) (HHBSFXFIMWLBAI-JOQJRFOHSA-N); NSC 632839 hydrochloride (ZOKZLTXPTLIWOJ-BYCVLTJGSA-N); NSC 655649; XL 119; bms 181176; bmy 27557 (JSKFWUPVIZYJMR-UHFFFAOYSA-N); NSC-279503 (NTBOLWMPXFGUHO-UHFFFAOYSA-N); NSC-639366 (BREXYFHGCPUYSM-WLHGVMLRSA-N); NSC-654663 (DZMVCVHATYROOS-CKXIHPEFSA-N); NSC-724448 (PMZOKGKZIAJGIK-KLCVKJMQSA-N); NSC-748393 (VWMMYRCUWSIIBR-UHFFFAOYSA-N); NSC-748394 (KKTYQIZMCZOJPP-UHFFFAOYSA-N); NSC-751382 (NNVDIXHOVATNMH-UHFFFAOYSA-N); NSC102866 (FIVPIPIDMRVLAY-UHFFFAOYSA-N); NSC10594 (JBANJYHYPHFVFW-UHFFFAOYSA-N); NSC109682 (QMXPFFBRRUJWPN-UHFFFAOYSA-M); NSC113089 ion (JAVKJNWMCBCERK-UHFFFAOYSA-O) salts; NSC114347 ion (BSSFQMTYHWRXDZ-UHFFFAOYSA-O) salts; NSC126728 (UYMAFCXKKGDQQU-VBRYSZMJSA-N); NSC128687 (MHEIPMMGYDKOAW-UHFFFAOYSA-N); NSC134033 (CEUFDEOPWBBLCY-UHFFFAOYSA-N); NSC136044 (NWPIUETWDSWOKV-QROLFQDOSA-N); NSC14657 ion (VUTWKJOBQDFUAW-UHFFFAOYSA-M) salts; NSC174121 (XNZUKUJONMYGIN-UHFFFAOYSA-N); NSC184692 (MALRUKQRDHTJPO-UHFFFAOYSA-N); NSC200736 (UYMAFCXKKGDQQU-CAOZSPNBSA-N); NSC238181 (WAYNIXHIORQRDI-VMWQHYTCSA-N); NSC254681 (KVTVLWOKIDKIQL-LAFLKOLFSA-N); NSC256439 (XDXDZDZNSLXDNA-UHFFFAOYSA-N); NSC260642 (GEKAQVYZDJWDOO-UHFFFAOYSA-N); NSC26111 (HDNRJBPQWIJEIW-UHFFFAOYSA-N); NSC26113 (RKERNVBCOIOPFR-QPJJXVBHSA-N); NSC266210 (ACPOUBCJPBQMFD-BCNBNTECSA-N); NSC271967 (GWPCKEZKGMFTSM-UHFFFAOYSA-M); NSC273432 (WWVGIBNAHCRGSA-JCAINFKWSA-N); NSC27413 (JPMMSGWPIKKAFL-UHFFFAOYSA-N); NSC281617 (PCMRUMCRHVAFDB-UHFFFAOYSA-N); NSC28566 (DKFBZYCLVJIMOT-UHFFFAOYSA-N); NSC299933 (WILGDJPLXKHUEY-UDWIEESQSA-N); NSC301460 (DAWXCYYFNBEVJN-UHFFFAOYSA-N); NSC305787 (hydrochloride) (JQALIVSYOXLOBE-UHFFFAOYSA-N); NSC323241 (QZCGQJFNGZOSQK-UHIRCJBMSA-N); NSC332890 (LLOSSPFQKMGMHB-UHFFFAOYSA-N); NSC33410 (XTSSXTWGEJTWBM-FQEVSTJZSA-N); NSC335791 (SXEPBWUAUNIYCG-LVZFUZTISA-N); NSC335794 (TYTDQQOGLGLLNX-OQKWZONESA-N); NSC338005 (TVPBJULSXPLFIN-JQIJEIRASA-N); NSC339866 (BCRRQTMYMQMOAZ-UHFFFAOYSA-N); NSC343231 (HCIKQMMYJWBWCY-UHFFFAOYSA-M); NSC34927 ion (JBANJYHYPHFVFW-UHFFFAOYSA-M) salts; NSC352890 (ABQJRAXGQPSYFZ-UHFFFAOYSA-N); NSC354670 (OHHANFDQXBLKIW-MHWRWJLKSA-N); NSC355637 (NLHICQCHEWUHCR-UHFFFAOYSA-N); NSC361792 (PKYOHQGXPPVIGD-UHFFFAOYSA-N); NSC364372 (YACHGFWEQXFSBS-SAYXANOCSA-N); NSC368671 (RULBXOABLNFKRW-OUKQBFOZSA-N); NSC370589 (QTVNIFNYIYCKMI-VCHYOVAHSA-N); NSC372486 (BJAJYTAHDSJTTM-SFHVURJKSA-N); NSC374998 (JLEOLHIYYWXFTJ-DEDYPNTBSA-N); NSC376266 (OGHLKBYOYCGJPR-GHRIWEEISA-N); NSC37888 (UTSWYDVSMNDGNN-UHFFFAOYSA-N); NSC37896 (USFAHBCJIKVFCT-UHFFFAOYSA-N); NSC37898 (BGGJJODVQUHRLH-UHFFFAOYSA-N); NSC403163 (SDUGGZXPGJTZJV-UHFFFAOYSA-M); NSC529861 (GWIBVUFMBFNYLT-UHFFFAOYSA-N); NSC5920 (BTDQKVAGLWNJLK-UHFFFAOYSA-N); NSC625538 (LYLWYSBJLQWRSM-XQKVXRCISA-L); NSC628676 (UCWYAMAHWNCEOU-FEKGNFRDSA-N); NSC631197 (IUYGRRZDUIAIQJ-UHFFFAOYSA-N); NSC639655 (OYXKDQMOCVNLBP-UHFFFAOYSA-N); NSC639659 (GYCDKMVFGHDCQH-UHFFFAOYSA-N); NSC639831 (XVSHGAAPBNVZEJ-YTTHPEARSA-N); NSC639832 (XFXSVFLGSJQVAJ-RTGPMSLNSA-N); NSC642328 (WGEAIKSPJFUISL-WXIMRCAWSA-N); NSC643248 (ORVYOCOKBVMPLA-XGBKAKJLSA-N); NSC646395 (FLKXDNDCOXXZNL-UHFFFAOYSA-N); NSC646846 (PFRKTXMXGDERHT-BFTYWVKESA-N); NSC648774 (KALNLOJBUCRQOO-UHFFFAOYSA-N); NSC649890 (LGMSNQNWOCSPIK-KELGLJHESA-N); NSC650426 (LQIPDFIUPOYMPR-AQASXUMVSA-N); NSC650471 (OWPWFVVPBYFKBG-AMJZKRQHSA-N); NSC651079 (NPSQJYCWTMTMAH-UHFFFAOYSA-N); NSC651086 (DQBAFHSEZZMUNI-UHFFFAOYSA-N); NSC651849 (ZDPBIYQIWKMCQV-HIKCRJBTSA-N); NSC652886 (MEVZJLUKTABENS-UHFFFAOYSA-N); NSC652888 (MJMCRKZYGYOTHI-UHFFFAOYSA-N); NSC664976 (IOTWKLDMBZIDBV-UHFFFAOYSA-N); NSC665804 (SDFIROAOIOUHKX-BRNRUIPFSA-N); NSC666735 (IRKSSGDJSSMPGG-UHFFFAOYSA-N); NSC667645 (FUESXAKVEGFKGL-UHFFFAOYSA-N); NSC667885 (SOFPDFXZPLBDHW-RQZCQDPDSA-N); NSC668404 (JKNUSPYZNOLBAF-MHHARFCSSA-N); NSC670228 (IGOOXIYIMTXTPM-WLHGVMLRSA-N); NSC670701 (DYVCHPFZWVWSDD-UHFFFAOYSA-N); NSC670702 (WNYBRSMBKFFCBV-UHFFFAOYSA-N); NSC670968 (VXLQGSTWRCRJRA-LDADJPATSA-N); NSC671031 (MBHBKYUJERKJAW-UHFFFAOYSA-N); NSC671136 (MSLAPUVHAQOMIR-UHFFFAOYSA-N); NSC67734 (YIJDNFUDTIIWOG-UHFFFAOYSA-N); NSC677599 (FCLWHDIQAVOPMW-UHFFFAOYSA-N); NSC681228 (RCTFPTUYPPNJBH-ZCYQVOJMSA-N); NSC681229 (XNHKOPOMEWQJJQ-QJHJCNPRSA-N); NSC681237 (WAMUQAMOLUMIBL-APTPAJQOSA-N); NSC683414 (JKDAETVNFABJQD-GEYQZXRLSA-N); NSC683415 (WEDSSFYVXYLQLK-DVMCIEKCSA-N); NSC691039 (JHFPQOLAHRTDAC-AWEZNQCLSA-N); NSC692738 (IMDGOWFSIBTXGN-UHFFFAOYSA-N); NSC693546 (DRTNXALIYSNZIV-UHFFFAOYSA-N); NSC697537 (ARHBFRMLLOEANX-UHFFFAOYSA-N); NSC698104 (VRBJCJNVLXEKKP-MDZDMXLPSA-N); NSC698932 (NRHDGIYFJJUFKN-AWEZNQCLSA-N); NSC700013 (XJQGAAFLJAOXBC-DHZHZOJOSA-N); NSC700912 (VOKPQOKKNBHZIX-INIZCTEOSA-N); NSC701747 (CWQIJVNYJRUBQW-FPLPWBNLSA-N); NSC710551 (CSPZQMQCDUYWIE-UHFFFAOYSA-N); NSC710552 (SMROYVOGMSQMJK-UHFFFAOYSA-N); NSC710556 (FRNLBJJQLDHPJL-UHFFFAOYSA-N); NSC710559 (TYSQOYAQKRRQCQ-UHFFFAOYSA-N); NSC734562 (NXGGPXFLOUDJQA-UHFFFAOYSA-N); NSC741107 (ORWUSSJLUPFYCH-UHFFFAOYSA-N); NSC741299 (JVDJXOLMFJIQMD-FOKLQQMPSA-N); NSC743121 (ZOMBISVGMSQISI-UHFFFAOYSA-N); NSC743122 (ZLTJRBWBVYPAME-UHFFFAOYSA-N); NSC743134 (OVOQKQFNIMCAPH-UHFFFAOYSA-N); NSC743135 (QDKRGBUDNUCBGJ-UHFFFAOYSA-N); NSC743433 (BOCPXUYVMAKHTQ-UHFFFAOYSA-N); NSC743434 (ARZGLWFRVNUVDX-RIYZIHGNSA-N); NSC743696 (ZUGCFVKVDHHOMR-JMIUGGIZSA-N); NSC744020 (ZUCUXVICLPAASK-DEOSSOPVSA-N); NSC744327 (VRFNOEICKIYEAD-FIUWIIBXSA-N); NSC744330 (IFSWCDUZMRLVNK-XPGKHFPBSA-N); NSC744336 (JNOMYAFGRADGOL-CTLOQAHHSA-N); NSC744989 (VXLMCALNWIFZOP-RFYAYMHSSA-N); NSC745358 (MZTJKJHTFMPWQX-UHFFFAOYSA-N); NSC745632 (VOICQWAETDJKNM-KYJUHHDHSA-N); NSC745943 (HKEDBKXRDHFCFB-YERWJWFHSA-N); NSC749673 (ZCBYCWITATWYRM-UHFFFAOYSA-N); NSC750440 (NDOXBXQOMXCQJK-ZFMKNYSTSA-N); NSC750441 (OSKWPXWEGHYFGQ-BHVDBZJUSA-N); NSC750634 ion (MAWPZHTVBDJQKE-UHFFFAOYSA-Q) salts; NSC750854 (DTLIZHPZNQSZFO-UHFFFAOYSA-N); NSC751281 (WRPLJTYNAMMOEE-MIVNFNTKSA-N); NSC751642 (NFUVUOYLXYYFGJ-NJXSKGHCSA-N); NSC751817 (FWMCYVIWSSYEFA-UHFFFAOYSA-M); NSC751818 (FNCCNOYEBYFMMQ-UHFFFAOYSA-M); NSC751819 (RGTMFVSUDYTFHZ-UHFFFAOYSA-M); NSC751958 ion (PQOAWAWAVCOSLZ-NQPWKMLKSA-N) salts; NSC752270 (SRZOFNPQHMBPET-UHFFFAOYSA-N); NSC752271 (HFPJXGIKBRUERV-UHFFFAOYSA-N); NSC752272 (IJBXIFBSQZLFQM-UHFFFAOYSA-N); NSC752273 (HXAFCSVYXJOSKU-UHFFFAOYSA-N); NSC752274 (DIWBOPQXNJSWFF-UHFFFAOYSA-N); NSC752276 (RDOIPKDVLTUZNV-UHFFFAOYSA-N); NSC752278 (IRWJVGHILOFKQO-UHFFFAOYSA-N); NSC7523 (LSMIOFMZNVEEBR-UHFFFAOYSA-N); NSC752358 (GDYLQHMDBBDTLN-QFEZKATASA-N); NSC752464 (HAXYQNFQUDHACW-FIQZBLEBSA-N); NSC7525 (SANIPSLLLIEJOB-NCQVURFFSA-N); NSC752503 (ZBLSAIQQBQDYHA-UHFFFAOYSA-N); NSC752504 (MJOCBQIVFCWVAQ-UHFFFAOYSA-N); NSC752837 ion (MLAGCKQQXHHVMB-UHFFFAOYSA-M) salts; NSC753428 (RBOVXTWLIIRDMP-UHFFFAOYSA-N); NSC753601 (YYQHKAJBNHVYSL-UHFFFAOYSA-N); NSC753662 (UPCAIKXYYKGYLJ-WPTDRQDKSA-N); NSC753663 (LPZQKQWOQBJDOF-VVTVMFAVSA-N); NSC753664 (WXICJNNYNGCHPJ-YNYSCKANSA-N); NSC753665 (YRSPFSKBUWFBBW-SDKPGJOKSA-N); NSC753666 (IFBGRJRYJDVQLD-GRWWMUSUSA-N); NSC754152 (YEJMCBPDORCDTR-UHFFFAOYSA-N); NSC754153 (FAYJXKLTAICLII-UHFFFAOYSA-N); NSC754311 (FIZXYSKSWDZADX-YQMKNSBWSA-N); NSC755117 (JYBJHILCJYZCGV-ZROIWOOFSA-N); NSC755120 (FVZUAZCRTDVOQP-DHDCSXOGSA-N); NSC757145 (BCLOHFYDGKVJBU-STZFKDTASA-N); NSC757926 (HBLMAHQPUHUQJR-JMQWPVDRSA-N); NSC757927 (RDRVIUAYIZHUKD-DDEWIXKASA-N); NSC761151 (LXENAZOCDGGYRJ-MYBIQHOXSA-N); NSC761582 (NCGOMPSMYUGVQC-WSGDVLFSSA-N); NSC762045 (LHAPWXCQRKSNOH-ZZUDIIJMSA-N); NSC762781 (AHLMURYDGXTCCV-AOZNSOTRSA-N); NSC762848 (MOHPUIQBYKQHCJ-JEAUJHOTSA-N); NSC763372 (AAADJKPSAGHJNF-MKICQXMISA-N); NSC763373 (AICWVPUWQRFZDN-PWHKKFIBSA-N); NSC763729 (PKKMUEANKWVMET-UNOMPAQXSA-N); NSC765406 (VBLTYVJENWMRPE-XXVWQQKBSA-N); NSC766103 (NBWLTPLUWUQXDF-UHFFFAOYSA-N); NSC766216 (GDDMAHMUOIYBJS-TVGQLCNQSA-N); NSC766905 (NXMZRTZSMAMOFH-XRFSQDKESA-N); NSC766922 (UUSCWIGUMPDFDE-UHFFFAOYSA-L); NSC767781 (GFMAGLUIIRRGQY-MYYYXRDXSA-N); NSC767782 (AUBANKFMHKEWTH-XYGWBWBKSA-N); NSC767783 (PYDNXNARGKEQDC-QQXSKIMKSA-N); NSC767784 (BQOIEDOSLXOHSO-QQXSKIMKSA-N); NSC76919 (SQYHNFNEPRVUKC-UHFFFAOYSA-N); NSC77038 (QBQXQYSJPWXZJL-UHFFFAOYSA-N); NSC774816 ion (KSKVHWLBKCHCJK-UHFFFAOYSA-N) salts; NSC775507 (PFJQECMDAFLTOX-XRFSQDKESA-N); NSC775763 (UJWLAEMYBNPDMI-VAJFZCPGSA-N); NSC775978 (CXPSBTZKZXYZNU-UHFFFAOYSA-N); NSC776999 (RPGWLEZIJQWSOZ-PKTZIBPZSA-N); NSC777486 (XPMLDKVBJLRJJO-UHFFFAOYSA-N); NSC778436 (ZXBJORKVVITKOZ-UHFFFAOYSA-N); NSC778438 (TXZCVDVMSSUSFX-UHFFFAOYSA-N); NSC778439 (RBVPOITYPIPPMO-UHFFFAOYSA-N); NSC778441 (YVEFYWRMXOWVOA-UHFFFAOYSA-N); NSC780453 (ZXQJLBCJKMFMOL-HGXNGHEYSA-N); NSC780454 (SQAFSVRMMTVAFE-FEDLORSXSA-N); NSC780455 (HBSFCZMOLYSUFM-KDZHNWKMSA-N); NSC780456 (IYNFJJZHCUBHPC-ZKDCJRNVSA-N); NSC780457 (FYIRQUFQXRVBQV-DIJMLNEKSA-N); NSC780458 (HDFPUIGVWMMVMM-KRZOGROPSA-N); NSC780459 (WHRHJGQUYQCUGP-GPWMUCFLSA-N); NSC780462 (NCZWUEWKLCSUNO-CXPSBOMVSA-N); NSC780957 (UIZNRSGQTXFMOF-UHFFFAOYSA-N); NSC780958 (OAEWOLLUOVFYIT-UHFFFAOYSA-N); NSC781094 (KHCSBCXYWDALIA-YKSAKOAQSA-N); NSC781097 (WPGMOQSEFMSICF-YKSAKOAQSA-N); NSC781098 (XMEFVYAGPCUDCB-JXPYRENWSA-N); NSC781099 (UDVTWMRUJQNOOC-JXPYRENWSA-N); NSC781406 (BBRINJUWZRLWKK-UHFFFAOYSA-N); NSC781434 (ONLANXSSYRPCDT-UHFFFAOYSA-N); NSC781435 (DFEIUYXRXYRPAN-UHFFFAOYSA-N); NSC781466 (VVBIPKURTKHKCV-HCQXNYSJSA-N); NSC781471 (ZQGOZVXWQFXQFT-SUVQGPRRSA-N); NSC781520 (YWBXGXQCSWWXJB-PDSXEYIOSA-N); NSC781522 (FMGXTLBJBMKAAM-ZSDSOXJFSA-N); NSC781615 (MAXHHERSVIWPEB-UHFFFAOYSA-N); NSC781616 (OMZFTTFKAYXMCW-UHFFFAOYSA-N); NSC781666 (DOZCBFMWNWUULK-VULFUBBASA-N); NSC781667 (NJUXKBYXWJEBPK-JFLMPSFJSA-N); NSC781933 (YURWFEUKIWRRBK-UHFFFAOYSA-N); NSC782189 (KXVDXRHYVTZOCH-KKLWQCETSA-N); NSC782378 (LGVDMCVXHGKAMQ-UHFFFAOYSA-N); NSC782392 (BYRUSBJZFJEJLW-ADFDXATNSA-N); NSC782393 (KFUFVQHVACJVSE-RSONUFKPSA-N); NSC782856 (PFLDFIVHROLKQI-YQRKEEQESA-N); NSC783041 (WMCWJEWQJMPKCE-UHFFFAOYSA-N); NSC783218 (HSGHAUATLWSEIF-XKWKRSCCSA-N); NSC783219 (NNDWHWTXOMOPFE-SQOKJJRSSA-N); NSC783229 (HEXJQWUHJDNJFJ-UHFFFAOYSA-N); NSC783297 (OZJCVQRWLDUFPA-YVFWWETGSA-N); NSC783298 (KFNMIACXDPOGJG-KBBWBNCFSA-N); NSC783299 (WVZCVUIMRBCDPW-WAMQQGDBSA-N); NSC783628 (CTZVILBIWMAEAO-UHFFFAOYSA-N); NSC783640 (XMPVSJJNICFOPA-UHFFFAOYSA-N); NSC783641 (VRTPJMAJOZKENJ-UHFFFAOYSA-N); NSC783799 (JICWBGYOXWDBPV-UHFFFAOYSA-N); NSC784230 (HVOMWIHGTVBZEM-UHFFFAOYSA-N); NSC784288 (WOKJYCSOXKXAIU-UHFFFAOYSA-N); NSC784289 (SQMZYGAERPETAS-UHFFFAOYSA-N); NSC785026 (RGBMUBAQFAVBSQ-UHFFFAOYSA-N); NSC785389 (XOIMXGXGGAWIAO-ABACMYBCSA-N); NSC785395 (VEIDTAJLPNVATH-WWYUTXSFSA-N); NSC787553 (HLNFJUMBEWNQDW-UHFFFAOYSA-N); NSC787554 (XJUMGECKQMPEPJ-UHFFFAOYSA-N); NSC787555 (MYKLWNHUJQJTLG-UHFFFAOYSA-N); NSC787772 (DLVBJFSGRMMFDS-HFHAMQQBSA-N); NSC787774 (DLVBJFSGRMMFDS-MVKJJVDBSA-N); NSC787808 (VKXOOLULFNYFNL-QRVIBDJDSA-N); NSC787809 (MCAJHHIMUMZGAC-ZHZULCJRSA-N); NSC787810 (AHVIZCSRZSGHRH-WQRHYEAKSA-N); NSC787814 (WQESIZIROXOHNS-OYKKKHCWSA-N); NSC787815 (USTAAUUJWZWDMI-DXJNIWACSA-N); NSC787817 (ZODDUABDZOMAPC-UCQKPKSFSA-N); NSC787877 (CDBBTQHVVMUBTD-MHAVOLJWSA-N); NSC787957 (IQPDEJBOMPMIEQ-CJLVFECKSA-N); NSC788055 (NRHNUTNOWPUEOX-JIAAILLZSA-N); NSC788056 (PIAGQWIQDZAFDY-JFDOVZFRSA-N); NSC788161 (YLMHQPLSGVCFCO-UHFFFAOYSA-N); NSC788200 (GOECNPWEAHFRPL-UHFFFAOYSA-N); NSC788356 (LNCHPDRGFYTFCN-UHFFFAOYSA-N); NSC788586 (DXHAESYIXMABOA-UDUYQSOBSA-N); NSC788710 (AQEHZRJUUXYELN-UHFFFAOYSA-N); NSC788712 (IKISRXNVZCUHAM-UHFFFAOYSA-N); NSC788725 (ZWVKSWXYGXZVSL-ZDQYYBRASA-N); NSC788816 (IDOZAZATJGCQJE-ZYMOGRSISA-N); NSC788817 (SPTYKJSYNPOTDC-WTQRLHSKSA-N); NSC788818 (KTBCTQSJQQXDMB-TZHYSIJRSA-N); NSC788819 (SXXFGTCCPRGGCJ-WTQRLHSKSA-N); NSC788820 (NEWUFNXTKKEDNA-TZHYSIJRSA-N); NSC789120 (JXWNGQYVYDAIKJ-QFIPXVFZSA-N); NSC789275 (CMMMDTNBMHRIGG-UHFFFAOYSA-N); NSC789277 (YXJQXCGICBEGBL-UHFFFAOYSA-N); NSC789645 (CVBRQWNGZNXYHR-KKYHWDRJSA-N); NSC789646 (SPQNVRNQSIYXEO-GGFUHWEBSA-N); NSC789944 (OSERMIPXNLXAPD-BMJPDPRUSA-N); NSC789949 (WWUVMHRJRCRFSL-NRXKGJIXSA-N); NSC790634 (VPOIJBBUEJYXGJ-UHFFFAOYSA-N); NSC790650 (HAFDWUTUTAKIMQ-IDLUQQOTSA-N); NSC790909 (KRUKLNWJDVMOKH-HSECVZGPSA-N); NSC791000 (MNCQJKBLIYSVOV-AXYDXROSSA-N); NSC791048 (AEXUAHIBNSABRV-ISKFKSNPSA-N); NSC791143 (XEIMKDVBGIGNDT-XUVHLHGVSA-N); NSC791145 (SWCKSLZJWHNNPE-INJOPXNBSA-N); NSC791208 (JFACETXYABVHFD-ZAZHENERSA-N); NSC791267 (JXYBYMYHURFRKT-LBJPWWINSA-N); NSC791275 (YAQNUZLIGFNDOW-UHFFFAOYSA-N); NSC791326 ion (CSRFYMKBBCKSKH-UHFFFAOYSA-M) salts; NSC791327 ion (UTTPUEKFCIGRKU-UHFFFAOYSA-M) salts; NSC791346 (SOIAVARDRLXKJF-UHFFFAOYSA-N); NSC791347 (HXVVXKNTBSIBCM-UHFFFAOYSA-N); NSC791348 (IQTUJPHUXIBZEK-UHFFFAOYSA-N); NSC791349 (QTFVWAWZVNFZMJ-UHFFFAOYSA-N); NSC791526 (VEPPKJWJLBMKNF-YXRASNILSA-N); NSC791529 (PYEMJSXCJKFZTH-UHFFFAOYSA-N); NSC791540 (FTXVHRXCHNRRMP-UHFFFAOYSA-N); NSC791789 (CPWQWGDOHLEHPO-HVGPQAHESA-N); NSC791790 (VGTZCVRFUUIIEA-UHFFFAOYSA-N); NSC793144 (XMPMIRDIIGGBON-HIKVSJAYSA-N); NSC793145 (ZNQCEWTVWZGCCE-HIKVSJAYSA-N); NSC793146 (IQTCJBYCRUBFPU-WIUKDWKASA-N); NSC793589 (KODDBDYGKNPKQX-MOSHPQCFSA-N); NSC794097 (ULDYQZNYZMFHLA-QXYUJABFSA-N); NSC794221 (AQVXAJHYIYOFEV-UHFFFAOYSA-N); NSC794222 (UIPIXYKGGAAOQX-UHFFFAOYSA-N); NSC794557 (CJTRVTYNJQVOAK-UHFFFAOYSA-N); NSC794946 (OLTNQCKDPZMTLV-OSGLLZLXSA-N); NSC795139 (KUFIVSUHEZRPPY-UHFFFAOYSA-N); NSC795464 (GAIUVLLBLNSBDL-UHFFFAOYSA-N); NSC796237 (XZGQRYLIJXQDJU-UHFFFAOYSA-N); NSC797537 (RQCCIMUOBKCYKK-UHFFFAOYSA-N); NSC797557 (DASCYEOOVZMXDS-UHFFFAOYSA-N); NSC797558 (LMRRZVWNVPZYNG-UHFFFAOYSA-N); NSC797768 (XJKTXWSMMWPNTP-UHFFFAOYSA-N); NSC797910 (XCPFAUNYESDMDK-PHRCFWATSA-N); NSC797911 (XCPFAUNYESDMDK-BNYJDTCASA-N); NSC797912 (QUIIRXKMXFNZAC-LHOSQHSLSA-N); NSC798262 (WQHYQXROJHBGHC-YUVMCWJMSA-N); NSC798780 (ZHOXESCAODGLFK-GPFIVKHLSA-N); NSC798823 (QOWMPMYLRAWRMR-UHFFFAOYSA-N); NSC799248 (LAQZOUNYGVOUIZ-PIQRGQFYSA-N); NSC799251 (CGBJGSAEHDSALD-MWDRSEEBSA-N); NSC799252 (DNNIYTHOENIHJD-IJWWBIQNSA-N); NSC799253 (AWYFOSGPUUXKOH-FTHBNIPZSA-N); NSC799254 (HOQWGMYKYIQGGS-IJWWBIQNSA-N); NSC799650 (AOUKPCAVTZURHO-UHFFFAOYSA-N); NSC799916 (IHBXYOHDFDUHJE-UHFFFAOYSA-N); NSC800287 (NFZSCIGNYNUEPW-UHFFFAOYSA-N); NSC800288 (VRLWTLXADZKMHY-UHFFFAOYSA-N); NSC800349 (RJNWVGRGXKDNDY-UHFFFAOYSA-N); NSC800381 (RATBIMAQZWTQOG-UHFFFAOYSA-N); NSC801657 (YILZDELPUIJTQG-QYPKZVLMSA-N); NSC802076 (SSKUBXWRQZAJTR-UHFFFAOYSA-N); NSC802542 (UHZAKLGSRFIJQL-UHFFFAOYSA-N); NSC802545 (ATUNJVMZHZUMLV-UHFFFAOYSA-N); NSC803349 (GJROGSMMNLIADD-UHFFFAOYSA-N); NSC803713 (PXIFGRYDHZKICB-LJQANCHMSA-N); NSC804094 (OKTKDIKXOONFJR-UGACXFTFSA-N); NSC804219 (NWXJPZXGSSWFTA-UHFFFAOYSA-N); NSC804230 (PGNAJQRFHHABPN-PMPSAXMXSA-N); NSC804765 (SOUSLJWTOPNNSQ-UHFFFAOYSA-N); NSC805062 (DZUVIDDUUZODCZ-YHARCJFQSA-N); NSC805063 (RDLWSUOMJSFXKE-AYKLPDECSA-N); NSC805066 (NEIPOFLOSRATCT-RNIAWFEPSA-N); NSC805261 (RWYBERRBPYZNOB-UHFFFAOYSA-N); NSC805338 ion (MNYZWUYMOKPOFO-UMQBVPBWSA-N) salts; NSC805523 (FSYBHTLZKICEMM-UHFFFAOYSA-N); NSC805524 (ZCSYJPLCFGATCR-UHFFFAOYSA-N); NSC805531 (CIQPFGXIZMKWEQ-UHFFFAOYSA-N); NSC805637 (HPEGAZVUTLUIDJ-WXSATGQTSA-N); NSC805641 (OTRGBWOHMJOGNC-CCEZHUSRSA-N); NSC805650 (AOXCCMOMYAVXRB-FMIVXFBMSA-N); NSC805657 (LOZMNVRDYDXMBL-VAWYXSNFSA-N); NSC805745 (KKYIOSDFJOWSPU-UHFFFAOYSA-N); NSC806936 (SABFWXDEMUZZIJ-DHUJRADRSA-N); NSC808307 (GCXWMZOMTBHROK-UHFFFAOYSA-N); NSC808547 (WABAXDPJOLMMBL-LJAQVGFWSA-N); NSC808548 (CILHGIPPOCNVBN-UHFFFAOYSA-N); NSC808685 (GUQFXCVHRLPSGN-UHFFFAOYSA-N); NSC809043 (SOLCIDNLQPAOAV-UHFFFAOYSA-N); NSC809044 (AZJMZAPPIGGBBR-UHFFFAOYSA-N); NSC809141 (MTGFMPZWABNBKK-UHFFFAOYSA-N); NSC809150 (COYDAQRGZBCOGM-UHFFFAOYSA-N); NSC809699 (HUVFKXVHSAFFKN-UHFFFAOYSA-N); NSC809841 (MYAQMZRLNNWEQR-GHXNOFRVSA-N); NSC809842 (KDVCLHFQQKFLBJ-XFXZXTDPSA-N); NSC809870 (SAHXMFBYTCHZMB-XFXZXTDPSA-N); NSC809947 (UIZKUPNJEZHWDY-UVTDQMKNSA-N); NSC809975 (DZZPMJSZDUQBHX-BHVANESWSA-N); NSC809976 (LKJSSDDTLKPNSQ-BHVANESWSA-N); NSC810107 (UNGMATJCAUDGQM-UHFFFAOYSA-N); NSC810108 (JNBXSGJNHKFHBM-UHFFFAOYSA-N); NSC810311 (GWSOVEKHFSFSLB-UHFFFAOYSA-N); NSC810313 (YWJOANWTDWDVNM-UHFFFAOYSA-N); NSC810445 (QKXMCXBQIYHTTB-UHFFFAOYSA-N); NSC810447 (ZDJBDCRRUYTAMF-UHFFFAOYSA-N); NSC810564 (VMWCRPBJOWRRBW-UHFFFAOYSA-N); NSC811013 (KJUIFNUQKGPZIL-ZDLGFXPLSA-N); NSC811246 (KHLGSZUFWZZPMW-UHFFFAOYSA-N); NSC811260 (ZFAOMUNOLMSOTF-UHFFFAOYSA-N); NSC811265 (QCYVCDNWIYUGCZ-UHFFFAOYSA-N); NSC811273 (QDWFEOGQXMJIKH-UHFFFAOYSA-N); NSC811274 (BXILTTCLNXGVPZ-UHFFFAOYSA-N); NSC811276 (PGTAXJRIAPPCST-UHFFFAOYSA-N); NSC811879 ion (FICRYFXUIRKMMG-UHFFFAOYSA-N) salts; NSC812049 (FUSSHCHWIOWJGZ-UHFFFAOYSA-N); NSC812050 (SCCNGNDWRDDOGO-UHFFFAOYSA-N); NSC812160 (XSAATKJHMTWAGM-UHFFFAOYSA-N); NSC812161 (YUMXDPRSDIMRRA-UHFFFAOYSA-N); NSC812733 (KWESBUSERYSVGB-UHFFFAOYSA-N); NSC812738 (NCYPELKKABGTPV-UHFFFAOYSA-N); NSC813204 (AFGJDCFUEWTJNA-HNNXBMFYSA-N); NSC813206 (MSHZZYFWEVYFNT-KRWDZBQOSA-N); NSC813208 (BLSKEIGIPYWZAQ-HNNXBMFYSA-N); NSC813209 (IJHORLIDBBFFOG-AWEZNQCLSA-N); NSC813210 (SYTZWTUQGGPTSG-INIZCTEOSA-N); NSC813501 (SQDALODCMFSPNR-UHFFFAOYSA-N); NSC813647 (VTSIWIUPMZQLJS-UHFFFAOYSA-N); NSC813650 (MTVMFRKFDIVFRS-UHFFFAOYSA-N); NSC814062 ion (FJCGPUPBERXVMC-UHFFFAOYSA-N) salts; NSC814068 (LUTUQLHBWXKFOT-UHFFFAOYSA-M); NSC814069 ion (NZEBHOOKVLDJAQ-UHFFFAOYSA-N) salts; NSC814343 (JLPCHFBZEQPJPE-UHFFFAOYSA-N); NSC814602 (HYJPRAXGHYXJGG-UHFFFAOYSA-N); NSC815062 (HBOGRCGQQODIIP-SDNWHVSQSA-N); NSC815065 (QRDIWWOOHJXKJL-SDNWHVSQSA-N); NSC815386 (WCLKJUWBJSHIMO-UHFFFAOYSA-N); NSC815387 (CCSUURLUZYEROA-UHFFFAOYSA-N); NSC815401 (FKHNDIHEUMXYJS-UHFFFAOYSA-N); NSC815402 (NZAVORYSPNSKMY-UHFFFAOYSA-N); NSC815403 (CUKYLQLHKFZACV-UHFFFAOYSA-N); NSC815404 (UFVQQAIFPBGUIJ-UHFFFAOYSA-N); NSC815405 (CHINNXALMYRLII-UHFFFAOYSA-N); NSC815561 (DBWHJJJEXSKVJF-WUKNDPDISA-N); NSC816001 ion (GANGHEKFXWEFHC-UHFFFAOYSA-P) salts; NSC816003 ion (UUDCFQUUGPPBQV-UHFFFAOYSA-P) salts; NSC816090 (TWRQFLCIKXWUTJ-UHFFFAOYSA-N); NSC816184 (NWMGNBXSJSNRHM-UHFFFAOYSA-N); NSC816968 ion (IGHGYYMGUHJICH-UHFFFAOYSA-N) salts; NSC816969 ion (VNEWUGYUAJSIJZ-UHFFFAOYSA-N) salts; NSC98579 (AKDWTVCYSZSYQZ-UHFFFAOYSA-N); NVP-HSP990 (WSMQUUGTQYPVPD-OAHLLOKOSA-N); Nadifloxacin (JYJTVFIEFKZWCJ-UHFFFAOYSA-N); Naftifine hydrochloride (OLUNPKFOFGZHRT-YGCVIUNWSA-N); Naftopidil (HRRBJVNMSRJFHQ-UHFFFAOYSA-N); Naltrexone (DQCKKXVULJGBQN-XFWGSAIBSA-N); Nandrolone (NPAGDVCDWIYMMC-IZPLOLCNSA-N); Nandrolone decanoate (JKWKMORAXJQQSR-MOPIKTETSA-N); Nandrolone phenpropionate (UBWXUGDQUBIEIZ-QNTYDACNSA-N); Napabucasin (DPHUWDIXHNQOSY-UHFFFAOYSA-N); Naphazoline (CNIIGCLFLJGOGP-UHFFFAOYSA-N); Naphazoline hydrochloride (DJDFFEBSKJCGHC-UHFFFAOYSA-N); Naphtho[1′,2′:4,5]imidazo[1,2-a]pyrimidine-5,6-dione (GKTWMKZOHJRQNH-UHFFFAOYSA-N); Naporafenib (UEPXBTCUIIGYCY-UHFFFAOYSA-N); Naquotinib (QKDCLUARMDUUKN-XMMPIXPASA-N); Naratriptan (AMKVXSZCKVJAGH-UHFFFAOYSA-N); Narciclasine (LZAZURSABQIKGB-AEKGRLRDSA-N); Naringenin (FTVWIRXFELQLPI-ZDUSSCGKSA-N); Navelbine (GBABOYUKABKIAF-IELIFDKJSA-N); Navitoclax (JLYAXFNOILIKPP-KXQOOQHDSA-N); Navoximod (YGACXVRLDHEXKY-LHPNLFKDSA-N); Naxcel (ZBHXIWJRIFEVQY-IHMPYVIRSA-N); Nazartinib (IOMMMLWIABWRKL-WUTDNEBXSA-N); Nebivolol hcl (JWEXHQAEWHKGCW-BIISKSHESA-N); Nedisertib (MOWXJLUYGFNTAL-DEOSSOPVSA-N); Nefazodone hydrochloride (DYCKFEBIOUQECE-UHFFFAOYSA-N); Neflamapimod (VEPKQEUBKLEPRA-UHFFFAOYSA-N); Nelarabine (IXOXBSCIXZEQEQ-UHTZMRCNSA-N); Nelfinavir (QAGYKUNXZHXKMR-HKWSIXNMSA-N); Nelfinavir mesylate (NQHXCOAXSHGTIA-SKXNDZRYSA-N); Neohesperidin dihydrochalcone (ITVGXXMINPYUHD-CUVHLRMHSA-N); Neolymphostin a (AMBGZHQTMCPRDB-SSQJTDSESA-N); Neooxypate ion (ZFWFZMYQXOKWIY-UHFFFAOYSA-L) salts; Nepafenac (QEFAQIPZVLVERP-UHFFFAOYSA-N); Nepicastat (YZZVIKDAOTXDEB-JTQLQIEISA-N); Neratinib (JWNPDZNEKVCWMY-VQHVLOKHSA-N); Nesbuvir (WTDWVLJJJOTABN-UHFFFAOYSA-N); Netarsudil (OURRXQUGYQRVML-AREMUKBSSA-N); Neticonazole (VWOIKFDZQQLJBJ-DTQAZKPQSA-N); Nevirapine (NQDJXKOVJZTUJA-UHFFFAOYSA-N); Nialamide (NOIIUHRQUVNIDD-UHFFFAOYSA-N); Nicardipine (ZBBHBTPTTSWHBA-UHFFFAOYSA-N); Nickeldibutyldithiocarbamate (BOXSVZNGTQTENJ-UHFFFAOYSA-L); Niclosamide (RJMUSRYZPJIFPJ-UHFFFAOYSA-N); Nicorandil (LBHIOVVIQHSOQN-UHFFFAOYSA-N); Nicotinamide (DFPAKSUCGFBDDF-UHFFFAOYSA-N); Nicotine (SNICXCGAKADSCV-JTQLQIEISA-N); Nicotinic acid (PVNIIMVLHYAWGP-UHFFFAOYSA-N); Nifedipine (HYIMSNHJOBLJNT-UHFFFAOYSA-N); Niflumic acid (JZFPYUNJRRFVQU-UHFFFAOYSA-N); Nifuroxazide (YCWSUKQGVSGXJO-NTUHNPAUSA-N); Nifurquinazol (AUEOHSUMWXAPBX-UHFFFAOYSA-N); Niguldipine hydrochloride (MHOSUIMBPQVOEU-UHFFFAOYSA-N); Nile Blue 2B (FAJDWNKDRFAWLS-UHFFFAOYSA-N); Nile blue chloride (SHXOKQKTZJXHHR-UHFFFAOYSA-N); Nilotinib (HHZIURLSWUIHRB-UHFFFAOYSA-N); Nilutamide (XWXYUMMDTVBTOU-UHFFFAOYSA-N); Nilvadipine (FAIIFDPAEUKBEP-UHFFFAOYSA-N); Nimesulide (HYWYRSMBCFDLJT-UHFFFAOYSA-N); Nimodipine (UIAGMCDKSXEBJQ-UHFFFAOYSA-N); Nimustine (VFEDRRNHLBGPNN-UHFFFAOYSA-N); Nimustine hydrochloride (KPMKNHGAPDCYLP-UHFFFAOYSA-N); Nintedanib (CPMDPSXJELVGJG-UHFFFAOYSA-N); Niraparib (PCHKPVIQAHNQLW-CQSZACIVSA-N); Nirogacestat (VFCRKLWBYMDAED-REWPJTCUSA-N); Nisoldipine (VKQFCGNPDRICFG-UHFFFAOYSA-N); Nitarsone (FUUFQLXAIUOWML-UHFFFAOYSA-N); Nitazoxanide (YQNQNVDNTFHQSW-UHFFFAOYSA-N); Nitidine chloride (QLDAACVSUMUMOR-UHFFFAOYSA-M); Nitisinone (OUBCNLGXQFSTLU-UHFFFAOYSA-N); Nitracrine dihydrochloride (BKBNOPIWNDASON-UHFFFAOYSA-N); Nitrendipine (PVHUJELLJLJGLN-UHFFFAOYSA-N); Nitrofurantoin (NXFQHRVNIOXGAQ-YCRREMRBSA-N); Nitrofurazone (IAIWVQXQOWNYOU-FPYGCLRLSA-N); Nitroxoline (RJIWZDNTCBHXAL-UHFFFAOYSA-N); Nocodazole (KYRVNWMVYQXFEU-UHFFFAOYSA-N); Nogalomycin c (NNEGMXMRXKXKMQ-UHFFFAOYSA-N); Nolatrexed (XHWRWCSCBDLOLM-UHFFFAOYSA-N); Nolvasan (GHXZTYHSJHQHIJ-UHFFFAOYSA-N); Nomifensine maleate (GEOCVSMCLVIOEV-BTJKTKAUSA-N); Nonivamide (RGOVYLWUIBMPGK-UHFFFAOYSA-N); Nonoxynol-9 (FBWNMEQMRUMQSO-UHFFFAOYSA-N); Nordihydroguaiaretic acid (HCZKYJDFEPMADG-UHFFFAOYSA-N); Norepinephrine (SFLSHLFXELFNJZ-QMMMGPOBSA-N); Norethindrone (VIKNJXKGJWUCNN-XGXHKTLJSA-N); Norethindrone acetate (IMONTRJLAWHYGT-ZCPXKWAGSA-N); Norethisterone enanthate (APTGJECXMIKIET-WOSSHHRXSA-N); Norethynodrel (ICTXHFFSOAJUMG-SLHNCBLASA-N); Norgestrel (WWYNJERNGUHSAO-XUDSTZEESA-N); Nornicotine (MYKUKUCHPMASKF-UHFFFAOYSA-N); Nortriptyline (PHVGLTMQBUFIQQ-UHFFFAOYSA-N); Nortriptyline hydrochloride (SHAYBENGXDALFF-UHFFFAOYSA-N); Noscapine (AKNNEGZIBPJZJG-MSOLQXFVSA-N); Nps-2143 (PZUJQWHTIRWCID-HXUWFJFHSA-N); Nutlin-3 (BDUHCSBCVGXTJM-UHFFFAOYSA-N); Nvp-aew541 (AECDBHGVIIRMOI-UHFFFAOYSA-N); Nvp-cgm097 (CLRSLRWKONPSRQ-CPOWQTMSSA-N); Nylidrin (PTGXAUBQBSGPKF-UHFFFAOYSA-N); 0,0′-Diacetylmorphine hydrochloride (FZJYQGFGNHGSFX-PVQKIFDLSA-N); O-(chloroacetylcarbamoyl)fumagillol (MSHZHSPISPJWHW-PVDLLORBSA-N); ODM-203 (ZJFCBQXPTQSTCZ-UHFFFAOYSA-N); ON123300 (VADOZMZXXRBXNY-UHFFFAOYSA-N); ONC212 (DFULPGUTXZTYKA-UHFFFAOYSA-N); OTS514 (OETLNMOJNONWOY-LBPRGKRZSA-N); Obatoclax (CVCLJVVBHYOXDC-IAZSKANUSA-N); Obatoclax mesylate (ZVAGBRFUYHSUHA-RKMKAUCSSA-N); Octocrylene (FMJSMJQBSVNSBF-UHFFFAOYSA-N); Octodrine (QNIVIMYXGGFTAK-UHFFFAOYSA-N); Octyl gallate (NRPKURNSADTHLJ-UHFFFAOYSA-N); Odanacatib (FWIVDMJALNEADT-SFTDATJTSA-N); Ofloxacin (GSDSWSVVBLHKDQ-UHFFFAOYSA-N); Olanzapine (KVWDHTXUZHCGIO-UHFFFAOYSA-N); Olaparib (FDLYAMZZIXQODN-UHFFFAOYSA-N); Oleic acid (ZQPPMHVWECSIRJ-KTKRTIGZSA-N); Olmutinib (FDMQDKQUTRLUBU-UHFFFAOYSA-N); Oltipraz (CKNAQFVBEHDJQV-UHFFFAOYSA-N); Olutasidenib (NEQYWYXGTJDAKR-JTQLQIEISA-N); Omacetaxine mepesuccinate (HYFHYPWGAURHIV-JFIAXGOJSA-N); Omaveloxolone (RJCWBNBKOKFWNY-IDPLTSGASA-N); Ombrabulin (IXWNTLSTOZFSCM-YVACAVLKSA-N); Omecamtiv mecarbil (RFUBTTPMWSKEIW-UHFFFAOYSA-N); Omeprazole (SUBDBMMJDZJVOS-UHFFFAOYSA-N); Omipalisib (CGBJSGAELGCMKE-UHFFFAOYSA-N); Onalespib (IFRGXKKQHBVPCQ-UHFFFAOYSA-N); Onapristone (IEXUMDBQLIVNHZ-YOUGDJEHSA-N); Oncopore (GGAKLYWEFZCVIT-TVEKFXMRSA-N); Oncovin (AQTQHPDCURKLKT-PNYVAJAMSA-N); Ondansetron (FELGMEQIXOGIFQ-UHFFFAOYSA-N); Ondansetron hydrochloride dihydrate (VRSLTNZJOUZKLX-UHFFFAOYSA-N); Onvansertib (QHLVBNKYJGBCQJ-UHFFFAOYSA-N); Oprea1_106347 (ZDZIMAFZTGCFOH-UHFFFAOYSA-N); Oprea1_501737 (QRTCBAURJCNMAD-UHFFFAOYSA-N); Oprozomib (SWZXEVABPLUDIO-WSZYKNRRSA-N); Orantinib (NHFDRBXTEDBWCZ-ZROIWOOFSA-N); Orlistat (AHLBNYSZXLDEJQ-FWEHEUNISA-N); Orotic acid (PXQPEWDEAKTCGB-UHFFFAOYSA-N); Ortataxel (BWKDAMBGCPRVPI-ZQRPHVBESA-N); Ortho evra (ISHXLNHNDMZNMC-VTKCIJPMSA-N); Osi-027 (JROFGZPOBKIAEW-UHFFFAOYSA-N); Osi-930 (FGTCROZDHDSNIO-UHFFFAOYSA-N); Osilodrostat (USUZGMWDZDXMDG-CYBMUJFWSA-N); Osimertinib (DUYJMQONPNNFPI-UHFFFAOYSA-N); Osthole (MBRLOUHOWLUMFF-UHFFFAOYSA-N); Otenabant (UNAZAADNBYXMIV-UHFFFAOYSA-N); Otilonium bromide (VWZPIJGXYWHBOW-UHFFFAOYSA-N); Otilonium ion (NQHNLNLJPDMBFN-UHFFFAOYSA-O) salts; Otssp167 (DKZYXHCYPUVGAF-UHFFFAOYSA-N); Ouabain (LPMXVESGRSUGHW-HBYQJFLCSA-N); Oxalic acid (MUBZPKHOEPUJKR-UHFFFAOYSA-N); Oxatomide (BAINIUMDFURPJM-UHFFFAOYSA-N); Oxazepam (ADIMAYPTOBDMTL-UHFFFAOYSA-N); Oxcarbazepine (CTRLABGOLIVAIY-UHFFFAOYSA-N); Oxethazaine (FTLDJPRFCGDUFH-UHFFFAOYSA-N); Oxfendazole (BEZZFPOZAYTVHN-UHFFFAOYSA-N); Oxibendazole (RAOCRURYZCVHMG-UHFFFAOYSA-N); Oxiconazole nitrate (WVNOAGNOIPTWPT-NDUABGMUSA-N); Oxybutynin chloride (SWIJYDAEGSIQPZ-UHFFFAOYSA-N); Oxymetazoline (WYWIFABBXFUGLM-UHFFFAOYSA-N); Oxymetholone (ICMWWNHDUZJFDW-DHODBPELSA-N); Oxyphenbutazone (HFHZKZSRXITVMK-UHFFFAOYSA-N); Oxyphencyclimine hydrochloride (WXAYTPABEADAAB-UHFFFAOYSA-N); Oxyphenisatin acetate (PHPUXYRXPHEJDF-UHFFFAOYSA-N); Oxyphenisatine (SJDACOMXKWHBOW-UHFFFAOYSA-N); Oxytetracycline (OWFJMIVZYSDULZ-PXOLEDIWSA-N); Ozanimod (XRVDGNKRPOAQTN-FQEVSTJZSA-N); P-Ara-C(SHBAKEKBTCPUFI-UHFFFAOYSA-N); PAC1 (YQNRVGJCPCNMKT-JLPGSUDCSA-N); PCI-32765 Racemate (XYFPWWZEPKGCCK-UHFFFAOYSA-N); PD0166285 (IFPPYSWJNWHOLQ-UHFFFAOYSA-N); PDK1 inhibitor AR-12 (YULUCECVQOCQFQ-UHFFFAOYSA-N); PF-00562271 free base (MZDKLVOWGIOKTN-UHFFFAOYSA-N); PF-03814735 (RYYNGWLOYLRZLK-RBUKOAKNSA-N); PF-04217903 (PDMUGYOXRHVNMO-UHFFFAOYSA-N); PF-04457845 (BATCTBJIJJEPHM-UHFFFAOYSA-N); PF-04691502 (XDLYKKIQACFMJG-UHFFFAOYSA-N); PF-06459988 (ODMXWZROLKITMS-RISCZKNCSA-N); PF-06821497 (RXCVUHMIWHRLDF-HXUWFJFHSA-N); PF-3758309 (AYCPARAPKDAOEN-LJQANCHMSA-N); PF-477736 (NDEXUOWTGYUVGA-LJQANCHMSA-N); PFK-158 (IAJOMYABKVAZCN-AATRIKPKSA-N); PH-797804 (KCAJXIDMCNPGHZ-UHFFFAOYSA-N); PHA-680632 (OBWNXGOQPLDDPS-UHFFFAOYSA-N); PHA-793887 (HUXYBQXJVXOMKX-UHFFFAOYSA-N); PI-103 (TUVCWJQQGGETHL-UHFFFAOYSA-N); PIK-75 (QTHCAAFKVUWAFI-DJKKODMXSA-N); PIM447 (VRQXRVAKPDCRCI-ZNMIVQPWSA-N); PKI-179 (WXUUCRLKXQMWRY-UHFFFAOYSA-N); PKI-402 (ZAXFYGBKZSQBIV-UHFFFAOYSA-N); PLX-4720 (YZDJQTHVDDOVHR-UHFFFAOYSA-N); PLX51107 (AMSUHYUVOVCWTP-INIZCTEOSA-N); PLX8394 (YYACLQUDUDXAPA-MRXNPFEDSA-N); PND-1186 (IGUBBWJDMLCRIK-UHFFFAOYSA-N); PP121 (NVRXTLZYXZNATH-UHFFFAOYSA-N); PRN1371 (PUIXMSRTTHLNKI-UHFFFAOYSA-N); PRT-060318 (NZNTWOVDIXCHHS-LSDHHAIUSA-N); PRT062607 (TXGKRVFSSHPBAJ-JKSUJKDBSA-N); PRX-08066 free base (IENZFHBNCRQMNP-UHFFFAOYSA-N); PT-2385 (ONBSHRSJOPSEGS-INIZCTEOSA-N); PU-H71 (SUPVGFZUWFMATN-UHFFFAOYSA-N); Paclitaxel (RCINICONZNJXQF-MZXODVADSA-N); Paclitaxel (taxol) (RCINICONZNJXQF-VAZQATRQSA-N); Pacritinib (HWXVIOGONBBTBY-ONEGZZNKSA-N); Paeonol (UILPJVPSNHJFIK-UHFFFAOYSA-N); Pagoclone (HIUPRQPBWVEQJJ-UHFFFAOYSA-N); Pak4-IN-1 (MRFOPLWJZULAQD-SWGQDTFXSA-N); Palbociclib (AHJRHEGDXFFMBM-UHFFFAOYSA-N); Palbociclib hydrochloride (STEQOHNDWONVIF-UHFFFAOYSA-N); Palifosfamide (BKCJZNIZRWYHBN-UHFFFAOYSA-N); Palmatine chloride (RLQYRXCUPVKSAW-UHFFFAOYSA-M); Palomid 529 (YEAHTLOYHVWAKW-UHFFFAOYSA-N); Palonosetron hydrochloride (OLDRWYVIKMSFFB-SSPJITILSA-N); Pamicogrel (ISCHOARKJADAKJ-UHFFFAOYSA-N); Pamiparib (DENYZIUJOTUUNY-MRXNPFEDSA-N); Pancratistatin (VREZDOWOLGNDPW-ALTGWBOUSA-N); Panobinostat (FPOHNWQLNRZRFC-ZHACJKMWSA-N); Pantethine (DJWYOLJPSHDSAL-ROUUACIJSA-N); Pantoprazole (IQPSEEYGBUAQFF-UHFFFAOYSA-N); Pantoprazole sodium (YNWDKZIIWCEDEE-UHFFFAOYSA-N); Pantothenic acid (GHOKWGTUZJEAQD-ZETCQYMHSA-N); Papaverine (XQYZDYMELSJDRZ-UHFFFAOYSA-N); Papaverine hydrochloride (UOTMYNBWXDUBNX-UHFFFAOYSA-N); Parbendazole (YRWLZFXJFBZBEY-UHFFFAOYSA-N); Paricalcitol (BPKAHTKRCLCHEA-UBFJEZKGSA-N); Paroxetine (AHOUBRCZNHFOSL-YOEHRIQHSA-N); Paroxetine hydrochloride (GELRVIPPMNMYGS-RVXRQPKJSA-N); Parsaclisib (ZQPDJCIXJHUERQ-QWRGUYRKSA-N); Parthenolide (KTEXNACQROZXEV-PVLRGYAZSA-N); Parthenolide (KTEXNACQROZXEV-QLIGOWBFSA-N); Parthenolide (KTEXNACQROZXEV-SLXBATTESA-N); Pazopanib (CUIHSIWYWATEQL-UHFFFAOYSA-N); Pecazine (CBHCDHNUZWWAPP-UHFFFAOYSA-N); Pectamol (KVKJFNUGVOFNGU-UHFFFAOYSA-N); Pederin (ZNEZZONMADKYTB-VRCUBXEUSA-N); Pelcitoclax (QIOCQCYXBYUYLH-YACUFSJGSA-N); Pelitinib (WVUNYSQLFKLYNI-AATRIKPKSA-N); Pelitrexol (QXOPTIPQEVJERB-JQWIXIFHSA-N); Pemetrexed (WBXPDJSOTKVWSJ-ZDUSSCGKSA-N); Pemetrexed disodium (NYDXNILOWQXUOF-GXKRWWSZSA-L); Pemigatinib (HCDMJFOHIXMBOV-UHFFFAOYSA-N); Penbutolol sulfate (FEDSNBHHWZEYTP-ZFQYHYQMSA-N); Penciclovir (JNTOCHDNEULJHD-UHFFFAOYSA-N); Penclomedine (DZVPGIORVGSQMC-UHFFFAOYSA-N); Penfluridol (MDLAAYDRRZXJIF-UHFFFAOYSA-N); Peninsularinone (YYFUPACSPMCQHM-IAXDHZOFSA-N); Pentagastrin (NEYNJQRKHLUJRU-DZUOILHNSA-N); Pentamidine (XDRYMKDFEDOLFX-UHFFFAOYSA-N); Pentamidine isethionate (YBVNFKZSMZGRAD-UHFFFAOYSA-N); Pentetic acid (QPCDCPDFJACHGM-UHFFFAOYSA-N); Pepstatin (FAXGPCHRFPCXOO-LXTPJMTPSA-N); Perazine (WEYVCQFUGFRXOM-UHFFFAOYSA-N); Pergolide (YEHCICAEULNIGD-MZMPZRCHSA-N); Pergolide mesylate (UWCVGPLTGZWHGS-ZORIOUSZSA-N); Pergularinine (LAWAARLALKUFQQ-CVDCTZTESA-N); Perhexiline (CYXKNKQEMFBLER-UHFFFAOYSA-N); Perhexiline maleate (JDZOTSLZMQDFLG-BTJKTKAUSA-N); Periciazine (LUALIOATIOESLM-UHFFFAOYSA-N); Perifosine (SZFPYBIJACMNJV-UHFFFAOYSA-N); Perospirone hydrochloride (HIZFAPMOZFYELI-GNXQHMNLSA-N); Perphenazine (RGCVKNLCSQQDEP-UHFFFAOYSA-N); Pevonedistat (MPUQHZXIXSTTDU-QXGSTGNESA-N); Pexidartinib (JGWRKYUXBBNENE-UHFFFAOYSA-N); Pexmetinib (LNMRSSIMGCDUTP-UHFFFAOYSA-N); Pha-665752 (OYONTEXKYJZFHA-SSHUPFPWSA-N); Pharmakon1600-01500247 (LTMHDMANZUZIPE-QVYPYPLASA-N); Pharmakon1600-01500272 (HUEYSSLYFJVUIS-PBMRNQEBSA-N); Pharmakon1600-01500676 (LPMXVESGRSUGHW-XPXORLBYSA-N); Pharmakon1600-01501105 (MXJUOYXSYWPMAR-VWLXJURHSA-N); Pharmakon1600-01501205 (JAYAGJDXJIDEKI-YBYZECQXSA-N); Pharmakon1600-01502258 ion (GYPNBQUSNIBSGD-DUDHYVAZSA-N) salts; Pharmakon1600-01503223 (OILMTGAXBCPFHL-UHFFFAOYSA-N); Pharmakon1600-01503867 (HYIZCDMXFDRDFE-RTQJASBYSA-N); Pharmakon1600-01506084 (GIBKNVJEIILEKC-FBDKWHMPSA-N); Phenazine methosulfate (RXGJTUSBYWCRBK-UHFFFAOYSA-M); Phenazopyridine hydrochloride (QQBPIHBUCMDKFG-UHFFFAOYSA-N); Phenelzine (RMUCZJUITONUFY-UHFFFAOYSA-N); Phenformin (ICFJFFQQTFMIBG-UHFFFAOYSA-N); Phenformin hydrochloride (YSUCWSWKRIOILX-UHFFFAOYSA-N); Phenobarbital (DDBREPKUVSBGFI-UHFFFAOYSA-N); Phenolphthalein (KJFMBFZCATUALV-UHFFFAOYSA-N); Phenothrin (SBNFWQZLDJGRLK-UHFFFAOYSA-N); Phenoxybenzamine (QZVCTJOXCFMACW-UHFFFAOYSA-N); Phentolamine (MRBDMNSDAVCSSF-UHFFFAOYSA-N); Phenyl aminosalicylate (DNVVZWSVACQWJE-UHFFFAOYSA-N); Phenylbutazone (VYMDGNCVAMGZFE-UHFFFAOYSA-N); Phloroglucinol (QCDYQQDYXPDABM-UHFFFAOYSA-N); Phosmet (LMNZTLDVJIUSHT-UHFFFAOYSA-N); Phosphoramide mustard cyclohexylamine salt (BGTIPRUDEMNRIP-UHFFFAOYSA-N); Phyllanthastatin 1 (QJTFVHQEZWKQOM-SDNWHVSQSA-N); Phyllanthoside (VOTNXJVGRXZYOA-SDNWHVSQSA-N); Physostigmine (PIJVFDBKTWXHHD-HIFRSBDPSA-N); Pibenzimol hcl (UDJDXLXTHZQAAC-UHFFFAOYSA-N); Piboserod (KVCSJPATKXABRQ-UHFFFAOYSA-N); Picropodophyllin (YJGVMLPVUAXIQN-HAEOHBJNSA-N); Picropodophyllotoxin acetate (SASVNKPCTLROPQ-RGXPITOMSA-N); Pictilisib (LHNIIDJUOCFXAP-UHFFFAOYSA-N); Pilaralisib (QINPEPAQOBZPOF-UHFFFAOYSA-N); Pimasertib (VIUAUNHCRHHYNE-JTQLQIEISA-N); Pimavanserin (RKEWSXXUOLRFBX-UHFFFAOYSA-N); Pimethixene maleate (IPANUAHQWFDVAG-BTJKTKAUSA-N); Pimobendan (GLBJJMFZWDBELO-UHFFFAOYSA-N); Pimozide (YVUQSNJEYSNKRX-UHFFFAOYSA-N); Pinafide (GBOQUHPYCRYKGV-UHFFFAOYSA-N); Pinaverium bromide (IKGXLCMLVINENI-UHFFFAOYSA-M); Pinometostat (LXFOLMYKSYSZQS-XKHGBIBOSA-N); Pioglitazone (HYAFETHFCAUJAY-UHFFFAOYSA-N); Pipamperone (AXKPFOAXAHJUAG-UHFFFAOYSA-N); Piperacetazine (BTFMCMVEUCGQDX-UHFFFAOYSA-N); Piperazinedione 593A (DWDDRBRKQBGRAJ-ZARMYCFFSA-N); Piperidinium,1-didodecyl-3-methyl-, bromide (LQEPGIXKXOCKDY-UHFFFAOYSA-M); Piperidolate hydrochloride (RBGWCEWDAHDPEH-UHFFFAOYSA-N); Piperine (MXXWOMGUGJBKIW-YPCIICBESA-N); Piperonyl butoxide (FIPWRIJSWJWJAI-UHFFFAOYSA-N); Pipobroman (NJBFOOCLYDNZJN-UHFFFAOYSA-N); Pirarubicin (KMSKQZKKOZQFFG-YXRRJAAWSA-N); Piritrexim (VJXSSYDSOJBUAV-UHFFFAOYSA-N); Pirlindole mesylate (PVUYTBQAVAYLDE-UHFFFAOYSA-N); Piroxicam (QYSPLQLAKJAUJT-UHFFFAOYSA-N); Pitavastatin hemicalcium (RHGYHLPFVJEAOC-FFNUKLMVSA-L); Pizotifen (FIADGNVRKBPQEU-UHFFFAOYSA-N); Platinum, diamminedichloro-(DQLATGHUWYMOKM-UHFFFAOYSA-L); Platinum,2-cyclohexanediamine-N,N′)(sulfato(2-)-O,O′)-, (SP-4-2)-(IGQTWWNTYQYGTM-UHFFFAOYSA-N); Pleconaril (KQOXLKOJHVFTRN-UHFFFAOYSA-N); Plerixafor (YIQPUIGJQJDJOS-UHFFFAOYSA-N); Plerixafor octahydrochloride (UEUPDYPUTTUXLJ-UHFFFAOYSA-N); Plevitrexed (IEJSCSAMMLUINT-NRFANRHFSA-N); Plicamycin (CFCUWKMKBJTWLW-BKHRDMLASA-N); Plinabulin (UNRCMCRRFYFGFX-TYPNBTCFSA-N); Plumbagin (VCMMXZQDRFWYSE-UHFFFAOYSA-N); Plumbane, tripropyl[(trifluoroacetyl)oxy]-(YUJDDCSEPYWVPK-UHFFFAOYSA-M); Pluripotin (NBZFRTJWEIHFPF-UHFFFAOYSA-N); Podofilox (YJGVMLPVUAXIQN-XVVDYKMHSA-N); Podophyllotoxin bromide (VNDCSULMOTZMNT-KSRYTWCQSA-N); Podophyllotoxin o-benzylidene-beta-D-glucopyranoside (SBPPWJIDARICBS-QKFAOQDPSA-N); Polidocanol (ONJQDTZCDSESIW-UHFFFAOYSA-N); Poly(ri).poly(rc) (ACEVNMQDUCOKHT-UHFFFAOYSA-N); Polyestradiol phosphate (BBWXLCKRYRQQPL-ZBRFXRBCSA-N); Polymyxin b sulphate (HFMDLUQUEXNBOP-UHFFFAOYSA-N); Pomalidomide (UVSMNLNDYGZFPF-UHFFFAOYSA-N); Ponatinib (PHXJVRSECIGDHY-UHFFFAOYSA-N); Ponazuril (VBUNOIXRZNJNAD-UHFFFAOYSA-N); Porfiromycine (HRHKSTOGXBBQCB-UHFFFAOYSA-N); Posaconazole (RAGOYPUPXAKGKH-XAKZXMRKSA-N); Potassium dichromate (KMUONIBRACKNSN-UHFFFAOYSA-N); Poziotinib (LPFWVDIFUFFKJU-UHFFFAOYSA-N); Pracinostat (JHDKZFFAIZKUCU-ZRDIBKRKSA-N); Pralatrexate (OGSBUKJUDHAQEA-WMCAAGNKSA-N); Pralsetinib (GBLBJPZSROAGMF-SIYOEGHHSA-N); Pramoxine hydrochloride (SYCBXBCPLUFJID-UHFFFAOYSA-N); Pranlukast (NBQKINXMPLXUET-UHFFFAOYSA-N); Prasugrel (DTGLZDAWLRGWQN-UHFFFAOYSA-N); Prasugrel hydrochloride (JALHGCPDPSNJNY-UHFFFAOYSA-N); Pravastatin (TUZYXOIXSAXUGO-PZAWKZKUSA-N); Pravastatin sodium (VWBQYTRBTXKKOG-IYNICTALSA-M); Prazepam (MWQCHHACWWAQLJ-UHFFFAOYSA-N); Praziquantel (FSVJFNAIGNNGKK-UHFFFAOYSA-N); Prazosin (IENZQIKPVFGBNW-UHFFFAOYSA-N); Prazosin hydrochloride (WFXFYZULCQKPIP-UHFFFAOYSA-N); Prednisolone (OIGNJSKKLXVSLS-VWUMJDOOSA-N); Prednisolone tebutate (HUMXXHTVHHLNRO-KAJVQRHHSA-N); Prednisone (XOFYZVNMUHMLCC-ZPOLXVRWSA-N); Pregabalin (AYXYPKUFHZROOJ-ZETCQYMHSA-N); Prenylamine lactate (QPOFIDVRLWJICD-UHFFFAOYSA-N); Pretomanid (ZLHZLMOSPGACSZ-NSHDSACASA-N); Prexasertib (DOTGPNHGTYJDEP-UHFFFAOYSA-N); Prima-Imet (BGBNULCRKBVAKL-UHFFFAOYSA-N); Primaquine (INDBQLZJXZLFIT-UHFFFAOYSA-N); Primaquine phosphate (GJOHLWZHWQUKAU-UHFFFAOYSA-N); Primidone (DQMZLTXERSFNPB-UHFFFAOYSA-N); Primulic acid (LDPKDIVFHXKSCE-UHFFFAOYSA-N); Prinomastat (YKPYIPVDTNNYCN-INIZCTEOSA-N); Proadifen hydrochloride (FHIKZROVIDCMJA-UHFFFAOYSA-N); Probenecid (DBABZHXKTCFAPX-UHFFFAOYSA-N); Probucol (FYPMFJGVHOHGLL-UHFFFAOYSA-N); Procarbazine hydrochloride (DERJYEZSLHIUKF-UHFFFAOYSA-N); Prochlorperazine (WIKYUJGCLQQFNW-UHFFFAOYSA-N); Prochlorperazine edisylate (SWOUGRBFXFILIB-UHFFFAOYSA-N); Prochlorperazine maleate (DSKIOWHQLUWFLG-SPIKMXEPSA-N); Prodigiosin (SHUNBVWMKSXXOM-XDHOZWIPSA-N); Proflavine (WDVSHHCDHLJJJR-UHFFFAOYSA-N); Progesterone (RJKFOVLPORLFTN-LEKSSAKUSA-N); Proglumide (DGMKFQYCZXERLX-UHFFFAOYSA-N); Proguanil hydrochloride (SARMGXPVOFNNNG-UHFFFAOYSA-N); Promazine hydrochloride (JIVSXRLRGOICGA-UHFFFAOYSA-N); Promethazine (PWWVAXIEGOYWEE-UHFFFAOYSA-N); Propafenone (JWHAUXFOSRPERK-UHFFFAOYSA-N); Propafenone hydrochloride (XWIHRGFIPXWGEF-UHFFFAOYSA-N); Propamidine (WTFXJFJYEJZMFO-UHFFFAOYSA-N); Proparacaine hydrochloride (BFUUJUGQJUTPAF-UHFFFAOYSA-N); Propidium iodide (XJMOSONTPMZWPB-UHFFFAOYSA-M); Propofol (OLBCVFGFOZPWHH-UHFFFAOYSA-N); Propoxyphene hydrochloride (QMQBBUPJKANITL-MYXGOWFTSA-N); Propyl gallate (ZTHYODDOHIVTJV-UHFFFAOYSA-N); Propylparaben (QELSKZZBTMNZEB-UHFFFAOYSA-N); Propylthiouracil (KNAHARQHSZJURB-UHFFFAOYSA-N); Proscillaridin (MYEJFUXQJGHEQK-ALRJYLEOSA-N); Proscillaridin a (MYEJFUXQJGHEQK-OWBILYANSA-N); Protirelin (XNSAINXGIQZQOO-SRVKXCTJSA-N); Protoporphyrin ix (ZCFFYALKHPIRKJ-UHFFFAOYSA-N); Prucalopride (ZPMNHBXQOOVQJL-UHFFFAOYSA-N); Puromycin dihydrochloride (MKSVFGKWZLUTTO-FZFAUISWSA-N); Pyrazinamide (IPEHBUMCGVEMRF-UHFFFAOYSA-N); Pyrazineamine, sodium salt ion (ITIRENQGPSHVEU-UHFFFAOYSA-N) salts; Pyrazoloacridine (HZCWPKGYTCJSEB-UHFFFAOYSA-N); Pyrazoloacridine mesylate (OKVKOKPUEQTYKI-UHFFFAOYSA-N); Pyridin-3-yl 2-[[2-(trifluoromethyl)pyridin-4-yl]amino]benzoate (ODJBYXFDCRGEQH-UHFFFAOYSA-N); Pyrimethamine (WKSAUQYGYAYLPV-UHFFFAOYSA-N); Pyrithione (YBBJKCMMCRQZMA-UHFFFAOYSA-N); Pyrogallol (WQGWDDDVZFFDIG-UHFFFAOYSA-N); Pyronaridine (DJUFPMUQJKWIJB-UHFFFAOYSA-N); Pyroxamide (PTJGLFIIZFVFJV-UHFFFAOYSA-N); Pyrvinium pamoate (OOPDAHSJBRZRPH-UHFFFAOYSA-L); Quercetin (REFJWTPEDVJJIY-UHFFFAOYSA-N); Quercetin dihydrate (GMGIWEZSKCNYSW-UHFFFAOYSA-N); Quetiapine (URKOMYMAXPYINW-UHFFFAOYSA-N); Quinacrine (GPKJTRJOBQGKQK-UHFFFAOYSA-N); Quinacrine dihydrochloride (UDKVBVICMUEIKS-UHFFFAOYSA-N); Quinacrine dihydrochloride dihydrate (RZFNKJVCPDLQQA-UHFFFAOYSA-N); Quinestrol (PWZUUYSISTUNDW-VAFBSOEGSA-N); Quinidine (LOUPRKONTZGTKE-LHHVKLHASA-N); Quinine (LOUPRKONTZGTKE-WZBLMQSHSA-N); Quinine ethylcarbonate (NSBRKSWSLRQPJW-WWLNLUSPSA-N); Quinine sulfate dihydrate (ZHNFLHYOFXQIOW-LPYZJUEESA-N); Quinolinium, 1-ethyl-2-[(1-ethyl-2(1H)-quinolinylidene)methyl]-, chloride (MHQOURYMHSSLDM-UHFFFAOYSA-M); Quinolinium, 1-ethyl-2-[(1-ethyl-4(1H)-quinolinylidene)methyl]-, iodide (MYKATFBGLTVMQX-UHFFFAOYSA-M); Quinolinium,5-dimethyl-1-pentylpyrrol-3-yl)-vinyl]1,6-dimethyl-, chloride (ZKNVFCBYZIYGDP-UHFFFAOYSA-M); Quisinostat (PAWIYAYFNXQGAP-UHFFFAOYSA-N); Quizartinib (CVWXJKQAOSCOAB-UHFFFAOYSA-N); RAF265 (YABJJWZLRMPFSI-UHFFFAOYSA-N); RG7112 (QBGKPEROWUKSBK-QPPIDDCLSA-N); RGB-286638 free base (XLSYZSRXVVCHLS-UHFFFAOYSA-N); RH-1 (JKDLOGLNPDVUCX-UHFFFAOYSA-N); RO4929097 (OJPLJFIFUQPSJR-INIZCTEOSA-N); RO4987655 (FIMYFEGKMOCQKT-UHFFFAOYSA-N); ROR gama modulator 1 (LZLBRISQTJVZNP-UHFFFAOYSA-N); RQOHrs3jux (PXFDVONNVFXYEP-MCJVGQIASA-N); RWJ-67657 (QSUSKMBNZQHHPA-UHFFFAOYSA-N); Rabeprazole sodium (KRCQSTCYZUOBHN-UHFFFAOYSA-N); Rabusertib (SYYBDNPGDKKJDU-ZDUSSCGKSA-N); Racecadotril (ODUOJXZPIYUATO-UHFFFAOYSA-N); Racepinephrine hydrochloride (ATADHKWKHYVBTJ-UHFFFAOYSA-N); Radotinib (DUPWHXBITIZIKZ-UHFFFAOYSA-N); Rafoxanide (NEMNPWINWMHUMR-UHFFFAOYSA-N); Ralimetinib (XPPBBJCBDOEXDN-UHFFFAOYSA-N); Ralimetinib mesylate (NARMJPIBAXVUIE-UHFFFAOYSA-N); Raloxifene (GZUITABIAKMVPG-UHFFFAOYSA-N); Raloxifene hydrochloride (BKXVVCILCIUCLG-UHFFFAOYSA-N); Raltitrexed (IVTVGDXNLFLDRM-HNNXBMFYSA-N); Ramipril (HDACQVRGBOVJII-JBDAPHQKSA-N); Ramosetron hydrochloride (XIXYTCLDXQRHJO-RFVHGSKJSA-N); Ranitidine (VMXUWOKSQNHOCA-UKTHLTGXSA-N); Ranolazine (XKLMZUWKNUAPSZ-UHFFFAOYSA-N); Rapacuronium bromide (LVQTUXZKLGXYIU-GWSNJHLMSA-M); Ravoxertinib (RZUOCXOYPYGSKL-GOSISDBHSA-N); Razoxane (BMKDZUISNHGIBY-UHFFFAOYSA-N); Rebamipide (ALLWOAVDORUJLA-UHFFFAOYSA-N); Rebastinib (WVXNSAVVKYZVOE-UHFFFAOYSA-N); Reboxetine mesylate (CGTZMJIMMUNLQD-STYNFMPRSA-N); Refametinib (RDSACQWTXKSHJT-NSHDSACASA-N); Regorafenib (FNHKPVJBJVTLMP-UHFFFAOYSA-N); Remdesivir (RWWYLEGWBNMMLJ-YSOARWBDSA-N); Repaglinide (FAEKWTJYAYMJKF-QHCPKHFHSA-N); Replaced CAS registry number(s): 64256-11-7 (VJRAUFKOOPNFIQ-XAYJNONASA-N); Repotrectinib (FIKPXCOQUIZNHB-WDEREUQCSA-N); Rescinnamine (SZLZWPPUNLXJEA-QEGASFHISA-N); Reserpine (QEVHRUUCFGRFIF-MDEJGZGSSA-N); Resminostat (FECGNJPYVFEKOD-VMPITWQZSA-N); Resveratrol (LUKBXSAWLPMMSZ-OWOJBTEDSA-N); Retapamulin (STZYTFJPGGDRJD-FJJJPKKESA-N); Retigabine (PCOBBVZJEWWZFR-UHFFFAOYSA-N); Retinoid 9CUAB30 (PPGNMFUMZSAZCW-VOYUZAMQSA-N); Retinol (FPIPGXGPPPQFEQ-OVSJKPMPSA-N); Retinyl acetate (QGNJRVVDBSJHIZ-QHLGVNSISA-N); Rhodamine 123 (MYFATKRONKHHQL-UHFFFAOYSA-N); Ribavirin (IWUCXVSUMQZMFG-AFCXAGJDSA-N); Ribociclib (RHXHGRAEPCAFML-UHFFFAOYSA-N); Riboflavin tetrabutyrate (MJNIWUJSIGSWKK-BBANNHEPSA-N); Ricolinostat (QGZYDVAGYRLSKP-UHFFFAOYSA-N); Ridaforolimus (BUROJSBIWGDYCN-GAUTUEMISA-N); Rifampicin (JQXXHWHPUNPDRT-ZNQWNCHJSA-N); Rifampin (JQXXHWHPUNPDRT-KCFDLMDRSA-N); Rifamycin (HJYYPODYNSCCOU-ODRIEIDWSA-N); Rifapentine (WDZCUPBHRAEYDL-GZAUEHORSA-N); Rifaximin (NZCRJKRKKOLAOJ-XRCRFVBUSA-N); Rigosertib anion ion (OWBFCJROIKNMGD-BQYQJAHWSA-M) salts; Rigosertib sodium (VLQLUZFVFXYXQE-USRGLUTNSA-M); Rilmenidine (CQXADFVORZEARL-UHFFFAOYSA-N); Riluzole (FTALBRSUTCGOEG-UHFFFAOYSA-N); Rimiducid (GQLCLPLEEOUJQC-ZTQDTCGGSA-N); Rimonabant (JZCPYUJPEARBJL-UHFFFAOYSA-N); Risedronate sodium monohydrate (KLQNARDFMJRXSF-UHFFFAOYSA-M); Risperidone (RAPZEAPATHNIPO-UHFFFAOYSA-N); Rita (KZENBFUSKMWCJF-UHFFFAOYSA-N); Ritanserin (JUQLTPCYUFPYKE-UHFFFAOYSA-N); Ritonavir (NCDNCNXCDXHOMX-XGKFQTDJSA-N); Riviciclib (QLUYMIVVAYRECT-OCCSQVGLSA-N); Riviciclib (QLUYMIVVAYRECT-UHFFFAOYSA-N); Rizatriptan (ULFRLSNUDGIQQP-UHFFFAOYSA-N); Ro3280 (DJNZZLZKAXGMMC-UHFFFAOYSA-N); Rocaglamide (DAPAQENNNINUPW-IDAMAFBJSA-N); Rociletinib (HUFOZJXAKZVRNJ-UHFFFAOYSA-N); Rofecoxib (RZJQGNCSTQAWON-UHFFFAOYSA-N); Roflumilast (MNDBXUUTURYVHR-UHFFFAOYSA-N); Rogaratinib (HNLRRJSKGXOYNO-UHFFFAOYSA-N); Rolapitant (FIVSJYGQAIEMOC-ZGNKEGEESA-N); Rolipram (HJORMJIFDVBMOB-UHFFFAOYSA-N); Romidepsin (OHRURASPPZQGQM-QDBHDZETSA-N); Roniciclib (UELYDGOOJPRWGF-MFOHZAOFSA-N); Ropivacaine hydrochloride (NDNSIBYYUOEUSV-RSAXXLAASA-N); Roridin A,1OB-epoxy-(DYIWWIJYZCGJAG-KQQUZDAGSA-N); Rosiglitazone (YASAKCUCGLMORW-UHFFFAOYSA-N); Rotenone (JUVIOZPCNVVQFO-HBGVWJBISA-N); Rotigotine (KFQYTPMOWPVWEJ-INIZCTEOSA-N); Rubitecan (VHXNKPBCCMUMSW-FQEVSTJZSA-N); Ruboxistaurin (ZCBUQCWBWNUWSU-SFHVURJKSA-N); Rucaparib (HMABYWSNWIZPAG-UHFFFAOYSA-N); Rutaecarpine (ACVGWSKVRYFWRP-UHFFFAOYSA-N); Rutin (IKGXIBQEEMLURG-NVPNHPEKSA-N); Ruxolitinib (HFNKQEVNSGCOJV-OAHLLOKOSA-N); Rvp7C3xks6 (MQMHIOPMTAJOHV-SFTDATJTSA-N); S-(diethylarsino)-3-mercapto-1,2-propanediol (NTRDIXNRWQASAZ-UHFFFAOYSA-N); S-(diisopropylarsino)-3-mercapto-1,2-propanediol (BKAVMIAREOTDPR-UHFFFAOYSA-N); S-(dimethylarsino)-3-mercapto-1,2-propyl di-2-chloro-2-phenylacetate (SNSGULBHZHJFPY-UHFFFAOYSA-N); S-(dimethylarsino)-3-mercapto-1,2-propyl diacetate (WOCSEHQYFVWGTQ-UHFFFAOYSA-N); S-(dipropylarsino)-3-mercapto-1,2-propanediol (PITSAYCDYJYBJS-UHFFFAOYSA-N); S-Ethyl 2-cyano-6-methoxy-1(2H)-quinolinecarbothioate (RRBZCSKOQKCQJF-UHFFFAOYSA-N); S-Ruxolitinib (HFNKQEVNSGCOJV-HNNXBMFYSA-N); SAR125844 (ODIUNTQOXRXOIV-UHFFFAOYSA-N); SAR405838 (IDKAKZRYYDCJDU-AEPXTFJPSA-N); SB-505124 (WGZOTBUYUFBEPZ-UHFFFAOYSA-N); SB-590885 (MLSAQOINCGAULQ-QLTSDVKISA-N); SB-705498 (JYILLRHXRVTRSH-GFCCVEGCSA-N); SB-743921 (PGXYIBJJCLWJST-MUUNZHRXSA-N); SB590885 (MLSAQOINCGAULQ-QFMPWRQOSA-N); SC75741 (QNZVBFMXWNWVKG-UHFFFAOYSA-N); SCHEMBL11325880 (INEKMXPDNAXCDW-IHWYPQMZSA-N); SCHEMBL12143434 (OCQIQTVMFXIILG-UHFFFAOYSA-N); SCHEMBL12522967 (SGRBAVOESVMEMX-UHFFFAOYSA-N); SCHEMBL12756046 (LVHFSMVMWKBUOU-BWRTYORGSA-N); SCHEMBL13038047 (TZJALUIVHRYQQB-XGYHSCOOSA-N); SCHEMBL13910947 (BWJJTTMRISWTCO-XIYJGOOFSA-N); SCHEMBL15155348 (ISDIBDPXVWNENH-UHFFFAOYSA-N); SCHEMBL15588290 (TXNDCVCDINQHIX-OEXOOWIWSA-N); SCHEMBL16407012 (URRHSCSKLMBQLO-UHFFFAOYSA-N); SCHEMBL16526870 (AWDNKAGGWMOUMA-DTQAZKPQSA-N); SCHEMBL16769915 (KNXRNURZRRHJQF-FEZYOMQXSA-N); SCHEMBL16769927 (PUKFSPOXBOPJFM-ZZZAPRPPSA-N); SCHEMBL17406905 (RSOXZOFDCJMRMK-IOSLPCCCSA-N); SCHEMBL17712687 (LJJUNMWDURTMQG-SREVYHEPSA-N); SCHEMBL17712694 (ULDCRGDESYCVLB-SREVYHEPSA-N); SCHEMBL17845928 (VXDOHCKTCSQQRQ-UHFFFAOYSA-N); SCHEMBL17845972 (RXMMAYQOSOFMRN-UHFFFAOYSA-N); SCHEMBL17846035 (WEVXFHBMHZDTAQ-UHFFFAOYSA-N); SCHEMBL17858693 (UNTCRECTCLAQRP-UHFFFAOYSA-N); SCHEMBL17971367 (UIARLYUEJFELEN-IMDCKVJKSA-N); SCHEMBL18055016 (LILJYURXDAFOMS-XWPXHHJHSA-N); SCHEMBL18055021 (NOHZAMCDAHZSLN-OPHYPICJSA-N); SCHEMBL18055050 (DEMBWENYOAYGSY-XEXNZUFDSA-N); SCHEMBL18055187 (IUEMMLIYYOTWJV-UIOOHTPSSA-N); SCHEMBL18055188 (CDCLEXKRVZGCDZ-MRHPGVARSA-N); SCHEMBL18055202 (ZARDNQDTUPANLY-NWYFSMRYSA-N); SCHEMBL18055204 (HSJHYWHTQQRDKI-LLCNSABDSA-N); SCHEMBL18055214 (LNYFUFRCOQNPPN-AVJPYTMQSA-N); SCHEMBL18055294 (YWIMQDWEVLCLKX-KKAKTOJHSA-N); SCHEMBL18055319 (UVQLQSAWLGBINX-MYGWUIGISA-N); SCHEMBL18055326 (SROPLVGCYGSDSO-OPHYPICJSA-N); SCHEMBL18055327 (IRGDWFMYGQSWSP-PGYVEPDASA-N); SCHEMBL18055334 (KUQCTZDBHFNEGO-JYADXHFPSA-N); SCHEMBL18055371 (LOIGMHAJYDLWIT-OFTWBVLWSA-N); SCHEMBL18055372 (AOUVGNQKNIBYFZ-JYADXHFPSA-N); SCHEMBL18055373 (OIJRTDSHYYRZIA-PRHUCGOSSA-N); SCHEMBL18055376 (HBASMHUJPQYJFY-HTJBAMOESA-N); SCHEMBL18055406 (JUYOQLHUDZERPL-CLQIKQQLSA-N); SCHEMBL18055454 (RHQPUEZFTAFJBR-WZMKLFQESA-N); SCHEMBL18757995 (OKUXIUAKYDKHDY-UHFFFAOYSA-N); SCHEMBL18758234 (FZYSVVRVZAMZSG-UZJCWIEUSA-N); SCHEMBL18758328 (GHEACUPMEITBCD-IIWZZNCKSA-N); SCHEMBL1898193 (AOJJSUZBOXZQNB-BLSUDJMZSA-N); SCHEMBL19399322 (MGJBSCJJTZLOAQ-NZGBFQAGSA-N); SCHEMBL19399342 (AUKWSZDLKDIYOC-UIRHWMMOSA-N); SCHEMBL19399387 (QPJSTJCZNXRKLA-FFEKFOGKSA-N); SCHEMBL19399397 (VGVVHQXKYSPTSU-GJGHEGAFSA-N); SCHEMBL19399402 (PYXBWFFJLLDSNW-KHGVLYBHSA-N); SCHEMBL19399711 (BSTVUHBNWPTCHK-BZKNOHLFSA-N); SCHEMBL19399713 (GHFBWRGTHVTGIQ-AJNOBVHBSA-N); SCHEMBL19399724 (ZOWNCUQNSPIHFR-OXBNUMMXSA-N); SCHEMBL19399814 (OTUYFBBOIIBLOJ-GRWMVQBQSA-N); SCHEMBL19877264 (JVPCKCAKYWKMEG-UHFFFAOYSA-N); SCHEMBL20134582 (QMMMFDSIDJHQIT-RUJGCPEASA-N); SCHEMBL20574369 (KMTXOHJJHMCMLQ-ZHACJKMWSA-N); SCHEMBL2131861 (OFPUBDPPVJXQNW-PMERELPUSA-N); SCHEMBL21698625 (XAFUXTZQZONFAS-UHFFFAOYSA-N); SCHEMBL22070264 (RNTQVQUJOXMFAU-PMPSAXMXSA-N); SCHEMBL22085242 (GHUROFYDQUZIJT-CALCHBBNSA-N); SCHEMBL22085251 (QRZUINJXMUITFM-UHFFFAOYSA-N); SCHEMBL22085810 (WPMKFGCCABCSIX-UHFFFAOYSA-N); SCHEMBL22085812 (FWLCNEPGPUTNAP-UHFFFAOYSA-N); SCHEMBL22087313 (DVTRTLVTBRMZOD-UHFFFAOYSA-N); SCHEMBL22542570 (FMJOWOJKQACYPR-RUJGCPEASA-N); SCHEMBL22729177 (JEUMOXLCMVZJPH-UHFFFAOYSA-N); SCHEMBL22772360 (XAXJKDYUXBSJJT-UHFFFAOYSA-N); SCHEMBL22791189 (ZRDCIWCWMJWHDT-UHFFFAOYSA-N); SCHEMBL2280092 (VKFFLPNHOJIKMR-SANMLTNESA-N); SCHEMBL23046889 (JLAJVVQMCWAWNT-JTLKFFIHSA-N); SCHEMBL23252127 (YHTMBUNMHAWRKC-UHFFFAOYSA-N); SCHEMBL23343684 (XIWHTFJGIGNJFL-UHFFFAOYSA-N); SCHEMBL2346482 (BIODVRTUEIQALL-UHFFFAOYSA-N); SCHEMBL23525164 (ZBCWRXPKERICEK-QBBJDXIQSA-N); SCHEMBL23525172 (TZKRCEUZERTZRG-MFFBRLDPSA-N); SCHEMBL23525353 (SWJVFENQKSIPAW-WFEPKFSVSA-N); SCHEMBL23525406 (MVCSBMBMEVRKBJ-WVTPOXNGSA-N); SCHEMBL23525467 (LGICQGXAECCYRD-JXEFJUJFSA-N); SCHEMBL23551646 (COTPSIOVTKIEAC-YGFGYCEHSA-N); SCHEMBL23551647 (LXYTYIZKDUZUQR-XRZDBFDYSA-N); SCHEMBL23551718 (KPIARVYSTCFNSP-VCZAABBESA-N); SCHEMBL23551825 (WQNXUUZSYVGKJY-NRGLMKKFSA-N); SCHEMBL23551841 (GYTJKYKKCKXQAM-RWQNPXKYSA-N); SCHEMBL23551874 (JKIBXIQVJVECJX-QVHRZWFPSA-N); SCHEMBL23551893 (BAUNRFNHUXJPJD-ZFABGWHFSA-N); SCHEMBL23551958 (FJPDKSRWESDPSA-YBMWWVFYSA-N); SCHEMBL23551959 (BMMIPWCWFJSIIC-YZSBZJFESA-N); SCHEMBL23551961 (CQQHTMKQGHOCOH-GZZYFNPRSA-N); SCHEMBL23551979 (TUQGAGMNYHKQIG-OVPNZHTRSA-N); SCHEMBL23551980 (CFXXJUQVXWCZLZ-CRLCUMOYSA-N); SCHEMBL23551981 (WNGBPULEAZNVGN-OZPUQXOSSA-N); SCHEMBL23552022 (XXHGNNXHWZYAPM-DQJZIKQUSA-N); SCHEMBL23599644 (SRRTWLTZSXOGIU-UHFFFAOYSA-N); SCHEMBL23599651 (CWNVSUGMIQWYES-UHFFFAOYSA-N); SCHEMBL23599970 (VYSYDRUMWGZQKN-UHFFFAOYSA-N); SCHEMBL23599971 (WRMCNIXLWYMNMK-UHFFFAOYSA-N); SCHEMBL23600025 (FBMZPQGPKAUAPE-UHFFFAOYSA-N); SCHEMBL23600029 (IQYCVMBQFNOCOH-UHFFFAOYSA-N); SCHEMBL23600034 (RJMHEMLBDVDMHV-UHFFFAOYSA-N); SCHEMBL2618920 (GPDRVHRWVHPAOX-DEOSSOPVSA-N); SCHEMBL412603 (PMKMQVFQXFSHII-UHFFFAOYSA-N); SCHEMBL4548558 (JSNJYQNQNJMSSR-YZSQISJMSA-N); SCHEMBL5624264 (BMQWJFCUALXVCK-CQSZACIVSA-N); SCHEMBL62926 (VRQMAABPASPXMW-UHFFFAOYSA-N); SCHEMBL8465416 (UQXHMDDSVSOYHR-UHFFFAOYSA-N); SF2523 (BYTKNUOMWLJVNQ-UHFFFAOYSA-N); SGI-1776 (MHXGEROHKGDZGO-UHFFFAOYSA-N); SHetA2 (FWWKIYPMUZVMQG-UHFFFAOYSA-N); SNS-032 (OUSFTKFNBAZUKL-UHFFFAOYSA-N); SNS-314 Mesylate (FYCODPVDEFFWSR-UHFFFAOYSA-N); SNX-2112 (ZFVRYNYOPQZKDG-UHFFFAOYSA-N); SOP1812 (TZQVPFRBHPQZNM-UHFFFAOYSA-N); SR-05000001547 (QKRYGAZNLBVXHK-UHFFFAOYSA-N); SR-05000002056 (WDJUZGPOPHTGOT-KSVSUYAUSA-N); STK050748 (IPVCPNKDNPVCAK-UHFFFAOYSA-N); SU9516 (QNUKRWAIZMBVCU-WCIBSUBMSA-N); Sabutoclax (RAYNZUHYMMLQQA-ZEQRLZLVSA-N); Safinamide (NEMGRZFTLSKBAP-LBPRGKRZSA-N); Safingol (OTKJDMGTUTTYMP-ROUUACIJSA-N); Salarin c (KHGGKINOQOIBEV-PRBAJBALSA-N); Salicylanilide (WKEDVNSFRWHDNR-UHFFFAOYSA-N); Salinomycin (KQXDHUJYNAXLNZ-XQSDOZFQSA-N); Salinomycin sodium (YPZYGIQXBGHDBH-UZHRAPRISA-M); Salirasib (WUILNKCFCLNXOK-CFBAGHHKSA-N); Salmeterol (GIIZNNXWQWCKIB-UHFFFAOYSA-N); Salmeterol xinafoate (XTZNCVSCVHTPAI-UHFFFAOYSA-N); Samotolisib (ACCFLVVUVBJNGT-AWEZNQCLSA-N); Samuraciclib (YCVGLKWJKIKVBI-MJGOQNOKSA-N); Sancycline (MTCQOMXDZUULRV-ADOAZJKMSA-N); Sangivamycin hydrochloride (WTSGTUMQZSEZIW-UHFFFAOYSA-N); Sangivamycin, 6-aminothio-(HPCAEYRDTFITHI-UHFFFAOYSA-N); Sanguinarine ion (INVGWHRKADIJHF-UHFFFAOYSA-N) salts; Sanguinarine sulfate (VDZBWCCJLPCEQQ-UHFFFAOYSA-M); Sanguinarium chloride (GIZKAXHWLRYMLE-UHFFFAOYSA-M); Sapacitabine (LBGFKUUHOPIEMA-PEARBKPGSA-N); Sapanisertib (GYLDXIAOMVERTK-UHFFFAOYSA-N); Sapitinib (DFJSJLGUIXFDJP-UHFFFAOYSA-N); Saquinavir (QWAXKHKRTORLEM-UGJKXSETSA-N); Saquinavir mesylate (IRHXGOXEBNJUSN-YOXDLBRISA-N); Saracatinib (OUKYUETWWIPKQR-UHFFFAOYSA-N); Sarcolysin hydrochloride (OUUYBRCCFUEMLH-UHFFFAOYSA-N); Saridegib (HZLFFNCLTRVYJG-WWGOJCOQSA-N); Sarmustine (HYHJFNXFVPGMBI-UHFFFAOYSA-N); Sarpogrelate (FFYNAVGJSYHHFO-UHFFFAOYSA-N); Sarpogrelate hydrochloride (POQBIDFFYCYHOB-UHFFFAOYSA-N); Scillirosidin, glycoside (SOJXCEWHLPYBAF-UHFFFAOYSA-N); Scopoletin (RODXRVNMMDRFIK-UHFFFAOYSA-N); Sdz htf 919 (IKBKZGMPCYNSLU-JAIQZWGSSA-N); Seclidemstat (MVSQDUZRRVBYLA-HYARGMPZSA-N); Secoisolariciresinol (PUETUDUXMCLALY-HOTGVXAUSA-N); Securinine (SWZMSZQQJRKFBP-WZRBSPASSA-N); Selamectin (AFJYYKSVHJGXSN-XHKIUTQPSA-N); Seliciclib (BTIHMVBBUGXLCJ-OAHLLOKOSA-N); Selinexor (DEVSOMFAQLZNKR-RJRFIUFISA-N); Selitrectinib (OEBIHOVSAMBXIB-SJKOYZFVSA-N); Selonsertib (YIDDLAAKOYYGJG-UHFFFAOYSA-N); Selumetinib (CYOHGALHFOKKQC-UHFFFAOYSA-N); Semagacestat (PKXWXXPNHIWQHW-RCBQFDQVSA-N); Semapimod (PWDYHMBTPGXCSN-VCBMUGGBSA-N); Semaxanib (WUWDLXZGHZSWQZ-WQLSENKSSA-N); Semustine (FVLVBPDQNARYJU-UHFFFAOYSA-N); Senexin b (VNADJTWHOAMTLY-UHFFFAOYSA-N); Sensit (NJXWZWXCHBNOOG-UHFFFAOYSA-N); Seocalcitol (LVLLALCJVJNGQQ-SEODYNFXSA-N); Sepantronium bromide (QBIYUDDJPRGKNJ-UHFFFAOYSA-M); Sepantronium ion (OTSOOHRUMBRSHZ-UHFFFAOYSA-N) salts; Septacidin (YBZRLMLGUBIIDN-UHFFFAOYSA-N); Serabelisib (BLGWHBSBBJNKJO-UHFFFAOYSA-N); Seratrodast (ZBVKEHDGYSLCCC-UHFFFAOYSA-N); Serdemetan (CEGSUKYESLWKJP-UHFFFAOYSA-N); Serotonin (QZAYGJVTTNCVMB-UHFFFAOYSA-N); Sertaconazole nitrate (HAAITRDZHUANGT-UHFFFAOYSA-N); Sertindole (GZKLJWGUPQBVJQ-UHFFFAOYSA-N); Sertraline (VGKDLMBJGBXTGI-SJCJKPOMSA-N); Sertraline hydrochloride (BLFQGGGGFNSJKA-XHXSRVRCSA-N); Sesbanimide (ULQATHQJWVNXEJ-UHFFFAOYSA-N); Setoglaucine (GRPFBMKYXAYEJM-UHFFFAOYSA-M); Seviteronel (ZBRAJOQFSNYJMF-SFHVURJKSA-N); Sgx-523 (BCZUAADEACICHN-UHFFFAOYSA-N); Sibutramine (UNAANXDKBXWMLN-UHFFFAOYSA-N); Silatecan (XUSKJHCMMWAAHV-SANMLTNESA-N); Sildenafil (BNRNXUUZRGQAQC-UHFFFAOYSA-N); Sildenafil citrate (DEIYFTQMQPDXOT-UHFFFAOYSA-N); Silibinin (SEBFKMXJBCUCAI-HKTJVKLFSA-N); Silmitasertib (MUOKSQABCJCOPU-UHFFFAOYSA-N); Silver benzoate, 99% (GQCYXEOBZQXHPO-UHFFFAOYSA-N); Silver methylsulfonate (GTBQRHOYAUGRPV-UHFFFAOYSA-N); Simalikalactone d (OKIKYYZNNZCZRX-QIMXXMOASA-N); Simurosertib (XGVXKJKTISMIOW-ZDUSSCGKSA-N); Simvastatin (RYMZZMVNJRMUDD-HGQWONQESA-N); Sinensetin (LKMNXYDUQXAUCZ-UHFFFAOYSA-N); Sinomenine (INYYVPJSBIVGPH-QHRIQVFBSA-N); Sirolimus (QFJCIRLUMZQUOT-HPLJOQBZSA-N); Sitagliptin (MFFMDFFZMYYVKS-SECBINFHSA-N); Sitaxentan (PHWXUGHIIBDVKD-UHFFFAOYSA-N); Sitravatinib (WLAVZAAODLTUSW-UHFFFAOYSA-N); Sjg-136 (RWZVMMQNDHPRQD-SFTDATJTSA-N); Sns-314 (FAYAUAZLLLJJGH-UHFFFAOYSA-N); Snx-5422 (AVDSOVJPJZVBTC-UHFFFAOYSA-N); Sodium azotomycin ion (ZPTLVAFRSYVZBM-UHFFFAOYSA-P) salts; Sodium chloride (FAPWRFPIFSIZLT-UHFFFAOYSA-M); Sodium dichloroacetate (LUPNKHXLFSSUGS-UHFFFAOYSA-M); Sodium; 2-[(2-phosphonoacetyl)amino]butanedioic acid ion (NTPHVSVNFQQSAL-UHFFFAOYSA-N) salts; Sofalcone (GFWRVVCDTLRWPK-KPKJPENVSA-N); Sofosbuvir (TTZHDVOVKQGIBA-IQWMDFIBSA-N); Solifenacin hydrochloride (YAUBKMSXTZQZEB-VROPFNGYSA-N); Sonidegib (VZZJRYRQSPEMTK-CALCHBBNSA-N); Sonolisib (QIUASFSNWYMDFS-NILGECQDSA-N); Sorafenib (MLDQJTXFUGDVEO-UHFFFAOYSA-N); Sorafenib tosylate (IVDHYUQIDRJSTI-UHFFFAOYSA-N); Sotalol (ZBMZVLHSJCTVON-UHFFFAOYSA-N); Sotorasib (NXQKSXLFSAEQCZ-SFHVURJKSA-N); Sotrastaurin (OAVGBZOFDPFGPJ-UHFFFAOYSA-N); Spebrutinib (KXBDTLQSDKGAEB-UHFFFAOYSA-N); Spectinomycin (UNFWWIHTNXNPBV-WXKVUWSESA-N); Spermine (PFNFFQXMRSDOHW-UHFFFAOYSA-N); Spiromustine (QNKJFXARIMSDBR-UHFFFAOYSA-N); Spironolactone (LXMSZDCAJNLERA-ZHYRCANASA-N); Squalene (YYGNTYWPHWGJRM-AAJYLUCBSA-N); Stains-all (MPBRYMWMMKKRGC-UHFFFAOYSA-M); Stannane,4,5-trichlorophenoxy)-ion (VSMHISQJZOTNJC-UHFFFAOYSA-M) salts; Stanozolol (LKAJKIOFIWVMDJ-IYRCEVNGSA-N); Staurosporine (HKSZLNNOFSGOKW-FYTWVXJKSA-N); Stavudine (XNKLLVCARDGLGL-JGVFFNPUSA-N); Stilbazium iodide (BYIRBDUHSVOFLU-UHFFFAOYSA-M); Stiripentol (IBLNKMRFIPWSOY-FNORWQNLSA-N); Streptonigrin (PVYJZLYGTZKPJE-UHFFFAOYSA-N); Streptovitacin a (NFDQYBUVHVRNNY-UHFFFAOYSA-N); Streptozocin (ZSJLQEPLLKMAKR-GKHCUFPYSA-N); Strophanthin (FHIREUBIEIPPMC-UHFFFAOYSA-N); Subasumstat (LXRZVMYMQHNYJB-UNXOBOICSA-N); Substance p (ADNPLDHMAVUMIW-CUZNLEPHSA-N); Succinic acid (KDYFGRWQOYBRFD-UHFFFAOYSA-N); Succinobucol (RKSMVPNZHBRNNS-UHFFFAOYSA-N); Sufentanil citrate (OJCZPLDERGDQRJ-UHFFFAOYSA-N); Sulbactam (FKENQMMABCRJMK-RITPCOANSA-N); Sulconazole nitrate (CRKGMGQUHDNAPB-UHFFFAOYSA-N); Sulfachlorpyridazine (XOXHILFPRYWFOD-UHFFFAOYSA-N); Sulfadiazine (SEEPANYCNGTZFQ-UHFFFAOYSA-N); Sulfadimethoxine (ZZORFUFYDOWNEF-UHFFFAOYSA-N); Sulfadoxine (PJSFRIWCGOHTNF-UHFFFAOYSA-N); Sulfameter (GPTONYMQFTZPKC-UHFFFAOYSA-N); Sulfamethizole (VACCAVUAMIDAGB-UHFFFAOYSA-N); Sulfamethoxazole (JLKIGFTWXXRPMT-UHFFFAOYSA-N); Sulfanilamide (FDDDEECHVMSUSB-UHFFFAOYSA-N); Sulfaquinoxaline (NHZLNPMOSADWGC-UHFFFAOYSA-N); Sulfatinib (TTZSNFLLYPYKIL-UHFFFAOYSA-N); Sulfiram (CTPKSRZFJSJGML-UHFFFAOYSA-N); Sulforaphane (SUVMJBTUFCVSAD-UHFFFAOYSA-N); Sulindac (MLKXDPUZXIRXEP-MFOYZWKCSA-N); Suloctidil (BFCDFTHTSVTWOG-UHFFFAOYSA-N); Sulpiride (BGRJTUBHPOOWDU-UHFFFAOYSA-N); Sun 2071 (JNBOCJQZCHEASA-XNTDXEJSSA-N); Sunitinib (WINHZLLDWRZWRT-ATVHPVEESA-N); Sunitinib malate (LBWFXVZLPYTWQI-IPOVEDGCSA-N); Supinoxin (KAKPGJJRYRYSTP-UHFFFAOYSA-N); Suramin (FIAFUQMPZJWCLV-UHFFFAOYSA-N); Symetamine (FZDQLVGKBSJVPZ-UHFFFAOYSA-N); T-2 mycotoxin (BXFOFFBJRFZBQZ-PPFYZKLNSA-N); TAK-243 (KJDAGXLMHXUAGV-DGWLBADLSA-N); TAK-632 (OJFKUJDRGJSAQB-UHFFFAOYSA-N); TAK-733 (RCLQNICOARASSR-SECBINFHSA-N); TAK-901 (WKDACQVEJIVHMZ-UHFFFAOYSA-N); TAK-960 (GWRSATNRNFYMDI-UHFFFAOYSA-N); TAS-115 (ORRNXRYWGDUDOG-UHFFFAOYSA-N); TAS-116 (NVVPMZUGELHVMH-UHFFFAOYSA-N); TAS6417 (MKCYPWYURWOKST-INIZCTEOSA-N); TCMDC-138079 (YDEWUVKTIHHCKK-UHFFFAOYSA-N); TCS JNK 5a (WQGDQGAFSDMBLA-UHFFFAOYSA-N); TG101209 (JVDOKQYTTYUYDV-UHFFFAOYSA-N); TGX-221 (CPRAGQJXBLMUEL-UHFFFAOYSA-N); THP-adriamycin HCl (ZPHYPKKFSHAVOE-XFQXKDBGSA-N); THZ1 (OBJNFLYHUXWUPF-IZZDOVSWSA-N); THZ531 (RUBYHLPRZRMTJO-MOVYNIQHSA-N); TP-3654 (XRNVABDYQLHODA-UHFFFAOYSA-N); Tacedinaline (VAZAPHZUAVEOMC-UHFFFAOYSA-N); Tacrine (YLJREFDVOIBQDA-UHFFFAOYSA-N); Tacrolimus (QJJXYPPXXYFBGM-LFZNUXCKSA-N); Tafetinib (KGSRYTUWXUESJK-FXBPSFAMSA-N); Tak-285 (ZYQXEVJIFYIBHZ-UHFFFAOYSA-N); Tak-593 (DZFZXPPHBWCXPQ-UHFFFAOYSA-N); Tak-715 (HEKAIDKUDLCBRU-UHFFFAOYSA-N); Taladegib (SZBGQDXLNMELTB-UHFFFAOYSA-N); Talaporfin (SIEXFRDYNDREBM-BRXYURPTSA-N); Talarozole (SNFYYXUGUBUECJ-UHFFFAOYSA-N); Talazoparib (HWGQMRYQVZSGDQ-HZPDHXFCSA-N); Taletrectinib (free base) (HEVHTYMYEMEBPX-HZPDHXFCSA-N); Talinolol (MXFWWQICDIZSOA-UHFFFAOYSA-N); Talmapimod (ZMELOYOKMZBMRB-DLBZAZTESA-N); Tamatinib (NHHQJBCNYHBUSI-UHFFFAOYSA-N); Tame (FKMJXALNHKIDOD-LBPRGKRZSA-N); Tamibarotene (MUTNCGKQJGXKEM-UHFFFAOYSA-N); Tamoxifen (NKANXQFJJICGDU-QPLCGJKRSA-N); Tamoxifen citrate (FQZYTYWMLGAPFJ-OQKDUQJOSA-N); Tamsulosin hydrochloride (ZZIZZTHXZRDOFM-XFULWGLBSA-N); Tandospirone (CEIJFEGBUDEYSX-FZDBZEDMSA-N); Tandutinib (UXXQOJXBIDBUAC-UHFFFAOYSA-N); Tanespimycin (AYUNIORJHRXIBJ-TXHRRWQRSA-N); Tangeretin (ULSUXBXHSYSGDT-UHFFFAOYSA-N); Tanshinone i (AIGAZQPHXLWMOJ-UHFFFAOYSA-N); Tanshinone iia (HYXITZLLTYIPOF-UHFFFAOYSA-N); Tapotoclax (JQNINBDKGLWYMU-GEAQBIRJSA-N); Taranabant (QLYKJCMUNUWAGO-GAJHUEQPSA-N); Tariquidar (LGGHDPFKSSRQNS-UHFFFAOYSA-N); Taselisib (BEUQXVWXFDOSAQ-UHFFFAOYSA-N); Tazarotene (OGQICQVSFDPSEI-UHFFFAOYSA-N); Tazemetostat (NSQSAUGJQHDYNO-UHFFFAOYSA-N); Tecalcet (ZVQUCWXZCKWZBP-CQSZACIVSA-N); Tedizolid (XFALPSLJIHVRKE-GFCCVEGCSA-N); Tedizolid phosphate (QCGUSIANLFXSGE-GFCCVEGCSA-N); Tegafur (WFWLQNSHRPWKFK-UHFFFAOYSA-N); Tegaserod (IKBKZGMPCYNSLU-RGVLZGJSSA-N); Tegaserod maleate (CPDDZSSEAVLMRY-FEQFWAPWSA-N); Tegavivint (OMWCXCBGEFHCTN-FGYAAKKASA-N); Teglarinad ion (RXXJCNMVVRGOJB-UHFFFAOYSA-O) salts; Telaglenastat (PRAAPINBUWJLGA-UHFFFAOYSA-N); Telaprevir (BBAWEDCPNXPBQM-GDEBMMAJSA-N); Telatinib (QFCXANHHBCGMAS-UHFFFAOYSA-N); Telcagepant (CGDZXLJGHVKVIE-DNVCBOLYSA-N); Telithromycin (LJVAJPDWBABPEJ-PNUFFHFMSA-N); Telmisartan (RMMXLENWKUUMAY-UHFFFAOYSA-N); Telotristat ethyl (MDSQOJYHHZBZKA-GBXCKJPGSA-N); Telotristat etiprate (XSFPZBUIBYMVEA-CELUQASASA-N); Temocapril hydrochloride (XDDQNOKKZKHBIX-ASBZXGSUSA-N); Temoporfin (LYPFDBRUNKHDGX-UHFFFAOYSA-N); Temozolomide (BPEGJWRSRHCHSN-UHFFFAOYSA-N); Temsirolimus (CBPNZQVSJQDFBE-FUXHJELOSA-N); Temuterkib (JNPRPMBJODOFEC-UHFFFAOYSA-N); Tenatoprazole (ZBFDAUIVDSSISP-UHFFFAOYSA-N); Teniposide (NRUKOCRGYNPUPR-QBPJDGROSA-N); Teniposide [usan](NRUKOCRGYNPUPR-OQMCATNJSA-N); Tenofovir (SGOIRFVFHAKUTI-ZCFIWIBFSA-N); Tenofovir disoproxil fumarate (VCMJCVGFSROFHV-WZGZYPNHSA-N); Tenoxicam (LZNWYQJJBLGYLT-UHFFFAOYSA-N); Tepotinib (AHYMHWXQRWRBKT-UHFFFAOYSA-N); Tepoxalin (XYKWNRUXCOIMFZ-UHFFFAOYSA-N); Terameprocol (ORQFDHFZSMXRLM-IYBDPMFKSA-N); Terbinafine (DOMXUEMWDBAQBQ-WEVVVXLNSA-N); Terconazole (BLSQLHNBWJLIBQ-OZXSUGGESA-N); Terephthalanilide,4″-bis(2-imidazolin-2-yl)-, dihydrochloride (UERKHENLJAOVFL-UHFFFAOYSA-N); Terephthalanilide,4″-bis(2-imidazolin-2-ylamino)-, dihydrochloride (IJOSETYOWUSQTH-UHFFFAOYSA-N); Terephthalanilide,4″-di-2-imidazolin-2-yl-, dihydrochloride (WOODYQJVORPYOD-UHFFFAOYSA-N); Terfenadine (GUGOEEXESWIERI-UHFFFAOYSA-N); Terguride (JOAHPSVPXZTVEP-YXJHDRRASA-N); Teriflunomide (UTNUDOFZCWSZMS-YFHOEESVSA-N); Terodiline hydrochloride (RNGHAJVBYQPLAZ-UHFFFAOYSA-N); Tesevatinib (HVXKQKFEHMGHSL-GOOCMWNKSA-N); Tesmilifene (NFIXBCVWIPOYCD-UHFFFAOYSA-N); Testosterone (MUMGGOZAMZWBJJ-DYKIIFRCSA-N); Testosterone cypionate (HPFVBGJFAYZEBE-ZLQWOROUSA-N); Testosterone decanoate (LBERVHLCXUMDOT-MPZZESAYSA-N); Testosterone isocaproate (PPYHLSBUTAPNGT-BKWLFHPQSA-N); Testosterone phenylpropionate (HHSXYDOROIURIP-FEZCWRLCSA-N); Testosterone propionate (PDMMFKSKQVNJMI-BLQWBTBKSA-N); Tetracaine (GKCBAIGFKIBETG-UHFFFAOYSA-N); Tetracaine hydrochloride (PPWHTZKZQNXVAE-UHFFFAOYSA-N); Tetracycline hydrochloride (YCIHPQHVWDULOY-FMZCEJRJSA-N); Tetraethylammonium chloride (YMBCJWGVCUEGHA-UHFFFAOYSA-M); Tetraethyleniminobenzo-quinone (GNSPPAGSLFPHSZ-UHFFFAOYSA-N); Tetrahydropalmatine (AEQDJSLRWYMAQI-KRWDZBQOSA-N); Tetrahydrozoline hydrochloride (BJORNXNYWNIWEY-UHFFFAOYSA-N); Tetramethylthiuram monosulfide (REQPQFUJGGOFQL-UHFFFAOYSA-N); Tetrandrine (WVTKBKWTSCPRNU-KYJUHHDHSA-N); Tetrocarcin a, sodium salt (QOPRNPHPHOIJNY-NUIAMOMSSA-N); Thalidomide (UEJJHQNACJXSKW-UHFFFAOYSA-N); Thenalidine (KLOHYVOVXOUKQI-UHFFFAOYSA-N); Thenyldiamine (RCGYDFVCAAKKNG-UHFFFAOYSA-N); Theobromine (YAPQBXQYLJRXSA-UHFFFAOYSA-N); Theophylline (ZFXYFBGIUFBOJW-UHFFFAOYSA-N); Thiabendazole (WJCNZQLZVWNLKY-UHFFFAOYSA-N); Thiactin (NSFFHOGKXHRQEW-BXVAPQLOSA-N); Thiamphenicol (OTVAEFIXJLOWRX-NXEZZACHSA-N); Thiethylperazine malate (GTHHLZDYRHLACN-UHFFFAOYSA-N); Thimerosal (RTKIYNMVFMVABJ-UHFFFAOYSA-L); Thioacetazone (SRVJKTDHMYAMHA-WUXMJOGZSA-N); Thiocolchicine (CMEGANPVAXDBPL-INIZCTEOSA-N); Thioctic acid (AGBQKNBQESQNJD-UHFFFAOYSA-N); Thioguanine (WYWHKKSPHMUBEB-UHFFFAOYSA-N); Thiolutin (MHMRAFONCSQAIA-UHFFFAOYSA-N); Thioridazine (KLBQZWRITKRQQV-UHFFFAOYSA-N); Thioridazine hydrochloride (NZFNXWQNBYZDAQ-UHFFFAOYSA-N); Thiosangivamycin (XZOKDXWQSCIKCM-UHFFFAOYSA-N); Thiosemicarbazone h (LYIUREYWAKPINL-FOWTUZBSSA-N); Thiotepa (FOCVUCIESVLUNU-UHFFFAOYSA-N); Thiram (KUAZQDVKQLNFPE-UHFFFAOYSA-N); Thonzonium bromide (WBWDWFZTSDZAIG-UHFFFAOYSA-M); Thymine (RWQNBRDOKXIBIV-UHFFFAOYSA-N); Tiamulin (UURAUHCOJAIIRQ-QGLSALSOSA-N); Tibolone (WZDGZWOAQTVYBX-XOINTXKNSA-N); Tic mustard (XWRSGLHADOYUFN-PFONDFGASA-N); Ticlopidine (PHWBOXQYWZNQIN-UHFFFAOYSA-N); Tideglusib (PMJIHLSCWIDGMD-UHFFFAOYSA-N); Tilarginine (NTNWOCRCBQPEKQ-YFKPBYRVSA-N); Tilmicosin (JTSDBFGMPLKDCD-XVFHVFLVSA-N); Timonacic (DZLNHFMRPBPULJ-UHFFFAOYSA-N); Tin organometallic (DOICFEXUJKISKP-UHFFFAOYSA-L); Tinoridine (PFENFDGYVLAFBR-UHFFFAOYSA-N); Tinostamustine (GISXTRIGVCKQBX-UHFFFAOYSA-N); Tioconazole (QXHHHPZILQDDPS-UHFFFAOYSA-N); Tiopronin (YTGJWQPHMWSCST-UHFFFAOYSA-N); Tiotixene (GFBKORZTTCHDGY-UWVJOHFNSA-N); Tioxidazole (HLLICFJUWSZHRJ-UHFFFAOYSA-N); Tioxolone (SLYPOVJCSQHITR-UHFFFAOYSA-N); Tipifarnib (PLHJCIYEEKOWNM-HHHXNRCGSA-N); Tiplasinin (ODXQFEWQSHNQNI-UHFFFAOYSA-N); Tirapazamine (ORYDPOVDJJZGHQ-UHFFFAOYSA-N); Tirofiban hydrochloride (HWAAPJPFZPHHBC-FGJQBABTSA-N); Tivantinib (UCEQXRCJXIVODC-PMACEKPBSA-N); Tivozanib (SPMVMDHWKHCIDT-UHFFFAOYSA-N); Tizanidine (XFYDIVBRZNQMJC-UHFFFAOYSA-N); Tizoxanide (FDTZUTSGGSRHQF-UHFFFAOYSA-N); Tolcapone (MIQPIUSUKVNLNT-UHFFFAOYSA-N); Tolfenamic acid (YEZNLOUZAIOMLT-UHFFFAOYSA-N); Tolnaftate (FUSNMLFNXJSCDI-UHFFFAOYSA-N); Tolonium chloride (GEDVVYWLPUPJJZ-UHFFFAOYSA-N); Tolperisone hydrochloride (ZBUVYROEHQQAKL-UHFFFAOYSA-N); Tolterodine (OOGJQPCLVADCPB-HXUWFJFHSA-N); Tolvaptan (GYHCTFXIZSNGJT-UHFFFAOYSA-N); Tomaymycin (UQVNRKBFAXNOGA-HRZZMDIGSA-N); Tomivosertib (HKTBYUWLRDZAJK-UHFFFAOYSA-N); Topiramate (KJADKKWYZYXHBB-XBWDGYHZSA-N); Topotecan (UCFGDBYHRUNTLO-QHCPKHFHSA-N); Topotecan hydrochloride (DGHHQBMTXTWTJV-BQAIUKQQSA-N); Torcetrapib (CMSGWTNRGKRWGS-NQIIRXRSSA-N); Toremifene (XFCLJVABOIYOMF-QPLCGJKRSA-N); Toremifene citrate (IWEQQRMGNVVKQW-OQKDUQJOSA-N); Torin 2 (GUXXEUUYCAYESJ-UHFFFAOYSA-N); Torisel (tm) (CBPNZQVSJQDFBE-VBSVFFBFSA-N); Tosedostat (FWFGIHPGRQZWIW-SQNIBIBYSA-N); Toxin HT 2 (PNKLMTPXERFKEN-ZIOSACBISA-N); Tozasertib (GCIKSSRWRFVXBI-UHFFFAOYSA-N); Trabectedin (PKVRCIRHQMSYJX-AIFWHQITSA-N); Trametinib (LIRYPHYGHXZJBZ-UHFFFAOYSA-N); Tranexamic acid (GYDJEQRTZSCIOI-UHFFFAOYSA-N); Tranilast (NZHGWWWHIYHZNX-CSKARUKUSA-N); Trapoxin b (LLOKIGWPNVSDGJ-AFBVCZJXSA-N); Travoprost (MKPLKVHSHYCHOC-AHTXBMBWSA-N); Trenbolone acetate (CMRJPMODSSEAPL-FYQPLNBISA-N); Tresulfan (YCPOZVAOBBQLRI-UHFFFAOYSA-N); Tretazicar (WOCXQMCIOTUMJV-UHFFFAOYSA-N); Tretinoin (SHGAZHPCJJPHSC-YCNIQYBTSA-N); Triacetylresveratrol (PDAYUJSOJIMKIS-SNAWJCMRSA-N); Triamcinolone (GFNANZIMVAIWHM-OBYCQNJPSA-N); Triamterene (FNYLWPVRPXGIIP-UHFFFAOYSA-N); Triapine (XMYKNCNAZKMVQN-NYYWCZLTSA-N); Triazinate (HKPVIFTWECXNPY-UHFFFAOYSA-N); Tribromsalan (KVSKGMLNBAPGKH-UHFFFAOYSA-N); Tributyl((methoxy(phenyl)acetyl)oxy)stannane (UJNPNWZXIJKMGJ-UHFFFAOYSA-M); Tributyl(stearoyloxy)stannane (GQZXITZWLBSSOI-UHFFFAOYSA-M); Tributyl(undec-10-enoyloxy)stannane (XEMFLPGDKXELSO-UHFFFAOYSA-M); Tributyllead chloride (OCQHDJVUUKFOFH-UHFFFAOYSA-M); Tributylstannyl 2-acetoxy-2-phenyl-acetate (WJLNJZMTFIOXKN-UHFFFAOYSA-M); Tributyltin 2,4-dichlorobenzoate (FECJFQGVPGQQCF-UHFFFAOYSA-M); Tributyltin laurate (HFFZSMFXOBHQLV-UHFFFAOYSA-M); Tributyltin propionate (AHIXTTGLRFPWJF-UHFFFAOYSA-M); Trichostatin a (RTKIYFITIVXBLE-QEQCGCAPSA-N); Trichothec-9-en-4-ol,13-epoxy-, acetate, (4beta)-(HNEGCRMUYSKRRR-OKZUVXGGSA-N); Triciribine (HOGVTUZUJGHKPL-HTVVRFAVSA-N); Triciribine phosphate (URLYINUFLXOMHP-HTVVRFAVSA-N); Triclabendazole (NQPDXQQQCQDHHW-UHFFFAOYSA-N); Triclocarban (ICUTUKXCWQYESQ-UHFFFAOYSA-N); Triclosan (XEFQLINVKFYRCS-UHFFFAOYSA-N); Tricyclo[3.3.1.1-3,7-]tetraarsoxane (KTTMEOWBIWLMSE-UHFFFAOYSA-N); Triethylenemelamine (IUCJMVBFZDHPDX-UHFFFAOYSA-N); Triferric doxorubicin (AOJJSUZBOXZQNB-UHFFFAOYSA-N); Trifluoperazine (ZEWQUBUPAILYHI-UHFFFAOYSA-N); Trifluoperazine hydrochloride (BXDAOUXDMHXPDI-UHFFFAOYSA-N); Triflupromazine (XSCGXQMFQXDFCW-UHFFFAOYSA-N); Triflupromazine hydrochloride (FTNWXGFYRHWUKG-UHFFFAOYSA-N); Trifluridine (VSQQQLOSPVPRAZ-RRKCRQDMSA-N); Triglycidyl isocyanurate (OUPZKGBUJRBPGC-UHFFFAOYSA-N); Trihexyltetradecylphosphonium bis(trifluoromethylsulfonyl)amide (HYNYWFRJHNNLJA-UHFFFAOYSA-N); Trilaciclib (PDGKHKMBHVFCMG-UHFFFAOYSA-N); Trilostane (KVJXBPDAXMEYOA-CXANFOAXSA-N); Trimethaphan camsylate (HALWUDBBYKMYPW-UHFFFAOYSA-M); Trimetrexate (NOYPYLRCIDNJJB-UHFFFAOYSA-N); Trimetrexate glucuronic acid salt (ZCJXQWYMBJYJNB-UHFFFAOYSA-N); Trimipramine maleate (YDGHCKHAXOUQOS-BTJKTKAUSA-N); Triostin a (GULVULFEAVZHHC-UHFFFAOYSA-N); Trioxsalen (FMHHVULEAZTJMA-UHFFFAOYSA-N); Triparanol (SYHDSBBKRLVLFF-UHFFFAOYSA-N); Tripelennamine citrate (GGRBYIUPUOYRLQ-UHFFFAOYSA-N); Triphenyltin chloride (NJVOZLGKTAPUTQ-UHFFFAOYSA-M); Triprolidine (CBEQULMOCCWAQT-WOJGMQOQSA-N); Triptil (OGQDIIKRQRZXJH-UHFFFAOYSA-N); Triptolide (DFBIRQPKNDILPW-CIVMWXNOSA-N); Triptonide (SWOVVKGLGOOUKI-ZHGGVEMFSA-N); Tris(4-tert-butyl-2,6-dimethoxyphenyl)methanol (ZFRIXUZTJBWSAG-UHFFFAOYSA-N); Tritylcysteine (DLMYFMLKORXJPO-UHFFFAOYSA-N); Trofosfamide (UMKFEPPTGMDVMI-UHFFFAOYSA-N); Troglitazone (GXPHKUHSUJUWKP-UHFFFAOYSA-N); Tromethamine (LENZDBCJOHFCAS-UHFFFAOYSA-N); Tropisetron (ZNRGQMMCGHDTEI-FUNVUKJBSA-N); Trospium ion (OYYDSUSKLWTMMQ-AIZNXBIQSA-N) salts; Trovafloxacin mesylate (DYNZICQDCVYXFW-GIPYJWDTSA-N); Trypaflavine hydrochloride (OEGQPXPIKZELFM-UHFFFAOYSA-N); Trypan blue (GLNADSQYFUSGOU-UHFFFAOYSA-J); Tubulosine (JRVWIILYWSBUMC-PRUVNFMMSA-N); Tubulysin m (POBZYODNVHQLFG-ZRBKHQLFSA-N); Tucatinib (SDEAXTCZPQIFQM-UHFFFAOYSA-N); Tucidinostat (SZMJVTADHFNAIS-BJMVGYQFSA-N); Tulobuterol hydrochloride (RSLNRVYIRDVHLY-UHFFFAOYSA-N); Turofexorate isopropyl (INASOKQDNHHMRE-UHFFFAOYSA-N); Tuxobertinib (HIBPKFXWOPYJPZ-UHFFFAOYSA-N); Tylocrebrine (YFEPHJVWLFGWKH-CQSZACIVSA-N); Tylophorine (SSEUDFYBEOIWGF-AWEZNQCLSA-N); Tyrphostin A23 (VTJXFTPMFYAJJU-UHFFFAOYSA-N); Tyrphostin B42 (TUCIOBMMDDOEMM-RIYZIHGNSA-N); U.S. Ser. No. 10/246,444, Example 92 (UVWRRZRGHPHHLL-QHCPKHFHSA-N); U.S. Ser. No. 10/266,549, Example 14 (VPALTONRQJGXKI-UHFFFAOYSA-N); U.S. Ser. No. 10/669,234, Example 122 (ITVHSCOFHWBGHL-KRWDZBQOSA-N); U.S. Pat. No. 9,107,954, maraviroc (GSNHKUDZZFZSJB-ILVMPNSOSA-N); Ubenimex (VGGGPCQERPFHOB-RDBSUJKOSA-N); Ucn-01 (PBCZSGKMGDDXIJ-KRUBCLEUSA-N); Ulixertinib (KSERXGMCDHOLSS-LJQANCHMSA-N); Unii-16720ver1H (JXDYOSVKVSQGJM-UHFFFAOYSA-N); Unii-2MT30ehi63 (LBEJYFVJIPQSNX-UHFFFAOYSA-N); Unii-4GF4A623RF (SCFMWQIQBVZOQR-UHFFFAOYSA-N); Unii-4T71DCOGOA (NNXDIGHYPZHXTR-ONEGZZNKSA-N); Unii-59784ZX4GD (QJTLLKKDFGPDPF-QGZVFWFLSA-N); Unii-6lgrOryy5Q (FSBMCTDYWXIBLM-UHFFFAOYSA-N); Unii-8KG95Q2dqh (JZARXLRJMQKPON-UHFFFAOYSA-N); Unii-9J13XU96Z1 (OXTSYWDBUVRXFF-GDLZYMKVSA-N); Unii-E3T5xxy9HX (KBQCEQAXHPIRTF-UHFFFAOYSA-N); Unii-E53VN70K2X (AKLBERUGKZNEJY-RTEPGWBGSA-N); Unii-IODX1FG55Y (ZGNKNLOBYFTGRG-GIWKVKTRSA-N); Unii-OW6LM47pnk (ITMGVSSHWMTJRR-UHFFFAOYSA-N); Unii-VI6E2D3cyp (VNLTWJIWEYPBIF-KMSLUKAPSA-N); Unii-czp9GB25HO (MWUFVYLAWAXDHQ-HMNLTAHHSA-N); Unii-mjw34hdb0D (JNEGMBHBUAJRSX-UHFFFAOYSA-N); Unk-4Abz-DL-Dab(Bz(2-CO2H))-OH·CH3CO2H (MMPPVXHIYQJYQT-UHFFFAOYSA-N); Unk-xiHyp-DL-N(Me)Leu-DL-xiThr(1)-Unk-Leu-Pro-N(Me)Tyr(Me)-(1) (RHKBSXYAXHKXEG-BBAJFZNZSA-N); Upjohn culture 135bt (WVIUOSJLUCTGFK-UHFFFAOYSA-N); Uprosertib (AXTAPYRUEKNRBA-JTQLQIEISA-N); Uracil mustard (IDPUKCWIGUEADI-UHFFFAOYSA-N); Urea, 1,4-cyclohexylenebis(3-(2-chloroethyl)-3-nitroso-, (Z)-(NLVXTJQYQLAKKJ-UHFFFAOYSA-N); Urethane (JOYRKODLDBILNP-UHFFFAOYSA-N); Ursodeoxycholic acid (RUDATBOHQWOJDD-UZVSRGJWSA-N); Ursolic acid (WCGUUGGRBIKTOS-GPOJBZKASA-N); Usnic acid (CUCUKLJLRRAKFN-UHFFFAOYSA-N); VER-49009 (HUNAOTXNHVALTN-UHFFFAOYSA-N); VLX-600 (UQOSBPRTQFFUOA-NHDPSOOVSA-N); VX-702 (FYSRKRZDBHOFAY-UHFFFAOYSA-N); Vactosertib (FJCDSQATIJKQKA-UHFFFAOYSA-N); Vadimezan (XGOYIMQSIKSOBS-UHFFFAOYSA-N); Valethamate bromide (CEJGGHKJHDHLAZ-UHFFFAOYSA-M); Valinomycin (FCFNRCROJUBPLU-UHFFFAOYSA-N); Valproate ion (NIJJYAXOARWZEE-UHFFFAOYSA-M) salts; Valproic acid (NIJJYAXOARWZEE-UHFFFAOYSA-N); Valrubicin (ZOCKGBMQLCSHFP-KQRAQHLDSA-N); Vandetanib (UHTHHESEBZOYNR-UHFFFAOYSA-N); Vanoxerine (NAUWTFJOPJWYOT-UHFFFAOYSA-N); Vanoxerine dihydrochloride (MIBSKSYCRFWIRU-UHFFFAOYSA-N); Vardenafil (SECKRCOLJRRGGV-UHFFFAOYSA-N); Varenicline (JQSHBVHOMNKWFT-UHFFFAOYSA-N); Varespladib (BHLXTPHDSZUFHR-UHFFFAOYSA-N); Varlitinib (UWXSAYUXVSFDBQ-CYBMUJFWSA-N); Vatalanib (YCOYDOIWSSHVCK-UHFFFAOYSA-N); Vatalanib dihydrochloride (AZUQEHCMDUSRLH-UHFFFAOYSA-N); Veham-Sandoz (NRUKOCRGYNPUPR-ANTWWVKXSA-N); Velban (KDQAABAKXDWYSZ-PNYVAJAMSA-N); Veliparib (JNAHVYVRKWKWKQ-CYBMUJFWSA-N); Vemurafenib (GPXBXXGIAQBQNI-UHFFFAOYSA-N); Venetoclax (LQBVNQSMGBZMKD-UHFFFAOYSA-N); Vengicide (XOKJUSAYZUAMGJ-UHFFFAOYSA-N); Venlafaxine (PNVNVHUZROJLTJ-UHFFFAOYSA-N); Vepesid j (VJJPUSNTGOMMGY-SEMSPOPGSA-N); Veralipride (RYJXBGGBZJGVQF-UHFFFAOYSA-N); Verapamil (SGTNSNPWRIOYBX-UHFFFAOYSA-N); Verapamil hydrochloride (DOQPXTMNIUCOSY-UHFFFAOYSA-N); Verdinexor (OPAKEJZFFCECPN-XQRVVYSFSA-N); Verrucarin A,10-epoxide (CLNDGDZYSDRNSO-JSGIOEDPSA-N); Verrucarin A,8-dihydroxy-7′-(1-hydroxyethyl)-(PYYBXMVTBWYBDY-KQQUZDAGSA-N); Verubulin (SNHCRNMVYDHVDT-UHFFFAOYSA-N); Vicriviroc (CNPVJJQCETWNEU-CYFREDJKSA-N); Victoria blue r (JEVGKYBUANQAKG-UHFFFAOYSA-N); Vidarabine (OIRDTQYFTABQOQ-UHTZMRCNSA-N); Vidofludimus (XPRDUGXOWVXZLL-UHFFFAOYSA-N); Vilazodone (SGEGOXDYSFKCPT-UHFFFAOYSA-N); Vinblastine (JXLYSJRDGCGARV-CFWMRBGOSA-N); Vinblastine (JXLYSJRDGCGARV-XQKSVPLYSA-N); Vinblastine sulfate (KDQAABAKXDWYSZ-FCIBFBFCSA-N); Vinblastine sulfate (KDQAABAKXDWYSZ-JKDPCDLQSA-N); Vincamine (RXPRRQLKFXBCSJ-GIVPXCGWSA-N); Vincristine (OGWKCGZFUXNPDA-XQKSVPLYSA-N); Vincristine sulfate (AQTQHPDCURKLKT-JKDPCDLQSA-N); Vindesine sulfate (COFJBSXICYYSKG-FJFFLIEUSA-N); Vindoline (CXBGOBGJHGGWIE-ACSXSLCXSA-N); Vinleurosine sulfate (OREMAKNOJRHDJT-SQUPYDECSA-N); Vinorelbine (GBABOYUKABKIAF-BXZSYHTRSA-N); Vinorelbine tartrate (CILBMBUYJCWATM-UDRBCWGGSA-N); Vinpocetine (DDNCQMVWWZOMLN-IRLDBZIGSA-N); Viridicatumtoxin (FNSQKFOXORBCCC-WBWZXODPSA-N); Vismodegib (BPQMGSKTAYIVFO-UHFFFAOYSA-N); Vistusertib (JUSFANSTBFGBAF-IRXDYDNUSA-N); Visudyne (YTZALCGQUPRCGW-ZSFNYQMMSA-N); Vitamin K1 (MBWXNTAXLNYFJB-NKFFZRIASA-N); Vitamin a palmitate (VYGQUTWHTHXGQB-FFHKNEKCSA-N); Vitamin e succinate (IELOKBJPULMYRW-NJQVLOCASA-N); Vitilevuamide (UFOVYHGILLJGLP-UHFFFAOYSA-N); Vlx1570 (SCKXBVLYWLLALY-CZCYGEDCSA-N); Volasertib (SXNJFOWDRLKDSF-XKHVUIRMSA-N); Voreloxin (XZAFZXJXZHRNAQ-STQMWFEESA-N); Vorinostat (WAEXFXRVDQXREF-UHFFFAOYSA-N); Vortioxetine (YQNWZWMKLDQSAC-UHFFFAOYSA-N); Voruciclib (MRPGRAKIAJJGMM-OCCSQVGLSA-N); Voxtalisib (RGHYDLZMTYDBDT-UHFFFAOYSA-N); Wander (UJQGBYRKCZQJJS-UHFFFAOYSA-N); Warfarin (PJVWKTKQMONHTI-UHFFFAOYSA-N); Wedelolactone (XQDCKJKKMFWXGB-UHFFFAOYSA-N); Withaferin a (DBRXOUCRJQVYJQ-CKNDUULBSA-N); Withanolide e (RUVPNJSJTWTANE-LFCBYZEKSA-N); Wln: L E6 C666 BV mvt&&&J DQ HQ H2 ivol OQ RQ FO-FT6otj B1 CQ DN1&1 (ZJBMQVPEJHVSQA-KNYUDZPRSA-N); XL-019 (ISOCDPQFIXDIMS-UHFFFAOYSA-N); XL019 (ISOCDPQFIXDIMS-QHCPKHFHSA-N); XL228 (ALKJNCZNEOTEMP-UHFFFAOYSA-N); XL388 (LNFBAYSBVQBKFR-UHFFFAOYSA-N); XL888 (LHGWWAFKVCIILM-LAQKFSSHSA-N); Xanthohumol (ORXQGKIUCDPEAJ-YRNVUSSQSA-N); Xevinapant (LSXUTRRVVSPWDZ-MKKUMYSQSA-N); Xipamide (MTZBBNMLMNBNJL-UHFFFAOYSA-N); Xylazine (BPICBUSOMSTKRF-UHFFFAOYSA-N); Xylometazoline (HUCJFAOMUPXHDK-UHFFFAOYSA-N); Y-27632 dihydrochloride (IYOZTVGMEWJPKR-VOMCLLRMSA-N); Yangonin (XLHIYUYCSMZCCC-VMPITWQZSA-N); Z-LLNle-CHO (RNPDUXVFGTULLP-VABKMULXSA-N); Z-Leu-leu-leu-al (TZYWCYJVHRLUCT-VABKMULXSA-N); ZINC20060174 ion (DCFHNESNKIDDCH-UHFFFAOYSA-N) salts; ZSTK474 (HGVNLRPZOWWDKD-UHFFFAOYSA-N); Zafirlukast (YEEZWCHGZNKEEK-UHFFFAOYSA-N); Zalcitabine (WREGKURFCTUGRC-POYBYMJQSA-N); Zaltoprofen (MUXFZBHBYYYLTH-UHFFFAOYSA-N); Zanubrutinib (RNOAOAWBMHREKO-QFIPXVFZSA-N); Zebularine (RPQZTTQVRYEKCR-WCTZXXKLSA-N); Zibotentan (FJHHZXWJVIEFGJ-UHFFFAOYSA-N); Zidovudine (HBOMLICNUCNMMY-XLPZGREQSA-N); Zileuton (MWLSOWXNZPKENC-UHFFFAOYSA-N); Zilpaterol (ZSTCZWJCLIRCOJ-DGCLKSJQSA-N); Zimeldine hydrochloride (WFUAVXYXJOQWAZ-GVKRCPFFSA-N); Zimelidine hydrochloride (GELMUARXROJGSO-LFMIJCLESA-N); Zinc pyrithione (OTPSWLRZXRHDNX-UHFFFAOYSA-L); Ziprasidone (MVWVFYHBGMAFLY-UHFFFAOYSA-N); Zoledronic acid (XRASPMIURGNCCH-UHFFFAOYSA-N); Zolmitriptan (ULSDMUVEXKOYBU-ZDUSSCGKSA-N); Zonisamide (UBQNRHZMVUUOMG-UHFFFAOYSA-N); Zorubicin hydrochloride (NKFHKYQGZDAKMX-CBGHEUOKSA-N); Zosuquidar (IHOVFYSQUDPMCN-XAKVHENESA-N); Zosuquidar trihydrochloride (ZPFVQKPWGDRLHL-ZLYBXYBFSA-N); Zotarolimus (CGTADGCBEXYWNE-JUKNQOCSSA-N); Zotatifin (QYCXWOACFWMQFO-WZWZCULESA-N); Zotepine (HDOZVRUNCMBHFH-UHFFFAOYSA-N); Zotiraciclib (VXBAJLGYBMTJCY-NSCUHMNNSA-N); Zygosporin a (SDZRWUKZFQQKKV-WISUYLHISA-N); [(2,3,4,5-Tetrachlorocyclopenta-2,4-dien-1-ylidene)methyl]benzene (UJVQTEILOLRAQA-UHFFFAOYSA-N); [(2S)-oxiran-2-yl]methyl 9-oxo-1OH-acridine-4-carboxylate (ZRIKPWLZQORDHP-JTQLQIEISA-N); [(Z)-(3-Aminopyridin-2-yl)methylideneamino]thiourea (XMYKNCNAZKMVQN-WCIBSUBMSA-N); [(Z)-[5-(trifluoromethyl)isoquinolin-1-yl]methylideneamino]thiourea (LVZDIKXEFVVOAF-FXBPXSCXSA-N); [1,2,5]Oxadiazolo[3,4-b]pyrazine-5,6-diamine, N,N′-bis-(4-fluorophenyl)-(YZIQCRJJYTYRFD-UHFFFAOYSA-N); [2-(1H-indol-4-yl)-1H-imidazol-4-yl](3,4,5-trimethoxyphenyl)methanone (WDMPGPZDSGODDF-UHFFFAOYSA-N); [2-(1H-indol-6-yl)-1H-imidazol-5-yl]-(3,4,5-trimethoxyphenyl)methanone (ZWYWKMGQZNGTAQ-UHFFFAOYSA-N); [4-(3,5-Dichlorophenyl)piperazin-1-yl]-(2-ethoxyphenyl)methanone (NSLOWXIBFUYJLC-UHFFFAOYSA-N); [4-(Nitrooxymethyl)phenyl]benzoate (TXDZRXFMCDVSAZ-UHFFFAOYSA-N); [5-(6-Aminopurin-9-yl)-3,4-dihydroxyoxolan-2-yl]methyl sulfamate (GNZLUJQJDPRUTD-UHFFFAOYSA-N); [5-Anilino-2-(4-methylphenyl)-1,3-oxazol-4-yl]-triphenylphosphanium ion (CIMCWWOXSPUYCD-UHFFFAOYSA-N) salts; [8-Chloro-2-(4-chlorophenyl)quinolin-4-yl](piperidin-2-yl)methanol (GOVNXGKKGVVELA-UHFFFAOYSA-N); alpha-Dihydroartemisinin (BJDCWCLMFKKGEE-KDTBHNEXSA-N); alpha-GalCer (VQFKFAKEUMHBLV-BYSUZVQFSA-N); alpha-Mangostin (GNRIZKKCNOBBMO-UHFFFAOYSA-N); alpha-Peltatin (JGGWNGRBXJWAOC-HKJPBSJPSA-N); alpha-Pinene (GRWFGVWFFZKLTI-UHFFFAOYSA-N); alpha-Thiodeoxyguanosine (SCVJRXQHFJXZFZ-UHFFFAOYSA-N); antineoplastic-643001 (NYUPFPFTYCWUEZ-YSMBQZINSA-N); aziridin-1-yl-N,N-bis(2-chloroethyl)phosphonamidic acid; cyclohexanamine (XXZPTFKAQUKVAZ-UHFFFAOYSA-N); bafilomycin A1 (XDHNQDDQEHDUTM-YJSQNSDISA-N); beta-Boswellic acid (NBGQZFQREPIKMG-PONOSELZSA-N); beta-Escin (AXNVHPCVMSNXNP-YSYFQUGBSA-N); beta-Guttiferin (GEZHEQNLKAOMCA-RRZNCOCZSA-N); beta-Lapachone (QZPQTZZNNJUOLS-UHFFFAOYSA-N); beta-Peltatin (HLBPOYVRLSXWJJ-PDSMFRHLSA-N); beta-Resorcylaldehyde,4-phthalazinediyldihydrazone (KHMFQNBWCPWXQN-ASIDMNOUSA-N); beta-Rhodomycin (KXFLRDGRXPJAFE-UHFFFAOYSA-N); beta-Sitosterol (KZJWDPNRJALLNS-VJSFXXLFSA-N); bmh-21 (BXYDVWIAGDJBEC-UHFFFAOYSA-N); c-Kit-IN-1 (WWOXKWLDMLMYQY-UHFFFAOYSA-N); carboxy-N,N,N-trimethylmethanaminium ion (KWIUHFFTVRNATP-UHFFFAOYSA-O) salts; cis-Dihydronarciclasine (SBTGHBALOCEVOR-UHFFFAOYSA-N); dimethyl-[2-phenyl-1-(3,4,5-trimethoxybenzoyl)oxybutan-2-yl]azanium(Z)-4-hydroxy-4-oxobut-2-enoate (FSRLGULMGJGKGI-BTJKTKAUSA-N); ethyl 3-(2-((4-(N-(hexyloxycarbonyl)carbamimidoyl)phenylamino)methyl)-1-methyl-N-(pyridin-2-yl)-1H-benzo[d]imidazole-5-carboxamido)propanoate (KSGXQBZTULBEEQ-UHFFFAOYSA-N); gamma-Oryzanol (FODTZLFLDFKIQH-FSVGXZBPSA-N); gamma-Tocopherol (QUEDXNHFTDJVIY-DQCZWYHMSA-N); ha14-1 (SXJDCULZDFWMJC-UHFFFAOYSA-N); hexadecylamine, n-diethyl-, hydrobromide (YYNIVGIURMZWQU-UHFFFAOYSA-N); m-DL-Sarcolysin (QDGAVODICPCDMU-UHFFFAOYSA-N); methanesulfonic acid; N-(3-morpholinopropyl)-1-nitro-acridin-9-amine (GUDAGJUXXXVEIS-UHFFFAOYSA-N); methyl N-[(E)-1-pyridin-2-ylethylideneamino]carbamodithioate (IFRMECQLFGLWCS-YRNVUSSQSA-N); methyl N-[6-(3-iodobenzoyl)-1H-benzimidazol-2-yl]carbamate (NQDGZMIFDRCSIT-UHFFFAOYSA-N); n-Hydroxy-7-pentofuranosyl-7 h-pyrrolo[2,3-d]pyrimidin-4-amine (AESDJLQYSFFKSC-UHFFFAOYSA-N); o6-Benzylguanine (KRWMERLEINMZFT-UHFFFAOYSA-N); p-Benzoyloxybenzylchloride (NFJDWPJJWRNWNF-UHFFFAOYSA-N); p-Phenylenediamine (CBCKQZAAMUWICA-UHFFFAOYSA-N); trans-Epoxysuccinyl-L-leucylamido(4-guanidino)butane (LTLYEAJONXGNFG-HBNTYKKESA-N); trans-Flupentixol (NJMYODHXAKYRHW-BLLMUTORSA-N), including synergy interactions between API and AAPI;

In another embodiment, provided are the method when the non-covalent complex or molecular/solid dispersion or conjugate is combined with at least one additional active pharmaceutical ingredient (AAPI) selected from:
A. inductor, inhibitor or modulator of P-glycoprotein/BCRP/MRP2/MCT1/SNAT2 PEPT1/ABC-transporters which are described in the scientific and patent literature, for example (non-limited examples):Curcumin, Demethoxycurcumin, Bisdemethoxycurcumin, and the like;
B. inductor, inhibitor or modulator of CYP1, CYP2, CYP3 enzymes (non limiting examples: CYP1A1, 1A2, 2A6, 2B6, 2C8, 2C9, 2C19, 2D6, 2E1 or 3A4 enzymes) which are described in the scientific and patent literature, for example (non-limited examples for CYP3A4): sulphaphenazole, quinidine, furafylline, troleandomycin (TAO) and diethyldithiocarbamate (DDC), and the like;
C. dipole modifiers that are able to incorporate into lipid membranes and modify their physicochemical properties, phase segregation character, dipole potential which are described in the scientific and patent literature, for example (non-limited examples): flavanoids, phloretin, floricin, biochanin A, myricetin, quercetin, taxifolin, genistin, genistein, daidzein, catechin, 2,4,6-trihydroxyacetophenone (THAP), floxin B and erythrosine, dihydroquercetin, and the like with or without vitamin C;
D. Hepatoprotective agent which are described in the scientific and patent literature, for example (non-limited examples): according to the different mechanism of action, it can be roughly divided into detoxification drugs (eg. N-acetylcysteine, S-adenosylmethionine and Glutathione), anti-inflammatory drugs (eg. Glycyrrhizic acid preparation, magnesium isoglycyrrhizinate), hepatocyte membrane protector (eg. Polyene phosphatidylcholine), antioxidant drugs (eg. Bicyclol, Silymarin), anticholestatic drug (Ursodeoxycholic acid, Cholestyramine), immunosuppressants (Glucocorticoids) and specific treatment agents (L-carnitine, Anticoagulants), and the like.

In another embodiment, provided are the method when the non-covalent complex or molecular/solid dispersion or conjugate is combined with at least one additional active pharmaceutical ingredient (AAPI) selected from:
A. autophagy inductor, inhibitor or modulator which are described in the scientific and patent literature, for example (non-limited examples): lithium; carbamazepine; Rapamyrin; Ternsirolimus; Everolimm; Oeforolimus; PP242; Torin 1; Metformin; 15132799; GDC-0980; 501-0941; Perifosine; Fluspirilene; CCE779; Pimozide; Trifluoperazine; Nicardipine; Lithium; L-690330; Carbamazepine; Xmtospongin B; Minoxidil; Clonidine; Verapamil; Loperamide; Amiodarone; Nimodipine; Nitrendipine; Rilmenidine; Bromperidol; Metergoline; Thioridazine; Chlorpromazine; Fludroconisone; Noscapine; Oemastine; Tat-beclin 1 peptide, and the like;
B. autoptosis inductor, inhibitor or modulator which are described in the scientific and patent literature.
C. ferroptosis inductor, inhibitor or modulator which are described in the scientific and patent literature;
D. pyroptosis inductor, inhibitor or modulator which are described in the scientific and patent literature;
E. cell reprogramming agent which are described in the scientific and patent literature;
F. SCOT inductor, inhibitor or modulator which are described in the scientific and patent literature;
G. senolitic agent which are described in the scientific and patent literature;
H. inductor, inhibitor or modulator of ABCA1; ABCB1; AHR; AIFM1; AKR1C₃; AKT1; ALOX12; ALOX5; ALPP; ANXA1; APAF1; AR; ATF2; ATF3; AURKB; BAD; BAK1; BAX; BBC3; BCL2; BCL2L1; BCL2L11; BCL2L2; BECN1; BID; BIRC2; BIRC3; birc5a; BNIP3; CALR; CASP12; CASP2; CASP3; CASP6; CASP7; CASP8; CASP9; CCNB1; CCND1; CCND2; CCND3; CCNE1; CD80; CDC25C; CDC42; CDH1; CDH2; CDK1; CDK2; CDK4; CDK6; CDK7; CDK9; CDKN1A; CDKN1B; CDKN1C; CDKN2B; CFLAR; CHEK-2; CHUK; COX-1; COX-2; CSN2; CTNNBIP1; CYC3B; CycA; CycB; CYCS; CYP19A1; CYP1A1; CYP1A2; CYP1B1; CYP2B6; CYP2C8; CYPIA1; DDIT3; DIABLO; Dox; DUSP10; E2F1; EBV-EA; EDN1; EGF; EGFR; EGR1; ENDOG; ERBB2; ERBB3; ERK1/2; ERP29; ESR1; FADD; Fas; FASLG; FGF2; FN1; FOS; FOXM1; GADD45A; GATA1; GATA2; GCLC; GGT1; GLI1; GPT; GPX1; GRB2; GSK3B; GSTM1; HBG1; HIF1A; HMBS; HNRNPA2B1; HSPA4; HSPA5; HSPB2; ICAM1; IFNG; IGF1; IGF1R; IGF2; IKBKB; IKBKE; IL1B; IL2; IL4; IL6; IL8; ITGA2; ITGAV; ITGB3; JAK1; JAK2; JUN; JUNB; KDR; KLK3; LDHB; LOX; LTA; LTA4H; MAP2; MAP2K1; MAP2K4; MAPK1; MAPK3; MAPK7; MAPK8; MCL1; MDM2; MMP1; MMP14; MMP15; MMP2; MMP3; MMP7; MMP9; MTOR; MYC; MYLK; NDUFS1; NFATC1; NFE2; NFKB1; NFKBIA; NOS2; NOX1; NQO1; NR1H3; ODC1; PARP1; PCNA; PDIA3; PHB; PIK3CA; PIM2; PLAU; PLCG1; PLK1; PMAIP1; POR; PPARG; PRKAB1; PRKACA; PRKCA; PRKCG; PROM1; Proteasome; PSMD9; PTCH1; PTGER3; PTK2; PTPN1; RAC1; RAD17; RAF1; RAP1A; RB1; RELA; RHOA; RHOB; ROCK1; ROS1; RPS6KA3; RPS6KA5; RPS6KB1; SLC2A1; SNAI1; SOD1; SOS1; SP1; SP3; SP4; SPDEF; SRC; STAT3; STAT5B; SULT1A3; Telomerase; TFDP1; TFF1; TGFBR1; TGFBR2; TGFBR3; TIMP1; TIMP2; TNF; TNFRSF10A; TNFRSF10B; TNFRSFlA; TNFRSF1B; TOP1MT; TOP2A; TOP2B; TP53; TP63; TRADD; TUBB3; TUBG1; TWIST1; UCP2; UGT1A; V-ATPases; VCAM1; VEGFA; WEE1; WSB1; XIAP; XIAP.

In another embodiment, provided are the method when the non-covalent complex or solid dispersion or conjugate is combined with at least one additional active natutral pharmaceutical ingredient (AANPI) active against disease and selected from: Terpenoids; Flavonoids; Alkaloids; Polyketides; Polycyclic aromatic natural products; Steroids; Saponin; Oxygen heterocycles; Benzopyranoids; Benzofuranoids; Aliphatic natural products; Aminoacids and peptides; Polypyrroles; Tannins;

and
(+)-(2R,3R)-Dihydrokaempferol; (+)-3′,4′,5,7-Tetrahydroxy-2,3-trans-flavan-3-ol; (+)-3-O-Acetyloleanolic acid; (+)-7-Oxo-13-epi-pimara-14,15-dien-18-oic acid; (+)-7-Oxo-13-epi-pimara-8,15-dien-18-oic acid; (+)-Acetyloleanolic acid; (+)-Altholactone; (+)-Aromadendrin; (+)-Berbamine; (+)-Byakangelicin; (+)-Caseamembrin A; (+)-Catechin; (+)-Chelidonine; (+)-Cularimine; (+)-Dihydrokaempferol; (+)-Funiferine; (+)-Gallocatechin; (+)-Hypopurin A; (+)-Ingenol 3,20-dibenzoate; (+)-Isocucurbitacin D; (+)-Isocupressic acid; (+)-Isopimaric acid; (+)-Isoplatydesmine; (+)-Isotetrandrinelsotetrandrine; (+)-Kahiricoside III; (+)-Kahiricoside IV; (+)-Kansenonol; (+)-Labda-7,12(E),14-triene-17-al; (+)-Labda-7,12(E),14-triene-17-ol; (+)-Longifolicin; (+)-Lucidenic acid N; (+)-Lucidenic acid P; (+)-Lupenol; (+)-Lushanrubescensin H; (+)-Luzonoid A; (+)-Luzonoid B; (+)-Luzonoid C; (+)-Luzonoid E; (+)-Marmesin; (+)-Matrine; (+)-Methyl lucidenate P; (+)-Methyl lucidenate Q; (+)-Methyl lucidenic acid F; (+)-Moroccolide A; (+)-Muricin A; (+)-Muricin B; (+)-Muricin D; (+)-Muricin F; (+)-Muricin H; (+)-Murrayacoumarin B; (+)-O-Methylthalicberine; (+)-Oleanolic acid acetate; (+)-Pancratistatin; (+)-Pinoresinol; (+)-S-Carvone; (+)-Salutaridine; (+)-Thalicarpine; (+)-Zerumin A; (+)-alpha-viniferin; (+)-beta,beta-Dimethylacrylshikonin; (+)-dihydroisorhamnetin; (+)-tylophorinidine; (+)Garcinialiptone A; (−)-10-Methoxycamptothecin; (−)-12-O-Methylvolkensin; (−)-Alkannin; (−)-Arctigenin; (−)-Burseran; (−)-Cephalotaxine; (−)-Deoxymannojirimycin; (−)-Deoxypodophyllotoxin; (−)-Deoxypodorhizone; (−)-Dicentrine; (−)-Epicatechin; (−)-Epigallocatechin; (−)-Falcarinol; (−)-Gossypol; (−)-Homopterocarpin; (−)-Interiorin B; (−)-Interiotherin D; (−)-Isoapetalic methyl ester; (−)-Isoquercetrin; (−)-Juncenolide A; (−)-Kansuiphorin A; (−)-Kaur-16-en-19-ol; (−)-Lariciresinol; (−)-Laxiflorin L; (−)-Lochnericine; (−)-Loliolide; (−)-Ludongnin J; (−)-Lungshengenin C; (−)-Lungshengenin G; (−)-Luzonoid D; (−)-Luzonoside A; (−)-Luzonoside B; (−)-Lycorine; (−)-Mangostenone C; (−)-Mangostenone D; (−)-Maoecrystal P; (−)-Melissoidesin G; (−)-Melissoidesin N; (−)-Melissoidesin 0; (−)-Melissoidesin S; (−)-Methylprotodioscin; (−)-N-Methyltaxol C; (−)-Naringenin; (−)-Nortrachelogenin; (−)-Orbiculatoside B; (−)-Paclitaxel; (−)-Perillylalcohol; (−)-Podophyllotoxin; (−)-Quercetin-3-O-beta-D-glucopyranoside; (−)-Secoisolariciresinol; (−)-Sonderianol; (−)-Swainsonine; (−)-Trachelogenin; (−)-Tubulosine; (−)-Tylocrebine; (−)-Wikstromol; (−)-Yatein; (−)-anonaine; (−)-ent-kaur-16-en-13,19-diol; (−)-eta-viniferin; (−)-kurarinone; (−)-usnic acid; (−)Cycloxanthochymol; (−)Garcinialiptone A; (1R,2S,4S,5R,7R,9S,1OR)-1-alpha-Benzoyloxy-2-alpha,15-diacetoxy-4-beta-hydroxy-9-beta-cinnamoyloxy-beta-dihydroagarofuran; (1S,2S,3R)-(+)-Isopicrodeoxypodophyllotoxin; (1S,5S,6R,7R,8R,9S,10S)-5-Angeloyloxy-8,9-epoxy-1-hydroxy-2-oxoxantha-3,11-dien-6,12-olide; (1S,5S,6R,7R,8S,10S)-5-Angeloyloxy-1,8-dihydroxy-2-oxoxantha-3,11-dien-6,12-olide; (1S,5S,6R,7S,10R)-1-Hydroxy-4-methoxy-5-methylbutanoyloxy-2,9-dioxoxanth-11-en-6,12-olide; (1S,5S,6R,7S,10S)-5-Angeloyloxy-1-hydroxy-2-oxoxantha-3,11-dien-6,12-olide; (1S,5S,6R,7S,9R,10S)-5-Methylbutanoyloxy-1,4,9-trihydroxy-2-oxoxanth-11-en-6,12-olide; (2,4-cis and trans)-gigantecinone; (2,4-cis and trans)-squamoxinone; (22R,23R,24R)-12-beta-acetyloxy-16-beta,23:22,25-diepoxy-23,24-dihydroxy-9,19-cyclolanostan-3-beta-yl alpha-L-arabinopyranoside; (23E)-coumaroylhederagenin; (25R)-5-alpha-spirostane-2-alpha,3-beta-diol 3-O—{O-beta-D-glucopyranosyl-(1->2)-O-[beta-D-xylopyranosyl-(1->3)]-O-beta-D-glucopyranosyl-(1->4)-beta-D-galactopyranoside}; (25R)-5-beta-spirostan-3-beta-ol 3-O-beta-D-glucopyranoside; (25S)-5-beta-spirostan-3-beta-ol 3-O-alpha-L-rhamnopyranosyl-(1->2)-[alpha-L-rhamnopyranosyl-(1->4)]-beta-D-glucopyranoside; (25S)-5-beta-spirostan-3-beta-ol 3-O-beta-D-glucopyranosyl-(1->2)-[beta-D-xylopyranosyl-(1->4)]-beta-D-glucopyranoside; (25S)-5-beta-spirostan-3-beta-ol 3-O-beta-D-glucopyranosyl-(1->2)-beta-D-6-O-acetylglucopyranoside; (25S)-5-beta-spirostan-3-beta-ol 3-O-beta-D-glucopyranosyl-(1->2)-beta-D-glucopyranoside; (25S)-neospirost-4-en-3-one; (2R)-7,4′-Dihydroxy-5-methoxy-8-methylflavan; (2R,3R)-2,3-Dihydro-3,5,7-trihydroxy-2-(4-hydroxyphenyl)-4H-1-benzopyran-4-one; (2R,3R)-3,5-dihydroxy-7-methoxy?avanone; (2R,3R)-Aromadendrin; (2R,3R)-Lespedezaflavanone C; (2S)-2′-methoxykurarinone; (2S)-5,7-Dimethoxy-8-(2S-hydroxy-3-methyl-3-butenyl)-flavanone; (2S)-5,7-Dimethoxy-8-formylflavanone; (2S)-5-hydroxy-7-methoxy-8-[(E)-3-oxo-1-butenyl]flavanone; (2S)-Abyssinone II; (3R,3′R,6′R)-beta,epsilon-Carotene-3,3′-diol; (3S)-3′,7-dihydroxy-2′,4′,5′,8-tetramethoxyisoflavan; (3S)-7-hydroxy-2′,3′,4′,5′,8-pentamethoxyisoflavan; (3S)-Methoxy-1,7-bis(4-hydroxyphenyl)-6E-hepten-5-one; (3S,5S)-3,5-dihydroxy-1,7-bis(4-hydroxyphenyl)heptane; (3S,5S)-3-Hydroxy-5-ethoxy-1-(4-hydroxyphenyl)-7-phenyl-6E-heptene; (3S,5S)-3-Hydroxy-5-methoxy-1-(4-hydroxyphenyl)-7-phenyl-6E-heptene; (4R,6R)-dihydroxy-4-[10(Z)-heptadecenyl]-2-cyclohexenone; (6R,7R,8R)-8-Methoxygoniodiol; (6S,7alphaR)-Loliolide; (7R,8S,7′R,8′R)-(+)-7′-acetylpicropodophyllin; (7S,8S,7'S,8'S)-3,3′,4′-trihydroxy-4-methoxy-7,7′-epoxylignan; (8′R)-neochrome; (8'S)-neochrome; (R)-(−)-Dicentrine; (R)-antofine; (R)-cryptopleurine; (R)-tylophorine; (S)-(+)-[6]Gingerol; (S)-5-hydroxy-7,4′-dimethoxyflavanone; (S)-6-Gingerol; (S)-isotylocrebrine; (S)-tyloindicine I; (S)-tylophorine; (all-E)-Lutein; 1′,2′,3′,4′-tetradehydrotubulosine; 1′-Acetoxychavicol acetate; 1′-Acetoxyeugenol acetate; 1,2,4-trihydroxyheptadec-16-ene; 1,2,4-trihydroxyheptadec-16-yne; 1,2,4-trihydroxynonadecane; 1,2-Di-O-(9Z,12Z,15Z-octadecatrienoyl)-3-O-(6-amine-6-deoxy-alpha-D-glucosyl)-glycerol; 1,2-Di-O-(9Z,12Z,15Z-octadecatrienoyl)-3-O-(6-p-hydroxy-phenyl-propionamido-6-deoxy-alpha-D-glucosyl)-glycerol; 1,2-Dihydro-1,2,3-trihydroxy-9-(4-methoxyphenyl)phenalene; 1,2-dihydrobis(de-O-methyl)-curcumin; 1,3,5-trihydroxy-4-methoxyxanthone; 1,3,6-Trihydroxy-2-methylanthraquinone; 1,3,7-Trihydroxyxanthone; 1,3-Dihydroxy-2-methoxymethylanthraquinone; 1,3-diacetylvilasinin; 1,4-Benzenediol; 1,5-Dihydroxy-3-methoxyxanthone; 1,5-Dihydroxyxanthone; 1,6-Dihydroxy-7-methoxy-8-(3-methylbut-2-enyl)6′,6′-dimethyl-pyrano(2′,3′:3,2)xanthone; 1,7-Bis(4-hydroxyphenyl)hepta-4E,6E-dien-3-one; 1-(7-hydroxy-2,6-dimethyl-1-naphthyl)-4-methyl-3-pentanone; 1-Deoxymannojirimycin; 1-O-Acetyl-9-O-demethylpluviine; 1-O-Acetylnorpluviine; 1-O-formyl-4′-demethoxy-3′,4′-methylenedioxy-methyl rocaglate; 1-Phenylhepta-1,3,5-triyne; 1-alpha,2-alpha,6-beta,15-tetraacetoxy-3-alpha-(alpha-methyl)-butanoyl-4-beta-hydroxy-9-beta-(beta-)furoyloxy-beta-dihydroagarofuran; 1-alpha,2-alpha,9-beta-Tri-(beta-)furoyloxy-4-beta-hydroxy-15-acetoxy-beta-dihydroagarofuran; 1-alpha,2-alpha-Di-(alpha-methyl)-butanoyl-4-beta-hydrooxy-9-beta-(beta-)furoyloxy-15-acetoxy-beta-dihydroagarofuran; 1-alpha-(alpha-Methyl0-butanoyl-2-alpha-(alpha-methyl)-propynoyloxy-4-beta-hydroxy-9-beta-(beta-)furoyloxy-15-acetoxy-beta-dihydroagarofuran; 1-beta,2-alpha-epoxytagitinin C; 1-beta,6-alpha-dihydroxy-4(15)-eudesmene; 1-cinnamoyl-3-acetyl-11-methoxymeliacarpinin; 1-cinnamoyl-3-hydroxy-11-methoxymeliacarpinin; 1-deoxy-3-methacrylyl-11-methoxymeliacarpinin; 1-hydroxy-2-hydroxymethyl-3-methoxyanthraquinone; 1-hydroxy-2-hydroxymethylanthraquinone; 1-hydroxy-3-methoxy-2-methylanthraquinone; 1-methoxyerythrabyssin II; 1-tigloyl-3,20-diacetyl-11-methoxymeliacarpinin; 10,13-dibenzoyltaxacustin; 10-Epi-olguine; 10-Gingerol; 10-Hydroxyasimicin; 10-Hydroxyglaucanetin; 10-Hydroxytrilobacin; 10-Methoxycamptothecin; 10-O-Methylmacluraxanthone; 11,15-Dioxo-3beta,7beta,12beta-trhydroxy-5alpha-lanost-8-en-24-oic acid; 11-Hydroxy-1-isomangostin; 11-Hydroxycanthin-6-one; 11-beta,13-dihydroparthenolide; 11-beta-hydroxycephalotaxine beta-N-oxide; 11-hydroxytephrosin; 12,15-cis-squamostatin-A; 12-Acetoxy-5-hydroxynerolidol; 12-Deoxyphorbol 13-(9,10-methylene)undecanoate; 12-Deoxyphorbol 20-acetate 13-angelate; 12-Deoxyphorbol 20-acetate 13-phenylacetate; 12-Hydroxychiloscyphone; 12-O-Methylcarnosicacid; 12-O-Methylvolkensin; 12-beta,21-dihydroxycimigenol 3-O-alpha-L-arabinopyranoside; 12-beta-hydroxycimigenol 3-O-alpha-L-arabinopyranoside; 12a-Hydroxypachyrrhizone; 13(R)-14(R)-hydroxyantofine-N-oxide; 13(R)-antofine-N-oxide; 13-Hydroxy-15-oxozoapatlin; 13-beta, 21-Dihydroxyeurycomanol; 14,15-beta-dihydroxyklaineanone; 14,15beta-Dihydroxyklaineanone; 14-Hydroxychaparrinone; 15,16-Dinor-8(17),11E-labdadien-13-one; 15-Hydroxy-8(17),13E-labdadien-15-oic acid; 15-alpha-Hydroxyholamine; 15-alpha-acetyl dehydrosulphurenic acid; 15-epi-4E-jatrogrossidentadion; 16-alpha-hydroxycleroda-3,13-dien-15,16-olide; 17-(methoxycarbonyl)-28-nor-isoiguesterin; 17-alpha-H-digitoxigenin; 17-alpha-H-periplogenin; 17-alpha-H-periplogenin-3-O-beta-d-cymaroside; 17-alpha-H-periplogenin-3-O-beta-d-digitoxoside; 17-alpha-H-periplogenin-beta-glucopyranosyl-(1->4)-2-O-acetyl-beta-digitalopyranoside; 17-beta-H-periplogenin-3-O-beta-d-digitoxoside; 17-epi-Holacurtine; 17-epi-N-Demethylholacurtine; 18-Hydroxywithanolide D; 18-beta-Glycyrrhetic acid; 18-beta-Glycyrrhetinic acid; 2′,3′,5,7-Tetrahydroxy flavone; 2′,4′-Dihydroxy-6′-methoxy-3′,5′-dimethylchalcone; 2′,4′-dihydroxychalcone; 2′,5,6′,7-Tetrahydroxy Flavanone; 2′,5,6′7-Tetrahydroxy flavone; 2′,5-Dihydroxy-6,7,8-trimethoxyflavone; 2′,6′-Dihydroxy-4′-hydroxyacetophenone; 2′,6′-dimethoxy-4,4′-dihydroxychalcone; 2′R,4′-hydroxyemoroidocarpan; 2(R)-hydroxybutanedioic acid; 2(R)-hydroxybutanedioic acid 1-methyl ester; 2,3-dihydro-3-beta-methoxyixocarpalactone A; 2,3-dihydro-3-beta-methoxyixocarpalactone B; 2,3-dihydroixocarpalactone B; 2,3-dihydrowithaferin A; 2,4′-dihydroxy-2′-methoxychalcone; 2,4′-dihydroxy-4-methoxydihydrochalcone; 2,4,4′-trihydroxydihydrochalcone; 2,4-Chebulyl-beta-D-glucopyranose; 2,6-Dimethoxy-p-benzoquinone; 2-(1-hydroxyethyl)naphtho[2,3-b]furan-4,9-quinone; 2-(2,4-Dihydroxyphenyl)-3,5,7-trihydroxy-4H-1-benzopyran-4-one; 2-(2,4-Dihydroxyphenyl)-5-hydroxy-8,8-dimethyl-3-(3-methyl-2-butenyl)-4H,8H-benzo[1,2-b:3,4-b′]dipyran-4-one; 2-(3,4-Dimethoxyphenyl)-3-hydroxy-5,6,7,8-tetramethoxy-4H-1-benzopyran-4-one; 2-(3,4-Dimethoxyphenyl)-5-hydroxy-6,7,8-trimethoxy-4H-1-benzopyran-4-one; 2-(3,4-Dimethoxyphenyl)-5-hydroxy-6,7-dimethoxy-4H-1-benzopyran-4-one; 2-(4-Hydroxyphenyl)naphthalic anhydride; 2-Hydroxyisojatrogrossidion; 2-Hydroxymethylanthraquinone; 2-Methyl-1,3,6-trihydroxy-9,10-anthraquinone; 2-Methyl-1,3,6-trihydroxyanthraquinone; 2-[10(Z)-heptadecenyl]-1,4-hydroquinone; 2-acetylfuro-1,4-naphthoquinone; 2-acetylnaphtho[2,3-b]furan-4,9-quinone; 2-alpha, 3-alpha, 19-beta, 23-beta-tetrahydroxyurs-12-en-28-oic acid; 2-alpha,3-beta,13-beta-trihydroxyurs-11-en-28-oic acid; 2-alpha,3-beta-Dihydroxy-D:A-friedoolean-28-oic acid; 2-alpha-acetoxy-2′beta-deacetylaustrospicatine; 2-alpha-hydroxy-3-beta-{[(2E)-3-phenyl-1-oxo-2-propenyl]oxy}-olean-12-en-28-oic acid; 2-alpha-hydroxy-3-beta-{[(2E)-3-phenyl-1-oxo-2-propenyl]oxy}urs-12-en-28-oic acid; 2-alpha-hydroxytirotundin; 2-alpha-hydroxyursolic acid; 2-deoxycucurbitacin D; 2-epi-hydroxy isojatrogrossidion; 2-methyl-1,3,6-trihydroxyanthraquinone; 2-methyl-3-hydroxy-4-pyrone; 20-epibryonolic acid; 20-hydroxy-12-deoxyphorbol angelate; 20-hydroxyresiniferol 9,13,-14-orthophenylacetate; 21-O-methyltoosendanpentol; 22-O-Methylprotodioscin; 22-epicalamistrin; 23,24-Dihydrocucurbitacin B; 23,24-Dihydrocucurbitacin E; 23,24-dihydro-25-acetylcucurbitacin F; 23,24-dihydrocucurbitacin F; 23-Hydroxy-3-beta-[(0-alpha-L-rhamnopyranosyl-(1->2)-[beta-D-glucopyranosyl-(1->4)]-alpha-L-arabinopyranosyl)oxy]lup-20(29)-en-28-oic acid; 23-Hydroxy-3-beta-[(O-alpha-L-rhamnopyranosyl-(1->2)-[beta-D-glucopyranosyl-(1->4)]-alpha-L-arabinopyranosyl)oxy]lup-20(29)-en-oic acid 28-O-alpha-L-rhamnopyranosyl-(1->4)-beta-D-glucopyranosyl-(1->6)-beta-D-glucopyranosyl ester; 23-Hydroxy-3-beta-[(O-alpha-L-rhamnopyranosyl-(1->2)-alpha-L-arabinopyranosyl)oxy]lup-20(29)-en-oic acid; 23-Hydroxy-3-beta-[(O-alpha-L-rhamnopyranosyl-(1->2)-alpha-L-arabinopyranosyl)oxy]lup-20(29)-en-oic acid 28-O-alpha-L-rhamnopyranosyl-(1->4)-O-beta-D-glucopyranosyl-(1->6)-beta-D-glucopyranosyl ester; 23-Hydroxy-3-beta-[(O-beta-D-glucopyranosyl-(1->3)-alpha-L-rhamnopyranosyl-(1->2)-alpha-L-arabinopyranosyl)oxy]lup-20(29)-en-28-oic acid; 23-Hydroxy-3-beta-[(O-beta-D-glucopyranosyl-(1->4)-alpha-L-arabinopyranosyl)oxy]lup-20(29)-en-28-oic acid; 24,25-dihydro-27-desoxywithaferin A; 24-Methylenecycloartan-3beta-ol; 24-Methylenecycloartanol; 24-Methylenecycloartanone; 24-Methylenecycloartenone; 24-epibrassinolide; 25-O-Methoxycimigenol 3-O-alpha-L-arabinopyranoside; 25-O-acetyl-12-beta-hydroxycimigenol 3-O-alpha-L-arabinopyranoside; 25-O-methoxycimigenol 3-O-alpha-L-arabinopyranoside; 25-acetylcucurbitacin F; 27-O-h-D-glucopyranosylviscosalactone B; 27-deoxyactein; 27-hydroxybullatacin; 28-deoxonimbolide; 28-homocastasterone; 28-hydroxyisoiguesterin; 28-oxoallobetulin; 2alpha-Hydroxytirotundin; 3″-hydroxy-4-epi-larreatricin; 3″-Methoxynyasol; 3′,4′,5,6,7,8-Hexamethoxyflavone; 3′-Hydroxy-5,6,7,8,4′-pentamethoxyflavone; 3′-Methoxypongapin; 3′-O-Methyl-6-(1,1-dimethylallyl)eriodictyol; 3′-O-methylviolanone; 3′-demethoxy-6-O-demethylisoguaicin; 3′-formyl-2′,4′,6′-trihydroxy-5′-methyldihydrochalcone; 3′-methoxypongapin; 3,22-dioxo-20-taraxastene; 3,3′,4′,5,6,7,8-heptamethoxyflavone; 3,3′-di-O-Methylquercetin; 3,4′-dihydroxy-5-methoxystilbene; 3,4-dehydrolarreatricin; 3,5′-dihydroxythalifaboramine; 3,5,7,2′,4′-Pentahydroxyflavonol; 3,5,7,3′,4′,5′-hexahydroxy flavanone-3-O-beta-D-glucopyranoside; 3,5,7,3′,4′-pentahydroxyflavone-3-O-beta-D-glucopyranoside; 3,5,7,3′,4′-pentahydroxyflavonol-3-O-beta-D-galactopyranoside; 3,5,7,3′,4′-pentahydroxyflavonol-3-O-beta-D-glucopyranoside; 3,5,7-Trihydroxy-2-[4-hydroxy-3-(3-methyl-2-butenyl)phenyl]-4H-1-benzopyran-4-one; 3,5,7-trihydroxyflavonol; 3,6,7,4′-Tetra-O-methyl-5,3′-dihydroxyflavone; 3,6-Epidioxy-1,10-bisaboladiene; 3,8-dihydroxy-9-methoxypterocarpan; 3,9-Dihydroxy-4-(3,3-dimethylallyl)[6aR,11aR]-pterocarpan; 3,9-Dihydroxycoumestan; 3,9-Dimethoxypterocarpan; 3-(4-Hydroxy-3-methoxyphenyl)-2-propenoic acid; 3-Beta-(E)-feruloyllupeol; 3-Hydroxy-3′,4′,5,6,7,8-hexamethoxyflavone; 3-Hydroxy-4-(3,3-dimethylallyl)-4″,5″-dehydropyrano(8,9:2″,3″][6aR,11aR]-pterocarpan; 3-O-Acetyloleanolic acid; 3-O-Acetylpadmatin; 3-O-Caffeoylquinic acid; 3-O-Methylquercetin; 3-O-[alpha-L-Arabinopyranosyl-(1->2)-alpha-L-arabinopyranosyl-(1->6)-2-acetamido-2-deoxy-beta-D-glucopyranosyl]oleanolic acid; 3-O-[alpha-L-rhamnopyranosyl(1->2)-beta-D-glucopyranosyl(1->2)-beta-D-glucuronopyranosyl]olean-12-en-22-oxo-3-beta,24-diol methyl ester; 3-O-[alpha-L-rhamnopyranosyl(1->2)-beta-D-glucopyranosyl(1->2)-beta-D-glucuronopyranosyl]olean-12-en-3-beta,22-beta,24-triol; 3-O-[alpha-L-rhamnopyranosyl(1->2)-beta-D-glucopyranosyl(1->2)-beta-D-glucuronopyranosyl]olean-12-en-3-beta,22-beta,24-triol methyl ester; 3-O-[beta-D-glucopyranosyl (1->3)-beta-D-galactopyranosyl(1->2)]-beta-D-glucuronopyranosyl-oleanolic acid-28-O-beta-D-glucopyranoside; 3-O-[beta-D-glucopyranosyl(1->2)-beta-D-galactopyranosyl(1->2)-beta-D-glucuronopyranosyl]olean-12-en-22-oxo-3-beta,24-diol methyl ester; 3-O-[beta-D-glucopyranosyl(1->2)-beta-D-galactopyranosyl(1->2)-beta-D-glucuronopyranosyl]olean-12-en-3-beta,22-beta,24-triol; 3-O-[beta-D-glucopyranosyl(1->2)-beta-D-galactopyranosyl(1->2)-beta-D-glucuronopyranosyl]olean-12-en-3-beta,22-beta,24-triol methyl ester; 3-O-[beta-D-glucopyranosyl(1->2)-beta-D-glucuronopyranosyl]olean-12-en-3-beta,22-beta,24-triol; 3-O-[beta-D-xylonopyranosyl-(1->2)-alpha-L-arabinopyranosyl-(1->6)-2-acetamido-2-deoxy-beta-D-glucopyranosyl]oleanolic acid; 3-O-[beta-D-xylopyranosyl-(1->2)-alpha-L-arabinopyranosyl-(1->6)-2-acetamido-2-deoxy-beta-D-glucopyranosyl]-21-O-trans-cinnamoylacacic acid; 3-O-alpha-L-Rhamnopyranosyl-(1->2)-[beta-D-glucopyranosyl-(1->4)]-alpha-L-arabinopyranosyl hederagenin 28-O-alpha-L-rhamnopyranosyl-(1->4)-beta-D-glucopyranosyl-(1->6)-beta-D-glucopyranosyl ester; 3-O-alpha-L-Rhamnopyranosyl-(1->2)-alpha-L-arabinopyranosyl hederagenin 28-O-alpha-L-rhamnopyranosyl-(1->4)-beta-D-glucopyranosyl-(1->6)-beta-D-glucopyranosyl ester; 3-O-beta-D-xylopyranosylstrophanthidin; 3-O-caffeoyl-quinic acid methyl ester; 3-Oxo-24-methylenecycloartane; 3-alpha-Amino-14-beta-hydroxypregnan-20-one; 3-alpha-acetoxydiversifolol; 3-beta,12-dihydroxy-13-methyl-6,8,11,13-podocarpatetraen; 3-beta,13-beta-dihydroxyurs-11-en-28-oic acid; 3-beta,15-alpha-dihydroxy lanosta-7,9(11),24-triene-21-oic acid; 3-beta,23,28-trihydroxy-12-oleanene 23-caffeate; 3-beta,23,28-trihydroxy-12-oleanene 3-beta-caffeate; 3-beta,23-epoxy-friedelan-28-oic acid; 3-beta-Hydroxy-27-p-(E)-coumaroyloxyurs-12-en-28-oic acid; 3-beta-Hydroxy-27-p-(Z)-coumaroyloxyolean-12-en-28-oic acid; 3-beta-Hydroxy-27-p-(Z)-coumaroyloxyurs-12-en-28-oic acid; 3-beta-O-(E)-coumaroylbetulin; 3-beta-O-(E)-feruloylbetulin; 3-beta-O-(alpha-L-rhamnopyranosyl-(1->2)-alpha-L-arabinopyranosyl)-hederagenin-28-O-(beta-D-glucopyranosyl-(1->4)-beta-D-glucopyranosyl)ester; 3-beta-O-(alpha-L-rhamnopyranosyl-(1->2)-alpha-L-arabinopyranosyl)-lup-20(29)-ene-28-O-beta-D-glucopyranosyl ester; 3-beta-O-(alpha-L-rhamnopyranosyl-(1->2)-alpha-L-arabinopyranosyl-olean-12-ene-28-O-(beta-D-glucopyranosyl-(1->4)-beta-D-glucopyranosyl)ester; 3-beta-O-(beta-D-glucopyranosyl-(1->3)-alpha-L-rhamnopyranosyl-(1->2)-alpha-L-arabinopyranosyl)-hederagenin-28-O-beta-D-glucopyranosyl ester; 3-beta-O-[alpha-L-rhamnopyranosyl-(1->2)-alpha-L-arabinopyranosyl]olean-12-ene-28-0-[alpha-L-rhamnopyranosyl-(1->4)-beta-D-glucopyranosyl-(1->6)-beta-D-glucopyranosyl]ester; 3-beta-O-[beta-D-glucopyranosyl-(1->3)-alpha-L-rhamnopyranosyl-(1->2)-alpha-L-ara-binopyranosyl]olean-12-ene-28-O-[alpha-L-rhamnopyranosyl(1->4)-beta-D-glucopyranosyl-(1->6)-beta-D-glucopyranosyl]ester; 3-beta-O-[beta-D-glucopyranosyl-(1->3)-alpha-L-rhamnopyranosyl-(1->2)-alpha-L-arabinopyranosyl]16R, 23dihydroxyolean-12-ene-28-O—{-alpha-L-rhamnopyranosyl (1->4)-[beta-D-quinovopyranosyl-(1->2)]-beta-D-glucopyranosyl-(1->6)-beta-D-glucopyranosyl]}ester; 3-beta-O-[beta-D-glucopyranosyl-(1->3)-alpha-L-rhamnopyranosyl-(1->2)-alpha-L-arabinopyranosyl]echinocystic acid28-O-[alpha-L-rhamnopyranosyl (1->4)-beta-D-glucopyranosyl-(1->6)-beta-D-glucopyanosyl]ester; 3-beta-O-[beta-D-glucopyranosyl-(1->3)-alpha-L-rhamnopyranosyl-(1->2)-alpha-L-arabinopyranosyl]olean-12-ene-28-O-[beta-D-glucopyranosyl-(1->3)-alpha-L-rhamnopyranosyl (1->4)-beta-D-glucopyranosyl-(1->6)-beta-D-glucopyranosyl]ester; 3-beta-O-[beta-D-quinovopyranosyl-(1->2)-alpha-L-arabinopyranosyl]hederagenin 28-O-[alpha-L-rhamnopyranosyl(1->4)-beta-D-glucopyranosyl-(1->6)-beta-D-glucopyranosyl]ester; 3-beta-O-[beta-D-quinovopyranosyl-(1->2)-alpha-L-arabinopyranosyl]olean-12-ene-28-O-[alpha-L-rhamnopyranosyl(1->4)-beta-D-glucopyranosyl-(1->6)-beta-D-glucopyranosyl]ester; 3-beta-O-alpha-L-arabinopyranosyl-lup-20(29)-ene-28-O-beta-D-glucopyranosyl ester; 3-beta-O-beta-D-xylopyranosyl-olean-12-ene-28-O-(alpha-L-rhamnopyranosyl-(1->4)-beta-D-glucopyranosyl-(1->4)-beta-D-glucopyranosyl)ester; 3-beta-[(0-alpha-L-Rhamnopyranosyl-(1->2)-[beta-D-glucopyranosyl(1->4)]-alpha-L-arabinopyranosyl)oxy]lup-20(29)-en-28-oic acid; 3-beta-[(O-beta-D-Glucopyranosyl-(1->3)-alpha-L-rhamnopyranosyl(1->2)-alpha-L-arabinopyranosyl)oxy]lup-20(29)-en-28-oic acid; 3-beta-acetoxy-12,19-dioxo-13(18)-oleanene; 3-beta-acetoxy-25-hydroxylanosta-8,23-diene; 3-beta-acetoxy-25-methoxylanosta-8,23-diene; 3-beta-acetoxy-8-beta-isobutyryloxyreynosin; 3-beta-o-(beta-D-glucopyranosyl-(1->3)-alpha-L-rhamnopyranosyl-(1->2)-alpha-L-arabinopyranosyl)-hederagrnin-28-O-(beta-D-glucopyranosyl-(1->4)-beta-D-glucopyranosyl)ester; 3-beta-trans-(3,4-dihydroxycinnamoyl-oxy)olean-12-en-28-oic acid; 3-beta-trans-feruloyloxy-16-beta-hydroxylup-20(29)-ene; 3-beta-trans-p-coumaroyloxy-2-alpha-hydroxyolean-12-en-28-oic acid; 3-beta-trans-p-coumaroyloxy-2-alpha-hydroxyurs-12-en-28-oic acid; 3-beta-trans-sinapoyloxylup-20(29)-en-28-ol; 3-epi-betulinic acid acetate; 3-hydroxy-2,4-dimethoxybenzaldehyde; 3-hydroxy-6′-desmethyl-9-O-methylthalifaboramine; 3-hydroxythalifaboramine; 3-isomangostin; 3-methoxy-4-hydroxybenzoic acid; 3-methylbut-2-enyl caffeate; 3-o-(cis-p-coumaroyl)-alphitolic acid; 3-o-(cis-p-coumaroyl)-maslinic acid; 3-o-(trans-p-coumaroyl)-alphitolic acid; 3-o-(trans-p-coumaroyl)-maslinic acid; 3-oxo-friedelan-28 oic acid; 3-tigloyl-1,20-diacetyl-11-methoxy-meliacarpinin; 3R)-6,4′-Dihydroxy-8-methoxyhomoisoflavan; 3R,8R-Dehydrofalcarindiol; 3beta,12-dihydroxy-13-methyl-5,8,11,13-podocarpatetraen-7-one; 3beta-Acetoxyolean-12-en-28-oic acid; 3beta-O-(2-Methylbutyryl)moroccolide A; 4′,5″-dihydroxy-6″-methoxy-4″,4″-dimethyl-4″,5″-dihydro-6″H-pyrano[2″,3″:7,8]-isoflavone; 4′,5′-Dihydro-11,5′-dihydroxy-4′-methoxytephrosin; 4′,5,6,7,8-Pentamethoxyflavone; 4′-Demethyldeoxypodophyllotoxin; 4′-Demethylpodophyllotoxin; 4′-Hydroxy wogonin; 4′-Hydroxy-3,5-dimethoxystilbene; 4′-O-methylellagic acid 3-(2″,3″-di-O-acetyl)-alpha-L-rhamnoside; 4′-bromoflavone; 4′-demethoxy-3′,4′-methylene-dioxy-methyl rocaglate; 4′-demethyldeoxypodophyllotoxin; 4′-demethylpodophyllotoxin; 4′-hydroxy-2,4-dimethoxydihydrochalcone; 4′-hydroxy-5,6-dehydrokawain; 4′-hydroxy-7-methoxyflavan; 4,4′-Dihydroxy-7,7′-dioxolign-8(8′)-ene; 4,4′-O-dimethylellagic acid 3-(2″,3″-di-O-acetyl)-alpha-L-rhamnoside; 4,4′-dihydroxy-2,6-dimethoxydihydrochalcone; 4-(1-hydroxy-2,2-dimethylcyclpropanone)-2,3-dihydrowithaferin A; 4-Allylveratrole; 4-Demethyl-podophyllotoxin; 4-Gingerol; 4-Hydroxycinnamic acid methyl ester; 4-Methoxydianthramide B; 4-Methylsulfinylbutyl isothiocyanate; 4-alpha,10-alpha-dihydroxy-3-oxo-8-beta-isobutyryloxyguaia-11(13)-en-12,6-alpha-olide; 4-alpha,6-alpha-dihydroxyeudesman-8-beta,12-olide; 4-beta,7-beta,20R-trihydroxy-1-oxowitha-2,5-dien-22,26-olide; 4-beta-D-(glucopyranosyloxy)-3-[3-methoxy-trans-isopenten-1-yl]acetophenone; 4-deoxyannoreticuin; 4-deoxyasimicin; 4-deoxygigantecin; 4-epi-Kaurenic acid; 4-epi-larreatricin; 4-hydroxy-2,6,4′-trimethoxydihydrochalcone; 4-hydroxy-3-methoxy-8,9-methylenedioxypterocarpan; 4-hydroxy-5-methoxycanthin-6-one; 4-hydroxyacetophenone 4-O-(6′-O-beta-D-apiofuranosyl)-beta-D-glucopyranoside; 4-methoxycarbonyl-5,10-benzogquinolinequinone; 4-nerolidylcatechol; 4E-jatrogrossidentadion; 4Z-jatrogrossidentadion; 5′-Demethoxydeoxypodophyllotoxin; 5′-hydroxythalifaboramine; 5,4′-Dihydroxy-4″,4″-dimethyl-5″-methyl-5″-H-dihydrofuruno[2″,3″:6,7]flavonone; 5,6,7-Trihydroxyflavone; 5,6-dehydrokawain; 5,6-dimethoxy-1-indanone; 5,7,2′,4′-Tetrahydroxy-8-prenylflavanone; 5,7,3′,4′-Tetrahydroxyisoflavone; 5,7,3′,4′-tetrahydroxyisoflavone; 5,7,3′-tri-O-methyl(−)-epicatechin; 5,7,4′-Trihydroxyisoflavone; 5,7,4′-trihydroxy-3,8,3′-trimethoxyflavone; 5,7,4′-trihydroxy-3,8-dimethoxyflavone; 5,7,4′-trihydroxy-6-methoxyhomoisoflavanone; 5,7,4′-trihydroxyhomoisoflavanone; 5,7-Dihydroxy-2-phenyl-4H-1-benzopyran-4-one; 5,7-Dihydroxy-6-(3-methylbut-2-enyl)-8-(2-methylbutyryl)-4-phenylcoumarin; 5,7-Dihydroxy-8-(2-hydroxy-3-methyl-3-butenyl)-4′-(1-(hydroxymethyl)pentacosyloxy)-6-prenylisoflavone; 5,7-Dihydroxy-8-(3-methyl-2-butenyl)-6-(3-methylbutyryl)-4-phenylcoumarin; 5,7-Dihydroxyflavone; 5,7-dihydroxyflavanone; 5,7-dimethoxy-3′,4′-methylenedioxyflavanone; 5,7-dimethyl-3′,4′-methylene(−)-epicatechin; 5-Hydroxy-1,4-naphthoquinone; 5-Hydroxy-2-(3-hydroxy-4-methoxyphenyl)-3,6,7-trimethoxy-4H-1-benzopyran-4-one; 5-Hydroxy-6,7,3′,4′-tetramethoxyflavone; 5-Hydroxy-6,7,8,3′,4′-pentamethoxyflavone; 5-Hydroxyseselin; 5-Methoxyseselin; 5-O-Methylembelin; 5-O-beta-D-galactopyranosyl-4-(4-hydroxyphenyl)-7-methoxy-2H-chromen-2-one; 5-O-beta-D-glucopyranosyl-4-(4-hydroxyphenyl)-7-methoxy-2H-chromen-2-one; 5-O-caffeoylshikimic acid; 5-desmethylnobiletin; 5-desmethylsinensetin; 5-epi-Vibsanin C; 5-epi-vibsanin H; 5-hydroxy-3-amino-2-aceto-1,4-naphthoquinone; 5-hydroxy-6-methoxyonychine; 5-hydroxybowdichione; 5-hydroxysophoranone; 5-methoxypsoralen; 5alpha-Hydroxytriptonide; 5alpha-O-(3′-dimethylamino-3′-phenylpropionyl) taxinine M; 6″-O-Xylosylglycitin; 6″-O-Xylosyltectoridin; 6″,6″-Dimethylpyrano[2″,3″:4′,3′]-2′,6′,beta-trimethoxychalcone; 6′-Hydroxyyatein; 6′-desmethylthalifaboramine; 6,3′,5′-Trihydroxy-2-phenylbenzofuran; 6,4′-dihydroxy-3′-methoxyaurone; 6,4′-dihydroxy-7-methoxyhomoisoflavan; 6,7-dimethoxycoumarin; 6,8-Diprenyleriodictyol; 6-(1,1-Dimethylallyl)eriodictyol; 6-(1,1-Dimethylallyl)naringenin; 6-(2-Hydroxy-3-methyl-3-butenyl)-8-prenyl-eriodictyol; 6-C-beta-D-Glucopyranosylapigenin; 6-Deoxyribaline; 6-Gingerol; 6-Hydroxyluteolin 6,7,3′,4′-tetramethyl ether; 6-Hydroxyrubiadin; 6-Methoxy-7-geranyloxycoumarin; 6-Methoxyaurapten; 6-Methyl-7-(3-oxobutyl)-bicyclo[4.1.0]heptan-3-one; 6-O-desmethylantofine; 6-O-desmethylsecoantofine; 6-Shogaol; 6-alpha-hydroxyneopulchellin; 6-beta-D-Glucopyranosyl-4′,5,7-trihydroxyflavone; 6-beta-D-Glucopyranosyl-5,7-dihydroxy-2-(4-hydroxyphenyl)-4H-1-benzopyran-4-one; 6-dehydroxylongilactone; 6-hydroxy-3-methoxytremetone; 6-hydroxycalyxin F; 6-methoxy-tremetone; 6-methyl-4-chromanone; 6-oxo-pristimerol; 6a,12a-dehydro-alpha toxicarol; 6beta-Cinnamoyl-7beta-hydroxyvouacapen-5alpha-ol; 7″-methylagathisflavone; 7,13-Abietadien-18-oic acid; 7,4′-Dihydroxyisoflavone; 7,4′-dihydroxy-8-methylflavan; 7,4′-dihydroxyflavan; 7,4′-dihydroxyhomoisoflavan; 7,4′-dihydroxyhomoisoflavanone; 7,7″-dimethyllanaraflavone; 7,9-dibenzoyltaxumairol P; 7-Hydroxy-5,6,8,4′-tetramethoxyflavone; 7-Hydroxy-8-methoxy-3-(4′-hydroxybenzyl)coumarin; 7-Isopteropodine; 7-Methoxypraecansone B; 7-O-Geranylscopoletin; 7-O-Methyleriodictyol; 7-O-methylrosmanol; 7-OH-flavanone; 7-[[2-(6-Deoxy-alpha-L-mannopyranosyl)-beta-D-glucopyranosyl]oxy]-5-hydroxy-2-(3-hydroxy-4-methoxyphenyl)-4H-1-benzopyran-4-one; 7-desmethyltylophorine; 7-epi-7-O-methylrosmanol; 7-hydroxy-3,4-dihydrocadalin; 7-hydroxycadallin; 7-methoxy-beta-carboline-1-propionic acid; 7-methoxypraecansone B; 8,14-epoxide; 8-(3,3-Dimethylallyl)spatheliachromene; 8-D-Glucosyl-4′,5,7-trihydroxyflavone; 8-Desoxygartanin; 8-Gingerol; 8-Hydroxyanthotoxol; 8-Prenyl erythrinin C; 8-beta-D-Glucopyranosyl-5,7-dihydroxy-2-(4-hydroxyphenyl)-4H-1-benzopyran-4-one; 8-dihydrotrichothecinol A; 8-epi-helenalin; 8-prenyldaidzaein; 9,13-diacetyltaxumairol W; 9-Aminocamptothecin; 9-Berberoline; 9-Methoxycamptothecin; 9-Methoxycanthin-6-one; 9-Methoxycanthin-6-one 3-N-oxide; 9-Oxo-seco-ratiferolide-5-alpha-O-(2-methylbutyrate); 9-alpha-Hydroxy-seco-ratiferolide-5-alpha-O-(2-methylbutyrate); 9-alpha-Hydroxy-seco-ratiferolide-5-alpha-O-angelate; 9-alpha-hydroxy-1-beta,10-alpha-epoxyparthenolide; 9-alpha-hydroxyparthenolide; 9-beta-hydroxy-1-beta,10-alpha-epoxyparthenolide; 9-beta-hydroxyparthenolide; 9-hydroxycanthin-6-one; 9-methoxycanthin-6-one; 9-methoxycanthin-6-one 3N-oxide; Abietic acid; Acetylcaranine; Acetyloleanolic acid; Acronycine; Acrovestone; Aculeatin A; Aculeatin B; Aculeatin E; Aculeatin F; Aculeatol E; Acumitin; Adenanthin C; Adenanthin F; Adenine; Aescin; Aesculin; Agastenol; Agastinol; Agathisflavone; Agehoustin A; Agehoustin B; Agladupol A; Aglafolin; Agrimoniin; Ailanquassin A; Ailanthone; Alizarin; Alizarin yellow; Alkannin; Alkannin beta,beta-dimethylacrylate; Alkylresorcinol A; Alkylresorcinol B; Alkylresorcinol C; Allamandin; Allanxanthone C; Allicin; Allobetulin; Alnusiin; Aloe emodin; Aloe resin B; Aloe-emodin; Aloesin; Alpha-Curcumene; Alpha-Humulene; Alpha-Ionone; Alpha-Mangostin; Alpha-Pinene; Alpha-Terpineol; Alpha-hederin; Alpha-tomatine; Alpinumisoflavone; Alstonine; Altaicalarins A; Altaicalarins B; Altaicalarins C; Altaicalarins D; Altholactone; Alvaradoins E; Alvaradoins F; Alvaradoins G; Alvaradoins H; Alvaradoins I; Alvaradoins J; Alvaradoins K; Alvaradoins L; Alvaradoins M; Alvaradoins N; Amaryllisine; Amentoflavone; Amomol A; Amomol B; Amooranin; Amygdalin; Anacardic acid; Ananixanthone; Andrographolide; Annoglaucin; Annoglaxin; Annomuricine E; Annonacin; Annonacin A; Annoreticuin-9-one; Anopterine; Anthocyanin 1; Anthocyanin 2; Anthricin; Antofine; Apetalic acid; Apigenin; Apigenin 8-C-glucoside; Arabelline; Araliasaponin IX; Arctigenin; Arctiin; Ardisiphenol A; Ardisiphenol B; Ardisiphenol C; Argentinic acid A methyl ester; Argentinic acid B methyl ester; Argentinic acid C methyl ester; Argentinic acid D methyl ester; Argentinic acid E methyl ester; Argentinic acid F methyl ester; Argentinic acid G methyl ester; Argentinic acid H methyl ester; Aristolochic acid A; Aristolochic acid I; Arnidiol; Artelasticin; Artemetin; Artobiloxanthone; Artoindonesianin B; Artoindonesianin P; Artoindonesianins A; Artoindonesianins G; Artoindonesianins H; Artoindonesianins I; Artoindonesianins U; Artoindonesianins V; Artonol B; Asarinin; Ascorbic acid; Asiatic acid; Asimicin; Asimin; Asiminacin; Asiminecin; Asitrilobins A; Asitrilobins B; Asparagoside A; Assamicin III; Astilbin; Aucubin; Aucuparin; Auraptene; Austrobailignan 1; Baicalein; Baicalein 7-glucuronide; Baicalin; Balsaminapentaol A; Balsaminol A; Balsaminol B; Balsaminol F; Balsaminols C; Balsaminols D; Balsaminols E; Balsaminoside A; Baphinitone; Barbaloin; Benzaldehyde; Benzoic acid; Berbamine; Berberine; Berberrubine; Bergenin; Beta-Caryophyllene; Beta-Farnesene; Beta-Ionone; Beta-Mangostin; Beta-d-glucopyranosyl-oleanolate; Beta-lapachone; Beta-peltatin; Beta-sitosterol; Beta-sitosterol-3-O-beta-D-glucoside; Beta-sitosterol-3-O-glucoside; Betanin; Betulin; Betulinic acid; Bigelovin; Bilobol; Biochanin A; Bisabolene; Bisabolol; Bisabolone; Blepharocalyxins A; Blepharocalyxins B; Blepharocalyxins C; Blepharocalyxins D; Blepharocalyxins E; Brasiliensic acid; Bruceantin; Bruceoside C; Brucine; Brucine N-oxide; Brunneogaleatoside; Bucidarasin A; Bucidarasin B; Bucidarasin C; Bucidarasin D; Buddledin A; Buddledin B; Buddledin C; Bullatacin; Bullatacinone; Bullatetrocin; Bursehernin; Burttinone; Butein; Butyl p-tolyl sulfide; Butylated hydroxyanisole; Butyrylmallotochromene; Byakangelicin; Cabraleadiol; Cabraleahydroxylactone; Cabralealactone; Caffeic acid; Caffeine; Cajanol; Calafatimine; Calamistrin A; Calamistrin B; Calamistrin C; Calamistrin D; Calamistrin E; Calamistrin F; Calamistrin G; Calaustralin; Calceolarioside A; Calceolarioside B; Calenduloside F6′-O-n butyl ester; Calenduloside G6′-O-methyl ester; Calophyllolide; Caloxanthone A; Calyxins A; Calyxins B; Calyxins C; Calyxins D; Calyxins E; Calyxins F; Calyxins G; Calyxins H; Calyxins I; Calyxins J; Calyxins K; Calyxins L; Campestrol; Camphene; Camptothecin; Candol B; Cantharidin; Canthin-6-one; Capillarisin; Capsaicin; Caracasine; Caracasine acid; Caribine; Carinatine; Carnosic acid; Carnosol; Carolin A; Carolin B; Carolin C; Carvacrol; Carvyl acetate; Caseamembrin A; Casearborin A; Casearborin B; Casearborin C; Casearborin D; Casearborin E; Casticin; Catechin; Caulophyllogenin; Cauloside D; Cedronolactones A; Cedronolactones B; Cedronolactones C; Cedronolactones D; Celastrol; Cephalomannine; Cephalotaxine; Cephalotaxine alpha-N-oxide; Cephalotaxine beta-N-oxide; Cepharanthine; Chamaejasmine; Chaparrin; Chaparrinone; Chartreusin; Chebulinic acid; Chelerythrine; Chelidonin; Chelidonine; Chlorogenic acid; Chromomycin; Chrysin; Chrysoeriol; Chrysophanol; Cicutoxin; Cimiracemoside B; Cineole; Cinnamaldehyde; Cinnamic acid; Cinnamyl alcohol; Cirsimaritin; Cis-4-deoxyannoreticuin; Cis-Zeatin; Citral; Citronellal; Citronellol; Citronellyl propionate; Cleomiscosin A; Clerodane diterpenoid; Cnicin; Congmunoside IX; Cordycepin; Coriadienin; Coriaheptocin A; Coriaheptocin B; Coriariin A; Corosolic acid; Corydaline; Costunolide; Coumarin; Coumestrol; Cresol; Crinamine; Crinasiadine; Crinasiatine; Crocetin; Crocin; Cryptolepine; Cucurbalsaminol A; Cucurbita-5,23(E)-diene-3-beta,7-beta,25-triol; Cucurbitacin A; Cucurbitacin B; Cucurbitacin D; Cucurbitacin E; Cucurbitacin F; Cucurbitacin I; Cucurbitacin J; Cucurbitacin Q; Cudraxanthone G; Cularimine; Cumestrol; Cumingianol A; Cumingianol B; Cumingianol D; Cumingianol F; Cumingianoside R; Curcumin; Curcusone A; Curcusone B; Curcusone C; Curcusone D; Curcusone E; Cussonoside B; Cyclamin; Cycleanine; Cycloartobiloxanthone; Cyclofoveoglin; Cycloxanthochymol; D-Catechin; D-Catechol; D-Limonene; D-alpha-tocotrienol; D-delta-tocotrienol; D-gamma-tocotrienol; Daidzein; Dalparvinene; Dalparvinene B; Dalparvone; Dalparvone B; Dalrubone; Daphneticin; Dasytrichone; Dawoensin A; Deguelin; Dehydrocostuslactone; Dehydrocycloxanthohumol; Dehydrodieugenol; Dehydroeburicoic acid; Dehydrofalcarindiol; Dehydrofalcarinol; Dehydrosulphurenic acid; Dehydrotomatine; Delphinidin; Demethylcalabaxanthone; Demethylencecalin; Demethylnobiletin; Dendroarboreols A; Dendroarboreols B; Deoxyandrographolide; Deoxycalyxin A; Deoxypodophyllotoxin; Desacetyluvaricin; Desmethylxanthohumol; Dexcyanidanol; Diacetylphiladelphicalactone C; Diacetylwithaferin A; Diallyl trisul?de; Diallyldisulfide; Diallylsulfide; Diarctigenin; Dielsiquinone; Differenol A; Digiferrugineol; Digiferruginol; Digitoxigenin; Digitoxigenin-beta-D-glycosyl-beta-D-digitaloside-beta-D-cymaropyranoside; Digitoxin; Digoxigenin; Digoxin; Dihydro costunolide; Dihydroanhydropodorhizol; Dihydrokawain; Dinoxin B; Diosgenin; Diosmetin 7-neohesperidoside; Diosmin; Diospyrin; Diphyllin; Dipropyl disulfide; Diuvaretin; Diynene; Doxorubicin; Drummondin A; Duartin; Dysosmarol; Eichlerialactone; Elagostasine; Elenoside; Ellagic acid; Ellipticine; Emarginatine F; Emarginatine G; Emetine; Emodin; Emoroidocarpan; Emorydone; Encecalin; Englerin A; Ent-14-beta-Hydroxykaur-16-en-15-one; Ent-18-Hydroxykaur-16-ene; Ent-7-alpha,14-beta-dihydroxykaur-16-en-15-one; Ent-7-alpha-Acetoxy-14-beta-hydroxykaur-16-en-15-one; Ent-7-alpha-Hydroxykaur-16-en-15-one; Ent-8,9-Seco-7-alpha,11-beta-dihydroxykaura-8(14),16-dien-9,15-dione; Ent-8,9-Seco-7-alpha-acetoxy-11-beta-hydroxykaura-8(14),16-dien-9,15-dione; Ent-8,9-Seco-7-alpha-acetoxykaura-8(14),16-dien-9,15-dione; Ent-8,9-Seco-7-alpha-hydroxy-11-acetoxykaura-8(14),16-dien-9,15-dione; Ent-8,9-Seco-7-alpha-hydroxy-17-thiophenylkaur-8(14)-en-9,15-dione; Ent-Kaur-16-en-15-one; Epi-betulinic acid; Epiashantin; Epicalyxins B; Epicalyxins C; Epicalyxins D; Epicalyxins F; Epicalyxins H; Epicalyxins I; Epicalyxins K; Epigallocatechin; Epigallocatechin gallate; Epipervilline; Epiyangambin; Epoxymollugin; Epoxymurin-A; Ergolide; Eriocitrin; Eriodictyol; Erlangerins A; Erlangerins B; Erybraedin A; Erybreadin B; Erybreadin C; Erycristagallin; Erysenegalensein E; Erysoline; Erystagallin A; Erysubin F; Erythrabissin-1; Erythrabyssin II; Erythribyssins A; Erythro-austrobailignan-6; Erythrodiol; Escobarine A; Escobarine B; Esculin; Ethoxychelerythrine; Ethyl gallate; Euclein; Eugenol; Eugenol methyl ether; Euparin; Eupatorin; Euxanthone; Evodiamine; F-Gitonin; Fagaronine; Falcarindiol; Falcarinol; Fangchinoline; Farnesiferol C; Farnesol; Fenchol; Fenchone; Ferruginin A; Ferruginin B; Ferruginin C; Ferruginol; Ferulic acid; Fisetin; Flavone; Flossonol; Folitenol; Foveoglin A; Foveoglin B; Friedelan-1,3-dione; Friedelin; Fumaric acid; Funiferine; Furomollugin; G-Mangostin; Gabunamine; Gabunine; Galanal B; Galangin; Galbacin; Galbelgin; Gallic acid; Gallogen; Gambogic acid; Gambogoic acid; Gamma-Tocopherol; Garcidepsidone A; Garcidepsidone B; Garcidepsidone C; Garcimangosxanthone A; Garcimangosxanthone B; Garcimangosxanthone C; Garcinialiptone B; Garcinialiptone C; Garcinialiptone D; Garcinone C; Garcinone D; Garcinone E; Gartanin; Gemin A; Genipin; Geniposide; Genistein; Genisteol; Genistin; Geranic acid; Geraniol; Geranyl acetate; Geranyl acetone; Geranyl butyrate; Geranylhydroquinone; Gericudranin A; Gericudranin B; Gericudranin C; Giganin; Gigantecin; Giganteosides D; Giganteosides E; Ginkgetin; Ginkgoic acid; Ginkgol; Ginsenoside Rb1; Ginsenoside Rb2; Ginsenoside Re; Ginsenoside Rg3; Ginsenoside Rhl; Gitoxigenin; Gitoxin; Glabridin; Glacins A; Glacins B; Glaucarubinone; Glaucarubol; Glaucarubolone; Glaucocalyxin A; Glaziovine; Globulixanthone D; Glossogin; Glycitein; Glycitin; Glycyrrhetinic acid; Glycyrrhitinic acid; Glycyrrhizic acid; Glycyrrhizic acid ammonium salt; Glycyrrhizin; Gnaphaliin; Goniotetracin; Goniothalamicin; Goniothalenol; Gossypol; Gracillin; Guaiacylglycerol; Guanepolide; Guggulsterone; Guttiferone A; Guttiferone I; Haemaocorin; Harmaline; Harmalol; Harmane; Harmine; Harmol; Harringtonine; Hederacolchisides A; Hederacolchisides A1; Hederagenin; Hederagenin 28-O-beta-D-glucopyranosyl ester; Hederagenin 3-O-alpha-L-arabinopyranoside; Hederagenin 3-O-alpha-L-rhamnopyranosyl-(1->2)-[beta-D-glucopyranosyl-(1->4)]-alpha-L-arabinopyranoside; Hederagenin 3-O-alpha-L-rhamnopyranosyl-(1->2)-alpha-L-arabinopyranoside; Hederagenin 3-O-beta-D-glucopyranosyl-(1->3)-alpha-L-rhamnopyranosyl-(1->2)-alpha-L-arabinopyranoside; Hederagenin 3-O-beta-D-glucopyranosyl-(1->4)-alpha-L-arabinopyranoside; Hederagenin 3-O-beta-D-glucopyranosyl-(1->4)-beta-D-glucopyranosyl-(1->3)-alpha-L-rhamnopyranosyl-(1->2)-alpha-L-arabinopyranoside; Hederagenin 3-O—{O-alpha-L-rhamnopyranosyl-(1->2)-alpha-L-arabinopyranoside}; Hederagenin 3-O—{O-beta-D-glucopyranosyl-(1->3)-O-alpha-L-rhamnopyranosyl-(1->2)-alpha-L-arabinopyranoside}; Hederagenin 3-O—{O-beta-D-glucopyranosyl-(1->4)-alpha-L-arabinopyranoside}; Heliamine; Helichrysetin; Helicquinone; Heliettin; Heriguard; Hernone; Hesperetin; Hesperidin; Hexanorcucurbitacin D; Hiravanone; Hirsutrin; Hispidol B; Hispidulin; Holacurtine; Holacurtinol; Holamine; Homoharringtonine; Homovitexin; Honokiol; Hydrastine; Hydroquinone; Hydroxyanigorufone; Hypercalin B; Hyperforin; Hyperin; Hyperoside; Hypoepistephanine; Hypopurin A; Icogenin; Ilicic acid; Illudin S; Imperatorin; Indicanin D; Indicanine D; Indicine; Indicine N-oxide; Indigo red; Indigopurpurin; IndirubinCouroupitine B; Indole-3-carbinol; Indole-3-carboxylic acid; Inflexusin B; Ingenol 3,20-dibenzoate; Ingenol-3,20-dibenzoate; Inophylloidic acid; Inophyllum E; Inoscavin A; Integerrimine; Interiorin B; Interiotherin A; Interiotherin B; Interiotherin C; Interiotherin D; Inuviscolide; Irisquinone A; Isoacteoside; Isoalantolactone; Isoallantolactone; Isoalvaxanthone; Isoapetalic acid; Isoapetalic acid 5-O-acetate; Isoapetalic methyl ester; Isobavachin; Isobonducellin; Isobrucine N-oxide; Isobutyrylmallotochromene; Isocarthamidin; Isocephalotaxine; Isochamaejasmin; Isochamuvaretin; Isochamuvaritin; Isocryptomerin; Isocucurbitacin D; Isocupressic acid; Isodesacetyluvaricin; Isodihydrocostunolide; Isodispar B; Isodonal; Isodonoiol; Isofoveoglin; Isofraxidin; Isogambogenic acid; Isoguaiacin; Isoharringtonine; Isohelenin; Isohelenol; Isohelianol; Isoiguesterol; Isolicoflavonol; Isolinderalactone; Isoliquiritigenin; Isomangiferolic acid; Isomorellin; Isoobtusilactone; Isopimpinellin; Isoplatydesmine; Isopsoralen; Isopteropodine; Isoquercetin; Isoquercetrin; Isoquercitrin; Isorecedensolide; Isorhamnetin 3-O-beta-D-glucopyranoside; Isorhamnetin 3-O-beta-D-glucoside; Isorhamnetin 3-O-glucoside; Isorhamnetin 3-glucoside; Isorhoifolin; Isorosmanol; Isoscutellarein; Isosinomenin A; Isosinomenine A; Isostrychnine; Isostrychnine N-oxide; Isostrychnopentamine; Isotaxiresinol; Isotetrandrine; Isotrilobine; Isotylocrebrine; Isouvaretin; Isovitexin; Isoxanthochymol; Isoxanthohumol; Isoxanthohumol(Sophora); Ixocarpalactone A; Jacaranone; Jatrophatrione; Jatropholone; Jatrophone; Javanicolides C; Javanicolides D; Javanicoside B; Javanicoside C; Javanicoside D; Javanicoside E; Javanicoside F; Javoricin; Juglone; Juncenolide A; JuncenolideC; Jungermannenone A; Junosmarin; Justicidin A; Justicidin B; Justicidin D; Justiflorinol; Kadsulactone; Kadsulactone A; Kadsuric acid; Kadsurin; Kaempferidinidin; Kaempferol; Kaempferol 3-O-beta-D-glucopyranoside; Kaempferol 5-O-beta-D-glucopyranoside; Kaempferol-7-O-beta-d-glucoside; Kahiricoside III; Kahiricoside IV; Kahiricoside V; Kansenone; Kansenonol; Kansuinine B; Kansuiphorin A; Kansuiphorin B; Karavilagenin E; Katuranin; Kawain; Kazinol F; Kazinol J; Khellactone; Kokusaginine; L-(−)-N-Methylcrotsparine; L-Dicentrine; L-Epigallocatechin; L-Glaziovine; L-Mimosine; L-epicatechin; Labda-7,12(E),14-triene-17-al; Labda-7,12(E),14-triene-17-ol; Labiatenic acid; Lactucin; Lakoochin A; Lakoochin B; Lampterol; Lanosta-7,9(11),24-triene-3alpha,23-diol; Lantadene A; Lapachol; Lappadilactone; Lariciresinol; Lasiocarpine; Lasiodonin; Lasiokaurin; Lasiopulide; Laxiflorin J; Laxiflorin K; Laxiflorin L; Laxiflorin M; Leachianone A; Leachianone G; Lemmaphylla-7,21-dien-3beta-ol; Lemobiline; Lespedezaflavanone C; Leurosidine; Leurosine; Liatrin; Licarin A; Licochalcone A; Licochalcone C; Licochalcone E; Ligstroside; Ligustenin A; Limocitrin 3,5,7,4′-tetramethyl ether; Limonene; Limonin; Linalool; Linaloyl acetate; Linalyl acetate; Linamarin; Linixanthone A; Linixanthone B; Linixanthone C; Lipiferolide; Liquiritigenin; Liriodenine; Lobaric acid; Lobatosides B; Lobatosides C; Lobatosides D; Lobatosides E; Lochnericine; Loliolide; Longicalycinin A; Longifolicin; Longikaurin B; Longilactone; Longimicins A; Longimicins B; Longimicins C; Longimicins D; Lophopterol; Lucidenic acid A; Lucidenic acid C; Lucidenic acid N; Lucidenic acid P; Lucidin dimethylether; Lucidin omega-methyl ether; Ludongnin J; Ludovicin A; Ludovicin B; Ludovicin C; Lungshengenin A; Lungshengenin C; Lungshengenin G; Lup-20(29)-ene-lbeta,3beta-diol; Lup-20(29)-ene-3alpha,23-diol; Lup-20(29)-ene-3beta,24-diol; Lupenol; Lupeol; Lupeol acetate; Lupinifolin; Lushanrubescensin H; Lutein; Luteolin; Luxenchalcone; Luzonial A; Luzonial B; Luzonidial A; Luzonoid A; Luzonoid B; Luzonoid C; Luzonoid D; Luzonoid E; Luzonoside A; Luzonoside B; Lycopene; Lycoricidine; Lycoricidinol; Lycorine; Machilin A; Machilin F; Machilin G; Machillene; Macranthoside B; Macrocalin B; Magnolol; Mahanimbicine; Mahanimbine; Mahanine; Majarine; Majidine; Malacitanolide; Maleic acid monoethyl ester; Mallotochromene; Mallotophenone; Malvidin; Malvidin-3-glucoside; Mammea A/AA; Mammea A/AB cyclo D; Mammea A/BA; Mammea A/BB; Mammea A/BD; Mammearin A; Mammeisin; Mangostanin; Mangostanol; Mangostenone C; Mangostenone D; Mangostenone E; Mangostin; Mangostinone; Mansonon D; Mansonone D; Mansonone H; Maoecrystal P; Maoyecrystal I; Marcanines A; Marcanines B; Marcanines C; Marcanines D; Marmesin; Martynoside; Maslinic acid; Matairesinol; Matrine; Maytanbutacine; Maytanbutine; Maytanprine; Maytansine; Maytanvaline; Maytenfolone-A; Mefiavolkenin; Meliacinanhydride; Melianin B; Melianin C; Meliatoxin B1; Meliavolkinin; Melicarpine; Melisemine; Melissoidesin G; Melissoidesin N; Melissoidesin O; Melissoidesin S; Melissoidesins N; Melissoidesins O; Melissoidesins S; Membranacin; Menthol; Menthone; Mesaconitin; Meso-dihydroguaiaretic acid; Meso-monomethyl dihydroguaiaretic acid; Mesuaxanthone A; Methyl 12-oxo-8alpha,15-dihydroxyabiet-13-en-19-oate; Methyl 15-hydroxy-8alpha,12alpha-epidioxiabiet-13-en-19-oate; Methyl 4-hydroxy cinnamate; Methyl allyl disulfide; Methyl caffeate; Methyl gallate; Methyl ganoderate F; Methyl linoleate; Methyl lucidenate A; Methyl lucidenate E2; Methyl lucidenate L; Methyl lucidenate P; Methyl lucidenate Q; Methyl p-coumarate; Methyl p-hydroxycinnamate; Methyl protodioscin; Methyl zhankuic acid A; Methyl-4-O-methylgallate; Methyleugenol; Methylmitrekaurenate; Methylprotodioscin; Mexicanin E; Mexicanin I; Mezerein; Michelenolide; Micheliolide; Microhelenin A; Microhelenin C; Microlenin; Micromelin; Microstegiol; Mitrekaurenone; Molephantin; Molephantinin; Mollugin; Molvizarin; Momorcharaside A; Monocrotaline; Montacin; Montalicin A; Montalicin B; Montalicin C; Montalicin D; Montalicin E; Montalicin F; Montalicin I; Montalicin J; Moracin D; Moracin I; Moracin M; Moracin N; Morelensin; Morellic acid; Morin; Morindaparvin A; Moroccolide A; Morusin; Mosin B; Mosin C; Mosinone A; Mucronulatol; Mulberrochromene; Multidione; Multigilin; Multiradiatin; Muricapentocin; Muricatacin; Muricatetrocin A; Muricatetrocin B; Muricatetrocin C; Muricatetrocins A; Muricatetrocins B; Muricatocin A; Muricatocin B; Muricatocin C; Muricin A; Muricin B; Muricin C; Muricin D; Muricin F; Muricin H; Muricin I; Muricoreacin; Murihexocin C; Murrayacoumarin A; Murrayacoumarin B; Murrayacoumarin C; Myrcene; Myriceric acid C; Myricetin; Myricetin 3-O-alpha-rhamnoside; Myricetin 3-O-beta-glucuronide; Myristicin; Myrtenol; N-(4-hydroxyundecanoyl)anabasine; N-Demethylholacurtine; N-Isobutyldeca-trans-2-trans-4-dienamide; N-Methylcrotsparine; N-Methyltaxol C; N-n-octanoylnornicotine; NSC 189487; NSC 251699; Nagilactone B; Nagilactone C; Nagilactone D; Nagilactone E; Naphthoflavon; Narciclasine; Naringenin; Naringin; Narirutin; Natsudaidain; Nectandrin A; Nectandrin B; Neo-tanshinlactone; Neocalyxins A; Neocalyxins B; Neocryptolepine; Neodiosmin; Neoeriocitrin; Neohesperidin; Neoisostegane; Neojusticin A; Neoponcirin; Neoxanthin; Neritaloside; Nerol; Nerolidol; Neryl acetate; Nimbolide; Nitidine; Nobiletin; Nordihydroguaiaretic acid; Norerythrostachaldine; Normangostin; Norobtusifolin; Noroxylin; Norsantal; Noscapine; Nymphone; O,O′-Dimethylobamegine; O,O′-Dimethylstepholine; O,O-Dimethylobamegine; O,O-Dimethylstepholine; O-Methylberbamine; O-Methylptelofolonium; O-Methylthalicberine; Obovaten; Obovatifol; Ocimene; Octadeca-9,11,13-triynoic acid; Odoroside A; Odoroside H; Ohioensin A; Olean-12-en-2-alpha,3-beta-diol; Olean-12-en-3-alpha,24-diol; Olean-12-en-3-beta-ol; Oleandrigeninsarmentoside; Oleandrin; Oleanolic acid; Oleanolic acid 3-O-alpha-L-rhamnopyranosyl-(1->2)-[beta-D-glucopyranosyl-(1->4)]-alpha-L-arabinopyranoside; Oleanolic acid 3-O-beta-D-glucopyranosyl-(1->3)-alpha-L-rhamnopyranosyl-(1->2)-alpha-L-arabinopyranoside; Oleanolic acid 3-O—{O-alpha-L-rhamnopyranosyl-(1->2)-O-[beta-D-glucopyranosyl-(1->4)]-alpha-L-arabinopyranoside}; Oleanolic acid 3-O—{O-alpha-L-rhamnopyranosyl-(1->2)-alpha-L-arabinopyranoside}; Oleanolic acid 3-O—{O-beta-D-glucopyranosyl-(1->3)-O-alpha-L-rhamnopyranosyl-(1->2)-alpha-L-arabinopyranoside}; Oleanolic acid acetate; Olivacine; Orbiculatoside B; Orientanol c; Orobol; Oropheolide; Osthole; Ovatine; Oxoushinsunine; P-Cymene; P-Hydroxybenzoic acid; P-Mentha-2,8-dien-1-ol; P-hydroxymethoxybenzobijuglone; Paclitaxel; Palmitic acid; Panaxynol; Pancratistatin; Panduratin A; Papaverine; Parthenolid-9-one; Parthenolide; Patavine; Paucin; Pelargonidin; Pelargonidin-3,5-diglucoside; Pellitorine; Peonidin; Perillaldehyde; Perillyl alcohol; Periplocymarin; Periplogenin-3-O-[beta-D-glucopyranosyl-(1->4)-beta-D-cymaropyranoside]; Perseal C; Perseal D; Petunidin; Peucedanin; Phaeantharine; Phenoxodiol; Pheophorbide a; Philadelphicalactone A; Philadelphicalactone C; Philadelphicalactone D; Phloroglucinol; Phyllamyricin C; Phyllanthostatin A; Physagulin D; Physagulin D(1->6)-beta-D-glucopyranosyl-(1->4)-beta-D-glucopyranoside; Physostigmine; Phytol; Piceatannol; Pinocembrin; Pinosylvin; Plumbagin; Plumbagine; Podophyllotoxin; Podophyllotoxone; Podorhizol beta-D-glucoside; Polyandrol; Polyphyllin I; Ponapensin; Ponciretin; Poncirin; Pongapinone A; Pongapinone B; Pongavilleanine; Praecansone B; Pretazettine; Pristimerin; Progallin A; Protocatechuic acid; Prunectin; Prunetol; Pseudobaptigenin; Psoralen; Psorospermin; Psychorubrin; Psychotrine; Ptaeroglycol; Puerarin; Pulegone; Pulsatillic acid; Pulverochromenol; Puromycin; Purpuracenin; Purpurediolin; Purpurenin; Quercetagetin 3,6,7,4′-tetramethyl ether; Quercetin; Quercetin 3,5,7,3′,4′-pentamethyl ether; Quercetin 3,7,3′,4′-tetramethyl ether; Quercetin 3-O-beta-D-galactopyranoside; Quercetin 3-O-beta-D-galactoside; Quercetin 3-O-beta-D-glucopyranoside; Quercetin 3-O-beta-D-glucoside; Quercetin 3-O-galactoside; Quercetin 3-beta-galactopyranoside; Quercetin 3-glucoside; Quercetin-3-O-beta-D-glucopyranoside; Quinic acid; Quinquangulin; Rabdophyllin G; Rabdosin C; Rabdoumbrosanin; Rapanone; Reissantins A; Reissantins B; Reissantins D; Reissantins E; Remangilones A; Remangilones C; Reserpic acid; Resiniferol 20-(4-hydroxy-3-methoxyphenylacetate) 9,13,14-ortho-phenylacetate; Resveratrol; Rhabarberone; Rhaponticin; Rhinacanthin A; Rhinacanthin B; Rhinacanthin C; Rhinacanthin D; Rhinacanthin G; Rhinacanthin H; Rhinacanthin I; Rhinacanthin K; Rhinacanthin M; Rhinacanthin N; Rhinacanthin Q; Rhinacanthone; Rhoeadine; Rhoifolin; Rhyncophylline; Ricin; Rocaglamide; Rohitukine; Rollidecin B; Rollinecins A; Rollinecins B; Rosmanol; Rosmarinic acid; Rotenolone; Rotenone; Rubriflorin A; Rubrofusarin; Rugosin D; Rutamarin; Rutin; S-allylcysteine; S-allylmercaptocysteine; Saikosaponin D; Saikosaponin a; Saikosaponin b2; Salutaridine; Salvileucalin B; Samaderine Z; Sanggenon C; Sanggenon D; Sanguinarine; Santol; Sapindoside B; Saponaretin; Saprosmine A; Saprosmine B; Sarsasapogenin; Sativanone; Schweinfurthin A; Schweinfurthin B; Scopoletin; Scullcapflavone II; Scutellarein; Secoantofine; Secofoveogline; Secundiflorol H; Sempervirine; Serpentine; Sesamin; Silicicolin; Silvestrol; Silymarin; Simalikalactone D; Sinensetin; Sitoindoside IX; Smilax saponin B; Sodium butyrate; Solamargine; Sophoraflavanone G; Sophorastilbene A; Sophoricol; Spathelia bischromene; Spathulenol; Spermatheridine; Spinasterol; Spinencin; Spirocurcasone; Sporidesmin; Squalene; Squamocin; Squamocin-D; Squamostatin-A; Squamostatin-B; Squamostatin-D; Squamostatin-E; Stigmasterol; Strychnine; Stylophorine; Styraxin; Subtrifloralactone A; Subtrifloralactone B; Subtrifloralactone C; Subtrifloralactone D; Subtrifloralactone E; Subtrifloralactone F; Subtrifloralactone G; Subtrifloralactone H; Subtrifloralactone I; Subtrifloralactone J; Subtrifloralactone K; Sulforaphane; Sulphurenic acid; Sumatrol; Swainsonine; Syriacusins A; T-Muurolol; T-cadinol; T-muurolol; Tabernamine; Tabersonine; Tagitinin C; Taiwaniaquinol A; Taiwaniaquinol B; Taiwaniaquinol C; Taiwaniaquinol D; Taiwaniaquinol E; Taiwaniaquinol F; Taiwaniaquinone A; Taiwaniaquinone D; Taiwaniaquinone E; Taiwaniaquinone F; Taiwaniaquinone G; Taiwaniaquinone H; Tamoxifen; Tangeretin; Tannic acid; Taxifolin; Taxiresinol; Taxol; Taxol B; Taxumairol Q; Teafuran 30A; Tectoridin; Tectorigenin; Tephrosin; Terpinen-4-ol; Tetra-o-methylisoscutellarein; Tetra-o-methylscutellarein; Tetrandrine; Thalicarpine; Thalmine; Thalsimine; Thalsine; Thymol; Thymoquinone; Tiliroside; Tithofolinolide; Tomatidenol; Tomatidine; Tonkinecin; Toosendanal; Toosendanin; Toxol; Toxyl angelate; Trans Farnesol; Trans-3-(3,4-dimethoxyphenyl)-4-[(E)-3,4-dimethoxystyryl]cyclohex-1-ene; Trans-Zeatin; Triacetylbalsaminol F; Tribenzoylbalsaminol F; Trichilin H; Trichothecin; Trichothecinol A; Trichothecolone; Tricin; Tuberculatin; Tubulosine; Tylophorine; Umbellatin; Uncarine E; Uncarinic acid A; Uncarinic acid B; Uncarinic acid C; Uncarinic acid D; Uncarinic acid E; Ursolic acid; Uvangoletin; Uvaretin; Uvaribonin; Uvaricin; Veraphenol; Verbenone; Vibsanins P; Vibsanins Q; Vibsanins R; Vibsanins S; Vibsanins T; Vibsanins V; Vibsanins W; Vinblastine; Vincamine; Vincristine; Vincristine sulfate; Viscidulin I; Viscidulin II; Viscidulin III; Viscosalactone B; Vismiaguianin A; Vismiaguianone A; Vismiaguianone B; Vismiaguianone C; Vismiaguianone D; Vismiaguianone E; Vismin; Vismione B; Vismione D; Vitexicarpin; Vitexin; Wedelolactone; Weisiensin A; Wighteone; Wikstroelides A; Wikstroelides D; Wikstroelides E; Wikstroelides I; Wikstroelides J; Withaferin A; Withanoside IV; Withaphysacarpin; Wogonin; Xanthatin; Xanthochymol; Xanthohumol; Xanthotoxol; Xanthoxylin; Xanthyletin; Xenognosin A; Xerophilusin A; Xerophilusin B; Xindongnin B; Yangambin; Yatein; Yunnanin A; Z-Guggulsterone; Zapotin; Zeaxanthin; Zerumin A; Zhankuic acid A; Zhankuic acid C; Zygadenine; [1aR-(1aalpha,3beta,3aalpha,5abeta,8aalpha,8bbeta,8calpha)]-Decahydro-3-hydroxy-3a,8c-dimethyl-6-methyleneoxireno[7,8]naphtho[1,2-b]furan-7(2H)-one; [3-O-beta-D-glucopyranosyl-(1->3)-alpha-L-rhamnopyranosyl-(1->2)-alpha-L-arabinopyranosyl echinocistycacid]; [3]-dehydroparadol; [6]-Gingerol; acacetin; acetylbetulinic acid; acetylursolic acid; actein; acuminatin; aglycon 14-hydroxyhypocretenolide; all-trans-(+)-Xanthophyll; all-trans-Lycopene; allo-Isopteropodine; alpha-L-arabinopyranoside; alpha-Mangostin; alpha-Naphthoflavone; alpha-Peltatin; alpha-Solanine; alpha-Spinasterol; alpha-Toxicarol; alpha-amyrin; alpha-cadinol; alpha-lapachone; alpha-santonin; alphitolic acid; annocatacin A; annocatacin B; apigenin-7-O-beta-D-glucopyranoside; arteglasin A; artocarpin; balanophonin; beta,beta-Dimethylacrylshikonin; beta-Lapachone; beta-Mangostin; beta-Peltatin A methyl ether; beta-carboline-1-propionic acid; beta-caryophyllene 4,5 alpha-oxide; beta-caryophyllene oxide; beta-phellandrene; beta-pinene; betulonic acid; bitucarpin A; borneol; bornyl acetate; calopocarpin; camphor; candenatenin A; candenatenin B; candenatenin C; candenatenin D; candidin; candidone; canin; canophyllic acid; canthin-6-one; canthin-6-one 9-O-beta-glucopyranoside; caulophyllogenin3-O-beta-Dglucopyranosyl-(1->3)-alpha-L-rhamnopyranosyl-(1->2)-alpha-L-arabinopyranosidic; chicoric acid; chlorogenic acid; chloroquine; chrysoeriol 7-O-beta-D-glucopyranoside; cimigenol 3-O-alpha-L-arabinopyranoside; cimiracemoside F; cimiracemoside G; cimiracemoside H; cis-3-O-p-Hydroxycinnamoyl Ursolic Acid; colubrinic acid; convallatoxin; convallatoxol; convalloside; corossolin; corossolone; coumestrol; d-(+)-Limonene; d-Berbamine; damsin; deacetoxymatricarin; deca-2,4 diene-4-hydrooxy-6-yn-1,4-olide; deca-4,6-diyn-2-(Z)-enoic ethyl ester; deca-4,6-diyn-2-(Z)-enoic methyl ester; dehydro-iso-alpha-lapachone; dimethylmatairesinol; dl-a-Tocopherol; echinacoside; encelin; ent-18-Hydroxytrachyloban-3-one; ent-kaur-16-en-13,19-diol; epi-alpha-Muurolol; erybraedin C; eupafolin; eurycomanone; eurylene; farinosin; forsythoside; gamma-Mangostin; gamma-caryophyllene; glucostrophanthidin; harunganin; helenalin; helenalin acetate; helioxanthin; herbacetin 3,8,4′-trimethyl ether; hexanorcucurbitacin F; hinokinin; inuloidin; iso-Dulcinol; isochaihulactone; isoneorautenol; isopseudopsoralen; isoverbascoside; kaempferide; kaempferol-3-methoxy-7-O-alpha-L-rhamnoside; kaempferol-3-methyl ether; 1-Epigallocatechin; lespedezaflavanone B; liriodendrin; liriodenine; longifolicin; longikaurin B; longilactone; luteolin 7-o-beta-D-glucopyranoside; meso-(rel 7S,8S,7'S,8'S)-3,4,3′,4′-tetrahydroxy-7,7′-epoxylignan; mulberrofuran B; mulberrofuran D; mulberrofuran L; mulberrofuran W; mulberrofuran X; mulberrofuran Y; mulberrofuran Z; naviculol; naviculyl caffeate; neoannonin; neorautenol; oleanonic acid; p-Coumaric acid; p-Methoxycinnamic acid ethyl ester; phaseollin; phenethylisothiocyanate; pseudoisopsoralen; purpurin; rel-(3S,4aR,10bR)-8-hydroxy-3-(4-hydroxyphenyl)-9-methoxy-4a,5,6,-10b-tetrahydro-3H-naphtho[2,1-b]pyran; ridentin; rocagloic acid; rollidecin A; rollinmusin; sanggenol M; sanggenon A; sanggenon B; sanggenon C; sanggenon D; sanggenon M; sanggenon O; savinin; sorocein D; sorocein F; strophanthidin; sulfuretin; taiwanin A; taiwanin C; taiwanin E; taxuspine; terpinolene; thiacremonone; toxicarioside M; trans-3-O-p-Hydroxycinnamoyl Ursolic Acid; trans-Phytol; tricyclene; tylocrebrine; tyloindicine F; tyloindicine G; tylophoridicine C; tylophoridicine F; ursonic acid; vibsanin C; vogelin C; vulgarin; xanthinin; xanthone V1; xanthopurpurin; xerophilusin F; zizyberenalic acid.
AANPI may be in the form of a pure substance isolated from a natural source, in the form of an extract, a dried extract, or in the form of a processed natral source, such as a powder and any other form in which it is contained.

In another embodiment, provided are the method when the non-covalent complex or solid dispersion or conjugate is combined with at least one additional active natutral pharmaceutical ingredient (AANPI) selected from: folate, glycyrrhizic acid, β-sitosterol-β-D-glucoside (Sito-G), asialoorosomucoid, and asialofetuin.

In another embodiment, provided are the method when the non-covalent complex or solid dispersion or conjugate is combined with combination of at least one of: radiation therapy; photodynamic therapy; photoimmunotherapy; surgery; chemotherapy, cytotoxic chemotherapy; Immunotherapy, immunotherapy with modified gamma delta T cells; hormonal therapy; oncolytic virus therapy; ketogenic diet+dexamethasone (Miriam Ferrer et al., Ketogenic diet promotes tumor ferroptosis but induces relative corticosterone deficiency that accelerates cachexia, Cell Metabolism, 2023); and the like.

### VI. PHARMACEUTICAL FORMULATIONS/COMPOSITIONS (BASED ON FORMULA I-III COMPOUNDS)

This section describes pharmaceutical formulations/compositions/pharmaceutical & medical dosage forms based on Formula I-III compounds which can be composed and prepared in various ways. The compounds of the present invention are formulated with common ballast materials and excipients, which are selected according to common practice. Tablets will contain excipients, glydants, fillers, binders, and the like. Aqueous formulations are prepared in sterile form, and if they are not intended to be administered orally, they are usually isotonic. All formulations optionally contain excipients, such as those described in Handbook of Pharmaceutical Excipients (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is usually between 7 and 10. In some embodiments of the invention, the pH of the formulations varies from about 2 to about 5, but is usually about 3 to 4.

Although the active ingredients may be administered separately, it may be preferable to present them as pharmaceutical compositions. Compositions of the invention, whether for veterinary or human use, include at least one active ingredient, as defined above, together with one or more acceptable ballast materials for it and, optionally, other therapeutic ingredients, in particular additional therapeutic ingredients, which are discussed herein. The ballast materials must be “acceptable” in the sense of being compatible with the other ingredients of the composition and physiologically harmless to the recipient.

The compositions include those suitable for the above-mentioned routes of administration. The compositions may be conveniently presented in unit dose form and may be prepared by any of the methods well known in the pharmaceutical art. The methods and compositions are generally described in Remington's Pharmaceutical Sciences (Mack Publishing Co., Easton, Pa.). Such methods involve the step of bringing into association the active ingredient with the ballast materials which constitutes one or more accessory ingredients. In general the formulations are prepared by uniformly and intimately bringing into association the active ingredient with liquid ballast materials or finely divided solid ballast materials or both, and then shaping the product if necessary.

Formulations of the present invention suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can be administered as a bolus, electuary or paste.

Tablets are made by compression or molding, optionally with one or more additional ingredients. Compressed tablets can be prepared by compressing in a suitable machine an active ingredient in a free-flowing form, such as a powder or granules, further mixed with a binder, lubricant, inert diluent, preservative, surface-active or dispersing agent. Moulded tablets can be made by molding in a suitable machine a mixture of a powdered active ingredient moistened with an inert liquid diluent. The tablets may additionally be coated with a shell or pellets and optionally formulated to allow slow or controlled release of the active ingredient therefrom.

For diseases of the eyes or other external tissues, such as the mouth and skin, the compositions are preferably applied in the form of an ointment or cream containing the active ingredient(s) in an amount, for example, from 0.075 to 20% wt. (including the active ingredient(s) in a range of 0.1% to 20% in 0.1% wt. increments, e.g., 0.6% wt., 0.7% wt., etc.), more preferably from 0.2 to 15% wt. and most preferably from 0.5 to 10% wt. When formulated as an ointment, the active ingredients may be used with a paraffin or water-miscible ointment base. As an alternative, the active ingredients may be formulated as a cream with an oil-in-water type cream base.

Optionally, the aqueous phase of the cream base may include, if desired, at least 30% wt. of a polyhydric alcohol, that is, an alcohol with two or more hydroxyl groups, such as propylene glycol, butane-1,3-diol, mannitol, sorbitol, glycerol, polyethylene glycol (including PEG 400) and mixtures thereof. Compositions for topical use may preferably include a compound that enhances absorption or penetration of the active ingredient through the skin or other affected areas. Among examples of such dermal penetration enhancers are dimethyl sulfoxide and related analogues.

The oil phase of emulsions of the present invention can be composed of known ingredients in a known manner. Although the phase may comprise an emulsifier alone (otherwise referred to as emulsifier), preferably it comprises a mixture of at least one emulsifier with a fat or oil or with a fat and oil. The hydrophilic emulsifier is preferably included together with a lipophilic emulsifier, acting as a stabilizer. Also preferably both oil and fat are included. Emulsifier(s) with or without stabilizer(s) together constitute the so-called emulsifying wax, and the wax together with oil and fat constitute the so-called emulsifying ointment base, which forms the oily dispersed phase of the cream compositions.

Emulsifiers and emulsion stabilizers appropriate for use in the formulation of the invention include Tween® 60, Span® 80, cetostearyl alcohol, benzyl alcohol, myristyl alcohol, glyceryl monostearate and sodium lauryl sulfate. Other emulsifiers and emulsion stabilizers appropriate for use in the formulation of the invention include Tween® 80.

The choice of suitable oils or fats for the formulation is determined by achieving the desired cosmetic properties. The cream may be a non-greasy, non-staining and washable product with a suitable consistency to avoid leakage from tubes or other containers. Straight or branched chain alkyl esters, mono- or dibasic, such as di-isoadipate, isocetyl stearate, propylene glycol coconut fatty acid diester, isopropyl myristate, decyl oleate, isopropyl palmitate, butyl stearate, 2-ethylhexyl palmitate or a mixture of branched chain esters known as Crodamol CAP, the latter three being preferred. They may be used alone or in combination depending on the desired properties. As an alternative, high melting point lipids such as white soft paraffin and/or liquid paraffin or other mineral oils are used.

According to the present invention, pharmaceutical compositions include a combination together with one or more pharmaceutically acceptable carriers or excipients and optionally other therapeutic agents. The pharmaceutical compositions containing the active ingredient may be in any form suitable for the intended route of administration. For oral administration, the compositions may be prepared, for example, tablets, trochets, lozenges, aqueous or oily suspensions, dispersible powders or granules, emulsions, hard or soft capsules, syrups or elixirs. Formulations intended for oral use may be made according to any method known in the art for making pharmaceutical compositions, and such compositions may contain one or more agents, including sweeteners, flavorings, colorings, and preservative agents, to provide a palatable preparation. Tablets containing the active ingredient in a mixture with a non-toxic pharmaceutically acceptable excipient that is suitable for tablet production are acceptable. Such excipients may be, for example, inert diluents such as calcium or sodium carbonate, lactose, calcium or sodium phosphate; granulating and disintegrating agents such as corn starch or alginic acid; binding agents such as starch, gelatin or acacia; and lubricating agents such as magnesium stearate, stearic acid or talc. Tablets may be uncoated or may be coated using known methods, including microencapsulation, to delay breakdown and adsorption in the gastrointestinal tract and thereby provide sustained action over an extended period. A time delayed material such as glyceryl monostearate or glyceryl dystearate alone or with a wax, for example, may be used.

Products for oral use can also be designed as hard gelatin capsules where the active ingredient is mixed with an inert solid diluent, such as calcium phosphate or kaolin, or as soft gelatin capsules where the active ingredient is mixed with water or an oil medium, such as peanut oil, liquid paraffin or olive oil.

Aqueous suspensions according to the invention contain active materials mixed with excipients suitable for making aqueous suspensions. Such excipients may include a suspending agent such as sodium carboxymethylcellulose, methylcellulose, hydroxypropyl methylcellulose, sodium alginate, polyvinylpyrrolidone, tragacanth gum and acacia gum, and dispersing or wetting agents such as naturally occurring phosphatide (such as lecithin), an alkylene oxide condensation product with a fatty acid (e.g., polyoxyethylene stearate), an ethylene oxide condensation product with a long-chain aliphatic alcohol (e.g., hepta dekaethylene acetanol), an ethylene oxide condensation product with a partial ester derived from a fatty acid and hexitol anhydride (e.g., polyoxyethylene sorbitan monoleate). The aqueous suspension can also contain one or more preservatives such as ethyl- or n-propyl p-hydroxy-benzoate, one or more colorants, one or more flavorings, and one or more sweeteners such as sucrose or saccharin. Other non-limiting examples of suspending agents include Cyclodextrin and Captisol.

Oil suspensions can be prepared by suspending the active ingredient in a vegetable oil such as peanut oil, olive oil, sesame oil or coconut oil, or in a mineral oil such as liquid paraffin. A thickening agent such as beeswax, solid paraffin or cetyl alcohol may be contained in the oral suspension. Sweeteners, such as those described above, and flavorings may be added to produce a palatable oral preparation. These compositions may be preserved by adding an antioxidant such as ascorbic acid.

Dispersible powders and granules of the invention, applicable for preparing an aqueous suspension by adding water, include the active ingredient in a mixture with a dispersing or wetting agent, a suspending agent and one or more preservatives. Suitable dispersing or wetting agents and suspending agents are examples of those disclosed above. Additional excipients, such as sweetening, loosening and coloring agents, may also be present.

The pharmaceutical compositions of the invention can also be presented as oil-in-water emulsions. The oil phase can be a vegetable oil such as olive or peanut oil, a mineral oil such as liquid paraffin, or a mixture thereof. Appropriate emulsifiers include gum of natural origin such as acacia gum and tragacanth gum, phosphatides of natural origin, esters or partial esters derived from fatty acids and hexitol anhydrides such as sorbitan monooleate, and condensation products of these partial esters with ethylene oxide such as polyoxyethylene sorbitan monooleate. The emulsion can also contain sweeteners and flavorings. Syrups and elixirs may contain sweetening agents such as glycerin, sorbitol, or sucrose. These formulations can also contain a demulgent, preservative, flavoring, or coloring agent.

The pharmaceutical compositions of the invention may be in the form of a sterile injectable preparation, such as a sterile injectable aqueous or oily suspension. This suspension may be prepared according to the prior art using suitable dispersing or wetting agents and suspending agents as mentioned above. The sterile injectable preparation may also be a sterile injectable solution or suspension in a non-toxic parenterally acceptable diluent or solvent, such as a solution in 1,3-butanediol or prepared as a lyophilized powder. Acceptable vehicles and solvents that may be used include water, Ringer's solution, and isotonic sodium chloride solution. In addition, sterile fixed oils are usually used as a solvent or suspension medium. Any tasteless fixed oil can be used for this purpose, including synthetic mono- or diglycerides. In addition, fatty acids, such as oleic acid, can also be used to prepare injectable preparations. Among the acceptable vehicles and solvents that can be used are water, isotonic Ringer's solution, isotonic sodium chloride solution and hypertonic sodium chloride solution.

The amount of active ingredient that can be combined with a carrier material to produce a single dosage form will vary depending on the recipient and the particular route of administration. For example, a time-release formulation intended for oral administration to humans may contain from about 1 to 1000 mg of the active ingredient combined with an appropriate and convenient amount of carrier material, which may be from about 5 to about 95% of the total amount of the composition (weight:weight). The pharmaceutical composition may be prepared so as to provide an easily measurable amount for administration. For example, an aqueous solution intended for intravenous infusion may contain from about 3 to about 500 μg of active ingredient per milliliter of solution to provide an infusion of a suitable volume at a rate of about 30 ml/hr.

Formulations suitable for topical administration to the eye also include eye drops in which the active ingredient is dissolved or suspended in a suitable ballast materials, especially in an aqueous solvent for the active ingredient. The active ingredient is preferably present in such compositions in a concentration of from 0.5 to 20%, more preferably from 0.5 to 10% and especially about 1.5% w/w.

Formulations suitable for topical use in the mouth include lozenges containing the active ingredient in a flavored base, usually sucrose and acacia or tragacanth; lozenges containing the active ingredient in an inert base such as gelatin and glycerin or sucrose and acacia; and mouthwashes containing the active ingredient in a suitable liquid carrier.

Formulations for rectal administration may be in the form of a suppository with a suitable base including, for example, cocoa butter or salicylate.

Formulations suitable for intrapulmonary or nasal administration have particle sizes, for example, in the range of 0.5 to 500 microns, such as 0.5, 1, 30, 35, etc., which are administered by rapid inhalation through the nasal passage or inhalation through the mouth to reach the alveolar sacs. Suitable compositions include aqueous or oily solutions of the active ingredient. Formulations suitable for administration as an aerosol or dry powder may be prepared according to conventional methods and may be delivered together with other therapeutic agents, such as compounds previously used to treat or prevent “diseases of the invention”, as described below.

Formulations suitable for vaginal administration may be in the form of pessaries, tampons, creams, gels, pastes, foams or sprays containing in addition to the active ingredient such carriers as are known in the art to be suitable.

Formulations suitable for parenteral administration include aqueous and non-aqueous sterile solutions for injection, which may contain antioxidants, buffers, bacteriostatics and solvents that make the formulation isotonic with the blood of the intended recipient; and aqueous and non-aqueous sterile suspensions, which may include suspending and thickening agents.

The formulations are presented in single or multi-dose containers, such as sealed ampoules and vials, and can be stored in a freeze-dried (lyophilized) state requiring only the addition of a sterile liquid carrier, such as water for injection, immediately prior to use. Extemporaneous injectable solutions and suspensions are made from sterile powders, pellets, and tablets described earlier. Preferred single-dose preparations are preparations containing a daily dose or a single daily subdose, as described above, or an appropriate fraction of the active ingredient.

It is important to note that in addition to the ingredients especially mentioned above, the compositions of the present invention may include other agents common in the art, taking into account the type of composition in question, for example, compositions suitable for oral use can include flavorings.

The invention also provides veterinary compositions comprising at least one active ingredient, as defined above, together with a veterinary carrier for it.

Veterinary carriers are materials useful for administering the composition and may be solids, liquids, or gases that are inert or acceptable in veterinary medicine and compatible with the active ingredient. These veterinary compositions may be administered orally, parenterally, or by any other desired route.

Compounds of the invention are used to make controlled-release pharmaceutical compositions containing as an active ingredient one or more compounds of the invention (“controlled-release compositions”) in which the release of the active ingredient is controlled and regulated to provide less frequent dosing or to improve the pharmacokinetic or toxicity profile of that active ingredient.

In another embodiment, the method comprises administering a therapeutically effective amount of a formulation in form of solution/emulsion/suspension comprising:
a) a first pharmaceutical agent comprising a compound of Formula I-III, and
b) at least one of the following components:
   (1) oil phase containing vegetable or/and animal fats;
   (2) one or more surfactants;
   (3) one or more solvents;
   (4) one or more gelling agents.

Formulation can be a true solution or colloidal solution, emulsion, solid or liquid dispersion, suspension, emulsion-suspension form, gas cocktail, foam, sol, gel, paste, their mixture. Formulation can be prepared by mixing, homogenizing, dispersing, and emulsifying the constituent components.

In another embodiment, the method comprises administering a therapeutically effective amount of a formulation comprising:
(a) oil phase containing vegetable or/and animal fats or/and lecithin 1-99.999% by weight (b) a compound of Formula I-III or/and API 0.001-99% by weight
(c) (optional) one or more surfactants 0-70% by weight (physiologically acceptable surfactant selected from the surfactants listed in the present invention).
(d) (optional) one or more solvents 0-99.9% by weight (physiologically acceptable solvent selected from the solvents listed in the present invention).
(e) (optional) one or more gelling agents (0-99.9% by weight) include (but are not limited to): alginate, pectin, carrageenan, gellan, gelatin, agar, modified starch, methyl cellulose, and hydroxypropylmethylcellulose.

In another embodiment, the method comprises administering a therapeutically effective amount of a complex pharmaceutical agent/pharmaceutical formulation/composition comprising:
(a) (optional) oil phase containing vegetable or/and animal fats or/and lecithin 0-99.999% by weight
(b) a compound of Formula I-III or/and API 0.001-99% by weight
(c) (optional) one or more surfactants 0-70% by weight (physiologically acceptable surfactant selected from the surfactants listed in the present invention).
(d) one or more solvents 5-99.9% by weight (physiologically acceptable solvent selected from the solvents listed in the present invention).
(e) (optional) one or more gelling agents (0-99.9% by weight) include (but are not limited to): alginate, pectin, carrageenan, gellan, gelatin, agar, modified starch, methyl cellulose, and hydroxypropylmethylcellulose.

In another embodiment, the method comprises administering a therapeutically effective amount of a complex pharmaceutical agent/pharmaceutical formulation/composition comprising:
(a) oil phase containing vegetable or/and animal fats or/and lecithin 1-99.999% by weight
(b) a compound of Formula I-III or/and API 0.001-99% by weight
(c) (optional) one or more surfactants 0-70% by weight (physiologically acceptable surfactant selected from the surfactants listed in the present invention).
(d) one or more solvents 1-99.9% by weight (physiologically acceptable solvent selected from the solvents listed in the present invention).
(e) (optional) one or more gelling agents (0-99.9% by weight) include (but are not limited to): alginate, pectin, carrageenan, gellan, gelatin, agar, modified starch, methyl cellulose, and hydroxypropylmethylcellulose.

In another embodiment, the method comprises administering a therapeutically effective amount of a complex pharmaceutical agent/pharmaceutical formulation/composition comprising:
(a) (optional) oil phase containing vegetable or/and animal fats or/and lecithin 0-99.999% by weight
(b) a compound of Formula I-III or/and API 0.001-99% by weight
(c) one or more surfactants 0.05-70% by weight (physiologically acceptable surfactant selected from the surfactants listed in the present invention).
(d) (optional) one or more solvents 0-99.9% by weight (physiologically acceptable solvent selected from the solvents listed in the present invention).
(e) (optional) one or more gelling agents (0-99.9% by weight) include (but are not limited to): alginate, pectin, carrageenan, gellan, gelatin, agar, modified starch, methyl cellulose, and hydroxypropylmethylcellulose.

The active compounds of Formula I-III can be administered/can be in form of foods, food additives, edible soluble films, drinks, medicinal agents, and feeds for domestic and wild animals. The drinks may be non-alcohol drinks or alcohol drinks. Examples of non-alcohol drinks include carbonated drinks, noncarbonated drinks (such as fruit juice, and nectar), soft drinks, sports drinks, tea, coffee, and hot chocolate. The alcohol drinks may be in the form of, for example, beer, low-malt beer, third-category beer, sake, wine, champagne, liqueur, or medicated liquor. For use as a food material or food additive (e.g., human food or animal food, such as a dog or cat food or feed for poultry, cows, or pigs), the active compound may be in the form of, for example, a tablet, a capsule formulation, a solid agent (such as a powder and a granule) dissolved in drinks, a semi-solid such as jelly, a liquid (such as drinking water), and a high-concentration solution diluted before use. Optional components, such as vitamins, carbohydrates, dyes, and flavoring agents commonly added to food may be appropriately mixed. The food may be given in any form, including a liquid and a solid.

In another embodiment, the method comprises administering a therapeutically effective amount of a complex pharmaceutical agent in form of food product or beverage, or dietary supplement.

In another embodiment, provided are the method when additional active pharmaceutical ingredient (AAPI) or additional active natutral pharmaceutical ingredient (AANPI) is formulated in form of non-covalent complex or molecular/solid dispersion or conjugate according to the methods and techniques of the present invention (i.e., when instead of API being used AAPI or AANPI).

In another embodiment, provided are the method when additional active pharmaceutical ingredient (AAPI) or additional active natutral pharmaceutical ingredient (AANPI) are co-processed with the API to form their joint non-covalent complex or solid dispersion or conjugate.

In another embodiment, provided are the method when active pharmaceutical ingredient (API) and/or additional active pharmaceutical ingredient (AAPI) and/or additional active natutral pharmaceutical ingredient (AANPI) is formulated in form of:
A) a stabilized aqueous formulation which comprises API and/or AAPI and/or AANPI, solvent, surface active agent and cosolvent or a stabilized formulation which comprises API and/or AAPI and/or AANPI solutions in fat/oil or API and/or AAPI and/or AANPI emulsion.
B) The method of claim 9, 10 wherein the compound is a compound of Formula IV in the form of solution/emulsion/suspension comprising:
   a) a first pharmaceutical composition comprising a compound of Formula I-III, and
   b) at least one of the following components:
      (1) oil phase containing vegetable or/and animal fats;
      (2) one or more surfactants;
      (3) one or more solvents;
      (4) one or more gelling agents;
C) nanoparticles, microparticles, nanocomposites, microcomposites, composites, polymeric nanoparticles (PNPs), liposomes, freeze-dried liposomes, micelles, polymeric micelles, niosomes, solid lipid nanoparticles (SLNs), nanostructured lipid carriers (NLCs), nanoemulsions, microemulsions, emulsions, self-emulsifying (nano or micro) drug delivery systems (SNEDDS, SMEDDS, SEDDS), nanocrystals, co-crystals, mesoporous silica nanoparticles (MSNs), dendrimers, ultradispersions, solid dispersions, solid amorphous dispersions, noncovalent complexes, complexes with polymer, complexes with biopolymer, host-guest complexes, inclusion complexes, solutions, solutions in fat/oil, solutions with cosolvent, solutions with surface active agent/surfactant, solutions with solubilizing agent, polymorphes, lipid-based systems, lipid-based formulations, lipid-based drug delivery systems (LBDDS), gels, colloids, sols, and the like, or in any form described in the scientific and patent literature for example;
D) a food product or beverage, or dietary supplement.
or/and API, AAPI, AANPI can be obtained using methods of: A) Reduction Particle size: a) Micronization; b) Nanosuspension; B) Modification of the crystal habit; C) Solid dispersions: a) Eutectic mixtures; b) Solid solutions; c) Amorphous solid solutions; d) Glass solutions and glass suspension; e) Cryogenic techniques; A) Change of pH; B) Use of buffer; C) Derivatization; D) Complexation; E) Salt formation; A) Supercritical fluid process; B) Use of adjuvant like surfactant, solubilizers, cosolvency, hydrotropy, and novel excipients.

### VII. ROUTES OF ADMINISTRATION

The compounds of Formula I-IV described herein may be administered (e.g., orally) in the form of a solid or liquid dosage form. In both, the compounds may be coated in a material to protect them from the action of acids and other natural conditions which may inactivate the compounds. The compounds may be formulated as aqueous solutions, liquid dispersions, (ingestible) tablets, buccal tablets, troches, capsules, elixirs, powders, granules, ointments, adhesive skin patches, sprays, suspensions, syrups, and wafers. The dosage forms may include pharmaceutically acceptable excipients, diluents, and/or carriers known in the art, such as binders, disintegrating agents, emulsifiers, lubricants, flavorants, antioxidants, and preservatives. Liquid dosage forms may include diluents such as saline or an aqueous buffer.

Any route of administration Formula I-IV may be selected for use in the methods described herein. For instance, the route of administration may be selected from oral, nasal, topical, buccal, rectal, vaginal, ophthalmic, subcutaneous, intramuscular, intraperitoneal, epidural, intravenous, intraarterial, intratumoral, spinal, intrathecal, intra-articular, intra-arterial, sub-arachnoid, sublingual, oral mucosal, pulmonary, bronchial, lymphatic, intra-uterine, subcutaneous, intratumor, integrated on an implantable device, intradural, intracortical, intradermal, dermal, epidermal, transdermal, vaginal, rectal, ocular (for examples through the conjunctiva), intraocular, uretal, and parenteral. An advantage of the compounds of this invention is that they are orally bioavailable and can be dosed orally. A preferred route of administration is oral.

In the methods of the present invention for treating “diseases of the invention”, compounds of the present invention may be administered at any time to a person who may be in contact with people suffering from “diseases of the invention” or already suffering from “diseases of the invention”. In some embodiments, the compounds of the present invention can be administered prophylactically to people in contact with people suffering from “diseases of the invention”. In some embodiments of the invention, the compounds of the present invention can be administered to people who test positive for “diseases of the invention” but do not yet have symptoms of “diseases of the invention”. In some embodiments of the invention, the compounds of the present invention can be administered to people after symptoms of “diseases of the invention” appear.

The effective dose of the active ingredient depends at least on the nature of the disease being treated, the toxicity, whether the compound is used prophylactically (lower doses) or against an active “diseases of the invention”, the method of delivery and the pharmaceutical formulation, and is determined by the physician using routine dose escalation studies. It can be expected to range from about 0.0001 to about 100 mg/kg body weight per day; typically from about 0.01 to about 10 mg/kg body weight per day; more typically from about 0.01 to about 5 mg/kg body weight per day; most typically from about 0.2 to about 1 mg/kg body weight per day. For example, a daily candidate dose for an adult with a body weight of about 70 kg would be from about 1 mg to 1000 mg, preferably from 5 mg to 500 mg, and may take the form of a single or multiple dose. All dosages are given for an equivalent amount of pure API.

The effective dose of the compound of the present invention for treating “diseases of the invention” may depend on whether it is used prophylactically or to treat a person already suffering from “diseases of the invention”. Moreover, the dose may depend on whether the person suffering from “diseases of the invention” is not yet showing symptoms or is already showing symptoms of “diseases of the invention”. People who test positive for “diseases of the invention” and people with symptoms of “diseases of the invention” may require higher doses for treatment compared to people receiving preventive treatment.

Any suitable time period for administration of the compounds of the present invention is contemplated. For example, the administration may be from 1 day to 100 days, including 2, 3, 4, 5, 6, 7, 8, 9, 10, 15, 20, 25, 30, 35, 40, 50, 60, 70, 80 or 90 days. Admission may also last from 1 week to 15 weeks, including 2, 3, 4, 5, 6, 7, 8, 9, 10, 11, 12, 13 or 14 weeks. Longer periods of administration are also suggested. The timing of administration may depend on whether the compound is administered prophylactically or to treat a person suffering from “diseases of the invention”. For example, prophylactic administration may be for a period of time while the person is in constant contact with other people suffering from “diseases of the invention” and for a suitable period of time after the last contact with the person suffering from “diseases of the invention”. For people already suffering from “diseases of the invention”, the period of use may be any length of time necessary to treat the patient and a suitable period of time after a negative test for “diseases of the invention” to ensure that “diseases of the invention” will not return.

In another embodiment, provided are the method when non-covalent complexes or molecular/solid dispersions or conjugates are used as anticancer pharmaceutical agent, antitumor pharmaceutical agent, antineoplastic pharmaceutical agent, chemotherapeutic agent, or agent that is inductor, inhibitor or modulator of:
1) cell death in cancer cell lines;
2) cell death in senescent cell lines;
3) immunogenic cell death;
4) PAKI-mediated cytostatic autophagy;
5) caspase-dependent apoptosis;
6) WNT-T cell factor (TCF), Hippo and Akt/mTOR pathways;
7) growth and proliferation of cancer cells;
8) cancer stem-like cells.

In another embodiment, provided are the method when non-covalent complexes or molecular/solid dispersions or conjugates (alone or in combination with AAPI, AANPI) may be used for a wide range of active agents of the group of ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha 2-adrenergic agonists, analgesics, antipyretics and anti-inflammatory agents, androgens, anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretics, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinsons agents, antiplatelet agents, antiprogestins, antithyroid agents, anti-tussives, antiviral agents, a typical antidepressants, azaspirodecanediones, barbituates, benzodiazepines, benzothiadiazides, beta-adrenergic agonists, beta-adrenergic antagonists, selective beta 1-adrenergic antagonists, selective beta 2-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, haematopoietic agents, histamines, histamine antagonists, 5-hydroxytryptamine antagonists, drugs for the treatment of hyperlipoproteinemia, hypnotics and sedatives, immunosuppressive agents, laxatives, methylxanthines, monoamine oxidase inhibitors, neuromuscular blocking agents, organic nitrates, opiod analgesics and antagonists, pancreatic enzymes, pheno thiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins, hormones, steroids, nucleic acids, antibodies, enzymes, chemoprotective agents and radioprotecting agents

and
Abortifacient, Acaricidal, Allelopathic, Allergenic, Amnestic, Analgesic, Anesthetic, Anthelmintic, Antiallergic, Antiamebic, Antianemic, Anti-anxiety, Antiarrhythmic, Antiarthritic, Antiasthmatic, Antibacterial, Anticancer, Anticholinergic, Anticholinesterase, Anticonvulsant, Antidementic, Antidepressant, Antidermatitic, Antidiabetic, Antidiarrheic, Antidote, Antiedemic, Antiemetic, Antifertility, Antifungal, Antigout, Antihepatitic, Antihepatotoxic, Anti-HIV, Anti-HSV, Antihyperlipidemic, Antihypertensive, Anti-inflammatory, Antileishmanial, Antileprotic, Antimalarial, Antimicrobial, Antimigraine, Anti-muscle regidity, Antimutagenic, Antimyasthenic, Antineoplastic, Antioxidant, Antiparkinson, Antiprotozoal, Antipsychotic, Antipyretic, Antiseptic, Antispasmodic, Antithrombotic, Anti-tremor, Antitrypanosomal, Antituberculotic, Antitumor, Antitussive, Antiulcerogenic, Antiviral, Anxiogenic, Attractant, Biomarker, Cardiotonic, Carminative, Choleretic, CNS stimulant, Convulsant, Cytotoxic, Defense, Dental, Depilatory, Dermatitic, Diaphoretic, Diuretic, Edematous, Emetic, Emulsifying agent, Enhance flowering, Enhance fruiting, Enhance germination, Enhance leaf growth, Enhance plant growth, Enhance plant growth, Enhance root growth, Enhance stem growth, Essential amino acid, Expectorant, Feeding attractant, Feeding deterrent, Flavor, Genotoxic, Hallucinogenic, Hemolytic, Hemostatic, Hepatotoxic, Herbicidal, Hormonal, Hypnotic, Immunomodulative, Immunostimulant, Immunosuppressant, Induce tremor, Inhibit CYP, Inhibit flowering, Inhibit fruiting, Inhibit germination, Inhibit leaf growth, Inhibit plant growth, Inhibit root growth, Inhibit spore germination, Inhibit stem growth, Insecticidal, Irritant, Laxative, Molluscicidal, Muscle relaxant, Mutagenic, Narcotic, Nematocidal, Neurotoxic, Nonessential amino acid, Nucleic acid, Nutrient, Odor, Other cardiovascular agent, Other digestive organ agent, Other genitourinary agent, Other health agent, Other nervous system agent, Other respiratory tract agent, Oviposition attractant, Oviposition deterrent, Oxytocic, Pediculicidal, Phototoxic, Phytoalexin, Phytotoxic, Pigment, Piscicidal, Pneumotoxic, Pollinator attractant, Psychotomimetic, Repellent, Sedative, Sex attractant, Solvent, Stomachic, Teratogenic, Tonic, Toxic, Tumorigenic, UV shield, Vitamin agent.
and the like, alone or in combination. Although extensive, this list is not intended to be comprehensive.

In another embodiment, provided are the method when the dosage of the non-covalent complex or molecular/solid dispersion or conjugate exceeds EC 5 (5% of maximal effective concentration for API) for the respective cancer cell line.

In another embodiment, provided are the method when the dosage of the non-covalent complex or molecular/solid dispersion or conjugate exceeds EC 50 (half maximal effective concentration for API) for the cancer cell line.

In another embodiment, provided are the method the dosage of the non-covalent complex or molecular/solid dispersion or conjugate exceeds EC 90 (90% of maximal effective concentration for API) for the cancer cell line.

In another embodiment, provided are the method the mass ratio of API, AAPI, AANPI to polymer or carrier is from 1:1 to 1:10000.

Provided are the method of treatment of the cancer and/or method of manufacture a pharmaceutical dosage form to treat disease in a human or a mammal or an animal or a bird by administering a therapeutically effective amount of the molecular/solid dispersion or the noncovalent complex or conjugate to a human or a mammal or an animal or a bird in need thereof wherein the “carrier (host)” molecule of the molecular/solid dispersion, “carrier (host)” molecular part of non-covalent complex or conjugate interacts with the biological membranes of the organism in such a way that the transfer of molecules, ions or metabolites of API, AAPI, AANPI across this biological membrane is improved. Thus, the scope of the invention includes molecular/solid dispersions and non-covalent, inclusion complexes and conjugates with special permeability properties (highly permeable solid dispersions and non-covalent complexes and conjugates).

In another embodiment, provided are the complex, the solid dispersion, the conjugate has at least one of the altered properties compared to pure API, AAPI, AANPI:
1) The non-covalent complex, the solid dispersion, the conjugate has a higher bioavailability than pure API, AAPI, AANPI;
2) The non-covalent complex, the solid dispersion, the conjugate has a higher permeability than pure API, AAPI, AANPI;
3) The non-covalent complex, the solid dispersion, the conjugate has a lower toxicity or cytotoxicity than pure API, AAPI, AANPI;
4) The non-covalent complex, the solid dispersion, the conjugate has a different pharmokinetic parameters than API, AAPI, AANPI;
5) The non-covalent complex, the solid dispersion, the conjugate has a improved solubility than API, AAPI, AANPI.

In another embodiment, provided are the method of preparation of non-covalent complex, solid dispersion, or conjugate is selected from listed in the present invention.

In another embodiment, provided are the carrier chemically modified. I.e. carrier is a polymer, biopolymer or synthetic polymer, or methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified, esters and ethers, salts, and any other chemical derivatives thereof.

In another embodiment, provided are the complex, the solid dispersion, the conjugate use for directing a therapeutic agent to a selected cell target in a subject.

In another embodiment, provided are the methods wherein the therapeutic agent is covalently linked to the carrier.

In another embodiment, provided are the methods wherein the carrier has a plurality of carboxymethyl functional groups suitable for covalent linkage to the therapeutic agent.

In another embodiment, provided are the methods wherein the carrier is covalently linked to the therapeutic agent by a phosphoramidate linkage.

In another embodiment, provided are the methods wherein the carrier is covalently linked to the therapeutic agent by a ethylenediamine linkage.

In another embodiment, provided are the methods wherein non-covalent complex or solid dispersion or conjugate are used for the treatment of any disease.

In another embodiment, provided are the methods wherein non-covalent complex or solid dispersion or conjugate are used for the treatment of any disease listed in ICD 10.

In another embodiment, provided are the methods wherein non-covalent complex or solid dispersion or conjugate are used for the treatment of any disease listed in ICD 11.

In another embodiment, provided are the methods wherein non-covalent complex or solid dispersion or conjugate are used for the treatment of any disease listed in DSM-5.

In another embodiment, provided are the methods wherein non-covalent complex or solid dispersion or conjugate are used for the treatment of any disease listed in DSM-6.

### VIII. EXAMPLES

### A. Preparation of Compounds

### Example A.1

Ivermectin of pharmaceutical grade, CAS Number 70288-86-7, was purchased from Aecochem corp., Xiamen, China. Apple pectin was purchased from Ronas Chemicals Ind. Co., Ltd., Chengdu, China. Arabinogalactan, and all other chemicals were of analytical grade and used without further purification.

Method 1 was used to obtain compounds of Formula II. The roller ball mill was used to carry out mechanochemical synthesis/mechanoactivation process to form solid dispersions. Processing mode: rotational speed of the drive—400 rpm, rotational speed of the grinding jar—100 rpm, grinding jar volume—2000 ml, grinding media—steel balls (diameter 12 mm, 800 g/load), processing time predominantly ranging from 2 to 24 h and can be reduced by increasing the speed of rotation or increased by decreasing the speed of rotation. Samples are loose powders (particle size from 0.5-10 microns). Compounds and results of obtaining bioavailable solid dispersions of Formula I are shown in Table 1. Method 1 indicates that the grinding process can result in particle sizes primarily from 0.5 to 100 microns.

TABLE 1
Compounds of Formula I obtained in Example 1.

| No of | | | | | | |
|---|---|---|---|---|---|---|
| Formula I | | | API | Host | | |
| compound | API | Host substance | weight, g | substanceweight, g | Ratio | Note |
| 1 | Ivermectin | polyvinylpyrrolidone | 30 | 300 | 1:10 | |
| 2 | Ivermectin | apple pectin | 30 | 150 | 1:5 | |
| 3 | Ivermectin | laminaria | 30 | 300 | 1:10 | forms a gel with water |
| 4 | Ivermectin | SiO2 | 50 | 100 | 1:2 | silicon dioxide precipitates |
| | | | | | | when dissolved |
| 5 | Ivermectin | hydroxyethyl starch | 50 | 500 | 1:10 | forms clumps on air |
| 6 | Ivermectin | carboxymethylcellulose sodium salt | 50 | 150 | 1:3 | It takes a long time to dissolve |
| 7 | Ivermectin | fucus | 50 | 350 | 1:7 | forms a gel with water |
| 8 | Ivermectin | arabinogalactan | 50 | 250 | 1:5 | |
| 9 | Ivermectin | gum arabic | 50 | 250 | 1:5 | |
| 10 | Abamectin | arabinogalactan | 50 | 250 | 1:5 | |
| 11 | Moxidectin | gum arabic | 50 | 250 | 1:5 | |
| 12 | Niclosamide | arabinogalactan | 50 | 250 | 1:5 | |
| 13 | nitazoxanide | arabinogalactan | 50 | 250 | 1:5 | |
| 14 | mebendazol | gum arabic | 50 | 250 | 1:5 | |
| 15 | fenbendazol | gum arabic | 50 | 250 | 1:5 | |

### Example A.2

Method 10 was used to obtain compounds of Formula II. Acetone or ethyl alcohol (rectified 96%) was used as organic solvents: 40-70 parts of the solvent per 100 parts of the polymer and avermectin mixture taken in gram-equivalent quantities. The named solvents dissolve the polymer host. After thoroughly stirring in a mortar the polymer with avermectin moistened with solvent, a dough-like mass is formed, which is dried with evaporation of the solvent. Ball mills or other grinders can be used for grinding the dried polymer mass, and the particle size of the polymer product should not exceed 5-9 microns. Compositions and results of obtaining bioavailable solid dispersions with Formula Hare shown in Table 2. The process of grinding the solid dispersion after obtaining it does not affect its properties, since the change of substance has already occurred at the molecular level, the obtained solid dispersion can be used without further grinding.

TABLE 2
Compounds of Formula I obtained in Example 2.

| No of | | | | | | |
|---|---|---|---|---|---|---|
| Formula I | | | | Host | | |
| compound | Avermectin | Host substance | Avermectinweight, g | substanceweight, g | Ratio | Solvent |
| 1 | Ivermectin | polyvinylpyrrolidone | 3 | 15 | 1:5 | ethanol |
| 2 | Ivermectin | apple pectin | 2 | 10 | 1:5 | ethanol |
| 3 | Ivermectin | arabinogalactan | 2 | 10 | 1:5 | ethanol |
| 4 | Ivermectin | PVA-PEG co-polymer | 2 | 20 | 1:10 | acetone:ethanol 2:1 |

### Example A.3

Method 6 was used to obtain compounds of Formula 1. Ivermectin and PVA-polypropylene glycol graft co-polymers were used to create a solid dispersion. Hot stage extrusion was performed with a co-rotating, fully intermeshing conical mini twin screw extruder. The temperature was set at 195° C., the screw rate varied from 90 to 105 rpm. A load of 7.5 g per run was fed manually; after feeding the internal circulation time was 5 min. The extrudates were collected after cooling at ambient temperature on a conveyer belt. Extruded samples were subsequently milled for 4 min with a laboratory mill and sieved to exclude particles >400 pm.

### B. Bioavailability, Permeability and Solubility Studies

### Example B.1. Study of API Concentration in the Blood Plasma of Cattle

Free ivermectin substance was processed in planetary mill, the parameters were as follows: weight of the material—5 g, grinding jar volume—200 mL, grinding media—steel balls (diameter 8 mm, 90.0 g load), processing time—20 minutes. Compound of Formula I was treated according to Example 1 No21.

Cattle were divided into 2 groups of 1 animals each. Free Ivermectin (IVM) and compound of Formula I were orally administered to starving cattle. Blood samples were withdrawn from the jugular vein at appropriate time intervals after administration (0, 4, 20 h).

Ivermectin concentrations in blood plasma were analyzed according to the method Na-Bangchang, K., High-performance liquid chromatographic method for the determination of ivermectin in plasma, Southeast Asian J Trop Med Public Health, 2006 September, 37(5):848-58, PMID: 17333725.

The internal standard (moxidectin) was separated from ivermectin on a Hypersil Gold C18 column(150×4.6 mm, 5 m particle size), with retention time of 3.7 and 7.0 minutes, respectively. Fluorescence detection was set at an excitation and emission wavelength of 365 and 475 nm, respectively. The mobile phase consisted of acetonitrile, methanol and distilled water (50:45:5,v/v/v), running through the column at a flow rate of 1.5 ml/minute. The chromatographic analysis was operated at 25° C. Sample preparation (100 μl plasma) was done by a single step protein precipitation with acetonitrile, followed by derivatization with 100 μl of N-methylimidazolesolution in acetonitrile (1:1, v/v) and 150 μl of trifluoroacetic anhydrous solution in acetonitrile(1:2, v/v). Calibration curve over the concentration range of 20-8,000 ng/ml plasma was linear with correlation coefficient better than 0.995.

All the blood samples were centrifuged at 3000 rpm for 15 min to obtain serum. Cattle plasma samples (100 μL each) were placed in 1.5 mL Eppendorf tubes, and then the samples underwent protein precipitation. The samples were vortexed for 1 min and centrifuged at 13,400 rpm for 10 min 20 μL of the supernatant was injected into the HPLC.

Pharmacokinetic parameters of Ivermectin and compound of Formula I are shown in FIG. 1 and in Table 3. The results provide evidence of a substantial increase in the bioavailability and permeability of compound of Formula I. Oral administration of formula I 600 μg/kg IVM results in an excess of EC 50 2.4 μM for SARS-CoV-2.

TABLE 3
Pharmacokinetic parameters (ivermectin concentrartion) after
oral administrationof Ivermectin and compound of Formula I.

| | | compound of Formula I |
|---|---|---|
| | | of enhanced |
| | | bioavailability and |
| | IVM, 600 | permeability, 600 μg/kg |
| | μg/kg | (per pure IVM) |
| C1, 0 h, ng/ml | 0 ± 0.15 | 0 ± 0.15 |
| C2, 4 h, ng/ml | 307.3 ± 46.1 | 937.6 ± 140.6 |
| C3, 20 h, ng/ml | 104.4 ± 15.6 | 90.8 ± 13.6 |
| Cmax in plasma, μM | 0.35 ± 0.05 | 1.07 ± 0.16 |
| Cmax in lung, μM | 0.93 ± 0.14 | 2.86 ± 0.43 |
| calculated by | | |
| plasma-lung ratio 2.67 | | |

### Example B.2. Permeability of Solid Dispersion Aqueous Solution on the Caco-2 Cell Line

The Caco-2 cell line is a standard in vivo model for evaluating the permeability of drugs through the gastrointestinal wall in vivo. These cells retain almost complete morpho-functional properties inherent to intestinal epithelial cells, including the expression of multidrug resistance proteins such as P-glycoprotein. As a rule, the data obtained in such experiments have a good correlation with the bioavailability of drugs in living organisms.

Caco-2 cells were cultured in a standard way in Dulbecco's medium with the addition of 10% thermally inactivated bovine serum (FBS, USA). The permeabilization study through the Caco-2 cell monolayer was performed according to standard procedures. Transwell 12-well plates with a 0.4 μm polyester membrane (Corning, USA) were used, and cells were seeded at a density of 1×10⁵ cells per well. Before the experiment, cells were grown in IMDM medium supplemented with 10% FBS. The medium was changed every two days. The integrity of the cell monolayer was assessed by transepithelial electrical resistance (TEER) using Millicell ERS-2 (Millipore, USA) at the beginning and end of the experiment. The substance under study (compound of Formula I was treated according to Example 1 No 2) was dissolved in Hanks' balanced salt solution (HBSS) at concentrations of 3 μM (per pure IVM). Free Ivermectin was similarly dissolved in Hanks' balanced salt solution at concentrations of 3 μM. The test solutions were added to the top of the well, and the bottom of the well was filled with pure HBSS. The plates were then incubated at 37.5° C. and agitated (100 rpm) on an orbital shaker. Samples of 100 μL were taken from the bottom of the well at 20, 40, 60, 80, 100, and 120 min. After sampling, the required volume was immediately replenished with fresh HBSS solution. The concentration of substances was determined by high-performance liquid chromatography on Agilent 1200 HPLC with UV detector. The chromatographic analysis was performed on a reversed-phase column. The permeability coefficient was calculated according to a formula published in the literature. Thus, solid dispersion aqueous solution have better permeability than pure ivermectin.

TABLE 7
Permeability of IVM noncovalent complex through a monolayer
of Caco-2 cells compared to pure IVM at a similar point

| | | Concentrartion of IVM (for |
|---|---|---|
| | | compound of Formula I |
| | Concentrartion of IVM | of enhanced bioavailability |
| Time, min | (for pure IVM), μg/ml | and permeability), μg/ml |
| 20 | 0.05 | 0.1 |
| 40 | 0.09 | 0.19 |
| 60 | 0.15 | 0.31 |
| 80 | 0.19 | 0.43 |
| 100 | 0.21 | 0.66 |
| 120 | 0.25 | 0.8 |

### D. Antiviral Studies

### COVID-19 Patient Study M1: Blood Oxygen Saturation Levels

Blood oxygen saturation levels were measured in two symptomatic COVID-19 patients, one male (patient #1) and one female (patient #2), both testing positive for SARS-CoV-2. Patient #1 received an oral dose of 600 μg/kg (pure ivermectin) of the non-covalent IVM complex, while patient #2 remained untreated. Blood oxygen saturation levels were monitored at specific time intervals after administration (0, 24, 48, 72, 96 hours). Table 4 illustrates the changes in oxygen saturation levels for each patient from Day 0 to Day 4. As depicted in Table 11, the patient who received the non-covalent IVM complex demonstrated a rapid recovery in oxygen saturation levels, which are associated with SARS-CoV-2 infection, in contrast to the untreated patient. These data indicate that the non-covalent complex of IVM effectively reduces SARS-CoV-2 replication in the lungs.

TABLE 4
Blood oxygen saturation levels without treatment and after
oral administration of non-covalent complex of IVM.

| | | Patient #1, SpO₂, % |
|---|---|---|
| | | after oral administration of |
| | | non-covalent complex of |
| | Patient #2, SpO₂, %, | IVM , 600 μg/kg (per pure |
| Time, h | without treatment | ivermectin) |
| 0 h | 94 | 89 |
| 24 h | 93 | 94 |
| 48 h | 93 | 95 |
| 72 h | 92 | 95 |
| 96 h | 92 | 96 |

### COVID-19 Patient Study M2: Viral Load

The viral load of a symptomatic male patient who tested positive for SARS-CoV-2 was measured. The patient's condition was moderately severe, with a rapid decrease in oxygen saturation levels. Patient had a BMI of 36.9, indicating a Class 2 obesity, as well as prediabetes and stage 2 hypertension. These conditions are characterized by a high presence of ACE2 receptors, to which the SARS-CoV-2 virus binds, enabling its entry into cells. The SARS-CoV-2 viral load from nasopharyngeal swabs was quantified from samples stored at −40° C. until use. Viral RNA was isolated using the QIAamp Viral RNA Mini Kit (Qiagen, Hilden, Germany) from stored samples. Quantitative reverse transcriptase PCR (RT-qPCR) targeting the SARS-CoV-2 N-gene was then performed. The standard curve consisted of in vitro transcribed viral RNA serially diluted in a matrix of cellular RNA from nasopharyngeal negative samples.

This assay included measurement of the housekeeping gene as an internal control and normalizer. The cycle threshold (Ct) of the housekeeping gene was used to correct the specific SARS-CoV-2 Ct according to the number of cells in the sample. Therefore, viral load measurements were expressed in log₁₀ copies per reaction. The log₁₀ viral load before oral administration of non-covalent complex of IVM was 6.3 copies per mL, and log₁₀ viral load 24 hours after administration 1200 μg/kg (per pure IVM) of non-covalent complex of IVM was 3.1 copies per mL. The log₁₀ viral load was significantly different between the start of treatment, and 24 hours after the start of treatment. These data suggest that non-covalent complex of IVM effectively reduces replication of SARS-CoV-2.

TABLE 5
Viral load levels before and 24 h after oral administration
of non-covalent complex of IVM, 1200 μg/kg.

| | log₁₀ copies per mL, after oral administration of non-covalent |
|---|---|
| Time, h | complex of IVM, 1200 μg/kg (per pure ivermectin) |
| 0 h | 6.3 |
| 24 h | 3.1 |

After consuming the complex, the symptoms of the illness had vanished within twelve hours, leaving only a lingering sense of general weakness, which gradually dissipated over the course of several days. Oxygen saturation levels returned to normal within two days, and no post-COVID (long COVID) symptoms were observed.

### COVID-19 Patient Study No3: Viral Load

The viral load of a symptomatic female patient (absence of chronic diseases) who tested positive for SARS-CoV-2 was measured. The log₁₀ viral load before oral administration of non-covalent complex of IVM was 7.2 copies per mL, and log₁₀ viral load 24, 48, 72 hours after administration 600 μg/kg (per pure ivermectin) of non-covalent complex of IVM was 3.9, 1.5, 0 copies per mL. The log₁₀ viral load was significantly different between the start of treatment and 24, 48, 72 hours after the start of treatment. These data suggest that non-covalent complex of IVM effectively reduces replication of SARS-CoV-2.

TABLE 6
Viral load levels before and 24 h after oral administration
of non-covalent complex of IVM, 600 μg/kg.

| | log₁₀ copies per mL, after oral administration of non-covalent |
|---|---|
| Time, h | complex of IVM, 600 μg/kg (per pure ivermectin) |
| 0 h | 7.2 |
| 24 h | 3.9 |
| 48 h | 1.5 |
| 72 h | 0 |

After consuming the complex, the symptoms of the illness had vanished within twelve hours.

### Dengue Patient Study No1: Temperature

A female patient tested positive for the NS1 test, which detects the nonstructural NS1 protein of the dengue virus. She presented with symptoms, including a fever of 39.5° C./103° F., severe headaches, pain behind the eyes, nausea, vomiting, a skin rash, as well as muscle and joint pains. The patient was given a non-covalent complex of IVM mixed with 300 ml of water at a dosage of 700 μg/kg (equivalent to pure ivermectin). The compound was administered orally. The patient received the medication one day after her fever had reached 39.5° C./103° F. and a day and a half after the fever had initially started. Body temperature was periodically monitored immediately after administration (under the armpit). The patient's fever gradually subsided, muscle pain improved, and within 10 hours, the disease symptoms disappeared. The patient returned to her regular activities after 10 hours.

TABLE 7
Temperature of a dengue patient after administration
of non-covalent complex of IVM

| | |
|---|---|
| Time | T, ° C. |

| | | |
|---|---|---|
| 0 | h | 39.2 |
| 1 | h | 39.3 |
| 2 | h | 39.4 |
| 3 | h | 39.0 |
| 4 | h | 38.7 |
| 5 | h | 38.3 |
| 10 | h | 37.3 |
| 13 | h | 37.2 |
| 15 | h | 37.2 |
| 17 | h | 37.4 |
| 19 | h | 37.4 |
| 21 | h | 37.4 |
| 23 | h | 37.5 |
| 25 | h | 37.8 |
| 27 | h | 37.6 |
| 29 | h | 37.3 |
| 37 | h | 37.2 |
| 39 | h | 37.3 |
| 41 | h | 37.3 |
| 43 | h | 37.2 |
| 45 | h | 37.0 |
| 47 | h | 37.2 |
| 49 | h | 37.3 |
| 51 | h | 37.3 |
| 61 | h | 36.7 |
| 62 | h | 36.9 |
| 64 | h | 36.9 |
| 67 | h | 37.0 |
| 69 | h | 37.0 |
| 71 | h | 37.1 |

### Dengue Patient Study No2: Temperature

A male patient tested positive for NS1 test, which detects the nonstructural NS1 protein of the dengue virus. Symptoms include fever of 40° C./104° F., severe headache, nausea, muscle aches, lymphadenopathy. The patient received the non-covalent complex of IVM as a mixture with 300 ml water of 800 μg/kg (per pure ivermectin). The compound is administered orally. The patient's fever gradually subsided, fever's down to 37.5° C./99.5° F. in 12 hours, muscle pain improved, and within 12 hours, the disease symptoms disappeared. Lymphadenopathy was gone within 2 weeks without treatment.

### IX. CONCLUDING REMARKS

Names of compounds of the present disclosure are provided using ACD/Name software for naming chemical compounds (Advanced Chemistry Development, Inc., Toronto, Canada), or IUPAC or IUPAC condensed naming convention, or using Symbol Nomenclature For Glycans (SNFG) or Oxford Notation. Other compounds or radicals may be named with common names or systematic or non-systematic names.

When trade names are used herein, applicants intend to independently include the trade name product and the active pharmaceutical ingredient(s) of the trade name product.

The compounds of the present invention can be prepared by methods known to one of skill in the art.

The invention has been described with reference to various specific and preferred embodiments and techniques. However, one skilled in the art will understand that many variations and modifications may be made while remaining within the spirit and scope of the invention.

While the present invention has been described in detail and with reference to specific embodiments thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents. The reference to weight percents includes the weight percent of the component in the completed Formula, unless noted otherwise.

Although the disclosed inventions are illustrated and described herein as embodied in one or more specific examples, it is nevertheless not intended to be limited to the details shown, since various modifications and structural changes may be made therein without departing from the scope of the inventions. In addition, various features from one of the embodiments may be incorporated into another of the embodiments. Accordingly, it is appropriate that the invention be construed broadly and in a manner consistent with the scope of the disclosure.

Having described preferred embodiments, it is believed that other modifications, variations and changes will be suggested to those skilled in the art in view of the teachings set forth herein. It is therefore to be understood that all such variations, modifications and changes are believed to fall within the scope of the present invention as defined by the appended claims. Although specific terms are employed herein, they are used in a generic and descriptive sense only and not for purposes of limitation.

What is claimed is:

1. Non-covalent active pharmaceutical ingredient-carrier complex (of Formula I)
[GUEST]k·[HOST]n·(H2O)m
with enhanced permeability and/or bioavailability properties for treating cancer in a human as well as for manufacturing a pharmaceutical dosage form to treat cancer in a human in need thereof (by administering a therapeutically effective amount), at a dosage ranging from 0.0001 ng/kg to 100000 mg/kg (calculated for pure API)
wherein k is 1 to 100; wherein n is 1 to 10000; wherein m is 1 to 100000;
wherein non-covalent complex is obtained by:
1) dissolution in water of solid-phase non-covalent complex or inclusion complex or molecular dispersion obtained by mechanochemical method or any method selected from:
a) grinding/milling with high energy stress method;
b) grinding/milling with high energy stress and solvent method;
c) media milling method;
d) kneading method;
e) hot-melt method/melting method/fusion method;
f) hot-melt extrusion/hot-stage extrusion method;
g) meltrex method;
h) melt agglomeration method;
i) high-pressure homogenization method;
j) solvent evaporation method;
k) spin-coated films method; electrostatic spinning, electrostatic blowing, electrospraying film casting;
l) spray-drying method;
m) supercritical fluid (SCF) process method;
n) cryogenic techniques method;
o) lyophilization/freeze-drying technique method;
p) spray freezing onto cryogenic fluids method;
q) spray freezing into cryogenic liquids (SFL) method;
r) spray freezing into vapor over liquid (SFV/L) method;
s) ultra-rapid freezing method;
t) precipitation/co-precipitation method;
u) microwave irradiation method;
v) energy input method;
w) heat/shear energy input method;
x) gel entrapment technique;
y) a method comprising: contacting an active pharmaceutical ingredient and a matrix “host substance” to form a solid dispersion under conditions sufficient to form a solid dispersion;
y) combined method.
2) any other method described in description of the patent application;
wherein the non-covalent complex has enhanced permeability (into the internal organs and tissues of the person receiving the drug) and/or bioavailability properties;
wherein the therapeutic efficacy of the non-covalent complex can be (or is) enhanced by enhanced permeability and/or bioavailability, which allows for rapid achievement of the required concentration in the organs and tissues of the patient;
wherein enhanced permeability and/or bioavailability results in at least one of the following:
a. an increase in the permeability of the API, API-carrier system (solution, non-covalent complex, and the like);
b. an increase in the concentration of API, API-carrier system in cells and tissues;
for at least one of the following reasons:
i. inhibition or modulation of multidrug resistance (MDR), predominantly mediated by efflux transporters of the ATP-binding cassette (ABC) superfamily including P-glycoprotein (P-gp);
ii. induction, inhibition or modulation of CYP1, CYP2, CYP3 enzymes (non limiting examples:
CYP1A1, 1A2, 2A6, 2B6, 2C8, 2C9, 2C19, 2D6, 2E1 or 3A4 enzymes);
iii. due to a change in the structure of the cell membrane, change in lipid biolayer properties after the incorporation of carrier substance molecules (or carrier molecular part of NCC or conjugate) into this membrane or due adhesion to this membrane;
iv. due to changes in charge, hydrophilicity, hydrophobicity or other biophysical properties of the non-covalent complex due to which changes in the transmembrane (passive, active) or paracellular transport of the non-covalent complex;
v. due to other reasons e.g., by the introduction or presence in the carrier of targeted fragments capable of selectively interacting with the receptor, cell surface receptors, membrane receptors, transmembrane receptors. And when low molecular weight compounds, peptides, aptomers, and the like are used as vector molecules and are described in the patent or scientific literature; or by other reasons described in the patent or scientific literature;
wherein GUEST is
a) 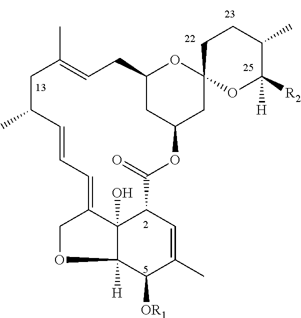
wherein
1) R₁ is H R₂ is CH₃ (i.e. GUEST is Milbemycin A3);
2) R₁ is H; R₂ is CH₂CH₃; (i.e. GUEST is Milbemycin A4);
3) R₁ is H; R₂ is CH(CH₃)₂ (i.e. GUEST is Milbemycin D);
4) R₁ is CH₃; R₂ is CH₃ (i.e. GUEST is Milbemycin B2);
5) R₁ is CH₃; R₂ is CH₂CH₃ (i.e. GUEST is Milbemycin B3);
6) R₁ is CH₃; R₂ is CH(CH₃)₂ (i.e. GUEST is Milbemycin G);
b) 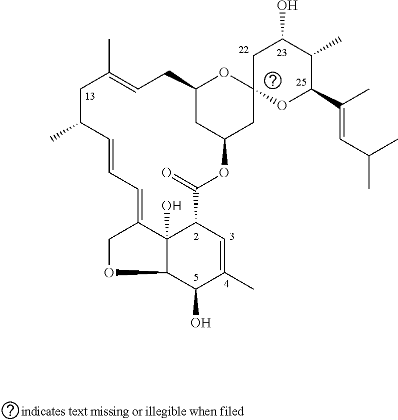 [SMILES: CC1=C[C@H]2C(=O)O[C@H]3C[C@@H](C/C=C(\C)C[C@@H](C)/C=C/C=C4\CO[C@H]([C@@H]1O)[C@@]42O)O[C@]1(C3)C[C@H](O)[C@H](C)[C@@H](/C(C)=C/C(C)C)O1]
(i.e. GUEST is Nemadectin);
c) 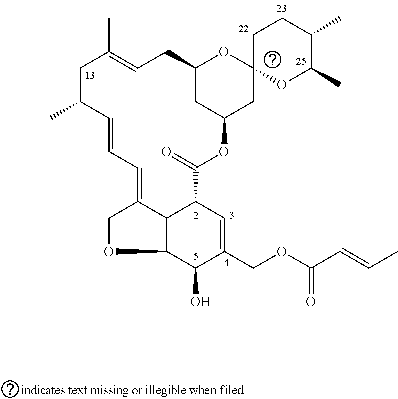
(i.e. GUEST is Meilingmycin A);
d) 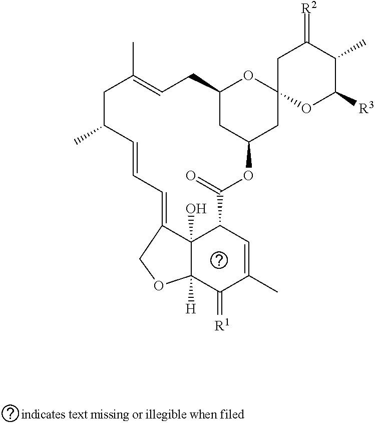
wherein
1) ═R¹ is —H(β)—OH; ═R₂ is —H, —H; —R³ is selected from —CH₃, —CH₂CH₃; (i.e. GUEST is Milbemectin);
2) ═R¹ is ═NOH; ═R₂ is —H, —H; —R³ is selected from —CH₃, —CH₂CH₃; (i.e. GUEST is Milbemycin oxime);
3) ═R¹ is —H, (β)—OH; ═R₂ is ═NOCH₃; —R³ is (Z)—C(CH₃)═CH—CH(CH₃)₂; (i.e. GUEST is Moxidectin);
4) ═R¹ is —H, (β)—OH; ═R₂ is —H, (α)—OH; —R³ is (Z)—C(CH₃)═CH—CH(CH₃)₂; (i.e. GUEST is Nemadectin);
e) 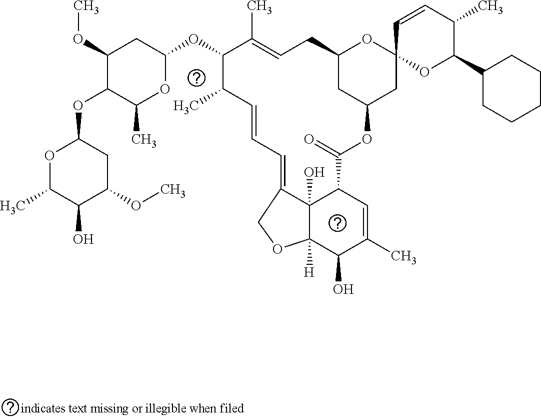 [SMILES: CO[C@H]1C[C@H](O[C@@H]2/C(C)=C/C[C@@H]3C[C@@H](C[C@]4(C=C[C@H](C)[C@@H](C5CCCCC5)O4)O3)OC(=O)[C@@H]3C=C(C)[C@@H](O)[C@H]4OC/C(=C\C=C\[C@@H]2C)[C@]43O)O[C@@H](C)C1O[C@H]1C[C@H](OC)[C@@H](O)[C@H](C)O1]
(i.e. GUEST is Doramectin);
f) 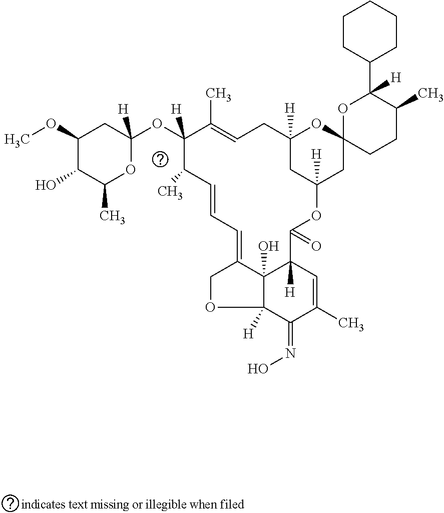
(i.e. GUEST is Selamectin);
g) 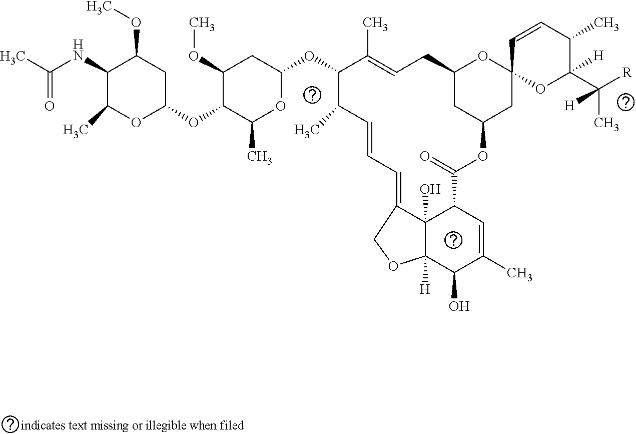
wherein
1) R is CH2CH3 (i.e. GUEST is Eprinomectin B1a);
2) R is CH3; (i.e. GUEST is Eprinomectin B1b);
h) 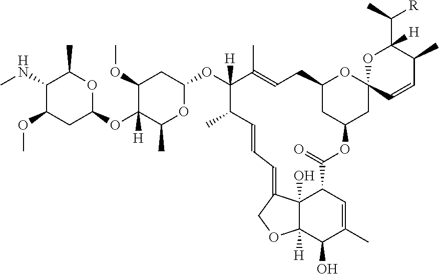
wherein
1) R is CH2CH3; (i.e. GUEST is Emamectin B1a);
2) R is CH3; (i.e. GUEST is Emamectin B1b);
i) 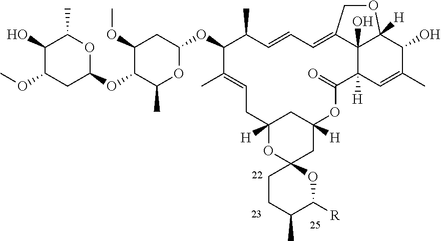
wherein
1) R is CH2CH3; (i.e. GUEST is Tenvermectin B);
2) R is CH3; (i.e. GUEST is Tenvermectin A);
j) 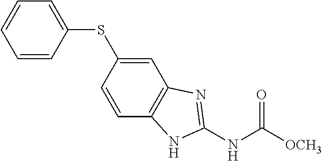 [SMILES: COC(=O)Nc1nc2cc(Sc3ccccc3)ccc2[nH]1]
(i.e. GUEST is Fenbendazole);
k) 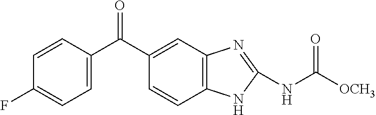 [SMILES: COC(=O)Nc1nc2cc(C(=O)c3ccc(F)cc3)ccc2[nH]1]
(i.e. GUEST is Flubendazole);
l) 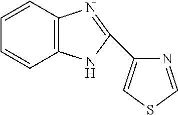 [SMILES: c1ccc2[nH]c(-c3cscn3)nc2c1]
(i.e. GUEST is Thiabendazole (Tiabendazole));
m) 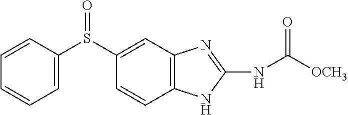 [SMILES: COC(=O)Nc1nc2cc(S(=O)c3ccccc3)ccc2[nH]1]
(i.e. GUEST is Oxfendazole);
n) 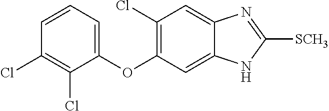 [SMILES: CSc1nc2cc(Cl)c(Oc3cccc(Cl)c3Cl)cc2[nH]1]
(i.e. GUEST is Triclabendazole);
o) 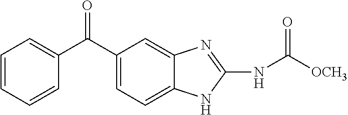 [SMILES: COC(=O)Nc1nc2cc(C(=O)c3ccccc3)ccc2[nH]1]
(i.e. GUEST is Mebendazole);
p) 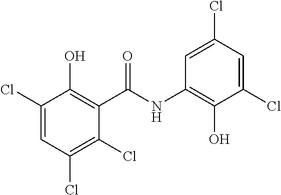 [SMILES: O=C(Nc1cc(Cl)cc(Cl)c1O)c1c(O)c(Cl)cc(Cl)c1Cl]
(i.e. GUEST is Oxyclozanide);
q) 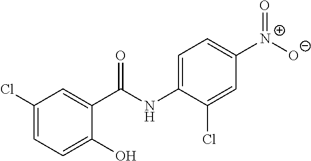 [SMILES: O=C(Nc1ccc([N+](=O)[O-])cc1Cl)c1cc(Cl)ccc1O]
(i.e. GUEST is Niclosamide);
r) 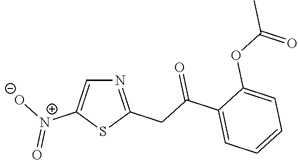 [SMILES: CC(=O)Oc1ccccc1C(=O)Cc1ncc([N+](=O)[O-])s1]
(i.e. GUEST is Nitazoxanide);
s) 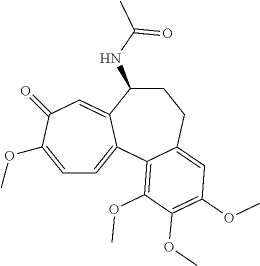
(i.e. GUEST is Colchicine);
t) 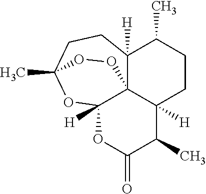
(i.e. GUEST is Artemisinin);
u) 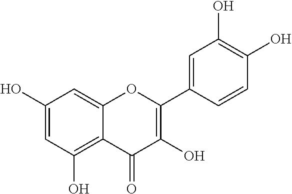 [SMILES: O=c1c(O)c(-c2ccc(O)c(O)c2)oc2cc(O)cc(O)c12]
(i.e. GUEST is Quercetin);
v) 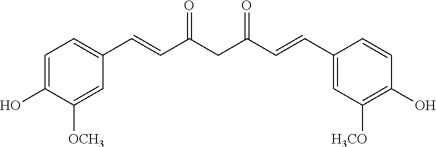 [SMILES: COc1cc(/C=C/C(=O)CC(=O)/C=C/c2ccc(O)c(OC)c2)ccc1O]
(i.e. GUEST is Curcumin);
w) 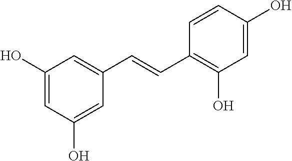 [SMILES: Oc1cc(O)cc(/C=C/c2ccc(O)cc2O)c1]
(i.e. GUEST is Resveratrol);
x) 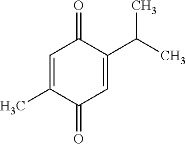
(i.e. GUEST is thymoquinone);
y) 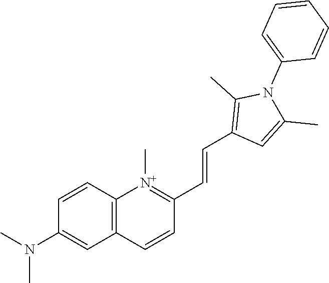 [SMILES: Cc1cc(/C=C/c2ccc3cc(N(C)C)ccc3[n+]2C)c(C)n1-c1ccccc1]
(i.e. GUEST is Pyrvinium);
z) 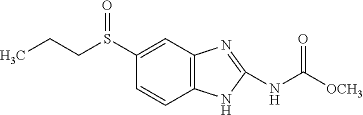 [SMILES: CCCS(=O)c1ccc2[nH]c(NC(=O)OC)nc2c1]
(i.e. GUEST is Albendazole sulfoxide (S-oxide));
aa) 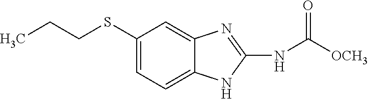 [SMILES: CCCSc1ccc2[nH]c(NC(=O)OC)nc2c1]
(i.e. GUEST is Albendazole);
ab) GUESTS selected from those listed in from FIG. 12 to FIG. 15;
ac) derivatives of compounds specified in (a)-(ab) such as racemate, enantiomer, diastereomer, tautomer, polymorph, pseudopolymorph, hydride or solvate thereof; or a pharmaceutically acceptable salt or ester or ether thereof; or prodrug thereof; or methabolite thereof; or aglycone thereof; or derivative thereof (see “DEFINITIONS OF THE PRESENT INVENTION” [0036]); or derivatives which may be obtained by gene replacement processes in genetically engineered strains, or/and by fermentation, or/and by chemical modifications and the like, or/and which are described in the scientific and patent literature;
wherein [HOST]n is
A) selected from: 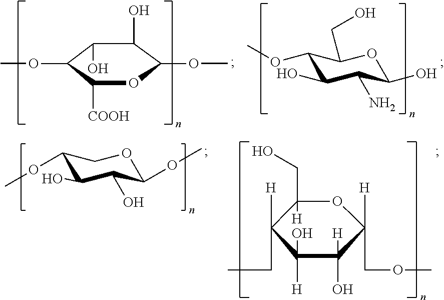
R is H, Me 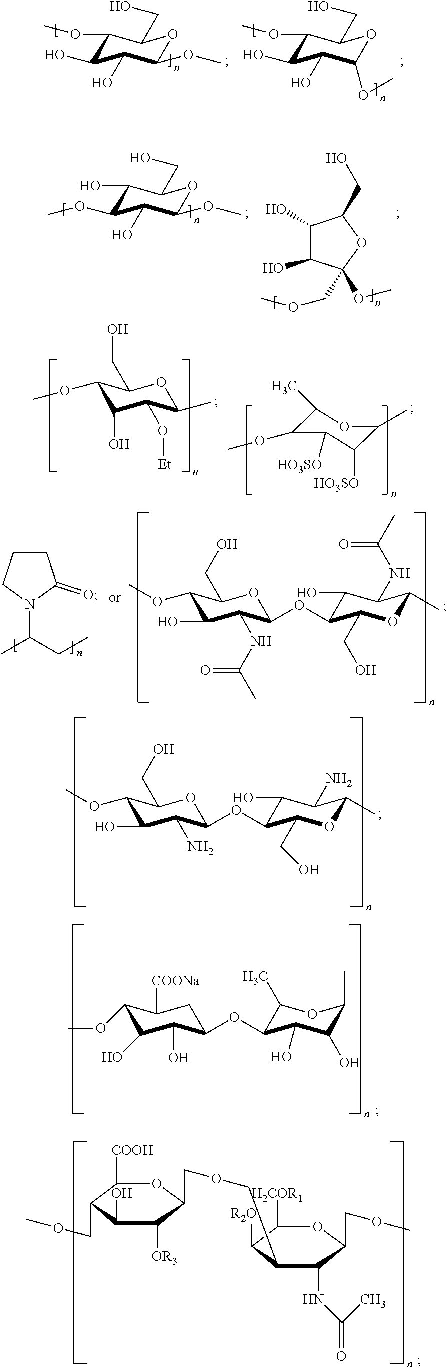 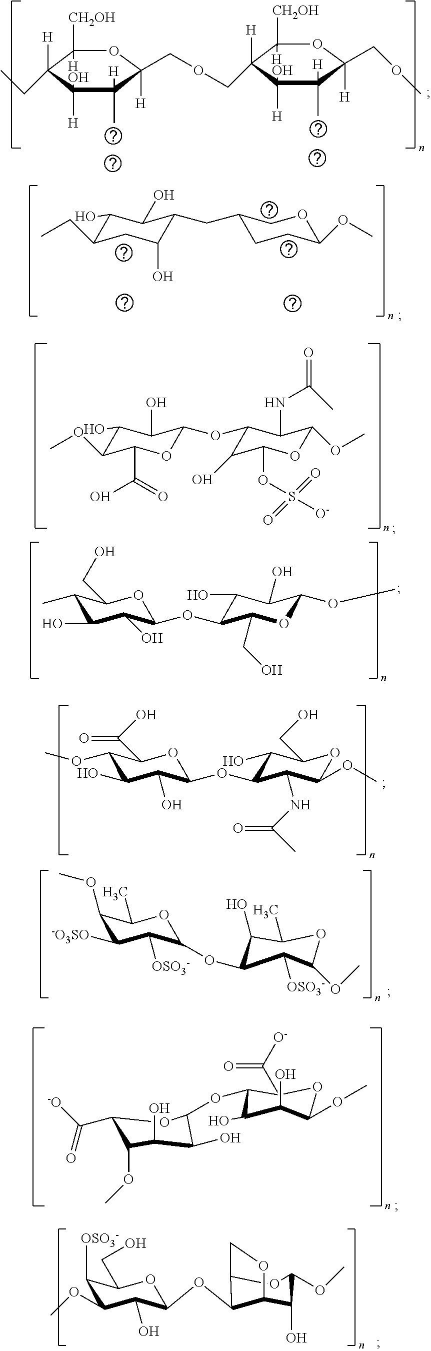 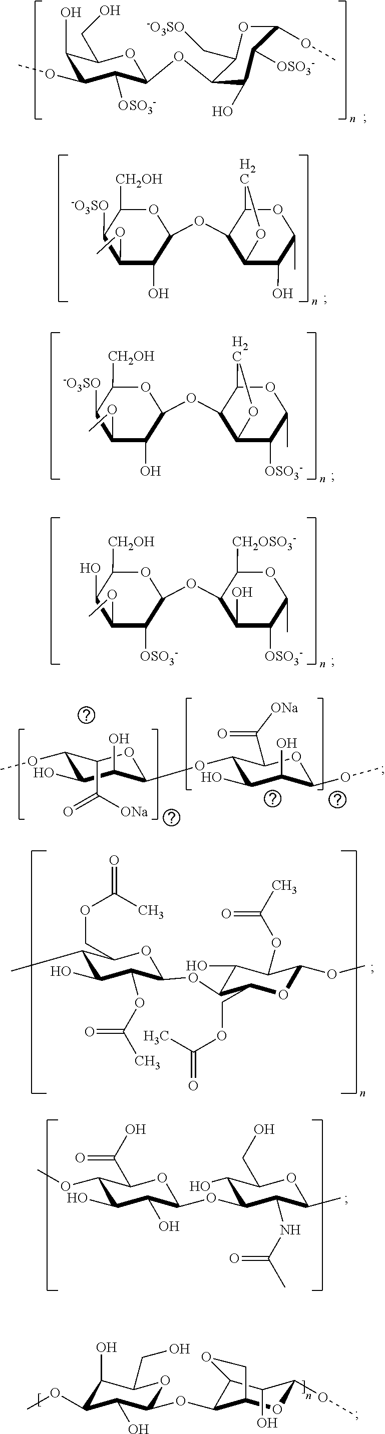 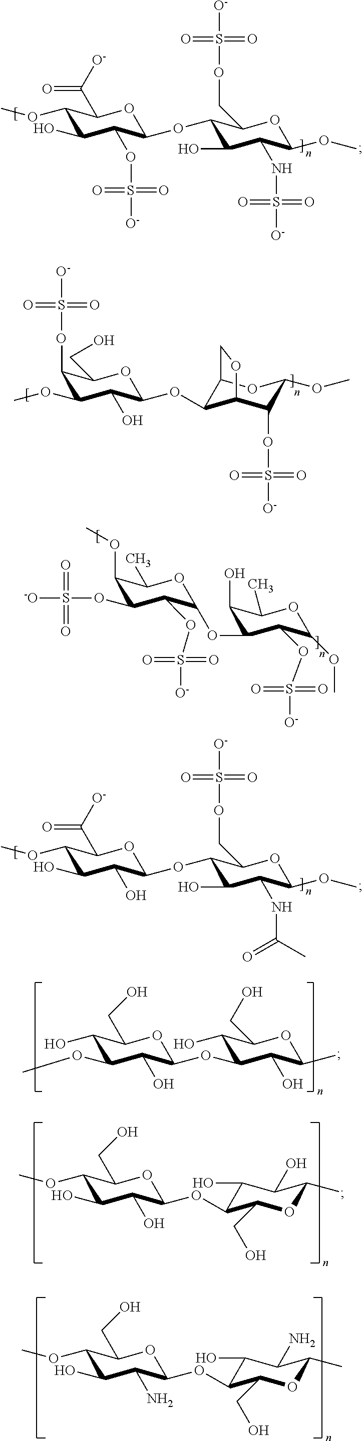 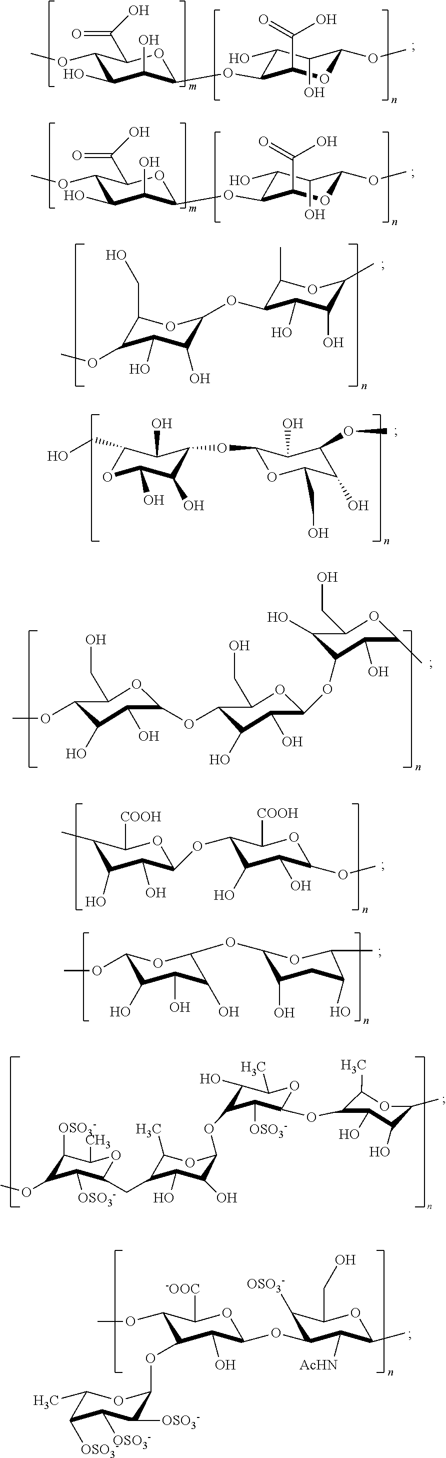 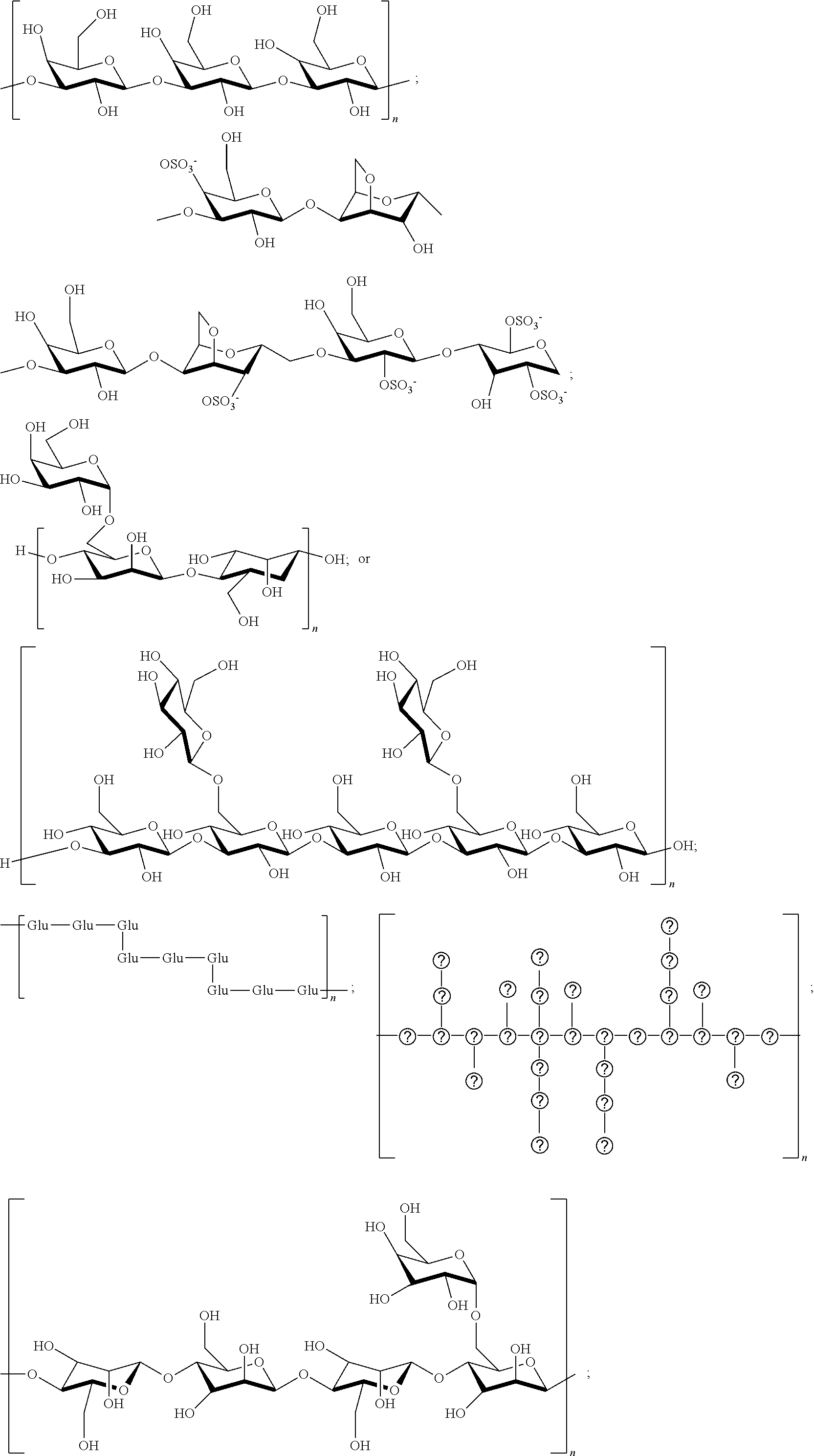 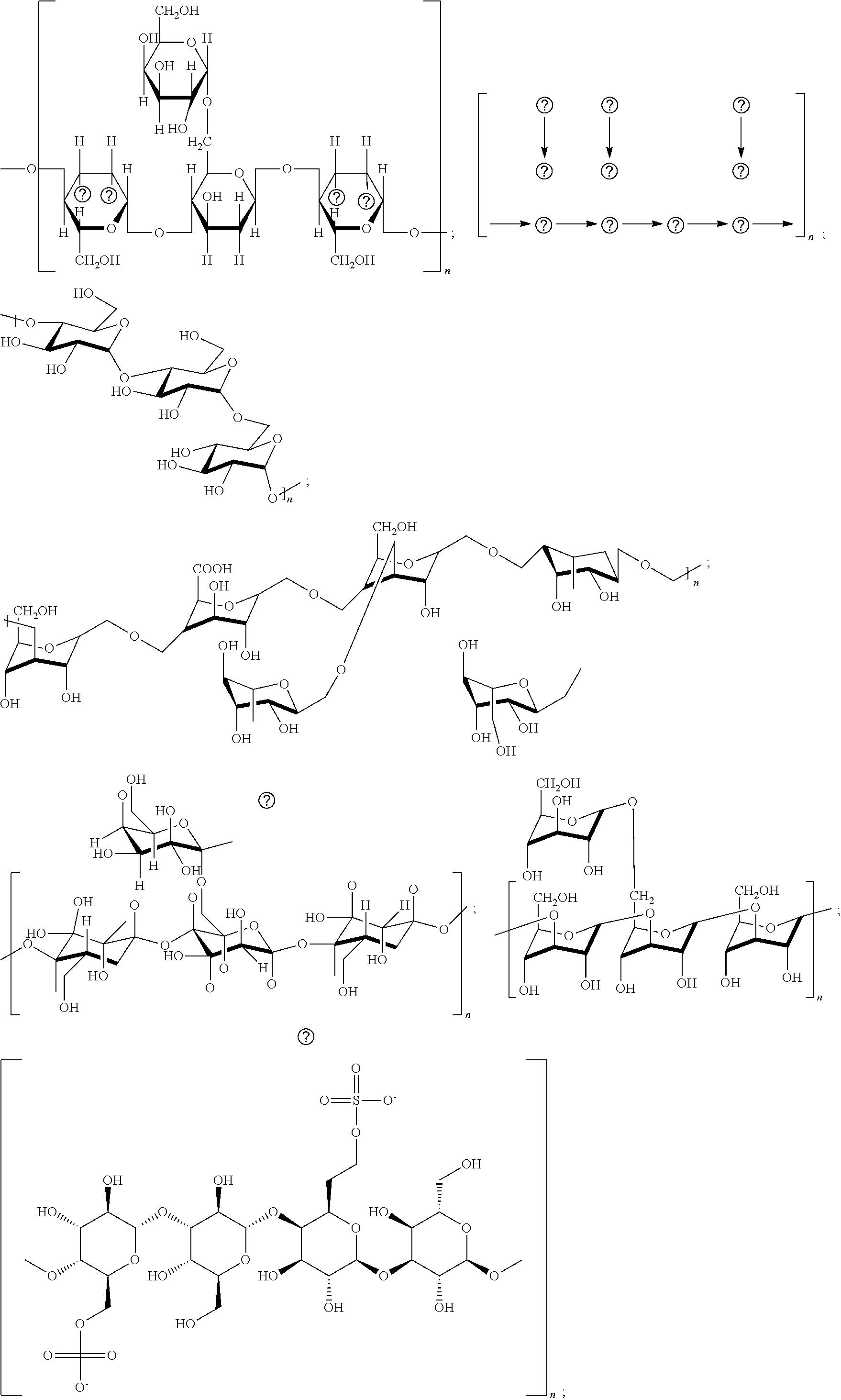 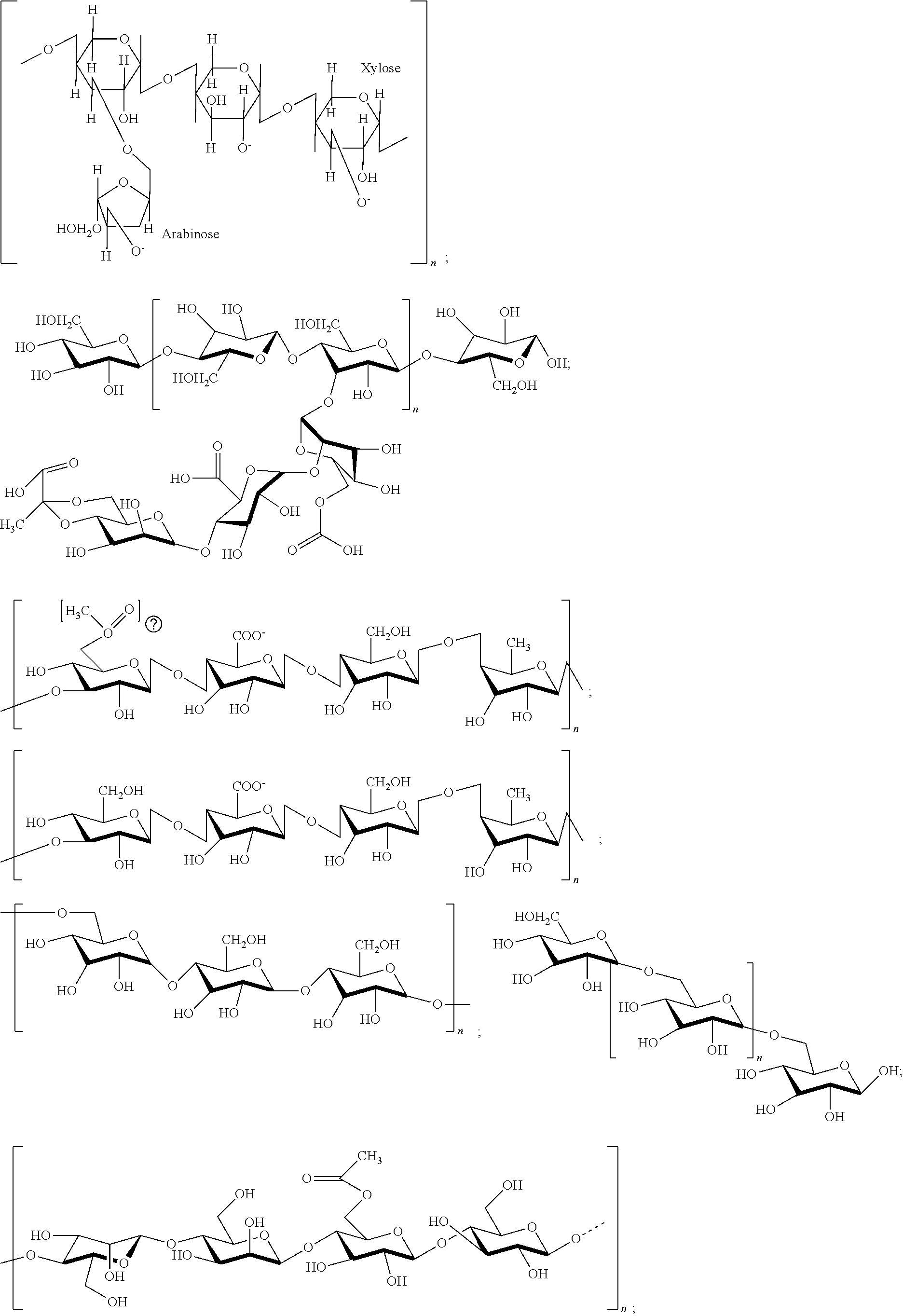 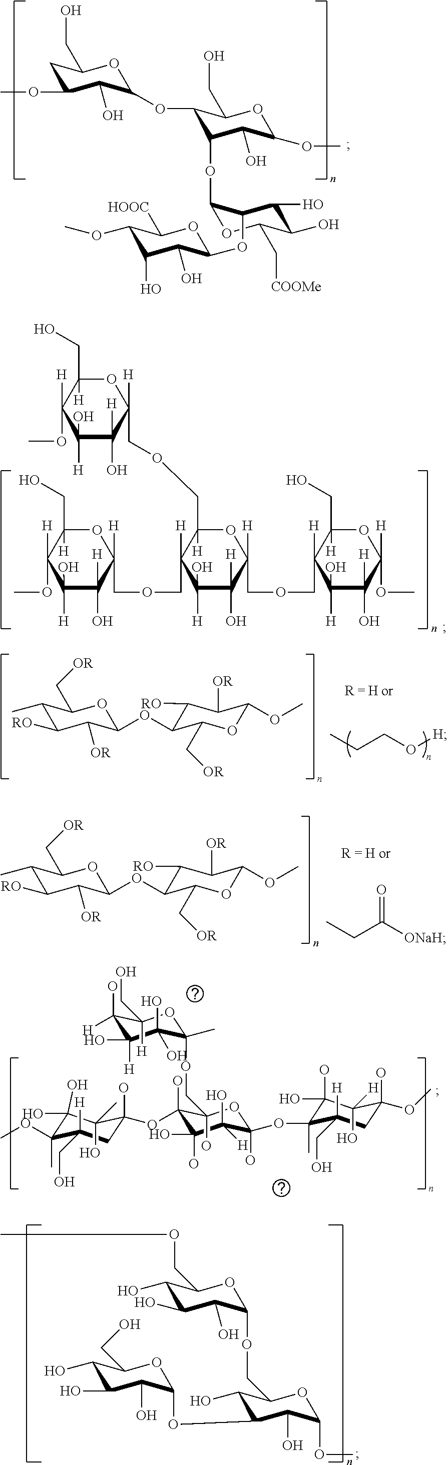 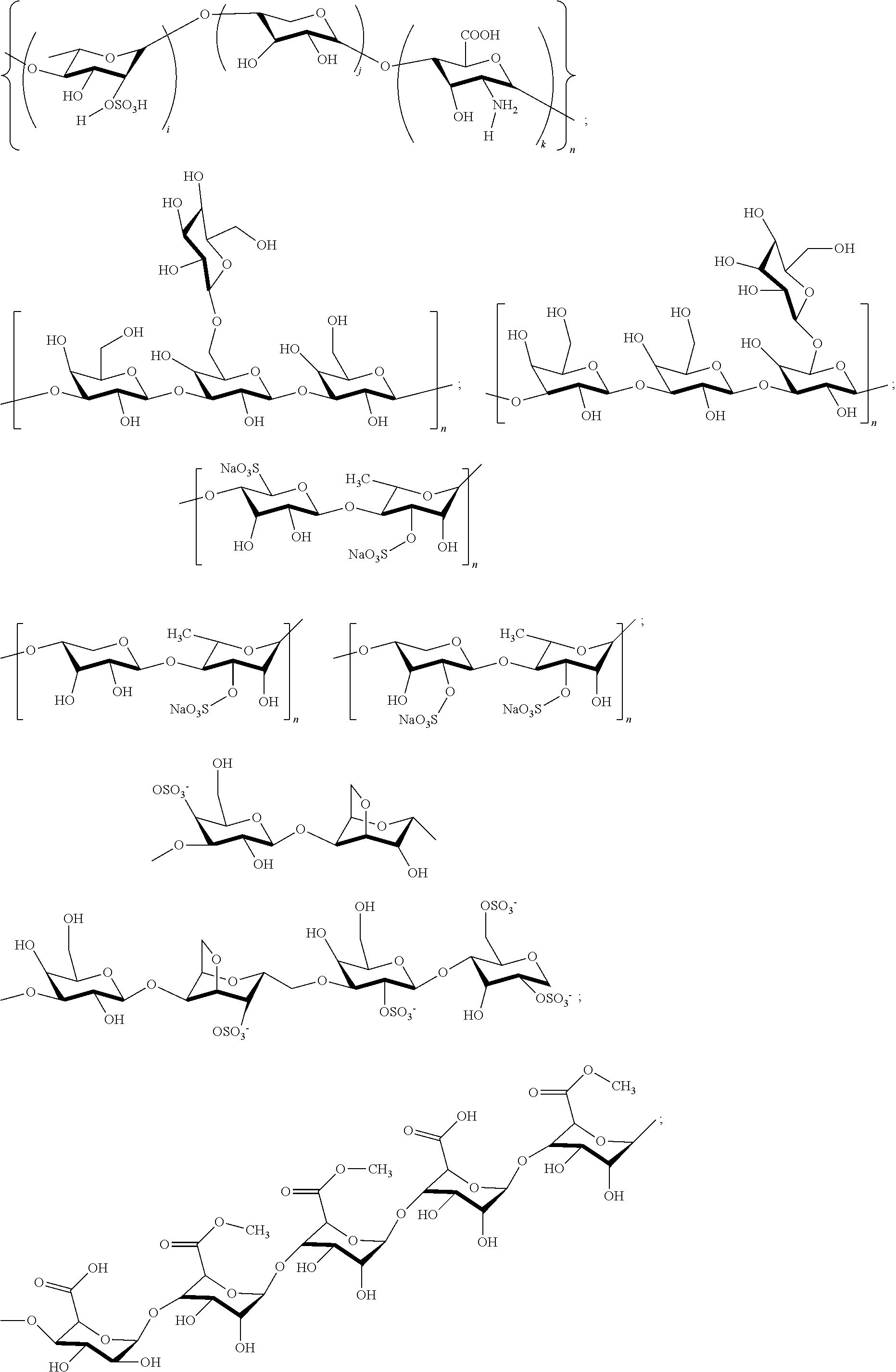 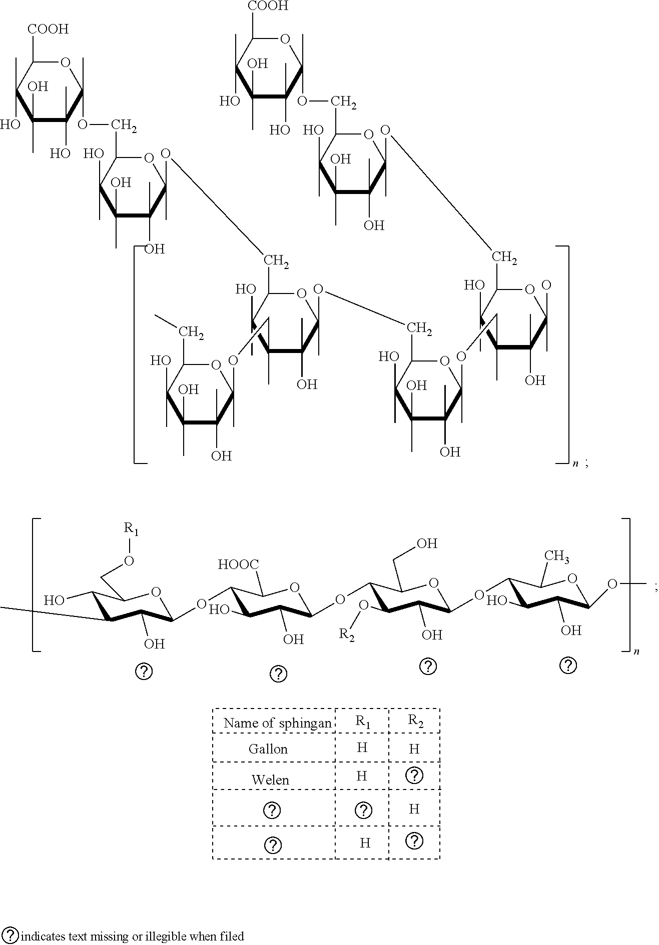 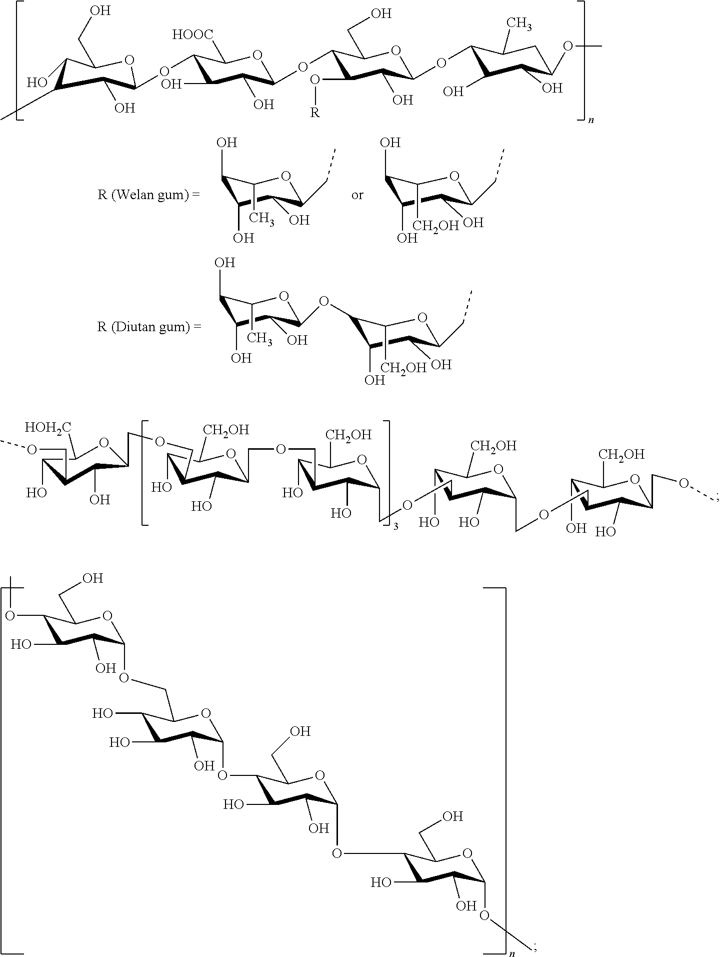 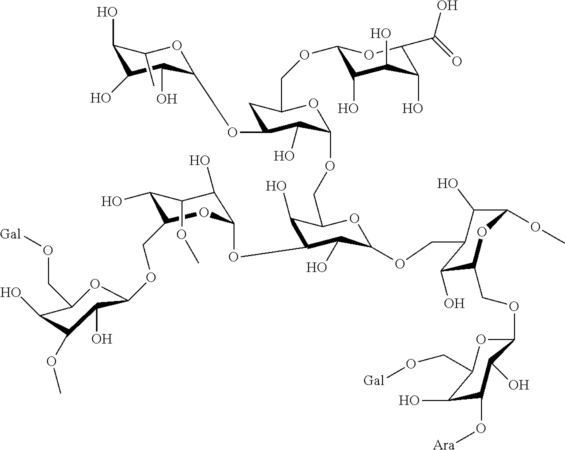
B) Chemically modified derivatives of (A) where any —OH, H can be replaced by R,
wherein R is H, halogen, F, Cl, Br, I, OH, Me, Et, Ac, OMe, OEt, OAc, OR^{A}, N(R^{A})₂, N₃, CN, NO₂, S(O)_{z}R^{A}, (C₁-C₁₅)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, —C(═O)R^{A}, —C(═O)OR^{A}, —C(═O)NR^{A}R^{B}, —C(═O)SR^{A}, —S(O) R^{A}, —S(O)₂R^{A}, —S(O)(OR^{A}), —S(O)₂(OR^{A}), —SO₂NR^{A}R^{B}, (C₆-C₂₀)aryl (C₁-C₈)alkyl, —PO(OH)₂, —PO(OH)OPO(OH)₂, —PO(OH)OPO(OH)O PO(OH)₂, —PO(OR^{A})₂, —POR^{A}R^{B}, phenyl, 1-naphthyl, 2-naphthyl, benzyl, (C₃-C₆) cycloalkyl, —CH₂—(C₃-C₆) cycloalkyl, —O—(C₁-C₈)alkyl, —O-benzyl, —O—CH₂—(C₃-C₆) cycloalkyl, CF₃, C(halogen)₃, —C(=Q)R^{A}, —C(=Q)OR^{A}, —C(=Q)N(R^{A}), —NR^{A}R^{B}, —⁺N(R^{A})₃, —SR^{A}, —S(O)R^{A}, —S(O)₂R^{A}, —S(O) (OR^{A}), —S(O)₂(OR^{A}), —OC(=Q)R^{A}, —OC(=Q) OR^{A}, —OC(=Q) (N(R^{A})₂), —SC(=Q)R^{A}, —SC(=Q)OR^{A}, —SC(=Q)(N(R^{A})₂), —N(R^{A})C(=Q)R^{A}, —N(R^{A})C(=Q)OR^{A}, —N(R^{A})C(=Q)N(R^{A})₂, —SONR^{A}₂, —CN, —N₃, —NO₂, —OR^{A}, NR^{A}R^{B}, N(R^{A})OR^{B}, NR^{A}NR^{B}, N₃, NO, NO₂, CHO, CN, —CH(=NR^{A}), —CH═NNHR^{A}, —CH═N (OR^{A}), —CH(OR^{A})₂, —C(═O)NR^{B}R^{A}, —C(═S)NR^{B}R^{A}, —C(═O)OR^{A}, (C₆-C₂₀) optionally substituted aryl, optionally substituted heteroaryl, —C(═O) (C₁-C₈)alkyl, —S(O)ₙ(C₁-C₈)alkyl, SR^{A}, or selected from radicals obtained by removal of a hydrogen atom from the compounds shown in FIG. 8
wherein Q is O, S, NR^{A}, ⁺N(O)(R^{A}), N(OR^{A}), ⁺N(O)(OR^{A}), or N—NR^{A}₂;
wherein R^{A}, R^{B} is H, halogen, F, Cl, Br, I, OH, Et, Me, Ac, OR^{C}, N(R^{C})₂, N₃, CN, NO₂, S (O)_{z}R^{C}, (C₁-C₁₅) alkyl, (C₄-C₈) carbocyclylalkyl, (C₁-C₈) substituted alkyl, (C₂-C₈)alkenyl, (C₂-C₈)substituted alkenyl, (C₂-C₈)alkynyl, (C₂-C₈)substituted alkynyl, —C(═O)R^{C}, —C(═O)OR^{C}, —C(═O)NR^{C}R^{C}, —C(═O)SR^{C}, —S(O)R^{C}, —S(O)₂R^{C}, —S(O)(OR^{C}), —S(O)₂(OR^{C}), SO₂NR^{C}R^{C}, (C₆-C₂₀)aryl(C₁-C₈)alkyl, —PO(OH)₂, —PO(OH)OPO(OH)₂, —PO(OH)OPO(OH)OPO(OH)₂, —PO(OR^{C})₂, —POR^{C}R^{C}, phenyl, 1-naphthyl, 2-naphthyl, benzyl, (C₃-C₆)cycloalkyl, —CH₂—(C₃-C₆)cycloalkyl, —O—(C₁-C₈) alkyl, —O-benzyl, —O—CH₂—(C₃-C₆)cycloalkyl, CF₃, C(halogen)₃, —C(O) R^{C}, —C(O)OR^{C}, —C(O)N(R^{C}), —NR^{C}R^{C}, —⁺N(R^{C})₃, —SR^{C}, —S(O) R^{C}, —S(O)₂R^{C}, —S(O)(OR^{C}), —S(O)₂(OR^{C}), —OC(O) R^{C}, —OC(O)OR^{C}, —OC(O)(N(R^{C})₂), —SC(=Q)R^{C}, —SC(=Q)OR^{C}, —SC(=Q)(N(R^{C})₂), N(R^{C})C(=Q)R^{C}, —N(R^{C})C(=Q)OR^{C}, —N(R^{C})C(=Q)N(R^{C})₂, —SO₂NR^{C}₂, —CN, —N₃, —NO₂, —OR^{C}, NR^{C}R^{C}, N(R^{C})OR^{C}, NR^{C}NR^{C}, N₃, NO, NO₂, CHO, CN, —CH(═NR^{C}), —CH═NNHR^{C}, —CH═N(OR^{C}), —CH(OR^{C})₂, —C(═O)NR^{C}R^{C}, —C(═S)NR^{C}R^{C}, —C(═O)OR^{C}, (C₆-C₂₀)optionally substituted aryl, optionally substituted heteroaryl, —C(═O)(C₁-C₈)alkyl, —S(O)_{z}(C₁-C₈)alkyl, or SR^{C};
wherein R^{C} is H, OH, F, Cl, Br, I, Me, Et, Ac, OMe, OEt, or OAc, or selected from radicals obtained by removal of a hydrogen atom from the compounds shown in FIG. 8;
wherein z is from 1 to 10.
D) wherein [HOST]ₙ is
D1) -[M1]ₙ-, -[Mₓ₁-M2ₓ₂]ₙ-, -[M1ₓ₁-M2ₓ₂-M3ₓ₃]ₙ-, -[M1ₓ₁-M2ₓ₂-M3ₓ₃-M4ₓ₄]ₙ-, -[M1ₓ₁-M2ₓ₂-M3ₓ₃-M4ₓ₄-M5ₓ₅]ₙ-, -[M1ₓ₁-M2ₓ₂-M3ₓ₃-M4ₓ₄-M5ₓ₅-M6ₓ₆]ₙ-, [M1]ₙ-J [M1ₓ₁-M2ₓ₂]ₙ-∫, [M1ₓ₁-M2ₓ₂-M3ₓ₃]ₙ-∫, [M1ₓ₁-M2ₓ₂-M3ₓ₃-M4ₓ₄]ₙ-∫, [M1ₓ₁-M2ₓ₂-M3ₓ₃-M4ₓ₄-M5ₓ₅]ₙ-∫, [M1ₓ₁-M2ₓ₂-M3ₓ₃-M4ₓ₄-M5ₓ₅-M6ₓ₆]ₙ-∫, 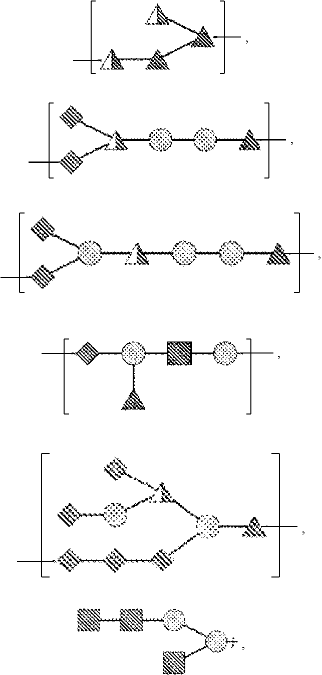
and continue on FIG. 1, FIG. 2, FIG. 3, FIG. 4, FIG. 5, and the like
wherein x1, x2, x3, x4, x5, x6 is from 1 to 10⁸;
wherein [symbol]
is M1; [symbol] is M2; [symbol] is M3; [symbol]
is M4; [symbol] is M5; [symbol] is M6;
wherein at least one of M1, M2, M3, M3, M4, M5, M6 is a repeat unit(s) or terminal residue
formed from this monomers:
a) 4-epi-Legionaminic acid; 6-Deoxy-L-altrose; N-Acetyl-6-deoxy-L-altrosamine; 6-Deoxy-D-gulose; 6-Deoxy-D-talose; N-Acetyl-6-deoxy-D-talosamine; 8-epi-Acinetaminic acid; 8-epi-Legionaminic acid; Abequose; Acinetaminic acid; D-Allose; D-Alluronic acid; D-Allosamine; N-Acetyl-D-allosamine; L-Altrose; L-Altruronic acid; L-Altrosamine; N-Acetyl-L-altrosamine; L-Apiose; L-Arabinose; Bacillosamine; Colitose; D-glycero-D-manno-Heptose; 3-Deoxy-D-lyxo-heptulosaric acid; D-Digitoxose; D-Fructose; L-Fucose; N-Acetyl-L-fucosamine; D-Galactose; D-Galacturonic acid; D-Galactosamine; N-Acetyl-D-galactosamine; D-Glucose; D-Glucuronic acid; D-Glucosamine; N-Acetyl-D-glucosamine; D-Gulose; D-Guluronic acid; D-Gulosamine; N-Acetyl-D-gulosamine; L-Idose; L-Iduronic acid; L-Idosamine; N-Acetyl-L-idosamine; 2-Keto-3-deoxy-nononic acid; 3-Deoxy-D-manno-octulosonic acid; Legionaminic acid; L-glycero-D-manno-Heptose; D-Lyxose; D-Mannose; D-Mannuronic acid; D-Mannosamine; N-Acetyl-D-mannosamine; Muramic acid; N-Acetylmuramic acid; N-Glycolylmuramic acid; Neuraminic acid; N-Acetylneuraminic acid; N-Glycolylneuraminic acid; Olivose; Paratose; Pseudaminic acid; D-Psicose; D-Quinovose; N-Acetyl-D-quinovosamine; L-Rhamnose; N-Acetyl-L-rhamnosamine; D-Ribose; Sialic acid; L-Sorbose; D-Tagatose; D-Talose; D-Taluronic acid; D-Talosamine; N-Acetyl-D-talosamine; Tyvelose; D-XyloseDioses; Aldodiose; Glycolaldehyde; Trioses; Aldotriose; Glyceraldehyde; Ketotriose; Dihydroxyacetone; Tetroses; Aldotetroses; Erythrose Threose; Ketotetrose; Erythrulose; Pentoses; Aldopentoses; Arabinose; Lyxose; Ribose; Xylose; Ketopentoses; Ribulose; Xylulose; Deoxy sugars; Deoxyribose; Hexoses; Aldohexoses; Allose; Altrose; Galactose; Glucose; Gulose; Idose; Mannose; Talose; Ketohexoses; Fructose; Psicose; Sorbose; Tagatose; Fucose; Fuculose; Rhamnose; Heptoses; Ketoheptoses; Mannoheptulose; Sedoheptulose; Octoses; Nonoses; Neuraminic acid; galactosamine, N-acetylgalactosamine, N-formylgalactosamine, N-propionylgalactosamine, N-n-butanoylgalactosamine, N-iso-butanoylgalactosamine, lactose, lactobionic acid, mannose, aminoacids, nucleosides, folate, folic acid, γ-FBA-folate, and α-FBA-folate, and other folic acid chemical derivatives;
and (using IUPAC nomenclature of organic chemistry, IUPAC Condensed code system) from this monomers:
1,4-Anhydro-Gal; 1,4-Anhydro-Gal; 1,4-Anhydro-Kdo; 1dAlt-ol; 1dEry-ol; 2,3-Anhydro-All; 2,3-Anhydro-Man; 2,3-Anhydro-Rib; 2,5-Anhydro-D-Alt; 2,5-Anhydro-D-AltOS; 2,5-Anhydro-L-Man; 2,5-Anhydro-Man; 2,5-Anhydro-Man-ol; 2,5-Anhydro-ManOS; 2,5-Anhydro-Tal-ol; 2,5-Anhydro-TalOP; 2,7-Anhydro-Kdo; 2,7-Anhydro-Kdof; 3,6-Anhydro-Fruf; 3,6-Anhydro-Gal; 3,6-Anhydro-GalOS; 3,6-Anhydro-Glc; 3,6-Anhydro-L-Gal; 3,6-Anhydro-L-GalOMe; 3dLyxHepUlosaric; 4,7-Anhydro-KdoOPEtn; 4,8-Anhydro-DDGlcOct; 4,8-Anhydro-Kdo; 4,8-Anhydro-LDGlcOct; 4dAraHex; 4dEry-ol; 4eLeg5Ac7Ac; 6dAlt; 6dAltNAc; 6dAltOAc; 6dAltf; 6dAltfOAc; 6dGul; 6dManHep; 6dTal; 6dTalNAc; 6dTalNAcOAc; 6dTalOAc; 6dTalOAcOAc; 6dTalOAcOMe; 6dTalOMe; 6dTalOMe-ol; 6dTalf; 8eAciNAcNAc; 8eLeg; 8eLeg5Ac7Ac; 8eLeg5Ac7AcGro; 8eLegNAc; 8eLegNAcNBut; Abe; AbeOAc; AcefA; AciNAcNAc; Aco; AcoNAc; AllN; AllOAc; AllOMe; Alt; AltA; AltAN; AltNAcA; AltOMeA; Altf; AltfOAc; Ami; ApiOAc; ApiOMe-ol; Apif; Ara; Ara-ol; AraGlc; AraHepUloNAc-onic; AraHepUloNAcN-onic; AraHepUloNGc-onic; AraHexA; AraN; AraNMeOMe; AraOAc; AraOAcOP-ol; AraOMe; AraOPN; Araf; ArafGro; ArafOCoum; ArafOFer; ArafOMe; ArafOS; Asc; Bac; BacNAc; BoiOMe; Col; D-2dAraHex; D-2dAraHexA; D-3dAraHepUlosonic; D-3dLyxHepUlosaric; D-3dThrHexUlosonic; D-3dThrPen; D-3dXylHexOMe; D-4dAraHex; D-4dEryHexOAcN4en; D-4dLyxHex; D-4dLyxHexOMe; D-4dThrHexA4en; D-4dThrHexAN4en; D-4dThrHexOAcN4en; D-4dXylHex; D-6dAllOMe; D-6dAlt; D-6dAltHep; D-6dAltHepOMe; D-6dAltHepf; D-6dAraHex; D-6dAraHexN; D-6dAraHexNAc; D-6dAraHexOMe; D-6dLyxHexOMe; D-6dManHep; D-6dManHepOAc; D-6dManHepOP; D-6dTal; D-6dTalHep; D-6dTalOAc; D-6dTalOAcOMe; D-6dTalOMe; D-6dXylHex; D-6dXylHexN4Ulo; D-6dXylHexNAc4Ulo; D-6dXylHexOMe; D-7dLyxOctUlosonic; D-9dThrAltNon-onic; D-Alt; D-Apif; D-ApifOAc; D-ApifOMe; D-Ara; D-Ara-ol; D-AraHepUlo-onic; D-AraHex; D-AraHexUloOMe; D-AraN; D-AraOS; D-Araf; D-ArafN; D-Fuc; D-Fuc-ol; D-FucN; D-FucNAc; D-FucNAc-ol; D-FucNAcN; D-FucNAcNMe; D-FucNAcNMeN; D-FucNAcOAc; D-FucNAcOMe; D-FucNAcOP; D-FucNAcOPEtn; D-FucNAlaAc; D-FucNAsp; D-FucNBut; D-FucNButGro; D-FucNFo; D-FucNLac; D-FucNMeN; D-FucNN; D-FucNThrAc; D-FucOAc; D-FucOAcN; D-FucOAcNBut; D-FucOAcNGroA; D-FucOAcOBut; D-FucOAcOMe; D-FucOBut; D-FucOEtn; D-FucOMe; D-FucOMeN; D-FucOMeOCoum; D-FucOMeOFer; D-FucOMeOSin; D-FucOS; D-Fucf; D-FucfNAc; D-FucfOAc; D-Ido; D-IdoA; D-IdoOSA; D-Rha; D-Rha-ol; D-RhaCMe; D-RhaGro; D-RhaN; D-RhaNAc; D-RhaNAcOAc; D-RhaNBut; D-RhaNButOMe; D-RhaNFo; D-RhaOFoN; D-RhaOMe; D-RhaOMeN; D-RhaOP; D-RhaOS; D-RibHex; D-RibHexNAc; D-Sor; D-ThrHexA4en; D-ThrHexAN4en; D-ThrHexfNAc2en; D-ThrPen; D-Thre-ol; DDA1tHep; DDAltHepOMe; DDGa1Hep; DDGa1HepOMe; DDGlcHep; DDManHep; DDManHepGroPA; DDManHepOBut; DDManHepOEtn; DDManHepOMe; DDManHepOP; DDManHepOPEtn; DDManNonUloNAcOFoN-onic; DLA1tNonUloNAc-onic; DLGalNonUloNAc-onic; DLGalNonUloNAcN; DLGalNonUloNAcN-onic; DLGlcHepOMe; DLHepGlcOMe; DLManHep; DLManHepOPEtn; Dha; Dig; DigCMe; DigOAc; DigOFo; DigOMe; Ery; Ery-L-GlcNonUloNAcOAcOMeSH-onic; Ery-ol; Ery-onic; EryHex; EryHex2en; EryHexA3en; EryOMe-onic; Fru; Fruf; FrufF; FrufI; FrufN; FrufNAc; FrufOAc; FrufOAcOBzOCoum; FrufOAcOFer; FrufOBzOCin; FrufOBzOCoum; FrufOBzOFer; FrufOFer; FrufOLau; Fuc; Fuc-ol; FucN; FucNAc; FucNAcA; FucNAcGroP; FucNAcN; FucNAcNMe; FucNAcOAc; FucNAcOMe; FucNAla; FucNAm; FucNBut; FucNFo; FucNProp; FucNThrAc; FucOAc; FucOAcNAm; FucOAcNBut; FucOAcOMe; FucOAcOSOMe; FucOMe; FucOMeOPam; FucOMeOVac; FucOP; FucOPOMe; FucOS; FucOSOMe; Fucf; Gal; Gal-ol; Gal6Ulo; GalA; GalA-ol; GalAAla; GalAAlaLys; GalAGroN; GalALys; GalAN; GalANCys; GalANCysAc; GalANSerAc; GalAOLac; GalAOPyr; GalASer; GalAThr; GalAThrAc; GalCl; GalF; GalGro; GalGroN; GalGroP; GalN; GalNAc; GalNAc-ol; GalNAc-onic; GalNAcA; GalNAcAAla; GalNAcAN; GalNAcASer; GalNAcGro; GalNAcGroP; GalNAcGroPAN; GalNAcN; GalNAcOAc; GalNAcOAcA; GalNAcOAcAN; GalNAcOAcGroP; GalNAcOAcOMeA; GalNAcOAcOP; GalNAcOMe; GalNAcOP; GalNAcOPCho; GalNAcOPEtn; GalNAcOPyr; GalNAcOS; GalNAla; GalNAmA; GalNCysGly; GalNFoA; GalNFoAN; GalNOPCho; GalNSuc; GalNonUloNAc-onic; GalOAc; GalOAcA; GalOAcAGroN; GalOAcAOLac; GalOAcAThr; GalOAcGroP; GalOAcN; GalOAcNAla; GalOAcNAmA; GalOAcNFoA; GalOAcNFoAN; GalOAcOFoA; GalOAcOMe; GalOAcOP; GalOAcOPyr; GalOFoAN; GalOFoNAN; GalOLac; GalOLac-ol; GalOMe; GalOMeA; GalOMeCl; GalOMeF; GalOMeNAla; GalOP; GalOPA; GalOPAEtn; GalOPAN; GalOPCho; GalOPEtn; GalOPEtnA; GalOPEtnN; GalOPy; GalOPyr; GalOS; GalOSA; GalOSOEt; GalOSOMeA; GalOctUloNAc-onic; Galf; GalfGro; GalfGroP; GalfNAc; GalfOAc; GalfOAcGro; GalfOAcGroP; GalfOAcOLac; GalfOLac; GalfOMe; GalfOP; GalfOPCho; GalfOPyr; Gl; Glc; Glc-ol; Glc6Ulo; GlcA; GlcAAla; GlcAAlaLys; GlcAGlu; GlcAGly; GlcAGro; GlcAGroN; GlcALys; GlcAN; GlcAOLac; GlcAOPy; GlcAOPyr; GlcASer; GlcAThr; GlcAThrAc; GlcCho; GlcF; GlcGro; GlcGroA; GlcGroP; GlcGroPA; GlcI; GlcN; GlcN-ol; GlcNAc; GlcNAc-ol; GlcNAcA; GlcNAcAAla; GlcNAcAN; GlcNAcANAla; GlcNAcANAlaAc; GlcNAcANAlaFo; GlcNAcAla; GlcNAcCl; GlcNAcGlu; GlcNAcGly; GlcNAcGro; GlcNAcGroP; GlcNAcGroPA; GlcNAcI; GlcNAcN; GlcNAcN-ol; GlcNAcNAla; GlcNAcNAlaFo; GlcNAcNAmA; GlcNAcNButA; GlcNAcOAc; GlcNAcOAcA; GlcNAcOAcN; GlcNAcOAcNAla; GlcNAcOAcOCmOOle; GlcNAcOAcOCmOPam; GlcNAcOAcOCmOVac; GlcNAcOAcOLac; GlcNAcOAcOOle; GlcNAcOAcOPam; GlcNAcOAcOPyr; GlcNAcOAcOS-ol; GlcNAcOAcOVac; GlcNAcOGc; GlcNAcOLac; GlcNAcOLacAla; GlcNAcOLacGro; GlcNAcOMe; GlcNAcOMeA; GlcNAcOP; GlcNAcOPCho; GlcNAcOPEtg; GlcNAcOPEtn; GlcNAcOPOAch; GlcNAcOPyr; GlcNAcOS; GlcNAcOS-ol; GlcNAcOSA; GlcNAm; GlcNAmA; GlcNBut; GlcNButAN; GlcNButOAc; GlcNCmOCm; GlcNCmOCmOOle; GlcNCmOCmOVac; GlcNCmOVac; GlcNGc; GlcNGly; GlcNMe; GlcNMeOCm; GlcNMeOCmOPam; GlcNMeOCmOSte; GlcNMeOCmOVac; GlcNMeOSte; GlcNMeOVac; GlcNN; GlcNOAep; GlcNOCmOAch; GlcNOCmOVac; GlcNOMar; GlcNOMe; GlcNOMyr; GlcNOOle; GlcNOPam; GlcNOPyr; GlcNOSte; GlcNOVac; GlcNS; GlcNSOS; GlcNSOSOMe; GlcNSuc; GlcOAc; GlcOAcA; GlcOAcGro; GlcOAcGroA; GlcOAcGroP; GlcOAcN; GlcOAcNBut; GlcOAcNCmOOle; GlcOAcNCmOPam; GlcOAcNCmOVac; GlcOAcNMeOCm; GlcOAcNMeOCmOVac; GlcOAcNMeOVac; GlcOAcNOCmOVac; GlcOAcNOOle; GlcOAcNOPam; GlcOAcNOVac; GlcOAcOCoum; GlcOAcOFer; GlcOAcOOle; GlcOAcOP; GlcOAcOPam; GlcOAcOS; GlcOAcOSA; GlcOAcOSte; GlcOButA; GlcOBz; GlcOCoum; GlcOEt; GlcOEtn; GlcOEtnA; GlcOEtnN; GlcOFer; GlcOFoN; GlcOGc; GlcOLac; GlcOMal; GlcOMe; GlcOMe-ol; GlcOMeA; GlcOMeAN; GlcOMeN; GlcOMeNOMyr; GlcOMeOFoA; GlcOMeOPyr; GlcOOle; GlcOP; GlcOP-ol; GlcOPA; GlcOPCho; GlcOPChoGro; GlcOPEtn; GlcOPEtnGro; GlcOPEtnN; GlcOPGroP; GlcOPN; GlcOPNOMyr; GlcOPNOPam; GlcOPOOle; GlcOPPEtn; GlcOPPEtnN; GlcOPam; GlcOPyr; GlcOS; GlcOSA; GlcOSN; GlcOSNMeOCm; GlcOSOEt; GlcOSOMe; GlcOSOMeA; GlcOSin; GlcS; GlcSH; GlcThr; Glcf; Gro; Gro-ol; Gul; GulAN; GulNAcA; GulNAcAN; GulNAcNAmA; GulNAcOAcA; Hep; HepOP; HepOPEtn; HepOPPEtn; Hex; HexA; HexN; HexNAc; HexOMeOFo; Hexf; Ido; IdoA; IdoN; IdoNAc; IdoOAcA; IdoOAcOSA; IdoOMeA; IdoOS; IdoOSA; IdoOSOEtA; IdoOSOMeA; Kdn; KdnOAc; KdnOMe; KdnOPyr; Kdo; Kdo-ol; KdoGroP; KdoN; KdoOAc; KdoOAcOS; KdoOMe; KdoOP; KdoOPEtn; KdoOPN; KdoOPOEtn; KdoOPOPEtn; KdoOPPEtn; KdoOPPEtnN; KdoOPyr; KdoOS; Kdof; Ko; KoOMe; KoOPEtn; L-4dEryHexAN4en; L-4dThrHex4en; L-4dThrHexA4en; L-4dThrHexA4enAla; L-4dThrHexAN4en; L-4dThre-ol; L-6dAraHex; L-6dAraHexOMe; L-6dGalHep; L-6dGalHepOP; L-6dGulHep; L-6dGulHepOMe; L-6dGulHepOP; L-6dXylHexNAc4Ulo; L-Aco; L-AcoOMe; L-AcoOMeOFo; L-BoiOMe; L-Cym; L-CymOAc; L-DigOMe; L-Ery; L-EryCMeOH; L-EryHexA4en; L-Fru; L-Fruf; L-Gal; L-GalAN; L-GalNAc; L-GalNAc-onic; L-GalNAcA; L-GalNAcAN; L-GalNAcOAcA; L-GalNAmA; L-GalOAcNAmA; L-GalOS; L-Glc; L-GlcA; L-GlcNAc; L-GlcOMe; L-Gro-onic; L-GroHexUlo; L-Gul; L-Gul-onic; L-GulA; L-GulAN; L-GulHep; L-GulNAc; L-GulNAcA; L-GulNAcAGly; L-GulNAcAN; L-GulNAcANEtn; L-GulNAcNAmA; L-GulNAcNEtnA; L-GulNAcOAc; L-GulNAcOAcA; L-GulNAcOAcAN; L-GulNAcOEtA; L-GulNAcOEtnA; L-GulOAcA; L-Lyx; L-LyxHex; L-LyxHexNMe; L-LyxHexOMe; L-Man; L-ManOMe; L-ManOctUlo-onic; L-Ole; L-OleOAc; L-Oli; L-OliOMe; L-Qui; L-QuiN; L-QuiNAc; L-QuiNAcOMe; L-QuiNAcOP; L-QuiOMeN; L-RibHex; L-Ribf; L-Tal; L-The; L-TheOAc; L-Thr; L-ThrHexA4en; L-ThrHexAN4en; L-ThrHexOMe4en; L-ThrHexOMeA4en; L-ThrHexOSA4en; L-Xyl; L-XylHex; L-XylOMe; LDGa1Hep; LDGalNonUloNAc-onic; LDGlcHep; LDIdoHep; LDIdoHepPro; LDManHep; LDManHepGroN; LDManHepGroPA; LDManHepOAc; LDManHepOCm; LDManHepOEtn; LDManHepOMe; LDManHepOP; LDManHepOPEtn; LDManHepOPEtnOEtn; LDManHepOPOCm; LDManHepOPOMe; LDManHepOPOPEtn; LDManHepOPOPPEtn; LDManHepOPPEtn; LDManHepOPPEtnOPyrP; LDManHepOPyrP; LDManNonUloNAcOFoN-onic; LDManNonUloOFoNN-onic; LLManNonUloOFoN-onic; Leg; Leg5Ac7Ac; LegNAc; LegNAcAla; LegNAcNAla; LegNAcNAm; LegNAcNBut; LegNFo; Lyx; LyxHex; LyxHexOMe; LyxOMe; LyxOctUlo-onic; Lyxf; Man; Man-ol; ManA; ManCMe; ManF; ManGroP; ManN; ManNAc; ManNAcA; ManNAcAAla; ManNAcAGro; ManNAcAN; ManNAcANOOrn; ManNAcASer; ManNAcAThr; ManNAcGroA; ManNAcGroP; ManNAcGroPA; ManNAcNAmA; ManNAcNEtnA; ManNAcOAc; ManNAcOAcA; ManNAcOLac; ManNAcOMe; ManNAcOMeAN; ManNAcOPEtn; ManNAcOPyr; ManNBut; ManNGroP; ManNonUloNAc-onic; ManOAc; ManOAcA; ManOAcN; ManOAcOMe; ManOAcOPyr; ManOAep; ManOBut; ManOEtn; ManOLac; ManOMe; ManOMeA; ManOP; ManOP-ol; ManOPCho; ManOPEtn; ManOPOMe; ManOPOPyr-ol; ManOPy; ManOPyr; ManOS; ManOctUlo; ManSH; Manf; Mur; MurNAc; MurNAcAla; MurNAcOP; MurNAcSer; Neu; Neu5Ac; Neu5AcN; Neu5AcNAc; Neu5AcNMe; Neu5AcOAc; Neu5AcOAcOMe; Neu5AcOGc; Neu5AcOMe; Neu5AcOS; Neu5Gc; Neu5GcA; Neu5GcN; Neu5GcOMe; Neu5GcOS; NeuOFo; NeuOMe; OLac; Ole; Oli; OliN; OliNAc; OliOMe; Par; Parf; PerNAc; Pse; Pse5Ac7Ac; Pse5Ac7AcNBut; Pse5Ac7AcOBut; PseNAc; PseNAcNAm; PseNAcNBut; PseNAcNFo; PseNAcNGro; PseNAcOAcNBut; PseNAcOBut; PseNButNFo; PseNGcNAm; PseOAc; PseOAcOFo; PseOFo; Qui; QuiN; QuiNAc; QuiNAc-ol; QuiNAcGro; QuiNAcGroP; QuiNAcN; QuiNAcNAlaAc; QuiNAcNAm; QuiNAcNAspAc; QuiNAcNBut; QuiNAcNButGro; QuiNAcNGroA; QuiNAcOAc; QuiNAcOBut; QuiNAcOMe; QuiNAcOP; QuiNAla; QuiNAlaAc; QuiNAlaAcGro; QuiNAlaBut; QuiNAlaButGro; QuiNAspAc; QuiNBut; QuiNButAla; QuiNButOMe; QuiNFo; QuiNGlyAc; QuiNHse; QuiNHseGro; QuiNLac; QuiNMal; QuiNSerAc; QuiNThrAc; QuiOMe; QuiOMeN; QuiOS; QuiOSN; QuiOSNBut; Rha; Rha-ol; RhaCMe; RhaCl; RhaGro; RhaGroA; RhaGroP; RhaNAc; RhaNAcNBut; RhaNAcNFo; RhaNAcOAc; RhaNPro; RhaOAc; RhaOAcOLac; RhaOAcOMe; RhaOBut; RhaOFer; RhaOLac; RhaOMe; RhaOMeCMeNLac; RhaOMeCMeOFo; RhaOP; RhaOPEtn; RhaOPOMe; RhaOProp; RhaOPyr; RhaOS; Rhaf; Rib; Rib-ol; RibGroP-ol; RibOAc; RibOAcOP-ol; RibOP-ol; RibOPEtn-ol; RibOPGroP-ol; Ribf; Ribf-uronic; RibfOAc; Sed; Sedf; Sor; Sorf; Suc; Sug; SugOAc; Tag; Tal; The; Thr; Thre-ol; Thre-onic; Tyv; VioNAc; Xluf; XlufOMe; Xyl; Xyl-ol; Xyl-onic; XylHex; XylHexNAc; XylHexUlo; XylHexUloN; XylHexUloNAc; XylNAc; XylNMe; XylOAc; XylOBz; XylOMe; XylOP; XylOS; Xylf; Yer; YerOAc; a-Tri-ol; a-Tri-onic; aldehyde-2,5-Anhydro-L-Man; aldehyde-2,5-Anhydro-Tal; aldehyde-Gro; aldehyde-Hex; aldehyde-L-Gro; aldehyde-L-GroN; aldehyde-QuiNAc; aldehyde-Rib; aldehyde-a-Tri-ol; aldehyde-b-Tri-ol; b-Tri-N-ol; b-Tri-OP-ol; b-Tri-ol; b-Tri-onic; b1-4Glc; cNeu5Ac;
b) and from monomers listed in FIG. 6,7 and the like, and chemical derivatives thereof;
c) deoxysugars of (a), (b) (Deoxyribose, Rhamnose, Fuculose, Fucose, and the like);
d) uronic acids of (a), (b), (c)—(glucuronic acid, iduronic acid, etc), which may be methoxylated, ethoxylated or acetylated;
e) (a), (b), (c), (d) in which a hydroxyl group has been replaced with an amine group—amino sugars (galactosamine, glucosamine, sialic acid, N-acetylglucosamine, N-acetylgalactosamine, N-acetylneuraminic acid and the like (more then 60 aminosugars));
f) sulfonic acid derivatives of (a), (b), (c), (d), (e)—sulfosugars (O-, N-sulfated, and the like);
g) acetyl; Anhydro, D-alanyl; N-acetyl-D-alanyl; N-acetimidoyl; N—(N-methyl-acetimidoyl); N—(N,N-dimethyl-acetimidoyl); formyl; glycolyl; N-acetyl-glutaminyl; N-methyl-5-glutamyl; glycyl; glyceryl; 2,3-di-O-methyl-glyceryl; 4-hydroxybutyryl; 3,4-dihydroxybutyryl; (R)-3-hydroxybutyryl; (S)-3-hydroxybutyryl; lactyl; methyl; amino; N-acetyl; phosphate; pyruvyl; 1-carboxyethylidene; sulfate; tauryl; Me; Et; Pr; Bu; Pe; Hx; Hp; Oc; Nn; Dec; Und; Dod; Acyl; RCO—; Fo (or HCO); Ac; Gc; Pp; Br (Butyryl); VI (valeryl); Hxo; Hpo; Oco; Nno; Dco; Udo; Lau; Myr; Pam; Ste; eSte; Lin; D4Ach; Gro; Gra; Grn; Gri; Ose; Glcd; Gal; Fuc; GlcA; GlcUe; GlcN; GlcNAc; Neu; Sia; Mur; NeuAcg; NeuGc; Cer; Cho; Etnh; Ins; Ser; Ptd; Sph; Spd; P (Phosphoric residue) derivatives of (a), (b), (c), (d), (e), (f);
h) (a), (b), (c), (d), (e), (f), (g) salts (gum, gummi, and the like);
i) compounds of formula: 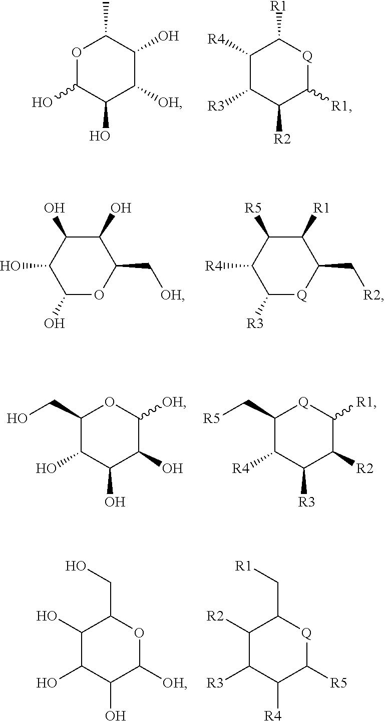
and structural isomers, anomers thereof; and stereoisomers thereof; and the like;
wherein R1-R5 is H, halogen, F, Cl, Br, I, OH, Me, Et, Ac, OMe, OEt, OAc, OR^{A}, N(R^{A})₂, N₃, CN, NO₂, S(O)₇R^{A}, (C₁-C₁₅)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, —C(═O)R^{A}, —C(═O)OR^{A}, —C(═O)NR^{A}R^{B}, —C(═O)SR^{A}, —S(O)R^{A}, —S(O)₂R^{A}, —S(O)(OR^{A}), —S(O)₂ (OR^{A}), —SO₂NR^{A}R^{B}, (C₆-C₂₀)aryl(C₁-C₈)alkyl, —PO(OH)₂, —PO(OH)OPO(OH)₂, —PO(OH)OPO(OH)O PO(OH)₂, —PO(OR^{A})₂, —POR^{A}R^{B}, phenyl, 1-naphthyl, 2-naphthyl, benzyl, (C₃-C₆)cycloalkyl, —CH₂—(C₃-C₆)cycloalkyl, —O—(C₁-C₈) alkyl, —O-benzyl, —O—CH₂—(C₃-C₆)cycloalkyl, CF₃, C(halogen)₃, —C(=Q)R^{A}, —C(=Q)OR^{A}, —C(=Q)N(R^{A}), —NR^{A}R^{B}, —⁺N(R^{A})₃, —SR^{A}, —S(O)R^{A}, —S(O)₂R^{A}, —S(O)(OR^{A}), —S(O)₂(OR^{A}), —OC(=Q)R^{A}, —OC(=Q)OR^{A}, —OC(=Q)(N(R^{A})₂), —SC(=Q)R^{A}, —SC(=Q)OR^{A}, —SC(=Q)(N(R^{A})₂), —N(R^{A})C(=Q)R^{A}, —N(R^{A})C(=Q)OR^{A}, —N(R^{A})C(=Q)N(R^{A})₂, —SO₂NR^{A}₂, —CN, —N₃, —NO₂, —OR^{A}, NR^{A}R^{B}, N(R^{A})OR^{B}, NR^{A}NR^{B}, N₃, NO, NO₂, CHO, CN, —CH(═NR^{A}), —CH═NNHR^{A}, —CH═N(OR^{A}), —CH(OR^{A})₂, —C(═O)NR^{B}R^{A}, —C(═S)NR^{B}R^{A}, —C(═O)OR^{A}, (C₆-C₂₀)optionally substituted aryl, optionally substituted heteroaryl, —C(═O)(C₁-C₈)alkyl, —S(O)_{z}(C₁-C₈)alkyl, SR^{A}, or selected from radicals obtained by removal of a hydrogen atom from the compounds shown in FIG. 8;
wherein Q is O, S, NR^{A}, ⁺N(O)(R^{A}), N(OR^{A}), ⁺N(O)(OR^{A}), or N—NR^{A}₂;
wherein R^{A}, R^{B} is H, halogen, F, Cl, Br, I, OH, Et, Me, Ac, OR^{C}, N(R^{C})₂, N₃, CN, NO₂, S(O)₇R^{C}, (C₁-C₁₅)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, —C(═O)R^{C}, —C(═O)OR^{C}, —C(═O)NR^{C}R^{C}, —C(═O)SR^{C}, —S(O)R^{C}, —S(O)₂R^{C}, —S(O)(OR^{C}), —S(O)₂ (OR^{C}), —SO₂NR^{C}R^{C}, (C₆-C₂₀)aryl(C₁-C₈)alkyl, —PO(OH)₂, —PO(OH)OPO(OH)₂, —PO(OH)OPO(OH)OPO(OH)₂, —PO(OR^{C})₂, —POR^{C}R^{C}, phenyl, 1-naphthyl, 2-naphthyl, benzyl, (C₃-C₆)cycloalkyl, —CH₂—(C₃-C₆)cycloalkyl, —O—(C₁-C₈) alkyl, —O-benzyl, —O—CH₂—(C₃-C₆)cycloalkyl, CF₃, C(halogen)₃, —C(O) R^{C}, —C(O)OR^{C}, —C(O)N(R^{C}), NR^{C}R^{C}, —⁺N(R^{C})₃, —SR^{C}, —S(O) R^{C}, —S(O)₂R^{C}, —S(O)(OR^{C}), —S(O)₂(OR^{C}), OC(O) R^{C}, —OC(O)OR^{C}, —OC(O)(N(R^{C})₂), —SC(=Q)R^{C}, —SC(=Q)OR^{C}, —SC(=Q)(N(R^{C})₂), —N(R^{C})C(=Q)R^{C}, —N(R^{C})C(=Q)OR^{C}, —N(R^{C})C(=Q)N(R^{C})₂, —SO₂NR^{C}₂, —CN, —N₃, —NO₂, —OR^{C}, NR^{C}R^{C}, N(R^{C})OR^{C}, NR^{C}NR^{C}, N₃, NO, NO₂, CHO, CN, —CH(═NR^{C}), —CH═NNHR^{C}, —CH═N(OR^{C}), —CH(OR^{C})₂, —C(═O)NR^{C}R^{C}, —C(═S)NR^{C}R^{C}, —C(═O)OR^{C}, (C₆-C₂₀)optionally substituted aryl, optionally substituted heteroaryl, —C(═O)(C₁-C₈)alkyl, —S(O)_{z}(C₁-C₈)alkyl, or SR^{C};
wherein R^{C} is H, OH, F, Cl, Br, I, Me, Et, Ac, OMe, OEt, or OAc, or selected from radicals obtained by removal of a hydrogen atom from the compounds shown in FIG. 8;
wherein z is from 1 to 10;
and structural isomers thereof; and stereoisomers thereof; and the like;
D2) glycated (O-, N-, P-, C-, S-linked, and the like) derivatives of (D1);
D3) (D1) or (D2) binded (O-, N-, P-, C-, S-linked, and the like) to any Protein, Peptide, Aptamer, Antibodie, Affibody, Avimer or Nanobody;
and wherein bonds may be covalent bonds, α1-1, α1-2, α1-3, α1-4, α1-5, α1-6, α1-7, α1-8, α2-1, α2-2, α2-3, α2-4, α2-5, α2-6, α2-7, α2-8, α2-9, α6-6, β1-1, β1-2, β1-3, β1-4, β1-5, β1-6, β1-7, β1-8, β1-9, β2-1, β2-2, β2-3, β2-4, β2-5, β2-6, β2-7, β2-8, β3-3 α1-2, and the like, αq-w, βq-w wherein q,w from 1 to 9;
E) wherein [HOST]ₙ is[X]ₙ wherein X is selected from: 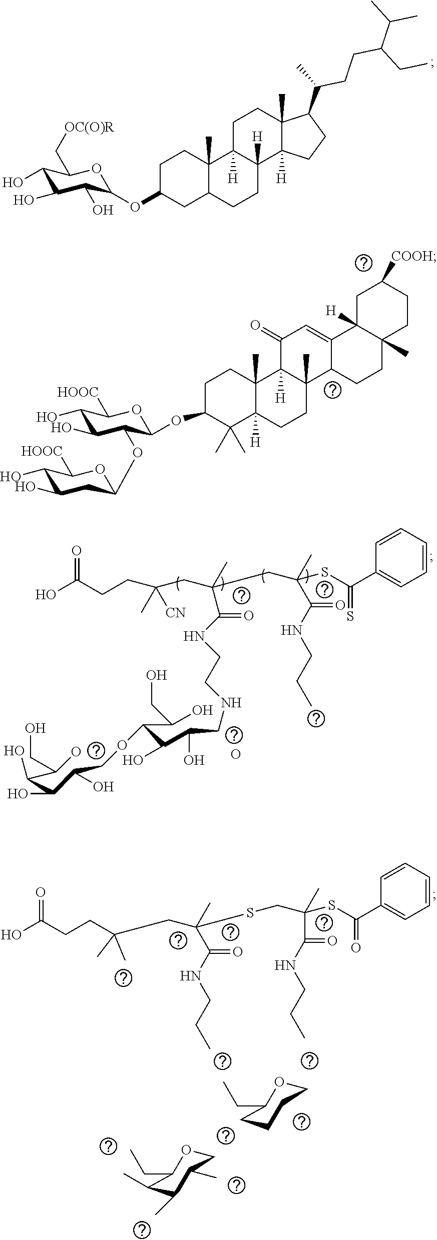 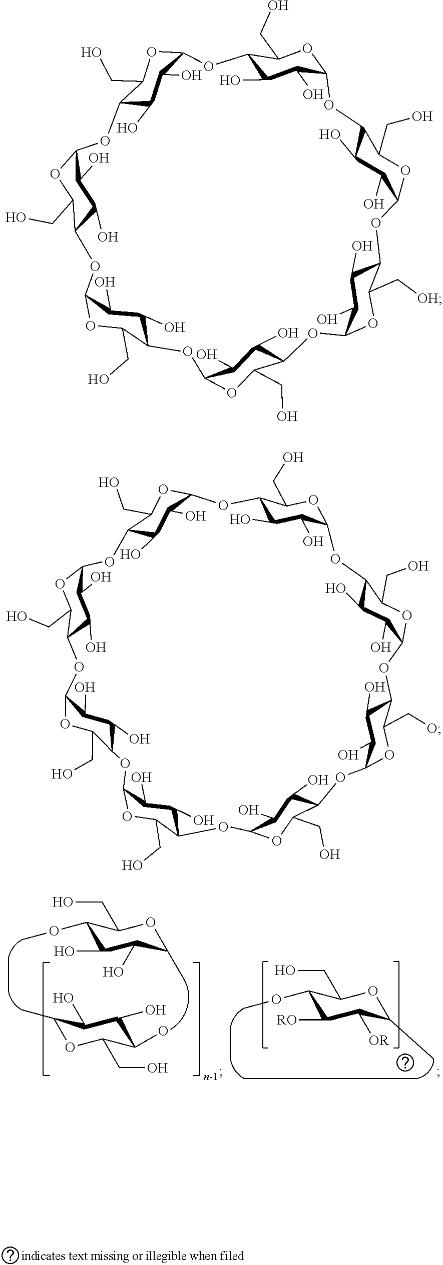
wherein R described in [0091] (B).
F) glycated derivatives, chemical or physical derivatives of A-E, structural isomers thereof; and stereoisomers thereof; and the like.

2. The methods and the complex of claim 1
wherein GUEST is 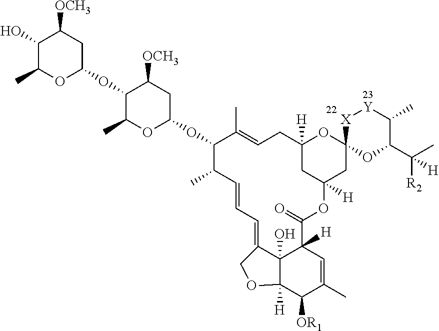
wherein
1) R₁ is CH₃; R₂ is CH₂CH₃; X—Y is CH═CH; (i.e. GUEST is Avermectin A1a);
2) R₁ is CH₃; R₂ is CH₃; X—Y is CH═CH; (i.e. GUEST is Avermectin A1b);
3) R₁ is CH₃; R₂ is CH₂CH₃; X—Y is CH₂—CHOH; (i.e. GUEST is Avermectin A2a);
4) R₁ is CH₃; R₂ is CH₃; X—Y is CH₂—CHOH; (i.e. GUEST is Avermectin A2b);
5) R₁ is H; R₂ is CH₂CH₃; X—Y is CH═CH; (i.e. GUEST is Avermectin B1a);
6) R₁ is H; R₂ is CH₃; X—Y is CH═CH; (i.e. GUEST is Avermectin B1b);
7) R₁ is H; R₂ is CH₂CH₃; X—Y is CH₂—CHOH; (i.e. GUEST is Avermectin B2a);
8) R₁ is H; R₂ is CH₃; X—Y is CH₂—CHOH; (i.e. GUEST is Avermectin B2b);
9) R₁ is H; R₂ is CH₂CH₃; X—Y is CH₂—CH₂; (i.e. GUEST is Ivermectin B1a);
10) R₁ is H; R₂ is CH₃; X—Y is CH₂—CH₂; (i.e. GUEST is Ivermectin B1b);
11) R₁ is CH₃; R₂ is CH₂CH₃; X—Y is CH₂—CH₂; (i.e. GUEST is Ivermectin A1a);
12) R₁ is CH₃; R₂ is CH₃; X—Y is CH₂—CH₂; (i.e. GUEST is Ivermectin A1b);
But excluding combinations with cyclodextrins in the treatment of cancer (at a dosage of approximately 20 to 400 micrograms per kilogram), but not excluded in the treatment of non-cancerous diseases.

3. The methods and the complex of claim 1 or 2 wherein carrier for non-covalent active pharmaceutical ingredient-carrier complex is selected from:
A. Polymers and oligomers, predominantly organic polymers and oligomers, predominantly water soluble organic polymers and oligomers, natural polymers and oligomers, biopolymers and biooligomers even more predominantly polysaccharides, homopolysaccharides, heteropolysaccharides, oligosaccharides, glycoconjugates, glycoproteins (O-, N-, P-, C-, S-linked), neoglycoproteins, glycolipids, glycopeptides, proteoglycans, peptidoglycans, glycolipids, glycosides, lipopolysaccharides, hemicelluloses, storage polysaccharides, sulfated polysaccharides and oligosaccharides, pectins, gums, mucilages, which may be (but are not exclusive to):
A.1. Polyelectrolites and non-polyelectrolites;
A.2. Polymers and oligomers, which can be obtained from animal, nonanimal, natural (i.e., microbiological or plant) sources:
a) from plants, higher plants (non-limiting examples): Starch; Cellulose; Guar gum; Gum arable; Locust beanvgum; Pectin; Acacia gum; Gum arabic; Arabinogalactan; Costus glucans; Xylan; Glucomannan; Xyloglucan; Inulin; beta-glucan; Guar gum; Karaya gum; Tara gum; Fenugreek gum; Locust bean gum; Tragacanth gum; Cassia tor a gum; and the like;
b) from mushrooms (non-limiting examples): Polysaccharide-K (Krestin, PSK), beta-glucans, Polysaccharide peptide (PSP); and the like;
c) from Algae (non-limiting examples): Alginates; Galactans; Carrageenan; Alginate; Agar; Carrageenan; Fucoidan; Ulvan; Laminarin; Angelan; Porphyran; Spirulan; Agarose; Rhodymenan; and the like;
d) from animal origin (non-limiting examples): Glycosaminoglycans(GAGs); Hyaluronic acid; Glycogen; Hyaluronan; Chitin; Chitosan; Heparin; Chondroitin sulphate; Keratan sulphate; Dermatan sulphate; Asialofetuin; fetuin; and the like;
e) from microbial origin (non-limiting examples): Dextran; Gellan gum; Pullulan; Xanthan gum; Curdlan; Scleroglucan; Pullulan; Dextran; Xanthan; Levan; Gellan; Emulsan; Schizophyllan; Glucuronan; Succinoglycan; Nigcran; and the like;
A.3. Hemicelluloses, which can be arabanes, arabinans, galactans (galactosans), glucans, xylans, mannans, fructans, xyloglucans, arabinogalactans, arabinoxylans, glucomannans, galactomannans, galactoglucomannans, beta-glucans, galactogens, and the like, their mixtures, but not excluding other hemicelluloses, and mixtures thereof;
A.4. Sulfated polysaccharides and oligosaccharides which may be fucoidans, carrageenans or carrageenins, agaropectins, sea cucumber sulfated polysaccharides (SCSP), chondroitin sulfate, keratan sulfate, and the like, their mixtures, but not excluding other sulfated polysaccharides and oligosaccharides, and mixtures thereof;
A.5. Oligosaccharides, fructooligosaccharides, galactooligosaccharides (oligogalactosyllactose, oligogalactose, oligolactose, transgalactooligosaccharides), xylooligosaccharides, isomaltooligosaccharides and the like, but not excluding other oligosaccharides and mixtures thereof;
A.6. Polysaccharides and oligosaccharides, storage polysaccharides, sulfated polysaccharides and oligosaccharides, pectins, gums, mucilages which may be (but are not exclusive to others): polyuronic acids and esters; gum(s), gum arabic, xanthan gum, oat gum, gellan gum, guar gum, carob gum, karaya gum, dammar gum, gummiarabica, tara gum, ghatti gum, british gum, agar, agar-agar, tragakant gum, conjac gum, velan gum, dutan gum; galacturonic acid-based polysaccharides with side chains of rhamnose, arabinose, xylose and fructose and their salts (pectins, pectates, pectinates); pectin, pectins of beets, carrots, peppers, pumpkins, eggplant, sunflower, apples, quinces, cherries, plums, pears, citrus, zosterin; modified pectins, modified citrus pectin; acidic polysaccharides—i.e. e. polysaccharides containing carboxyl groups and/or phosphate groups and/or sulfuric ester groups; plantain husk, psyllium; soybean hemicellulose; galacturones, homogalacturones, polygalacturonic acids and their salts, rhamnogalacturonan, rhamnogalactans; calloses, laminarins, chrysolaminarins, curdlans; inulins, guars, dextrans, pullulans; agaroses; alginic acid and its salts alginates, propylene glycol alginate; arabin, arabic acid and its salts, and the like, but not excluding other, and mixtures thereof;
A.7. Cellulose, cellulosic polymers, methylcellulose, ethylcellulose, hydroxypropylcellulose(s) (HPC), hydroxypropylmethylcellulose acetate succinate, hypromellose(s), hydroxypropylmethylcellulose(s) (HPMC), methylethylcellulose, ethylhydroxyethylcellulose, croscaramellose, carboxymethylcellulose and its salts; starch, starch(es), starch 1500G, soluble starch, modified starches, hydroxyethyl starch, cationic starch, acid-treated starch, alkaline modified starch, bleached starch, oxidized starch, enzyme treated starch, monostarch phosphate, distarch glycerol, distarch phosphate, phosphated distarch phosphate, acetylated distarch phosphate, starch acetate esterified with acetic anhydride, starch acetate esterified with vinyl acetate, acetylated distarch adipate, acetylated distarch glycerol, distarch glycerine, hydroxy propyl starch, hydroxy propyl distarch glycerine, hydroxy propyl distarch phosphate, hydroxy propyl distarch glycerol, starch sodium octenyl succinate, acetylated oxidised starch, and the like;
A.8. Dextrin(s), maltodextrins, amylodextrins, polydextroses; amylopectin, amylose, glycogen; chitosan, chitins; pullulans, glucuronoaraboxylans, methyl-glucuronoaraboxylans, glycosaminoglycans, mucopolysaccharides, heparin/heparan sulfate, chondroitin sulfate, dermatan sulfate, keratan sulfate, hyaluronan, hyaluronic acid, and the like; and mixtures thereof;
A.9. Glycated derivatives of these polymers and oligomers (O-, N-, P-, C-, S-linked);
but not excluding other polymers and oligomers, biopolymers and biooligomers even more predominantly polysaccharides, homopolysaccharides, heteropolysaccharides, oligosaccharides, glycoconjugates, glycoproteins (O-, N-, P-, C-, S-linked), neoglycoproteins, glycolipids, glycopeptides, proteoglycans, peptidoglycans, glycolipids, glycosides, lipopolysaccharides, hemicelluloses, storage polysaccharides, sulfated polysaccharides and oligosaccharides, pectins, gums, mucilages and mixtures thereof;
B. Polymers, syntetic polymers, predominantly water-soluble polymers which may be (but are not limited to): polymers and copolymers formed from acrylic acid, methacrylic acid, and/or esters thereof; polymethacrylates, Eudragit and their salts; polyacrylamides; poly(amidoamine)s,
poly(propyleneimine)s, polyethylene glycols; polyvinyl alcohol; polyvinylpyrrolidone; acrylic polymers, cellulose acetate phthalate(s), copovidone(s), ethyl oleate, glycerol derivatives, glyceryl triacetate, polyethylene glycol(s) (PEG), PEG derivatives, polymethacrylates, propylene glycol, propylene glycol derivatives, povidone(s), polyvinylpyrrolidone(s) (PVP), polyvinyl acetate phthalate(s) (PVAP), hypromellose acetate succinate(s) (HPMCAS), hydroxypropyl methylcellulose acetate succinate (HPMCAS), hypromellose phthalate(s) (HPMCP), cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, Avicel(s), Avicel PH101, Avicel PH102, Benecel(s), Brij(s), Brij 30, Brij 35, Capryol(s), Cavamax(s), Cavasol(s) and Cavitron(s) HPpCD cyclodextrins, Compritol 888 ATO, Cremophor(s), Cremophor EL, Cremophor RH40, DiCal Dihydrate, epoxidized palm oil (Epo), Eudragit(s), Eudragit E, Eudragit EPO, Eudragit L100, Eudragit L100-55, Eudragit S100, Gelucire(s), Gelucire 44/14, HP-50 AAS-LF, HP-55 AAS-MF, HPMC(p-606), HPMC-E, HPMC-F, HPMC-K, HPMCAS-H, HPMCAS-L, HPMCAS-M, HPMCAS SDD, HPMCAS(AS-MG), HPMCAS-M SDDs, HPMCAS-MG, HPMCP (HP 55), HPMCP-HP55, HPMCPh, hypromellose phthalate HP-50, Imwitor(s), Imwitor 742, Klucel HPC, Kolhdon 17 PF, Kollicoat(s), Kollicoat IR, Kollicoat MAE, Kollicoat MAE 100, Kollicoat MAE 100P, Kollicoat Protect, Kollidon(s) (povidone(s)), Kollidon 12 pf, Kollidon 12/17PF, Kollidon 30, Kollidon 30/90, Kollidon 90, Kollidon CL-F, Kollidon CL-SF, Kollidon K30, Kollidon SR, Kollidon SR, Kollidon SR(PVAc), Kollidon V64/Fine, Kollidon VA 64, Kollidon VA 64 (copovidone), Kollidon VA64, Kolliphor(s), Kolliphor EL, Kolliphor EL/ELP, Kolliphor HS15, Kolliphor P 188, Kolliphor P 188/407, Kolliphor P 188/micro, Kolliphor P 407, Kolliphor P 407/micro, Kolliphor PS 20, Kolliphor PS 60, Kolliphor PS 80, Kolliphor RH 40, Kolliphor SLS, Kolliphor SLS/fine, Kollisolv(s), Kollisolv GTA, Kollisolv PEG 1450, Kollisolv PEG 300, Kollisolv PEG 3350, Kollisolv PEG 400, Kollisolv PEG E 300, Kollisolv PEG E 400, Kollisolv PEG grades (polyethylene glycol), Kolliwax(s), Kolliwax GMS II, Kolliwax SA, Labrasol(s), Lactose 310 Mono, Lactose FF316, Laurogucol(s), Maisine(s), Miglyol(s), Myrj(s), Myrj 52, PEG 1000, PEG 10000, PEG 1500, PEG 2000, PEG 20000, PEG 3000, PEG 400, PEG 4000, PEG 600, PEG 6000, PEG 800, PEG 8000, Pharmacoat(s), PVP K-12, PVP K-120, PVP K-15, PVP K-17, PVP K-30, PVP K-60, PVP K-90, PVP SDD, PVP VA64 SDDs, PVP-VA, PVP-VA 64, PVP-VA SDD, Palm stearin based polyesteramide (PSPEA), Peceol(s), pectin(s), Plasdone(s), Plasdone K povidone, Plasdone K-12 povidone, Plasdone K-29/32 povidone, Plasdone K-90 povidone, Plasdone S, Plasdone S-630 copovidone, poly(2-ethyl-2-oxazoline), poly(ethylene oxide) (PEO) (3400, 10000, 20000), polyoxyethylene stearate, Shin-Etsu AQOAT(s), Soluplus(es), Solutol(s), sucrose laurate, tocopheryl PEG 1000-succinate (TPGS), vitamin E TPGS, d-α-tocopherol polyethylene glycol 1000 succinate (TPGS), Isomalt (Galen IQ 810), galactosylated Polymers; galactosylated ciclodextrins; cationic glycopolymers; block and statistical carbohydrate-based galactosylated copolymers; galactosylated albumin(s), galactosylated bovine serum albumin; Statistical Cationic Glycopolymer of 2-aminoethyl methacrylamide (AEMA) and 2-lactobionamidoethyl methacrylamide (LAEMA) P(AEMA n-st-LAEMA m) (m,n from 1 to 1000); Cationic Block Glycopolymer of 2-aminoethyl methacrylamide (AEMA) and 2-lactobionamidoethyl methacrylamide; p-Vinylbenzyl galactoside (VBG); galactosylated N-3-guanidinopropyl methacrylamide-co-poly (ethylene glycol) methacrylate copolymers; galactosylated amphiphilic graft copolymer (PHEA-g-BIB-pButMA-g-PEG-GAL); (cholesteryloxycarbonylamino) ethylamine-α,β-polyasparthydrazied (CHE-PAHy-Lacs); PLGA-di-GAL; and the like, and others which are described in the scientific and patent literature (for
example: Macro-Glycoligands. Methods and Protocols, Editors: Xue-Long Sun); and the like; but not excluding other synthetic polymers and mixtures thereof;
C. Copolymers is made from monomers of the polymers listed in paragraph A, B including alternating copolymers, random copolymers, block copolymers, graft copolymers, cross-linked modifications which may be (but are not limited to): methacrylic acid and ethyl acrylate copolymers; methacrylic acid copolymers; vinylpyrrolidone-vinyl acetate copolymers (PVPVA); polyvinylpolypyrrolidone (PVPP); PVA-PEG graft co-polymers; HPMC and PVA-PEG grafted copolymers; polyvinylpyrrolidone-arabinogalactan copolymers; grafted polyvinylpyrrolidone-arabinogalactan copolymers; the graft copolymer has a) poly(vinyl acetate) and/or poly (vinyl alcohol) and/or poly(vinyl chloride) and poly(vinyl ester) on b) a polymer chain of polyethylene glycols, polyalkylene glycols, polypropylene glycols, polyisobutylene glycols or polymethylpentene glycols; graft copolymer polyvinyl acetate and/or hydrolysed polyvinyl acetate (polyvinyl alcohol) groups on a polyalkylene oxide (preferably polyethylene oxide); vinylpyrrolidone-vinyl acetate copolymers; vinylpyrrolidone-vinyl acetate copolymer-64+; vinylpyrrolidone-vinyl acetate VA 64; polymethacrylate-based copolymers includes anionic, cationic, and neutral copolymers based on methacrylic acid and methacrylic/acrylic esters their salts, esters or other derivatives and mixtures thereof and the like;
D. Polymers and oligomers, copolymers, biopolymers (natural, obtained by chemical or physical modification of natural polymers (chemical or physical derivatives), or synthesized specifically) that under at least one of the following conditions:
1. co-occurring in physiologic body fluids, plasma, intercellular fluid together with APIs;
2. by non-covalent binding to API;
3. by covalent binding to API into a conjugate;
result in at least one of the following:
a. an increase in the permeability of the API, API-carrier system (solution, non-covalent complex, conjugate, and the like);
b. an increase in the concentration of API, API-carrier system in cells and tissues;
for at least one of the following reasons:
i. inhibition or modulation of multidrug resistance (MDR), predominantly mediated by efflux transporters of the ATP-binding cassette (ABC) superfamily including P-glycoprotein (P-gp);
ii. induction, inhibition or modulation of CYP1, CYP2, CYP3 enzymes (non limiting examples: CYP1A1, 1A2, 2A6, 2B6, 2C8, 2C9, 2C19, 2D6, 2E1 or 3A4 enzymes);
iii. due to a change in the structure of the cell membrane, change in lipid biolayer properties after the incorporation of carrier substance molecules (or carrier molecular part of NCC or conjugate) into this membrane or due adhesion to this membrane;
iv. due to changes in charge, hydrophilicity, hydrophobicity or other biophysical properties of the non-covalent complex, or conjugate due to which changes in the transmembrane (passive, active) or paracellular transport of the non-covalent complex, or conjugates;
v. for other reasons e.g., by the introduction or presence in the carrier of targeted fragments capable of selectively interacting with the receptor, cell surface receptors, membrane receptors, transmembrane receptors. Low molecular weight compounds, peptides, aptomers, and the like are used as vector molecules and are described in the patent or scientific literature; or by other reasons described in the patent or scientific literature.
E. Other carriers:
E1. Macrocyclic hosts which may be (but are not exclusive to others): cyclodextrins, cucurbit[n]urils, and calix[n]arenes, pillarenes, crown ethers, cyclophanes, cryptands;
E2. Acids and salts based on these acids which may be (but are not limited to): citric acid, tartaric acid, succinic acid, phosphoric acid, aminoacids, acetate(s), sodium acetate, alginate(s), sodium alginate, glycyrrhizic acid and their salts, and mixtures thereof; 8
E3. Polyols which may be (but are not limited to): ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and mixtures thereof;
E4. Monosaccharides, disaccharides, trisaccharides, tetrasaccharides, pentaccharides, hectaccharides and the like mixtures thereof;
E5. Surfactants which may be (but are not limited to): anionic surfactants contained anionic functional groups at their head, such as sulfate, sulfonate, phosphate, carboxylates, carboxylate salts; alkyl sulfates; alkyl-ether sulfates; carboxylate-based fluorosurfactants; cationic surfactants; primary, secondary, or tertiary amines; permanently charged quaternary ammonium salts; zwitterionic (amphoteric) surfactants; zwitterionic surfactants which cationic part is based on primary, secondary, or tertiary amines or quaternary ammonium cations; sultaines; betaines; phospholipids; zwitterionic surfactants of the tertiary amine oxides structural type; non-ionic surfactants; ethoxylates; fatty alcohol ethoxylates; alkylphenol ethoxylates (apes or apeos); fatty acid ethoxylates; special ethoxylated fatty esters and oils; ethoxylated amines and/or fatty acid amides; terminally blocked ethoxylates; fatty acid esters of polyhydroxy compounds; fatty acid esters of glycerol; fatty acid esters of sorbitol; fatty acid esters of sucrose; alkyl polyglucosides; ammonium lauryl sulfate; sodium lauryl sulfate; sodium dodecyl sulfate; sodium laureth sulfate; sodium lauryl ether sulfate; sodium myreth sulfate; docusate (dioctyl sodium sulfosuccinate) and their salts; perfluorooctanesulfonate (pfos); perfluorobutanesulfonate; alkyl-aryl ether phosphates; alkyl ether phosphates; sodium stearate; sodium lauroyl sarcosinate; perfluorononanoate; perfluorooctanoate; octenidine dihydrochloride; cetrimonium bromide (ctab); cetylpyridinium chloride (cpc); benzalkonium chloride (bac); benzethonium chloride (bzt); dimethyldioctadecylammonium chloride; dioctadecyldimethylammonium bromide (dodab); chaps (3-[(3-cholamidopropyl)dimethylammonio]-1-propanesulfonate); cocamidopropyl hydroxysultaine; cocamidopropyl betaine; phosphatidylserine; phosphatidylethanolamine; phosphatidylcholine; sphingomyelins; lauryldimethylamine oxide; myristamine oxide; narrow-range ethoxylates; octaethylene glycol monododecyl ether; pentaethylene glycol monododecyl ether; nonoxynols; triton x-100; polyethoxylated tallow amine; cocamide monoethanolamine; cocamide diethanolamine; poloxamers; glycerol monostearate; glycerol monolaurate; sorbitan monolaurate; sorbitan monostearate; sorbitan tristearate; tween(s), tween 20; tween 40; tween 60; tween 80; decyl glucoside; span(s), span 20, span 40, span 80; lauryl glucoside; octyl glucoside; poloxamer(s), poloxamer 188, poloxamer 407, polyoxyethylene stearate, myrj 52, deoxycholic acid, bile acids, pluronic(s), pluronic F-127, pluronic P85, pluronic f68, gelucire(s), gelucire 44/14, lecithins, polysorbates, polysorbate 80, plasdone-s630, pluronic-f68, inutec sp1, compritol 888 ato, tocopherol polyethylene glycol succinate, polyoxyethylated castor oil, polyoxyethylated glycerides, lauroyl macroglycerides, and mono- and di-fatty acid esters of low molecular weight polyethylene glycols; and mixtures thereof;
F. Methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in A-E, and their esters, salts, and any other chemical derivatives, and mixtures thereof or/and which are described in the scientific and patent literature; Alternatively, dextran or poly-L-lysine can be attached to the gum arabic carrier to provide an increased number of sites of attachment for the therapeutic agent.
G. Any combination of substances specified in items A-F.

4. The methods and the complex of claims 1-3 when non-covalent complex when dissolved in a physiologically acceptable solvent other than water.

5. The methods and the complex of claims 1-4 when non-covalent complex is inclusion complex or supramolecular complex.

6. The methods (co-treating methods/solid dispersion techniques) for modifying the properties of an active pharmaceutical ingredient (API) by creating a solid phase inclusion complex or molecular/solid dispersion (both of Formula II) using API, host substance (and, if necessary, other compounds) with properties of increased bioavailability and/or permeability and using Formula II compounds obtained by this methods (or aqueous solutions or suspensions thereof) to treat a cancer in humans and/or to manufacture a pharmaceutical dosage form to treat cancer in a human in need thereof (by administering a therapeutically effective amount)), at a dosage ranging from 0.0001 ng/kg to 100000 mg/kg (calculated for pure API)
wherein active pharmaceutical ingredient is selected from:
Milbemycins; Milbemycin A3; Milbemycin A4; Milbemycin D; Milbemycin B2; Milbemycin B3; Milbemycin G; Nemadectins; Nemadectin; Meilingmycins; Meilingmycin A; Milbemectins; Milbemectin; Milbemycin oxime; Moxidectins; Moxidectin; Doramectins; Doramectin; Selamectins; Selamectin; Eprinomectins; Eprinomectin B1a; Eprinomectin B1b; Emamectins; Emamectin B1a; Emamectin B1b; Tenvermectins; Tenvermectin B; Tenvermectin A; Avermectins; Avermectin A1a; Avermectin A1b; Avermectin A2a; Avermectin A2b; Avermectin B1a; Avermectin B1b; Avermectin B2a; Avermectin B2b; Ivermectins; Ivermectin B1a; Ivermectin B1b; Ivermectin A1a; Ivermectin A1b; Fenbendazole; Flubendazole; Thiabendazole; Oxfendazole; Triclabendazole; Mebendazole; Oxyclozanide; Niclosamide; Nitazoxanide; Colchicine; Artemisinin; Quercetin; Curcumin; Resveratrol; thymoquinone; Pyrvinium; Albendazole sulfoxide; Albendazole; or compounds selected from those listed in from FIG. 12 to FIG. 15; or compounds (API, AAPI, AANPI) selected from those listed in [0110], [0111], [0114]; or derivatives thereof such as racemate, enantiomer, diastereomer, tautomer, polymorph, pseudopolymorph, hydride or solvate thereof; or a pharmaceutically acceptable salt or ester or ether thereof; or prodrug thereof; or methabolite thereof; or aglycone thereof; or derivatives which may be obtained by gene replacement processes in genetically engineered strains, or/and by fermentation, or/and by chemical modifications and the like, which are described in the scientific and patent literature; or mixtures thereof;
wherein the host substance is selected from:
A. Polymers and oligomers, predominantly organic polymers and oligomers, even more predominantly polysaccharides and oligosaccharides, hemicelluloses, storage polysaccharides, sulfated polysaccharides and oligosaccharides, pectins, gums, mucilages, which may be (but are not exclusive to):
A.1. Hemicelluloses, which can be arabanes, arabinans, galactans (galactosans), glucans, xylans, mannans, fructans, xyloglucans, arabinogalactans, arabinoxylans, glucomannans, galactomannans, galactoglucomannans, beta-glucans, galactogens, their mixtures, but not excluding other hemicelluloses, and mixtures thereof;
A.2. Sulfated polysaccharides and oligosaccharides which may be fucoidans, carrageenans or carrageenins, agaropectins, sea cucumber sulfated polysaccharides (SCSP), chondroitin sulfate, keratan sulfate, their mixtures, but not excluding other sulfated polysaccharides and oligosaccharides, and mixtures thereof;
A.3. Polysaccharides and oligosaccharides, storage polysaccharides, sulfated polysaccharides and oligosaccharides, pectins, gums, mucilages which may be (but are not exclusive to others): polysaccharides based on glucose, dextrose, galactose, mannose, arabinose, rhamnose, sucrose, maltose, lactose and their uronic acids, which may be methoxylated or acetylated, and their salts (gum, gummi); polyuronic acids and esters; gum, xanthan gum, oat gum, gellan gum, guar gum, carob gum, karaya gum, dammar gum, gummiarabica, tara gum, ghatti gum, british gum, agar, agar-agar, tragakant gum, conjac gum, velan gum, dutan gum; galacturonic acid-based polysaccharides with side chains of rhamnose, arabinose, xylose and fructose and their salts (pectins, pectates, pectinates); pectin, pectins of beets, carrots, peppers, pumpkins, eggplant, sunflower, apples, quinces, cherries, plums, pears, citrus, zosterin; modified pectins, modified citrus pectin; acidic polysaccharides—i.e. e. polysaccharides containing carboxyl groups, phosphate groups and/or sulfuric ester groups; plantain husk, psyllium; soybean hemicellulose; galacturones, homogalacturones, polygalacturonic acids and their salts, rhamnogalacturonan, rhamnogalactans; calloses, laminarins, chrysolaminarins, curdlans; inulins, guars, dextrans, pullulans; agaroses, galacto-oligosaccharides (oligogalactosyllactose, oligogalactose, oligolactose or transgalactooligosaccharides), xylooligosaccharides, fructooligosaccharides, isomaltooligosaccharide; alginic acid and its salts alginates, propylene glycol alginate; arabin, arabic acid and its salts; cellulose, cellulosic polymers, methylcellulose, ethylcellulose, hydroxypropylcellulose(s) (HPC), hydroxypropylmethylcellulose acetate succinate, hypromellose(s), hydroxypropylmethylcellulose(s), (HPMC), methylethylcellulose, ethylhydroxyethylcellulose, croscaramellose, carboxymethylcellulose and its salts; starch, starch(es), starch 1500G, soluble starch, modified starches, hydroxyethyl starch, cationic starch, acid-treated starch, alkaline modified starch, bleached starch, oxidized starch, enzyme treated starch, monostarch phosphate, distarch glycerol, distarch phosphate, phosphated distarch phosphate, acetylated distarch phosphate, starch acetate esterified with acetic anhydride, starch acetate esterified with vinyl acetate, acetylated distarch adipate, acetylated distarch glycerol, distarch glycerine, hydroxy propyl starch, hydroxy propyl distarch glycerine, hydroxy propyl distarch phosphate, hydroxy propyl distarch glycerol, starch sodium octenyl succinate, acetylated oxidised starch; dextrin(s), maltodextrins, cyclodextrins, amylodextrins, polydextroses; amylopectin, amylose, glycogen; chitosan, chitins; pullulans, glucuronoaraboxylans, methyl-glucuronoaraboxylans, glycosaminoglycans, mucopolysaccharides, heparin/heparan sulfate, chondroitin sulfate, dermatan sulfate, keratan sulfate, hyaluronan, hyaluronic acid and mixtures thereof, but not excluding other polysaccharides and oligosaccharides, hemicelluloses, stored polysaccharides, sulfated polysaccharides and oligosaccharides, mucilages and mixtures thereof;
A.4. Macrocyclic hosts which may be (but are not exclusive to others): cyclodextrins, cucurbit[n]urils, and calix[n]arenes, pillarenes, crown ethers, cyclophanes, cryptands;
B. Substances that may contain in significant amounts (more than 5% wt.) polysaccharides and oligosaccharides, hemicelluloses, storage polysaccharides, sulfated polysaccharides and oligosaccharides, pectins, gums, mucilages, which may be (but are not limited to) plants or algae or animal or fungi, parts of plants or algae or animal or fungi, processed plants or algae or animal or fungi, processed parts of plants or algae or animal or fungi containing in significant amounts the substances specified in paragraph A, and mixtures thereof. A frequent
but non-limiting example is dried brown or red algae such as kelp or focus;
C. Syntetic polymers, predominantly water-soluble polymers which may be (but are not limited to): polymers and copolymers formed from acrylic acid, methacrylic acid, and/or esters thereof; polymethacrylates, Eudragit and their salts; polyacrylamides; poly(amidoamine)s, poly(propyleneimine)s, polyethylene glycols; polyvinyl alcohol; polyvinylpyrrolidone; acrylic polymers, cellulose acetate phthalate(s), copovidone(s), ethyl oleate, glycerol derivatives, glyceryl triacetate, polyethylene glycol(s) (PEG), PEG derivatives, polymethacrylates, propylene glycol, propylene glycol derivatives, povidone(s), polyvinylpyrrolidone(s) (PVP), polyvinyl acetate phthalate(s) (PVAP), hypromellose acetate succinate(s) (HPMCAS), hydroxypropyl methylcellulose acetate succinate (HPMCAS), hypromellose phthalate(s) (HPMCP), cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, Avicel(s), Avicel PH101, Avicel PH102, Benecel(s), Brij(s), Brij 30, Brij 35, Capryol(s), Cavamax(s), Cavasol(s) and Cavitron(s) HPpCD cyclodextrins, Compritol 888 ATO, Cremophor(s), Cremophor EL, Cremophor RH40, DiCal Dihydrate, epoxidized palm oil (Epo), Eudragit(s), Eudragit E, Eudragit EPO, Eudragit L100, Eudragit L100-55, Eudragit S100, Gelucire(s), Gelucire 44/14, HP-50 AAS-LF, HP-55 AAS-MF, HPMC(p-606), HPMC-E, HPMC-F, HPMC-K, HPMCAS-H, HPMCAS-L, HPMCAS-M, HPMCAS SDD, HPMCAS(AS-MG), HPMCAS-M SDDs, HPMCAS-MG, HPMCP (HP 55), HPMCP-HP55, HPMCPh, hypromellose phthalate HP-50, Imwitor(s), Imwitor 742, Klucel HPC, Kolhdon 17 PF, Kollicoat(s), Kollicoat IR, Kollicoat MAE, Kollicoat MAE 100, Kollicoat MAE 100P, Kollicoat Protect, Kollidon(s) (povidone(s)), Kollidon 12 pf, Kollidon 12/17PF, Kollidon 30, Kollidon 30/90, Kollidon 90, Kollidon CL-F, Kollidon CL-SF, Kollidon K30, Kollidon SR, Kollidon SR, Kollidon SR(PVAc), Kollidon V64/Fine, Kollidon VA 64, Kollidon VA 64 (copovidone), Kollidon VA64, Kolliphor(s), Kolliphor EL, Kolliphor EL/ELP, Kolliphor HS15, Kolliphor P 188, Kolliphor P 188/407, Kolliphor P 188/micro, Kolliphor P 407, Kolliphor P 407/micro, Kolliphor PS 20, Kolliphor PS 60, Kolliphor PS 80, Kolliphor RH 40, Kolliphor SLS, Kolliphor SLS/fine, Kollisolv(s), Kollisolv GTA, Kollisolv PEG 1450, Kollisolv PEG 300, Kollisolv PEG 3350, Kollisolv PEG 400, Kollisolv PEG E 300, Kollisolv PEG E 400, Kollisolv PEG grades (polyethylene glycol), Kolliwax(s), Kolliwax GMS II, Kolliwax SA, Labrasol(s), Lactose 310 Mono, Lactose FF316, Laurogucol(s), Maisine(s), Miglyol(s), Myrj(s), Myrj 52, PEG 1000, PEG 10000, PEG 1500, PEG 2000, PEG 20000, PEG 3000, PEG 400, PEG 4000, PEG 600, PEG 6000, PEG 800, PEG 8000, Pharmacoat(s), PVP K-12, PVP K-120, PVP K-15, PVP K-17, PVP K-30, PVP K-60, PVP K-90, PVP SDD, PVP VA64 SDDs, PVP-VA, PVP-VA 64, PVP-VA SDD, Palm stearin based polyesteramide (PSPEA), Peceol(s), pectin(s), Plasdone(s), Plasdone K povidone, Plasdone K-12 povidone, Plasdone K-29/32 povidone, Plasdone K-90 povidone, Plasdone S, Plasdone S-630 copovidone, poly(2-ethyl-2-oxazoline), poly(ethylene oxide) (PEO) (3400, 10000, 20000), polyoxyethylene stearate, Shin-Etsu AQOAT(s), Soluplus(es), Solutol(s), sucrose laurate, tocopheryl PEG 1000-succinate (TPGS), vitamin E TPGS, d-α-tocopherol polyethylene glycol 1000 succinate (TPGS), Isomalt (Galen IQ 810), but not excluding other synthetic polymers and mixtures thereof;
D. Polyols which may be (but are not limited to): ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and mixtures thereof;
E. Saccharides which may be glucose, dextrose, galactose, mannose, arabinose, rhamnose, sucrose, maltose, lactose, ribose, and mixtures thereof;
F. Surfactants which may be (but are not limited to): anionic surfactants contained anionic functional groups at their head, such as sulfate, sulfonate, phosphate, carboxylates, carboxylate salts; alkyl sulfates; alkyl-ether sulfates; carboxylate-based fluorosurfactants; cationic surfactants; primary, secondary, or tertiary amines; permanently charged quaternary ammonium salts; zwitterionic (amphoteric) surfactants; zwitterionic surfactants which cationic part is based on primary, secondary, or tertiary amines or quaternary ammonium cations; sultaines; betaines; phospholipids; zwitterionic surfactants of the tertiary amine oxides structural type; non-ionic surfactants; ethoxylates; fatty alcohol ethoxylates; alkylphenol ethoxylates (apes or apeos); fatty acid ethoxylates; special ethoxylated fatty esters and oils; ethoxylated amines and/or fatty acid amides; terminally blocked ethoxylates; fatty acid esters of polyhydroxy compounds; fatty acid esters of glycerol; fatty acid esters of sorbitol; fatty acid esters of sucrose; alkyl polyglucosides; ammonium lauryl sulfate; sodium lauryl sulfate; sodium dodecyl sulfate; sodium laureth sulfate; sodium lauryl ether sulfate; sodium myreth sulfate; docusate (dioctyl sodium sulfosuccinate) and their salts; perfluorooctanesulfonate (pfos); perfluorobutanesulfonate; alkyl-aryl ether phosphates; alkyl ether phosphates; sodium stearate; sodium lauroyl sarcosinate; perfluorononanoate; perfluorooctanoate; octenidine dihydrochloride; cetrimonium bromide (ctab); cetylpyridinium chloride (cpc); benzalkonium chloride (bac); benzethonium chloride (bzt); dimethyldioctadecylammonium chloride; dioctadecyldimethylammonium bromide (dodab); chaps (3-[(3-cholamidopropyl)dimethylammonio]-1-propanesulfonate); cocamidopropyl hydroxysultaine; cocamidopropyl betaine; phosphatidylserine; phosphatidylethanolamine; phosphatidylcholine; sphingomyelins; lauryldimethylamine oxide; myristamine oxide; narrow-range ethoxylates; octaethylene glycol monododecyl ether; pentaethylene glycol monododecyl ether; nonoxynols; triton x-100; polyethoxylated tallow amine; cocamide monoethanolamine; cocamide diethanolamine; poloxamers; glycerol monostearate; glycerol monolaurate; sorbitan monolaurate; sorbitan monostearate; sorbitan tristearate; tween(s), tween 20; tween 40; tween 60; tween 80; decyl glucoside; span(s), span 20, span 40, span 80; lauryl glucoside; octyl glucoside; poloxamer(s), poloxamer 188, poloxamer 407, polyoxyethylene stearate, myrj 52, deoxycholic acid, bile acids, pluronic(s), pluronic F-127, pluronic P85, pluronic f68, gelucire(s), gelucire 44/14, lecithins, polysorbates, polysorbate 80, plasdone-s630, pluronic-f68, inutec sp1, compritol 888 ato, tocopherol polyethylene glycol succinate, polyoxyethylated castor oil, polyoxyethylated glycerides, lauroyl macroglycerides, and mono- and di-fatty acid esters of low molecular weight polyethylene glycols; and mixtures thereof;
G. Acids and salts based on these acids which may be (but are not limited to): citric acid, tartaric acid, succinic acid, phosphoric acid, aminoacids, acetate(s), sodium acetate, alginate(s), sodium alginate, glycyrrhizic acid and their salts, and mixtures thereof;
H. Oxides and salts based on these oxides which may be (but are not limited to): silicon dioxide, silicates, titanium dioxide, and mixtures thereof;
I. Copolymers of the polymers listed in paragraph A, C including alternating copolymers, random copolymers, block copolymers, graft copolymers, cross-linked modifications which may be (but are not limited to): methacrylic acid and ethyl acrylate copolymers; methacrylic acid copolymers; vinylpyrrolidone-vinyl acetate copolymers (PVPVA); polyvinylpolypyrrolidone (PVPP); PVA-PEG graft co-polymers; HPMC and PVA-PEG grafted copolymers; grafted polyvinylpyrrolidone-arabinogalactan copolymers; the graft copolymer has a) poly(vinyl acetate) and/or poly (vinyl alcohol) and/or poly(vinyl chloride) and poly(vinyl ester) on b) a polymer chain of polyethylene glycols, polyalkylene glycols, polypropylene glycols, polyisobutylene glycols or polymeth-ylpentene glycols; graft copolymer polyvinyl acetate and/or hydrolysed polyvinyl acetate (polyvinyl alcohol) groups on a polyalkylene oxide (preferably polyethylene oxide); vinylpyrrolidone-vinyl acetate copolymers; vinylpyrrolidone-vinyl acetate copolymer-64+; vinylpyrrolidone-vinyl acetate VA 64; polymethacrylate-based copolymers includes anionic, cationic, and neutral
copolymers based on methacrylic acid and methacrylic/acrylic esters their salts, esters or other derivatives and mixtures thereof;
J. Carriers listed in [0092] and [0093];
K. Methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in A-J, and their esters, salts, and any other chemical derivatives, and mixtures thereof;
L. Any combination of substances specified in items A-K;
wherein co-treating method (solid dispersion preparation method) is selected from:
a) grinding/milling with high energy stress method;
b) grinding/milling with high energy stress and solvent method;
c) media milling method;
d) kneading method;
e) hot-melt method/melting method/fusion method;
f) hot-melt extrusion/hot-stage extrusion method;
g) meltrex method;
h) melt agglomeration method;
i) high-pressure homogenization method;
j) solvent evaporation method;
k) spin-coated films method; electrostatic spinning, electrostatic blowing, electrospraying film casting;
l) spray-drying method;
m) supercritical fluid (SCF) process method;
n) cryogenic techniques method;
o) lyophilization/freeze-drying technique method;
p) spray freezing onto cryogenic fluids method;
q) spray freezing into cryogenic liquids (SFL) method;
r) spray freezing into vapor over liquid (SFV/L) method;
s) ultra-rapid freezing method;
t) precipitation/co-precipitation method;
u) microwave irradiation method;
v) energy input method;
w) heat/shear energy input method;
x) gel entrapment technique;
y) a method comprising: contacting an active pharmaceutical ingredient and a matrix “host substance” to form a solid dispersion under conditions sufficient to form a solid dispersion;
y) combined method.

7. The methods of claims 1-6 when instead of non-covalent complex, solid phase inclusion complex or molecular/solid dispersion or conjugate (of Formula III) are administered and obtained by the methods and techniques described in this invention (i.e., when instead of API being used AAPI or AANPI) and in the patent and scientific literature.

8. The methods of claims 1-7 when the non-covalent complex, molecular/solid dispersion or conjugate is combined with at least one additional active pharmaceutical ingredient (AAPI) active (in vitro, in vivo, ex vivo, in silico) against cancer and selected from: 5-Fluorouracil; Abarelix; Abiraterone; Acetaminophen; Acetaminophen or paracetamol; Acetazolamide; Actiq; Adcretris; Adriamycin RDF; Afinitor; Agomelatine; Agrylin; Albendazole; Aldara; Aldesleukin; Alemtuzumab; Alfuzosin; Alimta; Aliskiren; Alitretinoin; Alkeran; All-trans retinoic acid or tretinoin; Allopurinol; Aloxi; Alpha-Lipoic Acid; Altretamine; Amantadine; Amifostine; Amiloride; Aminoglutethimide; Aminolevulinic Acid; Amiodarone; Amitriptyline; Amlodipine; Amodiaquine; Amsacrine; Anagrelide; Anakinra; Anastrozole; Anexsia; Anzemet; Aprepitant; Aprotinin; Arava; Aredia; Arginine; Arimidex; Aripiprazole; Aromasin; Arranon; Arsenic Trioxide; Artesunate; Arzerra; Asparaginase Erwinia Chrysanthemi; Aspirin; Atenolol; Atorvastatin; Atovaquone; Atrial Natriuretic Peptide; 7Bendamustine; Benserazide; Bepridil; Bevacizumab; Bexarotene; Bexxar; Bezafibrate; BiCNU; Bicalutamide; Biperiden; Blenoxane; Bleomycin; Bortezomib; Bosentan; Bosulif; Bosutinib; Brentuximab Vedotin; Bromfenac; Bromocriptine; Busulfan; Cabazitaxel; Cabergoline; Cabozantinib; Caffeine; Calcitriol; Campath; Camptosar; Candesartan; Capecitabine; Captopril; Carbimazole; Carboplatin; Carfilzomib; Carglumic acid; Carmustine; Carvedilol; Casodex; CeeNU; Celebrex; Celecoxib; Cephalexin; Cerubidine; Cetuximab; Chlorambucil; Chloroquine; Chlorpromazine; Cholecalciferol; Cidofovir; Cilnidipine; Cimetidine; Cinacalcet; Ciprofloxacine; Cisplatin; Citalopram; Cladribine; Clarithromycin; Clofarabine; Clofoctol; Clolar; Clomifene; Clomipramine; Clotrimazole; Colchicine; Cometriq; Conjugated Estrogens; Cosmegen; Crizotinib; Cyclophosphamide; Cytabarine; Cytadren; Cytarabine; Cytosar-U; Cytoxan; DES; DTIC-Dome; Dacarbazine; Dactinomycin; Dalteparin; Danazol; Dapsone; Dasatinib; Daunorubicin; Deferoxamine; Degarelix; Denosumab; Desmopressin; Diclofenac; Diethylstilbestrol; Diflunisal; Digitoxin; Digoxin; Dimethyl Fumarate; Dipyridamole; Disulfiram; Docetaxel; Dolasetron; Donepezil; Doxazosin; Doxorubicin; Doxycycline; Ebastine; Efavirenz; Eflornithine; Elitek; Ellence; Eloxatin; Emcyt; Emend; Enalapril; Enoxaparin; Enzalutamide; Epalrestat; Epirubicin; Erbitux; Eribulin; Erivedge; Erlotinib; Erwinaze; Esomeprazole; Estramustine; Ethacrynic acid; Ethyol; Etodolac; Etoposide; Eulexin; Everolimus; Evista; Exemestane; FOLFIRI; FOLFIRI-Bevacizumab; FOLFIRI-Cetuximab; FOLFIRINOX; FOLFOX; Famotidine; Fareston; Faslodex; Fasudil; Felodipine; Femara; Fenofibrate; Fentanyl; Fingolimob; Fish oil (EPA/DHA); Flubendazole; Fludara; Fludarabine; Fluorouracil; Fluoxetine; Fluspirilene; Flutamide; Fluvastatin; Fluvoxamine; Folinic Acid; Folotyn; Fulvestrant; Fusilev; Gardasil; Gefitinib; Gemcitabine; Gemtuzumab Ozogamicin; Gemzar; Gleevec; Glipizide; Glucarpidase; Glutamine; Goserelin; Granisetron; Griseofulvin; Halaven; Haloperidol; Herceptin; Hexalen; Hycamtin; Hydralazine; Hydrocodone; Hydroxychloroquine; Hymecromone; Ibandronate; Ibritumomab Tiuxetan; Ibuprofen; Iclusig; Idamycin; Idarubicin; Ifex; Ifosfamide; Imatinib; Imipramine; Imiquimod; Imuran; Indomethacin; Ingenol Mebutate; Inlyta; Interferon Alfa-2b; Intron A; Iodixanol; Ipilimumab; Irbesartan; Iressa; Irinotecan; Irinotecan); Istodax; Itraconazole; Ixabepilone; Ixempra; Jakafi; Jevtana; Kadian; Kepivance; Ketoconazole; Ketorolac; Kyprolis; Kytril; Lanreotide; Lansoprazole; Lapatinib; Leflunomide; Letrozole; Leucovorin; Leukeran; Leukine; Leuprolide; Leustatin; Levetiracetam; Levofloxacin; Levulan; Licofelone; Lidocaine; Lithium; Lomustine; Loperamide; Loratadine; Losartan; Lovastatin; Loxoprofen; Lupon Depot; Lysodren; Macitentan; Manidipine; Maraviroc; Masoprocol; Matulane; Mebendazole; Mechlorethamine; Mechloroethamine; Meclofenamate; Mefloquine; Megace; Megestrol; Megestrol acetate; Melatonin; Meloxicam; Melphalan; Memantine; Mepacrine or Quinacrine; Mercaptopurine; Mesna; Metformin; Methazolamide; Methimazole; Methotrexate; Methoxsalen; Methyl Aminolevulinate; Methylnaltrexone; Methylprednisolone; Metoclopramide; Metvixia; Mifepristone; Minocycline; Mirtazapine; Mitomycin; Mitotane; Mitoxantrone; Montelukast; Morphine; Mozobil; Mutamycin; Mycophenolate; Mycophenolic Acid; Myfortic; Myleran; Mylotarg; Nadroparin; Naltrexone; Naproxen; Navelbine; Nelarabine; Nelfinavir; Neulasta; Neumega; Neutroval; Nexavar; Niclosamide; Nifedipine; Nifurtimox; Nilandron; Nilotinib; Nilutamide; Nimodipine; Nipent; Nisodilpine; Nitazoxanide; Nitisinone; Nitroglycerine; Nitroxoline; Nolvadex; Norethandrolone; Noscapine; Novantrone; Nplate; Octreotide; Ofatumumab; Olanzapine; Olsalazine; Omacetaxine; Omeprazole; Oncaspar; Oncovin; Ondansetron; Oprelvekin; Orlistat; Ormeloxifene; Oseltamivir; Ouabain; Oxaliplatin; Oxcarbazepine; Paclitaxel; Palifermin; Palonosetron; Pamidronate; Panitumumab; Pantoprazole; Paraplatin; Pazopanib; Pegaspargase; Pegfilgrastim; Peginterferon Alfa-2b; Pemetrexed; Penfluridol; Pentamidine; Pentostatin; Pentoxifylline; Perjeta; Perphenazine; Pertuzumab; Phentolamine; Phenylbutyrate; Phenytoin; Photofrin; Picato; Pimozide; Pioglitazone; Pipobroman; Pirfenidone; Platinol; Plenaxis; Plerixafor; Ponatinib; Porfimer; Pralatrexate; Pravastatin; Prazosin; Prednisone; Pregabalin; Premarin; Procarbazine; Progesterone; Prograf; Proleukin; Promethazine; Prometrium; Propranolol; Provenge; Purinethol; Pyrimethamine; Pyrvinium pamoate; Quadramet; Quadrivalent Human Papillomavirus Vaccine; Quetiapine; Rabeprazole; Raloxifene; Raltitrexed; Ranolazine; Rapamune; Rasburicase; Regorafenib; Repaglinide; Ribavirin; Rifabutin; Riluzole; Risperidone; Ritonavir; Rituxan; Rituximab; Roflumilast; Romidepsin; Romiplostim; Rosuvastatin; Ruxolitinib; Samarium Sm 153 Lexidronam; Sargramostim; SecreFlo; Secretin; Sensipar; Sertraline; Sildenafil; Simvastatin; Sipuleucel-T; Sirolimus; Sodium Bicarbonate; Sodium Oxybate; Sorafenib; Spironolactone; Sprycel; Stivarga; Streptozocin; Sulfasalazine; Sulindac; Sunitinib; Sutent; Sylatron; Synribo; Tacrolimus; Tadalafil; Tamoxifen; Tarceva; Targretin; Tasigna; Taxol; Taxotere; Telmisartan; Temodar; Temozolomide; Temsirolimus; Teniposide; Terbinafine; Tetrathiomolybdate; Thalidomide; Thalomid; Thiabendazole; Thioguanine; Thioridazine; Ticagrelor; Ticlopidine; Tigecycline; Timolol; Tinzaparin; Tolfenamic acid; Tomudex; Topiramate; Topotecan; Toremifene; Torisel; Tositumomab; Tranexamic acid; Trastuzumab; Trazodone; Treanda; Trelstar Depot; Triamterene; Trifluoperazine; Triptorelin; Trisenox; Tykerb; Ulinastatin; UroXatral; Urokinase; Uvadex; Valproic acid; Valrubicin; Valsartan; Valstar; Vandetanib; VePesid; Vectibix; Velban; Velcade; Vemurafenib; Verapamil; Vercyte; Vinblastine; Vincristine; Vindesine; Vinorelbine; Visipaque; Vismodegib; Voraxaze; Vorinostat; Votrient; Vumon; Warfarin; Xalkori; Xeloda; Xgeva; Xibrom; Xtandi; Yervoy; Zanosar; Zelboraf; Zevalin; Zofran; Zoladex; Zoledronate; Zoledronic Acid; Zolinza; Zometa; Zytiga; tbo-Filgrastim,
and compounds listed in [0111];

9. The methods of claims 1-8 when the non-covalent complex or molecular/solid dispersion or conjugate is combined with at least one additional active pharmaceutical ingredient (AAPI) selected from:
A. inductor, inhibitor or modulator of P-glycoprotein/BCRP/MRP2/MCT1/SNAT2/PEPT1/ABC-transporters which are described in the scientific and patent literature, for example (non-limited examples): Curcumin, Demethoxycurcumin, Bisdemethoxycurcumin, and the like;
B. inductor, inhibitor or modulator of CYP1, CYP2, CYP3 enzymes (non limiting examples: CYP1A1, 1A2, 2A6, 2B6, 2C8, 2C9, 2C19, 2D6, 2E1 or 3A4 enzymes) which are described in the scientific and patent literature, for example (non-limited examples for CYP3A4): sulphaphenazole, quinidine, furafylline, troleandomycin (TAO) and diethyldithiocarbamate (DDC), and the like;
C. dipole modifiers that are able to incorporate into lipid membranes and modify their physicochemical properties, phase segregation character, dipole potential which are described in the scientific and patent literature, for example (non-limited examples): flavanoids, phloretin, floricin, biochanin A, myricetin, quercetin, taxifolin, genistin, genistein, daidzein, catechin, 2,4,6-trihydroxyacetophenone (THAP), floxin B and erythrosine, dihydroquercetin, and the like with or without vitamin C;
D. Hepatoprotective agent which are described in the scientific and patent literature, for example (non-limited examples): according to the different mechanism of action, it can be roughly divided into detoxification drugs (eg. N-acetylcysteine, S-adenosylmethionine and Glutathione), anti-inflammatory drugs (eg. Glycyrrhizic acid preparation, magnesium isoglycyrrhizinate), hepatocyte membrane protector (eg. Polyene phosphatidylcholine), antioxidant drugs (eg. Bicyclol, Silymarin), anticholestatic drug (Ursodeoxycholic acid, Cholestyramine), immunosuppressants (Glucocorticoids) and specific treatment agents (L-carnitine, Anticoagulants), and the like.

10. The methods of claims 1-9 when the non-covalent complex or molecular/solid dispersion or conjugate is combined with at least one additional active pharmaceutical ingredient (AAPI) selected from:
A. autophagy inductor, inhibitor or modulator which are described in the scientific and patent literature, for example (non-limited examples): lithium; carbamazepine; Rapamyrin; Ternsirolimus; Everolimm; Oeforolimus; PP242; Torin 1; Metformin; 15132799; GDC-0980; 501-0941; Perifosine; Fluspirilene; CCE779; Pimozide; Trifluoperazine; Nicardipine; Lithium; L-690330; Carbamazepine; Xmtospongin B; Minoxidil; Clonidine; Verapamil; Loperamide; Amiodarone; Nimodipine; Nitrendipine; Rilmenidine; Bromperidol; Metergoline; Thioridazine; Chlorpromazine; Fludroconisone; Noscapine; Oemastine; Tat-beclin 1 peptide, and the like;
B. autoptosis inductor, inhibitor or modulator which are described in the scientific and patent literature.
C. ferroptosis inductor, inhibitor or modulator which are described in the scientific and patent literature;
D. pyroptosis inductor, inhibitor or modulator which are described in the scientific and patent literature;
E. cell reprogramming agent which are described in the scientific and patent literature;
F. SCOT inductor, inhibitor or modulator which are described in the scientific and patent literature;
G. senolitic agent which are described in the scientific and patent literature;
H. inductor, inhibitor or modulator of ABCA1; ABCB1; AHR; AIFM1; AKR1C₃; AKT1; ALOX12; ALOX5; ALPP; ANXA1; APAF1; AR; ATF2; ATF3; AURKB; BAD; BAK1; BAX; BBC3; BCL2; BCL2L1; BCL2L11; BCL2L2; BECN1; BID; BIRC2; BIRC3; birc5a; BNIP3; CALR; CASP12; CASP2; CASP3; CASP6; CASP7; CASP8; CASP9; CCNB1; CCND1; CCND2; CCND3; CCNE1; CD80; CDC25C; CDC42; CDH1; CDH2; CDK1; CDK2; CDK4; CDK6; CDK7; CDK9; CDKN1A; CDKN1B; CDKN1C; CDKN2B; CFLAR; CHEK-2; CHUK; COX-1; COX-2; CSN2; CTNNBIP1; CYC3B; CycA; CycB; CYCS; CYP19A1; CYP1A1; CYP1A2; CYP1B1; CYP2B6; CYP2C8; CYPIA1; DDIT3; DIABLO; Dox; DUSP10; E2F1; EBV-EA; EDN1; EGF; EGFR; EGR1; ENDOG; ERBB2; ERBB3; ERK1/2; ERP29; ESR1; FADD; Fas; FASLG; FGF2; FN1; FOS; FOXM1; GADD45A; GATA1; GATA2; GCLC; GGT1; GLI1; GPT; GPX1; GRB2; GSK3B; GSTM1; HBG1; HIF1A; HMBS; HNRNPA2B1; HSPA4; HSPA5; HSPB2; ICAM1; IFNG; IGF1; IGF1R; IGF2; IKBKB; IKBKE; IL1B; IL2; IL4; IL6; IL8; ITGA2; ITGAV; ITGB3; JAK1; JAK2; JUN; JUNB; KDR; KLK3; LDHB; LOX; LTA; LTA4H; MAP2; MAP2K1; MAP2K4; MAPK1; MAPK3; MAPK7; MAPK8; MCL1; MDM2; MMP1; MMP14; MMP15; MMP2; MMP3; MMP7; MMP9; MTOR; MYC; MYLK; NDUFS1; NFATC1; NFE2; NFKB1; NFKBIA; NOS2; NOX1; NQO1; NR1H3; ODC1; PARP1; PCNA; PDIA3; PHB; PIK3CA; PIM2; PLAU; PLCG1; PLK1; PMAIP1; POR; PPARG; PRKAB1; PRKACA; PRKCA; PRKCG; PROM1; Proteasome; PSMD9; PTCH1; PTGER3; PTK2; PTPN1; RAC1; RAD17; RAF1; RAP1A; RB1; RELA; RHOA; RHOB; ROCK1; ROS1; RPS6KA3; RPS6KA5; RPS6KB1; SLC2A1; SNAI1; SOD1; SOS1; SP1; SP3; SP4; SPDEF; SRC; STAT3; STAT5B; SULT1A3; Telomerase; TFDP1; TFF1; TGFBR1; TGFBR2; TGFBR3; TIMP1; TIMP2; TNF; TNFRSF10A; TNFRSF10B; TNFRSFlA; TNFRSF1B; TOP1MT; TOP2A; TOP2B; TP53; TP63; TRADD; TUBB3; TUBG1; TWIST1; UCP2; UGT1A; V-ATPases; VCAM1; VEGFA; WEE1; WSB1; XIAP; XIAP.

11. The methods of claims 1-10 when the non-covalent complex or molecular/solid dispersion or conjugate is combined with
a) at least one additional active natutral pharmaceutical ingredient (AANPI) selected from: Terpenoids; Flavonoids; Alkaloids; Polyketides; Polycyclic aromatic natural products; Steroids; Saponin; Oxygen heterocycles; Benzopyranoids; Benzofuranoids; Aliphatic natural products; Aminoacids and peptides; Polypyrroles; Tannins; and compounds listed in [0114]. AANPI may be in the form of a pure substance isolated from a natural source, in the form of an extract, a dried extract, or in the form of a processed natral source, such as a powder and any other form in which it is contained;
b) at least one additional active natutral pharmaceutical ingredient (AANPI) selected from: folate, glycyrrhizic acid, β-sitosterol-β-D-glucoside (Sito-G), asialoorosomucoid, and asialofetuin;
c) at least one of: radiation therapy; photodynamic therapy; photoimmunotherapy; surgery; chemotherapy, cytotoxic chemotherapy; Immunotherapy, immunotherapy with modified gamma delta T cells; hormonal therapy; oncolytic virus therapy; ketogenic diet plus dexamethasone; and the like.

12. The methods of claims 1-11 when active pharmaceutical ingredient (API) and/or additional active pharmaceutical ingredient (AAPI) and/or additional active natutral pharmaceutical ingredient (AANPI) is formulated in form of:
A) a stabilized aqueous formulation which comprises API and/or AAPI and/or AANPI, solvent, surface active agent and cosolvent or a stabilized formulation which comprises API and/or AAPI and/or AANPI solutions in fat/oil or API and/or AAPI and/or AANPI emulsion;
B) The method of claim 10, 11 wherein the compound is a compound of Formula IV in the form of solution/emulsion/suspension comprising:
a) a first pharmaceutical composition comprising a compound of Formula I-III, and;
b) at least one of the following components:
(1) oil phase containing vegetable or/and animal fats;
(2) one or more surfactants;
(3) one or more solvents;
(4) one or more gelling agents;
C) nanoparticles, microparticles, nanocomposites, microcomposites, composites, polymeric nanoparticles (PNPs), liposomes, freeze-dried liposomes, micelles, polymeric micelles, niosomes, solid lipid nanoparticles (SLNs), nanostructured lipid carriers (NLCs), nanoemulsions, microemulsions, emulsions, self-emulsifying (nano or micro) drug delivery systems (SNEDDS, SMEDDS, SEDDS), nanocrystals, co-crystals, mesoporous silica nanoparticles (MSNs), dendrimers, ultradispersions, solid dispersions, solid amorphous dispersions, noncovalent complexes, complexes with polymer, complexes with biopolymer, host-guest complexes, inclusion complexes, solutions, solutions in fat/oil, solutions with cosolvent, solutions with surface active agent/surfactant, solutions with solubilizing agent, polymorphes, lipid-based systems, lipid-based formulations, lipid-based drug delivery systems (LBDDS), gels, colloids, sols, and the like, or in any form described in the scientific and patent literature for example;
D) a food product or beverage, or dietary supplement;
E) wherein API, AAPI, AANPI can be obtained using methods of: A) Reduction Particle size: a) Micronization; b) Nanosuspension; B) Modification of the crystal habit; C) Solid dispersions: a) Eutectic mixtures; b) Solid solutions; c) Amorphous solid solutions; d) Glass solutions and glass suspension; e) Cryogenic techniques; A) Change of pH; B) Use of buffer; C) Derivatization; D) Complexation; E) Salt formation; A) Supercritical fluid process; B) Use of adjuvant like surfactant, solubilizers, cosolvency, hydrotropy, and novel excipients.

13. The methods of claims 1-12 when non-covalent complexes or molecular/solid dispersions or conjugates are used as anticancer pharmaceutical agent, antitumor pharmaceutical agent, antineoplastic pharmaceutical agent, chemotherapeutic agent, or agent that is inductor, inhibitor or modulator of:
1) cell death in cancer cell lines;
2) cell death in senescent cell lines;
3) immunogenic cell death;
4) PAKI-mediated cytostatic autophagy;
5) caspase-dependent apoptosis;
6) WNT-T cell factor (TCF), Hippo and Akt/mTOR pathways;
7) growth and proliferation of cancer cells;
8) cancer stem-like cells.

14. The methods of claims 1-13 when the dosage of the non-covalent complex or molecular/solid dispersion or conjugate
a) exceeds EC 1 (1% of maximal effective concentration for API) for the respective cancer cell line;
b) exceeds EC 5 (5% of maximal effective concentration for API) for the respective cancer cell line;
c) exceeds EC 50 (half maximal effective concentration for API) for the cancer cell line;
d) exceeds EC 90 (90% of maximal effective concentration for API) for the cancer cell line.

15. The method of treatment of the cancer and/or method of manufacture a pharmaceutical dosage form to treat cancer in a human or a mammal or an animal or a bird by administering a therapeutically effective amount of the solid dispersion or the noncovalent complex or conjugate to a human or a mammal or an animal or a bird in need thereof wherein the “carrier (host)” molecule of the solid dispersion, “carrier (host)” molecular part of non-covalent complex or conjugate interacts with the biological membranes of the organism in such a way that the transfer of molecules, ions or metabolites of API, AAPI, AANPI across this biological membrane is improved. Thus, the scope of the invention includes solid dispersions and non-covalent complexes and conjugates with special permeability properties (highly permeable solid dispersions and non-covalent complexes and conjugates).

16. The methods of claims 1-15 when the complex, the molecular/solid dispersion, the conjugate has at least one of the altered properties compared to pure API, AAPI, AANPI:
1) The non-covalent complex, the solid dispersion, the conjugate has a higher bioavailability than pure API, AAPI, AANPI;
2) The non-covalent complex, the solid dispersion, the conjugate has a higher permeability than pure API, AAPI, AANPI;
3) The non-covalent complex, the solid dispersion, the conjugate has a lower toxicity or cytotoxicity than pure API, AAPI, AANPI;
4) The non-covalent complex, the solid dispersion, the conjugate has a different pharmokinetic parameters than API, AAPI, AANPI;
5) The non-covalent complex, the solid dispersion, the conjugate has a improved solubility than API, AAPI, AANPI.

17. The methods of claims 1-16 when The methods of claims 1-16 wherein (at least one of the following):
a) the carrier chemically modified. I.e. carrier is a polymer, biopolymer or synthetic polymer, or methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified, esters and ethers, salts, and any other chemical derivatives thereof;
b) the method of preparation of non-covalent complex, molecular/solid dispersion, or conjugate is selected from listed in the present invention;
c) the complex, the molecular/solid dispersion, the conjugate use for directing a therapeutic agent to a selected cell target in a subject;
d) the therapeutic agent is covalently linked to the carrier;
e) the carrier has a plurality of carboxymethyl functional groups suitable for covalent linkage to the therapeutic agent;
f) the carrier is covalently linked to the therapeutic agent by a phosphoramidate linkage;
g) the carrier is covalently linked to the therapeutic agent by a ethylenediamine linkage;
h) the mass ratio of API, AAPI, AANPI to polymer or carrier is from 1:1 to 1:10000;
i) additional active pharmaceutical ingredient (AAPI) or additional active natutral pharmaceutical ingredient (AANPI) are co-processed with the API to form their joint non-covalent complex or molecular/solid dispersion or conjugate;
j) non-covalent complex or molecular/solid dispersion is obtained by any of the methods described in the description of the invention.

18. The methods of claims 1-17 wherein disease is selected from “Diseases of the blood and bloodforming organs and certain disorders involving the immune mechanism”, “Endocrine, nutritional and metabolic diseases”, “Mental and behavioural disorders”, “Diseases of the nervous system”, “Diseases of the eye and adnexa”, “Diseases of the ear and mastoid process”, “Diseases of the circulatory system”, “Diseases of the respiratory system”, “Diseases of the digestive system”, “Diseases of the skin and subcutaneous tissue”, “Diseases of the musculoskeletal system and connective tissue”, “Diseases of the genitourinary system”, “fungal diseases”, “bacterial diseases”, “viral diseases”, “parasitic diseases”. or any other disease or medical condition of official, traditional or alternative medicine.

19. The methods of claims 1-18 when non-covalent complexes or molecular/solid dispersions or conjugates (alone or in combination with AAPI, AANPI) may be used for a wide range of active agents of the group of ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha 2-adrenergic agonists, analgesics, antipyretics and anti-inflammatory agents, androgens, anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretics, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinsons agents, antiplatelet agents, antiprogestins, antithyroid agents, anti-tussives, antiviral agents, antidepressant, a typical antidepressants, azaspirodecanediones, barbituates, benzodiazepines, benzothiadiazides, beta-adrenergic agonists, beta-adrenergic antagonists, selective beta 1-adrenergic antagonists, selective beta 2-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, haematopoietic agents, histamines, histamine antagonists, 5-hydroxytryptamine antagonists, drugs for the treatment of hyperlipoproteinemia, hypnotics and sedatives, immunosuppressive agents, laxatives, methylxanthines, monoamine oxidase inhibitors, neuromuscular blocking agents, organic nitrates, opiod analgesics and antagonists, pancreatic enzymes, pheno thiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins, hormones, steroids, nucleic acids, antibodies, enzymes, chemoprotective agents and radioprotecting agents;
and (as agent or agent for):
Abortifacient, Acaricidal, Allelopathic, Allergenic, Amnestic, Analgesic, Anesthetic, Anthelmintic, Antiallergic, Antiamebic, Antianemic, Anti-anxiety, Antiarrhythmic, Antiarthritic, Antiasthmatic, Antibacterial, Anticancer, Anticholinergic, Anticholinesterase, Anticonvulsant, Antidementic, Antidepressant, Antidermatitic, Antidiabetic, Antidiarrheic, Antidote, Antiedemic, Antiemetic, Antifertility, Antifungal, Antigout, Antihepatitic, Antihepatotoxic, Anti-HIV, Anti-HSV, Antihyperlipidemic, Antihypertensive, Anti-inflammatory, Antileishmanial, Antileprotic, Antimalarial, Antimicrobial, Antimigraine, Anti-muscle regidity, Antimutagenic, Antimyasthenic, Antineoplastic, Antioxidant, Antiparkinson, Antiprotozoal, Antipsychotic, Antipyretic, Antiseptic, Antispasmodic, Antithrombotic, Anti-tremor, Antitrypanosomal, Antituberculotic, Antitumor, Antitussive, Antiulcerogenic, Antiviral, Anxiogenic, Attractant, Biomarker, Cardiotonic, Carminative, Choleretic, CNS stimulant, Convulsant, Cytotoxic, Defense, Dental, Depilatory, Dermatitic, Diaphoretic, Diuretic, Edematous, Emetic, Emulsifying agent, Enhance flowering, Enhance fruiting, Enhance germination, Enhance leaf growth, Enhance plant growth, Enhance plant growth, Enhance root growth, Enhance stem growth, Essential amino acid, Expectorant, Feeding attractant, Feeding deterrent, Flavor, Genotoxic, Hallucinogenic, Hemolytic, Hemostatic, Hepatotoxic, Herbicidal, Hormonal, Hypnotic, Immunomodulative, Immunostimulant, Immunosuppressant, Induce tremor, Inhibit CYP, Inhibit flowering, Inhibit fruiting, Inhibit germination, Inhibit leaf growth, Inhibit plant growth, Inhibit root growth, Inhibit spore germination, Inhibit stem growth, Insecticidal, Irritant, Laxative, Molluscicidal, Muscle relaxant, Mutagenic, Narcotic, Nematocidal, Neurotoxic, Nonessential amino acid, Nucleic acid, Nutrient, Odor, Other cardiovascular agent, Other digestive organ agent, Other genitourinary agent, Other health agent, Other nervous system agent, Other respiratory tract agent, Oviposition attractant, Oviposition deterrent, Oxytocic, Pediculicidal, Phototoxic, Phytoalexin, Phytotoxic, Pigment, Piscicidal, Pneumotoxic, Pollinator attractant, Psychotomimetic, Repellent, Sedative, Sex attractant, Solvent, Stomachic, Teratogenic, Tonic, Toxic, Tumorigenic, UV shield, Vitamin agent. Supplements Agent, life-extending agent, longevity enhancing agent, Anti-Age Agent, blood thinning agent, Nootropic agent, psychoactive agent, antipsychotic agent;
and (as agent or agent for):
sleep agent; sexual reproduction agent; Reproductive Control Agent; Abortifacient Agent; Abortifacient Agent, Nonsteroidal; Abortifacient Agent, Steroidal; Contraceptive Agent; Contraceptive Agent, Female; Contraceptives, Oral; Contraceptives, Oral, Combined; Contraceptives, Oral, Hormonal; Contraceptives, Oral, Sequential; Contraceptives, Oral, Synthetic; Contraceptives, Postcoital; Contraceptives, Postcoital, Hormonal; Contraceptives, Postcoital, Synthetic; Luteolytic Agent; Menstruation-Inducing Agent; Sperm Immobilizing Agent; Spermatocidal Agent; Contraceptive Agent, Hormonal; Contraceptive Agent, Male; Antispermatogenic Agent; Spermatogenesis-Blocking Agent; Fertility Agent; Fertility Agent, Female; Fertility Agent, Male; Oxytocics; Tocolytic Agent; thinking, speech, reading, writing, behavior agent; observation, hearing, smell, sensory abilities agent; nutrition agent; respiration agent; excretion agent; locomotion, operation of limbs and body parts agent; Musculoskeletal system agent; Circulatory system, Cardiovascular system, Lymphatic system agent; Cardiovascular Agent; Anti-Arrhythmia Agent; Antihypertensive Agent; Calcium Channel Blockers; Cardioplegic Solutions; Cardiotonic Agent; Fibrinolytic Agent; Natriuretic Agent; Nitric Oxide Donors; Potassium Channel Blockers; Sclerosing Solutions; Sodium Channel Blockers; Voltage-Gated Sodium Channel Blockers; Vasoconstrictor Agent; Calcium Channel Agonists; Nasal Decongestants; Vasodilator Agent; Endothelium-Dependent Relaxing Factors; Nervous system agent; Central Nervous System Agent; Abuse-Deterrent Formulations; Aversive Agent; Adjuvants, Anesthesia; Alcohol Deterrents; Analgesics; Analgesics, Non-Narcotic; Analgesics, Short-Acting; Calcitonin Gene-Related Peptide Receptor Antagonists; Dentin Desensitizing Agent; Narcotics; Analgesics, Opioid; Anticonvulsants; Anti-Dyskinesia Agent; Antiparkinson Agent; Aromatic Amino Acid Decarboxylase Inhibitors; Catechol O-Methyltransferase Inhibitors; Antiemetics; Antitussive Agent; Central Nervous System Depressants; Anesthetics; Anesthetics, Combined; Anesthetics, General; Anesthetics, Inhalation; Anesthetics, Intravenous; Anesthetics, Dissociative; Anesthetics, Local; Hypnotics and Sedatives; Sleep Aids, Pharmaceutical; Orexin Receptor Antagonists; Tranquilizing Agent; Anti-Anxiety Agent; Antimanic Agent; Antipsychotic Agent; Central Nervous System Stimulants; Aphrodisiacs; Appetite Stimulants; Appetite Depressants; Convulsants; WakefulnessPromoting Agent; Emetics; Muscle Relaxants, Central; Narcotic Antagonists; Neuroprotective Agent; Nootropic Agent; Psychotropic Agent; Antidepressive Agent; Antidepressive Agent, Second-Generation; Antidepressive Agent, Tricyclic; Hallucinogens; Integumentary system agent; Dermatologic Agent; Antipruritics; Astringents; Emollients; Keratolytic Agent; Photosensitizing Agent; Sunscreening Agent; Haematopoietic and immune systems agent; Blood Agent; Lymphocyte Activation Agent; Lymphocyte Cooperation Agent; Neutrophil Activation Agent; Neutrophil Infiltration Agent; Hematologic Agent; Anticoagulants; Antithrombins; Factor Xa Inhibitors; Antisickling Agent; Blood Substitutes; Plasma Substitutes; Coagulants; Agglutinins; Hemagglutinins; Hemostatics; Anticoagulant Reversal Agent; Antifibrinolytic Agent; Heparin Antagonists; Hematinics; Platelet Aggregation Inhibitors; Immune System Agent; Immunomodulation Agent; Neuroimmunomodulation Agent; Immune Tolerance Agent; Clonal Anergy Agent; Clonal Deletion Agent; Endotoxin Tolerance Agent; Immune Privilege Agent; Self Tolerance Agent; Tachyphylaxis Agent; Transplantation Tolerance Agent; Immunosuppressant; Immunogenetic Agent; Cytokine Agent; Anti-Cytokine Agent; Seroconversion Agent; Immunocompetence Agent; Immune Evasion Agent; Immune Reconstitution Agent; Complement Activation Agent; Complement Pathway, Alternative Agent; Complement Pathway, Classical Agent; Complement Pathway, Mannose-Binding Lectin Agent; Blood Group Incompatibility Agent; Antigen-Antibody Reactions Agent; Antibody-Dependent Enhancement Agent; Anti-Allergic Agent; Mast Cell Stabilizers; Respiratory system agent; Respiratory System Agent; Anti-Asthmatic Agent; Bronchodilator Agent; Bronchoconstrictor Agent; Expectorants; Pulmonary Surfactants; Digestive system agent; Urinary system agent; Reproductive system agent; Endocrine system agent; cell reproduction, division, differentiation, atypia, such as: agent; cancer, malignant tumors such as Carcinoma, Sarcoma, Lymphoma, leukemia, Germ cell tumor, Blastoma (e.g. neuroblastoma, retinoblastoma, nephroblastoma, hepatoblastoma, medulloblastoma, etc.), Melanoma, endothelioma of tissues, organs, parts of organs agent; Antineoplastic Agent; Angiogenesis Inhibitors; Antibiotics, Antineoplastic; Anticarcinogenic Agent; Antimetabolites, Antineoplastic; Antimitotic Agent; Antineoplastic Agent, Alkylating; Antineoplastic Agent, Hormonal; Antineoplastic Agent, Phytogenic; Antineoplastic Agent, Immunological; Immune Checkpoint Inhibitors; MTOR Inhibitors; Myeloablative Agonists; Poly(ADP-ribose) Polymerase Inhibitors; Topoisomerase Inhibitors; Topoisomerase I Inhibitors; Topoisomerase II Inhibitors; cell death, apoptosis, autophagy agent; cellular signaling agent; metabolism of cells and tissues agent; cellular uptake of nutrients agent; sensitivity to the action of hormones, transmitters, neurotransmitters, etc., such as insulin, cortisol, dopamine, and others agent; genetic diseases, chromosomal disorder or genetic syndrome agent; Anti-Inflammatory Agent; Anti-Inflammatory Agent, Non-Steroidal; Cyclooxygenase Inhibitors; Cyclooxygenase 2 Inhibitors; Antifibrotic Agent; Demulcents; Tumor Necrosis Factor Inhibitors; Antiviral Agent; Anti-Retroviral Agent; Anti-HIV Agent; CCR5 Receptor Antagonists; HIV Fusion Inhibitors; HIV Integrase Inhibitors; HIV Protease Inhibitors; Reverse Transcriptase Inhibitors; Viral Fusion Protein Inhibitors; Viral Protease Inhibitors; Coronavirus Protease Inhibitors; HCV NS3-4A Protease Inhibitors; anti-bacterial agent; anti-fungi agent; anti-parasites agent; anti-ectoparasitose agent; Antiprotozoal Agent; Amebicides; Antimalarials; Antitrichomonal Agent; Coccidiostats; Trypanocidal Agent; anti prions agent; anti-toxic agent; anti-allergic agents, including Systemic allergic diseases agent; kinetic exposures and kinetic injuries agent; thermal injuries agent; radiation injuries agent; barotraumas agent; other injuries agent; anti-stress agent; Radiation-Sensitizing Agent; Diagnostic Uses Agent; Prophylaxis Agent; Pre-exposure prophylaxis Agent; Therapeutic Uses Agent; Antiglaucoma Agent; Anti-Infective Agent; Anti-Bacterial Agent; Antitreponemal Agent; Antitubercular Agent; Antibiotics, Antitubercular; beta-Lactamase Inhibitors; Leprostatic Agent; Antifungal Agent; Anti-Infective Agent, Local; Hand Sanitizers; Anti-Infective Agent, Urinary; Antiparasitic Agent; Anthelmintic; Antinematodal Agent; Filaricides; Antiplatyhelmintic Agent; Anticestodal Agent; Schistosomicides; Disinfectants; Contact Lens Solutions; Dental Disinfectants; Root Canal Irrigants; Anti-Obesity Agent; Appetite Depressants; Antirheumatic Agent; Gout Suppressants; Uricosuric Agent; Gastrointestinal Agent; Antacids; Antidiarrheals; Anti-Ulcer Agent; Cathartics; Cholagogues and Choleretics; Guanylyl Cyclase C Agonists; Laxatives; Lipotropic Agent; Genitourinary Agent; Renal Agent; Urological Agent; Lipid Regulating Agent; Hypolipidemic Agent; Anticholesteremic Agent; Hydroxymethylglutaryl-CoA Reductase Inhibitors; PCSK9 Inhibitors; Lipoprotein Lipase Activators; Pharmaceutical Solutions; Dialysis Solutions; Hemodialysis Solutions; Ophthalmic Solutions; Lubricant Eye Drops; Parenteral Nutrition Solutions; Fat Emulsions, Intravenous; Senotherapeutics; Smoking Cessation Agent; Physiological & Psychological Effect Agent; Physiological & Psychological Adaptation Agent; Adaptogen; Nootropic Agent; Psychotropic Drug; Psychoactive Agent; Physiological/Biological Effect Agent; Physiological Adaptation Agent; Acclimatization Agent; Body Temperature Regulation Agent; Sweating Regulation Agent; Thermogenesis Regulation Agent; Shivering Regulation Agent; Torpor Regulation Agent; Estivation Agent; Hibernation Agent; Tissue Survival Agent; Recovery of Function Agent; Regeneration Agent; Bone Regeneration Agent; Brain Regeneration Agent; Liver Regeneration Agent; Nerve Regeneration Agent; Wound Healing Agent; Tissue Regeneration Agent; Organ Regeneration Agent; Aging Agent; Anti-Aging Agent; Skin Aging Agent; Skin Anti-Aging Agent; Immunosenescence Agent; Anti-Immunosenescence Agent; Transplantation Immunology Agent; Graft vs Host Reaction Agent; Host vs Graft Reaction Agent; Histocompatibility Agent; Toxic Agent; Toxicant; Toxin; Cytotoxicity; Antitoxin; Antidote; Antivenin; Cytotoxicity; Pigmentation Agent;
and (as agent or agent for):
ACE inhibitors; Inhibitors Of The Biosynthesis Of Adrenocortical Steroids; Monoamine Oxidase Inhibitors; Inhibitors Of Tubular Transport Of Organic Compounds; Alpha-Adrenergic Antagonists; Beta-Adrenergic Antagonists; Selective Beta 1-Adrenergic Antagonists; Histamine Antagonists; 5-Hydroxytryptamine Antagonists; Opiod Analgesics And Antagonists; Calcium Channel Blockers; Sodium Channel Blockers; Beta Blocking Agent; Calc. Chan. Blocker; Alpha-Adrenergic Agonists; Selective Alpha 2-Adrenergic Agonists; Beta-Adrenergic Agonists; Selective Beta 2-Adrenergic
Agonists; Cholinergic Agonists; Leukocyte Growth Factor Agent; Cytolytic Antibody; Small Molecule Agent; Fluorinated Agent; Sulfur Containing Agent; Chlorinated Agent; Oncological Agent; Myeloid Leukemia Agent; Breast Cancer Agent; Colorectal Cancer Agent; Multiple Myeloma Agent; Testicular Cancer Agent; Hodgkin Lymphoma Agent; Graft-Versus-Host-Disease Agent; Brain Tumors Agent; Melanoma Agent; Lymphoblastic Leukemia Agent; Prostate Cancer Agent; Small Cell Lung Cancer Agent; Ovarian Cancer Agent; Hairy Cell Leukemia Agent; Skin Cancer Agent; Graft-Versus-Host Disease Agent; Chem. Ind. Nausea & Vomiting Agent; Esophageal Cancer Agent; Opiod Analgesic Agent; Hypercalcemia Agent; Non-Small Cell Lung Carcin Agent; Basal Cell Carcinoma Agent; Thrombocytopenia Agent; Non Hodgkin Lymphoma Agent; Bone Cancer Agent; Opioid Analgesic Agent; Bladder Cancer Agent; T-Cell Lymphoma Agent; Lymphocytic Leukemia Agent; Pancreatic Cancer Agent; Parathyroid Carcinoma Agent; Graft-Versus Host Disease Agent; Kidney Cancer Agent; Tumor Lysis Syndrome Agent; Bone Marrow Cancer Agent; Thyroid Cancer Agent; Anal Cancer Agent; Basophilic Leukemia Agent; Biliary Tract Cancer Agent; Blood Cancer Agent; Breast Tumors Agent; Cervical Cancer Agent; Colorectal Tumors Agent; Endometerial Cancer Agent; Eosinophilic Leukemia Agent; Gastric Cancer Agent; Gastrointestinal Tumors Agent; Head Cancer Agent; Lymphoblastic Lymphoma Agent; Mantle Cell Lymphoma Agent; Mesothelioma Agent; Neck Cancer Agent; Neutrophilic Leukemia Agent; Non Small Cell Lung Carcinoma Agent; Pancreatic Tumors Agent; Mineral Supplement; Vitamin; Alimentary Tract Agent; Metabolism Agent; Laxative Agent; Gastrointestinal Agent; Antidiar., Antiinfla., Antiinf. Agent; Stomatological Prep; Antidiabetic Agent; Anti-Obesity Agent; Anabolic Agent; Acid Related Disorders Agent; Antiemetic Agent; Antinauseant Agent; Anti-Infective Agent; Antibacterial Agent; Antimycobacterial Agent; Antiviral Agent; Antimycotic Agent; Anti-Parasitic Agent; Anti-Bacterial Agent; Anti-Fungal Agent; Blood And Blood Forming Organ Agent; Antithrombotic Agent; Antihemorrhagic Agent; Blood Subs. & Perfusion Slns.; Lipid Modifying Agent; Antianemic Agent; Anticoagulant; Antileukemic Agent; Chemotherapeutic Agent; Cardiovascular Agent; Vasoprotective Agent; Cardiac Therapy Agent; Antihypertensive Agent; Peripheral Vasodilator Agent; Diuretic; Renin-Angiotensin Agent; RAS-acting agents; Endocrine System Agent; Endocrine Therapy Agent; Thyroid Therapy Agent; Corticosteroid; Immunosuppressive Agent; Pituit. & Hypoth. Hormones; Calcium Homeostasis Agent; Genito-Urinary And Sex Hormone Agent; Sex Hormones & Modulators Agent; Gynecological Antiinfective Agent; Antiseptic Agent; Urological Agent; Gynecologicals Agent; Musculo-Skeletal Agent; Muscle Relaxant; Topical Products; Other Disorders Of Musculo-Skeletal System Agent; Antiinflammatory Agent; Antirheumatic Agent; Antigout Agent; Treatment Of Bone Diseases Agent; Nervous System Agent; Anesthetic Agent; Analgesic Agent; Psychoanaleptic Agent; Antiepileptic Agent; Misc. Nervous Agent; Anti-Parkinson Agent; Psycholeptic Agent; Antidepressant; Respiratory System Agent; Cough & Cold Prep; Throat Prep; Obstructive Airway Diseases Agent; Antihistamine Agent; Nasal Prep; Other Respir. System Prods; Sensory Organ Agent; Ophthalmological Agent; Otological Agent; Ophthalmol. & Otol. Prep; Alimentary Tract & Metabolism Agent; Anti-Infective Agent; Blood & Blood Forming Organs Agent; Cardiovascular System Agent; Dermatological Agent; Endocrine System Agent; Genito-Urinary & Sex Hormones Agent; Musculo-Skeletal System Agent; Nervous System Agent; Oncological Agent; Respiratory System Agent; Sensory Organs Agent; Nervous System' Agent; Genito-Urinary & Sex Horomone; Blood & Blood Forming Organ Agent; Cardiovascular Agent; Genito-Urinary & Sex Hormone Agent; Sensory Organ Agent; Musculo-Skeletal; Alimentary Tract & Metabolism Agent; Reproductive Agent; Endocrine Agent; AntiInflammatory; Skeletomuscular; Immulogical; Contrast Agent; Alimentary Agent; Antidote; Nsaid; Cardiovascular Diseases Agent; Oncology Agent; Infectious Diseases Agent; Diabetes Agent; Genetic Disorders Agent; Neurology Agent; Respiratory Disorders Agent; Immunology Agent; Dermatology; Rare Disorders Agent; Hematology Agent; Nephrology; Urology; Sleep; Women's Healthcare Agent; Hormonal Disorders Agent; Ophthalmology; Diagnostics; Miscellaneous Agent; Diagnostic Contrast Agent; Veterinary Agent; Adeno-Hypophoseal Hormones; Adrenergic Neuron Blocking Agent; Adrenocortical Steroids; Analgesics; Antipyretics And Anti-Inflammatory Agent; Androgens; Anesthetics; Antiaddictive Agent; Antiandrogens; Antiarrhythmic Agent; Antiasthmatic Agent; Anticholinergic Agent; Anticholinesterase Agent; Anticoagulants; Antidiabetic Agent; Antidiarrheal Agent; Antidiuretics; Antiemetic And Prokinetic Agent; Antiepileptic Agent; Antiestrogens; Antifungal Agent; Antihypertensive Agent; Antimicrobial Agent; Antimigraine Agent; Antimuscarinic Agent; Antineoplastic Agent; Antiparasitic Agent; Antiparkinsons Agent; Antiplatelet Agent; Antiprogestins; Antithyroid Agent; Anti-Tussives; Antiviral Agent; A Typical Antidepressants; Azaspirodecanediones; Barbituates; Benzodiazepines; Benzothiadiazides; Bile Salts; Agent Affecting Volume And Composition Of Body Fluids; Butyrophenones; Agent Affecting Calcification; Catecholamines And Sympathomimetic Agent; Cholinesterase Reactivators; Dermatological Agent; Diphenylbutylpiperidines; Diuretics; Ergot Alkaloids; Estrogens; Ganglionic Blocking Agent; Ganglionic Stimulating Agent; Hydantoins; Agent For Control Of Gastric Acidity And Treatment Of Peptic Ulcers; Haematopoietic Agent; Histamines; Agent For The Treatment Of Hyperlipoproteinemia; Hypnotics And Sedatives; Immunosuppressive Agent; Laxatives; Methylxanthines; Neu-Romuscular Blocking Agent; Organic Nitrates; Pancreatic Enzymes; Pheno Thiazines; Progestins; Prostaglandins; Agent For The Treatment Of Psychiatric Disorders; Retinoids; Agent For Spasticity And Acute Muscle Spasms; Succinimides; Thioxanthines; Thrombolytic Agent; Thyroid Agent; Tricyclic Antidepressants; Agent Affecting Uterine Motility; Vasodilators
and
agent for support health of: a. skin; b. head—eye—ear—nose—mouth—tongue—teeth—lower jaw—face—cheek; c. neck—throat—Adam's apple—shoulders; d. hand—elbow—wrist—hand—fingers—thumb; e. spine—breast—mammary gland—rib cage; f. abdomen—navel—genitals (penis/scrotum or clitoris/vagina)—perineum—anus; g. leg—pelvis—thigh—buttock—knee—shin—calf—foot; h. hair, nails; i. pituitary—duodenum—stomach—gallbladder—intestines—lungs—uterus—bladder—adrenals—parathyroid—liver—pancreas—kidneys—prostate esophagus—spleen—heart—thymus—worm gland—thyroid—testes—ovaries; j. brain; k. anatomical parts included in YTerminologia Anatomica The Second Edition Parts I-V, Chapters 1-16, which is the international standard for human anatomical terminology; 1. other anatomical parts included in MESH terminology;
and
agent for a medical condition like: a. embryo agent; b. fetus agent; c. infantile age agent; d. childhood agent; e. adulthood agent; f. old age agent; g. pregnancy and childbirth agent; h. disability agent; i. coma agent; j. anesthesia agent; k. sleep agent; l. during medical procedures, diagnostics and surgeries; m. other included in MESH terminology
and the like, alone or in combination. Although extensive, this list is not intended to be comprehensive.

20. The methods of claims 1-19 wherein is used for an animal or mammal or bird.
